(19) United States
(12) Patent Application Publication
(10) Pub. No.: US 2024/0174603 A1
(43) Pub. Date: May 30, 2024

(54) MODULATORS OF PROTEIN PHOSPHATASE 2A (PP2A) AND METHODS USING SAME

(71) Applicant: Rappta Therapeutics Oy, Helsinki (FI)

(72) Inventors: George L. Trainor, Wilmington, DE (US); Maria Obdulia Rabal Gracia, Huesca (ES); Laura Fourmois, Montigny Le Bretonneux (FR); Olga Gherbovet, Saint-Hilaire (FR); Frederic Cachoux, Caraman (FR)

(73) Assignee: Rappta Therapeutics Oy, Helsinki (FI)

(21) Appl. No.: 18/273,458

(22) PCT Filed: Feb. 7, 2022
(86) PCT No.: PCT/IB22/00055

§ 371 Date: Jul. 20, 2023

Related U.S. Application Data
(60) Provisional application No. 63/146,785, filed on Feb. 8, 2021.
(60) Provisional application No. 63/191,392, filed on May 21, 2021.

(51) Int. Cl. C07C 311/18; C07C 311/17; C07C 311/19

(52) U.S. Cl. CPC C07C 311/18; C07C 311/17; C07C 311/19; C07B 2200/07

(57) ABSTRACT

The present disclosure relates in part to chemical modulators of protein phosphatase 2A (PP2A), comprising formula (I). The compounds of the present disclosure are useful in treating, preventing, and/or ameliorating cancer, diabetes, autoimmune disease, solid organ transplant rejection, graft vs host disease, chronic obstructive pulmonary disease (COPD), non-alcoholic fatty liver disease, abdominal aortic aneurysm, chronic liver disease, heart failure, neurodegenerative disease, and cardiac hypertrophy.

### CROSS-REFERENCE TO RELATED APPLICATIONS

This application claims priority under 35 U.S.C. § 119(e) to U.S. Provisional Patent Application No. 63/146,785, filed Feb. 8, 2021, and U.S. Provisional Patent Application No. 63/191,392, filed May 21, 2021, all of which are incorporated herein by reference in their entireties.

### BACKGROUND OF THE INVENTION

Protein phosphatase 2A (PP2A) is one of the four major serine threonine phosphatases and is implicated in the negative control of cell growth and division. PP2A holoenzymes are heterotrimeric proteins comprising a structural subunit (A), a catalytic subunit (C), and a regulatory subunit (B). The PP2A heterotrimeric protein phosphatase is a ubiquitous and conserved phosphatase with broad substrate specificity and diverse cellular functions.

PP2A function may be implicated in a variety of pathologies and indications including, but not limited to, cancer, diabetes, autoimmune disease, solid organ transplant rejection, graft vs host disease, chronic obstructive pulmonary disease (COPD), non-alcoholic fatty liver disease, abdominal aortic aneurysm, chronic liver disease, heart failure, neurodegenerative disease, and/or cardiac hypertrophy.

There is thus a need in the art for chemical modulators of PP2A for the treatment, prevention, and/or amelioration of PP2A associated pathologies and/or indications. The present disclosure addresses this need.

### BRIEF SUMMARY OF THE INVENTION

The present disclosure provides certain compounds of formula (I), or a salt, solvate, prodrug, stereoisomer, tautomer, or isotopically labeled derivative thereof, or any mixtures thereof, wherein the substituents in (I) are defined elsewhere herein:

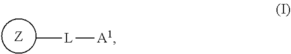

The present disclosure further provides a pharmaceutical composition comprising at least one compound of the present disclosure. In certain embodiments, the pharmaceutical composition further comprises at least one pharmaceutically acceptable carrier.

The present disclosure further provides a method of treating, preventing, and/or ameliorating a PP2A-related disease in a subject, the method comprising administering to a subject in need thereof a therapeutically effective amount of at least one compound of the present disclosure and/or at least one pharmaceutical composition of the present disclosure.

In certain embodiments, the PP2A-related disease is selected from the group consisting of cancer, diabetes, autoimmune disease, solid organ transplant rejection, graft vs host disease, chronic obstructive pulmonary disease (COPD), non-alcoholic fatty liver disease, abdominal aortic aneurysm, chronic liver disease, heart failure, neurodegenerative disease, and cardiac hypertrophy.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “acyl” means a carbonyl attached to an alkenyl, alkyl, aryl, cycloalkyl, heteroaryl, heterocycle, or any other moiety were the atom attached to the carbonyl is carbon.

Examples of acyl groups include formyl, alkanoyl and aroyl. An “acetyl” group refers to a —C(O)CH₃ group. One or more carbons in the acyl residue may be replaced by nitrogen, oxygen or sulfur as long as the point of attachment to the parent molecular moiety remains at the carbonyl. Examples include acetyl, benzoyl, propionyl, isobutyryl, t-butoxycarbonyl, benzyloxycarbonyl and the like.

The term “acylamino” as used herein, alone or in combination, embraces an acyl group attached to the parent moiety through an amino group. An example of an “acylamino” group is acetylamino (CH₃C(O)NH—).

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein, e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In certain embodiments, the compounds and compositions described herein are administered orally.

The terms “alkoxy” or “alkoxyl” as used herein refer to a group of from 1 to 6 carbon atoms of a straight, branched or cyclic configuration and combinations thereof attached to the parent structure through an oxygen. Examples of alkoxy include, but are not limited to, methoxy, ethoxy, propoxy, isopropoxy, cyclopropyloxy, sec-butoxy, isobutoxy, tert-butoxy, cyclohexyloxy and cycloheptyloxy.

In certain embodiments, the term “alkenyl” means an alkyl group having one or more carbon-carbon double bonds. In certain embodiments, the term “C₂₋₆ alkenyl” means an alkenyl moiety having from 2 to 6 carbon atoms.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl, pentenyl, and hexenyl.

The term “alkenylene” as used herein refers to an alkene substituted at two or more positions, such as ethenyl (—CH═CH—). Unless otherwise specified, the term “alkenyl” may include “alkenylene” groups.

In certain embodiments, the term “alkyl” means linear, branched, or cyclic hydrocarbon structures and combinations thereof, and which may be saturated or unsaturated (e.g. partially unsaturated, fully unsaturated). Thus, the term “alkyl” includes the sub-classes alkenyl, alkynyl, cycloalkyl, and the like. Alkyl groups may be optionally substituted as defined herein.

Examples of saturated straight-chain alkyl groups include methyl, ethyl, n-propyl, n-butyl, n-pentyl, n-hexyl, n-heptyl, n-octyl, n-nonyl, and n-decyl and branched-chain alkyl groups include isopropyl, tert-butyl, isobutyl, sec-butyl, and neopentyl. In certain embodiments, alkyl is saturated alkyl having from 2 to 6 carbon atoms. In certain embodiments, a straight chain or branched chain alkyl has 6 or fewer carbon atoms in its backbone (e.g., C₁-C₆ for straight chain, C₃-C₆ for branched chain). The term (C₁-C₆)alkyl may be understood as referring to alkyl groups containing 1 to 6 carbon atoms.

The term “alkylamino” means an alkyl group attached to the parent molecular moiety through an amino group. Alkylamino groups may be mono- or dialkylated, forming groups such as, for example, N-methylamino, N-ethylamino, N,N-dimethylamino, N,N-ethylmethylamino and the like.

The terms “alkylcarbonyl” and “alkoxycarbonyl” mean —C(═O)alkyl or —C(═O)alkoxy, respectively.

The term “alkylthio” means an alkyl thioether (alkyl-S—) group wherein the term alkyl is as defined for alkyl groups and wherein the sulfur may be singly or doubly oxidized.

Examples of alkyl thioether groups include, but are not limited to, methylthio, ethylthio, n-propylthio, isopropylthio, n-butylthio, iso-butylthio, sec-butylthio, tert-butylthio, methanesulfonyl, ethanesulfinyl, and the like.

In certain embodiments, the term “alkynyl” means an alkyl group having one or more carbon-carbon triple bonds. In certain embodiments, the term “C₂₋₆ alkynyl” means an alkynyl moiety having from 2 to 6 carbon atoms.

Examples of unsaturated alkynyl groups include, but are not limited to, ethynyl (ethinyl, —C═CH) and 2-propynyl (propargyl, —CH₂—C═CH).

The term “alkylene” means a straight or branched chain saturated hydrocarbon attached at two or more positions, such as methylene (—CH₂—). Unless otherwise specified, the term “alkyl” may include “alkylene” groups.

Amido (carbamoyl, carbamyl, aminocarbonyl, carboxamide) means a group such as —C(═O)NH₂, —C(═O)NHalkyl, or —C(═O)N(alkyl)₂, wherein alkyl groups are independently amino substituents, as defined for alkyl groups.

The term “amino” means —NH₂.

The terms “aryl” and “heteroaryl” as used herein refer to (i) a phenyl group (or benzene) or a monocyclic 5- or 6-membered heteroaromatic ring containing 1-4 heteroatoms selected from O, N, or S as defined for heterocycles; (ii) a bi cyclic 9- or 10-membered aromatic or heteroaromatic ring system containing 0-4 heteroatoms selected from O, N, or S as defined for carbocycles or heterocycles; or (iii) a tricyclic 13- or 14-membered aromatic or heteroaromatic ring system containing 0-5 heteroatoms selected from O, N, or S as defined for carbocycles or heterocycles. The aromatic 6- to 14-membered carbocyclic rings include, but are not limited to, benzene, naphthalene, anthracene, indane, tetralin, and fluorene and the 5- to 10-membered aromatic heterocyclic rings include, but are not limited to, imidazole, pyridine, indole, thiophene, benzopyranone, thiazole, furan, benzimidazole, quinoline, isoquinoline, quinoxaline, pyrimidine, pyrazine, tetrazole and pyrazole. As used herein aryl and heteroaryl refer to residues in which one or more rings are aromatic, but not all need be.

The term “arylalkyl” as used herein refers to a substituent in which an aryl residue is attached to the parent structure through alkyl. Examples of arylalkyl include, but are not limited to, benzyl, phenethyl and the like. Heteroarylalkyl refers to a substituent in which a heteroaryl residue is attached to the parent structure through alkyl. In certain embodiments, the alkyl group of an arylalkyl or a heteroarylalkyl is an alkyl group of from 1 to 6 carbons.

Examples include, e.g., pyridinylmethyl, pyrimidinylethyl and the like.

The term “azido” as used herein means —N₃.

The term “carbamate” as used herein refers to an ester of carbamic acid (—NHCOO—) which may be attached to the parent molecular moiety from either the nitrogen or acid end, and which may be optionally substituted as defined herein.

The term “carbonyl” as used herein refers to a —C(O)— group and includes formyl (—C(O)H).

The terms “carboxyl” or “carboxy” as used herein refer to —C(O)OH or the corresponding “carboxylate” anion, such as is in a carboxylic acid salt.

The term “co-administered” refers to simultaneous administration in the same formulation or in two different formulations via the same or different routes or sequential administration by the same or different routes. By “sequential” administration is meant a time difference of from seconds, minutes, hours or days between the administration of the two or more separate compounds.

The term “combination therapy” means the administration of two or more therapeutic agents to treat a therapeutic condition or disorder described in the present disclosure. Such administration encompasses co-administration of these therapeutic agents in a substantially simultaneous manner, such as in a single formulation (e.g., a capsule or injection) having a fixed ratio of active ingredients or in multiple, separate dosage forms for each active ingredient. In addition, such administration also encompasses use of each type of therapeutic agent in a sequential manner. In either case, the treatment regimen will provide beneficial effects of the drug combination in treating the conditions or disorders described herein.

The term “comprising” is used in this specification to mean including the feature(s) or act(s) followed thereafter, without excluding the presence of one or more additional features or acts.

The term “cycloalkyl” or alternatively, “carbocycle”, alone or in combination, means a saturated or partially saturated monocyclic, bicyclic or tricyclic alkyl group wherein each cyclic moiety may contain from 3 to 12 carbon atom ring members and which may optionally be a benzo fused ring system which is optionally substituted as defined herein. In certain embodiments, cycloalkyl comprise from 3 to 7 carbon atoms or from 3 to 6 carbon atoms.

Examples of saturated monocyclic cycloalkyl groups include, but are not limited to, cyclopropyl, cyclobutyl, cyclopentyl, cyclohexyl, cycloheptyl, methylcyclopropyl, dimethylcyclopropyl, methylcyclobutyl, dimethylcyclobutyl), methylcyclopentyl, dimethylcyclopentyl and methylcyclohexyl.

Examples of saturated monocyclic cycloalkyl groups include, but are not limited to, cyclopropenyl, cyclobutenyl, cyclopentenyl, cyclohexenyl, methylcyclopropenyl, dimethylcyclopropenyl, methylcyclobutenyl, dimethylcyclobutenyl, methylcyclopentenyl, dimethylcyclopentenyl and methylcyclohexenyl.

Examples of bicyclic cycloalkyl groups include, but are not limited to, tetrahydronapthyl, indanyl, octahydronaphthyl, 2,3-dihydro-1H-indenyl, decalinyl and the like. “Bicyclic” and “tricyclic” as used together with “cycloalkyl” are intended to include both fused ring systems, such as decahydronaphthalene, octahydronaphthalene as well as the multicyclic (multicentered) saturated or partially unsaturated type, including spiro-ring fused systems. Examples of bicyclic and tricyclic types of isomer are bicyclo[1,1,1]pentane, norbornane, camphor, adamantane, bicyclo[3,2,1]octane, and [4,4.1]-bicyclononane.

The terms “cyano,” “nitrile,” or “carbonitrile” as used herein means —CN.

The term “diastereomers” as used herein refers to stereoisomers that have at least two asymmetric atoms, but which are not mirror-images of each other.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The terms “effective amount” or “therapeutically effective amount” as used herein, refer to a sufficient amount of at least one compound being administered which achieve a desired result, e.g., to relieve to some extent one or more symptoms of a disease or condition being treated. In certain instances, the result is a reduction and/or alleviation of the signs, symptoms, or causes of a disease, or any other desired alteration of a biological system. In specific instances, the result is a decrease in the growth of, the killing of, or the inducing of apoptosis in at least one abnormally proliferating cell, e.g., a cancer cell. In certain instances, an “effective amount” for therapeutic uses is the amount of the composition comprising a compound as set forth herein required to provide a clinically significant decrease in a disease. An appropriate “effective” amount in any individual case is determined using any suitable technique, such as a dose escalation study.

The terms “ester,” “carboxylate,” “carboxylic acid ester,” or “oxycarbonyl” as used herein means —C(═O)Oalkyl, wherein alkyl is an ester substituent defined for alkyl above. Examples of ester groups include, but are not limited to, —C(═O)OCH₃, —C(═O)OCH₂CH₃, —C(═O)OC(CH₃)₃, and —C(═O)OPh.

The terms “halo” or “halogen” as used herein means, alone or in combination, fluorine, chlorine, bromine, or iodine. In certain embodiments, halo may be fluorine or chlorine.

The term “haloalkoxy” means a haloalkyl group attached to the parent molecular moiety through an oxygen atom.

The term “haloalkyl” as used herein refers to an alkyl radical having the meaning as defined above wherein one or more hydrogens are replaced with a halogen. In certain embodiments haloalkyl is monohaloalkyl, dihaloalkyl and polyhaloalkyl group. Examples of haloalkyl radicals include, but are not limited to, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” means a haloalkyl group attached at two or more positions. Examples include, but are not limited to, fluoromethylene (—CFH—), difluoromethylene (—CF₂—) and chloromethylene (—CHCl—).

The term “heterocycle” and, interchangeably, “heterocycloalkyl” means a cycloaliphatic or aryl carbocycle residue in which from one to four carbons is replaced by a heteroatom selected from the group consisting of N, O and S. The nitrogen and sulfur heteroatoms may optionally be oxidized, and the nitrogen heteroatom may optionally be quaternized. In certain embodiments, the heterocycle is non-aromatic. In further embodiments, the heterocycle is aromatic.

Examples of heterocycles include, but are not limited to, aziridine, azetidine pyrrolidine, pyrazole, pyrrole, indole, quinoline, isoquinoline, tetrahydroisoquinoline, benzofuran, benzodioxan, benzodioxole, tetrazole, morpholine, thiazole, pyridine, pyridazine, pyrimidine, thiophene, furan, oxazole, oxazoline, isoxazole, dioxane, tetrahydrofuran and the like. Examples of heterocyclyl residues include, but are not limited to, piperazinyl, piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl (also historically called thiophenyl), benzothienyl, thiamorpholinyl, oxadiazolyl, triazolyl and tetrahydroquinolinyl. It is to be noted that heteroaryl is a subset of heterocycle in which the heterocycle is aromatic. An oxygen heterocycle is a heterocycle containing at least one oxygen in the ring; it may contain additional oxygens, as well as other heteroatoms. A sulphur heterocycle is a heterocycle containing at least one sulphur in the ring; it may contain additional sulphurs, as other heteroatoms. Oxygen heteroaryl is a subset of oxygen as other heteroatoms. Oxygen heteroaryl is a subset of oxygen heterocycle; examples include furan and oxazole. Sulphur heteroaryl is a subset of sulphur heterocycle; examples include, but are not limited to, thiophene and thiazine. A nitrogen heterocycle is a heterocycle containing at least one nitrogen in the ring; it may contain additional nitrogens, as well as other heteroatoms. Examples include, but are not limited to, piperidine, piperazine, morpholine, pyrrolidine and thiomorpholine. Nitrogen heteroaryl is a subset of nitrogen heterocycle; examples include, but are not limited to, pyridine, pyrrole and thiazole. The heterocycle groups may be optionally substituted unless specifically prohibited.

The term “hydroxy” means —OH.

As used herein, the term “increase” or the related terms “increased,” “enhance” or “enhanced” may refer to a statistically significant increase, and the terms “decreased,” “suppressed,” or “inhibited” to a statistically significant decrease. For the avoidance of doubt, an increase generally refers to at least a 10% increase in a given parameter, and can encompass at least a 20% increase, 30% increase, 40% increase, 50% increase, 60% increase, 70% increase, 80% increase, 90% increase, 95% increase, 97% increase, 99% or even a 100% increase over the control, baseline, or prior-in-time value. Inhibition generally refers to at least a 10% decrease in a given parameter, and can encompass at least a 20% decrease, 30% decrease, 40% decrease, 50% decrease, 60% decrease, 70% decrease, 80% decrease, 90% decrease, 95% decrease, 97% decrease, 99% or even a 100% decrease over the control value.

The term “imino” as used herein means ═N—.

As used herein, the term “modulate” means to increase or decrease the activity of PP2A. In certain embodiments, compounds according to one or more embodiments disclosed in this specification may increase the activity of specific PP2A holoenzymes while decreasing the activity of other PP2A heterotrimers.

The term “nitro” as used herein means —NO₂.

The term “optionally substituted” may be used interchangeably with “unsubstituted or substituted”. The term “substituted” means the replacement of one or more hydrogen atoms in a specified group with a specified radical. In certain embodiments, 1, 2 or 3 hydrogen atoms are replaced with a specified radical. In the case of alkyl and cycloalkyl, more than three hydrogen atoms can be replaced by fluorine. In certain embodiments, all available hydrogen atoms may be replaced by fluorine. Two substituents may be joined together to form a form a three to seven membered non-aromatic carbocycle or heterocycle consisting of zero to three heteroatoms, for example forming methylenedioxy or ethylenedioxy. In certain embodiments, the formed carbocyclic or heterocyclic ring is fused ring or spiro ring.

The above groups, whether alone or part of another substituent, may themselves optionally be substituted with one or more groups selected from themselves and the additional substituents listed below. Further, the substituents listed below may themselves be substituents.

The term “oxo,” when referred to as a substituent itself, refers to double bonded oxygen (═O).

The terms “oxy” or “oxa” as used herein means —O—.

As used herein, the term “patient” means all mammals including humans. Examples of patients include humans, cows, dogs, cats, goats, sheep, pigs, and rabbits. In some embodiments, the patient is a human.

The term “pharmaceutically acceptable salt” may refer to salts prepared from pharmaceutically acceptable non-toxic acids or bases including inorganic acids and bases and organic acids and bases. When the compounds disclosed in this specification are basic, salts may be prepared from pharmaceutically acceptable non-toxic acids including inorganic and organic acids. Suitable pharmaceutically acceptable acid addition salts for the compounds disclosed in this specification include acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. When the compounds contain an acidic side chain, suitable pharmaceutically acceptable base addition salts for the compounds of the present invention include, but are not limited to, metallic salts made from aluminum, calcium, lithium, magnesium, potassium, sodium and zinc or organic salts made from lysine, arginine, N,N′ dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

The terms “prevent,” “preventing” or “prevention,” and other grammatical equivalents as used herein, include preventing additional symptoms, preventing the underlying metabolic causes of symptoms, inhibiting the disease or condition, e.g., arresting the development of the disease or condition and are intended to include prophylaxis. The terms further include achieving a prophylactic benefit. For prophylactic benefit, the compositions are optionally administered to an individual at risk of developing a particular disease, to an individual reporting one or more of the physiological symptoms of a disease, or to an individual at risk of reoccurrence of the disease.

The term “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are non-superimposable mirror images of one another.

The terms “sulfonate,” “sulfonic acid,” and “sulfonic,” as used herein mean the —SO₃H group and its anion as the sulfonic acid is used in salt formation.

The term “sulfanyl” as used herein means —S—.

The term “sulfinyl” as used herein means —S(O)—.

The term “sulfonyl” as used herein means —S(O)₂—.

The term “sulfonamido” (sulfinamoyl; sulfonic acid amide; sulfonamide) means —S(═O)₂NH₂, —S(═O)₂NH(alkyl), —S(═O)₂N(alkyl)₂, wherein alkyl are independently amino substituents, as defined for alkylamino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

The term “sulfonimidamide” may be understood as referring to a group —NHS((═O)(NR¹⁸))—, wherein in certain Ion-limiting embodiments R¹⁸ is as defined elsewhere herein.

The term “tautomer” or “tautomeric form” refers to structural isomers of different energies which are interconvertible via a low energy barrier. Some non-limiting examples of proton tautomers (also known as prototropic tautomers) include interconversions via migration of a proton, such as keto-enol and imine-enamine isomerizations. Valence tautomers include interconversions by reorganization of some of the bonding electrons.

Unless otherwise stated, all tautomeric forms of the compounds disclosed herein are within the scope of the invention.

The terms “thia” and “thio” as used herein refers to a —S— group or an ether wherein the oxygen is replaced with sulfur. The oxidized derivatives of the thio group, namely sulfinyl and sulfonyl, are included in the definition of thia and thio.

The terms “treat,” “treating” or “treatment,” and other grammatical equivalents as used herein, include alleviating, inhibiting or reducing symptoms, reducing or inhibiting severity of, delaying onset of, delaying recurrence of, abating or ameliorating a disease or condition symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms further include achieving a therapeutic benefit. By therapeutic benefit is meant eradication or amelioration of the underlying disorder being treated, and/or the eradication or amelioration of one or more of the physiological symptoms associated with the underlying disorder such that an improvement is observed in the individual.

Unless otherwise stated or depicted, structures of compounds according to one or more embodiments disclosed in this specification are also meant to include all stereoisomeric (e.g., enantiomeric, diastereomeric, and cis-trans isomeric) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and cis-trans isomeric (or conformational) mixtures of the present compounds are within the scope of the present disclosure. The configuration of any carbon-carbon double bond appearing herein is selected for convenience only and is not intended to designate a particular configuration; thus a carbon-carbon double bond depicted arbitrarily herein as trans may be cis, trans, or a mixture of the two in any proportion. Unless otherwise stated, all tautomeric forms of the compounds according to one or more embodiments disclosed in this specification are within the scope of the present disclosure.

Additionally, the compounds of Formula (I) can exist in unsolvated as well as solvated forms with pharmaceutically acceptable solvents such as water, ethanol, and the like. In general, the solvated forms are considered equivalent to the unsolvated forms.

### Compounds

In one aspect, the present disclosure relates to a compound of formula (I), or a salt, solvate, enantiomer, diastereoisomer, isotopologue, or tautomer thereof:

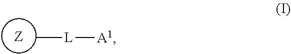

wherein:
A¹ is selected from the group consisting of

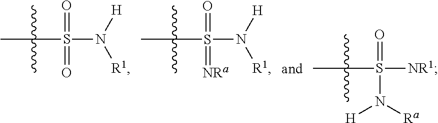

L is a linker comprising a bond, C₁-C₆ alkylene, or C₂-C₆ alkenylene;
ring Z is optionally substituted phenyl, optionally substituted naphthyl, optionally substituted pyridyl, or optionally substituted cyclohexyl,
   wherein each optional substituent is at least one substituent selected from the group consisting of C₁-C₆ alkyl, C₁-C₆ haloalkyl, C₁-C₆ aminoalkyl, C₁-C₆ hydroxyalkyl, C₂-C₆ alkenyl, C₂-C₆ alkynyl, C₁-C₆ alkoxy, C₁-C₆ haloalkoxy, C₆-C₁₀ aryl, C₄-C₁₀ heteroaryl, C₆-C₁₀ aryloxy, C₄-C₁₀ heteroaryloxy F, Cl, Br, I, OH, CN, N(R^{a})(R^{b}), NO₂, C(═O)OR^{a}, and C(═O)N(R^{a})(R^{b}),
   wherein each optional C₆-C₁₀ aryl, C₄-C₁₀ heteroaryl, C₄-C₁₀ heteroaryloxy, or C₆-C₁₀ aryloxy substituent is independently optionally substituted with at least one substituent selected from the group consisting of C₁-C₃ alkyl, C₁-C₃ haloalkyl, C₁-C₃ alkoxy, C₁-C₃ haloalkoxy, F, Cl, Br, I, OH, CN, N(R^{a})(R^{b}), NO₂, OH, C(═O)OR^{a}, and C(═O)N(R^{a})(R^{b}),
   wherein two adjacent substituents of ring Z may optionally combine to provide a 5 to 8-membered fused ring;
R¹ is

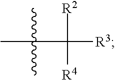

R² is selected from the group consisting of optionally substituted C₁-C₆ alkyl, optionally substituted C₁-C₆ haloalkyl, optionally substituted C₁-C₆ aminoalkyl, optionally substituted C₁-C₆ hydroxyalkyl, optionally substituted C₂-C₆ alkenyl, optionally substituted C₅-C₈ cycloalkenyl, optionally substituted C₂-C₆ alkynyl, optionally substituted C₁-C₆ alkoxy, optionally substituted C₁-C₆ haloalkoxy, optionally substituted C₃-C₁₂ cycloalkyl, optionally substituted C₂-C₁₂ heterocycloalkyl, optionally substituted C₆-C₁₀ aryl, and optionally substituted C₄-C₁₀ heteroaryl,
   wherein each optional substituent in R² is independently selected from the group consisting of C₁-C₃ alkyl, C₁-C₃ haloalkyl, C₁-C₃ alkoxy, C₃-C₅ cycloalkoxy, C₃-C₁₂ heterocycloalkyl, C₁-C₃ haloalkoxy, F, Cl, Br, OR^{b}, CN, NO₂, C(═O)OR^{a}, C(═O)N(R^{a})₂, S(═O)₂R^{b}, NR^{a}S(═O)₂R^{b}, C₄-C₁₀ heteroaryl, benzyl, and phenyl, wherein the benzyl and/or phenyl are each independently optionally substituted with at least one substituent selected from the group consisting of C₁-C₆ alkyl, C₁-C₆ alkoxy, C₁-C₃ haloalkyl, C₁-C₃ haloalkoxy, and halogen;
R³ is selected from the group consisting of H, —C(═O)R^{a}, —C(═O)OR^{a}, —C(═O)N(R^{a})₂, NR^{a}C(═O)OR^{b}, optionally substituted C₁-C₆ alkyl, optionally substituted C₁-C₆ alkoxy, optionally substituted C₁-C₆ haloalkyl, optionally substituted C₁-C₆ aminoalkyl, optionally substituted C₁-C₆ hydroxyalkyl, optionally substituted C₃-C₁₂ cycloalkyl, and optionally substituted C₂-C₁₂ heterocycloalkyl,
   wherein each optional substituent in R³ is selected from the group consisting of C₁-C₃ alkyl, C₂-C₁₂ heterocycloalkyl, C₁-C₃ haloalkyl, C₁-C₃ alkoxy, C₁-C₃ haloalkoxy, benzyl, C₆-C₁₀ aryl, C₄-C₁₀ heteroaryl, F, Cl, Br, OH, CN, NO₂, C(═O)OR^{a}, C(═O)N(R^{a})(R^{b}), NR^{a}C(═O)OR^{a}, S(═O)₂R^{b}, and NR^{a}S(═O)₂R^{b},
R⁴ is H or C₁-C₆ alkyl;
each occurrence of R^{a} is independently selected from the group consisting of H, C₁-C₆ alkyl, C₃-C₆ cycloalkyl, benzyl, and C₆-C₁₀ aryl;
each occurrence of R^{b} is independently selected from the group consisting of H, optionally substituted C₁-C₆ alkyl, optionally substituted C₂-C₁₂ heterocycloalkyl, optionally substituted C₂-C₅ heteroaryl, optionally substituted benzyl, optionally substituted phenyl, and optionally substituted naphthyl,
   wherein each optional substituent in R^{b} is independently selected from the group consisting of C₁-C₃ alkyl, C₁-C₃ haloalkyl, C₁-C₃ alkoxy, C₁-C₃ haloalkoxy, F, Cl, Br, I, OH, CN, NO₂, C(═O)OR^{a}, and C(═O)N(R^{a})₂;
wherein no more than one of R² and R³ is CH₂OH;
wherein if R³ is H and R⁴ is H, R³ is CH₃ and R⁴ is H, or R³ is H and R⁴ is CH₃, then one of the following applies:
   (a) R² comprises at least one substituent selected from the group consisting of C₆-C₁₀ aryl and C₄-C₁₀ heteroaryl, wherein the aryl and/or heteroaryl are each independently substituted with at least one substituent selected from the group consisting of OR, N(R^{a})(R^{a}), C₁-C₆ alkyl, C₁-C₃ haloalkyl, C₁-C₃ haloalkoxy, C₂-C₁₂ heterocycloalkyl, F, Cl, Br, and S(═O)₂R^{b}; or
   (b) R² comprises at least one substituent selected from the group consisting of C₃-C₆ cycloalkyl and C₂-C₁₂ heterocycloalkyl, and at least one substituent selected from the group consisting of C₆-C₁₀ aryl and C₄-C₁₀ heteroaryl;
wherein if L is a bond, R³ is H, and R⁴ is H, and A¹ is

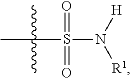

then the optionally substituted C₂-C₁₂ heterocycloalkyl in R² comprises no more than one phenyl which is substituted with at least one halogen;
wherein if Z is optionally substituted phenyl, L is a bond, A¹ is

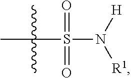

and R² is optionally substituted C₆-C₁₀ aryl, then one of the following applies:
(a) the phenyl in Z is substituted with at least one substituent selected from the group consisting of C₁-C₆ haloalkyl, C₁-C₆ aminoalkyl, C₁-C₆ hydroxyalkyl, C₂-C₆ alkenyl, C₂-C₆ alkynyl, C₁-C₆ alkoxy, C₁-C₆ haloalkoxy, C₆-C₁₀ aryl, C₃-C₆ heterocycloalkyl, C₄-C₁₀ heteroaryl, C₆-C₁₀ aryloxy, C₄-C₁₀ heteroaryloxy F, Cl, Br, I, OH, CN, N(R^{a})(R^{b}), NO₂, C(═O)OR^{a}, and C(═O)N(R^{a})(R^{b}), and the C₆-C₁₀ aryl in R² is substituted with at least one substituent selected from the group consisting of C₁-C₃ haloalkyl, C₁-C₃ alkoxy, C₃-C₅ cycloalkoxy, C₃-C₁₂ heterocycloalkyl, C₁-C₃ haloalkoxy, F, Cl, Br, OR, CN, NO₂, C(═O)OR^{a}, C(═O)N(R^{a})₂, S(═O)₂R^{b}, NR^{a}S(═O)₂R^{b}, C₄-C₁₀ heteroaryl, and benzyl;
(b) the phenyl in Z is substituted with at least one substituent, and the C₆-C₁₀ aryl in R² is substituted with at least two substituents; or
(c) the phenyl in Z is substituted with at least one trifluoromethoxy substituent, the C₆-C₁₀ aryl in R² is substituted with at least one substituent, and R³ is optionally substituted C₁-C₆ aminoalkyl;
wherein if Z is 4-trifluoromethoxyphenyl, L is a bond, and A¹ is

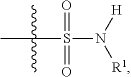

then each of the following apply:
(a) if R³ and R⁴ are each independently H, then R² is not selected from the group consisting of phenyl, 2-difluoromethoxyphenyl, 2-methylphenyl, 2-cyanophenyl, 2-methoxyphenyl, 2-trifluoromethoxyphenyl, 2-chlorophenyl, 2-fluorophenyl, 2-trifluoromethylphenyl, 3-methylphenyl, 3-methoxyphenyl, 3-bromophenyl, 3-chlorophenyl, 3-trifluoromethoxyphenyl, 3-trifluoromethylphenyl, 4-isobutyramido-phenyl, 4-tert-butylphenyl, 4-methoxyphenyl, 4-chlorophenyl, 4-bromophenyl, 4-carboxyphenyl, 4-trifluoromethylphenyl, 4-methylphenyl, 4-fluorophenyl, 2-fluoro-3-trifluoromethylphenyl, 2,3-dimethylphenyl, 2,3-dichlorophenyl, 2-methyl-4-chlorophenyl, 2-chloro-4-fluorophenyl, 2,4-dimethoxyphenyl, 2-methoxy-4-(2-methoxy-5-fluorophenyl)phenyl, 2-methoxy-4-chlorophenyl, 2-fluoro-4-trifluoromethylphenyl, 2,6-difluorophenyl, 2-chloro-6-methylphenyl, 2,6-dimethoxyphenyl, 3,4-ethylenedioxyphenyl, 3-fluoro-4-trifluoromethylphenyl, 3,4-difluorophenyl, 3-fluoro-4-methylphenyl, 3-methoxy-4-ethoxyphenyl, 3,4-dimethoxyphenyl, 3-chloro-4-methylphenyl, 3-methyl-4-fluorophenyl, 3-chloro-4-fluorophenyl, 3,5-difluorophenyl, 3,5-dichlorophenyl, 3,5-dimethoxyphenyl, 3,5-dimethylphenyl, 3,6-dichlorophenyl, 3,6-difluorophenyl, 3,6-dimethoxyphenyl, 3-chloro-6-methylphenyl, 2,3,4-trifluorophenyl, 3,4,5-trifluorophenyl, 2,6-difluoro-3-chlorophenyl, 1-((5-ethanolyl)thiophen-2-yl)cyclopentyl, and 1-(thiophen-2-yl)cyclopentyl;
(b) if R³ is H and R⁴ is methyl or R³ is methyl and R⁴ is H, then R² is not selected from the group consisting of phenyl, naphthyl, 3-methoxyphenyl, 2,4-difluorophenyl, 3,4-methylenedioxyphenyl, 3,4-(1,2-ethylenedioxy)phenyl, 3-fluoro-4-methoxyphenyl, 3,6-dimethoxyphenyl, 2-methylphenyl, 2-methoxyphenyl, 4-methylphenyl, 4-bromophenyl, 4-fluorophenyl, 4-methoxyphenyl, 4-ethoxyphenyl, 4-(methanesulfonyl)phenyl, 4-(3-imidazolyl)phenyl, and 4-(2-(1,2,4-triazol)-yl)phenyl;
(c) if R³ is H and R⁴ is ethyl or R³ is selected from the group consisting of ethyl and cyclopropyl and R⁴ is H, then R² is not selected from the group consisting of phenyl and 4-methoxyphenyl;
(d) if R³ is selected from the group consisting of hydroxymethyl, cyano, 1-(1,2,4-triazol)-yl, and 1-(1,2,5-triazol)-yl, and R⁴ is H, then R² is not phenyl;
(e) if R³ is CH₂C(═O)OH and R⁴ is H, then R² is not selected from the group consisting of phenyl, 2-chlorophenyl, 4-methylphenyl, 4-fluorophenyl, 4-chlorophenyl, 4-tert-butylphenyl, 4-methoxyphenyl, and 3,4-dimethoxyphenyl;
(f) if R³ is CH₂C(═O)OCH₃ and R⁴ is H, then R² is not selected from the group consisting of cyclohexyl, phenyl, 4-chlorophenyl, 4-tert-butylphenyl, 4-methylphenyl, 4-bromophenyl, 4-fluorophenyl, and 3-bromophenyl;
(g) if R³ is CH₂C(═O)OCH₂CH₃ and R⁴ is H, then R² is not selected from the group consisting of phenyl, 2-chlorophenyl, 4-fluorophenyl, and 4-bromophenyl;
(h) if R³ is CH₂(C═O)OCH₂Ph and R⁴ is H, then R² is not 2-chlorophenyl;
(i) if R³ is CH₂(C═O)OCH(CH₃)₂ and R⁴ is H, then R² is not 2-nitrophenyl; and
(j) if R³ is selected from the group consisting of C(═O)NH₂, C(═O)OCH₃, and C(═O)OCH₂CH₃ and R⁴ is H, then R² is not phenyl; and
wherein if Z is 4-difluoromethoxyphenyl, L is a bond, and A¹ is

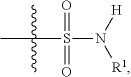

then each of the following apply:
(a) if R³ is CH₂(C═O)OH and R⁴ is H, then R² is not 2-chlorophenyl;
(b) if R³ is CH₂(C═O)OCH₃ and R⁴ is H, then R² is not selected from the group consisting of cyclohexyl, 4-chlorophenyl, and 4-bromophenyl;
(c) if R³ is CH₂(C═O)OCH₂CH₃ and R⁴ is H, then R² is not selected from the group consisting of phenyl, 2-chlorophenyl, and 4-bromophenyl; and
(d) if R³ is CH₂(C═O)OCH₂Ph and R⁴ is H, then R² is not 2-chlorophenyl.

In certain embodiments, L is —CH₂—. In certain embodiments, L is —CH₂CH₂—. In certain embodiments, L is —CH═CH—.

In certain embodiments, A¹ is

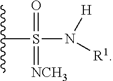

In certain embodiments, A₁ is

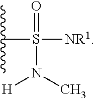

In certain embodiments, R¹ is selected from the group consisting of

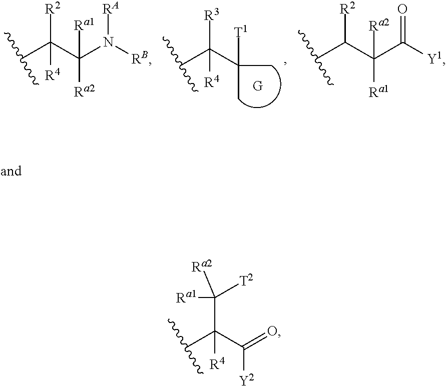

wherein:
R², if present, is selected from the group consisting of optionally substituted C₆-C₁₀ aryl and optionally substituted C₄-C₁₀ heteroaryl,
   wherein each optional substituent in R² is independently selected from the group consisting of C₁-C₃ alkyl, C₁-C₃ haloalkyl, C₁-C₃ alkoxy, C₃-C₅ cycloalkoxy, C₁-C₃ haloalkoxy, F, Cl, Br, OH, CN, NO₂, C(═O)OR^{a}, C(═O)N(R^{a})₂, S(═O)₂R^{b}, NR^{a}S(═O)₂R^{b}, C₄-C₁₀ heteroaryl, benzyl, and phenyl, wherein the benzyl and/or phenyl are each independently optionally substituted with at least one substituent selected from the group consisting of C₁-C₆ alkyl, C₁-C₆ alkoxy, C₁-C₃ haloalkyl, C₁-C₃ haloalkoxy, and halogen;
R³, if present, is selected from the group consisting of H and C₁-C₃ alkyl;
R⁴, if present, is selected from the group consisting of H and C₁-C₃ alkyl;
R^{A} and R^{B}, if present, are each independently selected from the group consisting of H, optionally substituted C₁-C₆ alkyl, optionally substituted benzyl, and C(═O)OR^{a},
   wherein each optional substituent in each of R^{A} and R^{B} is independently selected from the group consisting of C₁-C₆ alkyl and C₁-C₆ alkoxy; and
   wherein R^{A} and R^{B} may combine with the N to which they are bound to form a C₂-C₈ heterocycloalkyl;
R^{a1} and R^{a2}, if present, are each independently selected from the group consisting of H and C₁-C₃ alkyl;
T¹ and T², if present, are each independently phenyl optionally substituted with at least one selected from the group consisting of halogen, C₁-C₃ alkyl, C₁-C₃ haloalkyl, and C₁-C₃ haloalkoxy;
Ring G, if present, is selected from the group consisting of C₃-C₆ cycloalkyl and C₂-C₆ heterocycloalkyl, wherein ring G is optionally substituted with C₁-C₃ alkyl; and
Y¹ and Y², if present, are each independently selected from the group consisting of OR^{a} and N(R^{a})(R^{a}).

In certain embodiments, R¹ is

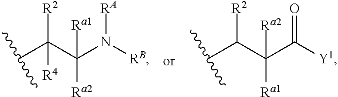

### and R² is

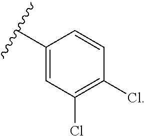 [SMILES: *c1ccc(Cl)c(Cl)c1]

In certain embodiments, R¹ is

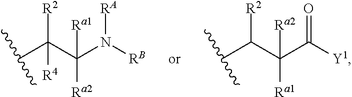

### and R² is

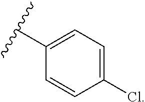 [SMILES: *c1ccc(Cl)cc1]

In certain embodiments, R¹ is

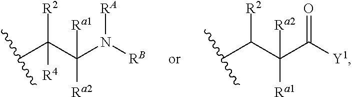

### and R² is

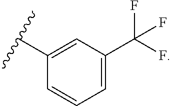 [SMILES: *c1cccc(C(F)(F)F)c1]

In certain embodiments, R¹

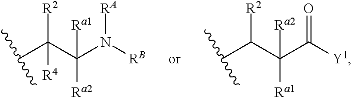

### and R² is

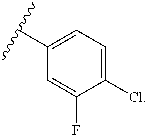 [SMILES: *c1ccc(Cl)c(F)c1]

In certain embodiments, R¹ is

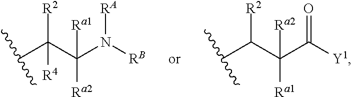

### and R² is

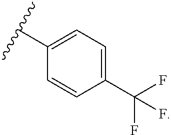 [SMILES: *c1ccc(C(F)(F)F)cc1]

In certain embodiments, R¹ is

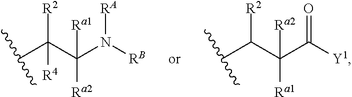

### and R² is

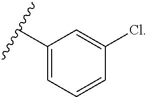 [SMILES: *c1cccc(Cl)c1]

In certain embodiments, R¹ is

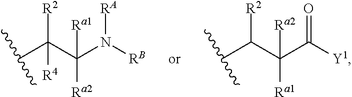

### and R² is

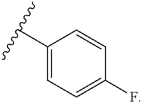 [SMILES: *c1ccc(F)cc1]

In certain embodiments, R¹ is

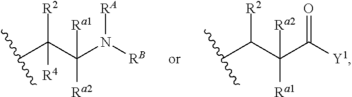

### and R² is

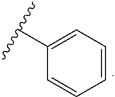 [SMILES: *c1ccccc1]

In certain embodiments, R¹ is

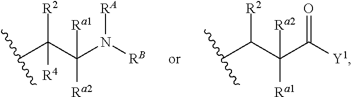

### R² is

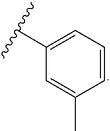 [SMILES: *c1cccc(C)c1]

In certain embodiments, R¹ is

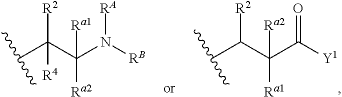

### and R² is

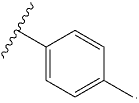 [SMILES: *c1ccc(C)cc1]

In certain embodiments, R¹ is

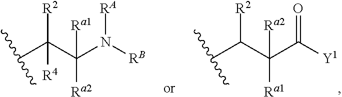

### and R² is

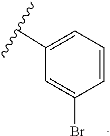 [SMILES: *c1cccc(Br)c1]

In certain embodiments, R¹ is

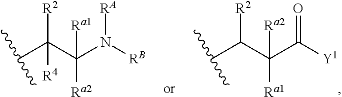

### and R² is

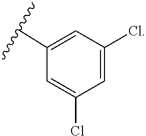 [SMILES: *c1cc(Cl)cc(Cl)c1]

In certain embodiments, R¹ is

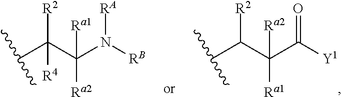

### and R² is

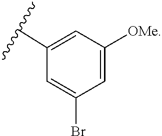 [SMILES: *c1cc(Br)cc(OC)c1]

In certain embodiments, R¹ is

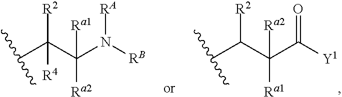

### and R² is

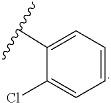 [SMILES: *c1ccccc1Cl]

In certain embodiments, R¹ is

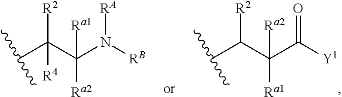

### and R² is

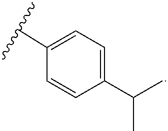 [SMILES: *c1ccc(C(C)C)cc1]

In certain embodiments, R¹ is

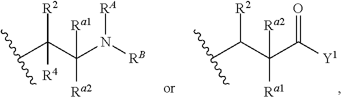

### and R² is

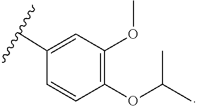 [SMILES: *c1ccc(OC(C)C)c(OC)c1]

In certain embodiments, R¹ is

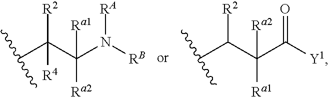

### and R² is

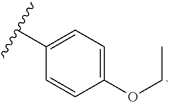 [SMILES: *c1ccc(OCC)cc1]

In certain embodiments, R¹ is

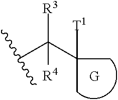

and R³ is H. In certain embodiments, R¹ is

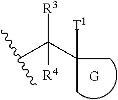

and R³ is CH₃.

In certain embodiments, R¹ is

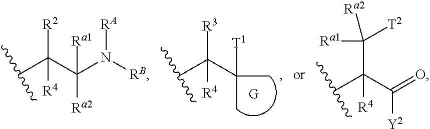

and R⁴ is H. In certain embodiments, R¹ is

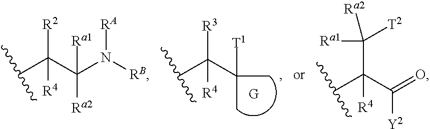

### and R⁴ is Me.

In certain embodiments, R¹ is

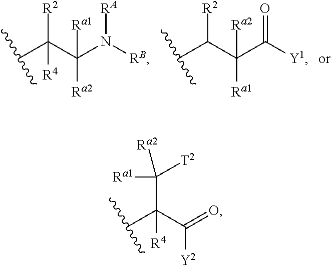

and R^{a1} is H. In certain embodiments, R¹ is

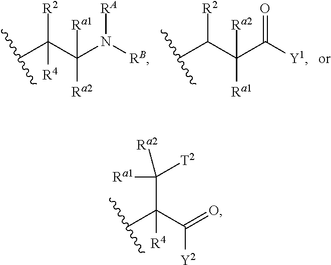

and R^{a1} is Me. In certain embodiments, R¹ is

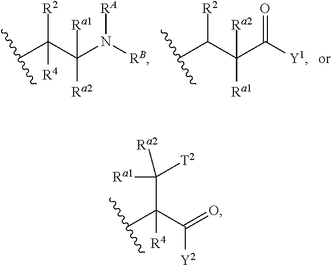

and R² is H. In certain embodiments, R¹ is

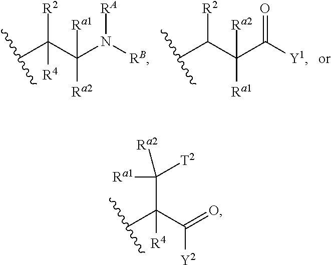

### and R^{a2} is Me.

In certain embodiments, T¹ is

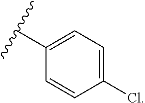 [SMILES: *c1ccc(Cl)cc1]

In certain embodiments, T¹ is

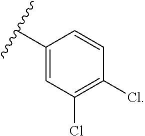 [SMILES: *c1ccc(Cl)c(Cl)c1]

In certain embodiments, T¹ is

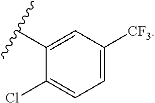 [SMILES: *c1cc(C(F)(F)F)ccc1Cl]

In certain embodiments, T¹ is

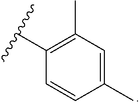 [SMILES: *c1ccc(C)cc1C]

In certain embodiments, T¹ is

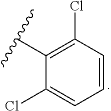 [SMILES: *c1c(Cl)cccc1Cl]

In certain embodiments, T¹ is

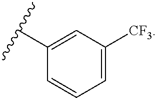 [SMILES: *c1cccc(C(F)(F)F)c1]

In certain embodiments, T¹ is

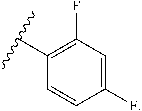 [SMILES: *c1ccc(F)cc1F]

In certain embodiments, T¹ is

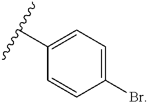 [SMILES: *c1ccc(Br)cc1]

In certain embodiments, T¹ is

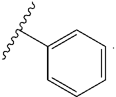 [SMILES: *c1ccccc1 ; "."]

In certain embodiments, T¹ is

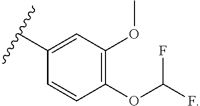 [SMILES: *c1ccc(OC(F)F)c(OC)c1]

In certain embodiments, T² is

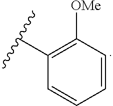 [SMILES: *c1ccccc1OC ; "."]

In certain embodiments, T² is

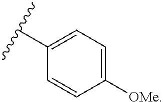 [SMILES: *c1ccc(OC)cc1]

In certain embodiments, T² is

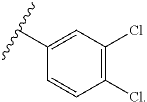 [SMILES: *c1ccc(Cl)c(Cl)c1]

In certain embodiments, T² is

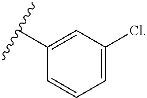 [SMILES: *c1cccc(Cl)c1]

In certain embodiments, T² is

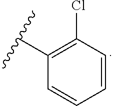 [SMILES: *c1ccccc1Cl ; "."]

In certain embodiments, T² is

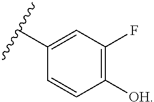 [SMILES: *c1ccc(O)c(F)c1]

In certain embodiments, T² is

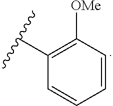 [SMILES: *c1ccccc1OC ; "."]

In certain embodiments, Y¹ is OH. In certain embodiments, Y¹ is OCH₃. In certain embodiments, Y¹ is OCH₂CH₃.

In certain embodiments, Y² is OCH₃. In certain embodiments, Y² is OCH₂CH₃.

In certain embodiments, ring G is

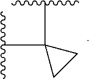 [SMILES: *C1(*)CC1 ; "."]

In certain embodiments, ring G is

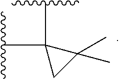 [SMILES: *C1(*)CC1(C)C ; "."]

In certain embodiments, ring G is

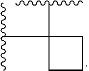 [SMILES: *C1(*)CCC1]

In certain embodiments, ring G is

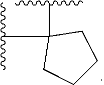 [SMILES: *C1(*)CCCC1]

In certain embodiments, ring G is

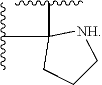 [SMILES: *C1(*)CCCN1]

In certain embodiments, ring G is

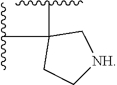 [SMILES: *C1(*)CCNC1]

In certain embodiments, ring G is

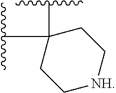 [SMILES: *C1(*)CCNCC1]

In certain embodiments, R^{A} is H. In certain embodiments, R^{A} is CH₃. In certain embodiments, R^{A} is C(═O)O(C(CH₃)₃). In certain embodiments, R^{A} is

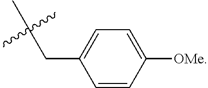 [SMILES: *Cc1ccc(OC)cc1]

In certain embodiments, R^{A} is

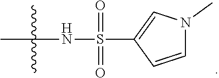 [SMILES: *NS(=O)(=O)c1ccn(C)c1]

In certain embodiments, R^{B} is H. In certain embodiments, R^{B} is CH₃. In certain embodiments, R^{B} is C(═O)O(C(CH₃)₃). In certain embodiments, R^{B} is

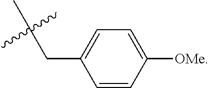 [SMILES: *Cc1ccc(OC)cc1]

In certain embodiments, R^{B} is

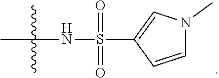 [SMILES: *NS(=O)(=O)c1ccn(C)c1]

In certain embodiments, R^{A} and R^{B} combine with the N atom to which they are bound to form

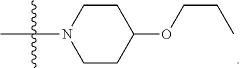 [SMILES: *N1CCC(OCCC)CC1]

In certain embodiments, R^{A} and R^{B} combine with the N atom to which they are bound to form

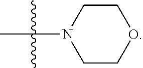 [SMILES: *N1CCOCC1]

In certain embodiments, R^{A} and R^{B} combine with the N atom to which they are bound to form

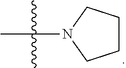 [SMILES: *N1CCCC1]

In certain embodiments, R^{A} and R^{B} combine with the N atom to which they are bound to form

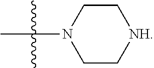 [SMILES: *N1CCNCC1]

In certain embodiments, R^{A} and R^{B} combine with the N atom to which they are bound to form

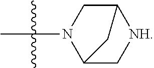 [SMILES: *N1CC2CC1CN2]

In certain embodiments, R^{A} and R^{B} combine with the N atom to which they are bound to form

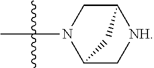 [SMILES: *N1C[C@@H]2C[C@H]1CN2]

In certain embodiments, R^{A} and R^{B} combine with the N atom to which they are bound to form

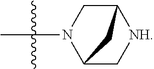 [SMILES: *N1C[C@H]2C[C@@H]1CN2]

In certain embodiments, R^{A} and R^{B} combine with the N atom to which they are bound to form

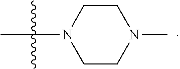 [SMILES: *N1CCN(C)CC1]

In certain embodiments, the compound of formula (I) is the compound of formula (II):

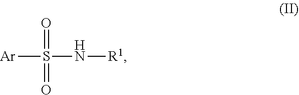

wherein:
Ar is phenyl, naphthyl, or pyridyl, any one of which is optionally substituted with at least one substituent selected from the group consisting of C₁-C₆ alkyl, C₁-C₆ haloalkyl, C₁-C₆ aminoalkyl, C₁-C₆ hydroxyalkyl, C₂-C₆ alkenyl, C₂-C₆ alkynyl, C₁-C₆ alkoxy, C₁-C₆ haloalkoxy, C₆-C₁₀ aryl, C₆-C₁₀ aryloxy, F, Cl, Br, I, OH, NH₂, CN, NO₂, C(═O)OR^{a}, and C(═O)N(R^{a})₂,
   wherein each C₆-C₁₀ aryl, C₄-C₁₀ heteroaryl, or C₆-C₁₀ aryloxy substituent is independently optionally substituted with at least one substituent selected from the group consisting of C₁-C₃ alkyl, C₁-C₃ haloalkyl, C₁-C₃ alkoxy, C₁-C₃ haloalkoxy, F, Cl, Br, I, OH, CN, NO₂, C(═O)OR^{a}, and C(═O)N(R^{a})(R^{a}),
   wherein two adjacent substituents of ring Z may optionally combine to provide a 4 to 8-membered fused ring;
R¹ is

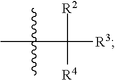

R² is selected from the group consisting of optionally substituted C₁-C₆ alkyl, optionally substituted C₁-C₆ haloalkyl, optionally substituted C₁-C₆ aminoalkyl, optionally substituted C₁-C₆ hydroxyalkyl, optionally substituted C₂-C₆ alkenyl, optionally substituted C₂-C₆ alkenyl, optionally substituted C₂-C₆ alkynyl, optionally substituted C₁-C₆ alkoxy, optionally substituted C₁-C₆ haloalkoxy, optionally substituted C₃-C₁₂ cycloalkyl, optionally substituted C₂-C₁₂ heterocycloalkyl, optionally substituted C₆-C₁₀ aryl, and optionally substituted C₄-C₁₀ heteroaryl,
   wherein each optional substituent in R² is independently selected from the group consisting of C₁-C₃ alkyl, C₁-C₃ haloalkyl, C₁-C₃ alkoxy, C₃-C₅ cycloalkoxy, C₃-C₁₂ heterocycloalkyl, C₁-C₃ haloalkoxy, F, Cl, Br, OR, CN, NO₂, C(═O)OR^{a}, C(═O)N(R^{a})₂, S(═O)₂R^{b}, NR^{a}S(═O)₂R^{b}, C₄-C₁₀ heteroaryl, benzyl, and phenyl, wherein the benzyl or phenyl is optionally substituted with at least one substituent selected from the group consisting of C₁-C₆ alkyl, C₁-C₆ alkoxy, C₁-C₃ haloalkyl, C₁-C₃ haloalkoxy, and halogen;
R³ is selected from the group consisting of H, C(═O)R^{a}, C(═O)OR^{a}, C(═O)N(R^{a})₂, NR^{a}C(═O)OR, optionally substituted C₁-C₆ alkyl, optionally substituted C₁-C₆ alkoxy, optionally substituted C₁-C₆ haloalkyl, optionally substituted C₁-C₆ aminoalkyl, optionally substituted C₁-C₆ hydroxyalkyl, optionally substituted C₃-C₁₂ cycloalkyl, and optionally substituted C₂-C₁₂ heterocycloalkyl,
   wherein each optional substituent in R³ is selected from the group consisting of C₁-C₃ alkyl, C₂-C₁₂ heterocycloalkyl, C₁-C₃ haloalkyl, C₁-C₃ alkoxy, C₁-C₃ haloalkoxy, benzyl, C₆-C₁₀ aryl, C₄-C₁₀ heteroaryl, F, Cl, Br, OH, CN, NO₂, C(═O)OR^{a}, C(═O)N(R^{a})(R^{b}), NR^{a}C(═O)OR^{a}, S(═O)₂R^{b}, and NR^{a}S(═O)₂R^{b},
R⁴ is H or C₁-C₆ alkyl;
each occurrence of R^{a} is independently selected from the group consisting of H, C₁-C₆ alkyl, C₃-C₆ cycloalkyl, benzyl, and C₆-C₁₀ aryl;
each occurrence of R^{b} is independently selected from the group consisting of H, optionally substituted C₁-C₆ alkyl, optionally substituted C₂-C₁₂ heterocycloalkyl, optionally substituted benzyl, optionally substituted C₂-C₅ heteroaryl, optionally substituted phenyl, and optionally substituted naphthyl,
   wherein each optional substituent in R^{b} is independently selected from the group consisting of C₁-C₃ alkyl, C₁-C₃ haloalkyl, C₁-C₃ alkoxy, C₁-C₃ haloalkoxy, F, Cl, Br, I, OH, CN, NO₂, C(═O)OR^{a}, and C(═O)N(R^{a})₂,
wherein no more than one of R² and R³ is CH₂OH;
wherein if R³ is H and R⁴ is H, R³ is CH₃ and R⁴ is H, or R³ is H and R⁴ is CH₃, then one of the following applies:
   (a) R² comprises at least one substituent selected from the group consisting of C₆-C₁₀ aryl and C₄-C₁₀ heteroaryl, wherein the aryl and/or heteroaryl are each independently substituted with at least one substituent selected from the group consisting of OR^{b}, N(R^{a})(R^{a}), C₁-C₆ alkyl, C₁-C₃ haloalkyl, C₁-C₃ haloalkoxy, C₂-C₁₂ heterocycloalkyl, F, Cl, Br, and S(═O)₂R^{b};
   (b) R² comprises at least one substituent selected from the group consisting of C₃-C₆ cycloalkyl and C₂-C₁₂ heterocycloalkyl, and at least one substituent selected from the group consisting of C₆-C₁₀ aryl and C₄-C₁₀ heteroaryl;
wherein if L is a bond, R³ is H, and R⁴ is H, and A¹ is

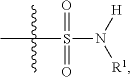

then the optionally substituted C₂-C₁₂ heterocycloalkyl in R² comprises no more than one phenyl substituted with at least one halogen;
wherein if Z is optionally substituted phenyl, L is a bond, A¹ is

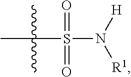

and R² is optionally substituted C₆-C₁₀ aryl, then one of the following applies:
(a) the phenyl in Z is substituted with at least one substituent selected from the group consisting of C₁-C₆ haloalkyl, C₁-C₆ aminoalkyl, C₁-C₆ hydroxyalkyl, C₂-C₆ alkenyl, C₂-C₆ alkynyl, C₁-C₆ alkoxy, C₁-C₆ haloalkoxy, C₆-C₁₀ aryl, C₃-C₆ heterocycloalkyl, C₄-C₁₀ heteroaryl, C₆-C₁₀ aryloxy, C₄-C₁₀ heteroaryloxy F, Cl, Br, I, OH, CN, N(R^{a})(R^{b}), NO₂, C(═O)OR^{a}, and C(═O)N(R^{a})(R^{b}), and the C₆-C₁₀ aryl in R² is substituted with at least one substituent selected from the group consisting of C₁-C₃ haloalkyl, C₁-C₃ alkoxy, C₃-C₅ cycloalkoxy, C₃-C₁₂ heterocycloalkyl, C₁-C₃ haloalkoxy, F, Cl, Br, OR, CN, NO₂, C(═O)OR^{a}, C(═O)N(R^{a})₂, S(═O)₂R^{b}, NR^{a}S(═O)₂R^{b}, C₄-C₁₀ heteroaryl, and benzyl;
(b) the phenyl in Z is substituted with at least one substituent, and the C₆-C₁₀ aryl in R² is substituted with at least two substituents; or
(c) the phenyl in Z is substituted with at least one trifluoromethoxy substituent, the C₆-C₁₀ aryl in R² is substituted with at least one substituent, and R³ is optionally substituted C₁-C₆ aminoalkyl;
wherein if Z is 4-trifluoromethoxyphenyl, L is a bond, and A¹ is

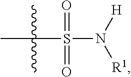

then each of the following apply:
(a) if R³ and R⁴ are each independently H, then R² is not selected from the group consisting of phenyl, 2-difluoromethoxyphenyl, 2-methylphenyl, 2-cyanophenyl, 2-methoxyphenyl, 2-trifluoromethoxyphenyl, 2-chlorophenyl, 2-fluorophenyl, 2-trifluoromethylphenyl, 3-methylphenyl, 3-methoxyphenyl, 3-bromophenyl, 3-chlorophenyl, 3-trifluoromethoxyphenyl, 3-trifluoromethylphenyl, 4-isobutyramido-phenyl, 4-tert-butylphenyl, 4-methoxyphenyl, 4-chlorophenyl, 4-bromophenyl, 4-carboxyphenyl, 4-trifluoromethylphenyl, 4-methylphenyl, 4-fluorophenyl, 2-fluoro-3-trifluoromethylphenyl, 2,3-dimethylphenyl, 2,3-dichlorophenyl, 2-methyl-4-chlorophenyl, 2-chloro-4-fluorophenyl, 2,4-dimethoxyphenyl, 2-methoxy-4-(2-methoxy-5-fluorophenyl)phenyl, 2-methoxy-4-chlorophenyl, 2-fluoro-4-trifluoromethylphenyl, 2,6-difluorophenyl, 2-chloro-6-methylphenyl, 2,6-dimethoxyphenyl, 3,4-ethylenedioxyphenyl, 3-fluoro-4-trifluoromethylphenyl, 3,4-difluorophenyl, 3-fluoro-4-methylphenyl, 3-methoxy-4-ethoxyphenyl, 3,4-dimethoxyphenyl, 3-chloro-4-methylphenyl, 3-methyl-4-fluorophenyl, 3-chloro-4-fluorophenyl, 3,5-difluorophenyl, 3,5-dichlorophenyl, 3,5-dimethoxyphenyl, 3,5-dimethylphenyl, 3,6-dichlorophenyl, 3,6-difluorophenyl, 3,6-dimethoxyphenyl, 3-chloro-6-methylphenyl, 2,3,4-trifluorophenyl, 3,4,5-trifluorophenyl, 2,6-difluoro-3-chlorophenyl, 1-((5-ethanolyl)thiophen-2-yl)cyclopentyl, and 1-(thiophen-2-yl)cyclopentyl;
(b) if R³ is H and R⁴ is methyl or R³ is methyl and R⁴ is H, then R² is not selected from the group consisting of phenyl, naphthyl, 3-methoxyphenyl, 2,4-difluorophenyl, 3,4-methylenedioxyphenyl, 3,4-(1,2-ethylenedioxy)phenyl, 3-fluoro-4-methoxyphenyl, 3,6-dimethoxyphenyl, 2-methylphenyl, 2-methoxyphenyl, 4-methylphenyl, 4-bromophenyl, 4-fluorophenyl, 4-methoxyphenyl, 4-ethoxyphenyl, 4-(methanesulfonyl)phenyl, 4-(3-imidazolyl)phenyl, and 4-(2-(1,2,4-triazol)-yl)phenyl;
(c) if R³ is H and R⁴ is ethyl or R³ is selected from the group consisting of ethyl and cyclopropyl and R⁴ is H, then R² is not selected from the group consisting of phenyl and 4-methoxyphenyl;
(d) if R³ is selected from the group consisting of hydroxymethyl, cyano, 1-(1,2,4-triazol)-yl, and 1-(1,2,5-triazol)-yl, and R⁴ is H, then R² is not phenyl;
(e) if R³ is CH₂C(═O)OH and R⁴ is H, then R² is not selected from the group consisting of phenyl, 2-chlorophenyl, 4-methylphenyl, 4-fluorophenyl, 4-chlorophenyl, 4-tert-butylphenyl, 4-methoxyphenyl, and 3,4-dimethoxyphenyl;
(f) if R³ is CH₂C(═O)OCH₃ and R⁴ is H, then R² is not selected from the group consisting of cyclohexyl, phenyl, 4-chlorophenyl, 4-tert-butylphenyl, 4-methylphenyl, 4-bromophenyl, 4-fluorophenyl, and 3-bromophenyl;
(g) if R³ is CH₂C(═O)OCH₂CH₃ and R⁴ is H, then R² is not selected from the group consisting of phenyl, 2-chlorophenyl, 4-fluorophenyl, and 4-bromophenyl;
(h) if R³ is CH₂(C═O)OCH₂Ph and R⁴ is H, then R² is not 2-chlorophenyl;
(i) if R³ is CH₂(C═O)OCH(CH₃)₂ and R⁴ is H, then R² is not 2-nitrophenyl; and
(j) if R³ is selected from the group consisting of C(═O)NH₂, C(═O)OCH₃, and C(═O)OCH₂CH₃ and R⁴ is H, then R² is not phenyl; and
wherein if Z is 4-difluoromethoxyphenyl, L is a bond, and A¹ is

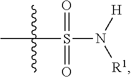

then each of the following apply:
(a) if R³ is CH₂(C═O)OH and R⁴ is H, then R² is not 2-chlorophenyl;
(b) if R³ is CH₂(C═O)OCH₃ and R⁴ is H, then R² is not selected from the group consisting of cyclohexyl, 4-chlorophenyl, and 4-bromophenyl;
(c) if R³ is CH₂(C═O)OCH₂CH₃ and R⁴ is H, then R² is not selected from the group consisting of phenyl, 2-chlorophenyl, and 4-bromophenyl; and
(d) if R³ is CH₂(C═O)OCH₂Ph and R⁴ is H, then R² is not 2-chlorophenyl.

In certain embodiments, Ar is

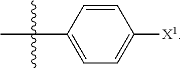

In certain embodiments, Ar is

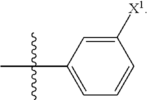

In certain embodiments, Ar is

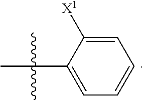

In certain embodiments, Ar is

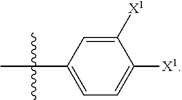

In certain embodiments, Ar is

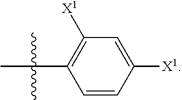

In certain embodiments, Ar is

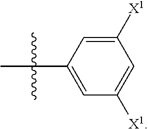

In certain embodiments, Ar is

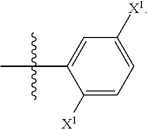

In certain embodiments, Ar is

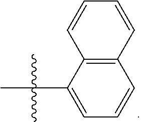 [SMILES: *c1cccc2ccccc12]

In certain embodiments, Ar is

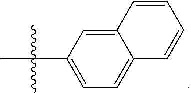 [SMILES: *c1ccc2ccccc2c1]

In certain embodiments, Ar is

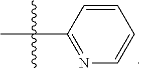 [SMILES: *c1ccccn1]

In certain embodiments, Ar is

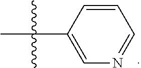 [SMILES: *c1cccnc1]

In certain embodiments, Ar is

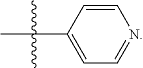 [SMILES: *c1ccncc1]

In certain embodiments, X¹ is methyl. In certain embodiments, X¹ is ethyl. In certain embodiments, X¹ is i-propyl. In certain embodiments, X¹ is n-propyl. In certain embodiments, X¹ is n-butyl. In certain embodiments, X¹ is n-pentyl. In certain embodiments, X¹ is Cl. In certain embodiments, X¹ is F. In certain embodiments, X¹ is OCH₂CH₃. In certain embodiments, X¹ is OCH₂CH₂CH₃. In certain embodiments, X¹ is OCH(CH₃)₂. In certain embodiments, X¹ is CF₃. In certain embodiments, X¹ is OCF₃. In certain embodiments, X¹ is OCHF₂. In certain embodiments, X¹ is NO₂. In certain embodiments, X¹ is C(═O)OR^{a}. In certain embodiments, X¹ is C(═O)NHR^{a}. In certain embodiments, X¹ is NH₂. In certain embodiments, X¹ is CN. In certain embodiments, X¹ is

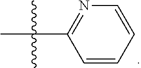 [SMILES: *c1ccccn1]

In certain embodiments, X¹ is

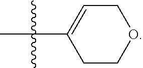 [SMILES: *C1=CCOCC1]

In certain embodiments, X is

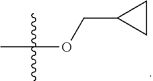 [SMILES: *OCC1CC1]

In certain embodiments, X¹ is

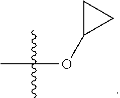 [SMILES: *OC1CC1]

In certain embodiments, X¹ is

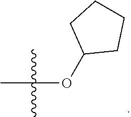 [SMILES: *OC1CCCC1]

In certain embodiments, X¹ is

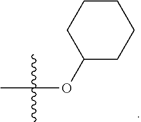 [SMILES: *OC1CCCCC1]

In certain embodiments, X¹ is

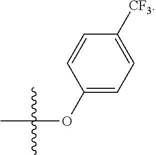 [SMILES: *Oc1ccc(C(F)(F)F)cc1]

In certain embodiments, X¹ is

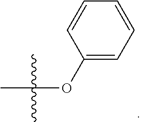 [SMILES: *Oc1ccccc1]

In certain embodiments, X¹ is

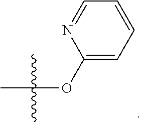 [SMILES: *Oc1ccccn1]

In certain embodiments, X¹ is

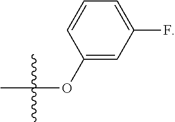 [SMILES: *Oc1cccc(F)c1]

In certain embodiments, X¹ is

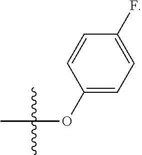 [SMILES: *Oc1ccc(F)cc1]

In certain embodiments, X¹ is

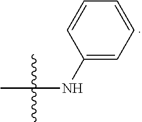 [SMILES: *Nc1ccccc1]

In certain embodiments, X¹ is

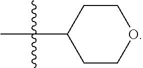 [SMILES: *C1CCOCC1]

In certain embodiments, X¹ is

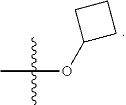 [SMILES: *OC1CCC1]

In certain embodiments, X¹ is

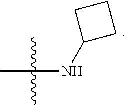 [SMILES: *NC1CCC1]

In certain embodiments, X¹ is

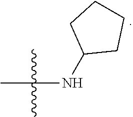 [SMILES: *NC1CCCC1]

In certain embodiments, X is

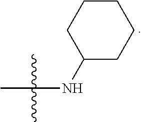 [SMILES: *NC1CCCCC1]

In certain embodiments, X¹ is

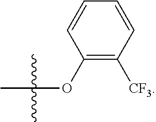 [SMILES: *Oc1ccccc1C(F)(F)F]

In certain embodiments, Ar is

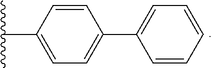 [SMILES: *c1ccc(-c2ccccc2)cc1]

In certain embodiments, Ar is

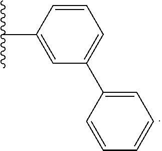 [SMILES: *c1cccc(-c2ccccc2)c1]

In certain embodiments, Ar is

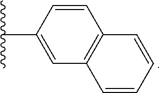 [SMILES: *c1ccc2ccccc2c1]

In certain embodiments, Ar is

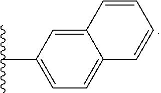 [SMILES: *c1ccc2ccccc2c1]

In certain embodiments, Ar is

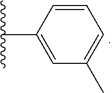 [SMILES: *c1cccc(C)c1]

In certain embodiments, Ar is

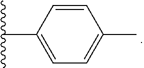 [SMILES: *c1ccc(C)cc1]

In certain embodiments, Ar is

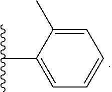 [SMILES: *c1ccccc1C]

In certain embodiments, Ar is

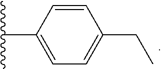 [SMILES: *c1ccc(CC)cc1]

In certain embodiments, Ar is

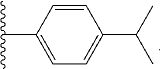 [SMILES: *c1ccc(C(C)C)cc1]

In certain embodiments, Ar is

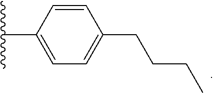 [SMILES: *c1ccc(CCCC)cc1]

In certain embodiments, Ar is

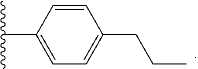 [SMILES: *c1ccc(CCC)cc1]

In certain embodiments, Ar is

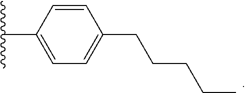 [SMILES: *c1ccc(CCCCC)cc1]

In certain embodiments, Ar is

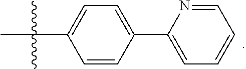 [SMILES: *c1ccc(-c2ccccn2)cc1]

In certain embodiments, Ar is

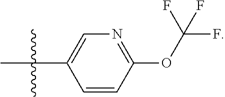 [SMILES: *c1ccc(OC(F)(F)F)nc1]

In certain embodiments, Ar is

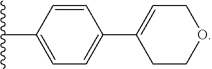 [SMILES: *c1ccc(C2=CCOCC2)cc1]

In certain embodiments, Ar is

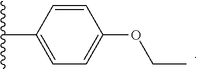 [SMILES: *c1ccc(OCC)cc1]

In certain embodiments, Ar is

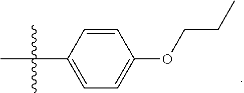 [SMILES: *c1ccc(OCCC)cc1]

In certain embodiments, Ar is

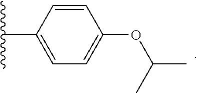 [SMILES: *c1ccc(OC(C)C)cc1]

In certain embodiments, Ar is

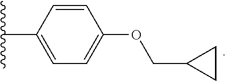 [SMILES: *c1ccc(OCC2CC2)cc1]

In certain embodiments, Ar is

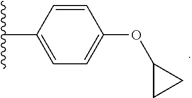 [SMILES: *c1ccc(OC2CC2)cc1]

In certain embodiments, Ar is

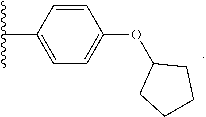 [SMILES: *c1ccc(OC2CCCC2)cc1]

In certain embodiments, Ar is

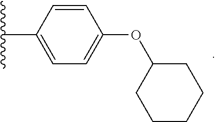 [SMILES: *c1ccc(OC2CCCCC2)cc1]

In certain embodiments, Ar is

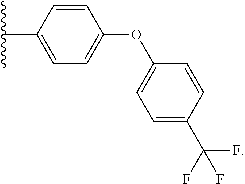 [SMILES: *c1ccc(Oc2ccc(C(F)(F)F)cc2)cc1]

In certain embodiments, Ar is

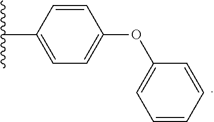 [SMILES: *c1ccc(Oc2ccccc2)cc1]

In certain embodiments, Ar is

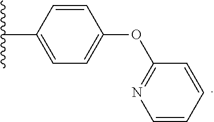 [SMILES: *c1ccc(Oc2ccccn2)cc1]

In certain embodiments, Ar is

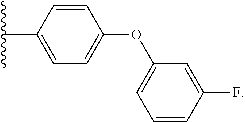 [SMILES: *c1ccc(Oc2cccc(F)c2)cc1]

In certain embodiments, Ar is

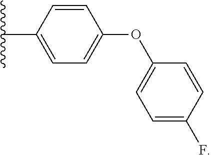 [SMILES: *c1ccc(Oc2ccc(F)cc2)cc1]

In certain embodiments, Ar is

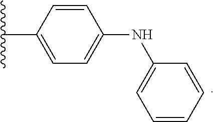 [SMILES: *c1ccc(Nc2ccccc2)cc1]

In certain embodiments, Ar is

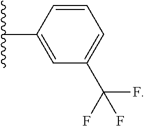 [SMILES: *c1cccc(C(F)(F)F)c1]

In certain embodiments, Ar is

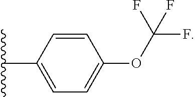 [SMILES: *c1ccc(OC(F)(F)F)cc1]

In certain embodiments, Ar is

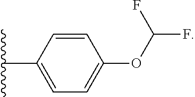 [SMILES: *c1ccc(OC(F)F)cc1]

In certain embodiments, Ar is

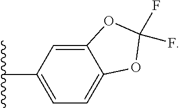 [SMILES: *c1ccc2c(c1)OC(F)(F)O2]

In certain embodiments, Ar is

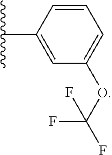 [SMILES: *c1cccc(OC(F)(F)F)c1]

In certain embodiments, Ar is

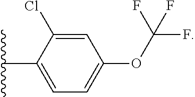 [SMILES: *c1ccc(OC(F)(F)F)cc1Cl]

In certain embodiments, Ar is

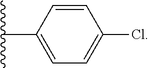 [SMILES: *c1ccc(Cl)cc1]

In certain embodiments, Ar is

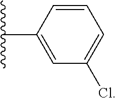 [SMILES: *c1cccc(Cl)c1]

In certain embodiments, Ar is

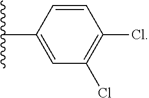 [SMILES: *c1ccc(Cl)c(Cl)c1]

In certain embodiments, Ar is

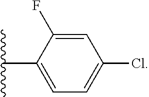 [SMILES: *c1ccc(Cl)cc1F]

In certain embodiments, Ar is

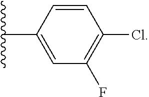 [SMILES: *c1ccc(Cl)c(F)c1]

In certain embodiments, Ar is

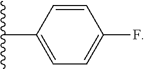 [SMILES: *c1ccc(F)cc1]

In certain embodiments, Ar is

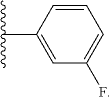 [SMILES: *c1cccc(F)c1]

In certain embodiments, Ar is

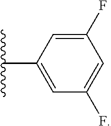 [SMILES: *c1cc(F)cc(F)c1]

In certain embodiments, Ar is

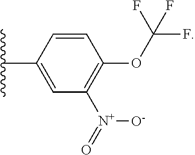 [SMILES: *c1ccc(OC(F)(F)F)c([N+](=O)[O-])c1]

In certain embodiments, Ar is

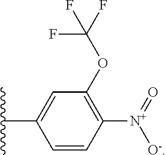

In certain embodiments, Ar is

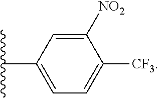 [SMILES: *c1ccc(C(F)(F)F)c([N+](=O)[O-])c1]

In certain embodiments, Ar is

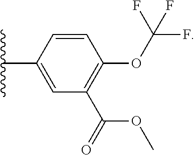 [SMILES: *c1ccc(OC(F)(F)F)c(C(=O)OC)c1]

In certain embodiments, Ar is

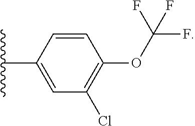 [SMILES: *c1ccc(OC(F)(F)F)c(Cl)c1]

In certain embodiments, Ar is

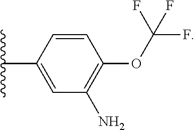 [SMILES: *c1ccc(OC(F)(F)F)c(N)c1]

In certain embodiments, Ar is

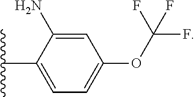 [SMILES: *c1ccc(OC(F)(F)F)cc1N]

In certain embodiments, Ar is

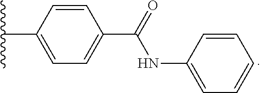 [SMILES: *c1ccc(C(=O)Nc2ccccc2)cc1]

In certain embodiments, Ar is

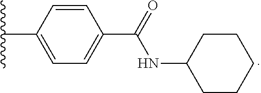 [SMILES: *c1ccc(C(=O)NC2CCCCC2)cc1]

In certain embodiments, Ar is CN

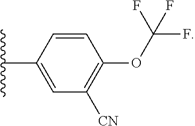 [SMILES: *c1ccc(OC(F)(F)F)c(C#N)c1]

In certain embodiments, Ar is

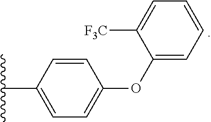 [SMILES: *c1ccc(Oc2ccccc2C(F)(F)F)cc1]

In certain embodiments, Ar is

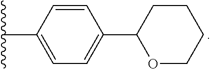 [SMILES: *c1ccc(C2CCCCO2)cc1]

In certain embodiments, Ar is

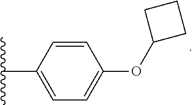 [SMILES: *c1ccc(OC2CCC2)cc1]

In certain embodiments, Ar is

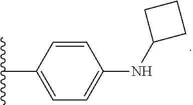 [SMILES: *c1ccc(NC2CCC2)cc1]

In certain embodiments, Ar is

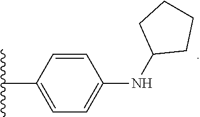 [SMILES: *c1ccc(NC2CCCC2)cc1]

In certain embodiments,

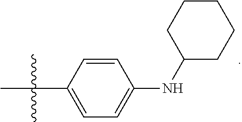 [SMILES: *c1ccc(NC2CCCCC2)cc1]

In certain embodiments, R² is

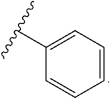 [SMILES: *c1ccccc1]

In certain embodiments, R² is

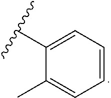 [SMILES: *c1ccccc1C]

In certain embodiments, R² is

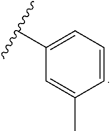 [SMILES: *c1cccc(C)c1]

In certain embodiments, R² is

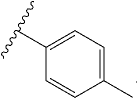 [SMILES: *c1ccc(C)cc1]

In certain embodiments, R² is

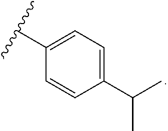 [SMILES: *c1ccc(C(C)C)cc1]

In certain embodiments, R² is

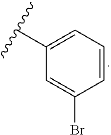 [SMILES: *c1cccc(Br)c1]

In certain embodiments, R² is

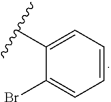 [SMILES: *c1ccccc1Br]

In certain embodiments, R² is

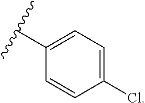 [SMILES: *c1ccc(Cl)cc1]

In certain embodiments, R² is

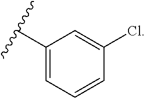 [SMILES: *c1cccc(Cl)c1]

In certain embodiments, R² is

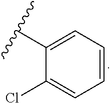 [SMILES: *c1ccccc1Cl]

In certain embodiments, R² is

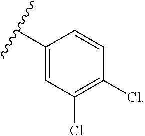 [SMILES: *c1ccc(Cl)c(Cl)c1]

In certain embodiments, R² is

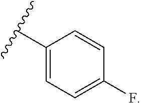 [SMILES: *c1ccc(F)cc1]

In certain embodiments, R² is

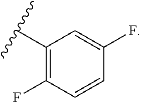 [SMILES: *c1cc(F)ccc1F]

In certain embodiments, R² is

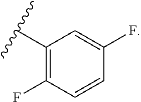 [SMILES: *c1cc(F)ccc1F]

In certain embodiments, R² is

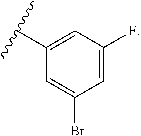 [SMILES: *c1cc(F)cc(Br)c1]

In certain embodiments, R² is

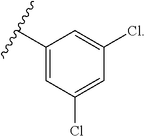 [SMILES: *c1cc(Cl)cc(Cl)c1]

In certain embodiments, R² is

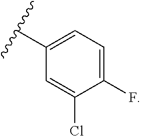 [SMILES: *c1ccc(F)c(Cl)c1]

In certain embodiments, R² is

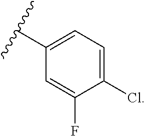 [SMILES: *c1ccc(Cl)c(F)c1]

In certain embodiments, R² is

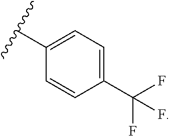 [SMILES: *c1ccc(C(F)(F)F)cc1]

In certain embodiments, R² is

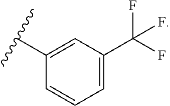 [SMILES: *c1cccc(C(F)(F)F)c1]

In certain embodiments, R² is

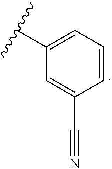 [SMILES: *c1cccc(C#N)c1]

In certain embodiments, R² is

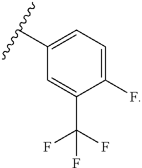 [SMILES: *c1ccc(F)c(C(F)(F)F)c1]

In certain embodiments, R² is

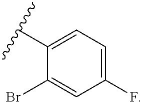 [SMILES: *c1ccc(F)cc1Br]

In certain embodiments, R² is

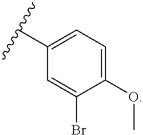 [SMILES: *c1ccc(OC)c(Br)c1]

In certain embodiments, R² is

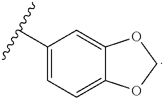 [SMILES: *c1ccc2c(c1)OCO2]

In certain embodiments, R² is

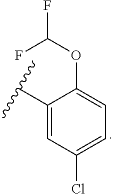 [SMILES: *c1cc(Cl)ccc1OC(F)F]

In certain embodiments, R² is

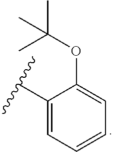 [SMILES: *c1ccccc1OC(C)(C)C]

In certain embodiments, R² is

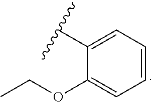 [SMILES: *c1ccccc1OCC]

In certain embodiments, R² is

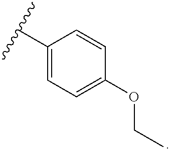 [SMILES: *c1ccc(OCC)cc1]

In certain embodiments, R² is

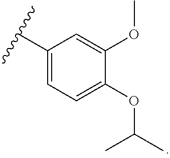 [SMILES: *c1ccc(OC(C)C)c(OC)c1]

In certain embodiments, R² is

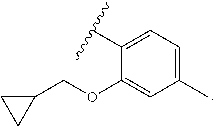 [SMILES: *c1ccc(C)cc1OCC1CC1]

In certain embodiments, R² is

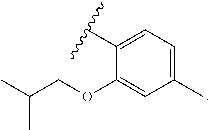 [SMILES: *c1ccc(C)cc1OCC(C)C]

In certain embodiments, R² is

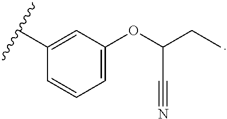 [SMILES: *c1cccc(OC(C#N)CC)c1]

In certain embodiments, R² is

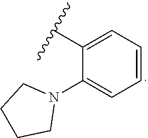 [SMILES: *c1ccccc1N1CCCC1]

In certain embodiments, R² is

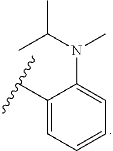 [SMILES: *c1ccccc1N(C)C(C)C]

In certain embodiments, R² is

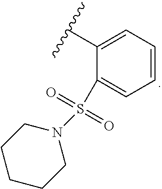 [SMILES: *c1ccccc1S(=O)(=O)N1CCCCC1]

In certain embodiments, R² is

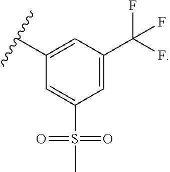 [SMILES: *c1cc(C(F)(F)F)cc(S(C)(=O)=O)c1]

In certain embodiments, R² is

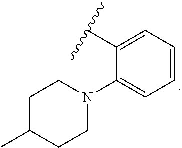 [SMILES: *c1ccccc1N1CCC(C)CC1]

In certain embodiments, R² is

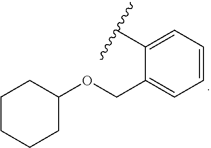 [SMILES: *c1ccccc1COC1CCCCC1]

In certain embodiments, R² is

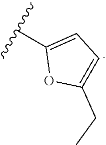 [SMILES: *c1ccc(CC)o1]

In certain embodiments, R² is

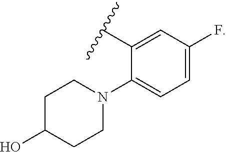 [SMILES: *c1cc(F)ccc1N1CCC(O)CC1]

In certain embodiments, R² is

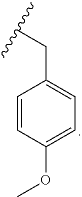 [SMILES: *Cc1ccc(OC)cc1]

In certain embodiments, R² is

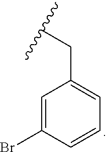 [SMILES: *Cc1cccc(Br)c1]

In certain embodiments, R² is

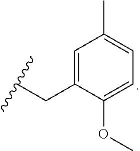 [SMILES: *Cc1cc(C)ccc1OC]

In certain embodiments, R² is

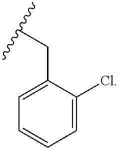 [SMILES: *Cc1ccccc1Cl]

In certain embodiments, R² is

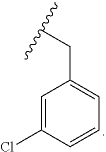 [SMILES: *Cc1cccc(Cl)c1]

In certain embodiments, R² is

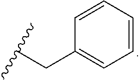 [SMILES: *Cc1ccccc1]

In certain embodiments, R² is

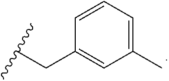 [SMILES: *Cc1cccc(C)c1]

In certain embodiments, R² is

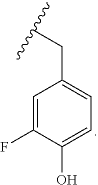 [SMILES: *Cc1ccc(O)c(F)c1]

In certain embodiments, R² is

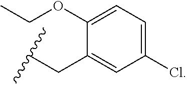 [SMILES: *Cc1cc(Cl)ccc1OCC]

In certain embodiments, R² is

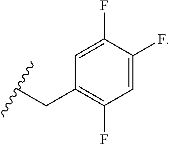 [SMILES: *Cc1cc(F)c(F)cc1F]

In certain embodiments, R² is

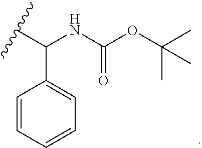 [SMILES: *C(NC(=O)OC(C)(C)C)c1ccccc1]

In certain embodiments, R² is

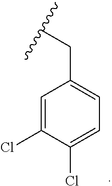 [SMILES: *Cc1ccc(Cl)c(Cl)c1]

In certain embodiments, R² is

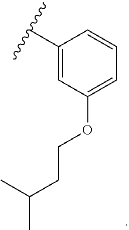 [SMILES: *c1cccc(OCCC(C)C)c1]

In certain embodiments, R² is

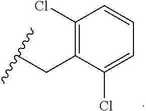 [SMILES: *Cc1c(Cl)cccc1Cl]

In certain embodiments, R² is

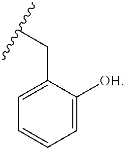 [SMILES: *Cc1ccccc1O]

In certain embodiments, R² is

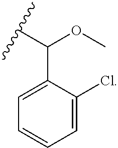 [SMILES: *C(OC)c1ccccc1Cl]

In certain embodiments, R² is

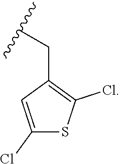 [SMILES: *Cc1cc(Cl)sc1Cl]

In certain embodiments, R² is

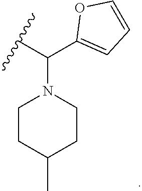 [SMILES: *C(c1ccco1)N1CCC(C)CC1]

In certain embodiments, R² is

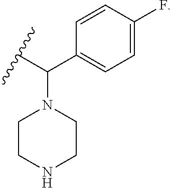 [SMILES: *C(c1ccc(F)cc1)N1CCNCC1]

In certain embodiments, R² is

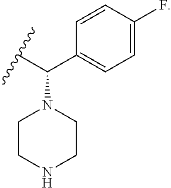 [SMILES: *[C@H](c1ccc(F)cc1)N1CCNCC1]

In certain embodiments, R² is

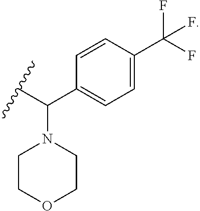 [SMILES: *C(c1ccc(C(F)(F)F)cc1)N1CCOCC1]

In certain embodiments, R² is

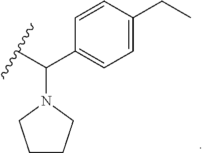 [SMILES: *C(c1ccc(CC)cc1)N1CCCC1]

In certain embodiments, R² is

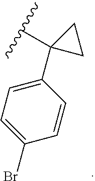 [SMILES: *C1(c2ccc(Br)cc2)CC1]

In certain embodiments, R² is

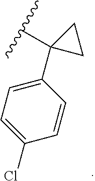 [SMILES: *C1(c2ccc(Cl)cc2)CC1]

In certain embodiments, R² is

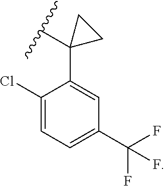 [SMILES: *C1(c2cc(C(F)(F)F)ccc2Cl)CC1]

In certain embodiments, R² is

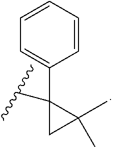 [SMILES: *C1(c2ccccc2)CC1(C)C]

In certain embodiments, R² is

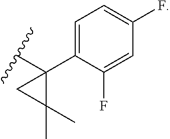 [SMILES: *C1(c2ccc(F)cc2F)CC1(C)C]

In certain embodiments, R² is

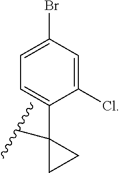 [SMILES: *C1(c2ccc(Br)cc2Cl)CC1]

In certain embodiments, R² is

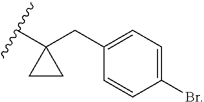 [SMILES: *C1(Cc2ccc(Br)cc2)CC1]

In certain embodiments, R² is

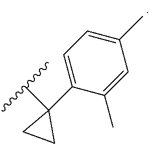 [SMILES: *C1(c2ccc(C)cc2C)CC1]

In certain embodiments, R² is

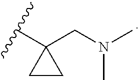 [SMILES: *C1(CN(C)C)CC1]

In certain embodiments, R² is

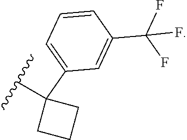 [SMILES: *C1(c2cccc(C(F)(F)F)c2)CCC1]

In certain embodiments, R² is

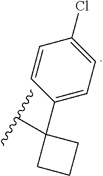 [SMILES: *C1(c2ccc(Cl)cc2)CCC1]

In certain embodiments, R² is

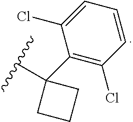 [SMILES: *C1(c2c(Cl)cccc2Cl)CCC1]

In certain embodiments, R² is

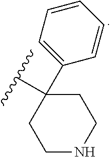 [SMILES: *C1(c2ccccc2)CCNCC1]

In certain embodiments, R² is

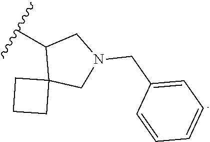 [SMILES: *C1CN(Cc2ccccc2)CC12CCC2]

In certain embodiments, R² is

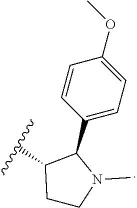 [SMILES: *[C@H]1CCN(C)[C@@H]1c1ccc(OC)cc1]

In certain embodiments, R² is

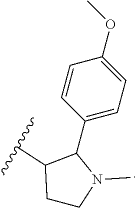 [SMILES: *C1CCN(C)C1c1ccc(OC)cc1]

In certain embodiments, R² is

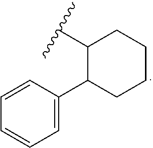 [SMILES: *C1CCCCC1c1ccccc1]

In certain embodiments, R² is

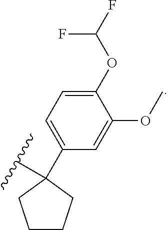 [SMILES: *C1(c2ccc(OC(F)F)c(OC)c2)CCCC1]

In certain embodiments, R² is

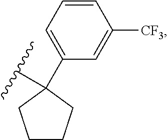 [SMILES: *C1(c2cccc(C(F)(F)F)c2)CCCC1]

In certain embodiments, R² is

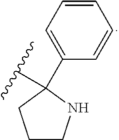 [SMILES: *C1(c2ccccc2)CCCN1]

In certain embodiments, R² is

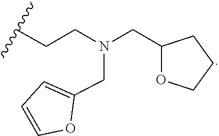 [SMILES: *CCN(Cc1ccco1)CC1CCCO1]

In certain embodiments, R² is

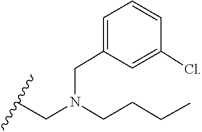 [SMILES: *CN(CCCC)Cc1cccc(Cl)c1]

In certain embodiments R² is

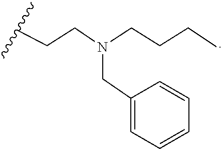 [SMILES: *CCN(CCCC)Cc1ccccc1]

In certain embodiments, R² is

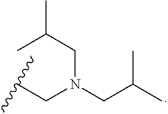 [SMILES: *CN(CC(C)C)CC(C)C]

In certain embodiments, R² is

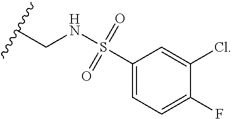 [SMILES: *CNS(=O)(=O)c1ccc(F)c(Cl)c1]

In certain embodiments R² is

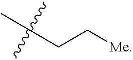 [SMILES: *CCC]

In certain embodiments, R² is

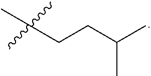 [SMILES: *CCC(C)C]

In certain embodiments, R² is

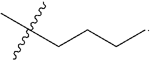 [SMILES: *CCCC]

In certain embodiments, R² is

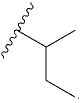 [SMILES: *C(C)CC]

In certain embodiments, R² is

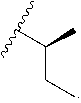 [SMILES: *[C@@H](C)CC]

In certain embodiments, R² is

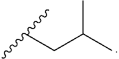 [SMILES: *CC(C)C]

In certain embodiments, R² is

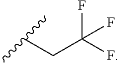 [SMILES: *CC(F)(F)F]

In certain embodiments, R² is

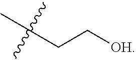 [SMILES: *CCO]

In certain embodiments, R² is

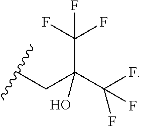 [SMILES: *CC(O)(C(F)(F)F)C(F)(F)F]

In certain embodiments, R² is

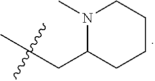 [SMILES: *CC1CCCCN1C]

In certain embodiments, R² is

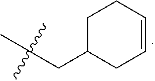 [SMILES: *CC1CC=CCC1]

In certain embodiments, R² is

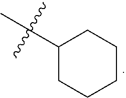 [SMILES: *C1CCCCC1]

In certain embodiments, R² is

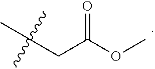 [SMILES: *CC(=O)OC]

In certain embodiments, R² is

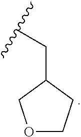 [SMILES: *CC1CCOC1]

In certain embodiments, R² is

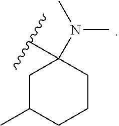

In certain embodiments, R² is

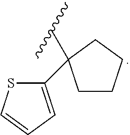 [SMILES: *C1(c2cccs2)CCCC1]

In certain embodiments, R² is

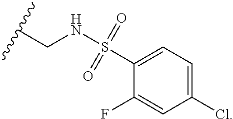 [SMILES: *CNS(=O)(=O)c1ccc(Cl)cc1F]

In certain embodiments, R² is

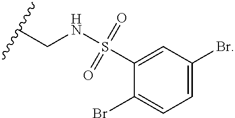 [SMILES: *CNS(=O)(=O)c1cc(Br)ccc1Br]

In certain embodiments, R² is

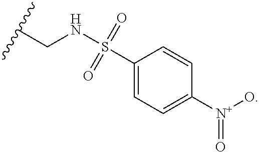

In certain embodiments, R² is

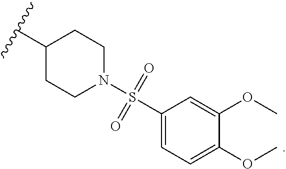 [SMILES: *C1CCN(S(=O)(=O)c2ccc(OC)c(OC)c2)CC1]

In certain embodiments, R² is

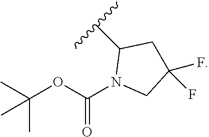 [SMILES: *C1CC(F)(F)CN1C(=O)OC(C)(C)C]

In certain embodiments, R² is

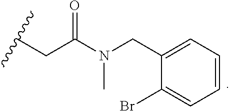 [SMILES: *CC(=O)N(C)Cc1ccccc1Br]

In certain embodiments, R² is

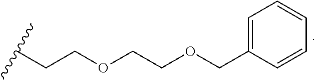 [SMILES: *CCOCCOCc1ccccc1]

In certain embodiments, R² is

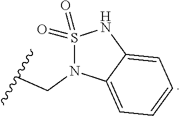 [SMILES: *CN1c2ccccc2NS1(=O)=O]

In certain embodiments, R² is

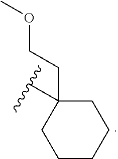 [SMILES: *C1(CCOC)CCCCC1]

In certain embodiments, R² is

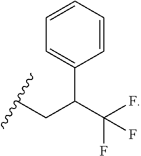 [SMILES: *CC(c1ccccc1)C(F)(F)F]

In certain embodiments, R² is

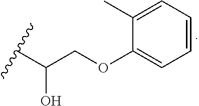 [SMILES: *C(O)COc1ccccc1C]

In certain embodiments, R² is

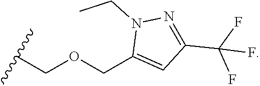 [SMILES: *COCc1cc(C(F)(F)F)nn1CC]

In certain embodiments, R² is

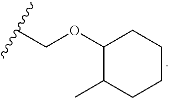 [SMILES: *COC1CCCCC1C]

In certain embodiments, R² is

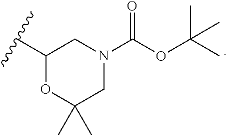 [SMILES: *C1CN(C(=O)OC(C)(C)C)CC(C)(C)O1]

In certain embodiments, R² is

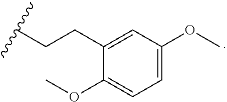 [SMILES: *CCc1cc(OC)ccc1OC]

In certain embodiments R² is

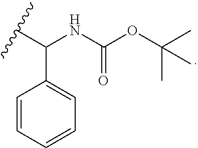 [SMILES: *C(NC(=O)OC(C)(C)C)c1ccccc1]

In certain embodiments, R² is

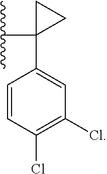 [SMILES: *C1(c2ccc(Cl)c(Cl)c2)CC1]

In certain embodiments, R² is

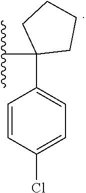 [SMILES: *C1(c2ccc(Cl)cc2)CCCC1]

In certain embodiments, R² is

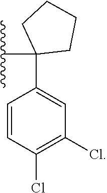 [SMILES: *C1(c2ccc(Cl)c(Cl)c2)CCCC1]

In certain embodiments, R² is

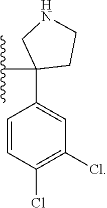 [SMILES: *C1(c2ccc(Cl)c(Cl)c2)CCNC1]

In certain embodiments, R² is

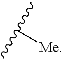

In certain embodiments, R³ is methyl. In certain embodiments, R³ is ethyl. In certain embodiments, R³ is n-propyl. In certain embodiments, R³ is i-propyl. In certain embodiments, R³ is t-butyl. In certain embodiments, R³ is

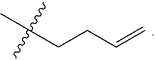 [SMILES: *CCC=C]

In certain embodiments, R³ is

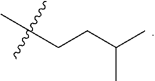 [SMILES: *CCC(C)C]

In certain embodiments, R³ is

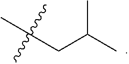 [SMILES: *CC(C)C]

In certain embodiments, R³ is

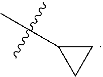 [SMILES: *C1CC1]

In certain embodiments, R³ is

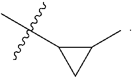 [SMILES: *C1CC1C]

In certain embodiments, R³ is

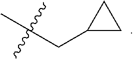 [SMILES: *CC1CC1]

In certain embodiments, R³ is

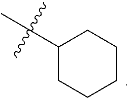 [SMILES: *C1CCCCC1]

In certain embodiments, R³ is

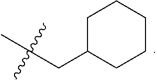 [SMILES: *CC1CCCCC1]

In certain embodiments, R³ is

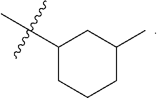 [SMILES: *C1CCCC(C)C1]

In certain embodiments, R³ is

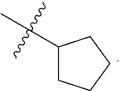 [SMILES: *C1CCCC1]

In certain embodiments, R³ is

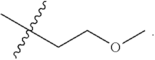 [SMILES: *CCOC]

In certain embodiments, R³ is

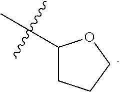 [SMILES: *C1CCCO1]

In certain embodiments, R³ is

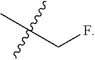 [SMILES: *CF]

In certain embodiments, R³ is

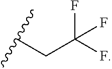 [SMILES: *CC(F)(F)F]

In certain embodiments, R³ is

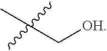 [SMILES: *CO]

In certain embodiments, R³ is

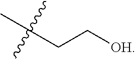 [SMILES: *CCO]

In certain embodiments, R³ is

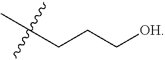 [SMILES: *CCCO]

In certain embodiments, R³ is

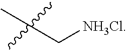

In certain embodiments, R³ is

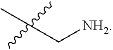 [SMILES: *CN]

In certain embodiments, R³ is

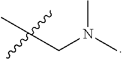 [SMILES: *CN(C)C]

In certain embodiments, R³ is

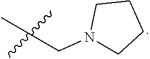 [SMILES: *CN1CCCC1]

In certain embodiments, R³ is

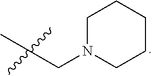 [SMILES: *CN1CCCCC1]

In certain embodiments, R³ is

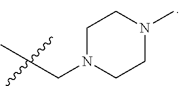 [SMILES: *CN1CCN(C)CC1]

In certain embodiments, R³ is

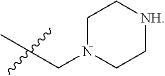 [SMILES: *CN1CCNCC1]

In certain embodiments, R³ is

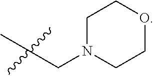 [SMILES: *CN1CCOCC1]

In certain embodiments, R³ is

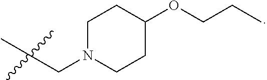 [SMILES: *CN1CCC(OCCC)CC1]

In certain embodiments, R³ is

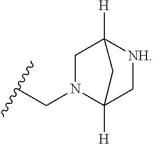 [SMILES: *CN1CC2CC1CN2]

In certain embodiments, R³ is

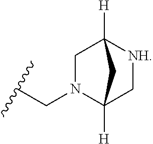 [SMILES: *CN1C[C@H]2C[C@@H]1CN2]

In certain embodiments, R³ is

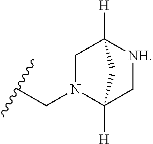 [SMILES: *CN1C[C@@H]2C[C@H]1CN2]

In certain embodiments, R³ is

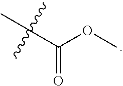 [SMILES: *C(=O)OC]

In certain embodiments, R³ is

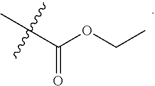 [SMILES: *C(=O)OCC]

In certain embodiments, R³ is

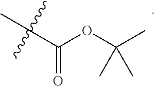 [SMILES: *C(=O)OC(C)(C)C]

In certain embodiments, R³ is

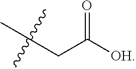 [SMILES: *CC(=O)O]

In certain embodiments, R³ is

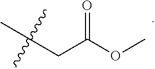 [SMILES: *CC(=O)OC]

In certain embodiments, R³ is

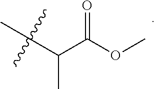 [SMILES: *C(C)C(=O)OC]

In certain embodiments, R³ is

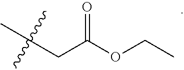 [SMILES: *CC(=O)OCC]

In certain embodiments, R³ is

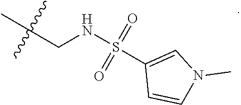 [SMILES: *CNS(=O)(=O)c1ccn(C)c1]

In certain embodiments, R³ is

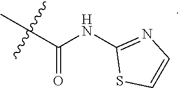 [SMILES: *C(=O)Nc1nccs1]

In certain embodiments, R³ is

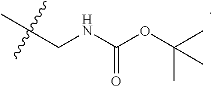 [SMILES: *CNC(=O)OC(C)(C)C]

In certain embodiments, R³ is

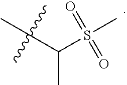 [SMILES: *C(C)S(C)(=O)=O]

In certain embodiments, R³ is

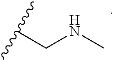 [SMILES: *CNC]

In certain embodiments, R³ is

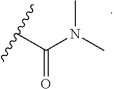 [SMILES: *C(=O)N(C)C]

In certain embodiments, R³ is

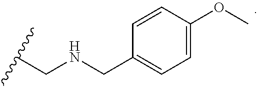 [SMILES: *CNCc1ccc(OC)cc1]

In certain embodiments, R⁴ is methyl. In certain embodiments, R⁴ is ethyl. In certain embodiments, R⁴ is n-propyl. In certain embodiments, R⁴ is

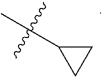 [SMILES: *C1CC1]

In certain embodiments, the compound of formula (II) is the compound of formula (IIa):

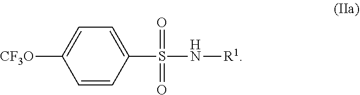

In certain embodiments, R¹ is

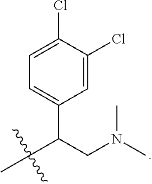 [SMILES: *C(CN(C)C)c1ccc(Cl)c(Cl)c1]

In certain embodiments, R¹ is

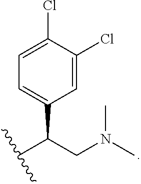 [SMILES: *[C@H](CN(C)C)c1ccc(Cl)c(Cl)c1]

In certain embodiments, R¹ is

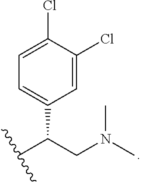 [SMILES: *[C@@H](CN(C)C)c1ccc(Cl)c(Cl)c1]

In certain embodiments, R¹ is

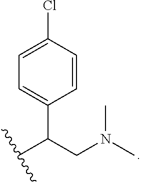 [SMILES: *C(CN(C)C)c1ccc(Cl)cc1]

In certain embodiments, R¹ is

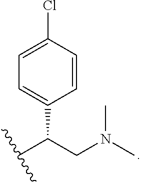 [SMILES: *[C@@H](CN(C)C)c1ccc(Cl)cc1]

In certain embodiments, R¹ is

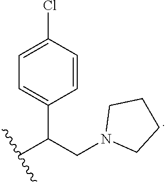 [SMILES: *C(CN1CCCC1)c1ccc(Cl)cc1]

In certain embodiments, R¹ is

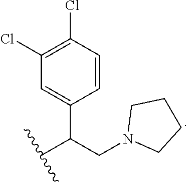 [SMILES: *C(CN1CCCC1)c1ccc(Cl)c(Cl)c1]

In certain embodiments, R¹ is

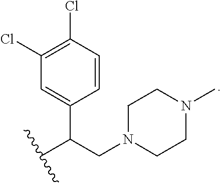 [SMILES: *C(CN1CCN(C)CC1)c1ccc(Cl)c(Cl)c1]

In certain embodiments, R¹ is

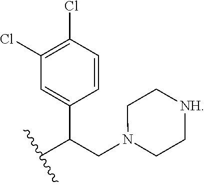 [SMILES: *C(CN1CCNCC1)c1ccc(Cl)c(Cl)c1]

In certain embodiments, R¹ is

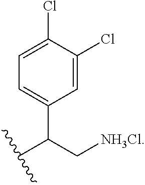

In certain embodiments, R¹ is

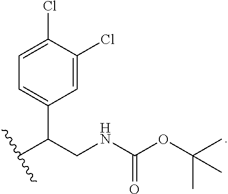 [SMILES: *C(CNC(=O)OC(C)(C)C)c1ccc(Cl)c(Cl)c1]

In certain embodiments, R¹ is

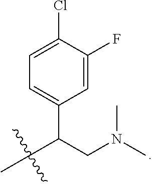 [SMILES: *C(CN(C)C)c1ccc(Cl)c(F)c1]

In certain embodiments, R¹ is

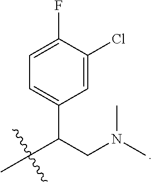 [SMILES: *C(CN(C)C)c1ccc(F)c(Cl)c1]

In certain embodiments, R¹ is

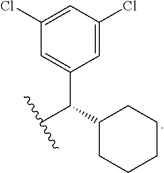 [SMILES: *[C@H](c1cc(Cl)cc(Cl)c1)C1CCCCC1]

In certain embodiments, R¹ is

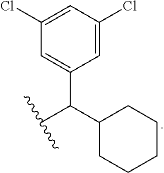 [SMILES: *C(c1cc(Cl)cc(Cl)c1)C1CCCCC1]

In certain embodiments, R¹ is

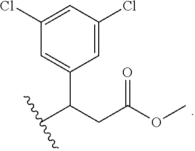 [SMILES: *C(CC(=O)OC)c1cc(Cl)cc(Cl)c1]

In certain embodiments, R¹ is

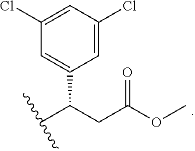 [SMILES: *[C@@H](CC(=O)OC)c1cc(Cl)cc(Cl)c1]

In certain embodiments, R¹ is

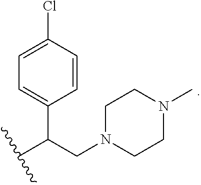 [SMILES: *C(CN1CCN(C)CC1)c1ccc(Cl)cc1]

In certain embodiments, R¹ is

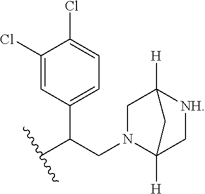 [SMILES: *C(CN1CC2CC1CN2)c1ccc(Cl)c(Cl)c1]

In certain embodiments, R¹ is

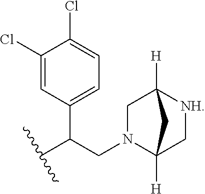 [SMILES: *C(CN1C[C@H]2C[C@@H]1CN2)c1ccc(Cl)c(Cl)c1]

In certain embodiments, R¹ is

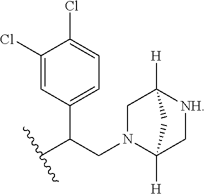 [SMILES: *C(CN1C[C@@H]2C[C@H]1CN2)c1ccc(Cl)c(Cl)c1]

In certain embodiments, R¹ is

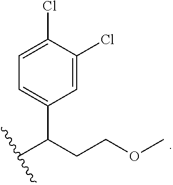 [SMILES: *C(CCOC)c1ccc(Cl)c(Cl)c1]

In certain embodiments, R¹ is

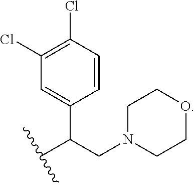 [SMILES: *C(CN1CCOCC1)c1ccc(Cl)c(Cl)c1]

In certain embodiments, R¹ is

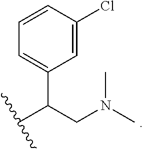 [SMILES: *C(CN(C)C)c1cccc(Cl)c1]

In certain embodiments, R¹ is

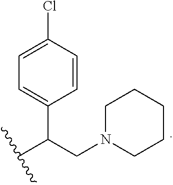 [SMILES: *C(CN1CCCCC1)c1ccc(Cl)cc1]

In certain embodiments, R¹ is

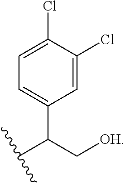 [SMILES: *C(CO)c1ccc(Cl)c(Cl)c1]

In certain embodiments, R¹ is

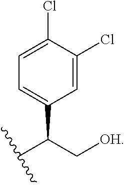 [SMILES: *[C@H](CO)c1ccc(Cl)c(Cl)c1]

In certain embodiments, R¹ is

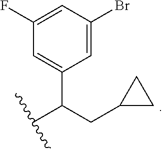 [SMILES: *C(CC1CC1)c1cc(F)cc(Br)c1]

In certain embodiments, R¹ is

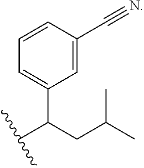 [SMILES: *C(CC(C)C)c1cccc(C#N)c1]

In certain embodiments, R¹ is

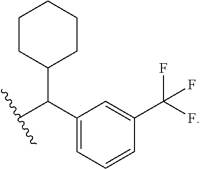 [SMILES: *C(c1cccc(C(F)(F)F)c1)C1CCCCC1]

In certain embodiments, R¹ is

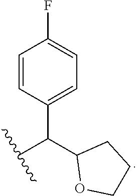 [SMILES: *C(c1ccc(F)cc1)C1CCCO1]

In certain embodiments, R¹ is

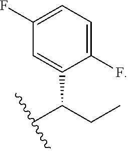 [SMILES: *[C@@H](CC)c1cc(F)ccc1F]

In certain embodiments, R¹ is

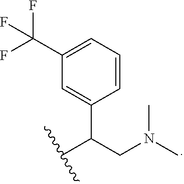 [SMILES: *C(CN(C)C)c1cccc(C(F)(F)F)c1]

In certain embodiments, R¹ is

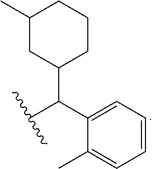 [SMILES: *C(c1ccccc1C)C1CCCC(C)C1]

In certain embodiments, R¹ is

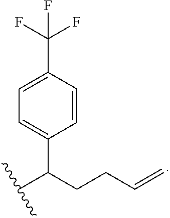 [SMILES: *C(CCC=C)c1ccc(C(F)(F)F)cc1]

In certain embodiments, R¹ is

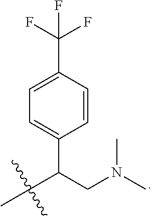 [SMILES: *C(CN(C)C)c1ccc(C(F)(F)F)cc1]

In certain embodiments, R¹ is

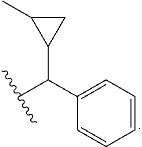 [SMILES: *C(c1ccccc1)C1CC1C]

In certain embodiments, R¹ is

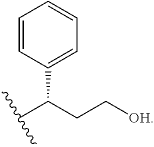 [SMILES: *[C@@H](CCO)c1ccccc1]

In certain embodiments, R¹ is

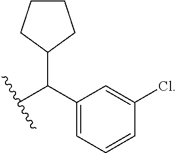 [SMILES: *C(c1cccc(Cl)c1)C1CCCC1]

In certain embodiments, R¹ is

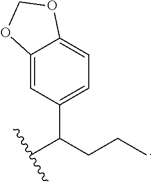 [SMILES: *C(CCC)c1ccc2c(c1)OCO2]

In certain embodiments, R¹ is

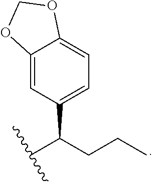 [SMILES: *[C@H](CCC)c1ccc2c(c1)OCO2]

In certain embodiments, R¹ is

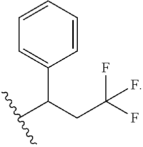 [SMILES: *C(CC(F)(F)F)c1ccccc1]

In certain embodiments, R¹ is

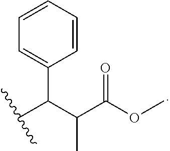 [SMILES: *C(c1ccccc1)C(C)C(=O)OC]

In embodiments, R¹ is

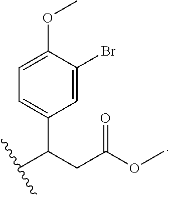 [SMILES: *C(CC(=O)OC)c1ccc(OC)c(Br)c1]

In certain embodiments, R¹ is

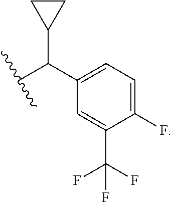 [SMILES: *C(c1ccc(F)c(C(F)(F)F)c1)C1CC1]

In certain embodiments, R¹ is

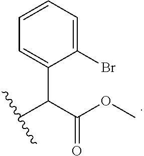 [SMILES: *C(C(=O)OC)c1ccccc1Br]

In certain embodiments, R¹ is

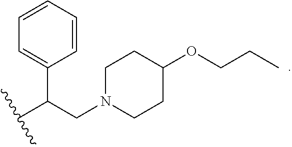 [SMILES: *C(CN1CCC(OCCC)CC1)c1ccccc1]

In certain embodiments, R¹ is

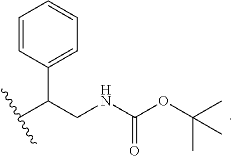 [SMILES: *C(CNC(=O)OC(C)(C)C)c1ccccc1]

In certain embodiments, R¹ is

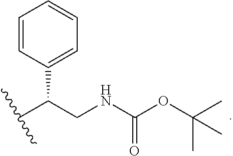 [SMILES: *[C@@H](CNC(=O)OC(C)(C)C)c1ccccc1]

In certain embodiments, R¹ is

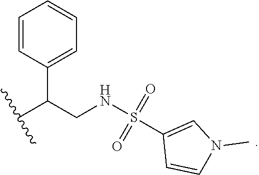 [SMILES: *C(CNS(=O)(=O)c1ccn(C)c1)c1ccccc1]

In certain embodiments, R¹ is

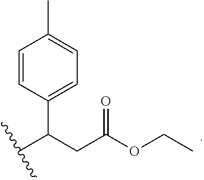 [SMILES: *C(CC(=O)OCC)c1ccc(C)cc1]

In certain embodiments, R¹ is

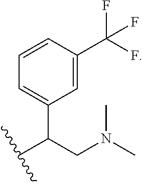 [SMILES: *C(CN(C)C)c1cccc(C(F)(F)F)c1]

In certain embodiments, R¹ is

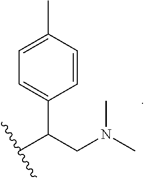 [SMILES: *C(CN(C)C)c1ccc(C)cc1]

In certain embodiments, R¹ is

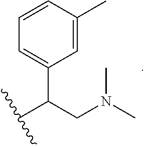 [SMILES: *C(CN(C)C)c1cccc(C)c1]

In certain embodiments, R¹ is

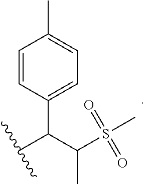 [SMILES: *C(c1ccc(C)cc1)C(C)S(C)(=O)=O]

In certain embodiments, R¹ is

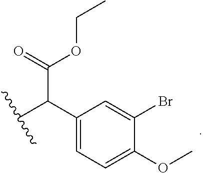 [SMILES: *C(C(=O)OCC)c1ccc(OC)c(Br)c1]

In certain embodiments, R¹ is

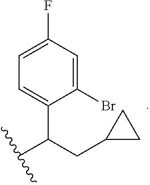 [SMILES: *C(CC1CC1)c1ccc(F)cc1Br]

In certain embodiments, R¹ is

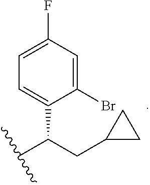 [SMILES: *[C@@H](CC1CC1)c1ccc(F)cc1Br]

In certain embodiments, R¹ is

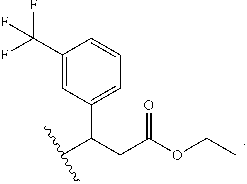 [SMILES: *C(CC(=O)OCC)c1cccc(C(F)(F)F)c1]

In certain embodiments, R¹ is

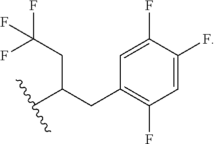 [SMILES: *C(Cc1cc(F)c(F)cc1F)CC(F)(F)F]

In certain embodiments, R¹ is

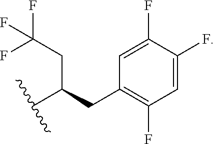 [SMILES: *[C@H](Cc1cc(F)c(F)cc1F)CC(F)(F)F]

In certain embodiments, R¹ is

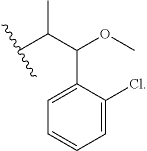 [SMILES: *C(C)C(OC)c1ccccc1Cl]

In certain embodiments, R¹ is

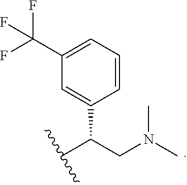 [SMILES: *[C@@H](CN(C)C)c1cccc(C(F)(F)F)c1]

In certain embodiments, R¹ is

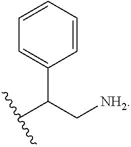 [SMILES: *C(CN)c1ccccc1]

In certain embodiments, R¹ is

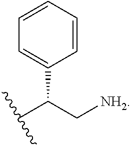 [SMILES: *[C@@H](CN)c1ccccc1]

In certain embodiments, R¹ is

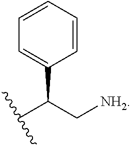 [SMILES: *[C@H](CN)c1ccccc1]

In certain embodiments, R¹ is

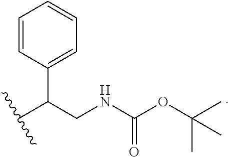 [SMILES: *C(CNC(=O)OC(C)(C)C)c1ccccc1]

In certain embodiments, R¹ is

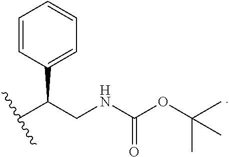 [SMILES: *[C@H](CNC(=O)OC(C)(C)C)c1ccccc1]

In certain embodiments, R¹ is

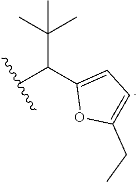 [SMILES: *C(c1ccc(CC)o1)C(C)(C)C]

In certain embodiments, R¹ is

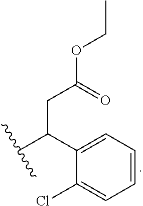 [SMILES: *C(CC(=O)OCC)c1ccccc1Cl]

In certain embodiments, R¹ is

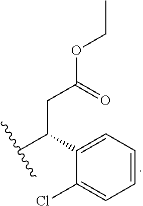 [SMILES: *[C@H](CC(=O)OCC)c1ccccc1Cl]

In certain embodiments, R¹ is

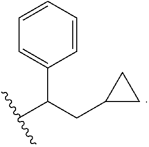 [SMILES: *C(CC1CC1)c1ccccc1]

In certain embodiments, R¹ is

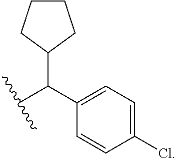 [SMILES: *C(c1ccc(Cl)cc1)C1CCCC1]

In certain embodiments, R¹ is

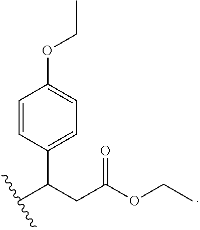 [SMILES: *C(CC(=O)OCC)c1ccc(OCC)cc1]

In certain embodiments, R¹ is

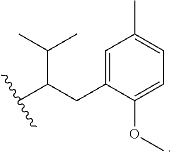 [SMILES: *C(Cc1cc(C)ccc1OC)C(C)C]

In certain embodiments, R¹ is

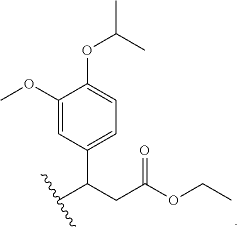 [SMILES: *C(CC(=O)OCC)c1ccc(OC(C)C)c(OC)c1]

In certain embodiments, R¹ is

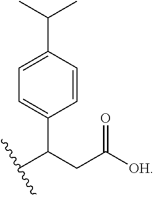 [SMILES: *C(CC(=O)O)c1ccc(C(C)C)cc1]

In certain embodiments, R¹ is

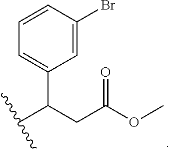 [SMILES: *C(CC(=O)OC)c1cccc(Br)c1]

In certain embodiments, R¹ is

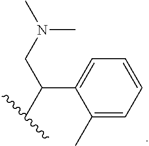 [SMILES: *C(CN(C)C)c1ccccc1C]

In certain embodiments, R¹ is

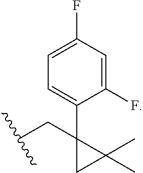 [SMILES: *CC1(c2ccc(F)cc2F)CC1(C)C]

In certain embodiments, R¹ is

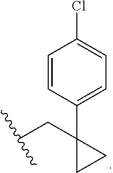 [SMILES: *CC1(c2ccc(Cl)cc2)CC1]

In certain embodiments, R¹ is

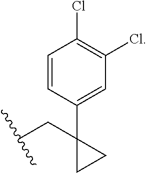 [SMILES: *CC1(c2ccc(Cl)c(Cl)c2)CC1]

In certain embodiments, R¹ is

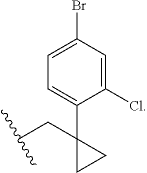 [SMILES: *CC1(c2ccc(Br)cc2Cl)CC1]

In certain embodiments, R¹ is

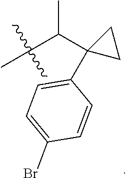 [SMILES: *C(C)C1(c2ccc(Br)cc2)CC1]

In certain embodiments, R¹ is

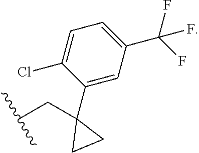 [SMILES: *CC1(c2cc(C(F)(F)F)ccc2Cl)CC1]

In certain embodiments, R¹ is

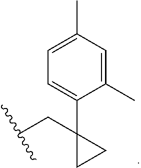 [SMILES: *CC1(c2ccc(C)cc2C)CC1]

In certain embodiments, R¹ is

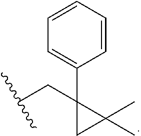 [SMILES: *CC1(c2ccccc2)CC1(C)C]

In certain embodiments, R¹ is

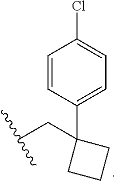 [SMILES: *CC1(c2ccc(Cl)cc2)CCC1]

In certain embodiments, R¹ is

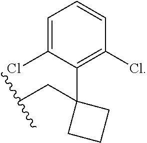 [SMILES: *CC1(c2c(Cl)cccc2Cl)CCC1]

In certain embodiments, R¹ is

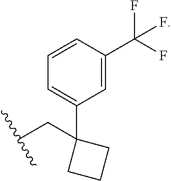 [SMILES: *CC1(c2cccc(C(F)(F)F)c2)CCC1]

In certain embodiments, R¹ is

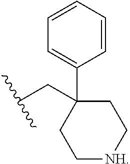 [SMILES: *CC1(c2ccccc2)CCNCC1]

In certain embodiments, R¹ is

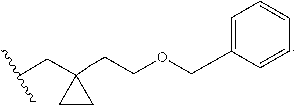 [SMILES: *CC1(CCCOCCc2ccccc2)CC1]

In certain embodiments, R¹ is

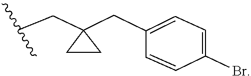 [SMILES: *CC1(Cc2ccc(Br)cc2)CC1]

In certain embodiments, R¹ is

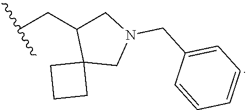 [SMILES: *CC1CN(Cc2ccccc2)CC12CCC2]

In certain embodiments, R¹ is

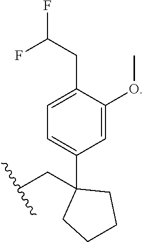 [SMILES: *CC1(c2ccc(CC(F)F)c(OC)c2)CCCC1]

In certain embodiments, R¹ is

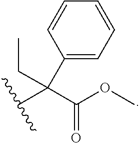 [SMILES: *C(CC)(C(=O)OC)c1ccccc1]

In certain embodiments, R¹ is

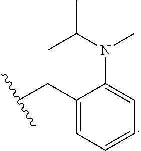 [SMILES: *Cc1ccccc1N(C)C(C)C]

In certain embodiments, R¹ is

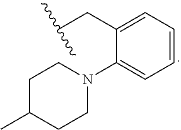 [SMILES: *Cc1ccccc1N1CCC(C)CC1]

In certain embodiments, R¹ is

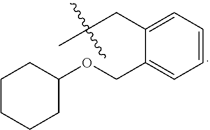 [SMILES: *Cc1ccccc1COC1CCCCC1]

In certain embodiments, R¹ is

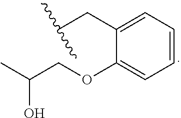 [SMILES: *Cc1ccccc1OCC(C)O]

In certain embodiments, R¹ is

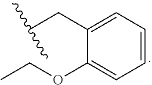 [SMILES: *Cc1ccccc1OCC]

In certain embodiments, R¹ is

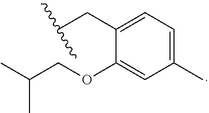 [SMILES: *Cc1ccc(C)cc1OCC(C)C]

In certain embodiments, R¹ is

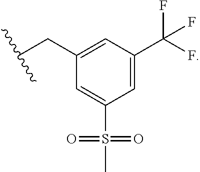 [SMILES: *Cc1cc(C(F)(F)F)cc(S(C)(=O)=O)c1]

In certain embodiments, R¹ is

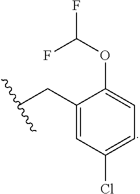 [SMILES: *Cc1cc(Cl)ccc1OC(F)F]

In certain embodiments, R¹ is

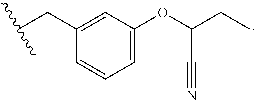 [SMILES: *Cc1cccc(OC(C#N)CC)c1]

In certain embodiments, R¹ is

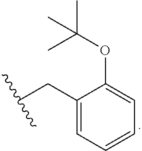 [SMILES: *Cc1ccccc1OC(C)(C)C]

In certain embodiments, R¹ is

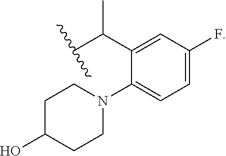 [SMILES: *C(C)c1cc(F)ccc1N1CCC(O)CC1]

In certain embodiments, R¹ is

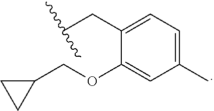 [SMILES: *Cc1ccc(C)cc1OCC1CC1]

In certain embodiments, R¹ is

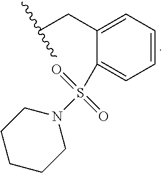 [SMILES: *Cc1ccccc1S(=O)(=O)N1CCCCC1]

In certain embodiments, R¹ is

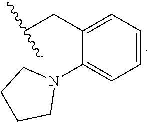 [SMILES: *Cc1ccccc1N1CCCC1]

In certain embodiments, R¹ is

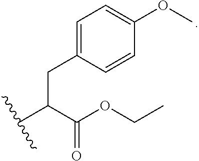 [SMILES: *C(Cc1ccc(OC)cc1)C(=O)OCC]

In certain embodiments, R¹ is

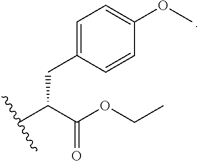 [SMILES: *[C@H](Cc1ccc(OC)cc1)C(=O)OCC]

In certain embodiments, R¹ is

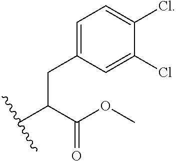 [SMILES: *C(Cc1ccc(Cl)c(Cl)c1)C(=O)OC]

In certain embodiments, R¹ is

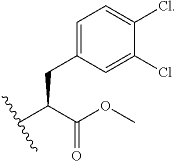 [SMILES: *[C@@H](Cc1ccc(Cl)c(Cl)c1)C(=O)OC]

In certain embodiments, R¹ is

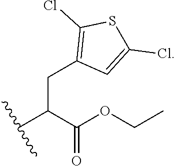 [SMILES: *C(Cc1cc(Cl)sc1Cl)C(=O)OCC]

In certain embodiments, R¹ is

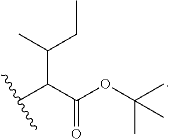 [SMILES: *C(C(=O)OC(C)(C)C)C(C)CC]

In certain embodiments, R¹ is

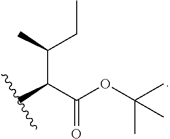 [SMILES: *[C@H](C(=O)OC(C)(C)C)[C@@H](C)CC]

In certain embodiments, R¹ is

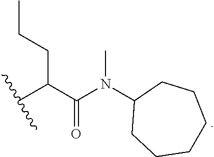 [SMILES: *C(CCC)C(=O)N(C)C1CCCCCC1]

In certain embodiments, R¹ is

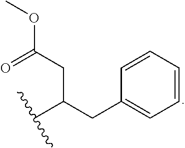 [SMILES: *C(CC(=O)OC)Cc1ccccc1]

In certain embodiments, R¹ is

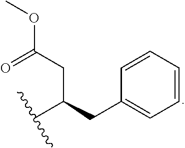 [SMILES: *[C@@H](CC(=O)OC)Cc1ccccc1]

In certain embodiments, R¹ is

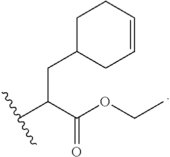 [SMILES: *C(CC1CC=CCC1)C(=O)OCC]

In certain embodiments, R¹ is

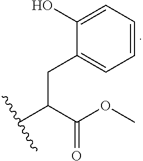 [SMILES: *C(Cc1ccccc1O)C(=O)OC]

In certain embodiments, R¹ is

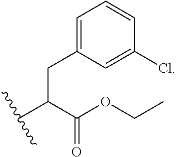 [SMILES: *C(Cc1cccc(Cl)c1)C(=O)OCC]

In certain embodiments, R¹ is

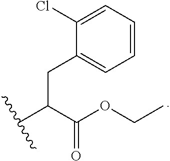 [SMILES: *C(Cc1ccccc1Cl)C(=O)OCC]

In certain embodiments, R¹ is

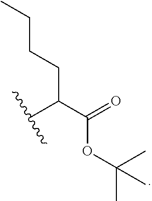 [SMILES: *C(CCCC)C(=O)OC(C)(C)C]

In certain embodiments, R¹ is

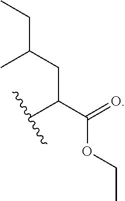 [SMILES: *C(CC(C)CC)C(=O)OCC]

In certain embodiments, R¹ is

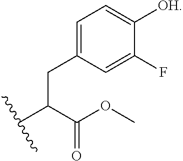 [SMILES: *C(Cc1ccc(O)c(F)c1)C(=O)OC]

In certain embodiments, R¹ is

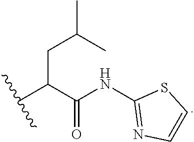 [SMILES: *C(CC(C)C)C(=O)Nc1nccs1]

In certain embodiments, R¹ is

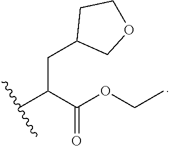 [SMILES: *C(CC1CCOC1)C(=O)OCC]

In certain embodiments, R¹ is

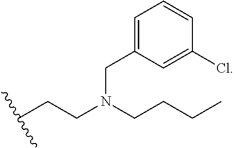 [SMILES: *CCN(CCCC)Cc1cccc(Cl)c1]

In certain embodiments, R¹ is

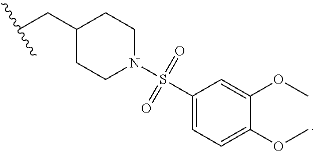 [SMILES: *CC1CCN(S(=O)(=O)c2ccc(OC)c(OC)c2)CC1]

In certain embodiments, R¹ is

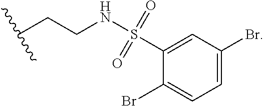 [SMILES: *CCNS(=O)(=O)c1cc(Br)ccc1Br]

In certain embodiments, R¹ is

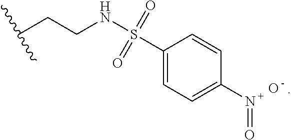

In certain embodiments, R¹ is

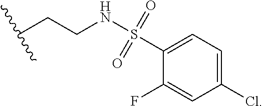 [SMILES: *CCNS(=O)(=O)c1ccc(Cl)cc1F]

In certain embodiments, R¹ is

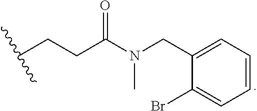 [SMILES: *CCC(=O)N(C)Cc1ccccc1Br]

In certain embodiments, R¹ is

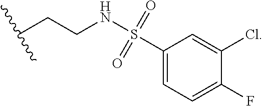 [SMILES: *CCNS(=O)(=O)c1ccc(F)c(Cl)c1]

In certain embodiments, R¹ is

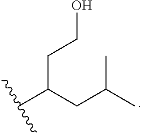 [SMILES: *C(CCO)CC(C)C]

In certain embodiments, R¹ is

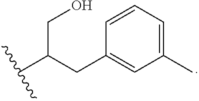 [SMILES: *C(CO)Cc1cccc(C)c1]

In certain embodiments, R¹ is

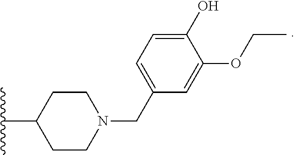 [SMILES: *C1CCN(Cc2ccc(O)c(OCC)c2)CC1]

In certain embodiments, R¹ is

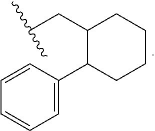 [SMILES: *CC1CCCCC1c1ccccc1]

In certain embodiments, R¹ is

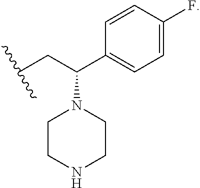 [SMILES: *C[C@H](c1ccc(F)cc1)N1CCNCC1]

In certain embodiments, R¹ is

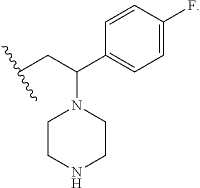 [SMILES: *CC(c1ccc(F)cc1)N1CCNCC1]

In certain embodiments, R¹ is

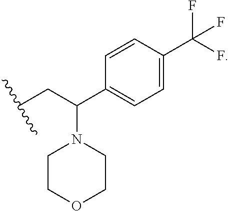 [SMILES: *CC(c1ccc(C(F)(F)F)cc1)N1CCOCC1]

In certain embodiments, R¹ is

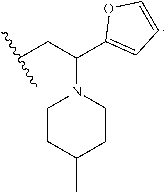 [SMILES: *CC(c1ccco1)N1CCC(C)CC1]

In certain embodiments, R¹ is

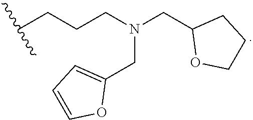 [SMILES: *CCCN(Cc1ccco1)CC1CCCO1]

In certain embodiments, R¹ is

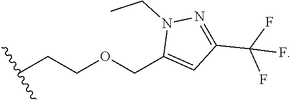 [SMILES: *CCOCc1cc(C(F)(F)F)nn1CC]

In certain embodiments, R¹ is

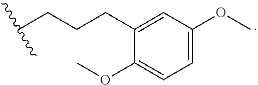 [SMILES: *CCCc1cc(OC)ccc1OC]

In certain embodiments, R¹ is

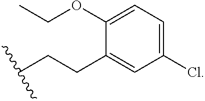 [SMILES: *CCc1cc(Cl)ccc1OCC]

In certain embodiments, R¹ is

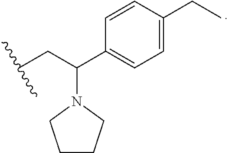 [SMILES: *CC(c1ccc(CC)cc1)N1CCCC1]

In certain embodiments, R¹ is

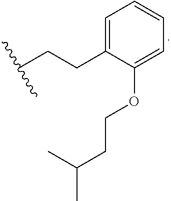 [SMILES: *CCc1ccccc1OCCC(C)C]

In certain embodiments, R¹ is

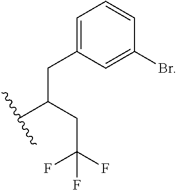 [SMILES: *C(Cc1cccc(Br)c1)CC(F)(F)F]

In certain embodiments, R¹ is

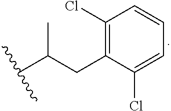 [SMILES: *C(C)Cc1c(Cl)cccc1Cl]

In certain embodiments, R¹ is

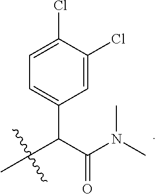 [SMILES: *C(C(=O)N(C)C)c1ccc(Cl)c(Cl)c1]

In certain embodiments, R¹ is

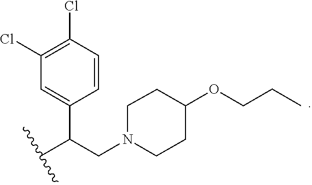 [SMILES: *C(CN1CCC(OCCC)CC1)c1ccc(Cl)c(Cl)c1]

In certain embodiments, R¹ is

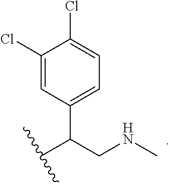 [SMILES: *C(CNC)c1ccc(Cl)c(Cl)c1]

In certain embodiments, R¹ is

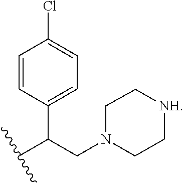 [SMILES: *C(CN1CCNCC1)c1ccc(Cl)cc1]

In certain embodiments, R¹ is

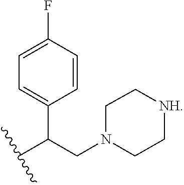 [SMILES: *C(CN1CCNCC1)c1ccc(F)cc1]

In certain embodiments, R¹ is

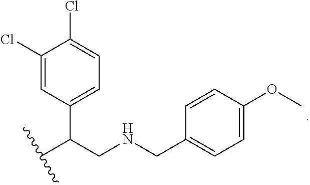 [SMILES: *C(CNCc1ccc(OC)cc1)c1ccc(Cl)c(Cl)c1]

In certain embodiments, R¹ is

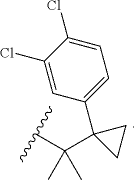 [SMILES: *C(C)(C)C1(c2ccc(Cl)c(Cl)c2)CC1]

In certain embodiments, R¹ is

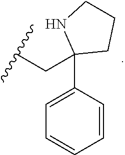 [SMILES: *CC1(c2ccccc2)CCCN1]

In certain embodiments, R¹ is

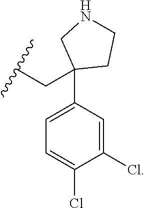 [SMILES: *CC1(c2ccc(Cl)c(Cl)c2)CCNC1]

In certain embodiments, R¹ is

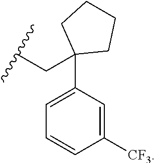 [SMILES: *CC1(c2cccc(C(F)(F)F)c2)CCCC1]

In certain embodiments, R¹ is

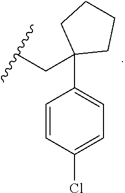 [SMILES: *CC1(c2ccc(Cl)cc2)CCCC1]

In certain embodiments, R¹ is

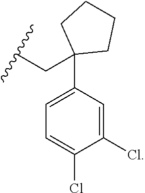 [SMILES: *CC1(c2ccc(Cl)c(Cl)c2)CCCC1]

In certain embodiments, R¹ is

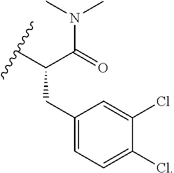 [SMILES: *[C@@H](Cc1ccc(Cl)c(Cl)c1)C(=O)N(C)C]

In certain embodiments, R¹ is

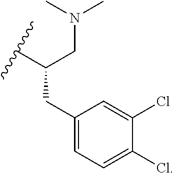 [SMILES: *[C@@H](Cc1ccc(Cl)c(Cl)c1)CN(C)C]

In certain embodiments, R¹ is

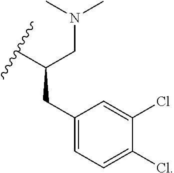 [SMILES: *[C@H](Cc1ccc(Cl)c(Cl)c1)CN(C)C]

In certain embodiments, the compound of formula (II) is the compound of formula (IIb):

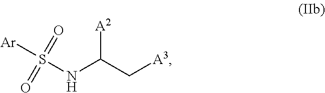

wherein:
A² is selected from the group consisting of

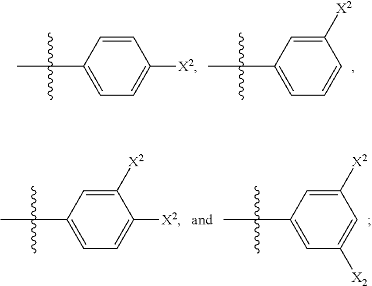

each occurrence of X² is independently selected from the group consisting of F, Cl, Br, and CF₃;
A³ is selected from the group consisting of NMe₂,

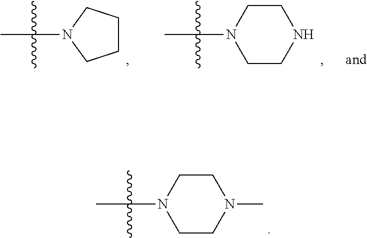

In certain embodiments, A² is

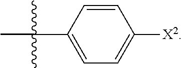

In certain embodiments, A² is

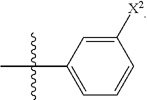

In certain embodiments, A² is

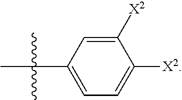

In certain embodiments, A² is

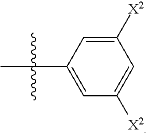

In certain embodiments, A² is

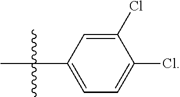 [SMILES: *c1ccc(Cl)c(Cl)c1]

In certain embodiments, X² is F. In certain embodiments, X² is Cl. In certain embodiments, X² is Br. In certain embodiments, X² is CF₃.

In certain embodiments, A³ is NMe₂. In certain embodiments, A³ is NH₃Cl. In certain embodiments, A³ is NHMe. In certain embodiments, A³ is NHC(═O)Ot-Bu. In certain embodiments, A³ is

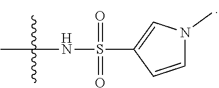 [SMILES: *NS(=O)(=O)c1ccn(C)c1]

In certain embodiments, A³ is

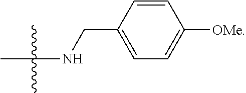 [SMILES: *NCc1ccc(OC)cc1]

In certain embodiments, A³ is

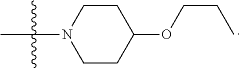 [SMILES: *N1CCC(OCCC)CC1]

In certain embodiments, A³ is

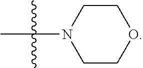 [SMILES: *N1CCOCC1]

In certain embodiments, A³ is

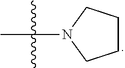 [SMILES: *N1CCCC1]

In certain embodiments A³ is

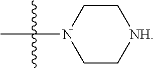 [SMILES: *N1CCNCC1]

In certain embodiments, A³ is

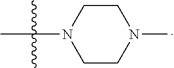 [SMILES: *N1CCN(C)CC1]

In certain embodiments, A³ is

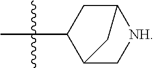 [SMILES: *C1CC2CC1CN2]

In certain embodiments, A³ is

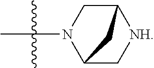 [SMILES: *N1C[C@H]2C[C@@H]1CN2]

In certain embodiments, A³ is

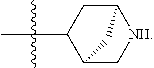 [SMILES: *C1C[C@@H]2C[C@H]1CN2]

In certain embodiments, the compound of formula (I) is selected from the group consisting of
N-(1-(3,4-dichlorophenyl)-2-(dimethylamino)ethyl)-3-nitro-4-(trifluoromethoxy)benzenesulfonamide;
N-(1-hydroxy-5-methylhexan-3-yl)-4-(trifluoromethoxy)benzenesulfonamide;
N-(1-(3,4-dichlorophenyl)-2-(dimethylamino)ethyl)-4-pentylbenzenesulfonamide;
N-(cyclohexyl(3,5-dichlorophenyl)methyl)-4-(trifluoromethoxy)benzenesulfonamide;
N-(1-(3,4-dichlorophenyl)-2-(dimethylamino)ethyl)-4-(4-(trifluoromethyl)phenoxy)benzenesulfonamide;
N-(1-(3,4-dichlorophenyl)-2-(dimethylamino)ethyl)-4-phenoxybenzenesulfonamide;
N-(1-(3,4-dichlorophenyl)-2-(dimethylamino)ethyl)-[1,1′-biphenyl]-4-sulfonamide;
N-(1-(4-chlorophenyl)-2-(dimethylamino)ethyl)-4-phenoxybenzenesulfonamide;
N-(2-(dimethylamino)-1-(3-(trifluoromethyl)phenyl)ethyl)-4-phenoxybenzenesulfonamide;
3-chloro-N-(1-(3,4-dichlorophenyl)-2-(dimethylamino)ethyl)-4-(trifluoromethoxy)benzenesulfonamide;
4-(cyclopentyloxy)-N-(1-(3,4-dichlorophenyl)-2-(dimethylamino)ethyl)benzenesulfonamide;
N-(1-(3,4-dichlorophenyl)-2-(dimethylamino)ethyl)-4-propylbenzenesulfonamide;
N-(1-(3,4-dichlorophenyl)-2-(4-methylpiperazin-1-yl)ethyl)-4-(trifluoromethoxy)benzenesulfonamide;
N-(1-(3,4-dichlorophenyl)-2-(dimethylamino)ethyl)-[1,1′-biphenyl]-3-sulfonamide;
3-chloro-N-(1-(4-chlorophenyl)-2-(dimethylamino)ethyl)-4-(trifluoromethoxy)benzenesulfonamide;
3-chloro-N-(2-(dimethylamino)-1-(3-(trifluoromethyl)phenyl)ethyl)-4-(trifluoromethoxy)benzenesulfonamide;
N-(1-(3-bromo-5-fluorophenyl)-2-cyclopropylethyl)-4-(trifluoromethoxy)benzenesulfonamide;
N-((1-(2,4-difluorophenyl)-2,2-dimethylcyclopropyl)methyl)-4-(trifluoromethoxy)benzenesulfonamide;
N-(1-(3,4-dichlorophenyl)-2-(dimethylamino)ethyl)-4-(trifluoromethoxy)benzenesulfonamide;
N-(2-(dimethylamino)-1-(3-(trifluoromethyl)phenyl)ethyl)-4-(4-(trifluoromethyl)phenoxy)benzenesulfonamide;
N-(2-(isopropyl(methyl)amino)benzyl)-4-(trifluoromethoxy)benzenesulfonamide;
4-(trifluoromethoxy)-N-(1-(4-(trifluoromethyl)phenyl)pent-4-en-1-yl)benzenesulfonamide;
N-(1-(3,4-dichlorophenyl)-2-(dimethylamino)ethyl)-3-(trifluoromethoxy)benzenesulfonamide;
2-amino-N-(1-(3,4-dichlorophenyl)-2-(dimethylamino)ethyl)-4-(trifluoromethoxy)benzenesulfonamide;
N-(1-(3,4-dichlorophenyl)-2-(dimethylamino)ethyl)naphthalene-1-sulfonamide;
N-(2-((cyclohexyloxy)methyl)benzyl)-4-(trifluoromethoxy)benzenesulfonamide;
N-((1-(4-bromo-2-chlorophenyl)cyclopropyl)methyl)-4-(trifluoromethoxy)benzenesulfonamide;
N-(2-(4-methylpiperidin-1-yl)benzyl)-4-(trifluoromethoxy)benzenesulfonamide;
N-(1-(4-chlorophenyl)-2-(4-methylpiperazin-1-yl)ethyl)-4-(trifluoromethoxy)benzenesulfonamide;
2-chloro-N-(1-(3,4-dichlorophenyl)-2-(dimethylamino)ethyl)-4-(trifluoromethoxy)benzenesulfonamide;
N-(1-(3,4-dichlorophenyl)-2-(dimethylamino)ethyl)naphthalene-2-sulfonamide;
3,4-dichloro-N-(1-(3,4-dichlorophenyl)-2-(dimethylamino)ethyl)benzenesulfonamide;
N-((2-(4-methoxyphenyl)-1-methylpyrrolidin-3-yl)methyl)-4-(trifluoromethoxy)benzenesulfonamide;
N-(1-hydroxy-3-(m-tolyl)propan-2-yl)-4-(trifluoromethoxy)benzenesulfonamide;
N-(1-(3,4-dichlorophenyl)-2-(dimethylamino)ethyl)-2-phenylethane-1-sulfonamide;
N-(1-(3,4-dichlorophenyl)-2-(pyrrolidin-1-yl)ethyl)-4-(trifluoromethoxy)benzenesulfonamide;
2,5-dibromo-N-(2-((4-(trifluoromethoxy)phenyl)sulfonamido)ethyl)benzenesulfonamide;
N-(1-(3,4-dichlorophenyl)-2-(dimethylamino)ethyl)-N′-methyl-4-(trifluoromethoxy)benzenesulfonimidamide;
N-(1-(3,4-dichlorophenyl)-2-(dimethylamino)ethyl)-2,2-difluorobenzo[d][1,3]dioxole-5-sulfonamide;
ethyl 3-((4-(trifluoromethoxy)phenyl)sulfonamido)-3-(3-(trifluoromethyl)phenyl)propanoate;
N-(1-(1-(4-bromophenyl)cyclopropyl)ethyl)-4-(trifluoromethoxy)benzenesulfonamide;
N-((1-(2-chloro-5-(trifluoromethyl)phenyl)cyclopropyl)methyl)-4-(trifluoromethoxy)benzenesulfonamide;
4-methyl-N-(thiazol-2-yl)-2-((4-(trifluoromethoxy)phenyl)sulfonamido)pentanamide;
N-(1-(3,4-dichlorophenyl)-2-(dimethylamino)ethyl)-4-isopropylbenzenesulfonamide;
N-(1-(3,4-dichlorophenyl)-2-(dimethylamino)ethyl)-4-(trifluoromethyl)benzenesulfonamide;
N-(4,4,4-trifluoro-1-(2,4,5-trifluorophenyl)butan-2-yl)-4-(trifluoromethoxy)benzenesulfonamide;
N-((6-benzyl-6-azaspiro[3.4]octan-8-yl)methyl)-4-(trifluoromethoxy)benzenesulfonamide;
N-(2-((1-ethyl-3-(trifluoromethyl)-1H-pyrazol-5-yl)methoxy)ethyl)-4-(trifluoromethoxy)benzenesulfonamide;
4-chloro-N-(1-(3,4-dichlorophenyl)-2-(dimethylamino)ethyl)benzenesulfonamide;
N-(1-(3-cyanophenyl)-3-methylbutyl)-4-(trifluoromethoxy)benzenesulfonamide;
N-(cyclohexyl(3-(trifluoromethyl)phenyl)methyl)-4-(trifluoromethoxy)benzenesulfonamide;
N-((1-(2,6-dichlorophenyl)cyclobutyl)methyl)-4-(trifluoromethoxy)benzenesulfonamide;
N-(2-(cyclopropylmethoxy)-4-methylbenzyl)-4-(trifluoromethoxy)benzenesulfonamide;
(E)-N-(1-(3,4-dichlorophenyl)-2-(dimethylamino)ethyl)-2-phenylethene-1-sulfonamide;
N-(2-(2-hydroxypropoxy)benzyl)-4-(trifluoromethoxy)benzenesulfonamide;
3-chloro-4-fluoro-N-(2-((4-(trifluoromethoxy)phenyl)sulfonamido)ethyl)benzenesulfonamide;
3-amino-N-(1-(3,4-dichlorophenyl)-2-(dimethylamino)ethyl)-4-(trifluoromethoxy)benzenesulfonamide;
N-(2-(butyl(3-chlorobenzyl)amino)ethyl)-4-(trifluoromethoxy)benzenesulfonamide;
N-((4-fluorophenyl)(tetrahydrofuran-2-yl)methyl)-4-(trifluoromethoxy)benzenesulfonamide;
N-(1-(4-chlorophenyl)-2-(piperidin-1-yl)ethyl)-4-(trifluoromethoxy)benzenesulfonamide;
N-(1-(3,4-dichlorophenyl)-2-(dimethylamino)ethyl)-4-ethylbenzenesulfonamide;
N-(1-(2,5-difluorophenyl)propyl)-4-(trifluoromethoxy)benzenesulfonamide;
methyl 3-(3,5-dichlorophenyl)-3-((4-(trifluoromethoxy)phenyl)sulfonamido)propanoate;
N-((3-chlorophenyl)(cyclopentyl)methyl)-4-(trifluoromethoxy)benzenesulfonamide;
4-(trifluoromethoxy)-N-((1-(3-(trifluoromethyl)phenyl)cyclobutyl)methyl)benzenesulfonamide;
N-(1-(benzo[d][1,3]dioxol-5-yl)butyl)-4-(trifluoromethoxy)benzenesulfonamide;
methyl 3-(3-fluoro-4-hydroxyphenyl)-2-((4-(trifluoromethoxy)phenyl)sulfonamido)propanoate;
N-(5-chloro-2-ethoxyphenethyl)-4-(trifluoromethoxy)benzenesulfonamide;
N-((2-phenylcyclohexyl)methyl)-4-(trifluoromethoxy)benzenesulfonamide;
methyl 3-(3-bromo-4-methoxyphenyl)-3-((4-(trifluoromethoxy)phenyl)sulfonamido)propanoate;
tert-butyl 2-((4-(trifluoromethoxy)phenyl)sulfonamido)hexanoate;
N-((1-(4-bromobenzyl)cyclopropyl)methyl)-4-(trifluoromethoxy)benzenesulfonamide;
N-(cyclopropyl(4-fluoro-3-(trifluoromethyl)phenyl)methyl)-4-(trifluoromethoxy)benzenesulfonamide;
N-(1-(3,4-dichlorophenyl)-2-hydroxyethyl)-4-(trifluoromethoxy)benzenesulfonamide;
N-(3-(2,5-dimethoxyphenyl)propyl)-4-(trifluoromethoxy)benzenesulfonamide;
N-(1-(3,4-dichlorophenyl)-2-(dimethylamino)ethyl)-3-methylbenzenesulfonamide;
4-chloro-2-fluoro-N-(2-((4-(trifluoromethoxy)phenyl)sulfonamido)ethyl)benzenesulfonamide;
methyl 2-(2-bromophenyl)-2-((4-(trifluoromethoxy)phenyl)sulfonamido)acetate;
tert-butyl 4,4-difluoro-2-(((4-(trifluoromethoxy)phenyl)sulfonamido)methyl)pyrrolidine-1-carboxylate;
N-(2-bromobenzyl)-N-methyl-3-((4-(trifluoromethoxy)phenyl)sulfonamido)propanamide;
N-((1-(2,4-dimethylphenyl)cyclopropyl)methyl)-4-(trifluoromethoxy)benzenesulfonamide;
N-(1-(3-chlorophenyl)-2-(dimethylamino)ethyl)-4-(trifluoromethoxy)benzenesulfonamide;
N-(2-morpholino-2-(4-(trifluoromethyl)phenyl)ethyl)-4-(trifluoromethoxy)benzenesulfonamide
N-(2-(dimethylamino)-1-(p-tolyl)ethyl)-4-(trifluoromethoxy)benzenesulfonamide
N-(1-(3,4-dichlorophenyl)-2-(dimethylamino)ethyl)-4-(difluoromethoxy)benzenesulfonamide;
N-(2-(furan-2-yl)-2-(4-methylpiperidin-1-yl)ethyl)-4-(trifluoromethoxy)benzenesulfonamide;
ethyl 3-(2-chlorophenyl)-2-((4-(trifluoromethoxy)phenyl)sulfonamido)propanoate;
N-(2-(tert-butoxy)benzyl)-4-(trifluoromethoxy)benzenesulfonamide;
3-chloro-N-(1-(3,4-dichlorophenyl)-2-(dimethylamino)ethyl)benzenesulfonamide;
N-(2-(dimethylamino)-1-(3-(trifluoromethyl)phenyl)ethyl)-4-(trifluoromethoxy)benzenesulfonamide;
N-(1-(3,4-dichlorophenyl)-2-(dimethylamino)ethyl)-4-(trifluoromethoxy)benzenesulfonimidamide;
N-(1-(5-fluoro-2-(4-hydroxypiperidin-1-yl)phenyl)ethyl)-4-(trifluoromethoxy)benzenesulfonamide;
N-(2-(dimethylamino)-1-(m-tolyl)ethyl)-4-(trifluoromethoxy)benzenesulfonamide;
ethyl 3-(3-bromophenyl)-3-((4-(trifluoromethoxy)phenyl)sulfonamido)propanoate;
N-(1-(3,4-dichlorophenyl)-2-(dimethylamino)ethyl)-2-methylbenzenesulfonamide;
4-chloro-N-(1-(3,4-dichlorophenyl)-2-(dimethylamino)ethyl)-3-fluorobenzenesulfonamide;
ethyl 3-(3-chlorophenyl)-2-((4-(trifluoromethoxy)phenyl)sulfonamido)propanoate;
N-(1-(2-bromo-4-fluorophenyl)-2-cyclopropylethyl)-4-(trifluoromethoxy)benzenesulfonamide;
N-(2-(pyrrolidin-1-yl)benzyl)-4-(trifluoromethoxy)benzenesulfonamide;
ethyl 2-(3-bromo-4-methoxyphenyl)-2-((4-(trifluoromethoxy)phenyl)sulfonamido)acetate;
methyl 3-(2-hydroxyphenyl)-2-((4-(trifluoromethoxy)phenyl)sulfonamido)propanoate;
N-(1-(3,4-dichlorophenyl)-3-methoxypropyl)-4-(trifluoromethoxy)benzenesulfonamide;
N-(1-(2-chlorophenyl)-1-methoxypropan-2-yl)-4-(trifluoromethoxy)benzenesulfonamide;
N-(1-(3,4-dichlorophenyl)-2-(dimethylamino)ethyl)-4-ethoxybenzenesulfonamide;
N-(1-(4-chlorophenyl)-2-(pyrrolidin-1-yl)ethyl)-4-(trifluoromethoxy)benzenesulfonamide;
N-(1-(3,4-dichlorophenyl)-2-(dimethylamino)ethyl)-4-methylbenzenesulfonamide;
N′-(1-(3,4-dichlorophenyl)-2-(dimethylamino)ethyl)-N-methyl-4-(trifluoromethoxy)benzenesulfonimidamide;
N-(1-(3,4-dichlorophenyl)-2-(dimethylamino)ethyl)-3-(trifluoromethyl)benzenesulfonamide;
ethyl 3-(cyclohex-3-en-1-yl)-2-((4-(trifluoromethoxy)phenyl)sulfonamido)propanoate;
N-cyclohexyl-4-(N-(1-(3,4-dichlorophenyl)-2-(dimethylamino)ethyl)sulfamoyl)benzamide;
N-(1-(3,4-dichlorophenyl)-2-morpholinoethyl)-4-(trifluoromethoxy)benzenesulfonamide;
N-cycloheptyl-N-methyl-2-((4-(trifluoromethoxy)phenyl)sulfonamido)pentanamide;
N-(4,4,4-trifluoro-3-phenylbutyl)-4-(trifluoromethoxy)benzenesulfonamide;
N-(1-(5-ethylfuran-2-yl)-2,2-dimethylpropyl)-4-(trifluoromethoxy)benzenesulfonamide;
N-(2-(4-ethylphenyl)-2-(pyrrolidin-1-yl)ethyl)-4-(trifluoromethoxy)benzenesulfonamide;
N-(2-(isopentyloxy)phenethyl)-4-(trifluoromethoxy)benzenesulfonamide;
N-(1-(4-chlorophenyl)-2-(dimethylamino)ethyl)-4-(trifluoromethoxy)benzenesulfonamide;
methyl 3-(3,4-dichlorophenyl)-2-((4-(trifluoromethoxy)phenyl)sulfonamido)propanoate;
N-(1-(3,4-dichlorophenyl)-2-(dimethylamino)ethyl)cyclohexanesulfonamide;
ethyl 3-(2,5-dichlorothiophen-3-yl)-2-((4-(trifluoromethoxy)phenyl)sulfonamido)propanoate;
tert-butyl 3-methyl-2-((4-(trifluoromethoxy)phenyl)sulfonamido)pentanoate;
N-(2-(piperidin-1-ylsulfonyl)benzyl)-4-(trifluoromethoxy)benzenesulfonamide;
4-chloro-N-(1-(3,4-dichlorophenyl)-2-(dimethylamino)ethyl)-2-fluorobenzenesulfonamide;
N-(1-(3-bromophenyl)-4,4,4-trifluorobutan-2-yl)-4-(trifluoromethoxy)benzenesulfonamide;
N-(2-(dimethylamino)-1-(o-tolyl)ethyl)-4-(trifluoromethoxy)benzenesulfonamide;
N-(2-ethoxybenzyl)-4-(trifluoromethoxy)benzenesulfonamide;
N-(2-isobutoxy-4-methylbenzyl)-4-(trifluoromethoxy)benzenesulfonamide;
ethyl 3-(tetrahydrofuran-3-yl)-2-((4-(trifluoromethoxy)phenyl)sulfonamido)propanoate;
methyl 4-phenyl-3-((4-(trifluoromethoxy)phenyl)sulfonamido)butanoate;
N-(1-(3,4-dichlorophenyl)-2-(dimethylamino)ethyl)-4-fluorobenzenesulfonamide;
N-((1-((3,4-dimethoxyphenyl)sulfonyl)piperidin-4-yl)methyl)-4-(trifluoromethoxy)benzenesulfonamide;
N-(3-(methylsulfonyl)-5-(trifluoromethyl)benzyl)-4-(trifluoromethoxy)benzenesulfonamide;
4-nitro-N-(2-((4-(trifluoromethoxy)phenyl)sulfonamido)ethyl)benzenesulfonamide;
3-(4-isopropylphenyl)-3-((4-(trifluoromethoxy)phenyl)sulfonamido)propanoic acid;
methyl 2-phenyl-2-((4-(trifluoromethoxy)phenyl)sulfonamido)butanoate;
N-(1-(3,4-dichlorophenyl)-2-(dimethylamino)ethyl)-3-fluorobenzenesulfonamide;
N-(5-chloro-2-(difluoromethoxy)benzyl)-4-(trifluoromethoxy)benzenesulfonamide;
ethyl 4-methyl-2-((4-(trifluoromethoxy)phenyl)sulfonamido)hexanoate;
N-(3-(1-cyanopropoxy)benzyl)-4-(trifluoromethoxy)benzenesulfonamide;
ethyl 3-(4-methoxyphenyl)-2-((4-(trifluoromethoxy)phenyl)sulfonamido)propanoate;
ethyl 3-(4-isopropoxy-3-methoxyphenyl)-3-((4-(trifluoromethoxy)phenyl)sulfonamido)propanoate;
N-((1-(4-(difluoromethoxy)-3-methoxyphenyl)cyclopentyl)methyl)-4-(trifluoromethoxy)benzenesulfonamide;
N-((4-chlorophenyl)(cyclopentyl)methyl)-4-(trifluoromethoxy)benzenesulfonamide;
ethyl 3-(4-ethoxyphenyl)-3-((4-(trifluoromethoxy)phenyl)sulfonamido)propanoate;
4-cyclopropoxy-N-(1-(3,4-dichlorophenyl)-2-(dimethylamino)ethyl)benzenesulfonamide;
N-(1-(3-chloro-4-fluorophenyl)-2-(dimethylamino)ethyl)-4-(trifluoromethoxy)benzenesulfonamide;
N-(1-(4-chloro-3-fluorophenyl)-2-(dimethylamino)ethyl)-4-(trifluoromethoxy)benzenesulfonamide;
4-(N-(1-(3,4-dichlorophenyl)-2-(dimethylamino)ethyl)sulfamoyl)-N-phenylbenzamide;
N-(1-(3,4-dichlorophenyl)-2-(dimethylamino)ethyl)-4-(phenylamino)benzenesulfonamide;
N-(1-(4-chlorophenyl)-2-(dimethylamino)ethyl)-4-(4-(trifluoromethyl)phenoxy)benzenesulfonamide;
N-(2-(dimethylamino)-1-(3-(trifluoromethyl)phenyl)ethyl)-3-nitro-4-(trifluoromethoxy)benzenesulfonamide;
N-(1-(4-chlorophenyl)-2-(dimethylamino)ethyl)-3-nitro-4-(trifluoromethoxy)benzenesulfonamide;
4-(cyclopropylmethoxy)-N-(1-(3,4-dichlorophenyl)-2-(dimethylamino)ethyl)benzenesulfonamide
N-(1-(3,4-dichlorophenyl)-2-(dimethylamino)ethyl)-4-isopropoxybenzenesulfonamide;
4-(cyclohexyloxy)-N-(1-(3,4-dichlorophenyl)-2-(dimethylamino)ethyl)benzenesulfonamide;
N-(1-(3,4-dichlorophenyl)-2-(dimethylamino)ethyl)-4-(pyridin-2-yloxy)benzenesulfonamide;
4-butyl-N-(1-(3,4-dichlorophenyl)-2-(dimethylamino)ethyl)benzenesulfonamide;
N-(1-(3,4-dichlorophenyl)-2-(dimethylamino)ethyl)-4-(3,6-dihydro-2H-pyran-4-yl)benzenesulfonamide;
N-(2-(dimethylamino)-1-(4-(trifluoromethyl)phenyl)ethyl)-4-(trifluoromethoxy)benzenesulfonamide;
N-(1-(3,4-dichlorophenyl)-2-(dimethylamino)ethyl)-4-(3-fluorophenoxy)benzenesulfonamide;
N-(1-(3,4-dichlorophenyl)-2-(dimethylamino)ethyl)-4-(4-fluorophenoxy)benzenesulfonamide;
methyl 5-(N-(1-(3,4-dichlorophenyl)-2-(dimethylamino)ethyl)sulfamoyl)-2-(trifluoromethoxy)benzoate;
N-(1-(3,4-dichlorophenyl)-2-(4-methylpiperazin-1-yl)ethyl)-4-(4-(trifluoromethyl)phenoxy)benzenesulfonamide;
N-(1-(4-chlorophenyl)-2-(4-methylpiperazin-1-yl)ethyl)-4-phenoxybenzenesulfonamide;
N-(1-(4-chlorophenyl)-2-(4-methylpiperazin-1-yl)ethyl)-4-(4-(trifluoromethyl)phenoxy)benzenesulfonamide;
N-(1-(3,4-dichlorophenyl)-2-(4-methylpiperazin-1-yl)ethyl)-4-phenoxybenzenesulfonamide;
N-(2-(dimethylamino)-1-(3-(trifluoromethyl)phenyl)ethyl)-4-(4-(trifluoromethyl)phenoxy)benzenesulfonamide;
N-(1-(3,4-dichlorophenyl)-2-(dimethylamino)ethyl)-6-(trifluoromethoxy)pyridine-3-sulfonamide;
N-(1-(3,4-dichlorophenyl)-2-(dimethylamino)ethyl)-3-nitro-4-(trifluoromethoxy)benzenesulfonamide;
N-(1-(3,4-dichlorophenyl)-2-(dimethylamino)ethyl)-4-(pyridin-2-yl)benzenesulfonamide;
N-(1-(3,4-dichlorophenyl)-2-(dimethylamino)ethyl)-4-nitro-3-(trifluoromethoxy)benzenesulfonamide;
N-(2-amino-1-(3,4-dichlorophenyl)ethyl)-4-(trifluoromethoxy)benzenesulfonamide hydrochloride;
tert-butyl (2-(3,4-dichlorophenyl)-2-((4-(trifluoromethoxy)phenyl)sulfonamido)ethyl)carbamate;
N-((1-(4-chlorophenyl)cyclopropyl)methyl)-4-(trifluoromethoxy)benzenesulfonamide;
N-((1-(4-chlorophenyl)cyclobutyl)methyl)-4-(trifluoromethoxy)benzenesulfonamide;
N-((2,2-dimethyl-1-phenylcyclopropyl)methyl)-4-(trifluoromethoxy)benzenesulfonamide;
N-(1-(3,4-dichlorophenyl)-2-(dimethylamino)ethyl)-2-nitro-3-(trifluoromethoxy)benzenesulfonamide;
N-((1-(3,4-dichlorophenyl)cyclopropyl)methyl)-4-(trifluoromethoxy)benzenesulfonamide;
N-((1-(3,4-dichlorophenyl)cyclopropyl)methyl)-4-(4-(trifluoromethyl)phenoxy)benzenesulfonamide;
N-((1-(3,4-dichlorophenyl)cyclopropyl)methyl)-3-nitro-4-(trifluoromethoxy)benzenesulfonamide;
N-(1-(3,4-dichlorophenyl)-2-(piperazin-1-yl)ethyl)-4-(trifluoromethoxy)benzenesulfonamide;
N-(1-(3,4-dichlorophenyl)-2-(dimethylamino)ethyl)-3,5-difluorobenzenesulfonamide;
N-(1-(2-methoxy-5-methylphenyl)-3-methylbutan-2-yl)-4-(trifluoromethoxy)benzenesulfonamide;
N-(1-(3,4-dichlorophenyl)-2-(dimethylamino)ethyl)-4-(2-(trifluoromethyl)phenoxy)benzenesulfonamide;
N-(1-(3,4-dichlorophenyl)-2-(dimethylamino)ethyl)-4-(tetrahydro-2H-pyran-2-yl)benzenesulfonamide;
3-cyano-N-(1-(3,4-dichlorophenyl)-2-(dimethylamino)ethyl)-4-(trifluoromethoxy)benzenesulfonamide;
N-(1-(3,4-dichlorophenyl)-2-(4-propoxypiperidin-1-yl)ethyl)-4-(trifluoromethoxy)benzenesulfonamide;
N-(1-(3,4-dichlorophenyl)-2-(methylamino)ethyl)-4-(trifluoromethoxy)benzenesulfonamide;
N-((1-(3,4-dichlorophenyl)cyclopentyl)methyl)-4-(trifluoromethoxy)benzenesulfonamide;
N-((1-(4-chlorophenyl)cyclopentyl)methyl)-4-(trifluoromethoxy)benzenesulfonamide;
N-(2-(1-(3,4-dichlorophenyl)cyclopropyl)propan-2-yl)-4-(trifluoromethoxy)benzenesulfonamide;
N-(1-(3,4-dichlorophenyl)-3-(dimethylamino)propan-2-yl)-4-(trifluoromethoxy)benzenesulfonamide;
4-cyclobutoxy-N-(1-(3,4-dichlorophenyl)-2-(dimethylamino)ethyl)benzenesulfonamide;
3-(3,4-dichlorophenyl)-N,N-dimethyl-2-((4-(trifluoromethoxy)phenyl)sulfonamido)propenamide;
N-(1-(3,4-dichlorophenyl)-2-(dimethylamino)ethyl)-N-methyl-4-(trifluoromethoxy)benzenesulfonamide;
4-(cyclopentylamino)-N-(1-(3,4-dichlorophenyl)-2-(dimethylamino)ethyl)benzenesulfonamide;
4-(cyclobutylamino)-N-(1-(3,4-dichlorophenyl)-2-(dimethylamino)ethyl)benzenesulfonamide;
N-((3-(3,4-dichlorophenyl)pyrrolidin-3-yl)methyl)-4-(trifluoromethoxy)benzenesulfonamide;
2-(3,4-dichlorophenyl)-N,N-dimethyl-2-((4-(trifluoromethoxy)phenyl)sulfonamido)acetamide;
N-(1-(3,4-dichlorophenyl)-2-((4-methoxybenzyl)amino)ethyl)-4-(trifluoromethoxy)benzenesulfonamide;
N-(1-(4-chlorophenyl)-2-(piperazin-1-yl)ethyl)-4-(trifluoromethoxy)benzenesulfonamide;
N-(1-(4-chloro-3-fluorophenyl)-2-(piperazin-1-yl)ethyl)-4-(trifluoromethoxy)benzenesulfonamide;
N-(1-(4-chlorophenyl)-2-(piperazin-1-yl)ethyl)-6-(trifluoromethoxy)pyridine-3-sulfonamide;
N-((4-phenylpiperidin-4-yl)methyl)-4-(trifluoromethoxy)benzenesulfonamide;
N-(2-(2,5-diazabicyclo[2.2.1]heptan-2-yl)-1-(3,4-dichlorophenyl)ethyl)-4-(trifluoromethoxy)benzenesulfonamide;
4-(trifluoromethoxy)-N-((1-(3-(trifluoromethyl)phenyl)cyclopentyl)methyl)benzenesulfonamide;
N-((2-phenylpyrrolidin-2-yl)methyl)-4-(trifluoromethoxy)benzenesulfonamide;
N-(1-(4-fluorophenyl)-2-(piperazin-1-yl)ethyl)-4-(trifluoromethoxy)benzenesulfonamide;
N-(2-(4-fluorophenyl)-2-(piperazin-1-yl)ethyl)-4-(trifluoromethoxy)benzenesulfonamide;
N-(1-(3,4-dichlorophenyl)-2-(dimethylamino)ethyl)-3-nitro-4-(trifluoromethyl)benzenesulfonamide;
N-(1-(3,4-dichlorophenyl)-2-(dimethylamino)ethyl)-4-propoxybenzenesulfonamide;
4-(cyclohexylamino)-N-(1-(3,4-dichlorophenyl)-2-(dimethylamino)ethyl)benzenesulfonamide; and
N-(1-(3,4-dichlorophenyl)-2-(4-methylpiperazin-1-yl)ethyl)-4-(4-fluorophenoxy)benzenesulfonamide;
or a salt, solvate, isotopologue, or tautomer thereof.

In certain embodiments, the compound of formula (I) is selected from the group consisting of
(R)—N-(1-(3,4-dichlorophenyl)-2-(dimethylamino)ethyl)-3-nitro-4-(trifluoromethoxy)benzenesulfonamide;
(R)—N-(1-hydroxy-5-methylhexan-3-yl)-4-(trifluoromethoxy)benzenesulfonamide;
(R)—N-(1-(3,4-dichlorophenyl)-2-(dimethylamino)ethyl)-4-pentylbenzenesulfonamide;
(R)—N-(cyclohexyl(3,5-dichlorophenyl)methyl)-4-(trifluoromethoxy)benzenesulfonamide;
(R)—N-(1-(3,4-dichlorophenyl)-2-(dimethylamino)ethyl)-4-(4-(trifluoromethyl)phenoxy)benzenesulfonamide;
(R)—N-(1-(3,4-dichlorophenyl)-2-(dimethylamino)ethyl)-4-phenoxybenzenesulfonamide;
(R)—N-(1-(3,4-dichlorophenyl)-2-(dimethylamino)ethyl)-[1,1′-biphenyl]-4-sulfonamide;
(R)—N-(1-(4-chlorophenyl)-2-(dimethylamino)ethyl)-4-phenoxybenzenesulfonamide;
(R)—N-(2-(dimethylamino)-1-(3-(trifluoromethyl)phenyl)ethyl)-4-phenoxybenzenesulfonamide;
(R)-3-chloro-N-(1-(3,4-dichlorophenyl)-2-(dimethylamino)ethyl)-4-(trifluoromethoxy)benzenesulfonamide;
(R)-4-(cyclopentyloxy)-N-(1-(3,4-dichlorophenyl)-2-(dimethylamino)ethyl)benzenesulfonamide;
(R)—N-(1-(3,4-dichlorophenyl)-2-(dimethylamino)ethyl)-4-propylbenzenesulfonamide;
(R)—N-(1-(3,4-dichlorophenyl)-2-(4-methylpiperazin-1-yl)ethyl)-4-(trifluoromethoxy)benzenesulfonamide;
(R)—N-(1-(3,4-dichlorophenyl)-2-(dimethylamino)ethyl)-[1,1′-biphenyl]-3-sulfonamide;
(R)-3-chloro-N-(1-(4-chlorophenyl)-2-(dimethylamino)ethyl)-4-(trifluoromethoxy)benzenesulfonamide;
(R)-3-chloro-N-(2-(dimethylamino)-1-(3-(trifluoromethyl)phenyl)ethyl)-4-(trifluoromethoxy)benzenesulfonamide;
(R)—N-(1-(3-bromo-5-fluorophenyl)-2-cyclopropylethyl)-4-(trifluoromethoxy)benzenesulfonamide;
(R)—N-((1-(2,4-difluorophenyl)-2,2-dimethylcyclopropyl)methyl)-4-(trifluoromethoxy)benzenesulfonamide;
(R)—N-(1-(3,4-dichlorophenyl)-2-(dimethylamino)ethyl)-4-(trifluoromethoxy)benzenesulfonamide;
(R)—N-(2-(dimethylamino)-1-(3-(trifluoromethyl)phenyl)ethyl)-4-(4-(trifluoromethyl)phenoxy)benzenesulfonamide;
(R)—N-(2-(isopropyl(methyl)amino)benzyl)-4-(trifluoromethoxy)benzenesulfonamide;
(R)-4-(trifluoromethoxy)-N-(1-(4-(trifluoromethyl)phenyl)pent-4-en-1-yl)benzenesulfonamide;
N—((R)-((1R,2R)-2-methylcyclopropyl)(phenyl)methyl)-4-(trifluoromethoxy)benzenesulfonamide;
N—((R)-((1R,2S)-2-methylcyclopropyl)(phenyl)methyl)-4-(trifluoromethoxy)benzenesulfonamide;
(R)—N-(1-(3,4-dichlorophenyl)-2-(dimethylamino)ethyl)-3-(trifluoromethoxy)benzenesulfonamide;
(R)-2-amino-N-(1-(3,4-dichlorophenyl)-2-(dimethylamino)ethyl)-4-(trifluoromethoxy)benzenesulfonamide;
(R)—N-(1-(3,4-dichlorophenyl)-2-(dimethylamino)ethyl)naphthalene-1-sulfonamide;
(R)-2-chloro-N-(1-(3,4-dichlorophenyl)-2-(dimethylamino)ethyl)-4-(trifluoromethoxy)benzenesulfonamide;
(R)—N-(1-(3,4-dichlorophenyl)-2-(dimethylamino)ethyl)naphthalene-2-sulfonamide;
(R)-3,4-dichloro-N-(1-(3,4-dichlorophenyl)-2-(dimethylamino)ethyl)benzenesulfonamide;
N-(((2R,3R)-2-(4-methoxyphenyl)-1-methylpyrrolidin-3-yl)methyl)-4-(trifluoromethoxy)benzenesulfonamide;
N-(((2R,3S)-2-(4-methoxyphenyl)-1-methylpyrrolidin-3-yl)methyl)-4-(trifluoromethoxy)benzenesulfonamide;
(R)—N-(1-hydroxy-3-(m-tolyl)propan-2-yl)-4-(trifluoromethoxy)benzenesulfonamide;
(R)—N-(1-(3,4-dichlorophenyl)-2-(dimethylamino)ethyl)-2-phenylethane-1-sulfonamide;
(R)—N-(1-(3,4-dichlorophenyl)-2-(pyrrolidin-1-yl)ethyl)-4-(trifluoromethoxy)benzenesulfonamide;
(R)—N-(1-(3,4-dichlorophenyl)-2-(dimethylamino)ethyl)-N′-methyl-4-(trifluoromethoxy)benzenesulfonimidamide;
(R)—N-(1-(3,4-dichlorophenyl)-2-(dimethylamino)ethyl)-2,2-difluorobenzo[d] [1,3]dioxole-5-sulfonamide;
(R)-ethyl 3-((4-(trifluoromethoxy)phenyl)sulfonamido)-3-(3-(trifluoromethyl)phenyl)propanoate;
(R)—N-(1-(1-(4-bromophenyl)cyclopropyl)ethyl)-4-(trifluoromethoxy)benzenesulfonamide;
(R)-4-methyl-N-(thiazol-2-yl)-2-((4-(trifluoromethoxy)phenyl)sulfonamido)pentanamide;
(R)—N-(1-(3,4-dichlorophenyl)-2-(dimethylamino)ethyl)-4-isopropylbenzenesulfonamide;
(R)—N-(1-(3,4-dichlorophenyl)-2-(dimethylamino)ethyl)-4-(trifluoromethyl)benzenesulfonamide;
(R)—N-(4,4,4-trifluoro-1-(2,4,5-trifluorophenyl)butan-2-yl)-4-(trifluoromethoxy)benzenesulfonamide;
(R)—N-((6-benzyl-6-azaspiro[3.4]octan-8-yl)methyl)-4-(trifluoromethoxy)benzenesulfonamide;
(R)-4-chloro-N-(1-(3,4-dichlorophenyl)-2-(dimethylamino)ethyl)benzenesulfonamide;
(R)—N-(1-(3-cyanophenyl)-3-methylbutyl)-4-(trifluoromethoxy)benzenesulfonamide;
(R)—N-(cyclohexyl(3-(trifluoromethyl)phenyl)methyl)-4-(trifluoromethoxy)benzenesulfonamide;
(R,E)-N-(1-(3,4-dichlorophenyl)-2-(dimethylamino)ethyl)-2-phenylethene-1-sulfonamide;
(R)—N-(2-(2-hydroxypropoxy)benzyl)-4-(trifluoromethoxy)benzenesulfonamide;
(R)-3-amino-N-(1-(3,4-dichlorophenyl)-2-(dimethylamino)ethyl)-4-(trifluoromethoxy)benzenesulfonamide;
N-((1S)-(4-fluorophenyl)(tetrahydrofuran-2-yl)methyl)-4-(trifluoromethoxy)benzenesulfonamide;
(R)—N-(1-(4-chlorophenyl)-2-(piperidin-1-yl)ethyl)-4-(trifluoromethoxy)benzenesulfonamide;
N—((R)-1-((R)-1-methylpiperidin-2-yl)propan-2-yl)-4-(trifluoromethoxy)benzenesulfonamide;
N—((R)-1-((S)-1-methylpiperidin-2-yl)propan-2-yl)-4-(trifluoromethoxy)benzenesulfonamide;
(R)—N-(1-(3,4-dichlorophenyl)-2-(dimethylamino)ethyl)-4-ethylbenzenesulfonamide;
(R)—N-(1-(2,5-difluorophenyl)propyl)-4-(trifluoromethoxy)benzenesulfonamide;
methyl (R)-3-(3,5-dichlorophenyl)-3-((4-(trifluoromethoxy)phenyl)sulfonamido)propanoate;
(R)—N-((3-chlorophenyl)(cyclopentyl)methyl)-4-(trifluoromethoxy)benzenesulfonamide;
(R)—N-(1-(benzo[d][1,3]dioxol-5-yl)butyl)-4-(trifluoromethoxy)benzenesulfonamide;
methyl (R)-3-(3-fluoro-4-hydroxyphenyl)-2-((4-(trifluoromethoxy)phenyl)sulfonamido)propanoate;
methyl (R)-3-(3-bromo-4-methoxyphenyl)-3-((4-(trifluoromethoxy)phenyl)sulfonamido)propanoate;
tert-butyl (R)-2-((4-(trifluoromethoxy)phenyl)sulfonamido)hexanoate;
(R)—N-(cyclopropyl(4-fluoro-3-(trifluoromethyl)phenyl)methyl)-4-(trifluoromethoxy)benzenesulfonamide;
(R)—N-(1-(3,4-dichlorophenyl)-2-hydroxyethyl)-4-(trifluoromethoxy)benzenesulfonamide;
(R)—N-(1-(3,4-dichlorophenyl)-2-(dimethylamino)ethyl)-3-methylbenzenesulfonamide;
methyl (R)-2-(2-bromophenyl)-2-((4-(trifluoromethoxy)phenyl)sulfonamido)acetate;
tert-butyl (R)-4,4-difluoro-2-(((4-(trifluoromethoxy)phenyl)sulfonamido)methyl)pyrrolidine-1-carboxylate;
(R)—N-(1-(3-chlorophenyl)-2-(dimethylamino)ethyl)-4-(trifluoromethoxy)benzenesulfonamide;
(R)—N-(2-morpholino-2-(4-(trifluoromethyl)phenyl)ethyl)-4-(trifluoromethoxy)benzenesulfonamide;
(R)—N-(2-(dimethylamino)-1-(p-tolyl)ethyl)-4-(trifluoromethoxy)benzenesulfonamide;
(R)—N-(1-(3,4-dichlorophenyl)-2-(dimethylamino)ethyl)-4-(difluoromethoxy)benzenesulfonamide;
(R)—N-(2-(furan-2-yl)-2-(4-methylpiperidin-1-yl)ethyl)-4-(trifluoromethoxy)benzenesulfonamide;
ethyl (R)-3-(2-chlorophenyl)-2-((4-(trifluoromethoxy)phenyl)sulfonamido)propanoate;
(R)—N-(2-(tert-butoxy)benzyl)-4-(trifluoromethoxy)benzenesulfonamide;
(R)-3-chloro-N-(1-(3,4-dichlorophenyl)-2-(dimethylamino)ethyl)benzenesulfonamide;
(R)—N-(2-(dimethylamino)-1-(3-(trifluoromethyl)phenyl)ethyl)-4-(trifluoromethoxy)benzenesulfonamide;
(R,R)—N-(1-(3,4-dichlorophenyl)-2-(dimethylamino)ethyl)-4-(trifluoromethoxy)benzenesulfonimidamide;
(R,S)—N-(1-(3,4-dichlorophenyl)-2-(dimethylamino)ethyl)-4-(trifluoromethoxy)benzenesulfonimidamide;
(R)—N-(1-(5-fluoro-2-(4-hydroxypiperidin-1-yl)phenyl)ethyl)-4-(trifluoromethoxy)benzenesulfonamide;
(R)—N-(2-(dimethylamino)-1-(m-tolyl)ethyl)-4-(trifluoromethoxy)benzenesulfonamide;
ethyl (R)-3-(3-bromophenyl)-3-((4-(trifluoromethoxy)phenyl)sulfonamido)propanoate;
(R)—N-(1-(3,4-dichlorophenyl)-2-(dimethylamino)ethyl)-2-methylbenzenesulfonamide;
(R)-4-chloro-N-(1-(3,4-dichlorophenyl)-2-(dimethylamino)ethyl)-3-fluorobenzenesulfonamide;
ethyl (R)-3-(3-chlorophenyl)-2-((4-(trifluoromethoxy)phenyl)sulfonamido)propanoate;
(R)—N-(1-(2-bromo-4-fluorophenyl)-2-cyclopropylethyl)-4-(trifluoromethoxy)benzenesulfonamide;
ethyl (R)-2-(3-bromo-4-methoxyphenyl)-2-((4-(trifluoromethoxy)phenyl)sulfonamido)acetate;
methyl (R)-3-(2-hydroxyphenyl)-2-((4-(trifluoromethoxy)phenyl)sulfonamido)propanoate;
(R)—N-(1-(3,4-dichlorophenyl)-3-methoxypropyl)-4-(trifluoromethoxy)benzenesulfonamide;
(R)—N-(1-(3,4-dichlorophenyl)-2-(dimethylamino)ethyl)-4-ethoxybenzenesulfonamide;
(R)—N-(1-(4-chlorophenyl)-2-(pyrrolidin-1-yl)ethyl)-4-(trifluoromethoxy)benzenesulfonamide;
(R)—N-(1-(3,4-dichlorophenyl)-2-(dimethylamino)ethyl)-4-methylbenzenesulfonamide;
(R)—N′-(1-(3,4-dichlorophenyl)-2-(dimethylamino)ethyl)-N-methyl-4-(trifluoromethoxy)benzenesulfonimidamide;
(R)—N-(1-(3,4-dichlorophenyl)-2-(dimethylamino)ethyl)-3-(trifluoromethyl)benzenesulfonamide;
(R)—N-cyclohexyl-4-(N-(1-(3,4-dichlorophenyl)-2-(dimethylamino)ethyl)sulfamoyl)benzamide;
(R)—N-(1-(3,4-dichlorophenyl)-2-morpholinoethyl)-4-(trifluoromethoxy)benzenesulfonamide;
(R)—N-cycloheptyl-N-methyl-2-((4-(trifluoromethoxy)phenyl)sulfonamido)pentanamide;
(R)—N-(4,4,4-trifluoro-3-phenylbutyl)-4-(trifluoromethoxy)benzenesulfonamide;
(R)—N-(1-(5-ethylfuran-2-yl)-2,2-dimethylpropyl)-4-(trifluoromethoxy)benzenesulfonamide;
(R)—N-(2-(4-ethylphenyl)-2-(pyrrolidin-1-yl)ethyl)-4-(trifluoromethoxy)benzenesulfonamide;
(R)—N-(1-(4-chlorophenyl)-2-(dimethylamino)ethyl)-4-(trifluoromethoxy)benzenesulfonamide;
methyl (R)-3-(3,4-dichlorophenyl)-2-((4-(trifluoromethoxy)phenyl)sulfonamido)propanoate;
(R)—N-(1-(3,4-dichlorophenyl)-2-(dimethylamino)ethyl)cyclohexanesulfonamide;
ethyl (R)-3-(2,5-dichlorothiophen-3-yl)-2-((4-(trifluoromethoxy)phenyl)sulfonamido)propanoate;
tert-butyl ((4-(trifluoromethoxy)phenyl)sulfonyl)-D-isoleucinate;
tert-butyl ((4-(trifluoromethoxy)phenyl)sulfonyl)-D-alloisoleucinate
(R)-4-chloro-N-(1-(3,4-dichlorophenyl)-2-(dimethylamino)ethyl)-2-fluorobenzenesulfonamide;
(R)—N-(1-(3-bromophenyl)-4,4,4-trifluorobutan-2-yl)-4-(trifluoromethoxy)benzenesulfonamide;
(R)—N-(2-(dimethylamino)-1-(o-tolyl)ethyl)-4-(trifluoromethoxy)benzenesulfonamide;
methyl (R)-3-cyclohexyl-3-((4-(trifluoromethoxy)phenyl)sulfonamido)propanoate;
methyl (R)-4-phenyl-3-((4-(trifluoromethoxy)phenyl)sulfonamido)butanoate;
(R)—N-(1-(3,4-dichlorophenyl)-2-(dimethylamino)ethyl)-4-fluorobenzenesulfonamide;
(R)-3-(4-isopropylphenyl)-3-((4-(trifluoromethoxy)phenyl)sulfonamido)propanoic acid;
methyl (R)-2-phenyl-2-((4-(trifluoromethoxy)phenyl)sulfonamido)butanoate;
(R)—N-(1-(3,4-dichlorophenyl)-2-(dimethylamino)ethyl)-3-fluorobenzenesulfonamide;
ethyl (R)-4-methyl-2-((4-(trifluoromethoxy)phenyl)sulfonamido)hexanoate;
(R)—N-(3-(1-cyanopropoxy)benzyl)-4-(trifluoromethoxy)benzenesulfonamide;
ethyl (R)-3-(4-methoxyphenyl)-2-((4-(trifluoromethoxy)phenyl)sulfonamido)propanoate;
ethyl (R)-3-(4-isopropoxy-3-methoxyphenyl)-3-((4-(trifluoromethoxy)phenyl)sulfonamido)propanoate;
(R)—N-((4-chlorophenyl)(cyclopentyl)methyl)-4-(trifluoromethoxy)benzenesulfonamide;
ethyl (R)-3-(4-ethoxyphenyl)-3-((4-(trifluoromethoxy)phenyl)sulfonamido)propanoate;
(R)—N-(1-(3,4-dichlorophenyl)-2-(dimethylamino)ethyl)-3,5-difluorobenzenesulfonamide;
(R)—N-(1-(2-methoxy-5-methylphenyl)-3-methylbutan-2-yl)-4-(trifluoromethoxy)benzenesulfonamide;
N-(2-(((1R,2R)-2-methylcyclohexyl)oxy)ethyl)-4-(trifluoromethoxy)benzenesulfonamide;
N-(2-(((1R,2S)-2-methylcyclohexyl)oxy)ethyl)-4-(trifluoromethoxy)benzenesulfonamide;
N-(((1R,2R)-2-phenylcyclohexyl)methyl)-4-(trifluoromethoxy)benzenesulfonamide;
N-(((1R,2S)-2-phenylcyclohexyl)methyl)-4-(trifluoromethoxy)benzenesulfonamide;
N-(((1R,3R)-1-(dimethylamino)-3-methylcyclohexyl)methyl)-4-(trifluoromethoxy)benzenesulfonamide;
N-(((1R,3S)-1-(dimethylamino)-3-methylcyclohexyl)methyl)-4-(trifluoromethoxy)benzenesulfonamide;
N-((1R,2R)-2-(methylsulfonyl)-1-(p-tolyl)propyl)-4-(trifluoromethoxy)benzenesulfonamide;
N-((1R,2S)-2-(methylsulfonyl)-1-(p-tolyl)propyl)-4-(trifluoromethoxy)benzenesulfonamide;
N-((1R,2R)-1-(2-chlorophenyl)-1-methoxypropan-2-yl)-4-(trifluoromethoxy)benzenesulfonamide;
N-((1R,2S)-1-(2-chlorophenyl)-1-methoxypropan-2-yl)-4-(trifluoromethoxy)benzenesulfonamide;
ethyl (R)-3-((R)-tetrahydrofuran-3-yl)-2-((4-(trifluoromethoxy)phenyl)sulfonamido)propanoate;
ethyl (R)-3-((S)-tetrahydrofuran-3-yl)-2-((4-(trifluoromethoxy)phenyl)sulfonamido)propanoate;
ethyl (R)-3-((R)-cyclohex-3-en-1-yl)-2-((4-(trifluoromethoxy)phenyl)sulfonamido)propanoate;
ethyl (R)-3-((S)-cyclohex-3-en-1-yl)-2-((4-(trifluoromethoxy)phenyl)sulfonamido)propanoate;
N—((R)-(4-fluorophenyl)((R)-tetrahydrofuran-2-yl)methyl)-4-(trifluoromethoxy)benzenesulfonamide;
N—((R)-(4-fluorophenyl)((S)-tetrahydrofuran-2-yl)methyl)-4-(trifluoromethoxy)benzenesulfonamide;
(R)-4-cyclopropoxy-N-(1-(3,4-dichlorophenyl)-2-(dimethylamino)ethyl)benzenesulfonamide;
(R)—N-(1-(3-chloro-4-fluorophenyl)-2-(dimethylamino)ethyl)-4-(trifluoromethoxy)benzenesulfonamide;
(R)—N-(1-(4-chloro-3-fluorophenyl)-2-(dimethylamino)ethyl)-4-(trifluoromethoxy)benzenesulfonamide;
(R)-4-(N-(1-(3,4-dichlorophenyl)-2-(dimethylamino)ethyl)sulfamoyl)-N-phenylbenzamide;
(R)—N-(1-(3,4-dichlorophenyl)-2-(dimethylamino)ethyl)-4-(phenylamino)benzenesulfonamide;
(R)—N-(1-(4-chlorophenyl)-2-(dimethylamino)ethyl)-4-(4-(trifluoromethyl)phenoxy)benzenesulfonamide;
(R)—N-(2-(dimethylamino)-1-(3-(trifluoromethyl)phenyl)ethyl)-3-nitro-4-(trifluoromethoxy)benzenesulfonamide;
(R)—N-(1-(4-chlorophenyl)-2-(dimethylamino)ethyl)-3-nitro-4-(trifluoromethoxy)benzenesulfonamide;
(R)-4-(cyclopropylmethoxy)-N-(1-(3,4-dichlorophenyl)-2-(dimethylamino)ethyl)benzenesulfonamide;
(R)—N-(1-(3,4-dichlorophenyl)-2-(dimethylamino)ethyl)-4-isopropoxybenzenesulfonamide;
(R)-4-(cyclohexyloxy)-N-(1-(3,4-dichlorophenyl)-2-(dimethylamino)ethyl)benzenesulfonamide;
(R)—N-(1-(3,4-dichlorophenyl)-2-(dimethylamino)ethyl)-4-(pyridin-2-yloxy)benzenesulfonamide;
(R)-4-butyl-N-(1-(3,4-dichlorophenyl)-2-(dimethylamino)ethyl)benzenesulfonamide;
(R)—N-(1-(3,4-dichlorophenyl)-2-(dimethylamino)ethyl)-4-(3,6-dihydro-2H-pyran-4-yl)benzenesulfonamide;
(R)—N-(2-(dimethylamino)-1-(4-(trifluoromethyl)phenyl)ethyl)-4-(trifluoromethoxy)benzenesulfonamide;
(R)—N-(1-(3,4-dichlorophenyl)-2-(dimethylamino)ethyl)-4-(3-fluorophenoxy)benzenesulfonamide;
(R)—N-(1-(3,4-dichlorophenyl)-2-(dimethylamino)ethyl)-4-(4-fluorophenoxy)benzenesulfonamide;
(R)-methyl 5-(N-(1-(3,4-dichlorophenyl)-2-(dimethylamino)ethyl)sulfamoyl)-2-(trifluoromethoxy)benzoate;
(R)—N-(1-(3,4-dichlorophenyl)-2-(4-methylpiperazin-1-yl)ethyl)-4-(4-(trifluoromethyl)phenoxy)benzenesulfonamide;
(R)—N-(1-(4-chlorophenyl)-2-(4-methylpiperazin-1-yl)ethyl)-4-phenoxybenzenesulfonamide;
(R)—N-(1-(4-chlorophenyl)-2-(4-methylpiperazin-1-yl)ethyl)-4-(4-(trifluoromethyl)phenoxy)benzenesulfonamide;
(R)—N-(1-(3,4-dichlorophenyl)-2-(4-methylpiperazin-1-yl)ethyl)-4-phenoxybenzenesulfonamide;
(R)—N-(2-(dimethylamino)-1-(3-(trifluoromethyl)phenyl)ethyl)-4-(4-(trifluoromethyl)phenoxy)benzenesulfonamide;
(R)—N-(1-(3,4-dichlorophenyl)-2-(dimethylamino)ethyl)-6-(trifluoromethoxy)pyridine-3-sulfonamide;
(R)—N-(1-(3,4-dichlorophenyl)-2-(dimethylamino)ethyl)-3-nitro-4-(trifluoromethoxy)benzenesulfonamide;
(R)—N-(1-(3,4-dichlorophenyl)-2-(dimethylamino)ethyl)-4-(pyridin-2-yl)benzenesulfonamide;
(R)—N-(1-(3,4-dichlorophenyl)-2-(dimethylamino)ethyl)-4-nitro-3-(trifluoromethoxy)benzenesulfonamide;
(R)—N-(2-amino-1-(3,4-dichlorophenyl)ethyl)-4-(trifluoromethoxy)benzenesulfonamide hydrochloride;
(R)-tert-butyl (2-(3,4-dichlorophenyl)-2-((4-(trifluoromethoxy)phenyl)sulfonamido)ethyl)carbamate;
(R)—N-((2,2-dimethyl-1-phenylcyclopropyl)methyl)-4-(trifluoromethoxy)benzenesulfonamide;
(R)—N-(1-(3,4-dichlorophenyl)-2-(dimethylamino)ethyl)-2-nitro-3-(trifluoromethoxy)benzenesulfonamide;
4-(trifluoromethoxy)-N-((1R,3R)-3,4,4-trimethylcyclohexyl)benzenesulfonamide;
4-(trifluoromethoxy)-N-((1R,3S)-3,4,4-trimethylcyclohexyl)benzenesulfonamide;
(R)—N-(1-(3,4-dichlorophenyl)-2-(dimethylamino)ethyl)-4-(2-(trifluoromethyl)phenoxy)benzenesulfonamide;
(R)—N-(1-(3,4-dichlorophenyl)-2-(dimethylamino)ethyl)-4-(tetrahydro-2H-pyran-2-yl)benzenesulfonamide;
(R)-3-cyano-N-(1-(3,4-dichlorophenyl)-2-(dimethylamino)ethyl)-4-(trifluoromethoxy)benzenesulfonamide;
(R)—N-(1-(3,4-dichlorophenyl)-2-(4-propoxypiperidin-1-yl)ethyl)-4-(trifluoromethoxy)benzenesulfonamide;
(R)—N-(1-(3,4-dichlorophenyl)-2-(methylamino)ethyl)-4-(trifluoromethoxy)benzenesulfonamide;
(R)—N-(1-(3,4-dichlorophenyl)-3-(dimethylamino)propan-2-yl)-4-(trifluoromethoxy)benzenesulfonamide;
(R)-4-cyclobutoxy-N-(1-(3,4-dichlorophenyl)-2-(dimethylamino)ethyl)benzenesulfonamide;
(R)-3-(3,4-dichlorophenyl)-N,N-dimethyl-2-((4-(trifluoromethoxy)phenyl)sulfonamido)propenamide;
(R)—N-(1-(3,4-dichlorophenyl)-2-(dimethylamino)ethyl)-N-methyl-4-(trifluoromethoxy)benzenesulfonamide;
(R)-4-(cyclopentylamino)-N-(1-(3,4-dichlorophenyl)-2-(dimethylamino)ethyl)benzenesulfonamide;
(R)-4-(cyclobutylamino)-N-(1-(3,4-dichlorophenyl)-2-(dimethylamino)ethyl)benzenesulfonamide;
(R)—N-((3-(3,4-dichlorophenyl)pyrrolidin-3-yl)methyl)-4-(trifluoromethoxy)benzenesulfonamide;
(R)-2-(3,4-dichlorophenyl)-N,N-dimethyl-2-((4-(trifluoromethoxy)phenyl)sulfonamido)acetamide;
(R)—N-(1-(3,4-dichlorophenyl)-2-((4-methoxybenzyl)amino)ethyl)-4-(trifluoromethoxy)benzenesulfonamide;
(R)—N-(1-(4-chlorophenyl)-2-(piperazin-1-yl)ethyl)-4-(trifluoromethoxy)benzenesulfonamide;
(R)—N-(1-(4-chloro-3-fluorophenyl)-2-(piperazin-1-yl)ethyl)-4-(trifluoromethoxy)benzenesulfonamide;
(R)—N-(1-(4-chlorophenyl)-2-(piperazin-1-yl)ethyl)-6-(trifluoromethoxy)pyridine-3-sulfonamide;
N—((R)-2-((1R,4R)-2,5-diazabicyclo[2.2.1]heptan-2-yl)-1-(3,4-dichlorophenyl)ethyl)-4-(trifluoromethoxy)benzenesulfonamide;
N—((R)-2-((1S,4S)-2,5-diazabicyclo[2.2.1]heptan-2-yl)-1-(3,4-dichlorophenyl)ethyl)-4-(trifluoromethoxy)benzenesulfonamide;
(R)—N-((2-phenylpyrrolidin-2-yl)methyl)-4-(trifluoromethoxy)benzenesulfonamide;
(R)—N-(1-(4-fluorophenyl)-2-(piperazin-1-yl)ethyl)-4-(trifluoromethoxy)benzenesulfonamide;
(R)—N-(2-(4-fluorophenyl)-2-(piperazin-1-yl)ethyl)-4-(trifluoromethoxy)benzenesulfonamide;
(R)—N-(1-(3,4-dichlorophenyl)-2-(dimethylamino)ethyl)-3-nitro-4-(trifluoromethyl)benzenesulfonamide;
(R)—N-(1-(3,4-dichlorophenyl)-2-(dimethylamino)ethyl)-4-propoxybenzenesulfonamide;
(R)-4-(cyclohexylamino)-N-(1-(3,4-dichlorophenyl)-2-(dimethylamino)ethyl)benzenesulfonamide; and
(R)—N-(1-(3,4-dichlorophenyl)-2-(4-methylpiperazin-1-yl)ethyl)-4-(4-fluorophenoxy)benzenesulfonamide;
or a salt, solvate, isotopologue, or tautomer thereof.

In certain embodiments, the compound of formula (I) is selected from the group consisting of
(S)—N-(1-(3,4-dichlorophenyl)-2-(dimethylamino)ethyl)-3-nitro-4-(trifluoromethoxy)benzenesulfonamide;
(S)—N-(1-hydroxy-5-methylhexan-3-yl)-4-(trifluoromethoxy)benzenesulfonamide;
(S)—N-(1-(3,4-dichlorophenyl)-2-(dimethylamino)ethyl)-4-pentylbenzenesulfonamide;
(S)—N-(cyclohexyl(3,5-dichlorophenyl)methyl)-4-(trifluoromethoxy)benzenesulfonamide;
(S)—N-(1-(3,4-dichlorophenyl)-2-(dimethylamino)ethyl)-4-(4-(trifluoromethyl)phenoxy)benzenesulfonamide;
(S)—N-(1-(3,4-dichlorophenyl)-2-(dimethylamino)ethyl)-4-phenoxybenzenesulfonamide;
(S)—N-(1-(3,4-dichlorophenyl)-2-(dimethylamino)ethyl)-[1,1′-biphenyl]-4-sulfonamide;
(S)—N-(1-(4-chlorophenyl)-2-(dimethylamino)ethyl)-4-phenoxybenzenesulfonamide;
(S)—N-(2-(dimethylamino)-1-(3-(trifluoromethyl)phenyl)ethyl)-4-phenoxybenzenesulfonamide;
(S)-3-chloro-N-(1-(3,4-dichlorophenyl)-2-(dimethylamino)ethyl)-4-(trifluoromethoxy)benzenesulfonamide;
(S)-4-(cyclopentyloxy)-N-(1-(3,4-dichlorophenyl)-2-(dimethylamino)ethyl)benzenesulfonamide;
(S)—N-(1-(3,4-dichlorophenyl)-2-(dimethylamino)ethyl)-4-propylbenzenesulfonamide;
(S)—N-(1-(3,4-dichlorophenyl)-2-(4-methylpiperazin-1-yl)ethyl)-4-(trifluoromethoxy)benzenesulfonamide;
(S)—N-(1-(3,4-dichlorophenyl)-2-(dimethylamino)ethyl)-[1,1′-biphenyl]-3-sulfonamide;
(S)-3-chloro-N-(1-(4-chlorophenyl)-2-(dimethylamino)ethyl)-4-(trifluoromethoxy)benzenesulfonamide;
(S)-3-chloro-N-(2-(dimethylamino)-1-(3-(trifluoromethyl)phenyl)ethyl)-4-(trifluoromethoxy)benzenesulfonamide;
(S)—N-(1-(3-bromo-5-fluorophenyl)-2-cyclopropylethyl)-4-(trifluoromethoxy)benzenesulfonamide;
(S)—N-((1-(2,4-difluorophenyl)-2,2-dimethylcyclopropyl)methyl)-4-(trifluoromethoxy)benzenesulfonamide;
(S)—N-(1-(3,4-dichlorophenyl)-2-(dimethylamino)ethyl)-4-(trifluoromethoxy)benzenesulfonamide;
(S)—N-(2-(dimethylamino)-1-(3-(trifluoromethyl)phenyl)ethyl)-4-(4-(trifluoromethyl)phenoxy)benzenesulfonamide;
(S)—N-(2-(isopropyl(methyl)amino)benzyl)-4-(trifluoromethoxy)benzenesulfonamide;
(S)-4-(trifluoromethoxy)-N-(1-(4-(trifluoromethyl)phenyl)pent-4-en-1-yl)benzenesulfonamide;
N—((S)-((1S,2S)-2-methylcyclopropyl)(phenyl)methyl)-4-(trifluoromethoxy)benzenesulfonamide;
N—((S)-((1S,2R)-2-methylcyclopropyl)(phenyl)methyl)-4-(trifluoromethoxy)benzenesulfonamide;
(S)—N-(1-(3,4-dichlorophenyl)-2-(dimethylamino)ethyl)-3-(trifluoromethoxy)benzenesulfonamide;
(S)-2-amino-N-(1-(3,4-dichlorophenyl)-2-(dimethylamino)ethyl)-4-(trifluoromethoxy)benzenesulfonamide;
(S)—N-(1-(3,4-dichlorophenyl)-2-(dimethylamino)ethyl)naphthalene-1-sulfonamide;
(S)-2-chloro-N-(1-(3,4-dichlorophenyl)-2-(dimethylamino)ethyl)-4-(trifluoromethoxy)benzenesulfonamide;
(S)—N-(1-(3,4-dichlorophenyl)-2-(dimethylamino)ethyl)naphthalene-2-sulfonamide;
(S)-3,4-dichloro-N-(1-(3,4-dichlorophenyl)-2-(dimethylamino)ethyl)benzenesulfonamide;
N-(((2S,3S)-2-(4-methoxyphenyl)-1-methylpyrrolidin-3-yl)methyl)-4-(trifluoromethoxy)benzenesulfonamide;
N-(((2S,3R)-2-(4-methoxyphenyl)-1-methylpyrrolidin-3-yl)methyl)-4-(trifluoromethoxy)benzenesulfonamide;
(S)—N-(1-hydroxy-3-(m-tolyl)propan-2-yl)-4-(trifluoromethoxy)benzenesulfonamide;
(S)—N-(1-(3,4-dichlorophenyl)-2-(dimethylamino)ethyl)-2-phenylethane-1-sulfonamide;
(S)—N-(1-(3,4-dichlorophenyl)-2-(pyrrolidin-1-yl)ethyl)-4-(trifluoromethoxy)benzenesulfonamide;
(S)—N-(1-(3,4-dichlorophenyl)-2-(dimethylamino)ethyl)-N′-methyl-4-(trifluoromethoxy)benzenesulfonimidamide;
(S)—N-(1-(3,4-dichlorophenyl)-2-(dimethylamino)ethyl)-2,2-difluorobenzo[d][1,3]dioxole-5-sulfonamide;
(S)-ethyl 3-((4-(trifluoromethoxy)phenyl)sulfonamido)-3-(3-(trifluoromethyl)phenyl)propanoate;
(S)—N-(1-(1-(4-bromophenyl)cyclopropyl)ethyl)-4-(trifluoromethoxy)benzenesulfonamide;
(S)-4-methyl-N-(thiazol-2-yl)-2-((4-(trifluoromethoxy)phenyl)sulfonamido)pentanamide;
(S)—N-(1-(3,4-dichlorophenyl)-2-(dimethylamino)ethyl)-4-isopropylbenzenesulfonamide;
(S)—N-(1-(3,4-dichlorophenyl)-2-(dimethylamino)ethyl)-4-(trifluoromethyl)benzenesulfonamide;
(S)—N-(4,4,4-trifluoro-1-(2,4,5-trifluorophenyl)butan-2-yl)-4-(trifluoromethoxy)benzenesulfonamide;
(S)—N-((6-benzyl-6-azaspiro[3.4]octan-8-yl)methyl)-4-(trifluoromethoxy)benzenesulfonamide;
(S)-4-chloro-N-(1-(3,4-dichlorophenyl)-2-(dimethylamino)ethyl)benzenesulfonamide;
(S)—N-(1-(3-cyanophenyl)-3-methylbutyl)-4-(trifluoromethoxy)benzenesulfonamide;
(S)—N-(cyclohexyl(3-(trifluoromethyl)phenyl)methyl)-4-(trifluoromethoxy)benzenesulfonamide;
(S,E)-N-(1-(3,4-dichlorophenyl)-2-(dimethylamino)ethyl)-2-phenylethene-1-sulfonamide;
(S)—N-(2-(2-hydroxypropoxy)benzyl)-4-(trifluoromethoxy)benzenesulfonamide;
(S)-3-amino-N-(1-(3,4-dichlorophenyl)-2-(dimethylamino)ethyl)-4-(trifluoromethoxy)benzenesulfonamide;
N-((1S)-(4-fluorophenyl)(tetrahydrofuran-2-yl)methyl)-4-(trifluoromethoxy)benzenesulfonamide;
(S)—N-(1-(4-chlorophenyl)-2-(piperidin-1-yl)ethyl)-4-(trifluoromethoxy)benzenesulfonamide;
N—((S)-1-((S)-1-methylpiperidin-2-yl)propan-2-yl)-4-(trifluoromethoxy)benzenesulfonamide;
N—((S)-1-((R)-1-methylpiperidin-2-yl)propan-2-yl)-4-(trifluoromethoxy)benzenesulfonamide;
(S)—N-(1-(3,4-dichlorophenyl)-2-(dimethylamino)ethyl)-4-ethylbenzenesulfonamide;
(S)—N-(1-(2,5-difluorophenyl)propyl)-4-(trifluoromethoxy)benzenesulfonamide;
methyl (S)-3-(3,5-dichlorophenyl)-3-((4-(trifluoromethoxy)phenyl)sulfonamido)propanoate;
(S)—N-((3-chlorophenyl)(cyclopentyl)methyl)-4-(trifluoromethoxy)benzenesulfonamide;
(S)—N-(1-(benzo[d][1,3]dioxol-5-yl)butyl)-4-(trifluoromethoxy)benzenesulfonamide;
methyl (S)-3-(3-fluoro-4-hydroxyphenyl)-2-((4-(trifluoromethoxy)phenyl)sulfonamido)propanoate;
methyl (S)-3-(3-bromo-4-methoxyphenyl)-3-((4-(trifluoromethoxy)phenyl)sulfonamido)propanoate;
tert-butyl (S)-2-((4-(trifluoromethoxy)phenyl)sulfonamido)hexanoate;
(S)—N-(cyclopropyl(4-fluoro-3-(trifluoromethyl)phenyl)methyl)-4-(trifluoromethoxy)benzenesulfonamide;
(S)—N-(1-(3,4-dichlorophenyl)-2-hydroxyethyl)-4-(trifluoromethoxy)benzenesulfonamide;
(S)—N-(1-(3,4-dichlorophenyl)-2-(dimethylamino)ethyl)-3-methylbenzenesulfonamide;
methyl (S)-2-(2-bromophenyl)-2-((4-(trifluoromethoxy)phenyl)sulfonamido)acetate;
tert-butyl (S)-4,4-difluoro-2-(((4-(trifluoromethoxy)phenyl)sulfonamido)methyl)pyrrolidine-1-carboxylate;
(S)—N-(1-(3-chlorophenyl)-2-(dimethylamino)ethyl)-4-(trifluoromethoxy)benzenesulfonamide;
(S)—N-(2-morpholino-2-(4-(trifluoromethyl)phenyl)ethyl)-4-(trifluoromethoxy)benzenesulfonamide;
(S)—N-(2-(dimethylamino)-1-(p-tolyl)ethyl)-4-(trifluoromethoxy)benzenesulfonamide;
(S)—N-(1-(3,4-dichlorophenyl)-2-(dimethylamino)ethyl)-4-(difluoromethoxy)benzenesulfonamide;
(S)—N-(2-(furan-2-yl)-2-(4-methylpiperidin-1-yl)ethyl)-4-(trifluoromethoxy)benzenesulfonamide;
ethyl (S)-3-(2-chlorophenyl)-2-((4-(trifluoromethoxy)phenyl)sulfonamido)propanoate;
(S)—N-(2-(tert-butoxy)benzyl)-4-(trifluoromethoxy)benzenesulfonamide;
(S)-3-chloro-N-(1-(3,4-dichlorophenyl)-2-(dimethylamino)ethyl)benzenesulfonamide;
(S)—N-(2-(dimethylamino)-1-(3-(trifluoromethyl)phenyl)ethyl)-4-(trifluoromethoxy)benzenesulfonamide;
(S,S)—N-(1-(3,4-dichlorophenyl)-2-(dimethylamino)ethyl)-4-(trifluoromethoxy)benzenesulfonimidamide;
(S,R)—N-(1-(3,4-dichlorophenyl)-2-(dimethylamino)ethyl)-4-(trifluoromethoxy)benzenesulfonimidamide;
(S)—N-(1-(5-fluoro-2-(4-hydroxypiperidin-1-yl)phenyl)ethyl)-4-(trifluoromethoxy)benzenesulfonamide;
(S)—N-(2-(dimethylamino)-1-(m-tolyl)ethyl)-4-(trifluoromethoxy)benzenesulfonamide;
ethyl (S)-3-(3-bromophenyl)-3-((4-(trifluoromethoxy)phenyl)sulfonamido)propanoate;
(S)—N-(1-(3,4-dichlorophenyl)-2-(dimethylamino)ethyl)-2-methylbenzenesulfonamide;
(S)-4-chloro-N-(1-(3,4-dichlorophenyl)-2-(dimethylamino)ethyl)-3-fluorobenzenesulfonamide;
ethyl (S)-3-(3-chlorophenyl)-2-((4-(trifluoromethoxy)phenyl)sulfonamido)propanoate;
(S)—N-(1-(2-bromo-4-fluorophenyl)-2-cyclopropylethyl)-4-(trifluoromethoxy)benzenesulfonamide;
ethyl (S)-2-(3-bromo-4-methoxyphenyl)-2-((4-(trifluoromethoxy)phenyl)sulfonamido)acetate;
methyl (S)-3-(2-hydroxyphenyl)-2-((4-(trifluoromethoxy)phenyl)sulfonamido)propanoate;
(S)—N-(1-(3,4-dichlorophenyl)-3-methoxypropyl)-4-(trifluoromethoxy)benzenesulfonamide;
(S)—N-(1-(3,4-dichlorophenyl)-2-(dimethylamino)ethyl)-4-ethoxybenzenesulfonamide;
(S)—N-(1-(4-chlorophenyl)-2-(pyrrolidin-1-yl)ethyl)-4-(trifluoromethoxy)benzenesulfonamide;
(S)—N-(1-(3,4-dichlorophenyl)-2-(dimethylamino)ethyl)-4-methylbenzenesulfonamide;
(S)—N′-(1-(3,4-dichlorophenyl)-2-(dimethylamino)ethyl)-N-methyl-4-(trifluoromethoxy)benzenesulfonimidamide;
(S)—N-(1-(3,4-dichlorophenyl)-2-(dimethylamino)ethyl)-3-(trifluoromethyl)benzenesulfonamide;
(S)—N-cyclohexyl-4-(N-(1-(3,4-dichlorophenyl)-2-(dimethylamino)ethyl)sulfamoyl)benzamide;
(S)—N-(1-(3,4-dichlorophenyl)-2-morpholinoethyl)-4-(trifluoromethoxy)benzenesulfonamide;
(S)—N-cycloheptyl-N-methyl-2-((4-(trifluoromethoxy)phenyl)sulfonamido)pentanamide;
(S)—N-(4,4,4-trifluoro-3-phenylbutyl)-4-(trifluoromethoxy)benzenesulfonamide;
(S)—N-(1-(5-ethylfuran-2-yl)-2,2-dimethylpropyl)-4-(trifluoromethoxy)benzenesulfonamide;
(S)—N-(2-(4-ethylphenyl)-2-(pyrrolidin-1-yl)ethyl)-4-(trifluoromethoxy)benzenesulfonamide;
(S)—N-(1-(4-chlorophenyl)-2-(dimethylamino)ethyl)-4-(trifluoromethoxy)benzenesulfonamide;
methyl (S)-3-(3,4-dichlorophenyl)-2-((4-(trifluoromethoxy)phenyl)sulfonamido)propanoate;
(S)—N-(1-(3,4-dichlorophenyl)-2-(dimethylamino)ethyl)cyclohexanesulfonamide;
ethyl (S)-3-(2,5-dichlorothiophen-3-yl)-2-((4-(trifluoromethoxy)phenyl)sulfonamido)propanoate;
tert-butyl ((4-(trifluoromethoxy)phenyl)sulfonyl)-L-isoleucinate;
tert-butyl ((4-(trifluoromethoxy)phenyl)sulfonyl)-L-alloisoleucinate
(S)-4-chloro-N-(1-(3,4-dichlorophenyl)-2-(dimethylamino)ethyl)-2-fluorobenzenesulfonamide;
(S)—N-(1-(3-bromophenyl)-4,4,4-trifluorobutan-2-yl)-4-(trifluoromethoxy)benzenesulfonamide;
(S)—N-(2-(dimethylamino)-1-(o-tolyl)ethyl)-4-(trifluoromethoxy)benzenesulfonamide;
methyl (S)-3-cyclohexyl-3-((4-(trifluoromethoxy)phenyl)sulfonamido)propanoate;
methyl (S)-4-phenyl-3-((4-(trifluoromethoxy)phenyl)sulfonamido)butanoate;
(S)—N-(1-(3,4-dichlorophenyl)-2-(dimethylamino)ethyl)-4-fluorobenzenesulfonamide;
(S)-3-(4-isopropylphenyl)-3-((4-(trifluoromethoxy)phenyl)sulfonamido)propanoic acid;
methyl (S)-2-phenyl-2-((4-(trifluoromethoxy)phenyl)sulfonamido)butanoate;
(S)—N-(1-(3,4-dichlorophenyl)-2-(dimethylamino)ethyl)-3-fluorobenzenesulfonamide;
ethyl (S)-4-methyl-2-((4-(trifluoromethoxy)phenyl)sulfonamido)hexanoate;
(S)—N-(3-(1-cyanopropoxy)benzyl)-4-(trifluoromethoxy)benzenesulfonamide;
ethyl (S)-3-(4-methoxyphenyl)-2-((4-(trifluoromethoxy)phenyl)sulfonamido)propanoate;
ethyl (S)-3-(4-isopropoxy-3-methoxyphenyl)-3-((4-(trifluoromethoxy)phenyl)sulfonamido)propanoate;
(S)—N-((4-chlorophenyl)(cyclopentyl)methyl)-4-(trifluoromethoxy)benzenesulfonamide;
ethyl (S)-3-(4-ethoxyphenyl)-3-((4-(trifluoromethoxy)phenyl)sulfonamido)propanoate;
(S)—N-(1-(3,4-dichlorophenyl)-2-(dimethylamino)ethyl)-3,5-difluorobenzenesulfonamide;
(S)—N-(1-(2-methoxy-5-methylphenyl)-3-methylbutan-2-yl)-4-(trifluoromethoxy)benzenesulfonamide;
N-(2-(((1S,2S)-2-methylcyclohexyl)oxy)ethyl)-4-(trifluoromethoxy)benzenesulfonamide;
N-(2-(((1S,2R)-2-methylcyclohexyl)oxy)ethyl)-4-(trifluoromethoxy)benzenesulfonamide;
N-(((1S,2S)-2-phenylcyclohexyl)methyl)-4-(trifluoromethoxy)benzenesulfonamide;
N-(((1S,2R)-2-phenylcyclohexyl)methyl)-4-(trifluoromethoxy)benzenesulfonamide;
N-(((1S,3S)-1-(dimethylamino)-3-methylcyclohexyl)methyl)-4-(trifluoromethoxy)benzenesulfonamide;
N-(((1S,3R)-1-(dimethylamino)-3-methylcyclohexyl)methyl)-4-(trifluoromethoxy)benzenesulfonamide;
N-((1S,2S)-2-(methylsulfonyl)-1-(p-tolyl)propyl)-4-(trifluoromethoxy)benzenesulfonamide;
N-((1S,2R)-2-(methylsulfonyl)-1-(p-tolyl)propyl)-4-(trifluoromethoxy)benzenesulfonamide;
N-((1S,2S)-1-(2-chlorophenyl)-1-methoxypropan-2-yl)-4-(trifluoromethoxy)benzenesulfonamide;
N-((1S,2R)-1-(2-chlorophenyl)-1-methoxypropan-2-yl)-4-(trifluoromethoxy)benzenesulfonamide;
ethyl (S)-3-((S)-tetrahydrofuran-3-yl)-2-((4-(trifluoromethoxy)phenyl)sulfonamido)propanoate;
ethyl (S)-3-((R)-tetrahydrofuran-3-yl)-2-((4-(trifluoromethoxy)phenyl)sulfonamido)propanoate;
ethyl (S)-3-((S)-cyclohex-3-en-1-yl)-2-((4-(trifluoromethoxy)phenyl)sulfonamido)propanoate;
ethyl (S)-3-((R)-cyclohex-3-en-1-yl)-2-((4-(trifluoromethoxy)phenyl)sulfonamido)propanoate;
N—((S)-(4-fluorophenyl)((S)-tetrahydrofuran-2-yl)methyl)-4-(trifluoromethoxy)benzenesulfonamide;
N—((S)-(4-fluorophenyl)((R)-tetrahydrofuran-2-yl)methyl)-4-(trifluoromethoxy)benzenesulfonamide;
(S)-4-cyclopropoxy-N-(1-(3,4-dichlorophenyl)-2-(dimethylamino)ethyl)benzenesulfonamide;
(S)—N-(1-(3-chloro-4-fluorophenyl)-2-(dimethylamino)ethyl)-4-(trifluoromethoxy)benzenesulfonamide;
(S)—N-(1-(4-chloro-3-fluorophenyl)-2-(dimethylamino)ethyl)-4-(trifluoromethoxy)benzenesulfonamide;
(S)-4-(N-(1-(3,4-dichlorophenyl)-2-(dimethylamino)ethyl)sulfamoyl)-N-phenylbenzamide;
(S)—N-(1-(3,4-dichlorophenyl)-2-(dimethylamino)ethyl)-4-(phenylamino)benzenesulfonamide;
(S)—N-(1-(4-chlorophenyl)-2-(dimethylamino)ethyl)-4-(4-(trifluoromethyl)phenoxy)benzenesulfonamide;
(S)—N-(2-(dimethylamino)-1-(3-(trifluoromethyl)phenyl)ethyl)-3-nitro-4-(trifluoromethoxy)benzenesulfonamide;
(S)—N-(1-(4-chlorophenyl)-2-(dimethylamino)ethyl)-3-nitro-4-(trifluoromethoxy)benzenesulfonamide;
(S)-4-(cyclopropylmethoxy)-N-(1-(3,4-dichlorophenyl)-2-(dimethylamino)ethyl)benzenesulfonamide;
(S)—N-(1-(3,4-dichlorophenyl)-2-(dimethylamino)ethyl)-4-isopropoxybenzenesulfonamide;
(S)-4-(cyclohexyloxy)-N-(1-(3,4-dichlorophenyl)-2-(dimethylamino)ethyl)benzenesulfonamide;
(S)—N-(1-(3,4-dichlorophenyl)-2-(dimethylamino)ethyl)-4-(pyridin-2-yloxy)benzenesulfonamide;
(S)-4-butyl-N-(1-(3,4-dichlorophenyl)-2-(dimethylamino)ethyl)benzenesulfonamide;
(S)—N-(1-(3,4-dichlorophenyl)-2-(dimethylamino)ethyl)-4-(3,6-dihydro-2H-pyran-4-yl)benzenesulfonamide;
(S)—N-(2-(dimethylamino)-1-(4-(trifluoromethyl)phenyl)ethyl)-4-(trifluoromethoxy)benzenesulfonamide;
(S)—N-(1-(3,4-dichlorophenyl)-2-(dimethylamino)ethyl)-4-(3-fluorophenoxy)benzenesulfonamide;
(S)—N-(1-(3,4-dichlorophenyl)-2-(dimethylamino)ethyl)-4-(4-fluorophenoxy)benzenesulfonamide;
(S)-methyl 5-(N-(1-(3,4-dichlorophenyl)-2-(dimethylamino)ethyl)sulfamoyl)-2-(trifluoromethoxy)benzoate;
(S)—N-(1-(3,4-dichlorophenyl)-2-(4-methylpiperazin-1-yl)ethyl)-4-(4-(trifluoromethyl)phenoxy)benzenesulfonamide;
(S)—N-(1-(4-chlorophenyl)-2-(4-methylpiperazin-1-yl)ethyl)-4-phenoxybenzenesulfonamide;
(S)—N-(1-(4-chlorophenyl)-2-(4-methylpiperazin-1-yl)ethyl)-4-(4-(trifluoromethyl)phenoxy)benzenesulfonamide;
(S)—N-(1-(3,4-dichlorophenyl)-2-(4-methylpiperazin-1-yl)ethyl)-4-phenoxybenzenesulfonamide;
(S)—N-(2-(dimethylamino)-1-(3-(trifluoromethyl)phenyl)ethyl)-4-(4-(trifluoromethyl)phenoxy)benzenesulfonamide;
(S)—N-(1-(3,4-dichlorophenyl)-2-(dimethylamino)ethyl)-6-(trifluoromethoxy)pyridine-3-sulfonamide;
(S)—N-(1-(3,4-dichlorophenyl)-2-(dimethylamino)ethyl)-3-nitro-4-(trifluoromethoxy)benzenesulfonamide;
(S)—N-(1-(3,4-dichlorophenyl)-2-(dimethylamino)ethyl)-4-(pyridin-2-yl)benzenesulfonamide;
(S)—N-(1-(3,4-dichlorophenyl)-2-(dimethylamino)ethyl)-4-nitro-3-(trifluoromethoxy)benzenesulfonamide;
(S)—N-(2-amino-1-(3,4-dichlorophenyl)ethyl)-4-(trifluoromethoxy)benzenesulfonamide hydrochloride;
(S)-tert-butyl (2-(3,4-dichlorophenyl)-2-((4-(trifluoromethoxy)phenyl)sulfonamido)ethyl)carbamate;
(S)—N-((2,2-dimethyl-1-phenylcyclopropyl)methyl)-4-(trifluoromethoxy)benzenesulfonamide;
(S)—N-(1-(3,4-dichlorophenyl)-2-(dimethylamino)ethyl)-2-nitro-3-(trifluoromethoxy)benzenesulfonamide;
4-(trifluoromethoxy)-N-((1S,3S)-3,4,4-trimethylcyclohexyl)benzenesulfonamide;
4-(trifluoromethoxy)-N-((1S,3R)-3,4,4-trimethylcyclohexyl)benzenesulfonamide;
(S)—N-(1-(3,4-dichlorophenyl)-2-(dimethylamino)ethyl)-4-(2-(trifluoromethyl)phenoxy)benzenesulfonamide;
(S)—N-(1-(3,4-dichlorophenyl)-2-(dimethylamino)ethyl)-4-(tetrahydro-2H-pyran-2-yl)benzenesulfonamide;
(S)-3-cyano-N-(1-(3,4-dichlorophenyl)-2-(dimethylamino)ethyl)-4-(trifluoromethoxy)benzenesulfonamide;
(S)—N-(1-(3,4-dichlorophenyl)-2-(4-propoxypiperidin-1-yl)ethyl)-4-(trifluoromethoxy)benzenesulfonamide;
(S)—N-(1-(3,4-dichlorophenyl)-2-(methylamino)ethyl)-4-(trifluoromethoxy)benzenesulfonamide;
(S)—N-(1-(3,4-dichlorophenyl)-3-(dimethylamino)propan-2-yl)-4-(trifluoromethoxy)benzenesulfonamide;
(S)-4-cyclobutoxy-N-(1-(3,4-dichlorophenyl)-2-(dimethylamino)ethyl)benzenesulfonamide;
(S)-3-(3,4-dichlorophenyl)-N,N-dimethyl-2-((4-(trifluoromethoxy)phenyl)sulfonamido)propenamide;
(S)—N-(1-(3,4-dichlorophenyl)-2-(dimethylamino)ethyl)-N-methyl-4-(trifluoromethoxy)benzenesulfonamide;
(S)-4-(cyclopentylamino)-N-(1-(3,4-dichlorophenyl)-2-(dimethylamino)ethyl)benzenesulfonamide;
(S)-4-(cyclobutylamino)-N-(1-(3,4-dichlorophenyl)-2-(dimethylamino)ethyl)benzenesulfonamide;
(S)—N-((3-(3,4-dichlorophenyl)pyrrolidin-3-yl)methyl)-4-(trifluoromethoxy)benzenesulfonamide;
(S)-2-(3,4-dichlorophenyl)-N,N-dimethyl-2-((4-(trifluoromethoxy)phenyl)sulfonamido)acetamide;
(S)—N-(1-(3,4-dichlorophenyl)-2-((4-methoxybenzyl)amino)ethyl)-4-(trifluoromethoxy)benzenesulfonamide;
(S)—N-(1-(4-chlorophenyl)-2-(piperazin-1-yl)ethyl)-4-(trifluoromethoxy)benzenesulfonamide;
(S)—N-(1-(4-chloro-3-fluorophenyl)-2-(piperazin-1-yl)ethyl)-4-(trifluoromethoxy)benzenesulfonamide;
(S)—N-(1-(4-chlorophenyl)-2-(piperazin-1-yl)ethyl)-6-(trifluoromethoxy)pyridine-3-sulfonamide;
N—((S)-2-((1R,4R)-2,5-diazabicyclo[2.2.1]heptan-2-yl)-1-(3,4-dichlorophenyl)ethyl)-4-(trifluoromethoxy)benzenesulfonamide;
N—((S)-2-((1S,4S)-2,5-diazabicyclo[2.2.1]heptan-2-yl)-1-(3,4-dichlorophenyl)ethyl)-4-(trifluoromethoxy)benzenesulfonamide;
(S)—N-((2-phenylpyrrolidin-2-yl)methyl)-4-(trifluoromethoxy)benzenesulfonamide;
(S)—N-(1-(4-fluorophenyl)-2-(piperazin-1-yl)ethyl)-4-(trifluoromethoxy)benzenesulfonamide;
(S)—N-(2-(4-fluorophenyl)-2-(piperazin-1-yl)ethyl)-4-(trifluoromethoxy)benzenesulfonamide;
(S)—N-(1-(3,4-dichlorophenyl)-2-(dimethylamino)ethyl)-3-nitro-4-(trifluoromethyl)benzenesulfonamide;
(S)—N-(1-(3,4-dichlorophenyl)-2-(dimethylamino)ethyl)-4-propoxybenzenesulfonamide;
(S)-4-(cyclohexylamino)-N-(1-(3,4-dichlorophenyl)-2-(dimethylamino)ethyl)benzenesulfonamide; and
(S)—N-(1-(3,4-dichlorophenyl)-2-(4-methylpiperazin-1-yl)ethyl)-4-(4-fluorophenoxy)benzenesulfonamide;
or a salt, solvate, isotopologue, or tautomer thereof.

In certain embodiments, the compound is selected from the group consisting of
N-(3,3,3-trifluoro-1-phenylpropyl)-4-(trifluoromethoxy)benzenesulfonamide;
(R)—N-(3,3,3-trifluoro-1-phenylpropyl)-4-(trifluoromethoxy)benzenesulfonamide;
(S)—N-(3,3,3-trifluoro-1-phenylpropyl)-4-(trifluoromethoxy)benzenesulfonamide;
N-(3-hydroxy-1-phenylpropyl)-4-(trifluoromethoxy)benzenesulfonamide;
(R)—N-(3-hydroxy-1-phenylpropyl)-4-(trifluoromethoxy)benzenesulfonamide;
(S)—N-(3-hydroxy-1-phenylpropyl)-4-(trifluoromethoxy)benzenesulfonamide;
N-(2-cyclopropyl-1-phenylethyl)-4-(trifluoromethoxy)benzenesulfonamide;
(R)—N-(2-cyclopropyl-1-phenylethyl)-4-(trifluoromethoxy)benzenesulfonamide;
(S)—N-(2-cyclopropyl-1-phenylethyl)-4-(trifluoromethoxy)benzenesulfonamide;
methyl 2-methyl-3-phenyl-3-((4-(trifluoromethoxy)phenyl)sulfonamido)propanoate;
methyl (2R,3R)-2-methyl-3-phenyl-3-((4-(trifluoromethoxy)phenyl)sulfonamido)propanoate;
methyl (2R,3S)-2-methyl-3-phenyl-3-((4-(trifluoromethoxy)phenyl)sulfonamido)propanoate;
methyl (2S,3S)-2-methyl-3-phenyl-3-((4-(trifluoromethoxy)phenyl)sulfonamido)propanoate;
methyl (2S,3R)-2-methyl-3-phenyl-3-((4-(trifluoromethoxy)phenyl)sulfonamido)propanoate;
ethyl 3-(p-tolyl)-3-((4-(trifluoromethoxy)phenyl)sulfonamido)propanoate;
ethyl (R)-3-(p-tolyl)-3-((4-(trifluoromethoxy)phenyl)sulfonamido)propanoate;
ethyl (S)-3-(p-tolyl)-3-((4-(trifluoromethoxy)phenyl)sulfonamido)propanoate;
N-(4,4,4-trifluoro-3-hydroxy-3-(trifluoromethyl)butyl)-4-(trifluoromethoxy)benzenesulfonamide;
N-((1-(2-methoxyethyl)cyclohexyl)methyl)-4-(trifluoromethoxy)benzenesulfonamide;
N-(2-(diisobutylamino)ethyl)-4-(trifluoromethoxy)benzenesulfonamide;
N-(3-(benzyl(butyl)amino)propyl)-4-(trifluoromethoxy)benzenesulfonamide;
N-(1-(2,6-dichlorophenyl)propan-2-yl)-4-(trifluoromethoxy)benzenesulfonamide;
(R)—N-(1-(2,6-dichlorophenyl)propan-2-yl)-4-(trifluoromethoxy)benzenesulfonamide;
(S)—N-(1-(2,6-dichlorophenyl)propan-2-yl)-4-(trifluoromethoxy)benzenesulfonamide;
tert-butyl 2,2-dimethyl-6-(((4-(trifluoromethoxy)phenyl)sulfonamido)methyl)morpholine-4-carboxylate;
(R)-tert-butyl 2,2-dimethyl-6-(((4-(trifluoromethoxy)phenyl)sulfonamido)methyl)morpholine-4-carboxylate;
(S)-tert-butyl 2,2-dimethyl-6-(((4-(trifluoromethoxy)phenyl)sulfonamido)methyl)morpholine-4-carboxylate;
N-(3-((furan-2-ylmethyl)((tetrahydrofuran-2-yl)methyl)amino)propyl)-4-(trifluoromethoxy)benzenesulfonamide;
(R)—N-(3-((furan-2-ylmethyl)((tetrahydrofuran-2-yl)methyl)amino)propyl)-4-(trifluoromethoxy)benzenesulfonamide;
(S)—N-(3-((furan-2-ylmethyl)((tetrahydrofuran-2-yl)methyl)amino)propyl)-4-(trifluoromethoxy)benzenesulfonamide;
N-((1-(2-(benzyloxy)ethyl)cyclopropyl)methyl)-4-(trifluoromethoxy)benzenesulfonamide;
N-(3-(2-(benzyloxy)ethoxy)propyl)-4-(trifluoromethoxy)benzenesulfonamide;
N-(1-(1-methylpiperidin-2-yl)propan-2-yl)-4-(trifluoromethoxy)benzenesulfonamide;
N-(2-((2-methylcyclohexyl)oxy)ethyl)-4-(trifluoromethoxy)benzenesulfonamide;
N-((1-((dimethylamino)methyl)cyclopropyl)methyl)-4-(trifluoromethoxy)benzenesulfonamide;
N-((1-(dimethylamino)-3-methylcyclohexyl)methyl)-4-(trifluoromethoxy)benzenesulfonamide;
N-(2-(2,2-dioxidobenzo[c][1,2,5]thiadiazol-1(3H)-yl)ethyl)-4-(trifluoromethoxy)benzenesulfonamide;
N-((3-methylcyclohexyl)(o-tolyl)methyl)-4-(trifluoromethoxy)benzenesulfonamide;
(R)—N-((3-methylcyclohexyl)(o-tolyl)methyl)-4-(trifluoromethoxy)benzenesulfonamide;
(S)—N-((3-methylcyclohexyl)(o-tolyl)methyl)-4-(trifluoromethoxy)benzenesulfonamide;
N-((2-methylcyclopropyl)(phenyl)methyl)-4-(trifluoromethoxy)benzenesulfonamide;
(R)—N-((2-methylcyclopropyl)(phenyl)methyl)-4-(trifluoromethoxy)benzenesulfonamide;
(S)—N-((2-methylcyclopropyl)(phenyl)methyl)-4-(trifluoromethoxy)benzenesulfonamide;
N-(2-(methylsulfonyl)-1-(p-tolyl)propyl)-4-(trifluoromethoxy)benzenesulfonamide;
tert-butyl (1-phenyl-2-((4-(trifluoromethoxy)phenyl)sulfonamido)propyl)carbamate;
tert-butyl ((1R,2R)-1-phenyl-2-((4-(trifluoromethoxy)phenyl)sulfonamido)propyl)carbamate;
tert-butyl ((1R,2S)-1-phenyl-2-((4-(trifluoromethoxy)phenyl)sulfonamido)propyl)carbamate;
tert-butyl ((1S,2S)-1-phenyl-2-((4-(trifluoromethoxy)phenyl)sulfonamido)propyl)carbamate;
tert-butyl ((1S,2R)-1-phenyl-2-((4-(trifluoromethoxy)phenyl)sulfonamido)propyl)carbamate;
tert-butyl (2-phenyl-2-((4-(trifluoromethoxy)phenyl)sulfonamido)ethyl)carbamate;
tert-butyl (R)-(2-phenyl-2-((4-(trifluoromethoxy)phenyl)sulfonamido)ethyl)carbamate;
tert-butyl (S)-(2-phenyl-2-((4-(trifluoromethoxy)phenyl)sulfonamido)ethyl)carbamate;
N-(2-amino-1-phenylethyl)-4-(trifluoromethoxy)benzenesulfonamide;
(R)—N-(2-amino-1-phenylethyl)-4-(trifluoromethoxy)benzenesulfonamide;
(S)—N-(2-amino-1-phenylethyl)-4-(trifluoromethoxy)benzenesulfonamide;
N-(1-phenyl-2-(4-propoxypiperidin-1-yl)ethyl)-4-(trifluoromethoxy)benzenesulfonamide;
(R)—N-(1-phenyl-2-(4-propoxypiperidin-1-yl)ethyl)-4-(trifluoromethoxy)benzenesulfonamide;
(S)—N-(1-phenyl-2-(4-propoxypiperidin-1-yl)ethyl)-4-(trifluoromethoxy)benzenesulfonamide;
1-methyl-N-(2-phenyl-2-((4-(trifluoromethoxy)phenyl)sulfonamido)ethyl)-1H-pyrrole-3-sulfonamide;
(R)-1-methyl-N-(2-phenyl-2-((4-(trifluoromethoxy)phenyl)sulfonamido)ethyl)-1H-pyrrole-3-sulfonamide;
(S)-1-methyl-N-(2-phenyl-2-((4-(trifluoromethoxy)phenyl)sulfonamido)ethyl)-1H-pyrrole-3-sulfonamide;
N-(1-(dimethylamino)propan-2-yl)-3-nitro-4-(trifluoromethoxy)benzenesulfonamide;
(R)—N-(1-(dimethylamino)propan-2-yl)-3-nitro-4-(trifluoromethoxy)benzenesulfonamide; and
(S)—N-(1-(dimethylamino)propan-2-yl)-3-nitro-4-(trifluoromethoxy)benzenesulfonamide;
or a salt, solvate, isotopologue, or tautomer thereof.

The compounds of the invention may possess one or more stereocenters, and each stereocenter may exist independently in either the (R) or (S) configuration. In certain embodiments, compounds described herein are present in optically active or racemic forms.

The compounds described herein encompass racemic, optically active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or mixtures thereof, or in the case where two or more chiral center are present, all diastereomers or mixtures thereof.

In certain embodiments, the compounds of the invention exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature (i.e. isotopologues). Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O ³²P, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed invention. The compounds of the invention may contain any of the substituents, or combinations of substituents, provided herein.

### Salts

The compounds described herein may form salts with acids or bases, and such salts are included in the present invention. The term “salts” embraces addition salts of free acids or bases that are useful within the methods of the invention. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, aryl-aliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the invention.

Suitable pharmaceutically acceptable base addition salts of compounds of the invention include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

### Synthesis

The present invention further provides methods of preparing the compounds of the present invention. Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field.

It is appreciated that where typical or preferred process conditions (i.e., reaction temperatures, times, mole ratios of reactants, solvents, pressures, and so forth) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high pressure liquid chromatograpy (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents that can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

In certain embodiment, the compounds synthesized using the methods described herein may contain one or more chiral carbon atoms, giving rise to two or more isomers. The absolute stereochemistry may be depicted using wedge bonds (bold or parallel lines). In certain embodiments, the product formed in any of the reactions described may be a racemate. If a racemate is formed, the isomers making up the racemate may be separated using any suitable method for chiral resolution known to a person skilled in the art. Suitable methods for chiral resolution include, but are not limited to, supercritical fluid chromatography (SFC), chiral HPCL, crystallization, derivatization, or any combination thereof.

In certain embodiments, separation of the isomers formed in one or more separate reactions may require forming a derivative prior to chiral resolution. A non-limiting example of derivatization is protecting one or more functional groups present in a compound using known protecting groups (such as esters, amides, carbamates, ethers, etc.), followed by separation of the isomers by a suitable method. The desired compound is finally obtained through removal of the protecting group.

The compounds of the present disclosure having the structure of formula (I) can be prepared as described herein.

In certain embodiments, compounds of formula (I), which are compounds of formula (Ia) and (Ib), wherein Z, L, R^{a}, and R¹ are defined within the scope of the present disclosure, are prepared from the corresponding sulfinamide 1-1. Sulfinamide 1-1 may first be converted to a sulfonimidoyl chloride via oxidative chlorination under suitable reaction conditions and subsequently condensed with an amine (1-2) under suitable conditions to afford the desired sulfonimidamide species (Ia) and (Ib) (Scheme 1). Suitable oxidative chlorination conditions include, but are not limited to, t-BuOCl in THF. Suitable conditions for condensation of a sulfonimidoyl chloride with an amine include, but are not limited to, DIPEA in THF. Tautomeric sulfonimidamide species (Ia) and (Ib) may be separated chromatographically using methods known to one of ordinary skill in the art.

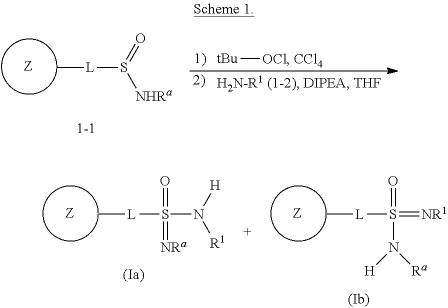

In certain embodiments, a compound of formula (I), which is a compound of formula (Ic), wherein Z, L, and R¹ are defined within the scope of the present disclosure, is prepared from the corresponding sulfonyl chloride 2-1 and an amine 2-2 in the presence of a suitable base and suitable solvent (Scheme 2). A suitable base for the sulfonylation is Et₃N. A suitable solvent for the sulfonylation is CH₂Cl₂.

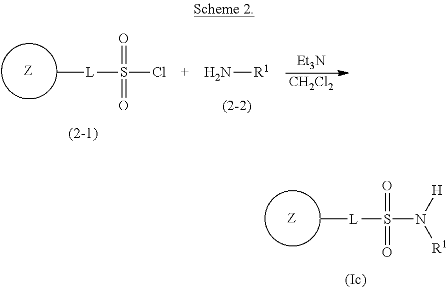

In certain embodiments, each of the sulfonyl chloride 2-1 and amine 2-2 compounds are commercially available. In other embodiments, at least one of the sulfonyl halide 2-1 and amine 2-2 compounds is prepared using synthetic methods known to one of ordinary skill in the art.

In certain embodiments, the amine of formula 2-2 is an amine of formula 3-5, wherein Ar is defined within the scope of the present disclosure and R′ is H, C₁-C₆ alkyl, or each R′ may combine with the atom to which they are bound to form a C₂-C₆ heterocycloalkyl. In general, the amine of formula 3-5 can be prepared according to the synthesis provided in Scheme 3.

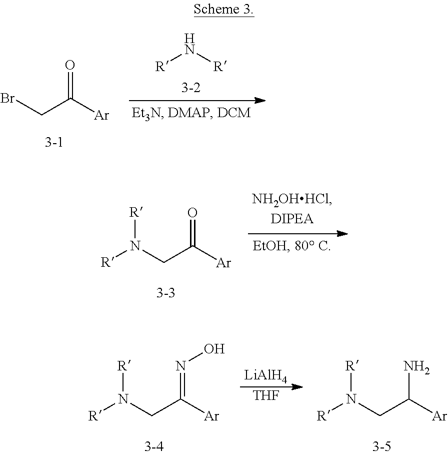

As illustrated in Scheme 3, α-halo arylketone 3-1 may undergo substitution with amine 3-2 in the presence of a suitable base and solvent, including but not limited to Et₃N and DMAP, and DCM, respectively, to afford α-amino arylketone 3-3. α-amino arylketone 3-3 may be condensed with hydroxylamine in the presence of a suitable base and solvent, including but not limited to DIPEA and EtOH, to afford oxime 3-4. Oxime 3-4 may be reduced with a suitable reducing agent and solvent, including but not limited to LiAlH₄ and THF to provide the desired amine 3-5.

In certain embodiments, the amine of formula 2-2 is an amine of formula 4-4, wherein Ar is defined within the scope of the present disclosure, R″ is H or C₁-C₃ alkyl, and G is a bond or —(CR″R″)ₘ—, wherein m is an integer selected from the group consisting of 1, 2, and 3. In general, the amine of formula 4-4 can be prepared according to the synthesis provided in Scheme 4.

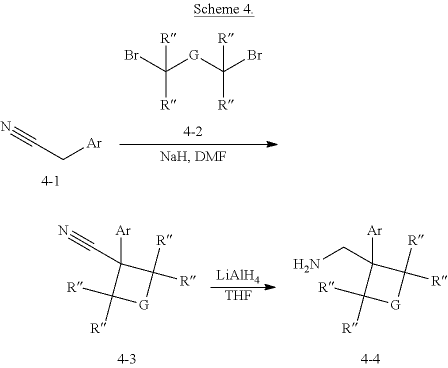

As illustrated in Scheme 4, α-aryl nitrile 4-1 may undergo two consecutive alkylation reactions with alkyl bromide 4-2 in the presence of a suitable base and solvent, including but not limited to NaH and DMF, to afford α-carbocyclic aryl nitrile species 4-3. Nitrile 4-3 may be reduced with a suitable reducing agent and solvent, including but not limited to LiAlH₄ and THF, to provide the desired amine 4-4.

In certain embodiments, compounds of formula (I) may be subjected to further synthetic manipulations, including but not limited to cross-coupling reactions (e.g. Suzuki cross-coupling and Buchwald-Hartwig amination), protecting group removal (e.g. Boc deprotection), and/or reduction/oxidation (e.g. reduction of an aryl nitro group to an aniline) to provide the biologically active compounds of the present disclosure.

### Methods

The present disclosure relates in part to a method of treating, preventing, and/or ameliorating a PP2A-related disease in a subject, the method comprising administering to a subject in need thereof a therapeutically effective amount of the compound of any one of the compounds of the present disclosure or a pharmaceutical composition of comprising any one of the compounds of the present disclosure and a pharmaceutically acceptable carrier.

In certain embodiments, the PP2A-related disease is at least one selected from the group consisting of cancer, diabetes, autoimmune disease, solid organ transplant rejection, graft vs host disease, chronic obstructive pulmonary disease (COPD), non-alcoholic fatty liver disease, abdominal aortic aneurysm, chronic liver disease, heart failure, neurodegenerative disease, and cardiac hypertrophy.

In certain embodiments, the subject is a mammal. In certain embodiments, the mammal is a human.

The present disclosure further relates in part to the use of a compound according to one or more embodiments disclosed herein, for example a compound of Formula (I), a salt, solvate, enantiomer, diastereomer, isotopologue, tautomer, or any mixture thereof, for use as a medicament for treating, preventing, and/or ameliorating a disease or condition in a patient.

The compounds according to one or more embodiments disclosed in this specification may be modulators of PP2A. The compounds described herein may exhibit anti-proliferative effects and may be useful as monotherapy in cancer treatment and/or in the treatment of other indications described in this specification. Additionally, they can be used in combination with other drugs to restore sensitivity to chemotherapy, targeted therapies, or immunotherapy where resistance has developed.

In certain embodiments, the disease or condition is ameliorated by modulation of PP2A. In certain embodiments, the disease or condition is at least one selected from the group consisting of cancer, diabetes, autoimmune disease, solid organ transplant rejection, graft vs host disease, chronic obstructive pulmonary disease (COPD), non-alcoholic fatty liver disease, abdominal aortic aneurysm, chronic liver disease, heart failure, neurodegenerative disease, and cardiac hypertrophy. In certain embodiments, the disease is cancer.

In certain embodiments, a patient in need of treatment of a disease is administered a therapeutically effective amount of the compound according to one or more embodiments disclosed in this specification, for example a compound of Formula (I), a solvate, enantiomer, diastereomer, isotopologue, tautomer, or a pharmaceutically acceptable salt thereof.

In certain embodiments, a method of treating cancer in a patient having a tumor that expresses PP2A comprises administering to the patient a therapeutically effective amount of a compound of Formula (I), a solvate, enantiomer, diastereomer, isotopologue, tautomer, or a pharmaceutically acceptable salt thereof.

In certain embodiments, a method is provided for treating a malignant solid tumor in a patient in need thereof, comprising administering an effective amount of a compound or pharmaceutical composition provided herein to the patient. In certain embodiments, the malignant solid tumor is a carcinoma. In certain embodiments, the malignant tumor is a lymphoma. In certain embodiments, the malignant solid tumor is a sarcoma.

In certain embodiments, the cancer is of the bladder, blood, bone, bone marrow, brain, breast, colon, esophagus, gastrointestine, gum, head, kidney, liver, lung, nasopharynx, neck, ovary, prostate, skin, stomach, testis, tongue, and/or uterus. In addition, the cancer may specifically be at least one of the following histological types, though it is not limited to these: neoplasm, malignant; carcinoma; carcinoma, undifferentiated; giant or spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant-cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast-cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast-cell leukemia; megakaryoblastic leukemia; myeloid sarcoma; and hairy cell leukemia.

In certain embodiments, the autoimmune disease is colitis, multiple sclerosis, arthritis, rheumatoid arthritis, osteoarthritis, juvenile arthritis, psoriatic arthritis, acute pancreatitis, chronic pancreatitis, diabetes, insulin-dependent diabetes mellitus (IDDM or type I diabetes), insulitis, inflammatory bowel disease, Crohn's disease, ulcerative colitis, autoimmune hemolytic syndromes, autoimmune hepatitis, autoimmune neuropathy, autoimmune ovarian failure, autoimmune orchitis, autoimmune thrombocytopenia, reactive arthritis, ankylosing spondylitis, silicone implant associated autoimmune disease, Sjogren's syndrome, systemic lupus erythematosus (SLE), vasculitis syndromes (e.g., giant-cell arteritis, Behcet's disease & Wegener's granulomatosis), vitiligo, secondary hematologic manifestation of autoimmune diseases (e.g., anemias), drug-induced autoimmunity, Hashimoto's thyroiditis, hypophysitis, idiopathic thrombocytic pupura, metal-induced autoimmunity, myasthenia gravis, pemphigus, autoimmune deafness (e.g., Meniere's disease), Goodpasture's syndrome, Graves' disease, HIV-related autoimmune syndromes and Gullain-Barre disease.

In certain embodiments, the neurodegenerative disease is Alzheimer's disease. In certain embodiments, the neurodegenerative disease is Parkinson's disease.

PP2A enzymes may be involved in the regulation of cell transcription, cell cycle, and viral transformation. Many viruses, including cytomegalovirus, parainfluenza, DNA tumor viruses, and HIV-1, utilize different approaches to exploit PP2A in order to modify, control, or inactivate cellular activities of the host. Therefore, the compounds according to one or more embodiments disclosed in this specification may further be used in a method for treating a viral infection in a patient by administering to the patient a therapeutically effective amount of a compound according to one or more embodiments disclosed in this specification. Examples of viruses that may cause viral infections to be treated include, but are not limited to: a polyomavirus, such as John Cunningham Virus (JCV), Simian virus 40 (SV40), or BK Virus (BKV); influenza, Human Immunodeficiency Virus type 1 (HIV-1), Human Papilloma Virus (HPV), adenovirus, Epstein-Barr Virus (EBV), Hepatitis C Virus (HCV), Molluscum contagiosum virus (MCV); Human T-lymphotropic virus type 1 HTLV-1), Herpes Simplex Virus type 1 (HSV-1), cytomegalovirus (CMV), hepatitis B virus, Bovine papillomavirus (BPV-1), human T-cell lymphotropic virus type 1, Japanese encephalitis virus, respiratory syncytial virus (RSV), and West Nile virus.

The compounds or pharmaceutical compositions according to one or more embodiments disclosed in this specification may further be used in a method for treating a betacoronavirus infection in a patient by administering to the patient a therapeutically effective amount of a compound or pharmaceutical composition according to one or more embodiments disclosed in this specification.

The compounds according to one or more embodiments disclosed in this specification may further be used in the preventing of a betacoronavirus infection in a patient by administering to the patient a prophylactically effective amount of a compound or pharmaceutical composition according to one or more embodiments disclosed in this specification.

The compounds according to one or more embodiments disclosed in this specification may be used for the manufacture of a medicament for the treatment or prophylaxis of a betacoronavirus infection.

In certain embodiments betacoronavirus is selected from the group consisting of Severe Acute Respiratory Syndrome coronavirus SARS-CoV, Middle East Respiratory Syndrome MERS-CoV, and severe acute respiratory syndrome coronavirus 2 (SARS-CoV-2; originally known as nCoV-2019).

In certain embodiments the betacoronavirus is SARS-CoV.

In certain embodiments the betacoronavirus is SARS-CoV-2.

Serine/Threonine phosphatases, including PP2A may be involved in modulation of synaptic plasticity. Decreased PP2A activity is associated with maintenance of Long Term Potentiation (LTP) of synapses, thus treatment PP2A modulators such as those described here may reverse synaptic LTP. Psychostimulant drugs of abuse such as cocaine and methamphetamine are associated with deleterious synaptic LTP, which may underlie the pathology of addiction and relapse therefore PP2A modulators described here may be useful as treatments for psychostimulant abuse.

### Pharmaceutical Compositions, Formulations, and Routes of Administration

The present disclosure relates also to a pharmaceutical composition comprising a compound according to one or more embodiments described in this specification, for example a compound of Formula I, an enantiomer, a diastereomer, a tautomer or a pharmaceutically acceptable salt thereof, and pharmaceutically acceptable carrier.

In certain embodiments, the pharmaceutical composition comprises a compound of Formula (I) or a pharmaceutically acceptable salt thereof, together with one or more pharmaceutically carriers thereof and optionally one or more other therapeutic ingredients. The carrier(s) may be “acceptable” in the sense of being compatible with the other ingredients of the formulation and not deleterious to the recipient thereof.

The pharmaceutical compositions may be manufactured in any manner known in the art, e.g., by means of conventional mixing, dissolving, granulating, dragee-making, levigating, emulsifying, encapsulating, entrapping or compression processes. “pharmaceutically acceptable carrier” may refer to an excipient, carrier or adjuvant that can be administered to a patient, together with at least one therapeutic compound, and which does not destroy the pharmacological activity thereof and is generally safe, nontoxic and neither biologically nor otherwise undesirable when administered in doses sufficient to deliver a therapeutic amount of the compound.

The pharmaceutical formulations may include those suitable for oral, parenteral (including subcutaneous, intradermal, intramuscular, intravenous, intraarticular, and intramedullary), intraperitoneal, transmucosal, transdermal, intranasal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of Formula (I) or a pharmaceutically acceptable salt, ester, amide, solvate, or enantiomer or diastereomer or tautomer thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds of Formula (I) suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds of Formula (I) may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, a compound of Formula (I) may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

In certain embodiments, compounds as disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 10% w/w of the formulation.

For administration by inhalation, compounds of Formula (I) may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds disclosed herein may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Intranasal delivery, in particular, may be useful for delivering compounds to the CNS. It had been shown that intranasal drug administration is a noninvasive method of bypassing the blood-brain barrier (BBB) to deliver neurotrophins and other therapeutic agents to the brain and spinal cord. Delivery from the nose to the CNS occurs within minutes along both the olfactory and trigeminal neural pathways. Intranasal delivery occurs by an extracellular route and does not require that drugs bind to any receptor or undergo axonal transport. Intranasal delivery also targets the nasal associated lymphatic tissues (NALT) and deep cervical lymph nodes. In addition, intranasally administered therapeutics are observed at high levels in the blood vessel walls and perivascular spaces of the cerebrovasculature. Using this intranasal method in animal models, researchers have successfully reduced stroke damage, reversed Alzheimer's neurodegeneration, reduced anxiety, improved memory, stimulated cerebral neurogenesis, and treated brain tumors.

In certain embodiments, unit dosage formulations are those containing an effective dose or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

### Combination Therapies

In certain instances, it may be appropriate to administer at least one of the compounds of Formula (I) (an enantiomer, a diastereomer, a tautomer or a pharmaceutically acceptable salt thereof) in combination with another therapeutic agent. By way of example only, if one of the side effects experienced by a patient upon receiving one of the compounds herein for the treatment of cancer is nausea, then it may be appropriate to administer an antiemetic agent in combination. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for cancer involving administration of one of the compounds described herein, increased therapeutic benefit may result by also providing the patient with another therapeutic agent for cancer. In any case, regardless of the disease, disorder or condition being treated, the overall benefit experienced by the patient may simply be additive of the two therapeutic agents or the patient may experience a synergistic benefit.

The instant compounds may be particularly useful in combination with therapeutic and/or anti-cancer agents. Thus, the present disclosure provides a combination of compounds of Formula (I) are used in a combination with therapeutic and/or anti-cancer agents for simultaneous, separate or sequential administration. The compounds of Formula (I) and the other anticancer agent can act additively or synergistically. A synergistic combination of the present compounds and another anticancer agent might allow the use of lower dosages of one or both of these agents and/or less frequent dosages of one or both of the instant compounds and other anticancer agents and/or to administer the agents less frequently can reduce any toxicity associated with the administration of the agents to a patient without reducing the efficacy of the agents in the treatment of cancer. In addition, a synergistic effect might result in the improved efficacy of these agents in the treatment of cancer and/or the reduction of any adverse or unwanted side effects associated with the use of either agent alone.

The therapeutic agent and/or anti-cancer agent can be administered according to therapeutic protocols well known in the art. It will be apparent to those skilled in the art that the administration of the therapeutic agent and/or anti-cancer agent can be varied depending on the disease being treated and the known effects of the anti-cancer agent on that disease. Also, in accordance with the knowledge of the skilled clinician, the therapeutic protocols (e.g., dosage amounts and times of administration) can be varied in view of the observed effects of the administered therapeutic agents (i.e., anti-neoplastic agent or radiation) on the patient, and in view of the observed responses of the disease to the administered therapeutic agents, and observed adverse effects.

In certain embodiments, the compounds according to one or more embodiments disclosed in this specification, for example compounds of Formula I, may be administered in combination with one or more agent selected from aromatase inhibitors, anti-estrogens, anti-progesterons, anti-androgens, or gonadorelin agonists, anti-inflammatory agents, antihistamines, anti-cancer agent, inhibitors of angiogenesis, topoisomerase 1 and 2 inhibitors, microtubule active agents, alkylating agents, antineoplastic, antimetabolite, dacarbazine (DTIC), platinum containing compound, lipid or protein kinase targeting agents, protein or lipid phosphatase targeting agents, anti-angiogenic agents, agents that induce cell differentiation, bradykinin 1 receptor and angiotensin II antagonists, cyclooxygenase inhibitors, heparanase inhibitors, lymphokines or cytokine inhibitors, bisphosphanates, rapamycin derivatives, anti-apoptotic pathway inhibitors, apoptotic pathway agonists, PPAR agonists, HSP90 inhibitor, smoothened antagonist, inhibitors of Ras isoforms, telomerase inhibitors, protease inhibitors, metalloproteinase inhibitors, aminopeptidase inhibitors, imununomodulators, therapeutic antibody and a protein kinase inhibitor, e.g., a tyrosine kinase or serine/threonine kinase inhibitor.

In certain embodiments, the combination of a compound of Formula I and an anti-cancer agent is provided for simultaneous, separate or sequential administration.

A person of ordinary skill in the art would be able to discern which combinations of agents would be useful based on the particular characteristics of the drugs and the cancer involved. Classes of such agents include the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, HIV protease inhibitors, reverse transcriptase inhibitors, inhibitors of cell proliferation and survival signaling, bisphosphonates, aromatase inhibitors, siRNA therapeutics, γ-secretase inhibitors, agents that interfere with receptor tyrosine kinases (RTKs), agents that interfere with cell cycle checkpoints, PARP inhibitors, HDAC inhibitors, Smo antagonists (HH inhibitors), HSP90 inhibitors, CYP17 inhibitors, 3rd generation AR antagonists, JAK inhibitors e.g. Ruxolitinib (trade name Jakafi), and BTK kinase inhibitors.

Anticancer agents suitable for use in the combination therapy with compounds as disclosed herein include, but are not limited to:
1) alkaloids and natural product drugs, including, microtubule inhibitors (e.g., Vincristine, Vinblastine, and Vindesine, and vinorelbine etc.), microtubule stabilizers (e.g., Paclitaxel [Taxol], and Docetaxel, Taxotere, etc.), and chromatin function inhibitors, including, topoisomerase inhibitors, such as, epipodophyllotoxins (e.g., Etoposide [VP-161, and Teniposide [VM-261, etc.), and agents that target topoisomerase I (e.g., Camptothecin, topotecan (Hycamtin) and Irinotecan [CPT-11], rubitecan (Orathecin) etc.);
2) covalent DNA-binding agents [alkylating agents], including, nitrogen mustards (e.g., Mechloretharnine, chlormethine, Chlorambucil, Cyclophosphamide, estramustine (Emcyt, Estracit), ifosfamide, Ifosphamide, melphalan (Alkeran) etc.); alkyl sulfonates like Busulfan [Myleran], nitrosoureas (e.g., Carmustine or BCNU (bis-chloroethylnitrosourea), fotemustine Lomustine, and Semustine, streptozocin etc.), and other alkylating agents (e.g., Dacarbazine, procarbazine ethylenimine/methylmelamine, thriethylenemelamine (TEM), triethylene thiophosphoramide (thiotepa), hexamethylmelamine (HMM, altretamine), and Mitocycin, uramustine etc.) including Temozolomide (brand names Temodar and Temodal and Temcad), altretamine (also hexalen) and mitomycin; and
3) noncovalent DNA-binding agents [antitumor antibiotics], including nucleic acid inhibitors (e.g., Dactinomycin [Actinomycin Dl, etc.), anthracyclines (e.g., Daunorubicin [Daunomycin, and Cerubidine], Doxorubicin [Adrianycin], epirubicin (Ellence), and Idarubicin [Idamycin], valrubicin (Valstar) etc.), anthracenediones (e.g., anthracycline analogues, such as, [Mitoxantrone], etc.), bleomycins (Blenoxane), etc., amsacrine and plicamycin (Mithramycin), dactinomycin, mitomycin C.

In certain embodiments, a patient with cancer is treated with a combination of a compound Formula (I) and radiation therapy. In certain embodiments, the method comprises administering to a patient with cancer a therapeutically effective amount of a compound of the disclosure, and adjunctively treating the patient with an effective amount of radiation therapy. In certain embodiments, the compound is administered to the patient in need thereof prior to, concurrently with, or subsequent to the treatment with radiation.

In certain embodiments, the compounds or the pharmaceutical composition may further comprise or be administered in combination with one or more other antiviral agents including, but not limited to, oseltamivir phosphate, zanamivir or Virazole®, Remdesivir, Vidarabine, Acyclovir, Ganciclovir, Valganciclovir, Valacyclovir, Cidofovir, Famciclovir, Ribavirin, Amantadine, Rimantadine, Interferon, Oseltamivir, Palivizumab, Rimantadine, Zanamivir, nucleoside-analog reverse transcriptase inhibitors (NRTI) such as Zidovudine, Didanosine, Zalcitabine, Stavudine, Lamivudine and Abacavir, non-nucleoside reverse transcriptase inhibitors (NNRTI) such as Nevirapine, Delavirdine and Efavirenz, protease inhibitors such as Saquinavir, Ritonavir, Indinavir, Nelfinavir, Amprenavir, and other known antiviral compounds and preparations.

In certain embodiments, the compounds or the pharmaceutical compositions may be co-administered with one or more antiviral agents. The compounds or the pharmaceutical compositions of the present invention may be administered in any order.

### Controlled Release Formulations and Drug Delivery Systems

In certain embodiments, the compositions and/or formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In certain embodiments of the invention, the compounds useful within the invention are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

### Administration/Dosing

The compound may be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

In certain embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any patient will be determined by the attending physician taking all other factors about the patient into account.

Compounds according to one or more embodiments disclosed in this specification may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compound which is effective at such dosage or as a multiple of the same, for instance, units containing 5 mg to 500 mg, usually around 10 mg to 200 mg.

Compounds of the invention for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the invention is from about 0.5 μg and about 5,000 mg. In some embodiments, a dose of a compound of the invention used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

The amount of active ingredient that may be combined with the carrier materials to produce a single dosage form will vary depending upon the host treated and the particular mode of administration.

The compound of Formula (I) can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

### LCMS Conditions

Method A: HPLC Alliance 2695 system employing Gemini NX-C18 Phenomenex (30×2 mm), 3 μm column, with eluents comprising eluent A=H₂O+0.05% TFA (v/v); and eluent B=CH₃CN+0.035% TFA. Oven temperature: 55° C.; Gradient: t_{0 min}=2% B; t_{2.5 min}=98% B; t_{3.5 min}=98% B; t_{3.6 min}=2% B; t_{5 min}=5% B (v/v). Flow rate: 0.9 mL/min. Positive electrospray ES+; Capillary: 3.5 kV; Cone voltage: 15 V.

Method B: Waters Acquity UPLC system employing Waters Acquity UPLC CSH C18 (50×2.1 mm), 1.7 μm column, with eluents comprising: eluent A=H₂O+0.02% HCOOH (v/v); and eluent B=CH₃CN+0.02% HCOOH (v/v). Oven temperature: 55° C.; Gradient: t_{0 min}=0.2% B; t_{4 min}=98% B; t_{4.5 min}=98% B; t_{4.6 min}=2% B; t_{5 min}=2% B. Flow rate: 1 mL/min. Negative electrospray ES−; Capillary: 3 kV; Cone voltage: 15 V.

Method C: Waters Acquity UPLC system employing Waters Acquity UPLC CSH C18 (2.1×50 mm), 1.7 μm column, with eluents comprising: eluent A=H₂O+0.02% HCOOH; and eluent B=CH₃CN+0.02% HCOOH. Oven Temperature: 55° C.; Gradient: t₀=2% B; t_{4 min}=98% B; t_{4.5 min}=98% B; t_{4.6 min}=2% B; and t_{5.0 min}=2% B. Flow rate: 1 mL/min. Electrospray Ionization Mode; Capillary: 3 kV; Cone voltage: 15/30 V.

Method D: Waters Acquity UPLC system employing Waters Acquity UPLC CSH C18 (2.1×50 mm), 1.7 μm column, with eluents comprising: eluent A (H₂O+0.05% TFA) and eluent B (CH₃CN+0.035% TFA). Oven temperature: 55° C. Gradient: t₀=2% B; t_{4 min}=98% B; t_{4.5 min}=98% B; t_{4.6 min}=2% B; and t_{5 min}=2% B. Flow rate: 1 mL/min. Electrospray Ionization Mode; Capillary: 3 kV; Cone voltage: 15/30 V.

Method E: Waters Acquity UPLC system employing Waters Acquity UPLC CSH C18 (2.1×50 mm), 1.7 μm column, A=H₂O+0.05% TFA (v/v); and eluent B=CH₃CN+0.035% TFA. Oven temperature: 55° C.; Gradient: t_{0 min}=2% B; t_{1 min}=98% B; t_{1.5 min}=98% B; t_{1.52 min}=2% B; t_{1.7 min}=2% B (v/v). Flow rate: 0.8 mL/min. Positive electrospray ES+; Capillary: 0.8 kV; Cone voltage: 15 V.

Method F: Waters Acquity UPLC system employing Waters Acquity UPLC CSH C18 (2.1×50 mm), 1.7 μm column, A=H₂O+0.05% TFA (v/v); and eluent B=CH₃CN+0.035% TFA. Oven temperature: 55° C.; Gradient: t_{0 min}=2% B; t_{1 min}=98% B; t_{1.5 min}=98% B; t_{1.52 min}=2% B; t_{1.7 min}=2% B (v/v). Flow rate: 0.8 mL/min. Positive electrospray ES+; Capillary: 0.8 kV; Cone voltage: 10 V.

Method G: Waters Acquity UPLC system employing Waters Acquity UPLC CSH C18 (2.1×100 mm), 1.7 μm column, with eluents comprising: eluent A (H₂O+0.02% HCOOH) and eluent B (CH₃CN+0.02% HCOOH). Oven temperature: 55° C. Gradient: t₀=2% B; t_{15 min}=98% B; t_{15.2 min}=2% B; and t_{18 min}=2% B. Flow rate: 0.7 mL/min. Electrospray Ionization Mode; Capillary: 3 kV; Cone voltage: 15/30 V.

Method H: Waters Acquity UPLC system employing Waters Acquity UPLC CSH C18 (50×2.1 mm), 1.7 μm column, with eluents comprising: eluent A=H₂O+0.02% HCOOH (v/v); and eluent B=CH₃CN+0.02% HCOOH (v/v). Oven temperature: 55° C.; Gradient: t_{0 min}=0.2% B; t_{4 min}=98% B; t_{4.5 min}=98% B; t_{4.6 min}=2% B; t_{5 min}=2% B. Flow rate: 1 mL/min. Negative electrospray ES−; Capillary: 3 kV; Sample Cone: 15/30 V.

Method I: Waters Acquity UPLC system employing Waters Acquity UPLC CSH C18 (50×2.1 mm) 1.7 μm column, with eluents comprising: eluent A (H₂O+0.05% TFA) and eluent B (CH₃CN+0.035% TFA). Oven temperature: 55° C. Gradient: t₀=2% B; t_{2.4 min}=98% B, t_{3.0 min}=98% B, t_{3.03 min}=2% B; t_{3.5 min}=2% B. Flow rate: 0.8 mL/min. Ionization Mode: electrospray; Capillary: 0.8 kV; Sample voltage: 10 V; Mass range: 120-1250; Gas pressure=40 psi; Gain=500; Nebulizer mode=cooling; Drift tube temperature=50 55° C.

### EXAMPLES

Various embodiments of the present application can be better understood by reference to the following Examples which are offered by way of illustration. The scope of the present application is not limited to the Examples given herein.

As described herein, “Diastereomer I” or “Enantiomer I” refer to the first diastereomer or enantiomer, respectively, eluted from a chiral or achiral column under the specific analytical conditions detailed for examples provided elsewhere herein.

As described herein, “Diastereomer II” or “Enantiomer II” refer to the second diastereomer or enantiomer, respectively, eluted from the chiral or achiral column under the specific analytical conditions detailed for examples provided elsewhere herein. Such nomenclature does not imply or impart any particular relative and/or absolute configuration for these compounds.

### Example 1: Synthesis of Starting Materials and Common Intermediates

### Synthesis of 1-(3,4-dichlorophenyl)-N,N′-dimethyl-ethane-1,2-diamine

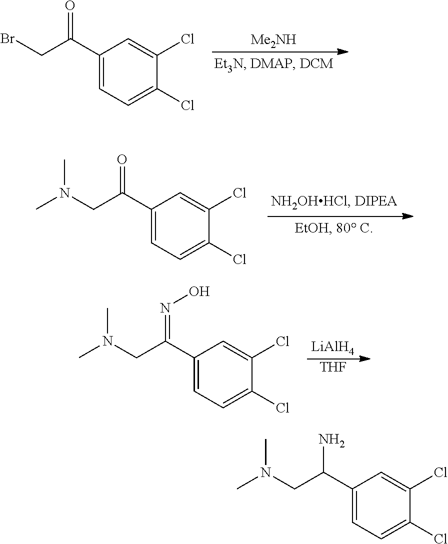

### Step 1: Synthesis of 1-(3,4-dichlorophenyl)-2-(dimethylamino)ethan-1-one

In a round-bottomed flask, to a stirred solution of 2-bromo-1-(3,4-dichlorophenyl)ethanone (97%, 15.00 g, 54.3 mmol) in anhydrous DCM (108.61 mL) at 0° C. under nitrogen were added successively 2 M dimethylamine in THF (49 mL, 97.7 mmol), triethylamine (8.3 mL, 59.7 mmol) and DMAP (332 mg, 2.72 mmol). The reaction mixture was warmed and stirred at room temperature overnight. The reaction mixture was quenched with water (400 mL) and ethyl acetate (400 mL) was added. The aqueous layer was extracted with ethyl acetate (2×250 mL). The combined organic layers were washed with a saturated aqueous solution of sodium chloride, dried over sodium sulfate, filtered and concentrated under reduced pressure. The crude was purified by flash chromatography on silica gel using a gradient of MeOH in DCM from 0% to 2% to afford the title compound as a brown oil (6.77 g, 54% yield, tᵣ=0.51 min). LCMS (Method F): m/z found 232.1 [M+H]⁺; ¹H-NMR (DMSO-d₆, 400 MHz) δ (ppm) 8.18 (d, J=2.0 Hz, 1H), 7.94 (dd, J=8.4, 2.0 Hz, 1H), 7.80 (d, J=8.4 Hz, 1H), 3.75 (s, 2H), 2.24 (s, 6H).

### Step 2: Synthesis of 1-(3,4-dichlorophenyl)-2-(dimethylamino)ethanone oxime

A sealed vial was charged with 1-(3,4-dichlorophenyl)-2-(dimethylamino)ethan-1-one (99%, 6.77 g, 28.9 mmol), hydroxylamine hydrochloride (4.01 g, 57.7 mmol) and DIPEA (11 mL, 60.6 mmol) in anhydrous ethanol (127.46 mL). The reaction mixture was stirred at 80° C. for 4 h. The reaction mixture was concentrated under reduced pressure and dried under vacuum to afford the title compound as a brown oil which was used without purification (16.43 g, quantitative yield, 44% purity, tᵣ=0.53 min). LCMS (Method F): m/z found 247.1 [M+H]⁺; ¹H-NMR (DMSO-d₆, 400 MHz) δ (ppm) 7.90 (dd, J=20.8, 1.9 Hz, 1H), 7.75-7.57 (m, 2H), 4.42-4.28 (m, 1H), 3.51-3.35 (m, 2H), 2.19 (d, J=3.0 Hz, 5H).

### Step 3: Synthesis of 1-(3,4-dichlorophenyl)-N′,N′-dimethyl-ethane-1,2-diamine

In three-neck round-bottom flask, to a stirred solution of 1-(3,4-dichlorophenyl)-2-(dimethylamino)ethanone oxime (44%, 16.43 g, 29.3 mmol) in anhydrous THF (128.52 mL) at 0° C. under nitrogen was added dropwise 2.4 M Lithium aluminum hydride in THF (30 mL, 73.1 mmol) over 40 min (temperature went up to 8° C.). The reaction mixture was stirred at 75° C. (65° C. in the mixture) for 3 h. The reaction mixture was cooled to 0° C., quenched with water (2.77 mL, same weight as LAH) via dropwise addition. NaOH (2.8 mL, 2.78 mmol, 1 M) and water (three times the weight of LAH) were added. The resulting heterogeneous mixture was stirred at room temperature overnight, filtered, and rinsed with THF. The filtrate was dried over Na₂SO₄, filtered, and concentrated under reduced pressure. The crude was purified by flash chromatography on silica gel using a gradient of methanol (0.7N NH₃) in dichloromethane from 1% to 4%. The desired fractions were combined and concentrated in vacuo to afford the title compound as an orange oil (3.484 g, 48% yield, tᵣ=0.39 min). LCMS (Method E): m/z found 233.1 [M+H]⁺; ¹H-NMR (DMSO-d₆, 400 MHz) δ (ppm) 7.64 (d, J=2.5 Hz, 1H), 7.55 (dd, J=8.3, 6.2 Hz, 1H), 7.35 (dd, J=8.3, 4.2, 2.0 Hz, 1H), 3.98 (dd, J=8.8, 5.4 Hz, 1H), 2.31-1.86 (m, 11H).

### Synthesis of 1-(4-chlorophenyl)-2-(4-methylpiperazin-1-yl)ethanamine

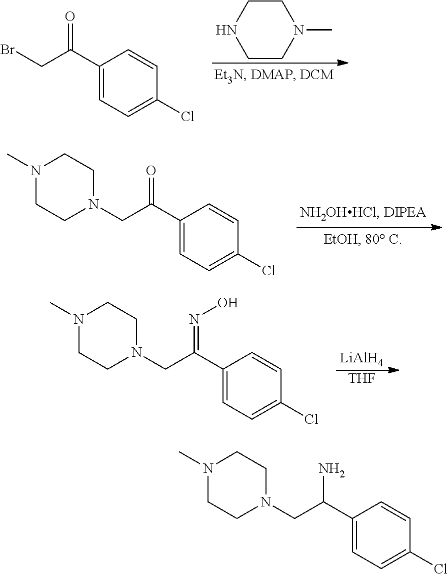

### Step 1: Synthesis of 1-(4-chlorophenyl)-2-(4-methylpiperazin-1-yl)ethenone

In a sealed tube under nitrogen, a solution of 2-bromo-1-(4-chlorophenyl)ethanone (777 mg, 3.33 mmol) and DMAP (20 mg, 0.166 mmol) in dry DCM (7.77 mL) was stirred at rt. 1-methylpiperazine (664 μL, 5.99 mmol) then triethylamine (510 μL, 3.66 mmol) were added and the solution was stirred at rt for 2 days. The mixture was washed with a half saturated aqueous solution of NaHCO₃. The aqueous layer was extracted twice with DCM. The combined organic layers were washed with brine, dried over sodium sulfate, filtered, and concentrated. The crude was purified by flash chromatography on silica gel using a gradient of MeOH in DCM from 2% to 10% to afford the title compound as a brown solid. (612.4 mg, 73% yield, tᵣ=0.47 min). LCMS (Method F): m/z found 253.3 [M+H]⁺; ¹H-NMR (DMSO-d₆, 400 MHz) δ (ppm) 8.04-7.98 (m, 2H), 7.65-7.56 (m, 2H), 3.82 (s, 2H), 2.64-2.51 (m, 4H), 2.49-2.31 (m, 4H), 2.23 (s, 3H).

### Step 2: Synthesis of 1-(4-chlorophenyl)-2-(4-methylpiperazin-1-yl)ethanone oxime

A sealed vial was charged with 1-(4-chlorophenyl)-2-(4-methylpiperazin-1-yl)ethanone (612 mg, 2.42 mmol), hydroxylamine hydrochloride (337 mg, 4.84 mmol) and DIPEA (888 μL, 5.09 mmol) in anhydrous EtOH (10.578 mL). The reaction mixture was stirred at 80° C. for 18 h. The reaction mixture was concentrated under reduced pressure and dried under vacuum to afford the title compound as a yellow solid (1.5 g, quantitative yield, tᵣ=0.48 min). LCMS (Method F): m/z found 268.3 [M+H]⁺; ¹H-NMR (DMSO-d₆, 400 MHz) δ (ppm) 7.70 (dd, J=46.1, 8.7 Hz, 2H), 7.45 (dd, J=9.0 Hz, 2H), 3.68-3.53 (m, 6H), 3.01-2.82 (m, 4H), 2.71 (s, 3H), 2.49-2.26 (m, 2H).

### Step 3: Synthesis of 1-(4-chlorophenyl)-2-(4-methylpiperazin-1-yl)ethanamine

In a vial under atmospheric pressure, to a stirred solution of 1-(4-chlorophenyl)-2-(4-methylpiperazin-1-yl)ethanone oxime (648 mg, 2.42 mmol) in anhydrous THF (16.143 mL) at 0° C. under nitrogen was added 2.4 M LAH in THF (2.5 mL, 6.05 mmol). The reaction mixture was stirred at 70° C. for 3 h. The reaction mixture was quenched at 0° C. with water (0.31 mL, same weight as LAH used). 1 M sodium hydroxide (0.33 mL, 0.329 mmol) and water (three times the weight of LAH used) were added. The resulting heterogenous mixture was stirred at room temperature for 15 min, dried over sodium sulfate, filtered, rinsed with THF, and concentrated under reduced pressure. The crude product was solubilized in anhydrous THF (16.143 mL), put under nitrogen at 0° C., and 2.4 M LAH in THF (2.0 mL, 4.84 mmol) was added. The reaction mixture was stirred at 70° C. for 3 h. The reaction mixture was quenched at 0° C. with water (0.18 mL, same weight as LAH used). 1 M sodium hydroxide (0.18 mL, 0.329 mmol) was added and water (three times the weight of LAH used). The resulting heterogenous mixture was stirred at room temperature for 15 min, dried over sodium sulfate, filtered, rinsed with THF, and concentrated under reduced pressure. The crude was purified by flash chromatography on silica gel using a gradient of (0.7N NH₃) methanol in dichloromethane from 2% to 8% to afford the title compound as a pale yellow solid (174 mg, 28% yield, tᵣ=0.76 min). LCMS (Method F): m/z found 254.3 [M+H]⁺; 1H-NMR (DMSO-d₆, 400 MHz) δ (ppm) 7.44-7.37 (m, 2H), 7.37-7.29 (m, 2H), 4.03 (dd, J=9.4, 4.7 Hz, 1H), 2.50 (m, 2H), 2.43-2.18 (m, 8H), 2.15 (s, 3H), 1.88 (s, 2H).

### Synthesis of 1-(3,4-dichlorophenyl)-2-(4-methylpiperazin-1-yl)ethanamine

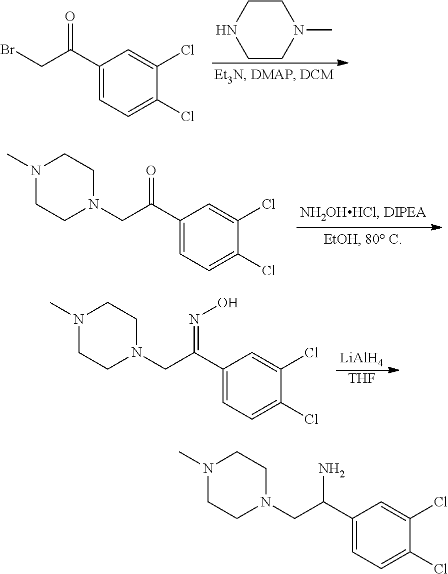

### Step 1: Synthesis of 1-(3,4-dichlorophenyl)-2-(4-methylpiperazin-1-yl)ethanone

In a sealed tube under nitrogen, a solution of 2-bromo-1-(3,4-dichlorophenyl)ethanone (1.00 g, 3.73 mmol) and DMAP (23 mg, 0.187 mmol) in dry DCM (10 mL) was stirred at rt. 1-methylpiperazine (745 μL, 6.72 mmol) then triethylamine (572 μL, 4.11 mmol) were added and the solution was stirred at rt for 16 h. The mixture was washed with a half saturated aqueous solution of NaHCO₃. The aqueous layer was extracted twice with DCM. The combined organic layers were washed with brine, dried over sodium sulfate, filtered, and concentrated. The crude was purified by flash chromatography on silica gel using a gradient of MeOH in DCM from 2% to 10% to afford the title compound as an off-white solid. (607 mg, 56% yield, tᵣ=0.53 min). LCMS (Method F): m/z found 287.2 [M+H]⁺; ¹H-NMR (DMSO-d₆, 400 MHz) δ (ppm) 8.19 (d, J=2.0 Hz, 1H), 7.95 (dd, J=8.4, 2.0 Hz, 1H), 7.81 (d, J=8.4 Hz, 1H), 3.83 (s, 2H), 2.56-2.51 (m, 4H), 2.45-2.25 (m, 4H), 2.20 (s, 3H).

### Step 2: Synthesis of 1-(3,4-dichlorophenyl)-2-(4-methylpiperazin-1-yl)ethanone oxime

A sealed vial was charged with 1-(3,4-dichlorophenyl)-2-(4-methylpiperazin-1-yl)ethanone (607 mg, 2.11 mmol), hydroxylamine hydrochloride (294 mg, 4.23 mmol) and DIPEA (775 μL, 4.44 mmol) in anhydrous EtOH (9.2336 mL). The reaction mixture was stirred at 80° C. overnight. The reaction mixture was concentrated under reduced pressure and dried under vacuum to afford the title compound as a yellow solid (1.5 g, quantitative yield, tᵣ=0.53 and 0.54 min). LCMS (Method F): m/z found 302.3 [M+H]⁺; ¹H-NMR (DMSO-d₆, 400 MHz) δ (ppm) 7.91 (dd, J=26.3, 1.9 Hz, 1H), 7.76-7.54 (m, 2H), 3.61-3.49 (m, 4H), 3.06 (d, J=6.4 Hz, 4H), 2.45 (bs, 5H).

### Step 3: Synthesis of 1-(3,4-dichlorophenyl)-2-(4-methylpiperazin-1-yl)ethanamine

In a vial under atmospheric pressure, to a stirred solution of 1-(3,4-dichlorophenyl)-2-(4-methylpiperazin-1-yl)ethanone oxime (639 mg, 2.11 mmol) in anhydrous THF (14.091 mL) at 0° C. under nitrogen was added 2.4 M LAH in THF (2.2 mL, 5.28 mmol). The reaction mixture was stirred at 70° C. for 3 h. The reaction mixture was quenched at 0° C. with water (0.31 mL, same weight as LAH used). 1 M sodium hydroxide (0.33 mL, 0.329 mmol) and water (three times the weight of LAH used) were added. The resulting heterogenous mixture was stirred at room temperature for 15 min, dried over sodium sulfate, filtered, rinsed with THF, and concentrated under reduced pressure. The crude product was purified by flash chromatography on silica gel using a gradient of (0.7N NH₃) methanol in dichloromethane from 2% to 8% to afford the title compound as a yellow oil (282 mg, 42% yield, tᵣ=0.92 min). LCMS (Method F): m/z found 288.3 [M+H]⁺; ¹H-NMR (DMSO-d₆, 400 MHz): δ (ppm) 7.65 (d, J=2.0 Hz, 1H), 7.55 (d, J=8.3 Hz, 1H), 7.36 (dd, J=8.3, 2.0 Hz, 1H), 4.04 (dd, J=8.9, 5.3 Hz, 1H), 2.50-2.43 (m, 2H), 2.28 (6, J=17.4, 12.1, 7.1 Hz, 8H), 2.20-2.07 (m, 5H).

### Synthesis of [1-(3,4-dichlorophenyl)cyclopropyl]methanamine

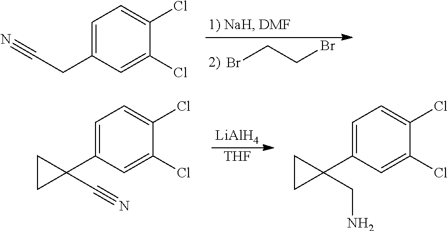

### Step 1: Synthesis of 1-(3,4-dichlorophenyl)cyclopropane-1-carbonitrile

In a three-neck round-bottomed flask, to a stirred suspension of NaH (60%, 116 mg, 2.90 mmol) in anhydrous DMF (2.8 mL) at 0° C. under nitrogen was added dropwise a solution of (3,4-dichlorophenyl)acetonitrile (98%, 200 mg, 1.05 mmol) in anhydrous DMF (1 mL). The reaction mixture was stirred at 0° C. for 15 min (until no gas production was observed). A solution of 1,2-dibromoethane (136 μL, 1.58 mmol) in anhydrous DMF (1 mL) was added dropwise at 0° C., then the reaction mixture was allowed to warm up to room temperature and stirred for 3 h. The reaction mixture was quenched with water (25 mL) and EtOAc was added. The layers were separated and the aqueous layer was washed twice with EtOAc. The combined organic layers were washed with a saturated aqueous solution of NaCl, dried over sodium sulfate, filtered and concentrated under reduced pressure. The crude was purified by flash chromatography on silica gel using a gradient of EtOAc in cyclohexane from 0% to 50%. The desired fractions were combined and concentrated to afford the title compound as a pale yellow solid (182.7 mg, 82% yield, tᵣ=0.92 min). LCMS (Method F); ¹H-NMR (DMSO-d₆, 400 MHz) δ (ppm) 7.66 (d, J=8.4 Hz, 1H), 7.55 (d, J=2.3 Hz, 1H), 7.38 (dd, J=8.4, 2.3 Hz, 1H), 1.83-1.77 (m, 2H), 1.63-1.57 (m, 2H).

### Step 2: Synthesis of [1-(3,4-dichlorophenyl)cyclopropyl]methanamine

In round-bottom flask, to a stirred solution of 1-(3,4-dichlorophenyl)cyclopropanecarbonitrile (183 mg, 0.861 mmol) in anhydrous THF (3.6904 mL) at 0° C. under nitrogen was added dropwise 2.4 M LAH (0.90 mL, 2.16 mmol) (exothermic reaction with gas evolution). The reaction mixture was stirred at rt for 2 h. The reaction mixture was quenched at 0° C. with water (82 μL, same weight as LAH used) via dropwise addition. Next, 1 M sodium hydroxide (86 μL, 0.086 mmol) and water (three times the weight of LAH used, 250 μL) were added. The resulting heterogenous mixture was stirred at room temperature for 30 min, then Na₂SO₄ was added and the resulting suspension was filtered and rinsed with THF. The filtrate was concentrated under reduced pressure. The crude was purified by flash chromatography on silica gel using a gradient of MeOH (0.7N NH₃) in DCM from 0.5% to 10%. The desired fractions were combined and concentrated to afford the title compound as a colorless oil (141 mg, 76% yield, tᵣ=0.56 min). LCMS (Method F): m/z found 216.2 [M+H]⁺; ¹H-NMR (DMSO-d₆, 400 MHz) δ (ppm) 7.54-7.50 (m, 2H), 7.28 (dd, J=8.4, 2.2 Hz, 1H), 3.31 (s, 2H), 1.35 (s, 2H), 0.83-0.77 (m, 2H), 0.77-0.71 (m, 2H).

### Synthesis of 4-bromo-N-[1-(3,4-dichlorophenyl)-2-(dimethylamino)ethyl]benzenesulfonamide

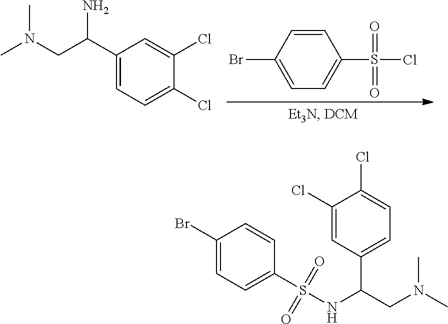

In a round-bottomed flask, to a stirred solution of 1-(3,4-dichlorophenyl)-N′,N′-dimethylethane-1,2-diamine (90%, 755 mg, 2.91 mmol) in anhydrous DCM (27 mL) at rt under nitrogen were added successively 4-bromobenzenesulfonyl chloride (98%, 760 mg, 2.91 mmol), and triethylamine (1.2 mL, 8.75 mmol). The solution was stirred at rt for 3.5 h and was diluted with water. The aqueous layer was extracted twice with DCM, the combined organic layers were washed with a saturated aqueous solution of NH₄Cl, a saturated aqueous solution of NaHCO₃, and a saturated aqueous solution of NaCl, then dried using a phase separator and concentrated. The crude was purified by flash chromatography on silica gel using a gradient of MeOH in DCM from 0.2% to 5%. The desired fractions were concentrated to afford the title compound as a yellow oil (747.7 mg, 56.733% yield, tᵣ=1.12 min). LCMS (Method F): m/z found 453.1 [M+H]⁺; ¹H-NMR (DMSO-d₆, 400 MHz) δ (ppm) 8.26 (s, 1H), 7.64-7.56 (m, 2H), 7.53-7.45 (m, 2H), 7.41 (d, J=8.3 Hz, 1H), 7.31 (d, J=2.0 Hz, 1H), 7.16 (dd, J=8.3, 2.1 Hz, 1H), 4.38-4.29 (m, 1H), 2.42 (dd, J=12.3, 8.3 Hz, 1H), 2.31 (dd, J=12.4, 6.9 Hz, 1H), 2.06 (s, 6H).

### Synthesis of 4-[[1-(3,4-dichlorophenyl)-2-(dimethylamino)ethyl]sulfamoyl]benzoic acid

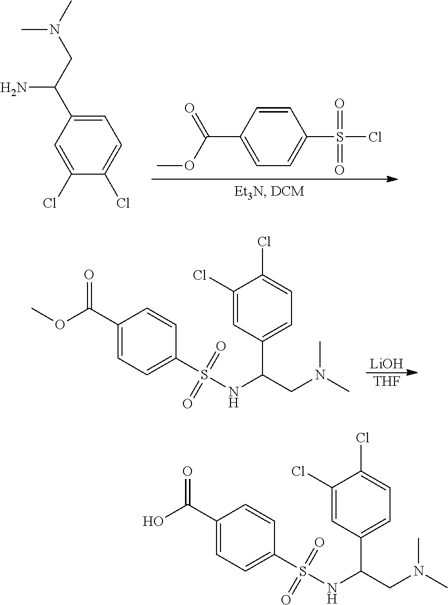

### Step 1: Synthesis of methyl 4-[[1-(3,4-dichlorophenyl)-2-(dimethylamino)ethyl]sulfamoyl]benzoate

In a sealed tube, methyl 4-(chlorosulfonyl)benzoate (93%, 720 mg, 2.85 mmol) was added to a stirred solution of 1-(3,4-dichlorophenyl)-N′,N′-dimethylethane-1,2-diamine (90%, 739 mg, 2.85 mmol) and triethylamine (1.2 mL, 8.56 mmol) in anhydrous DCM (25.004 mL). The solution was stirred at room temperature overnight. Water (6 mL) was added and the aqueous layer was extracted with dichloromethane (1×6 mL). The combined organic layers were washed with a saturated aqueous solution of sodium chloride, dried over sodium sulfate, filtered and concentrated under reduced pressure to afford a yellow solid (1.31 g). The crude product was purified by flash chromatography on silica gel (column 120 g, 50 μm) using a gradient of methanol in dichloromethane from 0% to 5%. The desired fractions were combined and concentrated to afford the title compound as a yellow solid (1.05 g, 69.119% yield, tᵣ=0.60 min). LCMS (Method F): m/z found 431.3 [M+H]⁺; ¹H-NMR (DMSO-d₆, 400 MHz) δ (ppm) 8.35 (s, 1H), 7.97-7.92 (m, 2H), 7.76-7.68 (m, 2H), 7.38 (d, J=8.3 Hz, 1H), 7.30 (d, J=2.0 Hz, 1H), 7.18-7.14 (m, 1H), 4.36 (t, J=7.3 Hz, 1H), 3.88 (s, 3H), 2.40 (ddd, J=12.8, 8.3, 4.5 Hz, 1H), 2.30 (dd, J=12.6, 6.9 Hz, 1H), 2.03 (s, 6H), 2.01 (s, 1H).

### Step 2: Synthesis of 4-[[1-(3,4-dichlorophenyl)-2-(dimethylamino)ethyl]sulfamoyl]benzoic acid

In a round-bottomed flask, to a stirred solution of methyl 4-[[1-(3,4-dichlorophenyl)-2-(dimethylamino)ethyl]sulfamoyl]benzoate (81%, 939 mg, 1.76 mmol) in THF (38.029 mL) at room temperature was added 0.5 M lithium hydroxide (8.7 mL, 4.37 mmol). The reaction mixture was stirred at room temperature overnight. The reaction mixture was concentrated under reduced pressure. The crude product was precipitated in DMSO, filtered and washed with water (three times) and diethyl ether (three times). The product was dried under reduced pressure to afford the title compound as a white powder (735 mg, 87.896% yield, tᵣ=0.51 min). LCMS (Method F): m/z found 417.3 [M+H]⁺; ¹H-NMR (DMSO-d₆, 400 MHz) δ (ppm) 7.91 (d, J=8.4 Hz, 2H), 7.69 (d, J=8.4 Hz, 2H), 7.39 (d, J=8.3 Hz, 1H), 7.31 (d, J=1.9 Hz, 1H), 7.16 (dd, J=8.3, 2.0 Hz, 1H), 4.36 (s, 1H), 2.41 (dd, J=12.4, 8.1 Hz, 1H), 2.31 (dd, J=12.5, 6.9 Hz, 1H), 2.04 (s, 6H).

### Synthesis of N-[1-(3,4-dichlorophenyl)-2-(dimethylamino)ethyl]-4-hydroxy-benzenesulfonamide

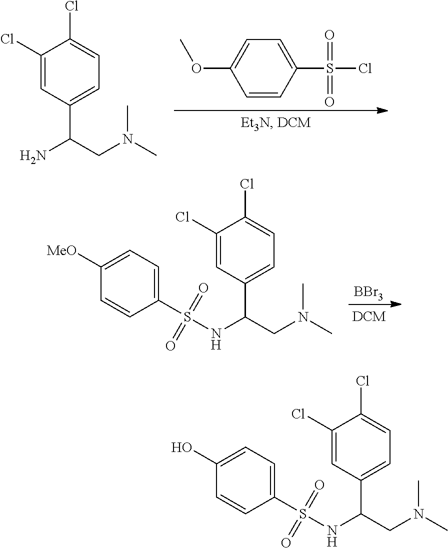

### Step 1: Synthesis of N-[1-(3,4-dichlorophenyl)-2-(dimethylamino)ethyl]-4-methoxy-benzenesulfonamide

In a round bottomed flask under a nitrogen atmosphere, 4-methoxybenzenesulfonyl chloride (99%, 1791 mg, 8.58 mmol) was added to a stirred solution of 1-(3,4-dichlorophenyl)-N′,N′-dimethylethane-1,2-diamine (2000 mg, 8.58 mmol) and triethylamine (4.8 mL, 34.3 mmol) in anhydrous DCM (57.19 mL). The solution was stirred at rt overnight then DCM and an half saturated solution of NaHCO₃ were added, and the aqueous layer was extracted twice with DCM. The combined organic layers were filtered through phase separator, then concentrated in vacuo, and purified by flash chromatography on silica gel using a gradient of MeOH in DCM from 0% to 4% to afford the title compound as a yellow oil (2.99 g, 86% yield, tᵣ=0.62 min). LCMS (Method F): m/z found 403.2 [M+H]⁺; ¹H-NMR (DMSO-d₆, 400 MHz) δ (ppm) 7.91 (d, J=7.7 Hz, 1H), 7.55-7.45 (m, 2H), 7.40 (d, J=8.3 Hz, 1H), 7.30 (d, J=2.0 Hz, 1H), 7.16 (dd, J=8.3, 2.1 Hz, 1H), 6.96-6.83 (m, 2H), 4.27 (q, J=7.4 Hz, 1H), 3.79 (s, 3H), 2.43-2.22 (m, 2H), 2.05 (s, 6H).

### Step 2: Synthesis of N-[1-(3,4-dichlorophenyl)-2-(dimethylamino)ethyl]-4-hydroxy-benzenesulfonamide

In a sealed vial, to a stirred solution of N-[1-(3,4-dichlorophenyl)-2-(dimethylamino)ethyl]-4-methoxy-benzenesulfonamide (99%, 2.99 g, 7.34 mmol) in DCM (29.357 mL) at 0° C. under nitrogen was added 1 M BBr₃ in dichloromethane (22 mL, 22.0 mmol). The solution was stirred at 0° C. for an hour, then 4 h at room temperature. Methanol (14.609 mL) was added and the mixture was stirred overnight at rt and concentrated in vacuo. Ethyl acetate and an half saturated solution of NaHCO₃ were added, layers were separated, and the aqueous layer was extracted once more with EtOAc. The combined organic layers were washed with brine, dried over Na₂SO₄ and concentrated in vacuo to afford the title compound as a beige powder (3.004 g, 98% yield, tᵣ=0.53 min). LCMS (Method F): m/z found 389.2 [M+H]⁺; ¹H-NMR (DMSO-d₆, 400 MHz) δ (ppm) 10.25 (s, 1H), 7.76 (s, 1H), 7.50-7.33 (m, 4H), 7.18 (dd, J=8.3, 2.0 Hz, 1H), 6.75 (dt, 2H), 4.30-4.16 (m, 1H), 2.32 (d, J=5.9 Hz, 2H), 2.01 (s, 6H).

### Example 2: Synthesis of Compounds 1-115

### Procedure for Sulfonamide Synthesis—Sulfonyl Chloride and Amine Couplings

Each vial was charged with an amine (0.4221 mmol, 1.1 eq) and Et₃N (1.5348 mmol, 4 eq, 214 μL). A solution of 4-(trifluoromethoxy)benzenesulfonyl chloride in DCM (0.384 mmol, 1.0 eq, 2 mL, 0.192 mmol/mL) was distributed in each vial. The reaction mixture was stirred at 25° C. overnight. The reaction mixture was filtered on a 3 mL ChemElut cartridge (pre-treated with HCl 1M (3 mL)) and eluted with 2 ml of DCM. The obtained fraction was concentrated under nitrogen flow at rt for 2 h, then dried under vacuum at 40° C. overnight. Purification was performed, when necessary, by SCX, PE-AX or flash chromatography. SCX: ISOLUTE—SCX (1 g, 6 mL) cartridges were used. The cartridges were eluted with 10 mL of DCM, then a solution of crude diluted in 1 mL was filtered by gravity. The cartridge was eluted twice with 4 mL of DCM and once with 2 mL of 2 N NH₃ in MeOH. The desired fractions were concentrated under nitrogen flow at rt for 2 h, then dried under vacuum at 40° C. overnight. PE-AX: ISOLUTE—PE-AX (1 g, 6 mL) cartridges were used. The cartridges were eluted with 10 mL DCM, then a solution of crude diluted in 1 mL was filtered by gravity. The cartridge was eluted twice with 4 mL of 1.25 N HCl in MeOH and once with 2 mL of 1.25 N HCl in MeOH. The desired fractions were concentrated under nitrogen flow at rt for 2 h, then dried under vacuum at 40° C. overnight. Flash Chromatography: crude product was purified by flash chromatography on silica gel using a gradient of MeOH in DCM from 2% to 10%. The desired fractions were concentrated under nitrogen flow at rt for 2 h and then dried under vacuum at 40° C. overnight. Unless otherwise indicated, Method A was used for LCMS.

The following compounds were prepared following the general procedure provided herein:

### N-((1-((3,4-dimethoxyphenyl)sulfonyl)piperidin-4-yl)methyl)-4-(trifluoromethoxy)benzenesulfonamide (1)

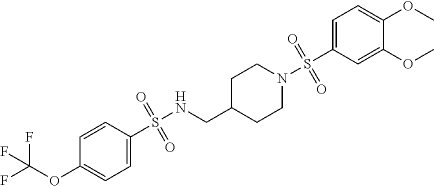 [SMILES: COc1ccc(S(=O)(=O)N2CCC(CNS(=O)(=O)c3ccc(OC(F)(F)F)cc3)CC2)cc1OC]

TABLE 1
Characterization data for 1

| Amount (mg) | Yield (%) | Purity (%) | MS (m/z) | Ion type | tᵣ (min) |
|---|---|---|---|---|---|
| 185.3 | 88.6 | 98.86 | 538.8 | [M + H]⁺ | 2.5 |

Starting material (amine): (1-((3,4-dimethoxyphenyl)sulfonyl)piperidin-4-yl)methanamine.

### N-(2-(piperidin-1-ylsulfonyl)benzyl)-4-(trifluoromethoxy)benzenesulfonamide (2)

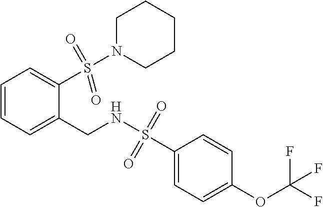 [SMILES: O=S(=O)(NCc1ccccc1S(=O)(=O)N1CCCCC1)c1ccc(OC(F)(F)F)cc1]

TABLE 2
Characterization data for 2

| Amount (mg) | Yield (%) | Purity (%) | MS (m/z) | Ion type | tᵣ (min) |
|---|---|---|---|---|---|
| 154.7 | 80.6 | 95.65 | 478.8 | [M + H]⁺ | 2.64 |

Starting material (amine): (2-(piperidin-1-ylsulfonyl)phenyl)methanamine.

### 2,5-dibromo-N-(2-((4-(trifluoromethoxy)phenyl)sulfonamido)ethyl)benzenesulfonamide (3)

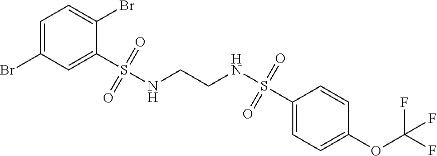 [SMILES: O=S(=O)(NCCNS(=O)(=O)c1cc(Br)ccc1Br)c1ccc(OC(F)(F)F)cc1]

TABLE 3
Characterization data for 3

| Amount (mg) | Yield (%) | Purity (%) | MS (m/z) | Ion type | tᵣ (min) |
|---|---|---|---|---|---|
| 189.4 | 81.5 | 96.13 | 582.7 | [M + H]⁺ | 2.6 |

Starting material (amine): (N-(2-aminoethyl)-2,5-dibromobenzenesulfonamide).

### N-cycloheptyl-N-methyl-2-((4-(trifluoromethoxy)phenyl)sulfonamido)pentanamide (4)

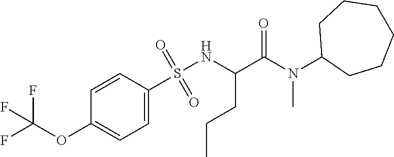 [SMILES: CCCC(NS(=O)(=O)c1ccc(OC(F)(F)F)cc1)C(=O)N(C)C1CCCCCC1]

TABLE 4
Characterization data for 4

| Amount (mg) | Yield (%) | Purity (%) | MS (m/z) | Ion type | tᵣ (min) |
|---|---|---|---|---|---|
| 143 | 82.7 | 100 | 451 | [M + H]⁺ | 2.82 |

Starting material (amine): 2-amino-N-cycloheptyl-N-methylpentanamide.

### Tert-butyl 2-((4-(trifluoromethoxy)phenyl)sulfonamido)hexanoate (5)

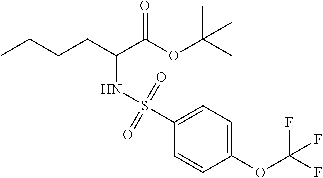 [SMILES: CCCCC(NS(=O)(=O)c1ccc(OC(F)(F)F)cc1)C(=O)OC(C)(C)C]

TABLE 5
Characterization data for 5

| Amount (mg) | Yield (%) | Purity (%) | MS (m/z) | Ion type | tᵣ (min) |
|---|---|---|---|---|---|
| 131 | 79 | 95.2 | 433.9 | [M + Na]⁺ | 2.87 |

Starting material (amine): tert-butyl 2-aminohexanoate.

### Methyl (R)-4-phenyl-3-((4-(trifluoromethoxy)phenyl)sulfonamido)butanoate (6)

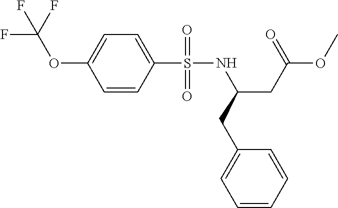 [SMILES: COC(=O)C[C@@H](Cc1ccccc1)NS(=O)(=O)c1ccc(OC(F)(F)F)cc1]

TABLE 6
Characterization data for 6

| Amount (mg) | Yield (%) | Purity (%) | MS (m/z) | Ion type | tᵣ (min) |
|---|---|---|---|---|---|
| 134 | 77.7 | 92.87 | 417.9 | [M + H]⁺ | 2.58 |

Starting material (amine): methyl (R)-3-amino-4-phenylbutanoate.

### Tert-butyl ((4-(trifluoromethoxy)phenyl)sulfonyl)-L-isoleucinate (7)

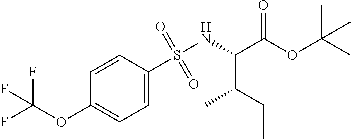 [SMILES: CC[C@H](C)[C@H](NS(=O)(=O)c1ccc(OC(F)(F)F)cc1)C(=O)OC(C)(C)C]

TABLE 7
Characterization data for 7

| Amount (mg) | Yield (%) | Purity (%) | MS (m/z) | Ion type | tᵣ (min) |
|---|---|---|---|---|---|
| 150 | 90.4 | 95.19 | 433.9 | [M + Na]⁺ | 2.85 |

Starting material (amine): tert-butyl L-isoleucinate.

### N-(3,3,3-trifluoro-1-phenylpropyl)-4-(trifluoromethoxy)benzenesulfonamide (8)

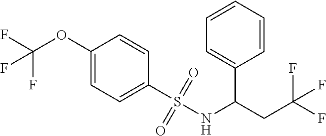 [SMILES: O=S(=O)(NC(CC(F)(F)F)c1ccccc1)c1ccc(OC(F)(F)F)cc1]

TABLE 8
Characterization data for 8

| Amount (mg) | Yield (%) | Purity (%) | MS (m/z) | Ion type | tᵣ (min) |
|---|---|---|---|---|---|
| 84.7 | 51.5 | 96.47 | 413.9 | [M + H]⁺ | 2.64 |

Starting material (amine): 3,3,3-trifluoro-1-phenylpropan-1-amine.

### N-(1-hydroxy-3-(m-tolyl)propan-2-yl)-4-(trifluoromethoxy)benzenesulfonamide (9)

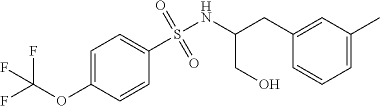 [SMILES: Cc1cccc(CC(CO)NS(=O)(=O)c2ccc(OC(F)(F)F)cc2)c1]

TABLE 9
Characterization data for 9

| Amount (mg) | Yield (%) | Purity (%) | MS (m/z) | Ion type | tᵣ (min) |
|---|---|---|---|---|---|
| 99 | 63.7 | 96.12 | 390 | [M + H]⁺ | 2.43 |

Starting material (amine): 2-amino-3-(m-tolyl)propan-1-ol.

### N-(1-hydroxy-5-methylhexan-3-yl)-4-(trifluoromethoxy)benzenesulfonamide (10)

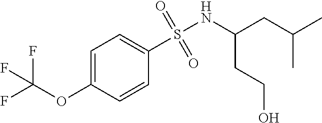 [SMILES: CC(C)CC(CCO)NS(=O)(=O)c1ccc(OC(F)(F)F)cc1]

TABLE 10
Characterization data for 10

| Amount (mg) | Yield (%) | Purity (%) | MS (m/z) | Ion type | tᵣ (min) |
|---|---|---|---|---|---|
| 128 | 89.5 | 95.37 | 356 | [M + H]⁺ | 2.33 |

Starting material (amine): 3-amino-5-methylhexan-1-ol.

### N-(2-(2-hydroxypropoxy)benzyl)-4-(trifluoromethoxy)benzenesulfonamide (11)

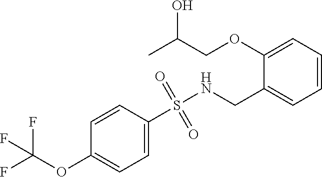 [SMILES: CC(O)COc1ccccc1CNS(=O)(=O)c1ccc(OC(F)(F)F)cc1]

TABLE 11
Characterization data for 11

| Amount (mg) | Yield (%) | Purity (%) | MS (m/z) | Ion type | tᵣ (min) |
|---|---|---|---|---|---|
| 129 | 82.9 | 100 | 405.9 | [M + H]⁺ | 2.43 |

Starting material (amine): 1-(2-(aminomethyl)phenoxy)propan-2-ol.

### (S)—N-(3-hydroxy-1-phenylpropyl)-4-(trifluoromethoxy)benzenesulfonamide (12)

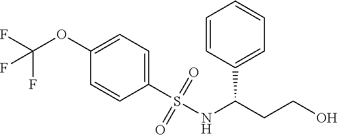 [SMILES: O=S(=O)(N[C@@H](CCO)c1ccccc1)c1ccc(OC(F)(F)F)cc1]

TABLE 12
Characterization data for 12

| Amount (mg) | Yield (%) | Purity (%) | MS (m/z) | Ion type | tᵣ (min) |
|---|---|---|---|---|---|
| 107 | 72.7 | 97.81 | 376 | [M + H]⁺ | 2.28 |

Starting material (amine): (S)-3-amino-3-phenylpropan-1-ol.

### Ethyl 3-(cyclohex-3-en-1-yl)-2-((4-(trifluoromethoxy)phenyl)sulfonamido)propanoate (13)

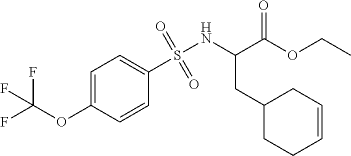 [SMILES: CCOC(=O)C(CC1CC=CCC1)NS(=O)(=O)c1ccc(OC(F)(F)F)cc1]

TABLE 13
Characterization data for 13

| Amount (mg) | Yield (%) | Purity (%) | MS (m/z) | Ion type | tᵣ (min) |
|---|---|---|---|---|---|
| 125.6 | 75 | 96.61 | 421.9 | [M + H]⁺ | 2.75 |

Starting material (amine): ethyl 2-amino-3-(cyclohex-3-en-1-yl)propanoate.

### Ethyl (R)-3-(2-chlorophenyl)-3-((4-(trifluoromethoxy)phenyl)sulfonamido)propanoate (14)

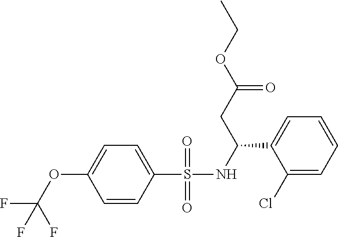 [SMILES: CCOC(=O)C[C@@H](NS(=O)(=O)c1ccc(OC(F)(F)F)cc1)c1ccccc1Cl]

TABLE 14
Characterization data for 14

| Amount (mg) | Yield (%) | Purity (%) | MS (m/z) | Ion type | tᵣ (min) |
|---|---|---|---|---|---|
| 133.1 | 71.1 | 92.67 | 451.8 | [M + H]⁺ | 2.63 |

Starting material (amine): ethyl (R)-3-amino-3-(2-chlorophenyl)propanoate.

### Ethyl 3-(3-chlorophenyl)-2-((4-(trifluoromethoxy)phenyl)sulfonamido)propanoate (15)

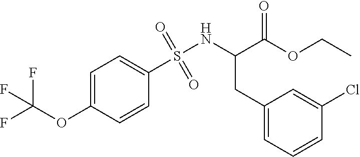 [SMILES: CCOC(=O)C(Cc1cccc(Cl)c1)NS(=O)(=O)c1ccc(OC(F)(F)F)cc1]

TABLE 15
Characterization data for 15

| Amount (mg) | Yield (%) | Purity (%) | MS (m/z) | Ion type | tᵣ (min) |
|---|---|---|---|---|---|
| 136.4 | 76.2 | 96.81 | 451.8 | [M + H]⁺ | 2.71 |

Starting material (amine): ethyl 2-amino-3-(3-chlorophenyl)propanoate.

### Ethyl 2-(3-bromo-4-methoxyphenyl)-2-((4-(trifluoromethoxy)phenyl)sulfonamido)acetate (16)

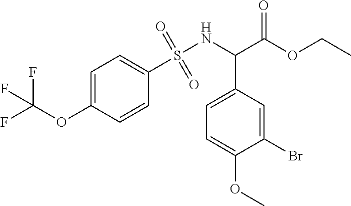 [SMILES: CCOC(=O)C(NS(=O)(=O)c1ccc(OC(F)(F)F)cc1)c1ccc(OC)c(Br)c1]

TABLE 16
Characterization data for 16

| Amount (mg) | Yield (%) | Purity (%) | MS (m/z) | Ion type | tᵣ (min) |
|---|---|---|---|---|---|
| 158.2 | 77.3 | 96.01 | 513.7 | [M + H]⁺ | 2.64 |

Starting material (amine): ethyl 2-amino-2-(3-bromo-4-methoxyphenyl)acetate.

### N-(1-(5-ethylfuran-2-yl)-2,2-dimethylpropyl)-4-(trifluoromethoxy)benzenesulfonamide (17)

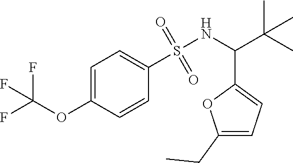 [SMILES: CCc1ccc(C(NS(=O)(=O)c2ccc(OC(F)(F)F)cc2)C(C)(C)C)o1]

TABLE 17
Characterization data for 17

| Amount (mg) | Yield (%) | Purity (%) | MS (m/z) | Ion type^{a} | tᵣ (min) |
|---|---|---|---|---|---|
| 132 | 77.8 | 91.71 | 404 | [M − H]⁻ | 2.91 |

^{a}LCMS Method B; Starting material (amine): 1-(5-ethylfuran-2-y1)-2,2-dimethylpropan-1-amine.

### N-(1-(2-methoxy-5-methylphenyl)-3-methylbutan-2-yl)-4-(trifluoromethoxy)benzenesulfonamide (18)

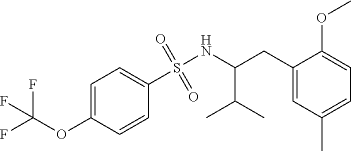 [SMILES: COc1ccc(C)cc1CC(NS(=O)(=O)c1ccc(OC(F)(F)F)cc1)C(C)C]

TABLE 18
Characterization data for 18

| Amount (mg) | Yield (%) | Purity (%) | MS (m/z) | Ion type | tᵣ (min) |
|---|---|---|---|---|---|
| 127.6 | 72.3 | 93.77 | 431.9 | [M + H]⁺ | 2.89 |

Starting material (amine): 1-(2-methoxy-5-methylphenyl)-3-methylbutan-2-amine.

### N-(2-cyclopropyl-1-phenylethyl)-4-(trifluoromethoxy)benzenesulfonamide (19)

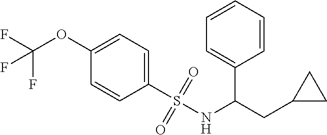 [SMILES: O=S(=O)(NC(CC1CC1)c1ccccc1)c1ccc(OC(F)(F)F)cc1]

TABLE 19
Characterization data for 19

| Amount (mg) | Yield (%) | Purity (%) | MS (m/z) | Ion type^{a} | tᵣ (min) |
|---|---|---|---|---|---|
| 125.7 | 79 | 92.95 | 384 | [M − H]⁻ | 2.66 |

^{a}LCMS Method B; Starting material (amine): 2-cyclopropyl-1-phenylethan-1-amine.

### N-(1-(2-chlorophenyl)-1-methoxypropan-2-yl)-4-(trifluoromethoxy)benzenesulfonamide (20)

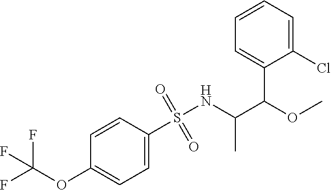 [SMILES: COC(c1ccccc1Cl)C(C)NS(=O)(=O)c1ccc(OC(F)(F)F)cc1]

TABLE 20
Characterization data for 20

| Amount (mg) | Yield (%) | Purity (%) | MS (m/z) | Ion type | tᵣ (min) |
|---|---|---|---|---|---|
| 138 | 78.5 | 92.49 | 423.9 | [M + H]⁺ | 2.74 |

Starting material (amine): 1-(2-chlorophenyl)-1-methoxypropan-2-amine.

### N-(1-(5-fluoro-2-(4-hydroxypiperidin-1-yl)phenyl)ethyl)-4-(trifluoromethoxy)benzenesulfonamide (21)

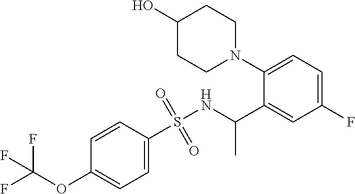 [SMILES: CC(NS(=O)(=O)c1ccc(OC(F)(F)F)cc1)c1cc(F)ccc1N1CCC(O)CC1]

TABLE 21
Characterization data for 21

| Amount (mg) | Yield (%) | Purity (%) | MS (m/z) | Ion type | tᵣ (min) |
|---|---|---|---|---|---|
| 127 | 70.7 | 98.83 | 462.9 | [M + H]⁺ | 2.35 |

Starting material (amine): 1-(2-(1-aminoethyl)-4-fluorophenyl)piperidin-4-ol.

### Methyl 3-(3-bromo-4-methoxyphenyl)-3-((4-(trifluoromethoxy)phenyl)pyrrolidine)propanoate (22)

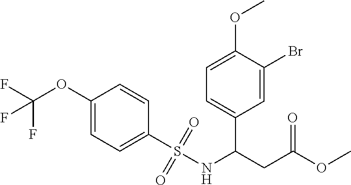 [SMILES: COC(=O)CC(NS(=O)(=O)c1ccc(OC(F)(F)F)cc1)c1ccc(OC)c(Br)c1]

TABLE 22
Characterization data for 22

| Amount (mg) | Yield (%) | Purity (%) | MS (m/z) | Ion type^{a} | tᵣ (min) |
|---|---|---|---|---|---|
| 163 | 82.9 | 100 | 509.8 | [M − H]⁻ | 2.35 |

^{a}LCMS Method B; Starting material (amine): methyl 3-amino-3-(3-bromo-4-methoxyphenyl)propanoate.

### Methyl 2-methyl-3-phenyl-3-((4-(trifluoromethoxy)phenyl)pyrrolidine)propanoate (23)

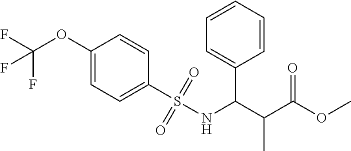 [SMILES: COC(=O)C(C)C(NS(=O)(=O)c1ccc(OC(F)(F)F)cc1)c1ccccc1]

TABLE 23
Characterization data for 23

| Amount (mg) | Yield (%) | Purity (%) | MS (m/z) | Ion type | tᵣ (min) |
|---|---|---|---|---|---|
| 130 | 81.2 | 100 | 417.9 | [M + H]⁺ | 2.55 |

Starting material (amine): methyl 3-amino-2-methyl-3-phenylpropanoate.

### Ethyl 3-(p-tolyl)-3-((4-(trifluoromethoxy)phenyl)pyrrolidine)propanoate (24)

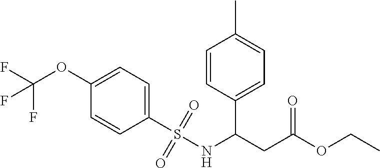 [SMILES: CCOC(=O)CC(NS(=O)(=O)c1ccc(OC(F)(F)F)cc1)c1ccc(C)cc1]

TABLE 24
Characterization data for 24

| Amount (mg) | Yield (%) | Purity (%) | MS (m/z) | Ion type | tᵣ (min) |
|---|---|---|---|---|---|
| 144 | 79.7 | 91.63 | 431.9 | [M + H]⁺ | 2.64 |

Starting material (amine): ethyl 3-amino-3-(p-tolyl)propanoate.

### Ethyl 3-(4-ethoxyphenyl)-3-((4-(trifluoromethoxy)phenyl)pyrrolidine)propanoate (25)

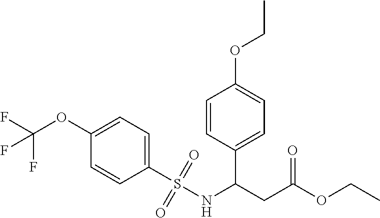 [SMILES: CCOC(=O)CC(NS(=O)(=O)c1ccc(OC(F)(F)F)cc1)c1ccc(OCC)cc1]

TABLE 25
Characterization data for 25

| Amount (mg) | Yield (%) | Purity (%) | MS (m/z) | Ion type^{a} | tᵣ (min) |
|---|---|---|---|---|---|
| 158 | 80.8 | 90.54 | 460 | [M − H]⁻ | 2.51 |

^{a}LCMS Method B; Starting material (amine): ethyl 3-amino-3-(4-ethoxyphenyl)propanoate.

### N-(1-(3-cyanophenyl)-3-methylbutyl)-4-(trifluoromethoxy)benzenesulfonamide (26)

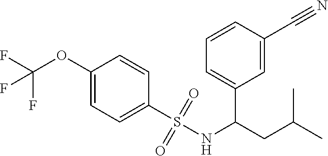 [SMILES: CC(C)CC(NS(=O)(=O)c1ccc(OC(F)(F)F)cc1)c1cccc(C#N)c1]

TABLE 26
Characterization data for 26

| Amount (mg) | Yield (%) | Purity (%) | MS (m/z) | Ion type | tᵣ (min) |
|---|---|---|---|---|---|
| 109.5 | 65.3 | 94.38 | 412.9 | [M + H]⁺ | 2.67 |

Starting material (amine): 3-(1-amino-3-methylbutyl)benzonitrile.

### N-(3-(2,5-dimethoxyphenyl)propyl)-4-(trifluoromethoxy)benzenesulfonamide (27)

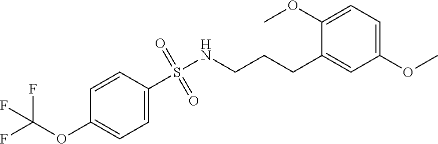 [SMILES: COc1ccc(OC)c(CCCNS(=O)(=O)c2ccc(OC(F)(F)F)cc2)c1]

TABLE 27
Characterization data for 27

| Amount (mg) | Yield (%) | Purity (%) | MS (m/z) | Ion type | tᵣ (min) |
|---|---|---|---|---|---|
| 129.9 | 79.9 | 98.93 | 419.9 | [M + H]⁺ | 2.64 |

Starting material (amine): 3-(2,5-dimethoxyphenyl)propan-1-amine.

### N-(2-(4-ethylphenyl)-2-(pyrrolidine-1-yl)ethyl)-4-(trifluoromethoxy)benzenesulfonamide (28)

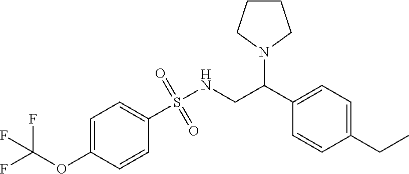 [SMILES: CCc1ccc(C(CNS(=O)(=O)c2ccc(OC(F)(F)F)cc2)N2CCCC2)cc1]

TABLE 28
Characterization data for 28

| Amount (mg) | Yield (%) | Purity (%) | MS (m/z) | Ion type | tᵣ (min) |
|---|---|---|---|---|---|
| 142 | 80.6 | 96.33 | 442.9 | [M + H]⁺ | 2.02 |

Starting material (amine): 2-(4-ethylphenyl)-2-(pyrrolidin-1-yl)ethan-1-amine.

### N-(5-chloro-2-ethoxyphenethyl)-4-(trifluoromethoxy)benzenesulfonamide (29)

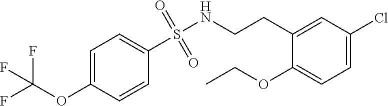 [SMILES: CCOc1ccc(Cl)cc1CCNS(=O)(=O)c1ccc(OC(F)(F)F)cc1]

TABLE 29
Characterization data for 29

| Amount (mg) | Yield (%) | Purity (%) | MS (m/z) | Ion type | tᵣ (min) |
|---|---|---|---|---|---|
| 139.4 | 80.2 | 93.57 | 423.9 | [M + H]⁺ | 2.8 |

Starting material (amine): 2-(5-chloro-2-ethoxyphenyl)ethan-1-amine.

### N-(2-(pyrrolidin-1-yl)benzyl)-4-(trifluoromethoxy)benzenesulfonamide (30)

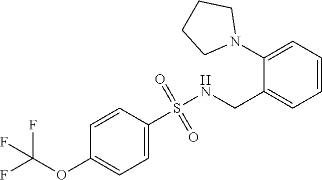 [SMILES: O=S(=O)(NCc1ccccc1N1CCCC1)c1ccc(OC(F)(F)F)cc1]

TABLE 30
Characterization data for 30

| Amount (mg) | Yield (%) | Purity (%) | MS (m/z) | Ion type | tᵣ (min) |
|---|---|---|---|---|---|
| 127.7 | 83.1 | 100 | 400.9 | [M + H]⁺ | 2.07 |

Starting material (amine): (2-(pyrrolidin-1-yl)phenyl)methanamine.

### N-(2-(furan-2-yl)-2-(4-methylpiperidin-1-yl)ethyl)-4-(trifluoromethoxy)benzenesulfonamide (31)

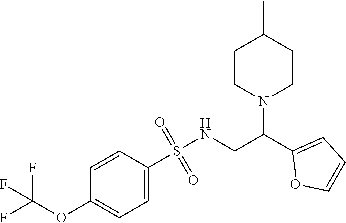 [SMILES: CC1CCN(C(CNS(=O)(=O)c2ccc(OC(F)(F)F)cc2)c2ccco2)CC1]

TABLE 31
Characterization data for 31

| Amount (mg) | Yield (%) | Purity (%) | MS (m/z) | Ion type | tᵣ (min) |
|---|---|---|---|---|---|
| 162 | 97.6 | 100 | 432.9 | [M + H]⁺ | 1.91 |

Starting material (amine): 2-(furan-2-yl)-2-(4-methylpiperidin-1-yl)ethan-1-amine.

### N-(2-morpholino-2-(4-(trifluoromethyl)phenyl)ethyl)-4-(trifluoromethoxy)benzenesulfonamide (32)

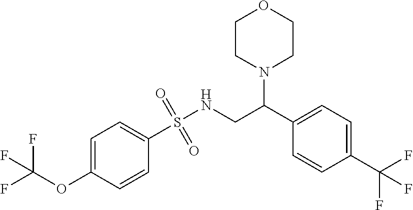 [SMILES: O=S(=O)(NCC(c1ccc(C(F)(F)F)cc1)N1CCOCC1)c1ccc(OC(F)(F)F)cc1]

TABLE 32
Characterization data for 32

| Amount (mg) | Yield (%) | Purity (%) | MS (m/z) | Ion type | tᵣ (min) |
|---|---|---|---|---|---|
| 155.5 | 79.8 | 98.12 | 498.8 | [M + H]⁺ | 2.08 |

Starting material (amine): 2-morpholino-2-(4-(trifluoromethyl)phenyl)ethan-1-amine.

### N-((1-(4-(difluoromethoxy)-3-methoxyphenyl)cyclopentyl)methyl)-4-(trifluoromethoxy)benzenesulfonamide (33)

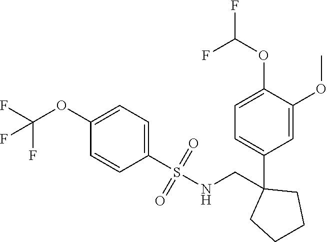 [SMILES: COc1cc(C2(CNS(=O)(=O)c3ccc(OC(F)(F)F)cc3)CCCC2)ccc1OC(F)F]

TABLE 33
Characterization data for 33

| Amount (mg) | Yield (%) | Purity (%) | MS (m/z) | Ion type | tᵣ (min) |
|---|---|---|---|---|---|
| 110.4 | 63.1 | 91.51 | 495.8 | [M + H]⁺ | 2.85 |

Starting material (amine): (1-(4-(difluoromethoxy)-3-methoxyphenyl)cyclopentyl)methanamine.

### N-(cyclopropyl(4-fluoro-3-(trifluoromethyl)phenyl)methyl)-4-(trifluoromethoxy)benzenesulfonamide (34)

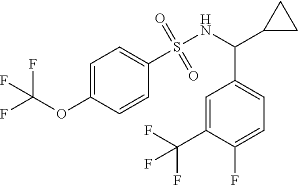 [SMILES: O=S(=O)(NC(c1ccc(F)c(C(F)(F)F)c1)C1CC1)c1ccc(OC(F)(F)F)cc1]

TABLE 34
Characterization data for 34

| Amount (mg) | Yield (%) | Purity (%) | MS (m/z) | Ion type^{a} | tᵣ (min) |
|---|---|---|---|---|---|
| 148 | 81.4 | 96.47 | 456 | [M − H]⁻ | 2.74 |

^{a}LCMS Method B; Starting material (amine): cyclopropyl(4-fluoro-3-(trifluoromethyl)phenyl)methanamine.

### N-(1-(3,4-dichlorophenyl)-3-methoxypropyl)-4-(trifluoromethoxy)benzenesulfonamide (35)

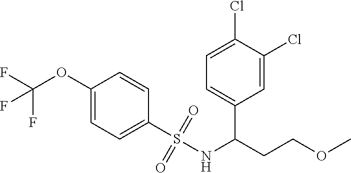 [SMILES: COCCC(NS(=O)(=O)c1ccc(OC(F)(F)F)cc1)c1ccc(Cl)c(Cl)c1]

TABLE 35
Characterization data for 35

| Amount (mg) | Yield (%) | Purity (%) | MS (m/z) | Ion type | tᵣ (min) |
|---|---|---|---|---|---|
| 153 | 86 | 98.83 | 457.8 | [M + H]⁺ | 2.71 |

Starting material (amine): 1-(3,4-dichlorophenyl)-3-methoxypropan-1-amine.

### N-(cyclohexyl(3-(trifluoromethyl)phenyl)methyl)-4-(trifluoromethoxy)benzenesulfonamide (36)

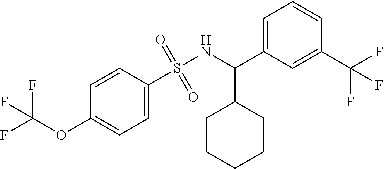 [SMILES: O=S(=O)(NC(c1cccc(C(F)(F)F)c1)C1CCCCC1)c1ccc(OC(F)(F)F)cc1]

TABLE 36
Characterization data for 36

| Amount (mg) | Yield (%) | Purity (%) | MS (m/z) | Ion type | tᵣ (min) |
|---|---|---|---|---|---|
| 168 | 88.8 | 97.64 | 481.8 | [M + H]⁺ | 2.99 |

Starting material (amine): cyclohexyl(3-(trifluoromethyl)phenyl)methanamine.

### Methyl 3-cyclohexyl-3-((4-(trifluoromethoxy)phenyl)sulfonamido)propanoate (37)

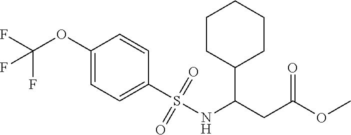 [SMILES: COC(=O)CC(NS(=O)(=O)c1ccc(OC(F)(F)F)cc1)C1CCCCC1]

TABLE 37
Characterization data for 37

| Amount (mg) | Yield (%) | Purity (%) | MS (m/z) | Ion type | tᵣ (min) |
|---|---|---|---|---|---|
| 89 | 52.4 | 92.49 | 409.9 | [M + H]⁺ | 2.65 |

Starting material (amine): methyl 3-amino-3-cyclohexylpropanoate.

### Methyl 3-(3-bromophenyl)-3-((4-(trifluoromethoxy)phenyl)sulfonamido)propanoate (38)

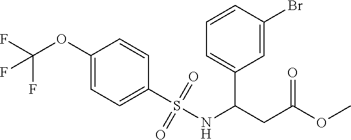 [SMILES: COC(=O)CC(NS(=O)(=O)c1ccc(OC(F)(F)F)cc1)c1cccc(Br)c1]

TABLE 38
Characterization data for 38

| Amount (mg) | Yield (%) | Purity (%) | MS (m/z) | Ion type | tᵣ (min) |
|---|---|---|---|---|---|
| 149 | 80.5 | 100 | 483.7 | [M + H]⁺ | 2.59 |

Starting material (amine): methyl 3-amino-3-(3-bromophenyl)propanoate.

### Methyl (S)-3-(3,5-dichlorophenyl)-3-((4-(trifluoromethoxy)phenyl)sulfonamido)propanoate (39)

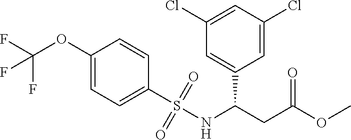 [SMILES: COC(=O)C[C@H](NS(=O)(=O)c1ccc(OC(F)(F)F)cc1)c1cc(Cl)cc(Cl)c1]

TABLE 39
Characterization data for 39

| Amount (mg) | Yield (%) | Purity (%) | MS (m/z) | Ion type | tᵣ (min) |
|---|---|---|---|---|---|
| 121 | 66.8 | 100 | 471.7 | [M + H]⁺ | 2.68 |

Starting material (amine): methyl (S)-3-amino-3-(3,5-dichlorophenyl)propanoate.

### Methyl (S)-3-(3,4-dichlorophenyl)-2-((4-(trifluoromethoxy)phenyl)sulfonamido)propanoate (40)

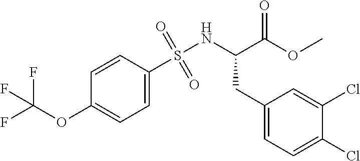 [SMILES: COC(=O)[C@H](Cc1ccc(Cl)c(Cl)c1)NS(=O)(=O)c1ccc(OC(F)(F)F)cc1]

TABLE 40
Characterization data for 40

| Amount (mg) | Yield (%) | Purity (%) | MS (m/z) | Ion type | tᵣ (min) |
|---|---|---|---|---|---|
| 109 | 59.3 | 98.58 | 471.8 | [M + H]⁺ | 2.71 |

Starting material (amine): methyl (S)-2-amino-3-(3,4-dichlorophenyl)propanoate.

### N-((3-chlorophenyl)(cyclopentyl)methyl)-4-(trifluoromethoxy)benzenesulfonamide (41)

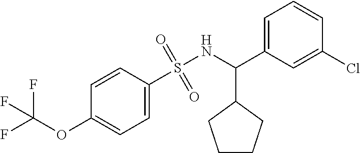 [SMILES: O=S(=O)(NC(c1cccc(Cl)c1)C1CCCC1)c1ccc(OC(F)(F)F)cc1]

TABLE 41
Characterization data for 41

| Amount (mg) | Yield (%) | Purity (%) | MS (m/z) | Ion type | tᵣ (min) |
|---|---|---|---|---|---|
| 76.7 | 45.4 | 98.44 | 433.8 | [M + H]⁺ | 2.9 |

Starting material (amine): (3-chlorophenyl)(cyclopentyl)methanamine.

### Ethyl 3-(2,5-dichlorothiophen-3-yl)-2-((4-(trifluoromethoxy)phenyl)sulfonamido)propanoate (42)

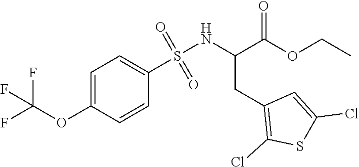 [SMILES: CCOC(=O)C(Cc1cc(Cl)sc1Cl)NS(=O)(=O)c1ccc(OC(F)(F)F)cc1]

TABLE 42
Characterization data for 42

| Amount (mg) | Yield (%) | Purity (%) | MS (m/z) | Ion type | tᵣ (min) |
|---|---|---|---|---|---|
| 81.6 | 42.6 | 98.67 | 491.7 | [M + H]⁺ | 2.85 |

Starting material (amine): ethyl 2-amino-3-(2,5-dichlorothiophen-3-yl)propanoate.

### Ethyl (R)-3-(4-methoxyphenyl)-2-((4-(trifluoromethoxy)phenyl)sulfonamido)propanoate (43)

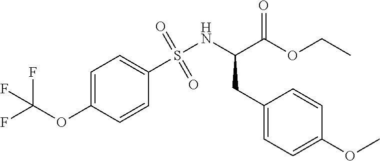 [SMILES: CCOC(=O)[C@@H](Cc1ccc(OC)cc1)NS(=O)(=O)c1ccc(OC(F)(F)F)cc1]

TABLE 43
Characterization data for 43

| Amount (mg) | Yield (%) | Purity (%) | MS (m/z) | Ion type | tᵣ (min) |
|---|---|---|---|---|---|
| 66.1 | 38 | 98.64 | 447.9 | [M + H]⁺ | 2.61 |

Starting material (amine): ethyl (R)-2-amino-3-(4-methoxyphenyl)propanoate.

### N-(1-(3-bromophenyl)-4,4,4-trifluorobutan-2-yl)-4-(trifluoromethoxy)benzenesulfonamide (44)

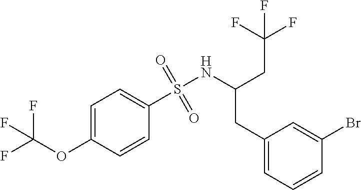 [SMILES: O=S(=O)(NC(Cc1cccc(Br)c1)CC(F)(F)F)c1ccc(OC(F)(F)F)cc1]

TABLE 44
Characterization data for 44

| Amount (mg) | Yield (%) | Purity (%) | MS (m/z) | Ion type | tᵣ (min) |
|---|---|---|---|---|---|
| 75.5 | 37.4 | 96.29 | 507.6 | [M + H]⁺ | 2.81 |

Starting material (amine): 1-(3-bromophenyl)-4,4,4-trifluorobutan-2-amine.

### Ethyl 3-(2-chlorophenyl)-2-((4-(trifluoromethoxy)phenyl)sulfonamido)propanoate (45)

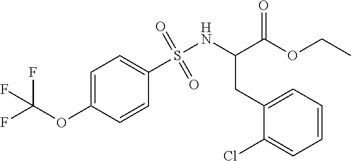 [SMILES: CCOC(=O)C(Cc1ccccc1Cl)NS(=O)(=O)c1ccc(OC(F)(F)F)cc1]

TABLE 45
Characterization data for 45

| Amount (mg) | Yield (%) | Purity (%) | MS (m/z) | Ion type | tᵣ (min) |
|---|---|---|---|---|---|
| 116 | 65.8 | 98.28 | 451.8 | [M + H]⁺ | 2.68 |

Starting material (amine): ethyl 2-amino-3-(2-chlorophenyl)propanoate.

### Ethyl 3-(3-bromophenyl)-3-((4-(trifluoromethoxy)phenyl)sulfonamido)propanoate (46)

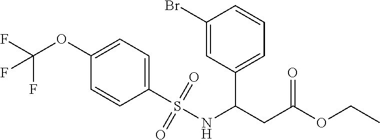 [SMILES: CCOC(=O)CC(NS(=O)(=O)c1ccc(OC(F)(F)F)cc1)c1cccc(Br)c1]

TABLE 46
Characterization data for 46

| Amount (mg) | Yield (%) | Purity (%) | MS (m/z) | Ion type | tᵣ (min) |
|---|---|---|---|---|---|
| 139 | 71.8 | 98.35 | 497.7 | [M + H]⁺ | 2.66 |

Starting material (amine): ethyl 3-amino-3-(3-bromophenyl)propanoate.

### Ethyl 4-methyl-2-((4-(trifluoromethoxy)phenyl)sulfonamido)hexanoate (47)

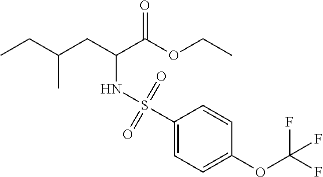 [SMILES: CCOC(=O)C(CC(C)CC)NS(=O)(=O)c1ccc(OC(F)(F)F)cc1]

TABLE 47
Characterization data for 47

| Amount (mg) | Yield (%) | Purity (%) | MS (m/z) | Ion type | tᵣ (min) |
|---|---|---|---|---|---|
| 85.1 | 54.6 | 97.78 | 397.9 | [M + H]⁺ | 2.73 |

Starting material (amine): ethyl 2-amino-4-methylhexanoate.

### N-(2-bromobenzyl)-N-methyl-3-((4-(trifluoromethoxy)phenyl)sulfonamido)propenamide (48)

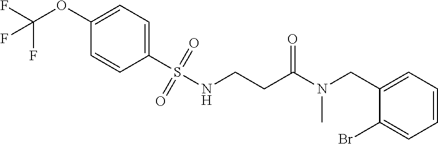 [SMILES: CN(Cc1ccccc1Br)C(=O)CCNS(=O)(=O)c1ccc(OC(F)(F)F)cc1]

TABLE 48
Characterization data for 48

| Amount (mg) | Yield (%) | Purity (%) | MS (m/z) | Ion type | tᵣ (min) |
|---|---|---|---|---|---|
| 85.2 | 42.4 | 94.67 | 494.7 | [M + H]⁺ | 2.55 |

Starting material (amine): 3-amino-N-(2-bromobenzyl)-N-methylpropanamide.

### N-((2-phenylcyclohexyl)methyl)-4-(trifluoromethoxy)benzenesulfonamide (49)

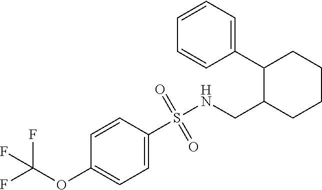 [SMILES: O=S(=O)(NCC1CCCCC1c1ccccc1)c1ccc(OC(F)(F)F)cc1]

TABLE 49
Characterization data for 49

| Amount (mg) | Yield (%) | Purity (%) | MS (m/z) | Ion type | tᵣ (min) |
|---|---|---|---|---|---|
| 148.8 | 91.3 | 97.31 | 413.9 | [M + H]⁺ | 2.87 |

Starting material (amine): (2-phenylcyclohexyl)methanamine.

### N-(4,4,4-trifluoro-3-hydroxy-3-(trifluoromethyl)butyl)-4-(trifluoromethoxy)benzenesulfonamide (50)

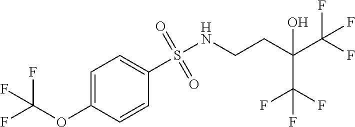 [SMILES: O=S(=O)(NCCC(O)(C(F)(F)F)C(F)(F)F)c1ccc(OC(F)(F)F)cc1]

TABLE 50
Characterization data for 50

| Amount (mg) | Yield (%) | Purity (%) | MS (m/z) | Ion type | tᵣ (min) |
|---|---|---|---|---|---|
| 156 | 88.1 | 94.27 | 435.8 | [M + H]⁺ | 2.47 |

Starting material (amine): 4-amino-1,1,1-trifluoro-2-(trifluoromethyl)butan-2-ol.

### N-(4,4,4-trifluoro-3-phenylbutyl)-4-(trifluoromethoxy)benzenesulfonamide (51)

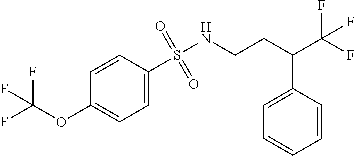 [SMILES: O=S(=O)(NCCC(c1ccccc1)C(F)(F)F)c1ccc(OC(F)(F)F)cc1]

TABLE 51
Characterization data for 51

| Amount (mg) | Yield (%) | Purity (%) | MS (m/z) | Ion type | tᵣ (min) |
|---|---|---|---|---|---|
| 160.5 | 96.7 | 98.81 | 427.9 | [M + H]⁺ | 2.7 |

Starting material (amine): 4,4,4-trifluoro-3-phenylbutan-1-amine.

### N-(3-(1-cyanopropoxy)benzyl)-4-(trifluoromethoxy)benzenesulfonamide (52)

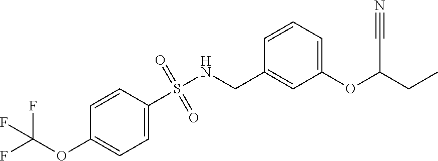 [SMILES: CCC(C#N)Oc1cccc(CNS(=O)(=O)c2ccc(OC(F)(F)F)cc2)c1]

TABLE 52
Characterization data for 52

| Amount (mg) | Yield (%) | Purity (%) | MS (m/z) | Ion type | tᵣ (min) |
|---|---|---|---|---|---|
| 150.5 | 94.5 | 100 | 414.9 | [M + H]⁺ | 2.6 |

Starting material (amine): 2-(3-(aminomethyl)phenoxy)butanenitrile.

### N-((1-(2-methoxyethyl)cyclohexyl)methyl)-4-(trifluoromethoxy)benzenesulfonamide (53)

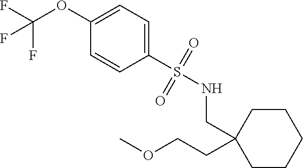 [SMILES: COCCC1(CNS(=O)(=O)c2ccc(OC(F)(F)F)cc2)CCCCC1]

TABLE 53
Characterization data for 53

| Amount (mg) | Yield (%) | Purity (%) | MS (m/z) | Ion type | tᵣ (min) |
|---|---|---|---|---|---|
| 130.4 | 84.1 | 97.8 | 396 | [M + H]⁺ | 2.77 |

Starting material (amine): (1-(2-methoxyethyl)cyclohexyl)methanamine.

### N-(2-(butyl(3-chlorobenzyl)amino)ethyl)-4-(trifluoromethoxy)benzenesulfonamide (54)

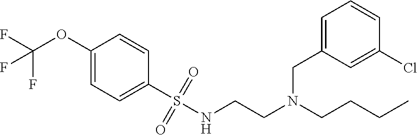 [SMILES: CCCCN(CCNS(=O)(=O)c1ccc(OC(F)(F)F)cc1)Cc1cccc(Cl)c1]

TABLE 54
Characterization data for 54

| Amount (mg) | Yield (%) | Purity (%) | MS (m/z) | Ion type | tᵣ (min) |
|---|---|---|---|---|---|
| 179.3 | 97.4 | 96.88 | 464.9 | [M + H]⁺ | 2.1 |

Starting material (amine): N-butyl-N-(3-chlorobenzyl)ethane-1,2-diamine.

### N-(2-(isopentyloxy)phenethyl)-4-(trifluoromethoxy)benzenesulfonamide (55)

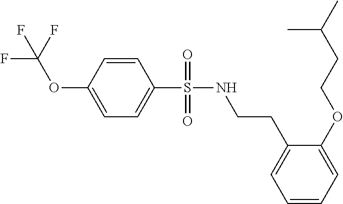 [SMILES: CC(C)CCOc1ccccc1CCNS(=O)(=O)c1ccc(OC(F)(F)F)cc1]

TABLE 55
Characterization data for 55

| Amount (mg) | Yield (%) | Purity (%) | MS (m/z) | Ion type | tᵣ (min) |
|---|---|---|---|---|---|
| 112.2 | 65 | 95.95 | 431.9 | [M + H]⁺ | 2.96 |

Starting material (amine): 2-(2-(isopentyloxy)phenyl)ethan-1-amine.

### N-((1-(2,6-dichlorophenyl)cyclobutyl)methyl)-4-(trifluoromethoxy)benzenesulfonamide (56)

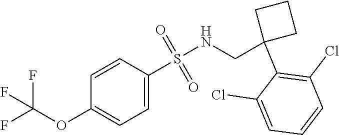 [SMILES: O=S(=O)(NCC1(c2c(Cl)cccc2Cl)CCC1)c1ccc(OC(F)(F)F)cc1]

TABLE 56
Characterization data for 56

| Amount (mg) | Yield (%) | Purity (%) | MS (m/z) | Ion type | tᵣ (min) |
|---|---|---|---|---|---|
| 138.7 | 76.2 | 95.79 | 453.8 | [M + H]⁺ | 2.91 |

Starting material (amine): (1-(2,6-dichlorophenyl)cyclobutyl)methanamine.

### 4-(trifluoromethoxy)-N-((1-(3-(trifluoromethyl)phenyl)cyclobutyl)methyl)benzenesulfonamide (57)

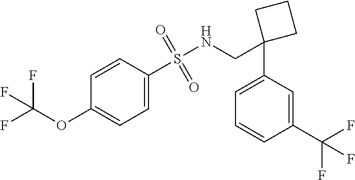 [SMILES: O=S(=O)(NCC1(c2cccc(C(F)(F)F)c2)CCC1)c1ccc(OC(F)(F)F)cc1]

TABLE 57
Characterization data for 57

| Amount (mg) | Yield (%) | Purity (%) | MS (m/z) | Ion type | tᵣ (min) |
|---|---|---|---|---|---|
| 130.5 | 75 | 100 | 453.9 | [M + H]⁺ | 2.88 |

Starting material (amine): (1-(3-(trifluoromethyl)phenyl)cyclobutyl)methanamine.

### N-(2-(isopropyl(methyl)amino)benzyl)-4-(trifluoromethoxy)benzenesulfonamide (58)

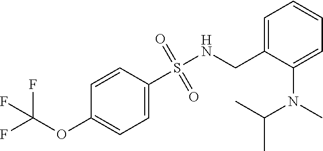 [SMILES: CC(C)N(C)c1ccccc1CNS(=O)(=O)c1ccc(OC(F)(F)F)cc1]

TABLE 58
Characterization data for 58

| Amount (mg) | Yield (%) | Purity (%) | MS (m/z) | Ion type | tᵣ (min) |
|---|---|---|---|---|---|
| 96.2 | 62.3 | 100 | 403 | [M + H]⁺ | 2.06 |

Starting material (amine): 2-(aminomethyl)-N-isopropyl-N-methylaniline.

### N-(2-((cyclohexyloxy)methyl)benzyl)-4-(trifluoromethoxy)benzenesulfonamide (59)

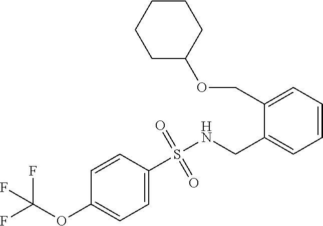 [SMILES: O=S(=O)(NCc1ccccc1COC1CCCCC1)c1ccc(OC(F)(F)F)cc1]

TABLE 59
Characterization data for 59

| Amount (mg) | Yield (%) | Purity (%) | MS (m/z) | Ion type | tᵣ (min) |
|---|---|---|---|---|---|
| 102.5 | 60.2 | 100 | 443.9 | [M + H]⁺ | 2.9 |

Starting material (amine): (2-((cyclohexyloxy)methyl)phenyl)methanamine.

### N-((1-(2,4-dimethylphenyl)cyclopropyl)methyl)-4-(trifluoromethoxy)benzenesulfonamide (60)

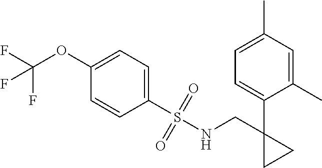 [SMILES: Cc1ccc(C2(CNS(=O)(=O)c3ccc(OC(F)(F)F)cc3)CC2)c(C)c1]

TABLE 60
Characterization data for 60

| Amount (mg) | Yield (%) | Purity (%) | MS (m/z) | Ion type | tᵣ (min) |
|---|---|---|---|---|---|
| 107.6 | 68.4 | 97.4 | 399.9 | [M + H]⁺ | 2.81 |

Starting material (amine): (1-(2,4-dimethylphenyl)cyclopropyl)methanamine.

### N-((1-(thiophen-2-yl)cyclopentyl)methyl)-4-(trifluoromethoxy)benzenesulfonamide (61)

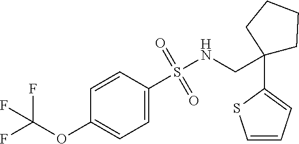 [SMILES: O=S(=O)(NCC1(c2cccs2)CCCC1)c1ccc(OC(F)(F)F)cc1]

TABLE 61
Characterization data for 61

| Amount (mg) | Yield (%) | Purity (%) | MS (m/z) | Ion type | tᵣ (min) |
|---|---|---|---|---|---|
| 91.8 | 59 | 100 | 405.9 | [M + H]⁺ | 2.8 |

Starting material (amine): (1-(thiophen-2-yl)cyclopentyl)methanamine.

### N-(5-chloro-2-(difluoromethoxy)benzyl)-4-(trifluoromethoxy)benzenesulfonamide (62)

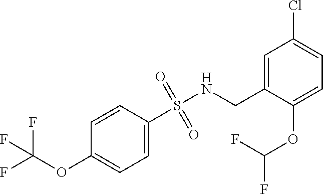 [SMILES: O=S(=O)(NCc1cc(Cl)ccc1OC(F)F)c1ccc(OC(F)(F)F)cc1]

TABLE 62
Characterization data for 62

| Amount (mg) | Yield (%) | Purity (%) | MS (m/z) | Ion type | tᵣ (min) |
|---|---|---|---|---|---|
| 103.5 | 60.2 | 96.36 | 431.8 | [M + H]⁺ | 2.64 |

Starting material (amine): (5-chloro-2-(difluoromethoxy)phenyl)methanamine.

### N-(2-(diisobutylamino)ethyl)-4-(trifluoromethoxy)benzenesulfonamide (63)

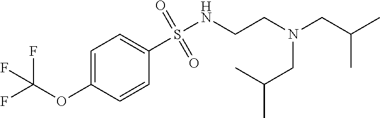 [SMILES: CC(C)CN(CCNS(=O)(=O)c1ccc(OC(F)(F)F)cc1)CC(C)C]

TABLE 63
Characterization data for 63

| Amount (mg) | Yield (%) | Purity (%) | MS (m/z) | Ion type | tᵣ (min) |
|---|---|---|---|---|---|
| 116.7 | 76.7 | 100 | 397 | [M + H]⁺ | 1.93 |

Starting material (amine): N,N-diisobutylethane-1,2-diamine.

### N-(3-(benzyl(butyl)amino)propyl)-4-(trifluoromethoxy)benzenesulfonamide (64)

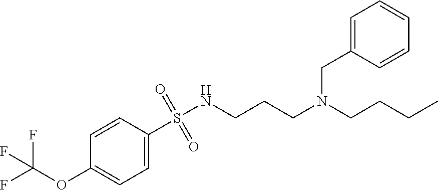 [SMILES: CCCCN(CCCNS(=O)(=O)c1ccc(OC(F)(F)F)cc1)Cc1ccccc1]

TABLE 64
Characterization data for 64

| Amount (mg) | Yield (%) | Purity (%) | MS (m/z) | Ion type | tᵣ (min) |
|---|---|---|---|---|---|
| 119.1 | 69.8 | 100 | 445 | [M + H]⁺ | 2.02 |

Starting material (amine): N¹-benzyl-N¹-butylpropane-1,3-diamine.

### Tert-butyl 4,4-difluoro-2-(((4-(trifluoromethoxy)phenyl)sulfonamido)methyl)pyrrolidine-1-carboxylate (65)

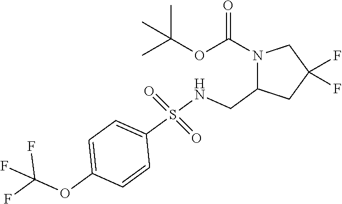 [SMILES: CC(C)(C)OC(=O)N1CC(F)(F)CC1CNS(=O)(=O)c1ccc(OC(F)(F)F)cc1]

TABLE 65
Characterization data for 65

| Amount (mg) | Yield (%) | Purity (%) | MS (m/z) | Ion type | tᵣ (min) |
|---|---|---|---|---|---|
| 164 | 88.1 | 94.93 | 482.8 | [M + Na]⁺ | 2.65 |

Starting material (amine): tert-butyl 2-(aminomethyl)-4,4-difluoropyrrolidine-1-carboxylate.

### (S)—N-(1-(2,5-difluorophenyl)propyl)-4-(trifluoromethoxy)benzenesulfonamide (66)

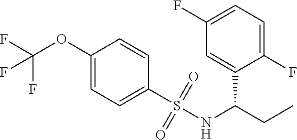 [SMILES: CC[C@H](NS(=O)(=O)c1ccc(OC(F)(F)F)cc1)c1cc(F)ccc1F]

TABLE 66
Characterization data for 66

| Amount (mg) | Yield (%) | Purity (%) | MS (m/z) | Ion type | tᵣ (min) |
|---|---|---|---|---|---|
| 123.6 | 75.8 | 93.07 | 395.9 | [M + H]⁺ | 2.62 |

Starting material (amine): (S)-1-(2,5-difluorophenyl)propan-1-amine.

### N-(1-(2,6-dichlorophenyl)propan-2-yl)-4-(trifluoromethoxy)benzenesulfonamide (67)

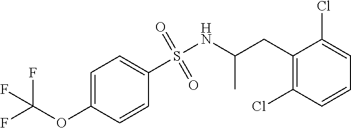 [SMILES: CC(Cc1c(Cl)cccc1Cl)NS(=O)(=O)c1ccc(OC(F)(F)F)cc1]

TABLE 67
Characterization data for 67

| Amount (mg) | Yield (%) | Purity (%) | MS (m/z) | Ion type | tᵣ (min) |
|---|---|---|---|---|---|
| 135 | 77.8 | 94.68 | 427.8 | [M + H]⁺ | 2.78 |

Starting material (amine): 1-(2,6-dichlorophenyl)propan-2-amine.

### Ethyl 3-(4-isopropoxy-3-methoxyphenyl)-3-((4-(trifluoromethoxy)phenyl)sulfonamido)propanoate (68)

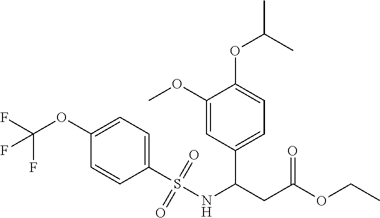 [SMILES: CCOC(=O)CC(NS(=O)(=O)c1ccc(OC(F)(F)F)cc1)c1ccc(OC(C)C)c(OC)c1]

TABLE 68
Characterization data for 68

| Amount (mg) | Yield (%) | Purity (%) | MS (m/z) | Ion type^{a} | tᵣ (min) |
|---|---|---|---|---|---|
| 109 | 46.2 | 100 | 504 | [M − H]⁻ | 2.56 |

^{a}LCMS Method B; Starting material (amine): ethyl 3-amino-3-(4-isopropoxy-3-methoxyphenyl)propanoate.

### N-(cyclohexyl(3,5-dichlorophenyl)methyl)-4-(trifluoromethoxy)benzenesulfonamide (69)

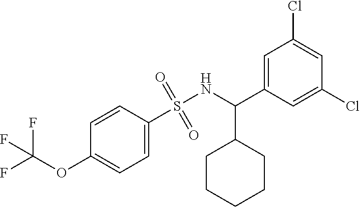 [SMILES: O=S(=O)(NC(c1cc(Cl)cc(Cl)c1)C1CCCCC1)c1ccc(OC(F)(F)F)cc1]

TABLE 69
Characterization data for 69

| Amount (mg) | Yield (%) | Purity (%) | MS (m/z) | Ion type | tᵣ (min) |
|---|---|---|---|---|---|
| 109.8 | 56.6 | 95.35 | 481.9 | [M + H]⁺ | 3.07 |

Starting material (amine): cyclohexyl(3,5-dichlorophenyl)methanamine.

### N-(1-(1-(4-bromophenyl)cyclopropyl)ethyl)-4-(trifluoromethoxy)benzenesulfonamide (70)

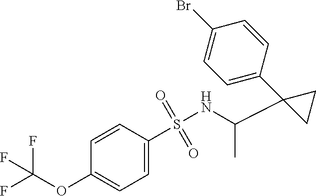 [SMILES: CC(NS(=O)(=O)c1ccc(OC(F)(F)F)cc1)C1(c2ccc(Br)cc2)CC1]

TABLE 70
Characterization data for 70

| Amount (mg) | Yield (%) | Purity (%) | MS (m/z) | Ion type^{a} | tᵣ (min) |
|---|---|---|---|---|---|
| 124.9 | 63.5 | 90.59 | 461.9 | [M − H]⁻ | 2.95 |

^{a}LCMS Method B; Starting material (amine): 1-(1-(4-bromophenyl)cyclopropyl)ethan-1-amine.

^{a}LCMS Method B; Starting material (amine): 1-(1-(4-bromophenyl)cyclopropyl)ethan-1-amine.

### 4-(trifluoromethoxy)-N-(1-(4-(trifluoromethyl)phenyl)pent-4-en-1-yl)benzenesulfonamide (71)

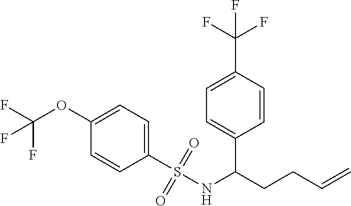 [SMILES: C=CCCC(NS(=O)(=O)c1ccc(OC(F)(F)F)cc1)c1ccc(C(F)(F)F)cc1]

TABLE 71
Characterization data for 71

| Amount (mg) | Yield (%) | Purity (%) | MS (m/z) | Ion type | tᵣ (min) |
|---|---|---|---|---|---|
| 166.7 | 88 | 91.8 | 453.9 | [M + H]⁺ | 2.84 |
| ¹H-NMR (600 | 8.55 (br d, J = 8.9 Hz, 1 H), 7.60 (d, J = 9.0 Hz, 2 H), 7.42 (d, J = 8.1 Hz, 2 | | | | |
| MHz, | H), 7.25-7.29 (m, 4 H), 5.72 (dd, J = 17.0, 10.4 Hz, 1 H), 4.90-4.96 (m, 2 | | | | |
| DMSO-d₆, | H), 4.33 (br d, J = 6.6 Hz, 1 H), 1.99 (br d, J = 6.7 Hz, 1 H), 1.84-1.93 (m, | | | | |
| 300K) δ | 1 H), 1.72-1.79 (m, 1 H), 1.59-1.68 (m, 1 H) | | | | |
| (ppm) | | | | | |

Starting material (amine): 1-(4-(trifluoromethyl)phenyl)pent-4-en-1-amine.

### N-(1-(3-bromo-5-fluorophenyl)-2-cyclopropylethyl)-4-(trifluoromethoxy)benzenesulfonamide (72)

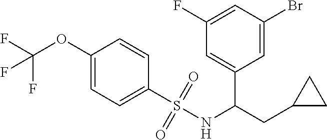 [SMILES: O=S(=O)(NC(CC1CC1)c1cc(F)cc(Br)c1)c1ccc(OC(F)(F)F)cc1]

TABLE 72
Characterization data for 72

| Amount (mg) | Yield (%) | Purity (%) | MS (m/z) | Ion type | tᵣ (min) |
|---|---|---|---|---|---|
| 106.2 | 54.8 | 95.5 | 481.7 | [M + H]⁺ | 2.83 |

Starting material (amine): 1-(3-bromo-5-fluorophenyl)-2-cyclopropylethan-1-amine.

### (R)—N-(1-(benzo[d][1,3]dioxol-5-yl)butyl)-4-(trifluoromethoxy)benzenesulfonamide (73)

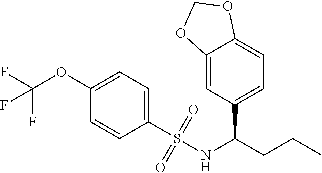 [SMILES: CCC[C@@H](NS(=O)(=O)c1ccc(OC(F)(F)F)cc1)c1ccc2c(c1)OCO2]

TABLE 73
Characterization data for 73

| Amount (mg) | Yield (%) | Purity (%) | MS (m/z) | Ion type^{a} | tᵣ (min) |
|---|---|---|---|---|---|
| 140 | 82.8 | 94.77 | 416 | [M − H]⁻ | 2.77 |

^{a}LCMS Method B; Starting material (amine): (R)-1-(benzo[d][1,3]dioxol-5-yl)butan-1-amine.

### N-(2-isobutoxy-4-methylbenzyl)-4-(trifluoromethoxy)benzenesulfonamide (74)

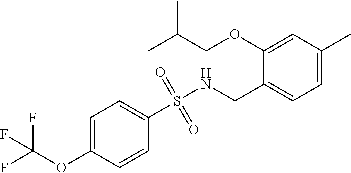 [SMILES: Cc1ccc(CNS(=O)(=O)c2ccc(OC(F)(F)F)cc2)c(OCC(C)C)c1]

TABLE 74
Characterization data for 74

| Amount (mg) | Yield (%) | Purity (%) | MS (m/z) | Ion type^{a} | tᵣ (min) |
|---|---|---|---|---|---|
| 139.4 | 81.7 | 93.86 | 416 | [M − H]⁻ | 3.18 |

^{a}LCMS Method B; Starting material (amine): (2-isobutoxy-4-methylphenyl)methanamine.

### N-(2-(cyclopropylmethoxy)-4-methylbenzyl)-4-(trifluoromethoxy)benzenesulfonamide (75)

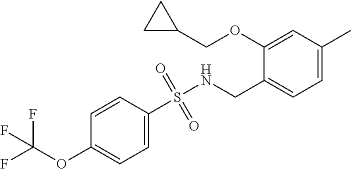 [SMILES: Cc1ccc(CNS(=O)(=O)c2ccc(OC(F)(F)F)cc2)c(OCC2CC2)c1]

TABLE 75
Characterization data for 75

| Amount (mg) | Yield (%) | Purity (%) | MS (m/z) | Ion type^{a} | tᵣ (min) |
|---|---|---|---|---|---|
| 155.3 | 92.4 | 94.84 | 414 | [M − H]⁻ | 3.03 |

^{a}LCMS Method B; Starting material (amine): (2-(cyclopropylmethoxy)-4-methylphenyl)methanamine.

### N-(2-ethoxybenzyl)-4-(trifluoromethoxy)benzenesulfonamide (76)

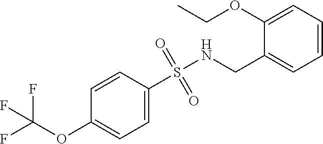 [SMILES: CCOc1ccccc1CNS(=O)(=O)c1ccc(OC(F)(F)F)cc1]

TABLE 76
Characterization data for 76

| Amount (mg) | Yield (%) | Purity (%) | MS (m/z) | Ion type^{a} | tᵣ (min) |
|---|---|---|---|---|---|
| 127.8 | 88.7 | 100 | 374 | [M − H]⁻ | 2.73 |

^{a}LCMS Method B; Starting material (amine): (2-ethoxyphenyl)methanamine.

### Tert-butyl 2,2-dimethyl-6-(((4-(trifluoromethoxy)phenyl)sulfonamido)methyl)morpholine-4-carboxylate (77)

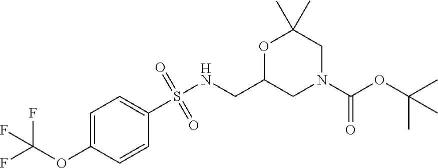 [SMILES: CC(C)(C)OC(=O)N1CC(CNS(=O)(=O)c2ccc(OC(F)(F)F)cc2)OC(C)(C)C1]

TABLE 77
Characterization data for 77

| Amount (mg) | Yield (%) | Purity (%) | MS (m/z) | Ion type | tᵣ (min) |
|---|---|---|---|---|---|
| 80 | 40.1 | 90 | 468.9 | [M + H]⁺ | 2.65 |

Starting material (amine): tert-butyl 6-(aminomethyl)-2,2-dimethylmorpholine-4-carboxylate.

### N-(2-(tert-butoxy)benzyl)-4-(trifluoromethoxy)benzenesulfonamide (78)

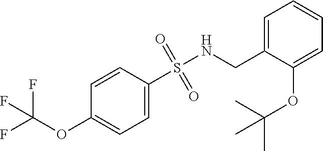 [SMILES: CC(C)(C)Oc1ccccc1CNS(=O)(=O)c1ccc(OC(F)(F)F)cc1]

TABLE 78
Characterization data for 78

| Amount (mg) | Yield (%) | Purity (%) | MS (m/z) | Ion type | tᵣ (min) |
|---|---|---|---|---|---|
| 86.1 | 54.2 | 97.51 | 425.9 | [M + Na]⁺ | 2.73 |

Starting material (amine): (2-(tert-butoxy)phenyl)methanamine.

### N-(3-((furan-2-ylmethyl)((tetrahydrofuran-2-yl)methyl)amino)propyl)-4-(trifluoromethoxy)benzenesulfonamide (79)

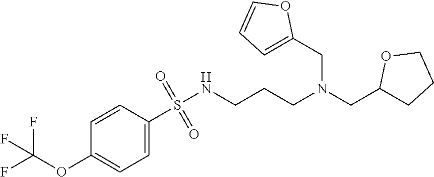 [SMILES: O=S(=O)(NCCCN(Cc1ccco1)CC1CCCO1)c1ccc(OC(F)(F)F)cc1]

TABLE 79
Characterization data for 79

| Amount (mg) | Yield (%) | Purity (%) | MS (m/z) | Ion type | tᵣ (min) |
|---|---|---|---|---|---|
| 103.6 | 57.7 | 98.89 | 462.9 | [M + H]⁺ | 1.9 |

| | |
|---|---|
| ¹H-NMR (600 | 9.73-10.45 (m, 1 H), 7.90-7.96 (m, 3 H), 7.82 (br s, 1 H), 7.61 (d, J = 8.2 |
| MHz, | Hz, 2 H), 6.75 (dd, J = 13.0, 2.9 Hz, 1 H), 6.58 (br s, 1 H), 4.44 (br dd, |
| DMSO-d₆, | J = 16.7, 3.4 Hz, 2 H), 4.19-4.34 (m, 1 H), 3.70-3.84 (m, 2 H), 3.14 (br |
| 300K) δ | dd, J = 13.1, 4.1 Hz, 1 H), 2.96-3.10 (m, 3 H), 2.81 (quin, J = 6.7 Hz, 2 H), |
| (ppm) | 1.96-2.07 (m, 1 H), 1.78-1.92 (m, 4 H), 1.44-1.56 (m, 1 H) |

Starting material (amine): N¹-(furan-2-ylmethyl)-N¹-((tetrahydrofuran-2-yl)methyl)propane-1,3-diamine.

### N-((1-(4-bromo-2-chlorophenyl)cyclopropyl)methyl)-4-(trifluoromethoxy)benzenesulfonamide (80)

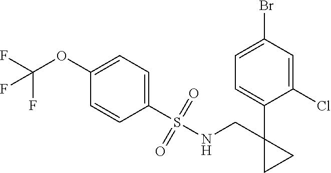 [SMILES: O=S(=O)(NCC1(c2ccc(Br)cc2Cl)CC1)c1ccc(OC(F)(F)F)cc1]

TABLE 80
Characterization data for 80

| Amount (mg) | Yield (%) | Purity (%) | MS (m/z) | Ion type | tᵣ (min) |
|---|---|---|---|---|---|
| 175 | 94.1 | 100 | 485.7 | [M + H]⁺ | 2.88 |

Starting material (amine): (1-(4-bromo-2-chlorophenyl)cyclopropyl)methanamine.

### N-((1-(4-bromobenzyl)cyclopropyl)methyl)-4-(trifluoromethoxy)benzenesulfonamide (81)

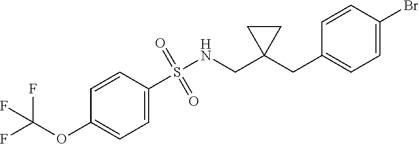 [SMILES: O=S(=O)(NCC1(Cc2ccc(Br)cc2)CC1)c1ccc(OC(F)(F)F)cc1]

TABLE 81
Characterization data for 81

| Amount (mg) | Yield (%) | Purity (%) | MS (m/z) | Ion type | tᵣ (min) |
|---|---|---|---|---|---|
| 163.6 | 88.5 | 96.75 | 463.8 | [M + H]⁺ | 2.85 |

Starting material (amine): (1-(4-bromobenzyl)cyclopropyl)methanamine.

### N-(3-(methylsulfonyl)-5-(trifluoromethyl)benzyl)-4-(trifluoromethoxy)benzenesulfonamide (82)

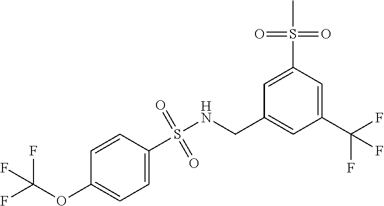 [SMILES: CS(=O)(=O)c1cc(CNS(=O)(=O)c2ccc(OC(F)(F)F)cc2)cc(C(F)(F)F)c1]

TABLE 82
Characterization data for 82

| Amount (mg) | Yield (%) | Purity (%) | MS (m/z) | Ion type | tᵣ (min) |
|---|---|---|---|---|---|
| 122.8 | 62.5 | 93.17 | 477.8 | [M + H]⁺ | 2.44 |

Starting material (amine): (3-(methylsulfonyl)-5-(trifluoromethyl)phenyl)methanamine.

### N-((1-(2-(benzyloxy)ethyl)cyclopropyl)methyl)-4-(trifluoromethoxy)benzenesulfonamide (83)

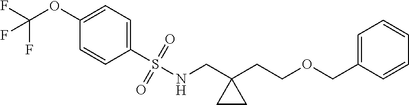 [SMILES: O=S(=O)(NCC1(CCOCc2ccccc2)CC1)c1ccc(OC(F)(F)F)cc1]

TABLE 83
Characterization data for 83

| Amount (mg) | Yield (%) | Purity (%) | MS (m/z) | Ion type | tᵣ (min) |
|---|---|---|---|---|---|
| 159 | 96.5 | 100 | 429.9 | [M + H]⁺ | 2.76 |

Starting material (amine): (1-(2-(benzyloxy)ethyl)cyclopropyl)methanamine.

### N-((1-(2,4-difluorophenyl)-2,2-dimethylcyclopropyl)methyl)-4-(trifluoromethoxy)benzenesulfonamide (84)

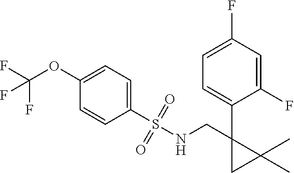 [SMILES: CC1(C)CC1(CNS(=O)(=O)c1ccc(OC(F)(F)F)cc1)c1ccc(F)cc1F]

TABLE 84
Characterization data for 84

| Amount (mg) | Yield (%) | Purity (%) | MS (m/z) | Ion type | tᵣ (min) |
|---|---|---|---|---|---|
| 164.1 | 98.2 | 100 | 435.9 | [M + H]⁺ | 2.82 |

Starting material (amine): (1-(2,4-difluorophenyl)-2,2-dimethylcyclopropyl)methanamine.

### (R)—N-(4,4,4-trifluoro-1-(2,4,5-trifluorophenyl)butan-2-yl)-4-(trifluoromethoxy)benzenesulfonamide (85)

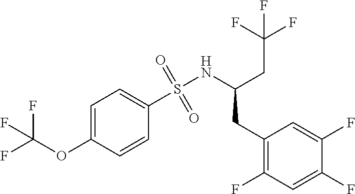 [SMILES: O=S(=O)(N[C@H](Cc1cc(F)c(F)cc1F)CC(F)(F)F)c1ccc(OC(F)(F)F)cc1]

TABLE 85
Characterization data for 85

| Amount (mg) | Yield (%) | Purity (%) | MS (m/z) | Ion type | tᵣ (min) |
|---|---|---|---|---|---|
| 57.7 | 28.8 | 92.31 | 481.8 | [M + H]⁺ | 2.71 |

Starting material (amine): (R)-4,4,4-trifluoro-1-(2,4,5-trifluorophenyl)butan-2-amine.

### N-((4-chlorophenyl)(cyclopentyl)methyl)-4-(trifluoromethoxy)benzenesulfonamide (86)

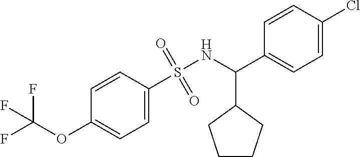 [SMILES: O=S(=O)(NC(c1ccc(Cl)cc1)C1CCCC1)c1ccc(OC(F)(F)F)cc1]

TABLE 86
Characterization data for 86

| Amount (mg) | Yield (%) | Purity (%) | MS (m/z) | Ion type^{a} | tᵣ (min) |
|---|---|---|---|---|---|
| 75.1 | 45.1 | 100 | 432 | [M − H]⁻ | 3 |

^{a}LCMS Method B; Starting material (amine): (4-chlorophenyl)(cyclopentyl)methanamine.

### N-(2-(4-methylpiperidin-1-yl)benzyl)-4-(trifluoromethoxy)benzenesulfonamide (87)

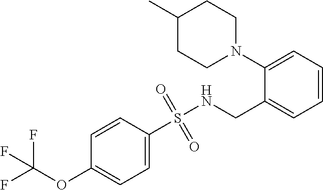 [SMILES: CC1CCN(c2ccccc2CNS(=O)(=O)c2ccc(OC(F)(F)F)cc2)CC1]

TABLE 87
Characterization data for 87

| Amount (mg) | Yield (%) | Purity (%) | MS (m/z) | Ion type | tᵣ (min) |
|---|---|---|---|---|---|
| 150.4 | 90.1 | 98.49 | 428.9 | [M + H]⁺ | 2.47 |

Starting material (amine): (2-(4-methylpiperidin-1-yl)phenyl)methanamine.

### N-((1-(2-chloro-5-(trifluoromethyl)phenyl)cyclopropyl)methyl)-4-(trifluoromethoxy)benzenesulfonamide (88)

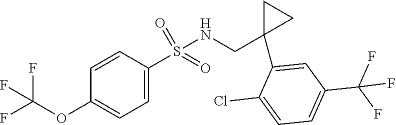 [SMILES: O=S(=O)(NCC1(c2cc(C(F)(F)F)ccc2Cl)CC1)c1ccc(OC(F)(F)F)cc1]

TABLE 88
Characterization data for 88

| Amount (mg) | Yield (%) | Purity (%) | MS (m/z) | Ion type | tᵣ (min) |
|---|---|---|---|---|---|
| 163.5 | 88.5 | 98.44 | 473.8 | [M + H]⁺ | 2.84 |

Starting material (amine): (1-(2-chloro-5-(trifluoromethyl)phenyl)cyclopropyl)methanamine.

### N-(3-(2-(benzyloxy)ethoxy)propyl)-4-(trifluoromethoxy)benzenesulfonamide (89)

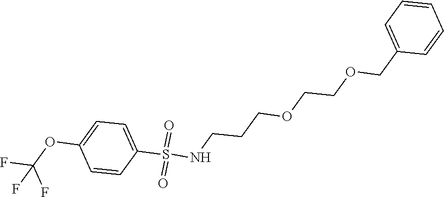 [SMILES: O=S(=O)(NCCCOCCOCc1ccccc1)c1ccc(OC(F)(F)F)cc1]

TABLE 89
Characterization data for 89

| Amount (mg) | Yield (%) | Purity (%) | MS (m/z) | Ion type | tᵣ (min) |
|---|---|---|---|---|---|
| 144.7 | 87 | 100 | 433.9 | [M + H]⁺ | 2.59 |

Starting material (amine): 3-(2-(benzyloxy)ethoxy)propan-1-amine.

### N-(2-((1-ethyl-3-(trifluoromethyl)-1H-pyrazol-5-yl)methoxy)ethyl)-4-(trifluoromethoxy)benzenesulfonamide (90)

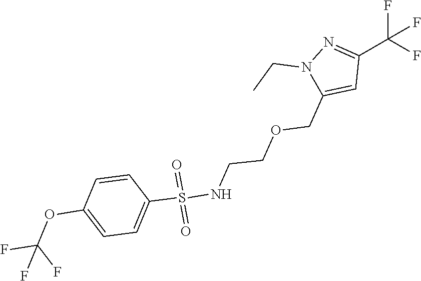 [SMILES: CCn1nc(C(F)(F)F)cc1COCCNS(=O)(=O)c1ccc(OC(F)(F)F)cc1]

TABLE 90
Characterization data for 90

| Amount (mg) | Yield (%) | Purity (%) | MS (m/z) | Ion type | tᵣ (min) |
|---|---|---|---|---|---|
| 161.3 | 91.1 | 100 | 461.9 | [M + H]⁺ | 2.57 |

Starting material (amine): 2-((1-ethyl-3-(trifluoromethyl)-1H-pyrazol-5-yl)methoxy)ethan-1-amine.

### N-((6-benzyl-6-azaspiro[3.4]octan-8-yl)methyl)-4-(trifluoromethoxy)benzenesulfonamide (91)

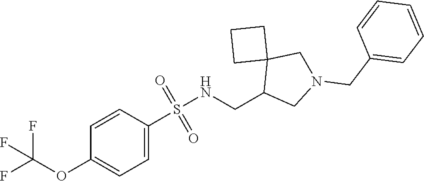 [SMILES: O=S(=O)(NCC1CN(Cc2ccccc2)CC12CCC2)c1ccc(OC(F)(F)F)cc1]

TABLE 91
Characterization data for 91

| Amount (mg) | Yield (%) | Purity (%) | MS (m/z) | Ion type | tᵣ (min) |
|---|---|---|---|---|---|
| 177.7 | 95.7 | 93.95 | 455 | [M + H]⁺ | 1.99 |

Starting material (amine): (6-benzyl-6-azaspiro[3.4]octan-8-yl)methanamine.

### Methyl 2-(2-bromophenyl)-2-((4-(trifluoromethoxy)phenyl)sulfonamido)acetate (92)

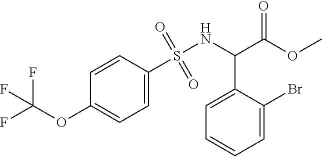 [SMILES: COC(=O)C(NS(=O)(=O)c1ccc(OC(F)(F)F)cc1)c1ccccc1Br]

TABLE 92
Characterization data for 92

| Amount (mg) | Yield (%) | Purity (%) | MS (m/z) | Ion type | tᵣ (min) |
|---|---|---|---|---|---|
| 71 | 36.1 | 91.29 | 469.7 | [M + H]⁺ | 2.55 |

Starting material (amine): methyl 2-amino-2-(2-bromophenyl)acetate.

### N-(1-(1-methylpiperidin-2-yl)propan-2-yl)-4-(trifluoromethoxy)benzenesulfonamide (93)

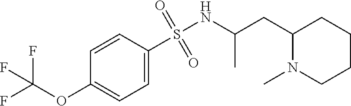 [SMILES: CC(CC1CCCCN1C)NS(=O)(=O)c1ccc(OC(F)(F)F)cc1]

TABLE 93
Characterization data for 93

| Amount (mg) | Yield (%) | Purity (%) | MS (m/z) | Ion type | tᵣ (min) |
|---|---|---|---|---|---|
| 14 | 9.1 | 94.43 | 381 | [M + H]⁺ | 1.76 (1.80) |

| | |
|---|---|
| ¹H-NMR | 9.97-10.30 (m, 1H), 7.93-8.01 (m, 2H), 7.91 (d, J = 8.4 Hz, 1H), 7.61 (d, |
| (DMSO-d₆, | J = 8.1 Hz, 2H), 3.31 (s, 2H), 2.80-2.99 (m, 2H), 2.55-2.76 (m, 3H), |
| 600 MHz) δ | 1.26-2.15 (m, 8H), 0.79-0.95 (m, 3H) |
| (ppm) | |

Starting material (amine): 1-(1-methylpiperidin-2-yl)propan-2-amine.

### Ethyl 3-((4-(trifluoromethoxy)phenyl)sulfonamido)-3-(3-(trifluoromethyl)phenyl)propanoate (94)

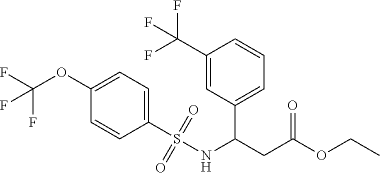 [SMILES: CCOC(=O)CC(NS(=O)(=O)c1ccc(OC(F)(F)F)cc1)c1cccc(C(F)(F)F)c1]

TABLE 94
Characterization data for 94

| Amount (mg) | Yield (%) | Purity (%) | MS (m/z) | Ion type | tᵣ (min) |
|---|---|---|---|---|---|
| 12.3 | 6.4 | 97.6 | 485.8 | [M + H]⁺ | 2.67 |

Starting material (amine): ethyl 3-amino-3-(3-(trifluoromethyl)phenyl)propanoate.

### N-(2-((2-methylcyclohexyl)oxy)ethyl)-4-(trifluoromethoxy)benzenesulfonamide (95)

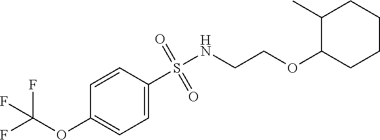 [SMILES: CC1CCCCC1OCCNS(=O)(=O)c1ccc(OC(F)(F)F)cc1]

TABLE 95
Characterization data for 95

| Amount (mg) | Yield (%) | Purity (%) | MS (m/z) | Ion type | tᵣ (min) |
|---|---|---|---|---|---|
| 14.8 | 9.5 | 93.52 | 381 | [M + H]⁺ | 2.76 |

Starting material (amine): 2-((2-methylcyclohexyl)oxy)ethan-1-amine.

### 3-chloro-4-fluoro-N-(2-((4-(trifluoromethoxy)phenyl)sulfonamido)ethyl)benzenesulfonamide (96)

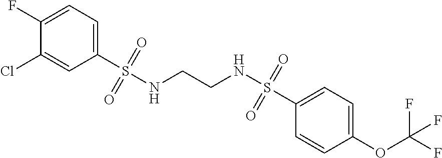 [SMILES: O=S(=O)(NCCNS(=O)(=O)c1ccc(F)c(Cl)c1)c1ccc(OC(F)(F)F)cc1]

TABLE 96
Characterization data for 96

| Amount (mg) | Yield (%) | Purity (%) | MS (m/z) | Ion type | tᵣ (min) |
|---|---|---|---|---|---|
| 17.2 | 9.2 | 97.79 | 476.8 | [M + H]⁺ | 2.52 |

Starting material (amine): N-(2-aminoethyl)-3-chloro-4-fluorobenzenesulfonamide.

### 4-chloro-2-fluoro-N-(2-((4-(trifluoromethoxy)phenyl)sulfonamido)ethyl)benzenesulfonamide (97)

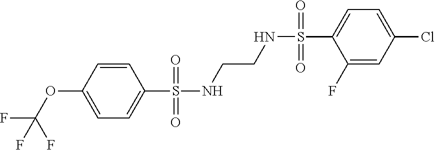 [SMILES: O=S(=O)(NCCNS(=O)(=O)c1ccc(Cl)cc1F)c1ccc(OC(F)(F)F)cc1]

TABLE 97
Characterization data for 97

| Amount (mg) | Yield (%) | Purity (%) | MS (m/z) | Ion type | tᵣ(min) |
|---|---|---|---|---|---|
| 30.4 | 16.4 | 98.5 | 476.8 | [M + H]⁺ | 2.49 |

Starting material (amine): N-(2-aminoethyl)-4-chloro-2-fluorobenzenesulfonamide.

### N-((1-((dimethylamino)methyl)cyclopropyl)methyl)-4-(trifluoromethoxy)benzenesulfonamide (98)

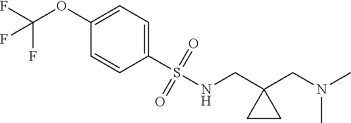 [SMILES: CN(C)CC1(CNS(=O)(=O)c2ccc(OC(F)(F)F)cc2)CC1]

TABLE 98
Characterization data for 98

| Amount | Yield | Purity | MS | Ion | tᵣ |
|---|---|---|---|---|---|
| (mg) | (%) | (%) | (m/z) | type | (min) |
| 32.3 | 23.9 | 100 | 353 | [M + H]⁺ | 1.7 |

Starting material (amine): 1-(1-(aminomethyl)cyclopropyl)-N,N-dimethylmethanamine.

### N-((1-(dimethylamino)-3-methylcyclohexyl)methyl)-4-(trifluoromethoxy)benzenesulfonamide (99)

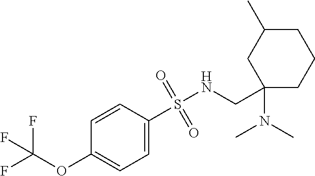 [SMILES: CC1CCCC(CNS(=O)(=O)c2ccc(OC(F)(F)F)cc2)(N(C)C)C1]

TABLE 99
Characterization data for 99

| Amount | Yield | Purity | MS | Ion | tᵣ |
|---|---|---|---|---|---|
| (mg) | (%) | (%) | (m/z) | type | (min) |
| 30.7 | 20.2 | 99.38 | 395 | [M + H]⁺ | 1.85 (1.89) |

Starting material (amine): 1-(aminomethyl)-N,N,3-trimethylcyclohexan-1-amine.

### N-(2-(2,2-dioxidobenzo[c] [1,2,5]thiadiazol-1(3H)-yl)ethyl)-4-(trifluoromethoxy)benzenesulfonamide (100)

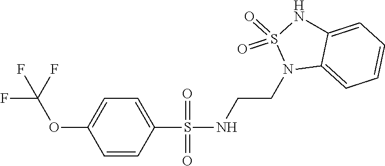 [SMILES: O=S(=O)(NCCN1c2ccccc2NS1(=O)=O)c1ccc(OC(F)(F)F)cc1]

TABLE 100
Characterization data for 100

| Amount | Yield | Purity | MS | Ion | tᵣ |
|---|---|---|---|---|---|
| (mg) | (%) | (%) | (m/z) | type | (min) |
| 39.4 | 22.2 | 94.74 | 437.8 | [M + H]⁺ | 2.34 |

Starting material (amine): 1-(2-aminoethyl)-1,3-dihydrobenzo[c][1,2,5]thiadiazole 2,2-dioxide.

### N-((4-fluorophenyl)(tetrahydrofuran-2-yl)methyl)-4-(trifluoromethoxy)benzenesulfonamide (101)

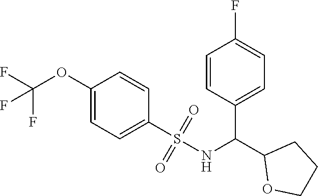 [SMILES: O=S(=O)(NC(c1ccc(F)cc1)C1CCCO1)c1ccc(OC(F)(F)F)cc1]

TABLE 101
Characterization data for 101

| Amount | Yield | Purity | MS | Ion | tᵣ |
|---|---|---|---|---|---|
| (mg) | (%) | (%) | (m/z) | type | (min) |
| 84.2 | 51.7 | 98.72 | 441.9 | [M + Na]⁺ | 2.57 |

| | |
|---|---|
| ¹H-NMR (600 | 8.24-8.57 (m, 1 H), 7.58-7.66 (m, 2 H), 7.24-7.32 (m, |
| MHz, DMSO- | 2 H), 7.00-7.16 (m, 3 H), 6.77-6.90 (m, 1 H), 4.07- |
| d₆, 300K) δ | 4.38 (m, 1 H), 3.83-4.00 (m, 1 H), 3.46 (s, 2 H), |
| (ppm) | 1.45-1.94 (m, 4 H) |

Starting material (amine): (4-fluorophenyl)(tetrahydrofuran-2-yl)methanamine.

### N-((3-methylcyclohexyl)(o-tolyl)methyl)-4-(trifluoromethoxy)benzenesulfonamide (102)

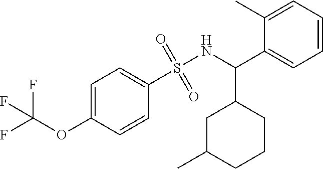 [SMILES: Cc1ccccc1C(NS(=O)(=O)c1ccc(OC(F)(F)F)cc1)C1CCCC(C)C1]

TABLE 102
Characterization data for 102

| Amount | Yield | Purity | MS | Ion | tᵣ |
|---|---|---|---|---|---|
| (mg) | (%) | (%) | (m/z) | type | (min) |
| 106.3 | 60.1 | 95.83 | 463.9 | [M + Na]⁺ | 3.09 |

Starting material (amine): (3-methylcyclohexyl)(o-tolyl)methanamine.

### N-((2-methylcyclopropyl)(phenyl)methyl)-4-(trifluoromethoxy)benzenesulfonamide (103)

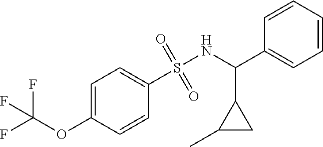 [SMILES: CC1CC1C(NS(=O)(=O)c1ccc(OC(F)(F)F)cc1)c1ccccc1]

TABLE 103
Characterization data for 103

| Amount | Yield | Purity | MS | Ion | tᵣ |
|---|---|---|---|---|---|
| (mg) | (%) | (%) | (m/z) | type^{a} | (min) |
| 130 | 81.4 | 92.61 | 384 | [M − H]⁻ | 2.70 (2.72) |

| | |
|---|---|
| ¹H-NMR | 8.44-8.59 (m, 1H), 7.66-7.75 (m, 2H), 7.33-7.43 (m, |
| (DMSO-d₆, | 2H), 6.97-7.22 (m, 5H), 3.54-3.72 (m, 1H), 0.81 (d, |
| 600 MHz) δ | J = 6.0 Hz, 3H), 0.73-0.80 (m, 1H), 0.62-0.69 |
| (ppm) | (m, 1H), 0.05-0.43 (m, 2H) |

^{a}LCMS Method B; Starting material (amine): (2-methylcyclopropyl)(phenyl)methanamine.

### N-(2-(methylsulfonyl)-1-(p-tolyl)propyl)-4-(trifluoromethoxy)benzenesulfonamide (104)

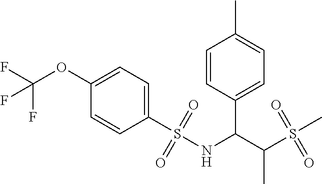 [SMILES: Cc1ccc(C(NS(=O)(=O)c2ccc(OC(F)(F)F)cc2)C(C)S(C)(=O)=O)cc1]

TABLE 104
Characterization data for 104

| Amount | Yield | Purity | MS | Ion | tᵣ |
|---|---|---|---|---|---|
| (mg) | (%) | (%) | (m/z) | type | (min) |
| 152 | 86.7 | 98.79 | 451.9 | [M + H]⁺ | 2.42 (2.45) |

| | |
|---|---|
| ¹H-NMR | 8.45-8.75 (m, 1H), 7.44-7.68 (m, 2H), 7.12-7.31 (m, |
| (DMSO-d₆, | 2H), 6.70-6.99 (m, 4H), 4.55-5.08 (m, 1H), 3.47 (quin, |
| 600 MHz) δ | J = 7.4 Hz, 1H), 2.66-3.10 (m, 3H), 2.13 (d, J = |
| (ppm) | 3.1 Hz, 3H), 0.84-1.38 (m, 3H) |

Starting material (amine): 2-(methylsulfonyl)-1-(p-tolyl)propan-1-amine.

### Tert-butyl (1-phenyl-2-((4-(trifluoromethoxy)phenyl)sulfonamido)propyl)carbamate (105)

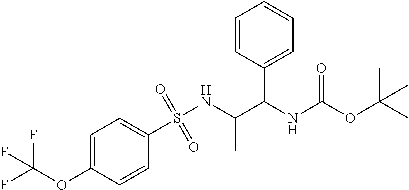 [SMILES: CC(NS(=O)(=O)c1ccc(OC(F)(F)F)cc1)C(NC(=O)OC(C)(C)C)c1ccccc1]

TABLE 105
Characterization data for 105

| Amount | Yield | Purity | MS | Ion | tᵣ |
|---|---|---|---|---|---|
| (mg) | (%) | (%) | (m/z) | type^{a} | (min) |
| 167 | 88.2 | 96.15 | 473 | [M − H]⁻ | 2.84 (2.89) |
| ¹H-NMR | 7.80 (br d, J = 8.8 Hz, 1H), 7.21-7.26 (m, | | | | |
| (DMSO-d₆, | 1H), 6.81-8.04 (m, 9H), 4.31-4.68 (m, 1H), 3.37- | | | | |
| 600 MHz) δ | 3.66 (m, 1H), 1.16-1.53 (m, 9H), 0.83-0.97 (m, 3H) | | | | |
| (ppm) | | | | | |

^{a}LCMS Method B; Starting material (amine): tert-butyl (2-amino-1-phenylpropyl)carbamate.

### Rac-N-(((2S,3R)-2-(4-methoxyphenyl)-1-methylpyrrolidin-3-yl)methyl)-4-(trifluoromethoxy)benzenesulfonamide (106)

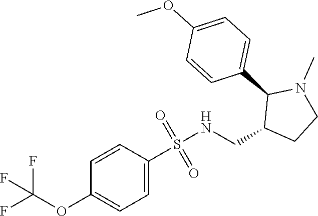 [SMILES: COc1ccc([C@@H]2[C@@H](CNS(=O)(=O)c3ccc(OC(F)(F)F)cc3)CCN2C)cc1]

TABLE 106
Characterization data for 106

| Amount | Yield | Purity | MS | Ion | tᵣ |
|---|---|---|---|---|---|
| (mg) | (%) | (%) | (m/z) | type | (min) |
| 143.2 | 81.8 | 97.47 | 444.9 | [M + H]⁺ | 1.9 |

Starting material (amine): rac-((2S,3R)-2-(4-methoxyphenyl)-1-methylpyrrolidin-3-yl)methanamine.

### Methyl 2-phenyl-2-((4-(trifluoromethoxy)phenyl)sulfonamido)butanoate (107)

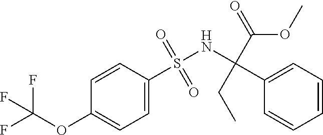 [SMILES: CCC(NS(=O)(=O)c1ccc(OC(F)(F)F)cc1)(C(=O)OC)c1ccccc1]

TABLE 107
Characterization data for 107

| Amount | Yield | Purity | MS | Ion | tᵣ |
|---|---|---|---|---|---|
| (mg) | (%) | (%) | (m/z) | type | (min) |
| 5.29 | 3 | 91.92 | 439.9 | [M + Na]⁺ | 2.63 |

Starting material (amine): methyl 2-amino-2-phenylbutanoate.

### 4-nitro-N-(2-((4-(trifluoromethoxy)phenyl)sulfonamido)ethyl)benzenesulfonamide (108)

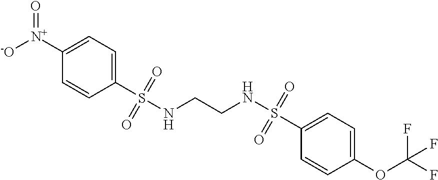

TABLE 108
Characterization data for 108

| Amount | Yield | Purity | MS | Ion | tᵣ |
|---|---|---|---|---|---|
| (mg) | (%) | (%) | (m/z) | type | (min) |
| 3.3 | 1.7 | 90.99 | 469.8 | [M + H]⁺ | 2.43 |

Starting material (amine): N-(2-aminoethyl)-4-nitrobenzenesulfonamide.

### 4-methyl-N-(thiazol-2-yl)-2-((4-(trifluoromethoxy)phenyl)sulfonamido)pentanamide (109)

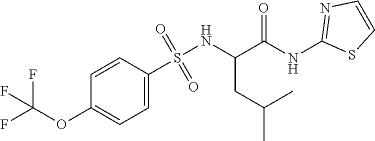 [SMILES: CC(C)CC(NS(=O)(=O)c1ccc(OC(F)(F)F)cc1)C(=O)Nc1nccs1]

TABLE 109
Characterization data for 109

| Amount | Yield | Purity | MS | Ion | tᵣ |
|---|---|---|---|---|---|
| (mg) | (%) | (%) | (m/z) | type | (min) |
| 6 | 3.4 | 93.86 | 437.9 | [M + H]⁺ | 2.41 |

Starting material (amine): 2-amino-4-methyl-N-(thiazol-2-yl)pentanamide.

### Ethyl 3-(tetrahydrofuran-3-yl)-2-((4-(trifluoromethoxy)phenyl)sulfonamido)propanoate (110)

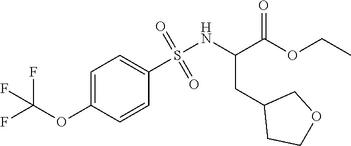 [SMILES: CCOC(=O)C(CC1CCOC1)NS(=O)(=O)c1ccc(OC(F)(F)F)cc1]

TABLE 110
Characterization data for 110

| Amount | Yield | Purity | MS | Ion | tᵣ |
|---|---|---|---|---|---|
| (mg) | (%) | (%) | (m/z) | type | (min) |
| 5 | 3.1 | 98.77 | 411.9 | [M + H]⁺ | 2.31 |

Starting material (amine): ethyl 2-amino-3-(tetrahydrofuran-3-yl)propanoate.

### Methyl 3-(2-hydroxyphenyl)-2-((4-(trifluoromethoxy)phenyl)sulfonamido)propanoate (111)

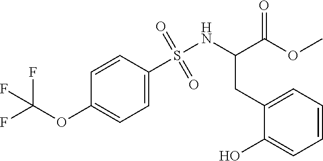 [SMILES: COC(=O)C(Cc1ccccc1O)NS(=O)(=O)c1ccc(OC(F)(F)F)cc1]

TABLE 111
Characterization data for 111

| Amount | Yield | Purity | MS | Ion | tᵣ |
|---|---|---|---|---|---|
| (mg) | (%) | (%) | (m/z) | type | (min) |
| 94 | 52.3 | 89.48 | 419.9 | [M + H]⁺ | 2.84 |

Starting material (amine): methyl 2-amino-3-(2-hydroxyphenyl)propanoate.

### Methyl 3-(3-fluoro-4-hydroxyphenyl)-2-((4-(trifluoromethoxy)phenyl)sulfonamido)propanoate (112)

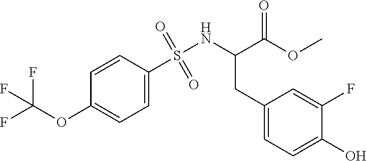 [SMILES: COC(=O)C(Cc1ccc(O)c(F)c1)NS(=O)(=O)c1ccc(OC(F)(F)F)cc1]

TABLE 112
Characterization data for 112

| Amount | Yield | Purity | MS | Ion | tᵣ |
|---|---|---|---|---|---|
| (mg) | (%) | (%) | (m/z) | type | (min) |
| 117 | 61.7 | 88.47 | 437.8 | [M + H]⁺ | 1.93 (1.96) |

Starting material (amine): methyl 2-amino-3-(3-fluoro-4-hydroxyphenyl)propanoate.

### Example 3: 3-(4-isopropylphenyl)-3-((4-(trifluoromethoxy)phenyl)sulfonamido)propanoic acid (113)

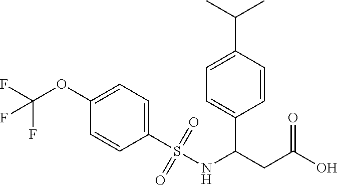 [SMILES: CC(C)c1ccc(C(CC(=O)O)NS(=O)(=O)c2ccc(OC(F)(F)F)cc2)cc1]

### Step 1: Synthesis of methyl 3-(4-isopropylphenyl)-3-[[4-(trifluoromethoxy)phenyl]sulfonylamino]propanoate

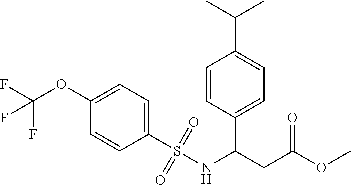 [SMILES: COC(=O)CC(NS(=O)(=O)c1ccc(OC(F)(F)F)cc1)c1ccc(C(C)C)cc1]

The compound was prepared following the general procedure for sulfonamide synthesis as described in Example 2: The reaction vial was charged with methyl 3-amino-3-(4-isopropylphenyl)propanoate (0.4221 mmol, 1.1 eq) and Et₃N (1.5348 mmol, 4 eq, 214 μL). A solution of 4-(trifluoromethoxy)benzenesulfonyl chloride in DCM (0.384 mmol, 1.0 eq, 2 mL, 0.192 mmol/mL) was distributed into the reaction vial. The reaction mixture was stirred at 25° C. overnight. The reaction mixture was filtered on a 3 mL ChemElut cartridge (pre-treated with HCl 1M (3 mL)) and eluted with 2 ml of DCM. The obtained fraction was concentrated under nitrogen flow at rt for 2 h, then dried under vacuum at 40° C. overnight to obtain the title compound (160.8 mg, 86.5% yield, tᵣ=2.71 min). LCMS (Method A): m/z found 445.9 [M+H]⁺.

### Step 2: Synthesis of 3-(4-isopropylphenyl)-3-((4-(trifluoromethoxy)phenyl)sulfonamido)propanoic acid (113)

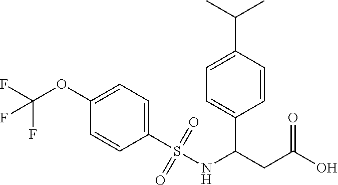 [SMILES: CC(C)c1ccc(C(CC(=O)O)NS(=O)(=O)c2ccc(OC(F)(F)F)cc2)cc1]

In a sealed flask, to a stirred solution of methyl 3-(4-isopropylphenyl)-3-[[4-(trifluoromethoxy)phenyl]sulfonylamino]propanoate (133 mg, 0.299 mmol) in MeOH (1 mL) at RT was added 1 M NaOH (299 μL, 0.299 mmol). The reaction mixture was stirred at 80° C. for 3 h. The reaction mixture was stirred at RT for 2 h. The reaction mixture was stirred at RT overnight. This reaction mixture was concentrated under N₂ flow for 3 h and dried under vacuum at 40° C. overnight. The crude was purified by flash chromatography on silica gel using a gradient of MeOH in DCM. The desired fractions were combined and concentrated to afford the expected compound (65.5 mg, 44.01% yield, 90.96% purity, tᵣ=2.61 min). LCMS (Method C): m/z found 430 [M−H]⁻.

### Example 4: tert-butyl (R)-(2-phenyl-2-((4-(trifluoromethoxy)phenyl)sulfonamido)ethyl)carbamate (114)

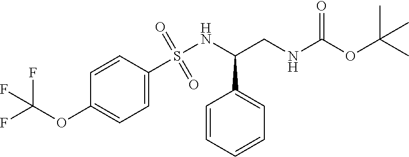 [SMILES: CC(C)(C)OC(=O)NC[C@H](NS(=O)(=O)c1ccc(OC(F)(F)F)cc1)c1ccccc1]

In a round bottomed flask under nitrogen, 4-(trifluoromethoxy)benzenesulfonyl chloride (0.16 mL, 0.959 mmol) was added to a stirred solution of tert-butyl [(2R)-2-amino-2-phenylethyl]carbamate (249 mg, 1.06 mmol) and triethylamine (535 μL, 3.84 mmol) in anhydrous DCM (9.5925 mL). The solution was stirred at rt for 20 h. The crude was concentrated in vacuo, dried under vacuum and purified by flash chromatography on silica gel using a gradient of EtOAc in Heptane from 10% to 50% to afford the title compound as a white powder (327 mg, 74% yield, 99.2% purity, tᵣ=2.71 min). LCMS (Method C): m/z found 460.9 [M+H]⁺; ¹H-NMR (DMSO-d₆, 400 MHz): δ (ppm) 8.37 (s, 1H), 7.64 (d, J=8.8 Hz, 2H), 7.30 (br d, J=8.3 Hz, 2H), 7.03-7.12 (m, 5H), 6.76 (t, J=6.0 Hz, 1H), 4.38 (br t, J=7.3 Hz, 1H), 3.03-3.16 (m, 2H), 1.30 (s, 9H).

### Example 5: tert-butyl (S)-(2-phenyl-2-((4-(trifluoromethoxy)phenyl)sulfonamido)ethyl)carbamate (115)

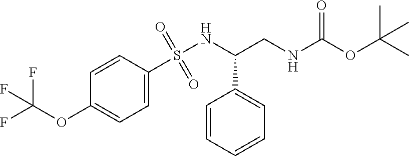 [SMILES: CC(C)(C)OC(=O)NC[C@@H](NS(=O)(=O)c1ccc(OC(F)(F)F)cc1)c1ccccc1]

In a sealed tube, a solution of 4-(trifluoromethoxy)benzenesulfonyl chloride (250 mg, 0.959 mmol) in DCM (1.2 mL) was added to a stirred solution of tert-butyl [(2S)-2-amino-2-phenylethyl]carbamate (249 mg, 1.06 mmol) and triethylamine (535 μL, 3.84 mmol) in DCM (6 mL). The solution was stirred at rt for 20 h and was diluted with water. The aqueous layer was extracted with DCM, the combined organic layers were washed with a saturated aqueous solution of NH₄Cl, a saturated aqueous solution of NaHCO₃, dried over sodium sulfate, filtered and concentrated. The crude was purified by flash chromatography on silica gel using a gradient of (MeOH+2% NH₄OH) in DCM from 0.2% to 1% to afford the title compound as a white foam (270 mg, 60% yield, 99.5% purity, tᵣ=2.71 min). LCMS (Method C): m/z found 461 [M+H]⁺; ¹H-NMR (DMSO-d₆, 400 MHz): δ (ppm) 8.37 (d, J=9.1 Hz, 1H), 7.64 (d, J=8.6 Hz, 2H), 7.30 (br d, J=8.3 Hz, 2H), 7.00-7.16 (m, 5H), 6.77 (t, J=5.8 Hz, 1H), 4.25-4.45 (m, 1H), 2.98-3.19 (m, 2H), 1.30 (s, 9H).

### Example 6: (R)—N-(2-amino-1-phenylethyl)-4-(trifluoromethoxy)benzenesulfonamide (116)

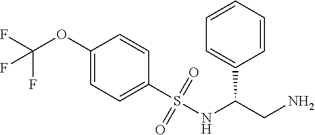 [SMILES: NC[C@H](NS(=O)(=O)c1ccc(OC(F)(F)F)cc1)c1ccccc1]

In a round-bottomed flask, to a stirred solution of tert-butyl N-[(2R)-2-phenyl-2-[[4-(trifluoromethoxy)phenyl]sulfonylamino]ethyl]carbamate (220 mg, 0.477 mmol) in anhydrous 1,4-dioxane (1.9085 mL) at rt was added 4 M hydrogen chloride (1.9 mL, 7.63 mmol). The reaction mixture was stirred at rt overnight. The reaction mixture was basified with a saturated NaHCO₃ solution and extracted three times with DCM. The organic layers were washed with brine, dried under vacuum to afford the title compound as a white powder (150 mg, 87.2% yield, 99.4% purity, tᵣ=1.2 min). LCMS (Method C): m/z found 360.9 [M+H]⁺; ¹H-NMR (500 MHz, DMSO-d₆) δ (ppm) 7.66 (d, J=7.96 Hz, 2H), 7.33 (d, J=8.07 Hz, 2H), 7.05-7.12 (m, 5H), 4.17 (dd, J=5.87, 7.58 Hz, 1H), 3.43-4.01 (m, 3H), 2.59-2.69 (m, 2H).

### Example 7: (S)—N-(2-amino-1-phenylethyl)-4-(trifluoromethoxy)benzenesulfonamide (117)

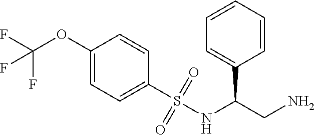 [SMILES: NC[C@@H](NS(=O)(=O)c1ccc(OC(F)(F)F)cc1)c1ccccc1]

In a sealed tube, 4 M hydrogen chloride in dioxane (923 μL, 3.69 mmol) was added to a stirred solution of tert-butyl N-[(2S)-2-phenyl-2-[[4-(trifluoromethoxy)phenyl]sulfonylamino]ethyl]carbamate (100%, 170 mg, 0.369 mmol) in 1,4-dioxane (1 mL). The mixture was stirred at rt for 20 h. The suspension was concentrated and the residue was dissolved in DCM and a saturated aqueous solution of NaHCO₃. The organic layer was washed with a saturated aqueous solution of NaHCO₃, dried over sodium sulfate, filtered and concentrated to afford the title compound as a white powder (115 mg, 86% yield, 99.5% purity, tᵣ=1.21 min). LCMS (Method C): m/z found 360.9 [M+H]⁺; 1H-NMR (500 MHz, DMSO-d₆) δ (ppm) 7.66 (d, J=7.87 Hz, 2H), 7.32 (d, J=8.07 Hz, 2H), 7.05-7.12 (m, 5H), 4.17 (dd, J=5.87, 7.34 Hz, 1H), 3.71 (br s, 3H), 2.59-2.69 (m, 2H).

### Example 8: N-(1-phenyl-2-(4-propoxypiperidin-1-yl)ethyl)-4-(trifluoromethoxy)benzenesulfonamide (118)

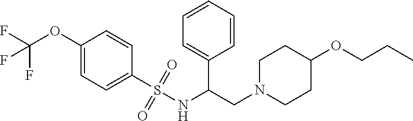 [SMILES: CCCOC1CCN(CC(NS(=O)(=O)c2ccc(OC(F)(F)F)cc2)c2ccccc2)CC1]

In a sealed tube, 4-(trifluoromethoxy)benzenesulfonyl chloride (47 μL, 0.267 mmol) was added to a stirred solution of 1-phenyl-2-(4-propoxy-1-piperidyl)ethanamine (70 mg, 0.267 mmol) and triethylamine (149 μL, 1.07 mmol) in anhydrous DCM (2.8 mL). The solution was stirred at room temperature for 20 h. Water (10 mL) was added and the aqueous layer was extracted with dichloromethane (2×8 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 4%. The residue was triturated in 2 M hydrogen chloride in diethyl ether (1.4 mL, 2.80 mmol), filtered, washed with diethyl ether and dried under vacuum at 60° C. for 20 h to afford the title compound as a white powder (89.1 mg, 64% yield, 100% purity, tᵣ=1.67 min). LCMS (Method C): m/z found 487.1 [M+H]⁺; ¹H-NMR (DMSO-d₆, 500 MHz): δ (ppm) 9.87-9.98 (m, 1H), 8.77 (d, J=9.8 Hz, 1H), 7.58-7.65 (m, 2H), 7.25 (br d, J=8.6 Hz, 2H), 7.14-7.17 (m, 2H), 7.03-7.12 (m, 3H), 4.79-4.92 (m, 1H), 3.53-3.69 (m, 1H), 3.36-3.51 (m, 4H), 3.02-3.28 (m, 4H), 1.58-2.16 (m, 4H), 1.52 (dq, J=11.2, 6.9 Hz, 2H), 0.89 (dt, J=10.5, 7.3 Hz, 3H).

### Example 9: N-(2-(dimethylamino)-1-(m-tolyl)ethyl)-4-(trifluoromethoxy)benzenesulfonamide (119)

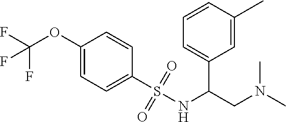 [SMILES: Cc1cccc(C(CN(C)C)NS(=O)(=O)c2ccc(OC(F)(F)F)cc2)c1]

In a sealed tube, a solution of 4-(trifluoromethoxy)benzenesulfonyl chloride (50 mg, 0.192 mmol) in DCM (0.24 mL) was added to a stirred solution of N′,N′-dimethyl-1-(m-tolyl)ethane-1,2-diamine (38 mg, 0.211 mmol) and triethylamine (107 uL, 0.767 mmol) in DCM (1.2 mL). The solution was stirred at rt for 20 h and was diluted with DCM and an half saturated aqueous solution of NaHCO₃. The aqueous layer was extracted with DCM, the combined organic layers were dried over sodium sulfate, filtered and concentrated. The crude was purified by flash chromatography on silica gel using a gradient of (MeOH+2% NH₄OH) in DCM from 0.2% to 5%. The desired fractions were concentrated, the residue was triturated with pentane and the suspension was filtered. The residue was washed with pentane and dried under reduced pressure at 40° C. for 16 h to afford the title compound as a white powder. (31 mg, 39% yield, 99% purity, tᵣ=1.39 min). LCMS (Method C): m/z found 403 [M+H]⁺; 1H-NMR (DMSO-d₆, 500 MHz): δ (ppm) 8.18 (br d, J=4.6 Hz, 1H), 7.68 (d, J=8.8 Hz, 2H), 7.34 (d, J=8.1 Hz, 2H), 6.95-7.06 (m, 1H), 6.83-6.94 (m, 3H), 4.26 (br s, 1H), 2.45 (dd, J=12.5, 9.0 Hz, 1H), 2.23 (br dd, J=12.5, 5.9 Hz, 1H), 2.14 (s, 3H), 2.03 (s, 6H).

### Example 10: N-(2-(dimethylamino)-1-(3-(trifluoromethyl)phenyl)ethyl)-4-(trifluoromethoxy)benzenesulfonamide (120)

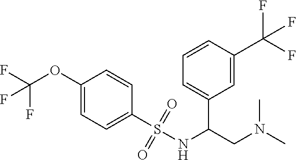 [SMILES: CN(C)CC(NS(=O)(=O)c1ccc(OC(F)(F)F)cc1)c1cccc(C(F)(F)F)c1]

In a sealed tube, a solution of N′,N′-dimethyl-1-[3-(trifluoromethyl)phenyl]ethane-1,2-diamine (48 mg, 0.207 mmol) in DCM (0.25 mL) was added to a stirred solution of triethylamine (105 μL, 0.752 mmol) and 4-(trifluoromethoxy)benzenesulfonyl chloride (98%, 50 mg, 0.188 mmol) in DCM (1.2 mL). The solution was stirred at rt for 2.5 h and was diluted with water. The aqueous layer was extracted twice with DCM, the combined organic layers were washed with a saturated aqueous solution of NH₄Cl, a saturated aqueous solution of NaHCO₃ and a saturated aqueous solution of NaCl, dried using a phase separator and concentrated. The crude was purified by flash chromatography on silica gel using a gradient of MeOH in DCM from 0.2% to 5%. The desired fraction were concentrated, the residue was solubilized in 1.5 mL of Et₂O and of HCl (150 μL, 2 N in Et₂O) was added. The mixture was stirred for 2 h, filtered under N₂, washed with Et₂O and dried under vacuum at 40° C. for 18 h to afford the title compound as a white powder (67.7 mg, 73% yield, 99.2% purity, tᵣ=1.5 min). LCMS (Method C): m/z found 457 [M+H]⁺; ¹H-NMR (500 MHz, DMSO-d₆) δ (ppm) 9.44-10.03 (m, 1H) 8.87-9.25 (m, 1H) 7.59-7.63 (m, 2H) 7.52-7.58 (m, 2H) 7.40-7.47 (m, 1H) 7.30-7.37 (m, 1H) 7.17-7.22 (m, 2H) 4.66-5.31 (m, 1H) 3.37-3.52 (m, 1H) 3.18-3.27 (m, 1H) 2.74-3.03 (m, 6H).

### Example 11: (R)—N-(1-(3,4-dichlorophenyl)-2-hydroxyethyl)-4-(trifluoromethoxy)benzenesulfonamide (121)

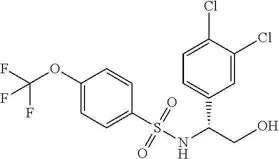 [SMILES: O=S(=O)(N[C@@H](CO)c1ccc(Cl)c(Cl)c1)c1ccc(OC(F)(F)F)cc1]

In a sealed tube, a solution of 4-(trifluoromethoxy)benzenesulfonyl chloride (98%, 50 mg, 0.188 mmol) in DCM (0.25 mL) was added to a stirred solution of (2R)-2-amino-2-(3,4-dichlorophenyl)ethanol hydrochloride (50 mg, 0.207 mmol) and triethylamine (131 μL, 0.940 mmol) in DCM (1.2 mL). The solution was stirred at rt for 2.5 h and was concentrated. The crude was purified by flash chromatography on silica gel using a gradient of MeOH in DCM from 0.2% to 5%. The desired fractions were concentrated. The residue was dissolved in DCM and pentane was added. The resulting precipitate was filtered, washed with pentane and dried under vacuum at 40° C. for 2 days to afford the title compound as a white powder (54.1 mg, 67% yield, 100% purity, tᵣ=2.44 min). LCMS (Method C): m/z found 427.8 [M−H]⁻; ¹H-NMR (600 MHz, DMSO-d₆) δ (ppm) 8.25-8.56 (m, 1H) 7.65-7.73 (m, 2H) 7.33-7.40 (m, 3H) 7.27-7.32 (m, 1H) 7.06-7.14 (m, 1H) 4.79-5.09 (m, 1H) 4.06-4.46 (m, 1H) 3.38-3.56 (m, 2H).

### Example 12: (S)—N-(1-(3,4-dichlorophenyl)-2-hydroxyethyl)-4-(trifluoromethoxy)benzenesulfonamide (122)

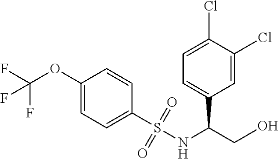 [SMILES: O=S(=O)(N[C@H](CO)c1ccc(Cl)c(Cl)c1)c1ccc(OC(F)(F)F)cc1]

In a sealed tube, a solution of 4-(trifluoromethoxy)benzenesulfonyl chloride (98%, 50 mg, 0.188 mmol) in DCM (0.25 mL) was added to a stirred solution of (2S)-2-amino-2-(3,4-dichlorophenyl)ethanol hydrochloride (50 mg, 0.207 mmol) and triethylamine (131 μL, 0.940 mmol) in DCM (1.2 mL). The solution was stirred at rt for 2.5 h and was concentrated. The crude was purified by flash chromatography on silica gel using a gradient of MeOH in DCM from 0.2% to 5%. The desired fractions were concentrated. The residue was triturated in pentane, filtered, washed with pentane, and dried under vacuum at 40° C. for 2 days to afford the title compound as a white powder (54.5 mg, 67% yield, 100% purity, tᵣ=2.44 min). LCMS (Method C): m/z found 100 [M−H]⁻; ¹H-NMR (600 MHz, DMSO-d₆) δ (ppm) 8.25-8.54 (m, 1H) 7.67-7.72 (m, 2H) 7.33-7.39 (m, 3H) 7.30 (d, J=2.05 Hz, 1H) 7.10 (dd, J=8.29, 1.98 Hz, 1H) 4.77-5.12 (m, 1H) 4.31 (t, J=6.31 Hz, 1H) 3.38-3.60 (m, 2H).

### Example 13: N-(2-(dimethylamino)-1-(p-tolyl)ethyl)-4-(trifluoromethoxy)benzenesulfonamide (123)

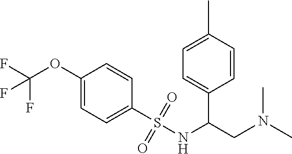 [SMILES: Cc1ccc(C(CN(C)C)NS(=O)(=O)c2ccc(OC(F)(F)F)cc2)cc1]

In a sealed tube, a solution of 4-(trifluoromethoxy)benzenesulfonyl chloride (0.032 mL, 0.192 mmol) in DCM (0.24 mL) was added to a stirred solution of N′,N′-dimethyl-1-(p-tolyl)ethane-1,2-diamine (38 mg, 0.211 mmol) and triethylamine (107 μL, 0.767 mmol) in DCM (1.2 mL). The solution was stirred at rt for 20 h and was diluted with water. The aqueous layer was extracted with DCM, the combined organic layers were washed with a saturated aqueous solution of NH₄Cl, a saturated aqueous solution of NaHCO₃, dried over sodium sulfate, filtered and concentrated. The crude was purified by flash chromatography on silica gel using a gradient of (MeOH+2% NH₄OH) in DCM from 0.2% to 5%. The desired fractions were concentrated and the residue was dissolved in pentane and concentrated. The residue was dissolved in a minimum amount of pentane and left to stand for 15 min. The obtained suspension was concentrated to afford the title compound as a white powder. (41 mg, 53% yield, 100% purity, tᵣ=1.41 min). LCMS (Method C): m/z found 402.9 [M+H]⁺; ¹H-NMR (500 MHz, DMSO-d₆) δ (ppm) 8.17 (br d, J=6.11 Hz, 1H), 7.67 (d, J=7.73 Hz, 2H), 7.34 (d, J=8.07 Hz, 2H), 6.97 (d, J=8.07 Hz, 2H), 6.90 (d, J=7.82 Hz, 2H), 4.26 (br d, J=5.87 Hz, 1H), 2.35-2.48 (m, 1H), 2.11-2.26 (m, 4H), 2.02 (s, 6H).

### Example 14: 1-methyl-N-(2-phenyl-2-((4-(trifluoromethoxy)phenyl)sulfonamido)ethyl)-1H-pyrrole-3-sulfonamide (124)

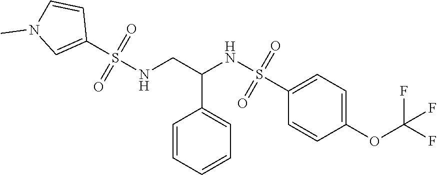 [SMILES: Cn1ccc(S(=O)(=O)NCC(NS(=O)(=O)c2ccc(OC(F)(F)F)cc2)c2ccccc2)c1]

In a sealed tube, a solution of 4-(trifluoromethoxy)benzenesulfonyl chloride (32 μL, 0.192 mmol) in DCM (0.24 mL) was added to a stirred solution of N-(2-amino-2-phenyl-ethyl)-1-methyl-pyrrole-3-sulfonamide (59 mg, 0.211 mmol) and triethylamine (107 μL, 0.767 mmol) in DCM (1.2 mL). The solution was stirred at rt for 20 h and was diluted with water. The aqueous layer was extracted with DCM, the combined organic layers were washed with a saturated aqueous solution of NH₄Cl, a saturated aqueous solution of NaHCO₃, dried over sodium sulfate, filtered and concentrated. The crude was purified by flash chromatography on silica gel using a gradient of (MeOH+2% NH₄OH) in DCM from 0.2% to 5%. The desired fractions were concentrated and the residue was sonicated in Et₂O. The obtained suspension was filtered, the residue was washed with a minimum of Et₂O and was dried under reduced pressure at 40° C. for 4 h to afford the title compound as a yellow powder. (38 mg, 38% yield, 97.6% purity, tᵣ=2.35 min). LCMS (Method C): m/z found 504 [M+H]⁺; ¹H-NMR (500 MHz, DMSO-d₆) δ (ppm) 8.39 (d, J=9.05 Hz, 1H) 7.57-7.67 (m, 2H) 7.27-7.33 (m, 2H) 7.13-7.16 (m, 1H) 7.05-7.12 (m, 4H) 6.97-7.04 (m, 2H) 6.77-6.82 (m, 1H) 6.13-6.17 (m, 1H) 4.24-4.46 (m, 1H) 3.63 (s, 3H) 2.73-2.98 (m, 2H).

### Example 15: N-(1-(4-chlorophenyl)-2-(dimethylamino)ethyl)-4-(trifluoromethoxy)benzenesulfonamide (125)

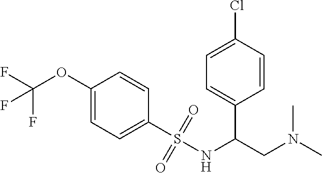 [SMILES: CN(C)CC(NS(=O)(=O)c1ccc(OC(F)(F)F)cc1)c1ccc(Cl)cc1]

In a sealed tube, a solution of 4-(trifluoromethoxy)benzenesulfonyl chloride (98%, 50 mg, 0.188 mmol) in DCM (0.25 mL) was added to a stirred solution of 1-(4-chlorophenyl)-N′,N′-dimethyl-ethane-1,2-diamine (41 mg, 0.207 mmol) and triethylamine (105 μL, 0.752 mmol) in DCM (1.2 mL). The solution was stirred at rt for 2.5 h and was diluted with water. The aqueous layer was extracted twice with DCM, the combined organic layers were washed with a saturated aqueous solution of NH₄Cl, a saturated aqueous solution of NaHCO₃, a saturated aqueous solution of NaCl, and dried using a phase separator and concentrated. The crude product was purified by flash chromatography on silica gel using a gradient of MeOH in DCM from 0.2% to 5%. The desired fractions were concentrated. The residue was triturated in pentane, filtered, washed with pentane, and dried under vacuum at 40° C. for 2 days to afford the title compound as a white powder (55.5 mg, 68% yield, 97.1% purity, tᵣ=1.42 min). LCMS (Method C): m/z 422.9 [M+H]⁺; ¹H-NMR (DMSO-d₆, 600 MHz): δ (ppm) 8.29 (br s, 1H), 7.70 (d, J=8.0 Hz, 2H), 7.37 (d, J=8.1 Hz, 2H), 7.12-7.17 (m, 4H), 4.31 (br s, 1H), 2.20-2.45 (m, 2H), 2.02 (s, 6H).

### Example 16: N-(1-(3,4-dichlorophenyl)-2-(dimethylamino)ethyl)-N′-methyl-4-(trifluoromethoxy)benzenesulfonimidamide (126) and N′-(1-(3,4-dichlorophenyl)-2-(dimethylamino)ethyl)-N-methyl-4-(trifluoromethoxy)benzenesulfonimidamide (127)

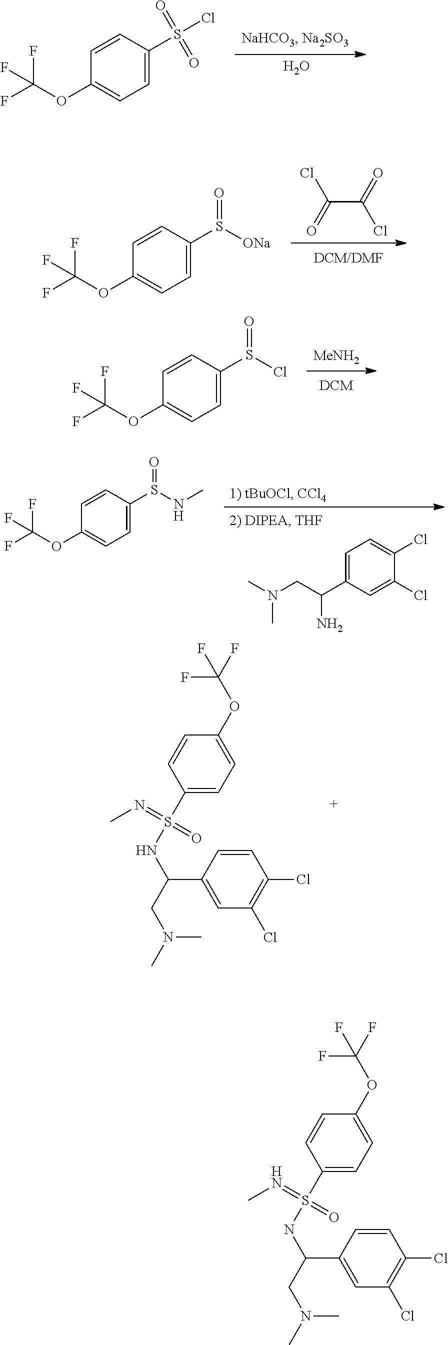

### Step 1: Synthesis of sodium 4-(trifluoromethoxy)benzenesulfinate

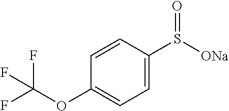

In a round bottomed flask equipped with a condenser, a solution of 4-(trifluoromethoxy)benzenesulfonyl chloride (5.00 g, 19.2 mmol), sodium hydrogen carbonate (3.55 g, 42.2 mmol), and disodium sulfite (5.32 g, 42.2 mmol) in water (50 mL) was stirred at 65° C. for 20 h. The mixture was allowed to cool to rt and was concentrated. Methanol (50 mL) was added to the residue and the suspension was stirred at rt for 1 h then filtered. The residue was washed with MeOH and the filtrate was concentrated to afford the title compound as a white powder (2.5 g, 52% yield, tᵣ=0.76 min). LCMS (Method F); ¹H-NMR (DMSO-d₆, 400 MHz) δ (ppm) 7.63-7.53 (m, 2H), 7.36-7.23 (m, 2H).

### Step 2: Synthesis of 4-(trifluoromethoxy)benzenesulfinyl chloride

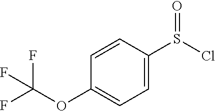 [SMILES: O=S(Cl)c1ccc(OC(F)(F)F)cc1]

In a round-bottomed flask, to a stirred suspension of sodium 4-(trifluoromethoxy)benzenesulfinate (2.88 g, 11.6 mmol) in anhydrous DCM (28.916 mL) at room temperature under nitrogen were added successively oxalyl chloride (1.5 mL, 17.4 mmol) and anhydrous DMF (0.1157 mL). The reaction mixture was stirred at room temperature for 2 h. The reaction mixture was concentrated under reduced pressure and dried under vacuum to afford the title compound as a beige powder (2.37 g, 67% yield, tᵣ=0.85 min). LCMS (Method E). The compound was use in the next step without further purification.

### Step 3: Synthesis of N-methyl-4-(trifluoromethoxy)benzenesulfinamide

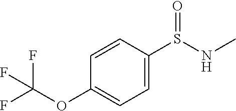

In a round-bottomed flask, to a stirred suspension of 4-(trifluoromethoxy)benzenesulfinyl chloride (100%, 2.37 g, 9.69 mmol) in anhydrous DCM (24.156 mL) at room temperature under nitrogen were added successively 2 M methylamine in THF (7.3 mL, 14.5 mmol) and triethylamine (4.1 mL, 29.1 mmol). The reaction mixture was stirred at room temperature for 20 h. Water (100 mL) was added, and the aqueous layer was extracted with dichloromethane (1×20 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5% to afford the title compound as a yellow oil (113 mg, 5% yield, tᵣ=0.74 min). LCMS (Method F): m/z found 240.1 [M+H]⁺; ¹H-NMR (DMSO-d₆, 400 MHz): δ (ppm) 7.72 (d, J=8.7 Hz, 2H), 7.56 (d, J=8.5 Hz, 2H), 6.74-6.53 (m, 1H), 2.26 (d, J=5.1 Hz, 3H).

### Step 4: Synthesis of N-(1-(3,4-dichlorophenyl)-2-(dimethylamino)ethyl)-N′-methyl-4-(trifluoromethoxy)benzenesulfonimidamide (126) and N′-(1-(3,4-dichlorophenyl)-2-(dimethylamino)ethyl)-N-methyl-4-(trifluoromethoxy)benzenesulfonimidamide (127)

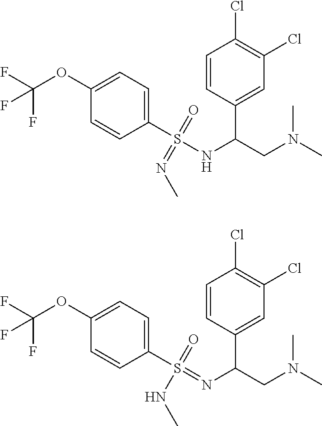 [SMILES: CN=S(=O)(NC(CN(C)C)c1ccc(Cl)c(Cl)c1)c1ccc(OC(F)(F)F)cc1 ; CNS(=O)(=NC(CN(C)C)c1ccc(Cl)c(Cl)c1)c1ccc(OC(F)(F)F)cc1]

In a round-bottomed flask, to a stirred solution of N-methyl-4-(trifluoromethoxy)benzenesulfinamide (48 mg, 0.201 mmol) in tetrachloromethane (0.84 mL, 8.67 mmol) at 0° C. under nitrogen was added tert-butyl hypochlorite (23 mg, 0.211 mmol). The reaction mixture was stirred at 0° C. for 1 h in the dark. The reaction mixture was concentrated under reduced pressure at 2° C. The residue was dissolved in anhydrous THF (0.6277 mL), then 1-(3,4-dichlorophenyl)-N²,N²-dimethylethane-1,2-diamine (51 mg, 0.221 mmol) and DIPEA (105 μL, 0.602 mmol) were added successively at 0° C. The reaction mixture was stirred at room temperature for 18 h. The reaction mixture was quenched with water (10 mL) and ethyl acetate (10 mL) was added. The aqueous layer was extracted with ethyl acetate (1×10 mL). The combined organic layers were washed with a saturated aqueous solution of sodium chloride, dried over sodium sulfate, filtered and concentrated under reduced pressure. The crude product was purified by flash chromatography on silica gel using a gradient of methanol in dichloromethane from 0% to 5%, and two different fractions were collected. The two residues were triturated with 2 M hydrogen chloride in diethyl ether (1.0 mL, 2.01 mmol), filtered, washed with diethyl ether and pentane, and dried under vacuum at 60° C. for 18 h to afford the title compounds as white powders. N-(1-(3,4-dichlorophenyl)-2-(dimethylamino)ethyl)-N′-methyl-4-(trifluoromethoxy)benzenesulfonimidamide hydrochloride (126) (11.3 mg, 11% yield, 98.4% purity, tᵣ=1.63 min). LCMS (Method C): m/z found 470 [M+H]⁺; ¹H-NMR (500 MHz, DMSO-d₆) δ (ppm) 8.80-9.46 (m, 1H) 8.01 (d, J=8.80 Hz, 2H) 7.62-7.71 (m, 1H) 7.59-7.61 (m, 1H) 7.53-7.56 (m, 1H) 7.49-7.53 (m, 2H) 7.27-7.39 (m, 1H) 4.79-5.01 (m, 1H) 3.20-3.30 (m, 2H) 2.71-2.97 (m, 6H) 2.43 (s, 3H). N′-(1-(3,4-dichlorophenyl)-2-(dimethylamino)ethyl)-N-methyl-4-(trifluoromethoxy)benzenesulfonimidamide (127) (12.9 mg, 12% yield, 95.2% purity, tᵣ=1.64 min). LCMS (Method C): m/z found 470 [M+H]⁺; ¹H-NMR (500 MHz, DMSO-d₆) δ ppm 8.39-9.04 (m, 1H) 8.09 (d, J=8.80 Hz, 2H) 7.75-7.83 (m, 1H) 7.66 (s, 3H) 7.48-7.57 (m, 1H) 6.92 (br d, J=5.14 Hz, 1H) 4.72-4.97 (m, 1H) 3.14-3.29 (m, 2H) 2.70-2.97 (m, 6H) 2.03-2.16 (m, 3H).

### Example 17: N-(1-(3,4-dichlorophenyl)-2-(dimethylamino)ethyl)-3-(trifluoromethoxy)benzenesulfonamide (128)

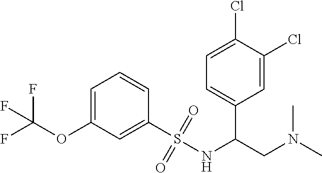 [SMILES: CN(C)CC(NS(=O)(=O)c1cccc(OC(F)(F)F)c1)c1ccc(Cl)c(Cl)c1]

In a sealed tube, a solution of 1-(3,4-dichlorophenyl)-N²,N²-dimethylethane-1,2-diamine (95%, 49 mg, 0.200 mmol) in DCM (0.25 mL) was added to a stirred solution of triethylamine (102 uL, 0.729 mmol) and 3-(trifluoromethoxy)benzenesulfonyl chloride (95%, 50 mg, 0.182 mmol) in DCM (1.2 mL). The solution was stirred at rt for 2.5 h and was diluted with water. The aqueous layer was extracted twice with DCM, the combined organic layers were washed with a saturated aqueous solution of NH₄Cl, a saturated aqueous solution of NaHCO₃, a saturated aqueous solution of NaCl, and dried using a phase separator and concentrated. The crude was purified by flash chromatography on silica gel using a gradient of MeOH in DCM from 0.2% to 5%. The desired fraction were concentrated. The residue was solubilized in 1 mL of Et₂O and HCl (200 μL, 2 N in Et₂O) was added. The mixture was stirred for 2 h, filtered under N₂, washed with Et₂O and dried under vacuum at 40° C. for 3 days to afford the title compound as a white powder (72.5 mg, 80% yield, 99.3% purity, tᵣ=1.54 min). LCMS (Method C): m/z found 456.9 [M+H]⁺; ¹H-NMR (600 MHz, DMSO-d₆) δ (ppm) 9.50-10.00 (m, 1H) 8.85-9.23 (m, 1H) 7.59-7.63 (m, 1H) 7.50-7.54 (m, 1H) 7.45-7.49 (m, 2H) 7.37-7.41 (m, 1H) 7.35 (d, J=1.00 Hz, 1H) 7.15-7.24 (m, 1H) 4.54-5.21 (m, 1H) 3.38-3.52 (m, 1H) 3.17-3.27 (m, 1H) 2.66-3.00 (m, 6H).

### Example 18: N-(1-(3,4-dichlorophenyl)-2-(dimethylamino)ethyl)-3-fluorobenzenesulfonamide (129)

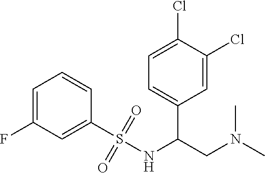 [SMILES: CN(C)CC(NS(=O)(=O)c1cccc(F)c1)c1ccc(Cl)c(Cl)c1]

In a sealed tube, 3-fluorobenzenesulfonyl chloride (97%, 25 μL, 0.183 mmol) was added to a stirred solution of 1-(3,4-dichlorophenyl)-N²,N²-dimethylethane-1,2-diamine (95%, 50 mg, 0.204 mmol) and triethylamine (102 μL, 0.733 mmol) in anhydrous DCM (2 mL). The solution was stirred at rt for 20 h. The crude was concentrated in vacuum, and purified by flash chromatography on silica gel using a gradient of methanol n dichloromethane from 0% to 5%. The crude was purified by reverse-phase chromatography using a gradient of acetonitrile in water from 0% to 100% (0.1% AcOH in water). The residue was triturated in pentane, filtered, washed with pentane and dried under vacuum at 50° C. for 24 h to afford the title compound as a white powder (34 mg, 47.4% yield, 100% purity, tᵣ=1.24 min). LCMS (Method C): m/z found 391 [M+H]⁺; ¹H-NMR (600 MHz, DMSO-d₆) δ (ppm) 8.30 (br s, 1H), 7.34-7.48 (m, 6H), 7.17 (dd, J=2.05, 8.36 Hz, 1H), 4.37 (t, J=7.48 Hz, 1H), 2.40 (dd, J=8.29, 12.54 Hz, 1H), 2.30 (dd, J=6.90, 12.62 Hz, 1H), 2.05 (s, 6H).

### Example 19: N-(1-(3,4-dichlorophenyl)-2-(dimethylamino)ethyl)-4-fluorobenzenesulfonamide (130)

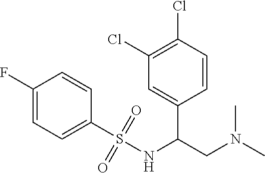 [SMILES: CN(C)CC(NS(=O)(=O)c1ccc(F)cc1)c1ccc(Cl)c(Cl)c1]

In a sealed tube, 4-fluorobenzenesulfonyl chloride (98%, 40 mg, 0.204 mmol) was added to a stirred solution of 1-(3,4-dichlorophenyl)-N²,N²-dimethylethane-1,2-diamine (95%, 50 mg, 0.204 mmol) and triethylamine (85 μL, 0.611 mmol) in anhydrous DCM (2.2 mL). The solution was stirred at room temperature for 20 h. Water (5 mL) was added and the aqueous layer was extracted with dichloromethane (1×5 mL). The combined organic layers were washed with a saturated aqueous solution of sodium chloride, dried over sodium sulfate, filtered and concentrated under reduced pressure. The crude product was purified by flash chromatography on silica gel using a gradient of methanol in dichloromethane from 0% to 5%. The residue was triturated in pentane, filtered, washed with pentane, and dried under vacuum at 50° C. for 18 h to afford the title compound as a white powder (39.9 mg, 50% yield, 99.9% purity, tᵣ=1.24 min). LCMS (Method C): m/z found 390.9 [M+H]⁺; ¹H-NMR (500 MHz, DMSO-d₆) δ (ppm) 8.18 (d, J=1.00 Hz, 1H) 7.60-7.70 (m, 2H) 7.41 (d, J=8.31 Hz, 1H) 7.34 (d, J=1.96 Hz, 1H) 7.19-7.27 (m, 2H) 7.14-7.19 (m, 1H) 4.18-4.42 (m, 1H) 2.25-2.44 (m, 2H) 2.04 (s, 6H).

### Example 20: N-(1-(3,4-dichlorophenyl)-2-(dimethylamino)ethyl)-2-methylbenzenesulfonamide (131)

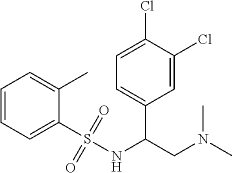 [SMILES: Cc1ccccc1S(=O)(=O)NC(CN(C)C)c1ccc(Cl)c(Cl)c1]

In a sealed tube, a solution of 2-methylbenzenesulfonyl chloride (35 mg, 0.184 mmol) in DCM (0.25 mL) was added to a stirred solution of 1-(3,4-dichlorophenyl)-N²,N²-dimethylethane-1,2-diamine (95%, 50 mg, 0.202 mmol) and triethylamine (102 μL, 0.734 mmol) in DCM (1.2 mL). The solution was stirred at rt for 2.5 h and was diluted with water. The aqueous layer was extracted twice with DCM, the combined organic layers were washed with a saturated aqueous solution of NH₄Cl, a saturated aqueous solution of NaHCO₃, a saturated aqueous solution of NaCl, and dried using a phase separator and concentrated. The crude was purified by flash chromatography on silica gel using a gradient of MeOH in DCM from 0.2% to 5%. The desired fraction were concentrated. The residue was triturated in pentane, filtered, washed with pentane, and dried under vacuum at 40° C. for 2 days to afford the title compound as a white powder (49.8 mg, 69% yield, 99% purity, tᵣ=1.33 min). LCMS (Method C): m/z found 387 [M+H]⁺; ¹H-NMR (500 MHz, DMSO-d₆) δ (ppm) 7.95-8.25 (m, 1H) 7.64-7.84 (m, 1H) 7.35-7.49 (m, 3H) 7.20-7.29 (m, 2H) 7.13-7.19 (m, 1H) 4.05-4.33 (m, 1H) 2.51-2.53 (m, 3H) 2.21-2.46 (m, 2H) 1.98 (s, 6H).

### Example 21: N-(1-(3,4-dichlorophenyl)-2-(dimethylamino)ethyl)-3-methylbenzenesulfonamide (132)

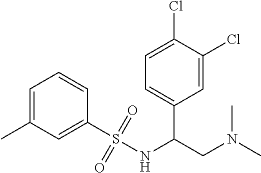

In a sealed tube, a solution of 1-(3,4-dichlorophenyl)-N²,N²-dimethylethane-1,2-diamine (95%, 49 mg, 0.198 mmol) in DCM (0.2468 mL) was added to a stirred solution of triethylamine (100 μL, 0.720 mmol) and 3-methylbenzenesulfonyl chloride (98%, 0.027 mL, 0.180 mmol) in DCM (1.1846 mL). The solution was stirred at rt for 2.5 h and was diluted with water. The aqueous layer was extracted twice with DCM, the combined organic layers were washed with a saturated aqueous solution of NH₄Cl, a saturated aqueous solution of NaHCO₃ and a saturated aqueous solution of NaCl, dried using a phase separator and concentrated. The crude was purified by flash chromatography on silica gel using a gradient of MeOH in DCM from 0.2% to 5%. The desired fractions were concentrated. The residue was triturated in pentane, filtered, washed with pentane and dried under vacuum at 40° C. for 3 days to afford the title product as a white powder (37.1 mg, 53% yield, 99.7% purity, tᵣ=1.36 min). LCMS (Method C): m/z found 386.9 [M+H]⁺; ¹H-NMR (600 MHz, DMSO-d₆) δ (ppm) 8.06 (d, J=1.00 Hz, 1H) 7.39-7.43 (m, 1H) 7.37 (d, J=8.22 Hz, 1H) 7.33 (d, J=2.05 Hz, 1H) 7.30-7.32 (m, 1H) 7.26-7.29 (m, 2H) 7.09-7.15 (m, 1H) 4.17-4.42 (m, 1H) 2.27-2.41 (m, 2H) 2.24 (s, 3H) 2.05 (s, 6H).

### Example 22: N-(1-(3,4-dichlorophenyl)-2-(dimethylamino)ethyl)-4-methylbenzenesulfonamide (133)

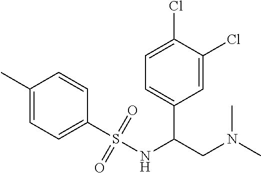

In a sealed tube, a solution of 4-methylbenzenesulfonyl chloride (35 mg, 0.184 mmol) in DCM (0.25 mL) was added to a stirred solution of 1-(3,4-dichlorophenyl)-N²,N²-dimethylethane-1,2-diamine (95%, 50 mg, 0.202 mmol) and triethylamine (102 μL, 0.734 mmol) in DCM (1.2 mL). The solution was stirred at rt for 2.5 h and was diluted with water. The aqueous layer was extracted twice with DCM, the combined organic layers were washed with a saturated aqueous solution of NH₄Cl, a saturated aqueous solution of NaHCO₃ and a saturated aqueous solution of NaCl, dried using a phase separator and concentrated. The crude was purified by flash chromatography on silica gel using a gradient of MeOH in DCM from 0.2% to 5%. The desired fractions were concentrated. The residue was triturated in pentane, filtered, washed with pentane and dried under vacuum at 40° C. for 2 days to afford the title compound as a white powder (45.4 mg, 64% yield, 99.8% purity, tᵣ=1.34 min). LCMS (Method C): m/z found 386.0 [M+H]⁺; ¹H-NMR (500 MHz, DMSO-d₆, 300K) δ (ppm) 7.98 (br d, J=7.3 Hz, 1H), 7.46 (d, J=8.3 Hz, 2H), 7.38 (d, J=8.3 Hz, 1H), 7.28 (d, J=2.0 Hz, 1H), 7.18 (d, J=8.1 Hz, 2H), 7.14 (dd, J=8.3, 2.0 Hz, 1H), 4.28 (br d, J=6.8 Hz, 1H), 2.26-2.40 (m, 5H), 2.04 (s, 6H).

### Example 23: N-(1-(3,4-dichlorophenyl)-2-(dimethylamino)ethyl)-3,5-difluorobenzenesulfonamide (134)

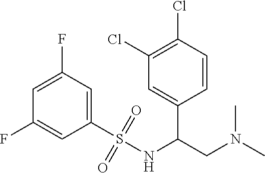 [SMILES: CN(C)CC(NS(=O)(=O)c1cc(F)cc(F)c1)c1ccc(Cl)c(Cl)c1]

In a sealed vial under nitrogen, 3,5-Difluorobenzenesulfonyl chloride (46 mg, 0.214 mmol) was added to a stirred solution of 1-(3,4-dichlorophenyl)-N²,N²-dimethylethane-1,2-diamine (50 mg, 0.214 mmol) and triethylamine (0.12 mL, 0.858 mmol) in anhydrous DCM (2.1446 mL). The solution was stirred at rt for 20 h, then concentrated in vacuo and purified by flash chromatography on silica gel using a gradient of MeOH (0.7N NH₃) in DCM from 0.4% to 6%. Desired fraction were gathered, concentrated in vacuo, sonicated in Et₂O, pentane was added and the suspension was filtered to afford the title compound as a white powder (38.6 mg, 44% yield, 100% purity, tᵣ=1.32 min). LCMS (Method C): m/z found 409.0 [M+H]⁺; ¹H-NMR (DMSO-d₆, 600 MHz): δ (ppm) 8.32-8.64 (m, 1H), 7.44-7.49 (m, 1H), 7.44 (d, J=8.2 Hz, 1H), 7.40 (d, J=1.9 Hz, 1H), 7.22-7.27 (m, 2H), 7.19 (dd, J=8.3, 2.0 Hz, 1H), 4.42 (t, J=7.3 Hz, 1H), 2.42-2.48 (m, 1H), 2.31 (br dd, J=12.2, 6.5 Hz, 1H), 2.08 (s, 6H).

### Example 24: N-(1-(3,4-dichlorophenyl)-2-(dimethylamino)ethyl)cyclohexanesulfonamide (135)

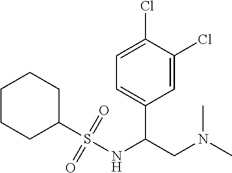 [SMILES: CN(C)CC(NS(=O)(=O)C1CCCCC1)c1ccc(Cl)c(Cl)c1]

In a sealed tube, cyclohexanesulfonyl chloride (153 μL, 1.29 mmol) was added to a stirred solution of 1-(3,4-dichlorophenyl)-N²,N²-dimethylethane-1,2-diamine (150 mg, 0.643 mmol) and pyridine (208 μL, 2.57 mmol) in THF (2 mL). The solution was stirred at rt for 18 h and was diluted with an half saturated aqueous solution of NaHCO₃. The aqueous layer was extracted with DCM, the combined organic layers were washed with a saturated aqueous solution of NaHCO₃, brine, dried over sodium sulfate, filtered and concentrated. The crude was purified by flash chromatography on silica gel using a gradient of (MeOH+2% NH₄OH) in DCM from 0.2% to 10%. to afford two pure fractions and an impure one. Each pure fraction was dissolved in Et₂O and added dropwise to stirred 2 N HCl/Et₂O. Each obtained suspension was stirred at rt for 16 h and filtered. Each residue was washed with Et₂O and dried under reduced pressure at 45° C. for 16 h to afford the salified starting amine. The impure fraction was purified again by flash chromatography on silica gel using a gradient of (MeOH+2% NH₄OH) in DCM from 0.2% to 10% to afford no pure fractions. The impure fractions were combined and concentrated then dissolved in Et₂O. This solution was added to stirred 2 N HCl/Et₂O and the suspension was stirred at rt for 3 h. The suspension was concentrated and dissolved in DCM. This solution was washed with saturated aqueous NaHCO₃, dried over sodium sulfate, filtered, concentrated and purified by flash chromatography on silica gel using a gradient of (MeOH+2% NH₄OH) in DCM from 0.2% to 5%. The desired fractions were concentrated and dissolved in Et₂O. The solution was added to stirred 2 N HCl/Et₂O and the suspension was stirred at rt for 16 h. The suspension was filtered, the residue was washed with Et₂O and dried under reduced pressure at 45° C. for 16 h to afford the title compound as a white powder (9 mg, 3% yield, 99.2% purity, tᵣ=1.75 min). LCMS (Method C): m/z found 379.0 [M+H]⁺; ¹H-NMR (DMSO-d₆, 500 MHz): δ (ppm) 9.42 (br d, J=2.4 Hz, 1H), 8.19 (br d, J=9.5 Hz, 1H), 7.95 (s, 1H), 7.74 (d, J=8.3 Hz, 1H), 7.61 (br d, J=8.1 Hz, 1H), 4.75-4.89 (m, 1H), 3.38-3.49 (m, 1H), 3.32-3.35 (m, 1H), 2.79-2.95 (m, 6H), 2.33-2.43 (m, 1H), 1.96 (br d, J=12.2 Hz, 1H), 1.72 (br d, J=13.0 Hz, 1H), 1.60 (br d, J=11.0 Hz, 2H), 1.51 (br d, J=12.2 Hz, 1H), 1.23-1.36 (m, 1H), 0.95-1.19 (m, 3H), 0.63-0.76 (m, 1H).

### Example 25: N-(1-(3,4-dichlorophenyl)-2-(dimethylamino)ethyl)-2-phenylethane-1-sulfonamide (136)

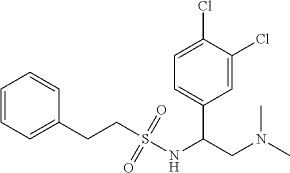 [SMILES: CN(C)CC(NS(=O)(=O)CCc1ccccc1)c1ccc(Cl)c(Cl)c1]

In a sealed tube, 2-phenylethanesulfonyl chloride (176 mg, 0.858 mmol) was added to a stirred solution of 1-(3,4-dichlorophenyl)-N²,N²-dimethylethane-1,2-diamine (100 mg, 0.429 mmol) and pyridine (138 μL, 1.72 mmol) in THF (2 mL). The solution was stirred at rt for 18 h and was diluted with an half saturated aqueous solution of NaHCO₃. The aqueous layer was extracted with DCM, the combined organic layers were washed with brine, dried over sodium sulfate, filtered and concentrated. The crude was purified by flash chromatography on silica gel using a gradient of (MeOH+2% NH₄OH) in DCM from 0.2% to 5%. The desired fractions were concentrated and purified again by flash chromatography on silica gel using a gradient of (MeOH+2% NH₄OH) in DCM from 0.2% to 5%. The desired fractions were concentrated and dissolved in Et₂O. The solution was added to stirred 2 N HCl/Et₂O and the obtained suspension was stirred at rt for 16 h and filtered. The residue was washed with Et₂O and dried under reduced pressure at 45° C. for 16 h to afford the title compound as a white powder (59 mg, 31% yield, 99.9% purity, tᵣ=1.39 min). LCMS (Method C): m/z found 401.3 [M+H]⁺; ¹H-NMR (500 MHz, DMSO-d₆, 300 K) δ (ppm) 9.63 (br s, 1H), 8.47 (br d, J=9.5 Hz, 1H), 8.00 (d, J=1.2 Hz, 1H), 7.73 (d, J=8.3 Hz, 1H), 7.65 (dd, J=1.8, 8.4 Hz, 1H), 7.31-7.07 (m, 3H), 6.95-6.78 (m, 2H), 4.93 (dt, J=4.0, 9.8 Hz, 1H), 3.59-3.40 (m, 1H), 3.30-3.22 (m, 1H), 2.97-2.76 (m, 8H), 2.67-2.57 (m, 1H), 2.69-2.55 (m, 1H).

### Example 26: N-(1-(3,4-dichlorophenyl)-2-(dimethylamino)ethyl)-4-propylbenzenesulfonamide (137)

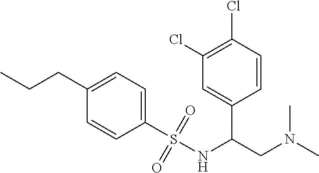 [SMILES: CCCc1ccc(S(=O)(=O)NC(CN(C)C)c2ccc(Cl)c(Cl)c2)cc1]

In a sealed tube, 4-propylbenzenesulfonyl chloride (29 μL, 0.163 mmol) was added to a stirred solution of 1-(3,4-dichlorophenyl)-N²,N²-dimethylethane-1,2-diamine (95%, 40 mg, 0.163 mmol) and triethylamine (68 μL, 0.489 mmol) in anhydrous DCM (1.76 mL). The solution was stirred at room temperature for 20 h. Water (5 mL) was added and the aqueous layer was extracted with dichloromethane (1×5 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was triturated in 2 M hydrogen chloride in diethyl ether (0.81 mL, 1.63 mmol), filtered, washed with diethyl ether and dried under vacuum at 60° C. for 18 h to afford the title compound as a white powder (29.1 mg, 39% yield, 97.9% purity, tᵣ=1.63 min). LCMS (Method C): m/z found 415.0 [M+H]⁺; ¹H-NMR (500 MHz, DMSO-d₆) δ (ppm) 9.41-9.81 (m, 1H) 8.44-8.92 (m, 1H) 7.40-7.47 (m, 3H) 7.34 (d, J=8.31 Hz, 1H) 7.21 (dd, J=8.31, 2.20 Hz, 1H) 7.10 (d, J=8.31 Hz, 2H) 4.51-5.17 (m, 1H) 3.32-3.40 (m, 1H) 3.14-3.25 (m, 1H) 2.74-2.96 (m, 6H) 2.47-2.50 (m, 2H) 1.50 (d, J=7.58 Hz, 2H) 0.84 (t, J=7.34 Hz, 3H).

### Example 27: N-(1-(3,4-dichlorophenyl)-2-(dimethylamino)ethyl)-4-phenoxybenzenesulfonamide (138)

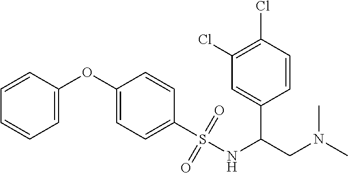 [SMILES: CN(C)CC(NS(=O)(=O)c1ccc(Oc2ccccc2)cc1)c1ccc(Cl)c(Cl)c1]

In a sealed tube, 4-phenoxybenzenesulfonyl chloride (55 mg, 0.204 mmol) was added to a stirred solution of 1-(3,4-dichlorophenyl)-N²,N²-dimethylethane-1,2-diamine (95%, 50 mg, 0.204 mmol) and triethylamine (85 μL, 0.611 mmol) in anhydrous DCM (2.2 mL). The solution was stirred at room temperature for 20 h. Water (5 mL) was added and the aqueous layer was extracted with dichloromethane (1×5 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was triturated in pentane, filtered, washed with pentane and dried under vacuum at 60° C. for 18 h to afford the title compound as a white powder (48.1 mg, 51% yield, 99.9% purity, tᵣ=1.66 min). LCMS (Method C): m/z found 465 [M+H]⁺; ¹H-NMR (500 MHz, DMSO-d₆) δ (ppm) 8.09 (br d, J=1.47 Hz, 1H) 7.56 (d, J=8.80 Hz, 2H) 7.47 (dd, J=8.44, 7.46 Hz, 2H) 7.42 (d, J=8.31 Hz, 1H) 7.35 (d, J=1.96 Hz, 1H) 7.22-7.28 (m, 1H) 7.13-7.18 (m, 1H) 6.99-7.07 (m, 2H) 6.86-6.92 (m, 2H) 4.17-4.48 (m, 1H) 2.25-2.48 (m, 2H) 2.08 (br s, 6H).

### Example 28: N-(1-(3,4-dichlorophenyl)-2-(dimethylamino)ethyl)-4-(4-(trifluoromethyl)phenoxy)benzenesulfonamide (139)

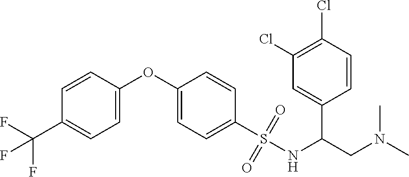 [SMILES: CN(C)CC(NS(=O)(=O)c1ccc(Oc2ccc(C(F)(F)F)cc2)cc1)c1ccc(Cl)c(Cl)c1]

In a sealed tube, 4-[4-(trifluoromethyl)phenoxy]benzenesulfonyl chloride (95%, 49 μL, 0.204 mmol) was added to a stirred solution of 1-(3,4-dichlorophenyl)-N²,N²-dimethylethane-1,2-diamine (95%, 50 mg, 0.204 mmol) and triethylamine (85 μL, 0.611 mmol) in anhydrous DCM (2.2 mL). The solution was stirred at room temperature for 18 h. Water (2 mL) was added and the aqueous layer was extracted with dichloromethane (2×5 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 4%. The residue was triturated in pentane, filtered, washed with pentane and dried under vacuum at 60° C. for 18 h to afford the title compound as a white powder (64.2 mg, 59% yield, 99.7% purity, tᵣ=1.89 min). LCMS (Method C): m/z found 533.0 [M+H]⁺; ¹H-NMR (500 MHz, DMSO-d₆) δ (ppm) 8.17 (br s, 1H), 7.82 (d, J=8.80 Hz, 2H), 7.62 (d, J=7.69 Hz, 2H), 7.45 (d, J=8.31 Hz, 1H), 7.38 (d, J=1.96 Hz, 1H), 7.16-7.20 (m, 3H), 7.05 (d, J=7.85 Hz, 2H), 4.36 (t, J=7.46 Hz, 1H), 2.42 (dd, J=8.07, 12.47 Hz, 1H), 2.31 (dd, J=6.97, 12.59 Hz, 1H), 2.08 (s, 6H).

### Example 29: N-(cyclohexyl(3,5-dichlorophenyl)methyl)-4-(trifluoromethoxy)benzenesulfonamide (140 and 141

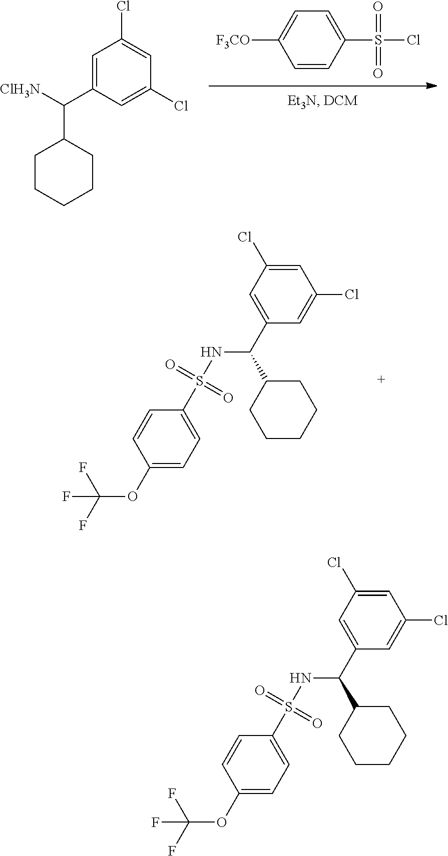

In a sealed tube, 4-(trifluoromethoxy)benzenesulfonyl chloride (602 μL, 3.39 mmol) was added to a stirred solution of cyclohexyl(3,5-dichlorophenyl)methanamine hydrochloride (95%, 1.05 g, 3.39 mmol) and triethylamine (2.4 mL, 17.0 mmol) in anhydrous DCM (35 mL). The solution was stirred at room temperature for 20 h. Water (50 mL) was added and the aqueous layer was extracted with dichloromethane (2×25 mL). The combined organic layers were washed with a saturated aqueous solution of sodium chloride, dried over sodium sulfate, filtered and concentrated under reduced pressure. The crude was purified by flash chromatography on silica gel using a gradient of ethyl acetate in cyclohexane from 10% to 30% to obtain the title compound as a racemic mixture. 980 mg of the racemic mixture was dissolved by sonication in EtOH (12 mL) to provide a solution having a concentration of 81.6 mg/mL. The solution was passed through a GHP filter (0.45 μm). 20 injections of the sample solution were performed on a Chiralpak IB column (250×20 mm, 5 μm) using 7% EtOH in CO₂ as a mobile phase (Waters Prep SFC80, flow rate=50 mL/min, T=40° C., P=100 bar, UV detection at 210 nm) to afford the separated enantiomers.

Enantiomer I (141): (396.8 mg, 24.2% yield, 100% purity, tᵣ=3.39 min). LCMS (Method C): m/z found 479.9 [M−H]⁻; ¹H-NMR (DMSO-d₆, 600 MHz): δ (ppm) 8.33 (br s, 1H), 7.61 (d, J=8.9 Hz, 2H), 7.27-7.35 (m, 2H), 7.18 (t, J=1.8 Hz, 1H), 7.09 (d, J=1.8 Hz, 2H), 4.03 (br d, J=8.5 Hz, 1H), 0.67-1.98 (m, 11H).

Enantiomer II (140): (351.3 mg, 22% yield, 100% purity, tᵣ=3.39 min). LCMS (Method C): m/z found 479.9 [M+H]⁺; ¹H-NMR (DMSO-d₆, 600 MHz): δ (ppm) 8.33 (br d, J=8.2 Hz, 1H), 7.56-7.67 (m, 2H), 7.25-7.34 (m, 2H), 7.18 (t, J=1.9 Hz, 1H), 7.09 (d, J=1.9 Hz, 2H), 4.03 (br t, J=8.4 Hz, 1H), 0.73-1.99 (m, 11H).

### Example 30: N-(1-(2-bromo-4-fluorophenyl)-2-cyclopropylethyl)-4-(trifluoromethoxy)benzenesulfonamide (142a and 142b

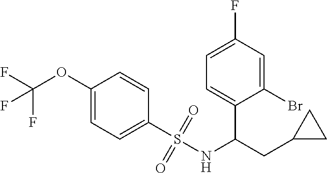 [SMILES: O=S(=O)(NC(CC1CC1)c1ccc(F)cc1Br)c1ccc(OC(F)(F)F)cc1]

In a sealed tube, a solution of 4-(trifluoromethoxy)benzenesulfonyl chloride (596 μL, 3.52 mmol) in DCM (4.4 mL) was added to a stirred solution of 1-(2-bromo-4-fluoro-phenyl)-2-cyclopropyl-ethanamine (1000 mg, 3.87 mmol) and triethylamine (2.0 mL, 14.1 mmol) in DCM (22 mL). The solution was stirred at rt for 20 h and was diluted with water. The aqueous layer was extracted with DCM, the combined organic layers were washed with a saturated aqueous solution of NH₄Cl, a saturated aqueous solution of NaHCO₃, dried over sodium sulfate, filtered and concentrated. The crude was purified by flash chromatography on silica gel using a gradient of (MeOH+2% NH₄OH) in DCM from 0.2% to 1% to afford the racemic mixture as a colorless oil. (1.5 g, 83% yield). 1.5 g of the racemic mixture was dissolved by sonication in EtOH (13 mL) and MeOH (2 mL) to provide a solution having a concentration of 100 mg/mL. The solution was filtered through a hydrophilic propylene (GHP) 0.45 μm filter. 150 injections of the sample solution was performed on a Chiralpak IB 5 μm, 250×20 mm stationary phase using 5% EtOH in CO₂ as a mobile phase (Waters Prep SFC80, flow rate=50 mL/min, T=40° C., P=100 bar, UV detection at 210 nm) to afford the title compounds as colorless oils

Enantiomer I (142a): (383.9 mg, 22% yield, 100% purity, tᵣ=3.05 min). LCMS (Method C): m/z found 479.9 [M−H]⁻; ¹H-NMR-d₆, 500 MHz) δ (ppm) 8.45 (s, 1H), 7.70 (d, J=8.8 Hz, 2H), 7.38 (d, J=8.3 Hz, 2H), 7.13-7.23 (m, 2H), 7.00 (dd, J=9.8, 1.5 Hz, 1H), 4.33 (dd, J=8.2, 6.5 Hz, 1H), 1.65 (ddd, J=14.2, 8.2, 6.2 Hz, 1H), 1.27 (dt, J=14.1, 7.0 Hz, 1H), 0.46-0.62 (m, 1H), 0.24-0.37 (m, 1H), 0.13 (dt, J=8.4, 4.3 Hz, 1H), 0.04-(−0.06) (m, 1H), −0.08 (dt, J=9.3, 4.6 Hz, 1H)

Enantiomer II (142b): (280 mg, 15% yield, 95.6% purity, tᵣ=3.05 min). LCMS (Method C): m/z found 479.8 [M−H]⁻; ¹H-NMR (DMSO-d₆, 500 MHz): δ (ppm) 8.46 (br s, 1H), 7.70 (d, J=8.8 Hz, 2H), 7.38 (d, J=8.3 Hz, 2H), 7.13-7.23 (m, 2H), 7.00 (dd, J=9.5, 1.5 Hz, 1H), 4.33 (dd, J=8.2, 6.5 Hz, 1H), 1.66 (ddd, J=14.1, 8.3, 6.2 Hz, 1H), 1.27 (dt, J=14.1, 7.0 Hz, 1H), 0.49-0.61 (m, 1H), 0.27-0.35 (m, 1H), 0.09-0.17 (m, 1H), 0.01 (dq, J=9.3, 4.7 Hz, 1H), −0.09 (dq, J=9.3, 4.7 Hz, 1H).

### Example 31: N-(1-(3,4-dichlorophenyl)-2-(dimethylamino)ethyl)-[1,1′-biphenyl]-4-sulfonamide (143)

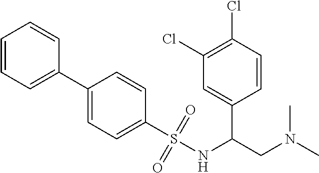 [SMILES: CN(C)CC(NS(=O)(=O)c1ccc(-c2ccccc2)cc1)c1ccc(Cl)c(Cl)c1]

In a sealed vial under nitrogen, biphenyl-4-sulfonyl chloride (97%, 56 mg, 0.214 mmol) was added to a stirred solution of 1-(3,4-dichlorophenyl)-N²,N²-dimethylethane-1,2-diamine (50 mg, 0.214 mmol) and triethylamine (0.12 mL, 0.858 mmol) in anhydrous DCM (2.1446 mL). The solution was stirred at rt for 20 h, then concentrated in vacuo and purified by flash chromatography on silica gel using a gradient of MeOH (0.7 N NH₃) in DCM from 0% to 5%. Desired fractions were gathered, concentrated in vacuo, sonicated in pentane and the suspension was filtered to afford the title compound as a white powder (47.7 mg, 49% yield, 99.45% purity, tᵣ=1.63 min). LCMS (Method C): m/z found 448 [M+H]⁺; ¹H-NMR (DMSO-d₆, 500 MHz): δ (ppm) 8.16 (br s, 1H), 7.68-7.68 (m, 1H), 7.59-7.69 (m, 5H), 7.47-7.53 (m, 2H), 7.40-7.45 (m, 1H), 7.37 (d, J=8.1 Hz, 1H), 7.33 (d, J=2.0 Hz, 1H), 7.16 (dd, J=8.3, 2.0 Hz, 1H), 4.36 (t, J=7.5 Hz, 1H), 2.29-2.47 (m, 2H), 2.06 (s, 6H).

### Example 32: N-(1-(3,4-dichlorophenyl)-2-(dimethylamino)ethyl)-[1,1′-biphenyl]-3-sulfonamide (144)

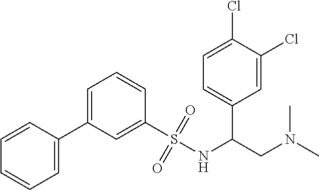 [SMILES: CN(C)CC(NS(=O)(=O)c1cccc(-c2ccccc2)c1)c1ccc(Cl)c(Cl)c1]

In a sealed tube, a solution of 1-(3,4-dichlorophenyl)-N²,N²-dimethylethane-1,2-diamine (95%, 51 mg, 0.207 mmol) in DCM (0.25 mL) was added to a stirred solution of triethylamine (105 μL, 0.752 mmol) and biphenyl-3-sulfonyl chloride (95%, 50 mg, 0.188 mmol) in DCM (1.2 mL). The solution was stirred at rt overnight and was diluted with water. The aqueous layer was extracted twice with DCM, the combined organic layers were washed with a saturated aqueous solution of NH₄Cl, a saturated aqueous solution of NaHCO₃ and a saturated aqueous solution of NaCl, dried using a phase separator and concentrated. The crude was purified by flash chromatography on silica gel using a gradient of MeOH in DCM from 0.2% to 5%. The desired fractions were concentrated. The residue was precipitated in a mixture of Et₂O and pentane, filtered, washed with pentane and dried under vacuum at 40° C. for 18 h to afford the title compound as a white powder (53.2 mg, 63% yield, 99.5% purity, tᵣ=1.64 min). LCMS (Method C): m/z found 449.0 [M+H]⁺; ¹H-NMR (DMSO-d₆, 500 MHz): δ (ppm) 8.22 (br s, 1H), 7.74-7.81 (m, 2H), 7.54-7.63 (m, 3H), 7.47-7.53 (m, 3H), 7.40-7.45 (m, 1H), 7.37 (d, J=2.2 Hz, 1H), 7.30 (d, J=8.3 Hz, 1H), 7.15 (dd, J=8.3, 2.0 Hz, 1H), 4.40 (t, J=7.5 Hz, 1H), 2.26-2.46 (m, 2H), 2.04 (s, 6H).

### Example 33: N-(1-(3,4-dichlorophenyl)-2-(dimethylamino)ethyl)naphthalene-1-sulfonamide (145)

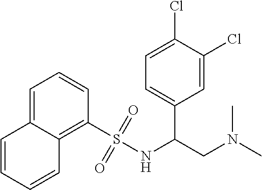 [SMILES: CN(C)CC(NS(=O)(=O)c1cccc2ccccc12)c1ccc(Cl)c(Cl)c1]

In a sealed tube, naphthalene-1-sulfonyl chloride (46 mg, 0.204 mmol) was added to a stirred solution of 1-(3,4-dichlorophenyl)-N²,N²-dimethylethane-1,2-diamine (95%, 50 mg, 0.204 mmol) and triethylamine (114 μL, 0.815 mmol) in anhydrous DCM (2.1429 mL). The solution was stirred at room temperature for 20 h. Water (10 mL) was added and the aqueous layer was extracted with dichloromethane (1×10 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was triturated in 2 M hydrogen chloride in diethyl ether (1.0 mL, 2.04 mmol), filtered, washed with diethyl ether and dried under vacuum at 60° C. for 20 h to afford the title compound as a white powder (69.1 mg, 73% yield, 99.1% purity, tᵣ=1.45 min). LCMS (Method C): m/z found 423.0 [M+H]⁺; ¹H-NMR (500 MHz, DMSO-d₆) in protonated form: δ (ppm) 9.64 (br s, 1H), 8.93 (br d, J=10.03 Hz, 1H), 8.43 (d, J=8.56 Hz, 1H), 8.01 (dd, J=4.89, 7.82 Hz, 2H), 7.87 (d, J=8.07 Hz, 1H), 7.65 (t, J=7.67 Hz, 1H), 7.58 (t, J=7.49 Hz, 1H), 7.45 (t, J=7.83 Hz, 1H), 7.07 (d, J=1.96 Hz, 1H), 6.76-6.83 (m, 2H), 4.63-4.73 (m, 1H), 3.36-3.51 (m, 1H), 3.06-3.27 (m, 1H), 2.70-2.99 (m, 6H).

### Example 34: N-(1-(3,4-dichlorophenyl)-2-(dimethylamino)ethyl)naphthalene-2-sulfonamide (146)

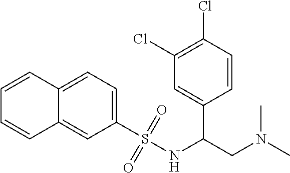 [SMILES: CN(C)CC(NS(=O)(=O)c1ccc2ccccc2c1)c1ccc(Cl)c(Cl)c1]

In a sealed vial under nitrogen, 2-naphthalenesulfonyl chloride (98%, 50 mg, 0.214 mmol) was added to a stirred solution of 1-(3,4-dichlorophenyl)-N²,N²-dimethylethane-1,2-diamine (50 mg, 0.214 mmol) and triethylamine (0.12 mL, 0.858 mmol) in anhydrous DCM (2.1446 mL). The solution was stirred at rt for 20 h, then concentrated in vacuo and purified by flash chromatography on silica gel using a gradient of MeOH (0.7 N NH₃) in DCM from 0.4% to 5%. Desired fractions were gathered, concentrated in vacuo, dissolved in DCM, pentane was added and the suspension was filtered to afford the title compound as a white powder (46.8 mg, 50% yield, 97.9% purity, tᵣ=1.48 min). LCMS (Method C): m/z found 422.9 [M+H]⁺; ¹H-NMR (DMSO-d₆, 500 MHz): δ (ppm) 8.17 (d, J=1.7 Hz, 2H), 7.56-8.01 (m, 6H), 7.30 (d, J=2.0 Hz, 1H), 7.22 (d, J=8.3 Hz, 1H), 7.12 (dd, J=8.3, 2.2 Hz, 1H), 4.38 (br t, J=7.6 Hz, 1H), 2.24-2.44 (m, 2H), 2.00 (s, 6H).

### Example 35: N-(1-(3,4-dichlorophenyl)-2-(dimethylamino)ethyl)-4-(trifluoromethoxy)benzenesulfonamide (148 and 149

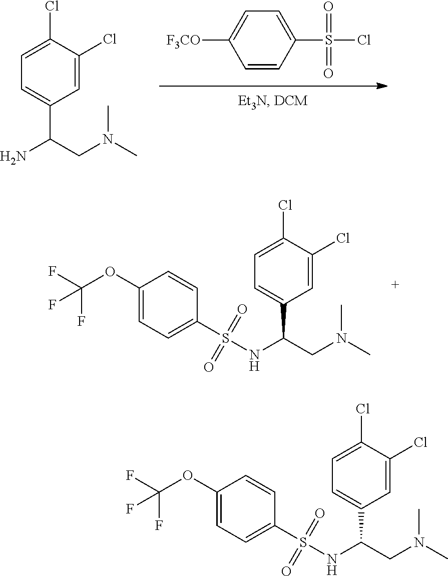 [SMILES: CN(C)CC(N)c1ccc(Cl)c(Cl)c1 ; O=S(=O)(Cl)c1ccc(OC(F)(F)F)cc1 ; CN(C)C[C@@H](NS(=O)(=O)c1ccc(OC(F)(F)F)cc1)c1ccc(Cl)c(Cl)c1 ; CN(C)C[C@H](NS(=O)(=O)c1ccc(OC(F)(F)F)cc1)c1ccc(Cl)c(Cl)c1]

In a sealed tube, 4-(trifluoromethoxy)benzenesulfonyl chloride (83 μL, 0.487 mmol) was added to a stirred solution of 1-(3,4-dichlorophenyl)-N′,N′-dimethyl-ethane-1,2-diamine (125 mg, 0.536 mmol) and triethylamine (272 μL, 1.95 mmol) in DCM (4.8741 mL). The solution was stirred at rt for 20 h. The crude was concentrated in vacuo and purified by flash chromatography on silica gel using a gradient of MeOH (0.7 N NH₃) in DCM from 0.4% to 5% twice to afford the expected racemic product (N-(1-(3,4-dichlorophenyl)-2-(dimethylamino)ethyl)-4-(trifluoromethoxy)benzenesulfonamide) as a pale yellow oil (126 mg, 55% yield). The sample was subjected to chiral separation to afford separated enantiomers. Each respective enantiomer was dissolved in dichloromethane, washed with a saturated NaHCO₃ solution, concentrated in vacuo, and triturated in pentane to afford a racemic mixture of the title compounds as a white powder. 120 mg of the racemic mixture was dissolved by sonication in 2 mL (MeOH+0.5% i-PrNH₂) and MeOH (2 mL) to provide a solution having a concentration of 30.0 mg/mL. The solution was passed through a GHP filter (0.45 μm). 20 injections of the sample solution were performed on a Chiralpak AD-H column (250×20 mm, 5 μm) using 10% (MeOH+0.5% i-PrNH₂) in CO₂ as a mobile phase (Waters Prep SFC80, flow rate=50 mL/min, T=40° C., P=143 bar, UV detection at 210 nm) to afford the separated enantiomers.

Enantiomer I (148): (19 mg, yield, 9% yield, 100% purity, tᵣ=1.54 min). LCMS (Method C): m/z found 457 [M+H]⁺; ¹H-NMR (600 MHz, DMSO-d₆) δ (ppm) 8.32 (br s, 1H), 7.71 (d, J=8.80 Hz, 2H), 7.34-7.39 (m, 4H), 7.15 (dd, J=2.05, 8.36 Hz, 1H), 4.36 (br s, 1H), 2.35-2.48 (m, 1H), 2.29 (br s, 1H), 2.04 (br s, 6H).

Enantiomer II (149): (29.1 mg, 13% yield, 100% purity, tᵣ=1.55 min). LCMS (Method C): m/z found 457 [M+H]⁺; ¹H-NMR (500 MHz, DMSO-d₆) δ (ppm) 8.33 (br s, 1H), 7.71 (d, J=7.91 Hz, 2H), 7.35-7.39 (m, 4H), 7.15 (dd, J=2.20, 8.31 Hz, 1H), 4.35 (br s, 1H), 2.33-2.47 (m, 1H), 2.17-2.33 (m, 1H), 2.04 (s, 6H).

### Example 36: N-(2-(dimethylamino)-1-(o-tolyl)ethyl)-4-(trifluoromethoxy)benzenesulfonamide (150)

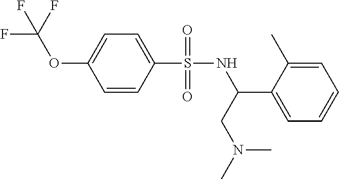 [SMILES: Cc1ccccc1C(CN(C)C)NS(=O)(=O)c1ccc(OC(F)(F)F)cc1]

In a sealed tube, a solution of 4-(trifluoromethoxy)benzenesulfonyl chloride (98%, 50 mg, 0.188 mmol) in DCM (0.25 mL) was added to a stirred solution of N′,N′-dimethyl-1-(o-tolyl)ethane-1,2-diamine;dihydrochloride (95%, 55 mg, 0.207 mmol) and triethylamine (131 μL, 0.940 mmol) in DCM (1.2 mL). The solution was stirred at rt overnight and was diluted with water. The aqueous layer was extracted with DCM, the combined organic layers were washed with a saturated aqueous solution of NH₄Cl, a saturated aqueous solution of NaHCO₃ and a saturated aqueous solution of NaCl, dried using a phase separator and concentrated. The crude was purified by flash chromatography on silica gel using a gradient of MeOH in DCM from 0.2% to 5%. The desired fractions were concentrated. The residue was triturated in a minimum of cold pentane, filtered, washed with cold pentane and dried under vacuum at 40° C. for 18 h to afford the title compound as a white powder (40.2 mg, 53% yield, 100% purity, tᵣ=1.38 min). LCMS (Method C): m/z 403 [M+H]⁺; ¹H-NMR (500 MHz, DMSO-d₆) δ (ppm) 8.28 (br s, 1H), 7.67 (d, J=7.98 Hz, 2H), 7.33 (d, J=8.07 Hz, 2H), 7.11 (d, J=7.58 Hz, 1H), 6.90-6.99 (m, 3H), 4.56 (br s, 1H), 2.44-2.49 (m, 1H), 2.22 (s, 3H), 2.11 (dd, J=4.89, 12.72 Hz, 1H), 2.04 (s, 6H).

### Example 37: 2-chloro-N-(1-(3,4-dichlorophenyl)-2-(dimethylamino)ethyl)-4-(trifluoromethoxy)benzenesulfonamide (151)

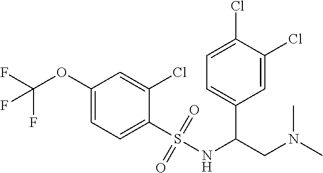 [SMILES: CN(C)CC(NS(=O)(=O)c1ccc(OC(F)(F)F)cc1Cl)c1ccc(Cl)c(Cl)c1]

In a sealed tube, 2-chloro-4-(trifluoromethoxy)benzenesulfonyl chloride (42 μL, 0.203 mmol) was added to a stirred solution of 1-(3,4-dichlorophenyl)-N²,N²-dimethylethane-1,2-diamine (52 mg, 0.224 mmol) and triethylamine (113 μL, 0.813 mmol) in DCM (1.2 mL). The solution was stirred at rt for 18 h and was diluted with an half saturated aqueous solution of NaHCO₃. The aqueous layer was extracted with DCM, the combined organic layers were washed with a saturated aqueous solution of NaHCO₃, brine, dried over sodium sulfate, filtered and concentrated. The crude was purified by flash chromatography on silica gel using a gradient of (MeOH+2% NH₄OH) in DCM from 0.2% to 5%. The desired fractions were concentrated and pentane was added to the residue. The obtained suspension was triturated and filtered. The residue was washed with pentane and was dried under reduced pressure at 40° C. for 16 h to afford the title compound as a white powder. (58 mg, 57% yield, 99.2% purity, tᵣ=1.78 min). LCMS (Method C): m/z found 490.3 [M+H]⁺; ¹H-NMR (500 MHz, DMSO-d₆, 300 K) δ (ppm) 8.69-8.25 (m, 1H), 8.00 (d, J=8.8 Hz, 1H), 7.56 (d, J=2.0 Hz, 1H), 7.45 (d, J=2.0 Hz, 1H), 7.44-7.37 (m, 2H), 7.18 (dd, J=2.0, 8.3 Hz, 1H), 4.31 (dd, J=6.2, 8.7 Hz, 1H), 2.54 (dd, J=8.8, 12.5 Hz, 1H), 2.29 (dd, J=6.2, 12.6 Hz, 1H), 2.02 (s, 6H).

### Example 38: 3-chloro-N-(1-(3,4-dichlorophenyl)-2-(dimethylamino)ethyl)-4-(trifluoromethoxy)benzenesulfonamide (152)

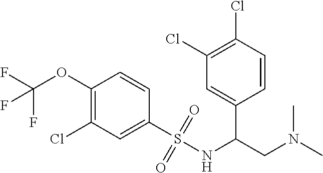 [SMILES: CN(C)CC(NS(=O)(=O)c1ccc(OC(F)(F)F)c(Cl)c1)c1ccc(Cl)c(Cl)c1]

In a sealed vial under nitrogen, 3-chloro-4-(trifluoromethoxy)benzenesulfonyl chloride (98%, 65 mg, 0.216 mmol) was added to a stirred solution of 1-(3,4-dichlorophenyl)-N²,N²-dimethylethane-1,2-diamine (50 mg, 0.216 mmol) and triethylamine (0.12 mL, 0.864 mmol) in anhydrous DCM (2.1589 mL). The solution was stirred at rt for 20 h, then concentrated in vacuo and purified by flash chromatography on silica gel using a gradient of MeOH in DCM from 0% to 8%. The desired fractions were gathered, concentrated in vacuo, sonicated in Et₂O and evaporated to afford the title compound as a white powder (66 mg, 62% yield, 99.1% yield, tᵣ=1.69 min). LCMS (Method C): m/z found 490.9 [M+H]⁺; ¹H-NMR (500 MHz, DMSO-d₆) δ (ppm) 8.50 (br s, 1H), 7.74 (d, J=2.20 Hz, 1H), 7.64-7.67 (m, 1H), 7.59-7.62 (m, 1H), 7.37-7.40 (m, 2H), 7.16 (dd, J=2.20, 8.31 Hz, 1H), 4.43 (dd, J=6.36, 8.56 Hz, 1H), 2.45-2.48 (m, 1H), 2.28 (dd, J=6.48, 12.59 Hz, 1H), 2.08 (s, 6H).

### Example 39: N-(1-(3,4-dichlorophenyl)-2-(dimethylamino)ethyl)-4-(difluoromethoxy)benzenesulfonamide (153)

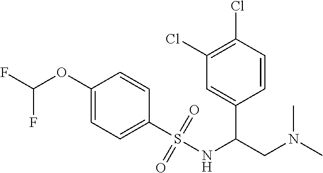 [SMILES: CN(C)CC(NS(=O)(=O)c1ccc(OC(F)F)cc1)c1ccc(Cl)c(Cl)c1]

In a sealed vial under nitrogen, 4-(difluoromethoxy)benzenesulfonyl chloride (95%, 0.036 mL, 0.214 mmol) was added to a stirred solution of 1-(3,4-dichlorophenyl)-N²,N²-dimethylethane-1,2-diamine (50 mg, 0.214 mmol) and triethylamine (0.12 mL, 0.858 mmol) in anhydrous DCM (2.1446 mL). The solution was stirred at rt for 72 h, concentrated in vacuo and purified by flash chromatography on silica gel using a gradient of MeOH (0.7 N NH₃) in DCM from 0.4% to 2%. Desired fractions were gathered, concentrated in vacuo, sonicated in Et₂O and filtered to afford the title compound as a white powder (62.2 mg, 65% yield, 99.2% purity, tᵣ=1.31 min). LCMS (Method C): m/z found 439.2 [M+H]⁺; ¹H-NMR (DMSO-d₆, 500 MHz): δ (ppm) 8.17 (br s, 1H), 7.62-7.68 (m, 2H), 7.41 (d, J=8.3 Hz, 1H), 7.35 (d, J=2.0 Hz, 1H), 7.14-7.19 (m, 3H), 7.29 (t, J=73.4 Hz, 1H), 4.32 (t, J=7.6 Hz, 1H), 2.38 (dd, J=12.6, 8.2 Hz, 1H), 2.29 (dd, J=12.5, 7.1 Hz, 1H), 2.04 (s, 6H).

### Example 40: 4-cyclopropoxy-N-(1-(3,4-dichlorophenyl)-2-(dimethylamino)ethyl)benzenesulfonamide (154)

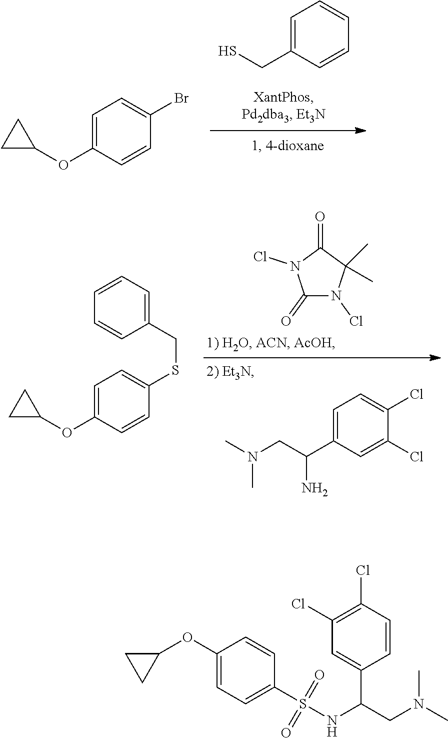 [SMILES: SCc1ccccc1 ; Brc1ccc(OC2CC2)cc1 ; c1ccc(CSc2ccc(OC3CC3)cc2)cc1 ; CN(C)CC(N)c1ccc(Cl)c(Cl)c1 ; CC1(C)C(=O)N(Cl)C(=O)N1Cl ; CN(C)CC(NS(=O)(=O)c1ccc(OC2CC2)cc1)c1ccc(Cl)c(Cl)c1]

### Step 1: Synthesis of 1-benzylsulfanyl-4-(cyclopropoxy)benzene

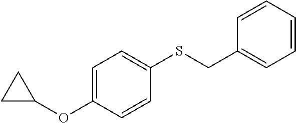 [SMILES: c1ccc(CSc2ccc(OC3CC3)cc2)cc1]

A sealed vial was charged with 1-bromo-4-cyclopropoxybenzene (97%, 300 mg, 1.37 mmol) and, DIPEA (477 μL, 2.73 mmol) in anhydrous dioxane (13.657 mL). The reaction mixture was degassed with argon for 5 min. Then, Xantphos (79 mg, 0.137 mmol), tris(dibenzylideneacetone)dipalladium(0) (63 mg, 0.0683 mmol), and benzyl mercaptan (321 μL, 2.73 mmol) were added and the reaction mixture was stirred at 105° C. overnight. The mixture was cooled to rt, filtered on a pad of dicalite, and washed with dioxane and DCM. A half saturated solution of Na₂S₂O₃ was added, and the aqueous layer was extracted twice with DCM. The organic layer was filtered through a phase separator, concentrated in vacuo, and purified by flash chromatography on silica gel using a gradient of EtOAc in heptane from 5% to 20%. Desired fractions were gathered, concentrated, dissolved in DCM, and washed three times with a solution of 5 mL saturated Na₂CO₃ in 45 mL water (pH 11-12) and concentrated in vacuo to afford the title product as an orange solid (367 mg, 97.5% yield, tᵣ=1.07 min). LCMS (Method F): m/z found 256.2 [M+H]⁺; ¹H-NMR (400 MHz, DMSO-d₆) δ (ppm) 7.35-7.22 (m, 7H), 7.01-6.96 (m, 2H), 4.11 (s, 2H), 3.81 (tt, J=6.0, 3.0 Hz, 1H), 0.81-0.73 (m, 2H), 0.67-0.59 (m, 2H).

### Step 2: Synthesis of 4-cyclopropoxy-N-(1-(3,4-dichlorophenyl)-2-(dimethylamino)ethyl)benzenesulfonamide (154)

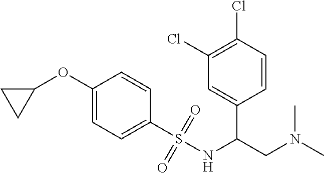 [SMILES: CN(C)CC(NS(=O)(=O)c1ccc(OC2CC2)cc1)c1ccc(Cl)c(Cl)c1]

In a round-bottomed flask, to a stirred solution of 1-benzylsulfanyl-4-(cyclopropoxy)benzene (367 mg, 1.43 mmol) in acetonitrile (13.474 mL) at 0° C. under air were added successively water (0.3365 mL), acetic acid (0.5049 mL) and then 1,3-dichloro-5,5-dimethyl-imidazolidine-2,4-dione (564 mg, 2.86 mmol). The reaction mixture was stirred at 0° C. for 2 h, then 1-(3,4-dichlorophenyl)-N²,N²-dimethylethane-1,2-diamine (95%, 439 mg, 1.79 mmol) and triethylamine (0.22 mL, 1.57 mmol) were added in sequence (the base was added dropwise), and the mixture was warmed to rt, and stirred overnight. The mixture was concentrated in vacuo and purified by flash chromatography on silica gel using a gradient of methanol in DCM from 0% to 10% a second time using an isocratic of 2% MeOH (0.7 N NH₃) in DCM, and one final time by reverse-phase chromatography using a gradient of acetonitrile in water from 0% to 100% (0.10% TFA). Desired fractions were gathered and concentrated, then saturated solution of NaHCO₃ was added (5 mL), and the aqueous layer was extracted three times using DCM. Organic layers were filtered through phase separator, and concentrated in vacuo. Diethyl ether and 2 M hydrogen chloride in diethyl ether (2.1 mL, 4.29 mmol) were added and the mixture was stirred 3 h at rt and concentrated in vacuo to afford the title compound as a white solid (82.3 mg, 12% yield, 99.8% purity, tᵣ=1.37 min). LCMS (Method C): m/z found 429 [M+H]⁺; ¹H-NMR (DMSO-d₆, 500 MHz): δ (ppm) 9.65 (br s, 1H), 8.58 (br d, J=10.0 Hz, 1H), 7.42-7.47 (m, 2H), 7.38-7.41 (m, 2H), 7.22 (dd, J=8.3, 2.0 Hz, 1H), 6.91 (d, J=8.8 Hz, 2H), 4.73-4.83 (m, 1H), 3.81 (tt, J=6.0, 2.9 Hz, 1H), 3.33-3.40 (m, 1H), 3.15-3.27 (m, 1H), 2.77-2.95 (m, 6H), 0.80 (dd, J=6.2, 1.6 Hz, 2H), 0.58-0.70 (m, 2H).

### Example 41: 2-amino-N-(1-(3,4-dichlorophenyl)-2-(dimethylamino)ethyl)-4-(trifluoromethoxy)benzenesulfonamide (155)

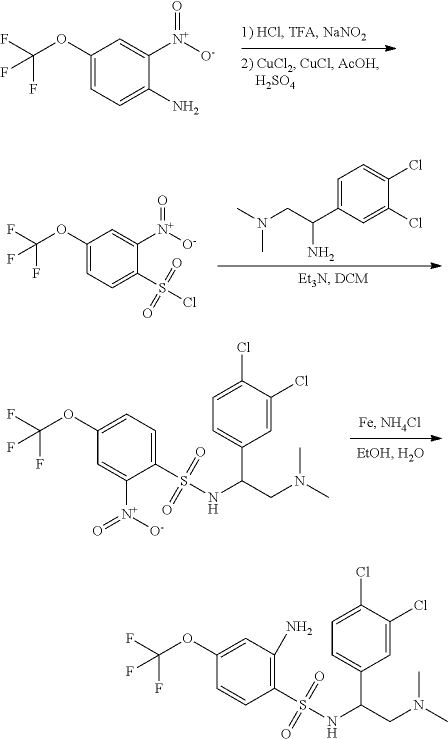 [SMILES: Nc1ccc(OC(F)(F)F)cc1[N+](=O)[O-] ; O=[N+]([O-])c1cc(OC(F)(F)F)ccc1S(=O)(=O)Cl ; CN(C)CC(N)c1ccc(Cl)c(Cl)c1 ; CN(C)CC(NS(=O)(=O)c1ccc(OC(F)(F)F)cc1[N+](=O)[O-])c1ccc(Cl)c(Cl)c1 ; CN(C)CC(NS(=O)(=O)c1ccc(OC(F)(F)F)cc1N)c1ccc(Cl)c(Cl)c1]

### Step 1: Synthesis of 2-nitro-4-(trifluoromethoxy)benzenesulfonyl chloride

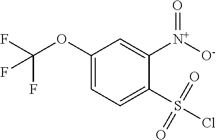 [SMILES: O=[N+]([O-])c1cc(OC(F)(F)F)ccc1S(=O)(=O)Cl]

In a three necked round bottomed flask equipped with a thermometer, a solution of 2-nitro-4-(trifluoromethoxy)aniline (98%, 200 mg, 0.882 mmol) in TFA (3 mL) and concentrated hydrogen chloride (295 μL, 9.71 mmol) was stirred at 0° C. Sodium nitrite (77 mg, 1.10 mmol) was added portion-wise over 15 min to keep the temperature below 5° C. The mixture was stirred at 0° C. for 30 min to afford Solution A. In a three necked round bottomed flask equipped with a thermometer, a solution of copper(II) dichloride (61 mg, 0.450 mmol) and copper(I) chloride (3.6 mg, 0.0265 mmol) in acetic acid (3 mL) and sulfuric acid (3.0 mL, 38.8 mmol) was stirred at 0° C. Solution A was added dropwise over 5 min to keep the temperature below 5° C. The mixture was stirred at 0° C. for 5 min then a rt for 1 h and was extracted twice with DCM. The combined organic layers were dried over sodium sulfate, filtered and concentrated to afford the title product as a red liquid (70 mg, 26% yield). ¹H-NMR (DMSO-d₆, 400 MHz) δ (ppm) 7.96 (d, J=8.7 Hz, 1H), 7.83 (d, J=2.0 Hz, 1H), 7.65-7.58 (m, 1H).

### Step 2: Synthesis of N-[1-(3,4-dichlorophenyl)-2-(dimethylamino)ethyl]-2-nitro-4-(trifluoromethoxy)benzenesulfonamide

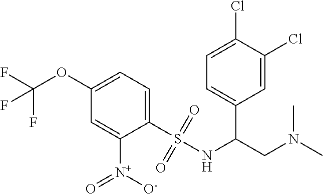 [SMILES: CN(C)CC(NS(=O)(=O)c1ccc(OC(F)(F)F)cc1[N+](=O)[O-])c1ccc(Cl)c(Cl)c1]

In a sealed tube, a solution of 2-nitro-4-(trifluoromethoxy)benzenesulfonyl chloride (65 μL, 0.229 mmol) in DCM (250 μL) was added to a stirred solution of 1-(3,4-dichlorophenyl)-N²,N²-dimethylethane-1,2-diamine (95%, 62 mg, 0.252 mmol) and triethylamine (128 μL, 0.916 mmol) in DCM (1.2 mL). The solution was stirred at rt for 20 h and was diluted with an half saturated aqueous solution of NaHCO₃. The aqueous layer was extracted with DCM, the combined organic layers were washed with a saturated aqueous solution of NaHCO₃, brine, dried over sodium sulfate, filtered and concentrated. The crude was purified by flash chromatography on silica gel using a gradient of (MeOH+2% NH₄OH) in DCM from 0.2% to 5%. The desired fractions were concentrated and pentane was added to the residue. A few drops of Et₂O were added and the obtained suspension was triturated and filtered. The residue was washed with pentane and was dried under reduced pressure at 40° C. for 16 h to afford the title product as a beige powder. (87 mg, 72% yield, tᵣ=0.71 min). LCMS (Method E): m/z found 502.2 [M+H]⁺; ¹H-NMR (500 MHz, DMSO-d₆, 300 K) δ (ppm) 9.31-8.20 (m, 1H), 8.12 (d, J=2.4 Hz, 1H), 8.06 (d, J=8.8 Hz, 1H), 7.73 (dd, J=1.2, 8.8 Hz, 1H), 7.51 (d, J=2.0 Hz, 1H), 7.46 (d, J=8.3 Hz, 1H), 7.26 (dd, J=2.0, 8.3 Hz, 1H), 4.45 (br dd, J=6.4, 8.1 Hz, 1H), 2.56 (br t, J=10.8 Hz, 1H), 2.31 (br dd, J=5.4, 12.5 Hz, 1H), 2.05 (s, 6H).

### Step 3: Synthesis of 2-amino-N-(1-(3,4-dichlorophenyl)-2-(dimethylamino)ethyl)-4-(trifluoromethoxy)benzenesulfonamide (155)

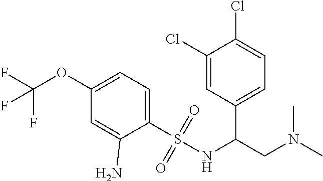 [SMILES: CN(C)CC(NS(=O)(=O)c1ccc(OC(F)(F)F)cc1N)c1ccc(Cl)c(Cl)c1]

In a sealed tube, a suspension of iron (34 mg, 0.602 mmol) and ammonium chloride (8.2 mg, 0.153 mmol) in ethanol (650 μL) and water (220 μL) was stirred at 70° C. for 1 h. N-[1-(3,4-dichlorophenyl)-2-(dimethylamino)ethyl]-2-nitro-4-(trifluoromethoxy)benzenesulfonamide (55 mg, 0.109 mmol) was added and the mixture was stirred at 80° C. for 1 h. The mixture was allowed to cool to rt and was filtered through a pad of dicalite. The pad was washed with EtOH and the filtrate was concentrated. The crude was purified by flash chromatography on silica gel using a gradient of (MeOH+2% NH₄OH) in DCM from 1% to 10%. The desired fractions were concentrated and pentane was added to the residue. The obtained suspension was triturated and filtered. The residue was washed with pentane and dried under reduced pressure at 40° C. for 16 h to afford the title compound as a white powder (38 mg, 73% yield, 100% purity, tᵣ=1.51 min). LCMS (Method C): m/z found 472 [M+H]⁺; ¹H-NMR (500 MHz, DMSO-d₆, 300 K) δ (ppm) 8.13 (br s, 1H), 7.44-7.33 (m, 3H), 7.15 (dd, J=2.2, 8.3 Hz, 1H), 6.56 (d, J=1.2 Hz, 1H), 6.38-6.30 (m, 1H), 6.18 (s, 2H), 4.25 (t, J=7.5 Hz, 1H), 2.42-2.34 (m, 1H), 2.33-2.26 (m, 1H), 2.07 (s, 6H).

### Example 42: N-(1-(3,4-dichlorophenyl)-2-(dimethylamino)ethyl)-2,2-difluorobenzo[d][1,3]dioxole-5-sulfonamide (156)

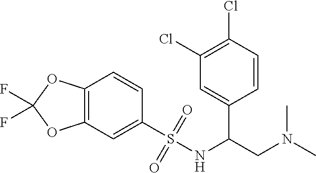 [SMILES: CN(C)CC(NS(=O)(=O)c1ccc2c(c1)OC(F)(F)O2)c1ccc(Cl)c(Cl)c1]

In a sealed vial under nitrogen, 2,2-difluoro-1,3-benzodioxole-5-sulfonyl chloride (95%, 60 mg, 0.222 mmol) was added to a stirred solution of 1-(3,4-dichlorophenyl)-N²,N²-dimethylethane-1,2-diamine (52 mg, 0.222 mmol) and triethylamine (0.12 mL, 0.889 mmol) in anhydrous DCM (2.2213 mL). The solution was stirred at rt for 20 h, then DCM and an half saturated solution of NaHCO₃ were added, the aqueous layer was extracted with DCM, then the combined organic layers were concentrated in vacuo and purified by flash chromatography on silica gel using a gradient of MeOH in DCM from 0% to 6%. Desired fraction were gathered and concentrated in vacuo, 2 M hydrogen chloride in diethyl ether (0.11 mL, 0.222 mmol) was added, the mixture was stirred overnight and concentrated in vacuo to afford the title compound as a white powder (64.2 mg, 58% yield, 99.3% purity, tᵣ=1.46 min). LCMS (Method C): m/z found 453 [M+H]⁺; ¹H-NMR (500 MHz, DMSO-d₆, 300 K) δ (ppm) 9.71 (br s, 1H), 8.93 (br d, J=9.8 Hz, 1H), 7.47-7.37 (m, 5H), 7.26 (dd, J=2.2, 8.3 Hz, 1H), 5.03-4.63 (m, 1H), 3.55-3.36 (m, 1H), 3.24 (br d, J=8.6 Hz, 1H), 2.97-2.72 (m, 6H).

### Example 43: N-(1-(3,4-dichlorophenyl)-2-(dimethylamino)ethyl)-6-(trifluoromethoxy)pyridine-3-sulfonamide (157)

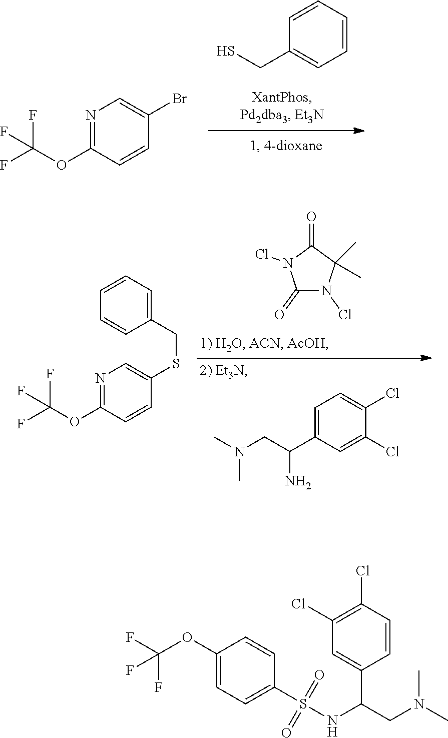 [SMILES: SCc1ccccc1 ; FC(F)(F)Oc1ccc(Br)cn1 ; FC(F)(F)Oc1ccc(SCc2ccccc2)cn1 ; CN(C)CC(N)c1ccc(Cl)c(Cl)c1 ; CC1(C)C(=O)N(Cl)C(=O)N1Cl ; CN(C)CC(NS(=O)(=O)c1ccc(OC(F)(F)F)cc1)c1ccc(Cl)c(Cl)c1]

### Step 1: Synthesis of 5-benzylsulfanyl-2-(trifluoromethoxy)pyridine

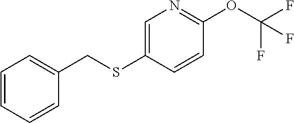 [SMILES: FC(F)(F)Oc1ccc(SCc2ccccc2)cn1]

A sealed vial was charged with 5-bromo-2-(trifluoromethoxy)pyridine (95%, 300 mg, 1.18 mmol) and, DIPEA (411 μL, 2.36 mmol) in anhydrous 1,4-dioxane (11.777 mL). The reaction mixture was degassed with argon for 5 min. Then, Xantphos (68 mg, 0.118 mmol), tris(dibenzylideneacetone)dipalladium(0) (54 mg, 0.0589 mmol) and phenylmethanethiol (277 μL, 2.36 mmol) were added, and the reaction mixture was stirred at 105° C. overnight. The mixture was cooled to rt, filtered on a pad of dicalite, washed with dioxane and DCM, concentrated and purified by flash chromatography on silica gel using a gradient of EtOAc in heptane from 0 to 10% to afford the title compound as a colorless or orange oil (254.2 mg, 73% yield, tᵣ=1.02 min). LCMS (Method F): m/z found 286.2 [M+H]⁺; ¹H-NMR (400 MHz, DMSO-d₆) δ (ppm) 8.28 (d, J=2.3 Hz, 1H), 7.99 (dd, J=8.6, 2.6 Hz, 1H), 7.34-7.22 (m, 6H), 4.31 (s, 2H).

### Step 2: Synthesis of N-(1-(3,4-dichlorophenyl)-2-(dimethylamino)ethyl)-6-(trifluoromethoxy)pyridine-3-sulfonamide (157)

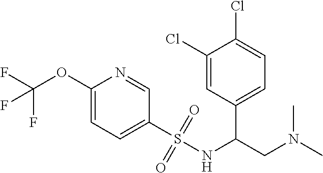 [SMILES: CN(C)CC(NS(=O)(=O)c1ccc(OC(F)(F)F)nc1)c1ccc(Cl)c(Cl)c1]

In a round-bottomed flask, to a stirred solution of 5-benzylsulfanyl-2-(trifluoromethoxy)pyridine (207 mg, 0.726 mmol) in acetonitrile (6.8296 mL) at 0° C. under air were added successively water (0.1705 mL), acetic acid (0.2559 mL) and then 1,3-dichloro-5,5-dimethyl-imidazolidine-2,4-dione (286 mg, 1.45 mmol). The reaction mixture was stirred at 0° C. for 2 h, then 1-(3,4-dichlorophenyl)-N²,N²-dimethylethane-1,2-diamine (95%, 196 mg, 0.798 mmol) and triethylamine (0.11 mL, 0.798 mmol) were added in sequence (the base was added dropwise), and the mixture was warmed to rt, and stirred overnight. A half saturated solution of NH₄Cl was added, and the mixture was extracted twice with DCM. Combined organic layers were washed with an half saturated solution of NaHCO₃, then dried over Na₂SO₄, concentrated in vacuo and purified by flash chromatography on silica gel using a gradient of Methanol (0.7 N NH₃) in DCM from 0.4% to 10%. Desired fractions were combined, concentrated in vacuo then taken in diethyl ether and 2 M hydrogen chloride in diethyl ether (1.1 mL, 2.18 mmol) was added and the mixture was stirred 3 hours at rt and concentrated in vacuo to afford the title compound as a white powder (12.2 mg, 3% yield, 98.2% purity, tᵣ=1.38 min). LCMS (Method C): m/z found 458 [M+H]⁺; ¹H-NMR (500 MHz, DMSO-d₆) δ (ppm) 9.44-9.71 (m, 1H), 9.11 (br d, J=9.5 Hz, 1H), 8.42 (d, J=2.4 Hz, 1H), 8.09 (dd, J=8.7, 2.5 Hz, 1H), 7.48 (d, J=1.7 Hz, 1H), 7.42 (d, J=8.3 Hz, 1H), 7.25 (d, J=8.6 Hz, 2H), 4.74-5.04 (m, 1H), 3.39-3.53 (m, 2H), 2.77-2.98 (m, 6H).

### Example 44: N-(1-(3,4-dichlorophenyl)-2-(dimethylamino)ethyl)-4-ethoxybenzenesulfonamide (158)

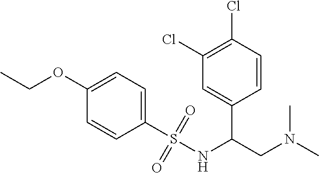 [SMILES: CCOc1ccc(S(=O)(=O)NC(CN(C)C)c2ccc(Cl)c(Cl)c2)cc1]

In a sealed vial under nitrogen, 4-ethoxybenzenesulfonyl chloride (95%, 50 mg, 0.214 mmol) was added to a stirred solution of 1-(3,4-dichlorophenyl)-N²,N²-dimethylethane-1,2-diamine (50 mg, 0.214 mmol) and triethylamine (0.12 mL, 0.858 mmol) in anhydrous DCM (2.1446 mL). The solution was stirred at rt overnight, concentrated in vacuo and purified by flash chromatography on silica gel using a gradient of MeOH in DCM from 0% to 4%. Desired fractions were gathered, concentrated in vacuo, dissolved in Et₂O, precipitated using pentane and filtered to afford the title compound as a white powder (50.8 mg, 57% yield, 99.6% purity, tᵣ=1.52 min). LCMS (Method C): m/z found 417 [M+H]⁺; ¹H-NMR (600 MHz, DMSO-d₆, 300 K) δ (ppm) 7.89 (br d, J=0.6 Hz, 1H), 7.48 (d, J=8.8 Hz, 2H), 7.39 (d, J=8.2 Hz, 1H), 7.31 (d, J=2.1 Hz, 1H), 7.14 (dd, J=2.1, 8.2 Hz, 1H), 6.87 (d, J=8.8 Hz, 2H), 4.40-4.18 (m, 1H), 4.05 (dq, J=1.8, 7.0 Hz, 2H), 2.40-2.21 (m, 2H), 2.04 (br s, 6H), 1.32 (t, J=7.0 Hz, 3H).

### Example 45: 3,4-dichloro-N-(1-(3,4-dichlorophenyl)-2-(dimethylamino)ethyl)benzenesulfonamide (159)

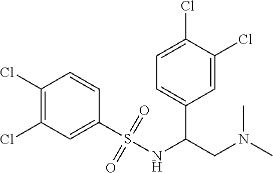 [SMILES: CN(C)CC(NS(=O)(=O)c1ccc(Cl)c(Cl)c1)c1ccc(Cl)c(Cl)c1]

In a sealed tube, a solution of 3,4-dichlorobenzenesulfonyl chloride (39 μL, 0.244 mmol) in DCM (500 μL) was added to a stirred solution of 1-(3,4-dichlorophenyl)-N²,N²-dimethylethane-1,2-diamine (63 mg, 0.269 mmol) and triethylamine (136 μL, 0.978 mmol) in DCM (1 mL). The solution was stirred at rt for 18 h and was diluted with half saturated aqueous NaHCO₃. The aqueous layer was extracted with DCM, the combined organic layers were washed with brine, dried over sodium sulfate, filtered and concentrated. The crude was purified by flash chromatography on silica gel using a gradient of (MeOH+2% NH₄OH) in DCM from 0.2% to 10%. The residue was triturated with pentane and the suspension was filtered. The residue was washed with pentane and dried under reduced pressure at 45° C. for 16 h to afford the title compound as a white powder (56 mg, 51% yield, 99.8% purity, tᵣ=1.68 min). LCMS (Method C): m/z found 441 [M+H]⁺; ¹H-NMR (DMSO-d₆, 500 MHz): δ (ppm) 8.41 (br s, 1H), 7.67 (d, J=8.3 Hz, 1H), 7.62 (d, J=2.0 Hz, 1H), 7.51 (dd, J=8.4, 2.1 Hz, 1H), 7.41 (d, J=8.3 Hz, 1H), 7.30 (d, J=2.0 Hz, 1H), 7.16 (dd, J=8.3, 2.2 Hz, 1H), 4.40 (t, J=7.5 Hz, 1H), 2.45-2.49 (m, 1H), 2.30 (dd, J=12.5, 6.6 Hz, 1H), 2.09 (s, 6H).

### Example 46: 4-chloro-N-(1-(3,4-dichlorophenyl)-2-(dimethylamino)ethyl)-2-fluorobenzenesulfonamide (160)

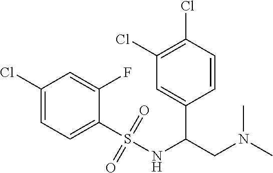 [SMILES: CN(C)CC(NS(=O)(=O)c1ccc(Cl)cc1F)c1ccc(Cl)c(Cl)c1]

In a sealed tube, 4-chloro-2-fluorobenzenesulfonyl chloride (47 mg, 0.204 mmol) was added to a stirred solution of 1-(3,4-dichlorophenyl)-N′,N′-dimethyl-ethane-1,2-diamine (95%, 50 mg, 0.204 mmol) and triethylamine (85 μL, 0.611 mmol) in anhydrous DCM (2.2 mL). The solution was stirred at room temperature for 20 h. Water (5 mL) was added and the aqueous layer was extracted with ethyl acetate (1×10 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was triturated in pentane, filtered, washed with pentane and dried under vacuum at 60° C. for 18 h to afford the title compound as a white powder (66.2 mg, 76% yield, 99.8% purity, tᵣ=1.58 min). LCMS (Method C): m/z found 424.9 [M+H]⁺; ¹H-NMR (500 MHz, DMSO-d₆) δ (ppm) 8.22-9.03 (m, 1H) 7.65 (t, J=8.19 Hz, 1H) 7.47 (dd, J=10.27, 1.96 Hz, 1H) 7.44 (d, J=8.31 Hz, 1H) 7.39-7.41 (m, 1H) 7.29-7.34 (m, 1H) 7.17-7.23 (m, 1H) 4.21-4.43 (m, 1H) 2.51-2.54 (m, 1H) 2.26-2.35 (m, 1H) 2.03 (s, 6H).

### Example 47: N-(1-(3,4-dichlorophenyl)-2-(dimethylamino)ethyl)-4-(trifluoromethyl)benzenesulfonamide (161)

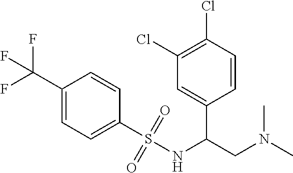 [SMILES: CN(C)CC(NS(=O)(=O)c1ccc(C(F)(F)F)cc1)c1ccc(Cl)c(Cl)c1]

In a sealed vial under nitrogen, 4-(trifluoromethyl)benzenesulfonyl chloride (98%, 54 mg, 0.214 mmol) was added to a stirred solution of 1-(3,4-dichlorophenyl)-N²,N²-dimethylethane-1,2-diamine (50 mg, 0.214 mmol) and triethylamine (0.12 mL, 0.858 mmol) in anhydrous DCM (2.1446 mL). The solution was stirred at rt overnight, then DCM and an half saturated solution of NaHCO₃ were added, the aqueous layer was extracted twice with DCM, the combined organic layers were dried through phase separator, concentrated in vacuo and purified by flash chromatography on silica gel using a gradient of MeOH in DCM from 0% to 2% to afford the title compound as a white powder (28.2 mg, 30% yield, 99.7% purity, tᵣ=1.69 min). LCMS (Method C): m/z found 440.9 [M+H]⁺; ¹H-NMR (500 MHz, DMSO-d₆) δ (ppm) 8.11-8.76 (m, 1H) 7.70-7.79 (m, 4H) 7.31-7.41 (m, 1H) 7.24-7.29 (m, 1H) 7.12-7.18 (m, 1H) 4.30-4.49 (m, 1H) 2.26-2.48 (m, 2H) 2.06 (s, 6H).

### Example 48: N-(1-(3,4-dichlorophenyl)-2-(dimethylamino)ethyl)-3-(trifluoromethyl)benzenesulfonamide (162)

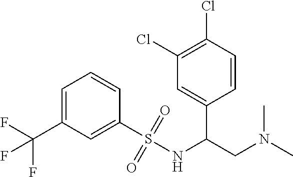 [SMILES: CN(C)CC(NS(=O)(=O)c1cccc(C(F)(F)F)c1)c1ccc(Cl)c(Cl)c1]

In a sealed vial under nitrogen, 3-(trifluoromethyl)benzenesulfonyl chloride (0.034 mL, 0.214 mmol) was added to a stirred solution of 1-(3,4-dichlorophenyl)-N²,N²-dimethylethane-1,2-diamine (50 mg, 0.214 mmol) and triethylamine (0.12 mL, 0.858 mmol) in anhydrous DCM (2.1446 mL). The solution was stirred at rt for 20 h, concentrated in vacuo and purified by flash chromatography on silica gel using a gradient of MeOH in DCM from 0% to 10%. Desired fraction were gathered, concentrated in vacuo, 2 M hydrogen chloride in diethyl ether (2.1 mL, 4.29 mmol) was added and the mixture was stirred overnight at rt and concentrated in vacuo to afford the title compound as a white powder (31.2 mg, 30% yield, 98.3% purity, tᵣ=4.75 min). LCMS (Method G): m/z found 440.9 [M+H]⁺; ¹H-NMR (500 MHz, DMSO-d₆) δ (ppm) 9.37-9.82 (m, 1H) 8.84-9.15 (m, 1H) 7.88 (d, J=7.82 Hz, 1H) 7.83 (d, J=8.07 Hz, 1H) 7.71 (s, 1H) 7.64 (t, J=7.80 Hz, 1H) 7.42 (s, 1H) 7.33 (d, J=8.31 Hz, 1H) 7.19 (br dd, J=8.31, 1.70 Hz, 1H) 4.78-4.98 (m, 1H) 3.37-3.53 (m, 1H) 3.15-3.28 (m, 1H) 2.69-3.04 (m, 6H).

### Example 49: 3-chloro-N-(1-(3,4-dichlorophenyl)-2-(dimethylamino)ethyl)benzenesulfonamide (163)

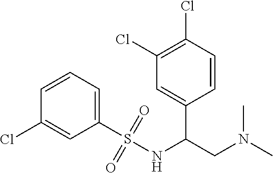 [SMILES: CN(C)CC(NS(=O)(=O)c1cccc(Cl)c1)c1ccc(Cl)c(Cl)c1]

In a sealed tube, 3-chlorobenzenesulfonyl chloride (98%, 25 μL, 0.175 mmol) was added to a stirred solution of 1-(3,4-dichlorophenyl)-N′,N′-dimethyl-ethane-1,2-diamine (95%, 43 mg, 0.175 mmol) and triethylamine (73 μL, 0.526 mmol) in anhydrous DCM (2 mL). The solution was stirred at room temperature for 20 h. Water (5 mL) was added and the aqueous layer was extracted with ethyl acetate (1×10 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 4%. The residue was triturated in pentane, filtered, washed with pentane and dried under vacuum at 60° C. for 18 h to afford the title compound as a white powder (52.4 mg, 74% yield, 100% purity, tᵣ=1.58 min). LCMS (Method C): m/z found 406.9 [M+H]⁺; ¹H-NMR (500 MHz, DMSO-d₆) δ (ppm) 8.34 (br s, 1H), 7.42-7.58 (m, 4H), 7.39-7.42 (m, 1H), 7.35-7.37 (m, 1H), 7.16 (dd, J=1.96, 8.31 Hz, 1H), 4.39 (t, J=7.58 Hz, 1H), 2.43 (dd, J=8.44, 12.59 Hz, 1H), 2.30 (dd, J=6.72, 12.59 Hz, 1H), 2.07 (s, 6H).

### Example 50: 4-chloro-N-(1-(3,4-dichlorophenyl)-2-(dimethylamino)ethyl)benzenesulfonamide (164)

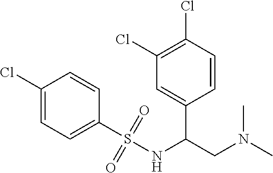 [SMILES: CN(C)CC(NS(=O)(=O)c1ccc(Cl)cc1)c1ccc(Cl)c(Cl)c1]

In a sealed tube, 4-chlorobenzenesulfonyl chloride (97%, 46 mg, 0.212 mmol) was added to a stirred solution of 1-(3,4-dichlorophenyl)-N′,N′-dimethyl-ethane-1,2-diamine (99%, 50 mg, 0.212 mmol) and triethylamine (89 μL, 0.637 mmol) in anhydrous DCM (2.2 mL). The solution was stirred at room temperature for 20 h. Water (5 mL) was added and the aqueous layer was extracted with ethyl acetate (1×10 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 4%. The residue was triturated in pentane, filtered, washed with pentane and dried under vacuum at 70° C. for 18 h to afford the title compound as a white powder (57.7 mg, 67% yield, 99.8% purity, tᵣ=1.59 min). LCMS (Method C): m/z found 406.9 [M+H]⁺; ¹H-NMR (600 MHz, DMSO-d₆) δ (ppm) 8.24 (br s, 1H), 7.57 (d, J=8.12 Hz, 2H), 7.43-7.48 (m, 2H), 7.37-7.42 (m, 1H), 7.31 (d, J=1.91 Hz, 1H), 7.16 (dd, J=2.05, 8.22 Hz, 1H), 4.33 (t, J=7.56 Hz, 1H), 2.40 (dd, J=8.22, 12.62 Hz, 1H), 2.30 (dd, J=6.97, 12.54 Hz, 1H), 2.05 (s, 6H).

### Example 51: N-(1-(3,4-dichlorophenyl)-2-(dimethylamino)ethyl)-4-(trifluoromethoxy)benzenesulfonimidamide (165a and 165b

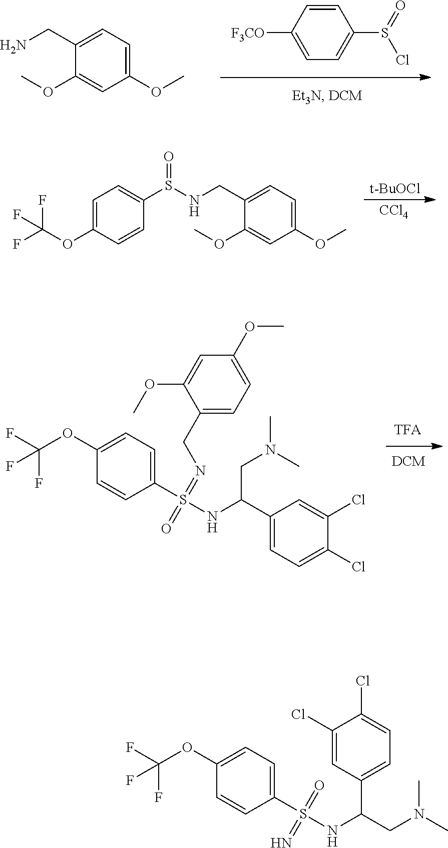

### Step 1: Synthesis of N-[(2,4-dimethoxyphenyl)methyl]-4-(trifluoromethoxy)benzenesulfinamide

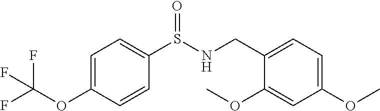 [SMILES: COc1ccc(CNS(=O)c2ccc(OC(F)(F)F)cc2)c(OC)c1]

In a round-bottomed flask, to a stirred suspension of 4-(trifluoromethoxy)benzenesulfinyl chloride (73%, 3.36 g, 10.0 mmol) in anhydrous DCM (25 mL) at room temperature under nitrogen were added successively 1-(2,4-dimethoxyphenyl)methanamine (2.3 mL, 15.0 mmol) and triethylamine (4.2 mL, 30.1 mmol). The reaction mixture was stirred at room temperature for 20 h. Water (100 mL) was added and the aqueous layer was extracted with dichloromethane (1×20 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5% to afford the expected compound as a yellow powder (1.803 g, 43% yield, tᵣ=0.91). LCMS (Method F): m/z found 376.2 [M+H]⁺; ¹H-NMR (DMSO-d₆, 400 MHz) δ (ppm) 7.79-7.69 (m, 2H), 7.54 (d, J=8.1 Hz, 2H), 7.03 (d, J=8.3 Hz, 1H), 6.90 (dd, J=7.3, 4.8 Hz, 1H), 6.48 (d, J=2.4 Hz, 1H), 6.44 (dd, J=8.3, 2.4 Hz, 1H), 3.90 (dd, J=13.9, 4.7 Hz, 1H), 3.73 (s, 3H), 3.70 (s, 3H), 3.62 (dd, J=14.0, 7.4 Hz, 1H).

### Step 2: Synthesis of 1-(3,4-dichlorophenyl)-N—[N-[(2,4-dimethoxyphenyl)methyl]-S-[4-(trifluoromethoxy)phenyl]sulfonimidoyl]-N′,N′-dimethyl-ethane-1,2-diamine

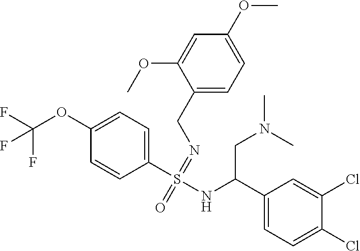 [SMILES: COc1ccc(CN=S(=O)(NC(CN(C)C)c2ccc(Cl)c(Cl)c2)c2ccc(OC(F)(F)F)cc2)c(OC)c1]

In a round bottomed flask 1.37 M sodium hypochlorite (154 mL, 0.211 mol) was stirred at 0° C. in the dark. A mixture of tert-butanol (10 mL, 0.105 mol) and acetic acid (6.6 mL, 0.116 mol) was added and the mixture was stirred at 0° C. in the dark for 10 min. The organic layer was washed with a 10% aqueous solution of NaHCO₃, with water and was dried over calcium chloride to afford t-butyl hypochlorite as a yellow liquid (9 g, 60% yield). The product was used in the subsequent reaction without further purification.

In a sealed tube under nitrogen, in the dark, a solution of N-[(2,4-dimethoxyphenyl)methyl]-4-(trifluoromethoxy)benzenesulfinamide (400 mg, 1.07 mmol) and tert-butyl hypochlorite (121 mg, 1.12 mmol) in Carbon tetrachloride (8 mL) was stirred at 0° C. for 1 h. The mixture was concentrated under reduced pressure at 2° C. The residue was diluted in dry THF (8 mL) under nitrogen and the solution was stirred at 0° C. 1-(3,4-dichlorophenyl)-N²,N²-dimethylethane-1,2-diamine (95%, 275 mg, 1.12 mmol) and N,N-diisopropylethylamine (558 μL, 3.20 mmol) were added at 0° C. then the mixture was stirred at rt for 4 days. The mixture was diluted with an half saturated aqueous solution of NaHCO₃ and EtOAc. The aqueous layer was extracted twice with EtOAc, the combined organic layers were washed with brine, dried over sodium sulfate, filtered and concentrated. The crude was purified by flash chromatography on silica gel using a gradient of (MeOH+2% NH₄OH) from 0.2% to 5% to afford a mixture of all four stereoisomers of the title compound (340 mg, 51% yield, tᵣ=0.80 min). LCMS (Method F): m/z found 606.3 [M+H]⁺; ¹H-NMR (DMSO-d₆, 400 MHz): δ (ppm) 7.93 (dd, J=8.9, 2.3 Hz, 2H), 7.53-6.93 (m, 8H), 6.68-6.32 (m, 2H), 4.39 (m, 1H), 4.02-3.56 (m, 8H), 2.42 (d, J=6.9 Hz, 2H), 2.03 (d, J=16.6 Hz, 7H).

### Step 3: Synthesis of N-(1-(3,4-dichlorophenyl)-2-(dimethylamino)ethyl)-4-(trifluoromethoxy)benzenesulfonimidamide (165a and 165b

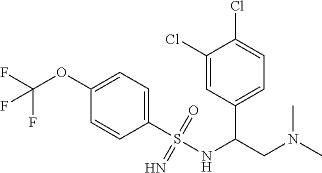 [SMILES: CN(C)CC(NS(=N)(=O)c1ccc(OC(F)(F)F)cc1)c1ccc(Cl)c(Cl)c1]

In a sealed tube under nitrogen, 2,2,2-trifluoroacetic acid (1.874 mL, 25.2 mmol) was added to a stirred solution of 1-(3,4-dichlorophenyl)-N—[N-[(2,4-dimethoxyphenyl)methyl]-S-[4-(trifluoromethoxy)phenyl]sulfonimidoyl]-N′,N′-dimethyl-ethane-1,2-diamine (180 mg, 0.297 mmol) in DCM (1.9 mL). The purple mixture was stirred at rt for 16 h and was poured into an half saturated solution of NaHCO₃. The mixture was extracted twice with DCM, the combined organic layers were washed with brine, dried over sodium sulfate, filtered and concentrated. The crude was purified by flash chromatography on silica gel using a gradient of (MeOH+2% NH₄OH) in DCM from 1% to 10%. The desired fractions were concentrated and the residue was purified by reverse phase chromatography using a gradient of (ACN+0.1% AcOH) in (H2O+0.1% AcOH) from 10% to 100%. The desired fractions were concentrated and the residue was dissolved in DCM. The organic layer was washed twice with a saturated aqueous solution of NaHCO₃, with brine, dried over sodium sulfate, filtered and concentrated. Pentane was added to the residue, followed by a few drops of Et₂O and the obtained suspension was triturated and filtered. The residue was washed with pentane and dried under reduced pressure at 40° C. for 16 h to afford the title compound (165a, racemic mixture) as a white powder (47 mg, 34% yield, 99.4% purity, tᵣ=1.46 min). LCMS (Method C): m/z found 456 [M+H]⁺; ¹H-NMR (600 MHz, DMSO-d₆, 300 K) δ (ppm) 7.82 (br d, J=8.5 Hz, 2H), 7.57-6.85 (m, 6H), 5.44-4.45 (m, 1H), 4.29 (t, J=7.4 Hz, 1H), 2.36-2.24 (m, 2H), 2.00 (s, 6H).

The diastereomers of 165a (i.e. 165b) may be obtained following an identical experimental procedure with alternative purification methods as described herein.

Similarly, in a sealed tube under nitrogen, 2,2,2-trifluoroacetic acid (1.7 mL, 22.4 mmol) was added to a stirred solution of 1-(3,4-dichlorophenyl)-N—[N-[(2,4-dimethoxyphenyl)methyl]-S-[4-(trifluoromethoxy)phenyl]sulfonimidoyl]-N′,N′-dimethyl-ethane-1,2-diamine (160 mg, 0.264 mmol) in DCM (1.7 mL). The purple mixture was stirred at rt for 16 h and was poured into an half saturated solution of NaHCO₃. The mixture was extracted twice with DCM, the combined organic layers were washed with brine, dried over sodium sulfate, filtered and concentrated. The crude was purified by flash chromatography on silica gel using a gradient of (MeOH+2% NH₄OH) in DCM from 0.2% to 6%. The desired fractions were concentrated and pentane was added to the residue. The obtained suspension was triturated and filtered. The residue was washed with pentane and dried under reduced pressure at 40° C. for 16 h to afford the expected product (165b, racemic mixture) as a white powder (60 mg, 96.5% purity, 48% yield, tᵣ=1.49 min). LCMS (Method C): m/z found 455.9 [M+H]⁺; ¹H-NMR (600 MHz, CD₃SOCD₃, 300 K) δ (ppm)=7.74 (br d, J=7.3 Hz, 2H), 7.61-7.20 (m, 5H), 7.12 (br d, J=7.9 Hz, 1H), 5.28-3.87 (m, 2H), 2.42-2.35 (m, 1H), 2.35-2.27 (m, J=6.9 Hz, 1H), 2.06 (s, 6H).

### Example 52: N-(1-(3-chlorophenyl)-2-(dimethylamino)ethyl)-4-(trifluoromethoxy)benzenesulfonamide (166)

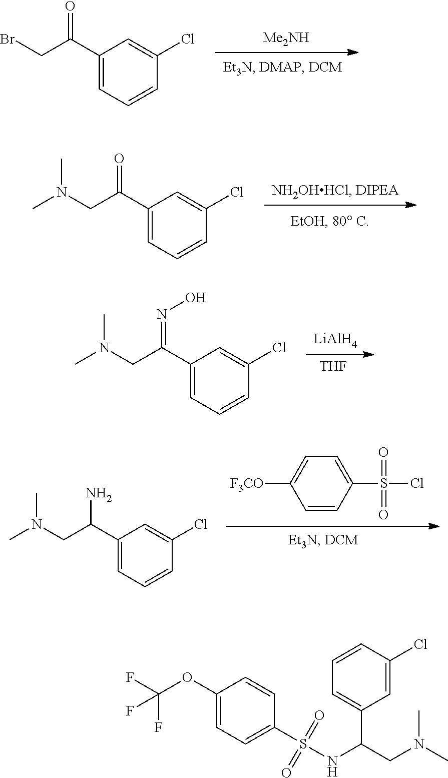

### Step 1: Synthesis of 1-(3-chlorophenyl)-2-(dimethylamino)ethanone

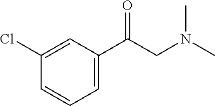

In a sealed tube under nitrogen, a solution of 2-bromo-1-(3-chlorophenyl)ethanone (1.00 g, 4.28 mmol) and DMAP (26 mg, 0.214 mmol) in dry DCM (10 mL) was stirred at rt 2 M dimethylamine (3.9 mL, 7.71 mmol) then triethylamine (657 μL, 4.71 mmol) were added and the solution was stirred at rt for 16 h. The mixture was washed with a half saturated aqueous solution of NaHCO₃. The aqueous layer was extracted twice with DCM. The combined organic layers were washed with brine, dried over sodium sulfate, filtered and concentrated. The crude was purified by flash chromatography on silica gel using a gradient of (MeOH+2% NH₄OH) in DCM from 0.4% to 2% to afford the title compound as a red oil (480 mg, 56% yield, tᵣ=0.56 min). LCMS (Method F): m/z found 198.2 [M+H]⁺; ¹H-NMR (DMSO-d₆, 400 MHz) δ (ppm) 7.98 (t, J=1.8 Hz, 1H), 7.93 (dt, J=7.8, 1.3 Hz, 1H), 7.71 (ddd, J=8.0, 2.2, 1.1 Hz, 1H), 7.56 (t, J=7.9 Hz, 1H), 3.76 (s, 2H), 2.25 (s, 6H).

### Step 2: Synthesis of 1-(3-chlorophenyl)-2-(dimethylamino)ethanone oxime

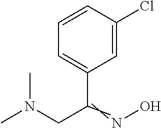

In a sealed tube, DIPEA (891 μL, 5.10 mmol) was added to a solution of hydroxylamine hydrochloride (338 mg, 4.86 mmol) and 1-(3-chlorophenyl)-2-(dimethylamino)ethanone (480 mg, 2.43 mmol) in ethanol (10 mL). The solution was stirred at 80° C. for 20 h and was concentrated under reduced pressure to afford the title compound as a yellow oil (1.38 g, quantitative yield, tᵣ=0.61 min). LCMS (Method F): m/z found 213.2 [M+H]⁺; ¹H-NMR (DMSO-d₆, 400 MHz) δ (ppm) 7.77-7.51 (m, 2H), 7.48-7.38 (m, 2H), 4.35 (t, J=4.8 Hz, 2H), 2.20 (d, J=4.7 Hz, 6H).

### Step 3: Synthesis of 1-(3-chlorophenyl)-N²,N²-dimethyl-ethane-1,2-diamine

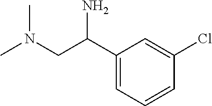

In a three necked round bottomed flask equipped with a thermometer and a condenser, under nitrogen, a solution of 1-(3-chlorophenyl)-2-(dimethylamino)ethanone oxime (516 mg, 2.43 mmol) in dry THF (10 mL) was stirred at 0° C. 2.4 M lithium aluminium hydride (2.5 mL, 6.07 mmol) was added dropwise over 2 min to keep the temperature below 10° C. The mixture was refluxed for 2 h, was allowed to cool to rt then was stirred at 0° C. 230 μL H₂O were added dropwise then 230 μL aqueous 15% NaOH were added dropwise. The mixture was stirred at 0° C. for 15 min then 700 μL H₂O and EtOAc were added. The mixture was stirred at rt for 15 min and filtered through a pad of decalite, the pad was washed with EtOAc and the filtrate was concentrated. The crude was purified by flash chromatography on silica gel using a gradient of (MeOH+2% NH₄OH) in DCM from 1% to 10% to afford the title compound as a yellow oil (260 mg, 53% yield, tᵣ=0.25 min). LCMS (Method F): m/z found 199.3 [M+H]⁺; ¹H-NMR (DMSO-d₆, 400 MHz) δ (ppm) 7.47-7.42 (m, 1H), 7.34-7.29 (m, 2H), 7.29-7.22 (m, 1H), 3.98 (dd, J=9.2, 5.0 Hz, 1H), 2.28 (dd, J=11.9, 9.2 Hz, 1H), 2.22-2.09 (m, 8H), 1.91 (bs, 2H).

### Step 4: Synthesis of N-(1-(3-chlorophenyl)-2-(dimethylamino)ethyl)-4-(trifluoromethoxy)benzenesulfonamide (166)

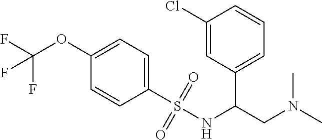 [SMILES: CN(C)CC(NS(=O)(=O)c1ccc(OC(F)(F)F)cc1)c1cccc(Cl)c1]

In a sealed tube, a solution of 4-(trifluoromethoxy)benzenesulfonyl chloride (39 μL, 0.230 mmol) in DCM (500 μL) was added to a stirred solution of 1-(3-chlorophenyl)-N²,N²-dimethyl-ethane-1,2-diamine (50 mg, 0.253 mmol) and triethylamine (128 μL, 0.921 mmol) in DCM (1 mL). The solution was stirred at rt for 18 h and was diluted with half saturated aqueous NaHCO₃. The aqueous layer was extracted with DCM, the combined organic layers were washed with brine, dried over sodium sulfate, filtered and concentrated. The crude was purified by flash chromatography on silica gel using a gradient of (MeOH+2% NH₄OH) in DCM from 0.2% to 10%. The desired fractions were concentrated and dissolved in Et₂O. The solution was added dropwise to stirred 2 N HCl/Et₂O and the obtained suspension was stirred at rt for 2 h. The suspension was filtered, the residue was washed with Et₂O and dried under reduced pressure at 45° C. for 16 h to afford the title compound as a white powder (56 mg, 52% yield, 99.4% purity, tᵣ=1.56 min). LCMS (Method C): m/z found 423 [M+H]⁺; ¹H-NMR (DMSO-d₆, 600 MHz): δ (ppm) 9.66 (br s, 1H), 8.89 (br d, J=9.5 Hz, 1H), 7.61-7.66 (m, 2H), 7.27 (d, J=8.2 Hz, 2H), 7.23-7.25 (m, 1H), 7.15-7.18 (m, 1H), 7.08-7.15 (m, 2H), 4.83 (t, J=9.4 Hz, 1H), 3.35-3.43 (m, 1H), 3.17-3.29 (m, 1H), 2.76-2.96 (m, 6H).

### Example 53: N-(1-(3-chloro-4-fluorophenyl)-2-(dimethylamino)ethyl)-4-(trifluoromethoxy)benzenesulfonamide (167)

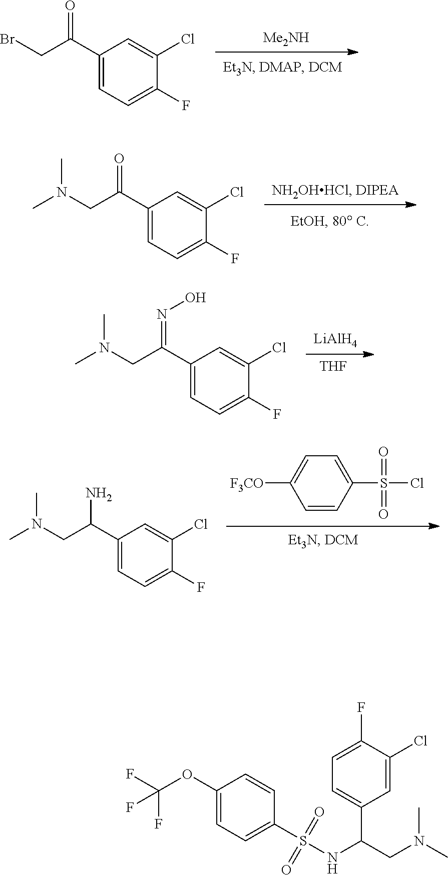

### Step 1: Synthesis of 1-(3-chloro-4-fluoro-phenyl)-2-(dimethylamino)ethanone

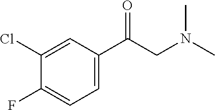

In a round-bottomed flask, to a stirred solution of 2-bromo-1-(3-chloro-4-fluorophenyl)ethanone (98%, 1.00 g, 3.90 mmol) in anhydrous DCM (8.6 mL) at room temperature under nitrogen were added successively 2 M dimethylamine in THF (3.5 mL, 7.00 mmol), triethylamine (0.60 mL, 4.30 mmol) and DMAP (24 mg, 0.195 mmol). The reaction mixture was stirred at room temperature overnight. The reaction mixture was quenched with water, and ethyl acetate was added. The aqueous layer was extracted twice with ethyl acetate. The combined organic layers were washed with a saturated aqueous solution of sodium chloride, dried over sodium sulfate, filtered, and concentrated under reduced pressure. The crude was purified by flash chromatography on silica gel using a gradient of MeOH in DCM from 0.2% to 5% to afford the title compound as a brown oil (618 mg, 70% yield, tᵣ=0.47 min). LCMS (Method F): m/z found 216.2 [M+H]⁺; ¹H-NMR (DMSO-d₆, 400 MHz) δ (ppm) 8.18 (dd, J=7.3, 2.2 Hz, 1H), 8.02 (ddd, J=8.6, 4.8, 2.2 Hz, 1H), 7.60-7.54 (m, 1H), 3.74 (s, 2H), 2.24 (s, 6H).

### Step 2: Synthesis of 1-(3-chloro-4-fluoro-phenyl)-2-(dimethylamino)ethanone oxime

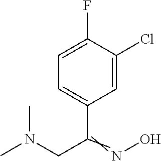

A sealed vial was charged with 1-(3-chloro-4-fluoro-phenyl)-2-(dimethylamino)ethanone (618 mg, 2.87 mmol), hydroxylamine hydrochloride (398 mg, 5.73 mmol) and DIPEA (1.1 mL, 6.02 mmol) in anhydrous ethanol (12 mL). The reaction mixture was stirred at 80° C. for 5 h. The reaction mixture was concentrated under reduced pressure and dried under vacuum to afford the title compound as a brown oil (1.75 g, quantitative yield, 40% purity, tᵣ=0.48). LCMS (Method F): m/z found 231.2 [M+H]⁺.

### Step 3: Synthesis of 1-(3-chloro-4-fluoro-phenyl)-N′,N′-dimethyl-ethane-1,2-diamine

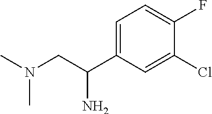

In round-bottom flask, to a stirred solution of 1-(3-chloro-4-fluoro-phenyl)-2-(dimethylamino)ethanone oxime (90%, 796 mg, 3.11 mmol) in anhydrous THF (13.305 mL) at 0° C. under nitrogen was added dropwise 2.4 M LiAlH₄ (3.3 mL, 7.92 mmol) (caution: exothermic reaction with gas evolution). The reaction mixture was stirred at 70° C. for 4 h. The reaction mixture was quenched at 0° C. with water (0.3 mL, same weigh as LiAlH₄, addition dropwise). 1 M sodium hydroxide (0.31 mL, 0.30 mmol) and water (three times the LiAlH₄ weight, 0.9 mL) were added. The resulting heterogenous mixture was stirred at room temperature for 15-30 min, filtered, and rinsed with THF. The filtrate was dried over Na₂SO₄ and concentrated under reduced pressure. The crude was purified by flash chromatography on silica gel using a gradient of MeOH (0.7N NH₃) in DCM from 0.5% to 10%. The desired fractions were combined and concentrated to afford the title compound as a yellow oil (161.5 mg, 23% yield, tᵣ=0.36 min). LCMS (Method F): m/z found 217.2 [M+H]⁺; ¹H-NMR (400 MHz, DMSO-d₆) δ (ppm) 7.58 (dd, J=7.5, 2.1 Hz, 1H), 7.40-7.26 (m, 2H), 3.97 (dd, J=9.0, 5.2 Hz, 1H), 2.26 (dd, J=11.9, 9.0 Hz, 1H), 2.20-2.11 (m, 7H), 1.92 (s, 2H).

### Step 4: Synthesis of N-(1-(3-chloro-4-fluorophenyl)-2-(dimethylamino)ethyl)-4-(trifluoromethoxy)benzenesulfonamide (167)

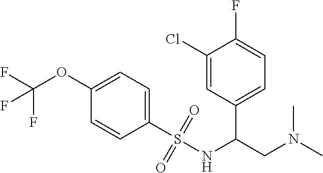 [SMILES: CN(C)CC(NS(=O)(=O)c1ccc(OC(F)(F)F)cc1)c1ccc(F)c(Cl)c1]

In a sealed tube, 4-(trifluoromethoxy)benzenesulfonyl chloride (98%, 41 μL, 0.234 mmol) was added to a stirred solution of 1-(3-chloro-4-fluoro-phenyl)-N′,N′-dimethyl-ethane-1,2-diamine (51 mg, 0.234 mmol) and triethylamine (98 μL, 0.702 mmol) in anhydrous DCM (2.028 mL). The solution was stirred at room temperature overnight. The reaction mixture was diluted with DCM. The organic layer was washed once with a saturated aqueous solution of NH₄Cl, once with a saturated solution of NaHCO₃, and once with a saturated aqueous solution of sodium chloride. The organic layer was dried trough a phase separator and concentrated under reduced pressure. The crude was purified by flash chromatography on silica gel using a gradient of methanol in dichloromethane from 0.5% to 5%. The residue was triturated in pentane, filtered, washed with pentane and dried under vacuum at 45° C. for 20 h to afford the title compounds as white powder (53.3 mg, 510% yield, 99.1% purity, tᵣ=1.38 min). LCMS (Method C): m/z found 441.2 [M+H]⁺; ¹H-NMR (DMSO-d₆, 500 MHz): δ (ppm) 8.30 (br s, 1H), 7.69 (d, J=8.0 Hz, 2H), 7.37 (d, J=8.1 Hz, 2H), 7.28 (d, J=7.3 Hz, 1H), 7.11-7.17 (m, 2H), 4.36 (t, J=7.3 Hz, 1H), 2.42 (dd, J=12.6, 8.4 Hz, 1H), 2.27 (dd, J=12.6, 6.7 Hz, 1H), 2.05 (s, 6H).

### Example 54: N-(1-(4-chloro-3-fluorophenyl)-2-(dimethylamino)ethyl)-4-(trifluoromethoxy)benzenesulfonamide (168)

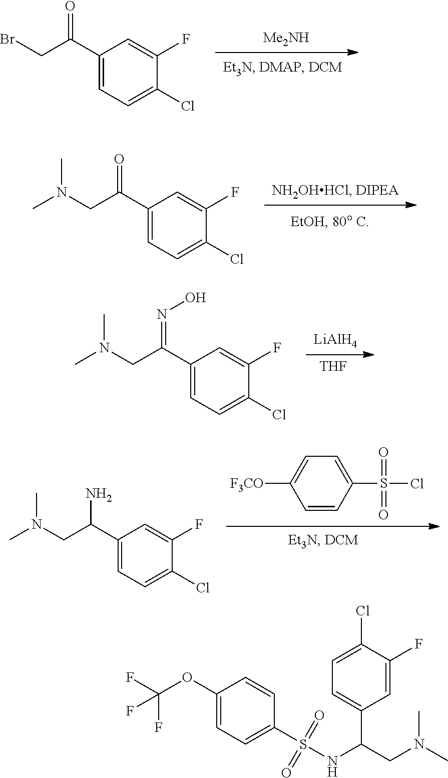

### Step 1: Synthesis of 1-(4-chloro-3-fluoro-phenyl)-2-(dimethylamino)ethanone

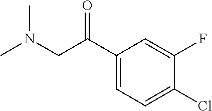

(4-chloro-3-fluorophenyl)ethan-1-one (1.00 g, 3.98 mmol). The reaction mixture was stirred at room temperature for 18 h. The reaction mixture was quenched with water (25 mL) and ethyl acetate (35 mL) was added. The aqueous layer was extracted with ethyl acetate (1×15 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 2% to 500 to afford the title compound as a brown oil (755 mg, 84% yield, tᵣ=0.63 min) LCMS (Method F): m/z found 216.2 [M+H]⁺; ¹H-NMR (DMSO-d₆, 400 MHz): γ (ppm) 7.96 (dd, J=10.1, 1.9 Hz, 1H), 7.84 (ddd, J=8.4, 1.9, 0.4 Hz, 1H), 7.76 (dd, J=8.3, 7.3 Hz, 1H), 3.75 (s, 2H), 2.24 (s, 6H).

### Step 2: Synthesis of 1-(4-chloro-3-fluoro-phenyl)-2-(dimethylamino)ethanone oxime

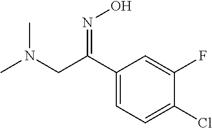

A sealed vial was charged with 1-(4-chloro-3-fluoro-phenyl)-2-(dimethylamino)ethanone (752 mg, 3.49 mmol), hydroxylamine hydrochloride (485 mg, 6.97 mmol) and DIPEA (1.3 mL, 7.32 mmol) in anhydrous ethanol (16 mL). The reaction mixture was stirred at 80° C. for 5 h. The reaction mixture was concentrated under reduced pressure and dried under vacuum to afford the title compound as a brown oil (1.919 g, quantitative yield, tᵣ=0.68 min). LCMS (Method F): m/z found 231.2 [M+H]⁺; ¹H-NMR (DMSO-d₆, 400 MHz) δ (ppm) 9.35 (s, 1H), 7.78-7.48 (m, 3H), 3.67-3.53 (m, 2H), 2.34 (s, 6H).

### Step 3: Synthesis of 1-(4-chloro-3-fluoro-phenyl)-N′,N′-dimethyl-ethane-1,2-diamine

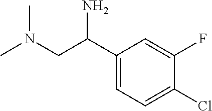

In a vial under atmospheric pressure, to a stirred solution of 1-(4-chloro-3-fluoro-phenyl)-2-(dimethylamino)ethanone oxime (804 mg, 3.49 mmol) in anhydrous THF (15.118 mL) at 0° C. under nitrogen was added 2.4 M LAH in THF (3.6 mL, 8.71 mmol). The reaction mixture was stirred at 70° C. for 3 h. The reaction mixture was quenched at 0° C. with water (0.33 mL, same weight as LAH used), 1 M sodium hydroxide (350 μL, 0.350 mmol) was added and water (three times the weight of LAH used). The resulting heterogenous mixture was stirred at room temperature for 15 min, dried over sodium sulfate, filtered, rinsed with THF, and concentrated under reduced pressure. The crude was purified by flash chromatography on silica gel using a gradient of ammoniacal methanol in dichloromethane from 3% to 8% to afford the title compound as a yellow oil (500 mg, 66% yield, tᵣ=0.36 min). LCMS (Method F): m/z found 217.3 [M+H]⁺; ¹H-NMR (DMSO-d₆, 400 MHz) δ (ppm) 7.49 (t, J=8.0 Hz, 1H), 7.42 (dd, J=10.9, 1.9 Hz, 1H), 7.24 (dd, J=8.3, 1.9 Hz, 1H), 3.98 (dd, J=8.9, 5.3 Hz, 1H), 2.30-2.18 (m, 2H), 2.17 (s, 6H), 1.93 (s, 2H).

### Step 4: Synthesis of N-(1-(4-chloro-3-fluorophenyl)-2-(dimethylamino)ethyl)-4-(trifluoromethoxy)benzenesulfonamide (168)

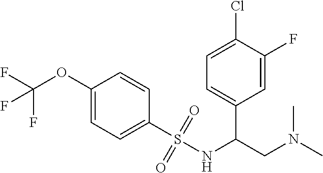 [SMILES: CN(C)CC(NS(=O)(=O)c1ccc(OC(F)(F)F)cc1)c1ccc(Cl)c(F)c1]

In a sealed tube, 4-(trifluoromethoxy)benzenesulfonyl chloride (52 μL, 0.305 mmol) was added to a stirred solution of 1-(4-chloro-3-fluoro-phenyl)-N′,N′-dimethyl-ethane-1,2-diamine (60 mg, 0.277 mmol) and triethylamine (116 μL, 0.831 mmol) in DCM (2.7 mL). The solution was stirred at room temperature for 18 h. Water (5 mL) was added and the aqueous layer was extracted with dichloromethane (1×10 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was triturated in pentane, filtered, washed with pentane and dried under vacuum at 50° C. for 3 days to afford the title compound as a white powder (81.8 mg, 67% yield, 100% purity, tᵣ=1.42 min). LCMS (Method C): m/z found 441 [M+H]⁺; ¹H-NMR (DMSO-d₆, 500 MHz): δ (ppm) 8.33 (br s, 1H), 7.70 (d, J=8.8 Hz, 2H), 7.38 (dd, J=8.9, 0.9 Hz, 2H), 7.31 (t, J=7.9 Hz, 1H), 7.13 (dd, J=10.5, 2.0 Hz, 1H), 7.01 (dd, J=8.3, 2.0 Hz, 1H), 4.36 (t, J=7.3 Hz, 1H), 2.42 (dd, J=12.5, 8.3 Hz, 1H), 2.28 (dd, J=12.6, 6.7 Hz, 1H), 2.05 (s, 6H).

### Example 55: N-(1-(3,4-dichlorophenyl)-2-morpholinoethyl)-4-(trifluoromethoxy)benzenesulfonamide (169)

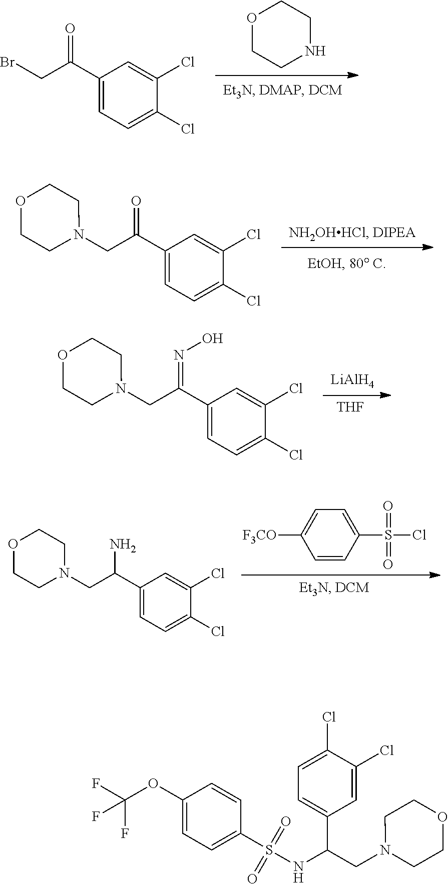

### Step 1: Synthesis of 1-(3,4-dichlorophenyl)-2-morpholino-ethanone

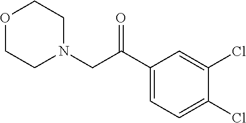 [SMILES: O=C(CN1CCOCC1)c1ccc(Cl)c(Cl)c1]

In a round-bottomed flask, to a stirred solution of 2-bromo-1-(3,4-dichlorophenyl)ethanone (97%, 1.00 g, 3.73 mmol) in anhydrous DCM (8 mL) at room temperature under nitrogen were added successively morpholine (588 μL, 6.72 mmol), triethylamine (572 μL, 4.11 mmol) and DMAP (23 mg, 0.187 mmol). The reaction mixture was stirred at room temperature for 18 h. The reaction mixture was quenched with water (500 mL) and ethyl acetate (800 mL) was added. The aqueous layer was extracted with ethyl acetate (2×30 mL). The combined organic layers were washed with a saturated aqueous solution of sodium chloride, dried over sodium sulfate, filtered and concentrated under reduced pressure. The crude was purified by flash chromatography on silica gel using a gradient of ethyl acetate in cyclohexane from 20% to 100% to afford the title compound as a yellow powder (900 mg, 88% yield, tᵣ=0.51 min). LCMS (Method F): m/z found 274.2 [M+H]⁺; ¹H-NMR (DMSO-d₆, 400 MHz): δ (ppm) 8.18 (d, J=2.0 Hz, 1H), 7.95 (dd, J=8.4, 2.0 Hz, 1H), 7.81 (d, J=8.4 Hz, 1H), 3.86 (s, 2H), 3.61-3.54 (m, 4H), 2.49 (d, J=5.1 Hz, 4H).

### Step 2: Synthesis of 1-(3,4-dichlorophenyl)-2-morpholino-ethanone oxime

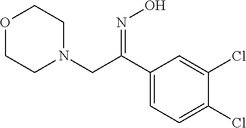 [SMILES: O/N=C(/CN1CCOCC1)c1ccc(Cl)c(Cl)c1]

A sealed vial was charged with 1-(3,4-dichlorophenyl)-2-morpholino-ethanone (900 mg, 3.28 mmol), hydroxylamine hydrochloride (456 mg, 6.57 mmol), and DIPEA (1.2 mL, 6.89 mmol) in anhydrous ethanol (15 mL). The reaction mixture was stirred at 80° C. for 18 h. The reaction mixture was concentrated under reduced pressure and dried under vacuum to afford the title compound as a yellow oil (2.118 g, quantitative yield, tᵣ=0.52 min). LCMS (Method F): m/z found 289.2 [M+H]⁺; ¹H-NMR (DMSO-d₆, 400 MHz) δ (ppm) 9.46 (d, J=51.5 Hz, 1H), 7.92 (dd, J=26.1, 1.9 Hz, 1H), 7.75-7.66 (m, 1H), 7.66-7.58 (m, 1H), 3.63 (s, 2H), 3.52-3.47 (m, 4H), 2.43-2.34 (m, 4H).

### Step 3: Synthesis of 1-(3,4-dichlorophenyl)-2-morpholino-ethanamine

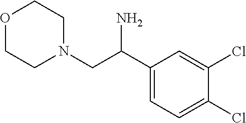 [SMILES: NC(CN1CCOCC1)c1ccc(Cl)c(Cl)c1]

In a vial under atmospheric pressure, to a stirred solution of 1-(3,4-dichlorophenyl)-2-morpholino-ethanone oxime (949 mg, 3.28 mmol) in anhydrous THF (14.5 mL) at 0° C. under nitrogen was added 2.4 M LAH in THF (3.4 mL, 8.20 mmol). The reaction mixture was stirred at 70° C. for 3 h. The reaction mixture was quenched at 0° C. with water (0.31 mL, same weight as LAH used), 1 M sodium hydroxide (0.33 mL, 0.329 mmol) was added and water (three times the weight of LAH used). The resulting heterogenous mixture was stirred at room temperature for 15 min, dried over sodium sulfate, filtered, rinsed with THF, and concentrated under reduced pressure. The crude was purified by flash chromatography on silica gel using a gradient of ammoniacal methanol in dichloromethane from 1% to 5% to afford the expected compound as a yellow oil (554 mg, 61% yield, tᵣ=0.48 min). LCMS (Method F): m/z found 275.2 [M+H]⁺; ¹H-NMR (DMSO-d₆, 400 MHz) δ (ppm) 7.66 (d, J=2.0 Hz, 1H), 7.55 (d, J=8.3 Hz, 1H), 7.37 (dd, J=8.3, 2.0 Hz, 1H), 4.07 (dd, J=8.5, 5.6 Hz, 1H), 3.65-3.50 (m, 4H), 2.49-2.32 (m, 4H), 2.32-2.25 (m, 2H), 2.13 (s, 2H).

### Step 4: Synthesis of N-(1-(3,4-dichlorophenyl)-2-morpholinoethyl)-4-(trifluoromethoxy)benzenesulfonamide (169)

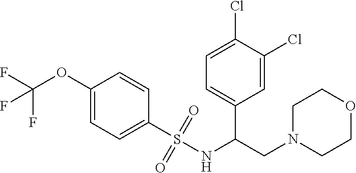 [SMILES: O=S(=O)(NC(CN1CCOCC1)c1ccc(Cl)c(Cl)c1)c1ccc(OC(F)(F)F)cc1]

In a sealed tube, 4-(trifluoromethoxy)benzenesulfonyl chloride (41 μL, 0.240 mmol) was added to a stirred solution of 1-(3,4-dichlorophenyl)-2-morpholino-ethanamine (60 mg, 0.218 mmol) and triethylamine (91 μL, 0.654 mmol) in anhydrous DCM (2.2 mL). The solution was stirred at room temperature for 18 h. Water (5 mL) was added and the aqueous layer was extracted with ethyl acetate (1×10 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was triturated in pentane, filtered, washed with pentane and dried under vacuum at 70° C. for 18 h to afford the title compound as a white powder (88.4 mg, 81% yield, 99.8% purity, tᵣ=2.1 min). LCMS (Method C): m/z found 499 [M+H]⁺; ¹H-NMR (DMSO-d₆, 500 MHz): δ (ppm) 8.35 (d, J=7.8 Hz, 1H), 7.76 (d, J=8.8 Hz, 2H), 7.40-7.44 (m, 4H), 7.20 (dd, J=8.3, 2.0 Hz, 1H), 4.43 (br d, J=5.9 Hz, 1H), 3.33-3.43 (m, 4H), 2.45-2.49 (m, 1H), 2.22-2.37 (m, 5H).

### Example 56: N-(1-(3,4-dichlorophenyl)-2-(dimethylamino)ethyl)-4-phenoxybenzenesulfonamide (170 and 171

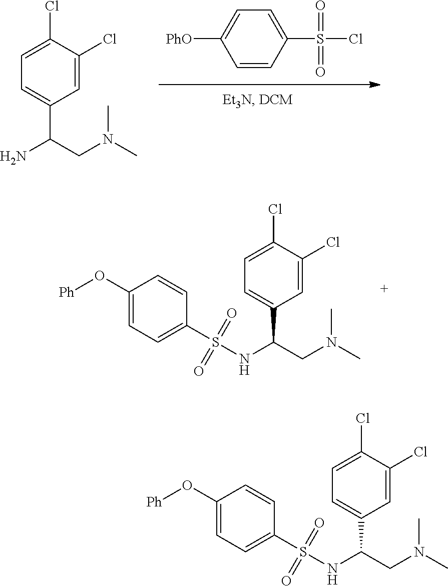 [SMILES: CN(C)CC(N)c1ccc(Cl)c(Cl)c1 ; O=S(=O)(Cl)c1ccc(Oc2ccccc2)cc1 ; CN(C)C[C@@H](NS(=O)(=O)c1ccc(Oc2ccccc2)cc1)c1ccc(Cl)c(Cl)c1 ; CN(C)C[C@H](NS(=O)(=O)c1ccc(Oc2ccccc2)cc1)c1ccc(Cl)c(Cl)c1]

In a sealed tube, 4-phenoxybenzenesulfonyl chloride (565 mg, 2.10 mmol) was added to a stirred solution of 1-(3,4-dichlorophenyl)-N′,N′-dimethyl-ethane-1,2-diamine (98%, 500 mg, 2.10 mmol) and triethylamine (879 μL, 6.31 mmol) in anhydrous DCM (22 mL). The solution was stirred at room temperature for 20 h. Water (5 mL) was added and the aqueous layer was extracted with dichloromethane (1×5 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was subjected to chiral separation each batch was purified by flash chromatography on silica gel using a gradient of methanol in dichloromethane from 0% to 5%. The residues were triturated in pentane, filtered, washed with pentane and dried under vacuum at 70° C. for 18 h to afford a racemic mixture of the title compounds as a white powder. 718 mg of the racemic mixture was dissolved by sonication in 26 mL (MeOH+0.5% i-PrNH₂) to provide a solution having a concentration of 27.6 mg/mL. The solution was passed through a GHP filter (0.45 μm). 13 injections of the sample solution were performed on a Chiralpak AD-H column (250×20 mm, 5 μm) using 25% (MeOH+0.5% i-PrNH₂) in CO₂ as a mobile phase (Waters Prep SFC80, flow rate=50 mL/min, T=40° C., P=100 bar, UV detection at 220 nm) to afford the separated enantiomers.

Enantiomer I (170): (160.7 mg, 16% yield, 100% purity, tᵣ=1.81 min). LCMS (Method C): m/z found 465 [M+H]⁺; ¹H-NMR (DMSO-d₆, 500 MHz): δ (ppm) 8.09 (br d, J=7.6 Hz, 1H), 7.56 (d, J=8.8 Hz, 2H), 7.47 (dd, J=8.4, 7.5 Hz, 2H), 7.42 (d, J=8.3 Hz, 1H), 7.35 (d, J=2.0 Hz, 1H), 7.25 (t, J=7.5 Hz, 1H), 7.16 (dd, J=8.3, 2.2 Hz, 1H), 7.01-7.06 (m, 2H), 6.84-6.95 (m, 2H), 4.20-4.44 (m, 1H), 2.25-2.45 (m, 2H), 2.07 (s, 6H).

Enantiomer II (171): (119.5 mg, 12% yield, 100% purity, tᵣ=1.81 min). LCMS (Method C): m/z found 465 [M+H]⁺; ¹H-NMR (DMSO-d₆, 500 MHz): δ (ppm) 8.09 (br d, J=4.6 Hz, 1H), 7.56 (d, J=8.8 Hz, 2H), 7.47 (dd, J=8.4, 7.5 Hz, 2H), 7.42 (d, J=8.1 Hz, 1H), 7.35 (d, J=2.0 Hz, 1H), 7.23-7.27 (m, 1H), 7.16 (dd, J=8.3, 2.2 Hz, 1H), 7.04 (dd, J=8.6, 1.0 Hz, 2H), 6.89 (d, J=8.8 Hz, 2H), 4.33 (br dt, J=7.8 Hz, 1H), 2.40 (dd, J=12.2, 8.1 Hz, 1H), 2.30 (dd, J=12.5, 7.1 Hz, 1H), 2.07 (s, 6H).

### Example 57: N-(1-(3,4-dichlorophenyl)-2-(dimethylamino)ethyl)-4-(4-(trifluoromethyl)phenoxy)benzenesulfonamide (172 and 173

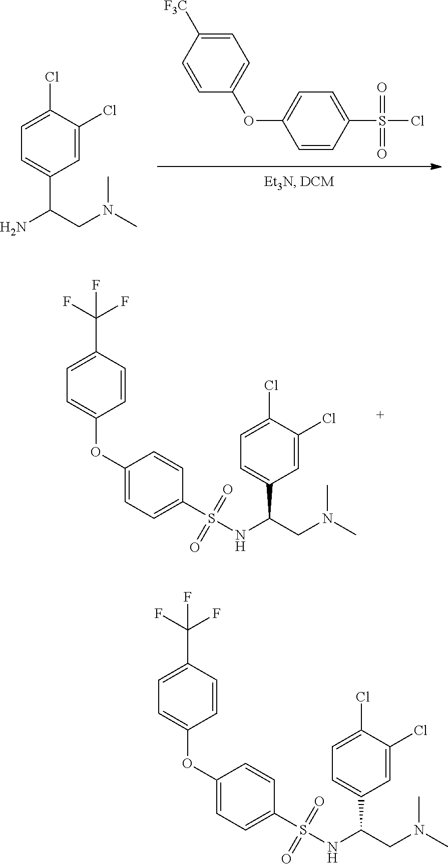 [SMILES: CN(C)CC(N)c1ccc(Cl)c(Cl)c1 ; O=S(=O)(Cl)c1ccc(Oc2ccc(C(F)(F)F)cc2)cc1 ; CN(C)C[C@@H](NS(=O)(=O)c1ccc(Oc2ccc(C(F)(F)F)cc2)cc1)c1ccc(Cl)c(Cl)c1 ; CN(C)C[C@H](NS(=O)(=O)c1ccc(Oc2ccc(C(F)(F)F)cc2)cc1)c1ccc(Cl)c(Cl)c1]

In a sealed tube, triethylamine (1.1 mL, 7.80 mmol) was added to a stirred solution of 4-[4-(trifluoromethyl)phenoxy]benzenesulfonyl chloride (657 mg, 1.95 mmol) and 1-(3,4-dichlorophenyl)-N²,N²-dimethylethane-1,2-diamine (500 mg, 2.15 mmol) in DCM (10.526 mL). The solution was stirred at rt for 18 h and was diluted with an half saturated aqueous solution of NaHCO₃. The aqueous layer was extracted with DCM, the combined organic layers were washed with brine, dried over sodium sulfate, filtered and concentrated. The crude was purified by flash chromatography on silica gel using a gradient of (MeOH+2% NH₄OH) in DCM from 0.2% to 5% to obtain the racemic product as a yellow foam (750 mg, 72% yield). This racemic mixture was purified by chiral SFC using CO₂/(EtOH+0.5% i-PrNH₂) as mobile phase to afford two fractions. Each fraction was dissolved in DCM, washed with half saturated aqueous NaHCO₃ and brine, dried over sodium sulfate, filtered and concentrated. Each residue was dissolved in Et₂O and added dropwise to stirred 2 N HCl/Et₂O. Each suspension was stirred at rt for 16 h and concentrated to afford a racemic mixture of the title compounds as a white powder. 750 mg of the racemic mixture was dissolved by sonication in 9 mL of (EtOH+0.5% i-PrNH₂) to provide a solution having a concentration of 83.33 mg/mL. The solution was passed through a GHP filter (0.45 μm). 18 injections of the sample solution were performed on a Chiralpak AD-H column (250×20 mm, 5 μm) using 25% (EtOH+0.5% i-PrNH₂) in CO₂ as a mobile phase (Waters Prep SFC80, flow rate=50 mL/min, T=40° C., P=100 bar, UV detection at 210 nm) to afford the separated enantiomers.

Enantiomer I (172): (202.1 mg, 18% yield, 99.4% purity, tᵣ=2.26 min). LCMS (Method D): m/z found 533 [M+H]⁺; ¹H-NMR (500 MHz, DMSO-d₆, 300 K) δ (ppm) 9.68 (br s, 1H), 8.80 (br d, J=10.5 Hz, 1H), 7.83 (d, J=8.6 Hz, 2H), 7.61-7.54 (m, 2H), 7.52-7.48 (m, 2H), 7.27 (dd, J=2.0, 8.3 Hz, 1H), 7.13 (d, J=8.6 Hz, 2H), 7.01-6.93 (m, 2H), 4.86 (dt, J=3.9, 10.5 Hz, 1H), 3.42-3.36 (m, 1H), 3.29-3.18 (m, 1H), 3.03-2.66 (m, 6H).

Enantiomer II (173): (241.8 mg, 22% yield, 99.8% purity, tᵣ=2.26 min). LCMS (Method D): m/z found 533 [M+H]⁺; ¹H-NMR (500 MHz, DMSO-d₆, 300 K) δ (ppm) 9.68 (br s, 1H), 8.80 (br d, J=10.5 Hz, 1H), 7.83 (d, J=8.6 Hz, 2H), 7.61-7.54 (m, 2H), 7.52-7.48 (m, 2H), 7.27 (dd, J=2.0, 8.3 Hz, 1H), 7.13 (d, J=8.6 Hz, 2H), 7.01-6.93 (m, 2H), 4.86 (dt, J=3.9, 10.5 Hz, 1H), 3.42-3.36 (m, 1H), 3.29-3.18 (m, 1H), 3.03-2.66 (m, 6H).

### Example 58: (E)-N-(1-(3,4-dichlorophenyl)-2-(dimethylamino)ethyl)-2-phenylethene-1-sulfonamide (174)

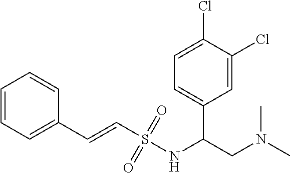 [SMILES: CN(C)CC(NS(=O)(=O)/C=C/c1ccccc1)c1ccc(Cl)c(Cl)c1]

In a sealed tube, (E)-2-phenylethenesulfonyl chloride (78 μL, 0.592 mmol) was added to a stirred solution of 1-(3,4-dichlorophenyl)-N²,N²-dimethylethane-1,2-diamine (152 mg, 0.651 mmol) and pyridine (96 μL, 1.18 mmol) in THF (3.5 mL). The solution was stirred at rt for 18 h and was diluted with an half saturated aqueous solution of NaHCO₃. The aqueous layer was extracted with DCM, the combined organic layers were washed with a saturated aqueous solution of NaHCO₃, brine, dried over sodium sulfate, filtered and concentrated. The crude was purified by flash chromatography on silica gel using a gradient of (MeOH+2% NH₄OH) in DCM from 0.2% to 5%. The desired fractions were concentrated and pentane was added to the residue. A few drops of Et₂O were added and the obtained suspension was triturated and filtered. The residue was washed with pentane and was dried under reduced pressure at 40° C. for 16 h to afford the title compound as a white powder. (57 mg, 24% yield, 99.7% purity, tᵣ=1.58 min). LCMS (Method C): m/z found 398.9 [M+H]⁺; ¹H-NMR (500 MHz, DMSO-d₆, 300 K) δ (ppm) 7.81 (br s, 1H), 7.61 (d, J=2.0 Hz, 1H), 7.55-7.45 (m, 3H), 7.44-7.37 (m, 3H), 7.34 (dd, J=2.2, 8.3 Hz, 1H), 7.21 (d, J=15.7 Hz, 1H), 6.89 (d, J=15.4 Hz, 1H), 4.37 (br t, J=7.2 Hz, 1H), 2.57-2.51 (m, 1H), 2.37 (dd, J=6.8, 12.5 Hz, 1H), 2.13 (s, 6H).

### Example 59: N-(1-(3,4-dichlorophenyl)-2-(dimethylamino)ethyl)-3-nitro-4-(trifluoromethoxy)benzenesulfonamide (175)

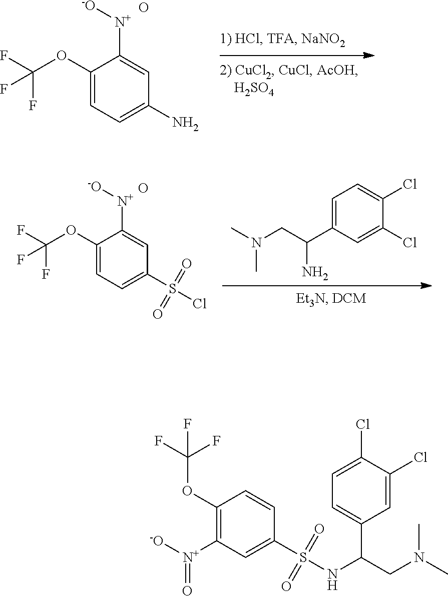

### Step 1: Synthesis of 3-nitro-4-(trifluoromethoxy)benzenesulfonyl chloride

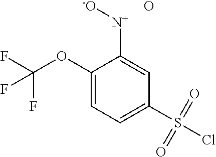

In a three necked round bottomed flask equipped with a thermometer, a solution of 3-nitro-4-(trifluoromethoxy)aniline (98%, 300 mg, 1.32 mmol) in TFA (4.5 mL) was stirred at 0° C. Copper(I) chloride (4.0 mg, 0.0397 mmol) was added, the mixture was stirred at 0° C. for 5 min then a solution of sodium nitrite (116 mg, 1.65 mmol) in water (500 μL) was added dropwise over 10 min to keep the temperature below 0° C. The mixture was stirred at −5° C. for 30 min to afford Solution A. In a three necked round bottomed flask equipped with a thermometer, a solution of copper(II) dichloride (91 mg, 0.675 mmol) and concentrated hydrogen chloride (442 μL, 14.6 mmol) in Acetic acid (4.5 mL) and sulfurous acid (4.5 mL, 58.2 mmol) was stirred at −5° C. The Solution A was added dropwise over 5 min to keep the temperature below 5° C. The mixture was stirred at 0° C. for 5 min then a rt for 1 h and was extracted twice with DCM. The combined organic layers were dried over sodium sulfate, filtered and concentrated to afford the title compound as a red liquid. (330 mg, 81% yield). ¹H-NMR (DMSO-d₆, 400 MHz) δ (ppm) 8.24 (d, J=2.1 Hz, 1H), 8.03 (dd, J=8.5, 2.1 Hz, 1H), 7.76-7.67 (m, 1H).

### Step 2: Synthesis of N-(1-(3,4-dichlorophenyl)-2-(dimethylamino)ethyl)-3-nitro-4-(trifluoromethoxy)benzenesulfonamide (175)

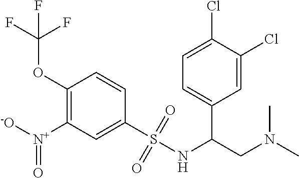

In a sealed tube, 3-nitro-4-(trifluoromethoxy)benzenesulfonyl chloride (35 μL, 0.327 mmol) was added to a stirred solution of 1-(3,4-dichlorophenyl)-N²,N²-dimethylethane-1,2-diamine (84 mg, 0.360 mmol) and triethylamine (182 μL, 1.31 mmol) in DCM (2 mL). The solution was stirred at rt for 18 h and was diluted with an half saturated aqueous solution of NaHCO₃. The aqueous layer was extracted with DCM, the combined organic layers were washed with a saturated aqueous solution of NaHCO₃, brine, dried over sodium sulfate, filtered and concentrated. The crude was purified by flash chromatography on silica gel using a gradient of (MeOH+2% NH₄OH) in DCM from 0.2% to 5%. The desired fractions were concentrated and pentane was added to the residue. A few drops of Et₂O were added and the obtained suspension was triturated and filtered. The residue was washed with pentane and was dried under reduced pressure at 40° C. for 16 h to afford the title product as a beige powder (88 mg, 52% yield, 98.7% purity, tᵣ=1.76 min). LCMS (Method C): m/z found 501.9 [M+H]⁺; ¹H-NMR (500 MHz, DMSO-d₆, 300 K) δ (ppm) 9.15-8.39 (m, 1H), 8.28 (d, J=2.2 Hz, 1H), 8.01 (dd, J=2.3, 8.7 Hz, 1H), 7.79 (dd, J=1.3, 8.7 Hz, 1H), 7.45-7.33 (m, 2H), 7.17 (dd, J=2.1, 8.2 Hz, 1H), 4.45 (dd, J=6.0, 8.9 Hz, 1H), 2.53-2.45 (m, 1H), 2.26 (dd, J=6.0, 12.6 Hz, 1H), 2.05 (s, 6H).

### Example 60: 3-amino-N-(1-(3,4-dichlorophenyl)-2-(dimethylamino)ethyl)-4-(trifluoromethoxy)benzenesulfonamide (176)

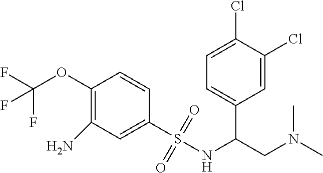 [SMILES: CN(C)CC(NS(=O)(=O)c1ccc(OC(F)(F)F)c(N)c1)c1ccc(Cl)c(Cl)c1]

In a sealed tube, a suspension of iron (28 mg, 0.493 mmol) and ammonium chloride (6.7 mg, 0.125 mmol) in ethanol (600 μL) and water (200 μL) was stirred at 70° C. for 1 h. N-[1-(3,4-dichlorophenyl)-2-(dimethylamino)ethyl]-3-nitro-4-(trifluoromethoxy)benzenesulfonamide (45 mg, 0.0896 mmol) was added and the mixture was stirred at 80° C. for 1 h. The mixture was allowed to cool to rt and was filtered through a pad of dicalite. The pad was washed with EtOH and the filtrate was concentrated. The crude was purified by flash chromatography on silica gel using a gradient of (MeOH+2% NH₄OH) in DCM from 1% to 10%. The desired fractions were concentrated and pentane was added to the residue. A few drops of Et₂O were added and the obtained suspension was triturated and filtered. The residue was washed with pentane and dried under reduced pressure at 35° C. for 16 h to afford the title compound as a white powder (30 mg, 70% yield, 99.4% purity, tᵣ=1.36 min). LCMS (Method C): m/z found 472.2 [M+H]⁺; ¹H-NMR (500 MHz, DMSO-d₆) δ (ppm) 8.02 (br s, 1H), 7.39-7.42 (m, 2H), 7.17 (dd, J=2.20, 8.31 Hz, 1H), 7.08-7.11 (m, 2H), 6.78 (dd, J=2.32, 8.44 Hz, 1H), 5.75 (s, 2H), 4.26 (br t, J=7.34 Hz, 1H), 2.26-2.37 (m, 2H), 2.03 (s, 6H).

### Example 61: N-(2-(dimethylamino)-1-(3-(trifluoromethyl)phenyl)ethyl)-4-(trifluoromethoxy)benzenesulfonamide (177 and 178

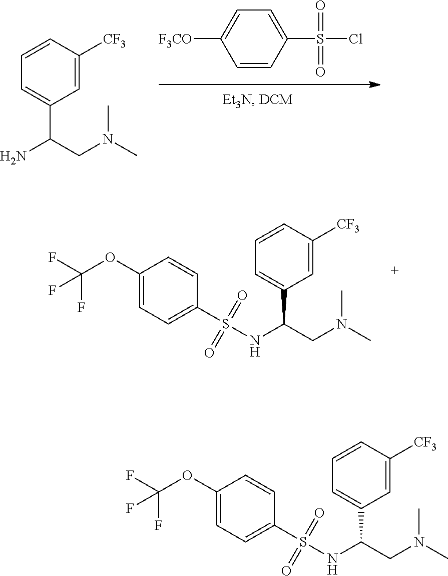 [SMILES: CN(C)CC(N)c1cccc(C(F)(F)F)c1 ; O=S(=O)(Cl)c1ccc(OC(F)(F)F)cc1 ; CN(C)C[C@@H](NS(=O)(=O)c1ccc(OC(F)(F)F)cc1)c1cccc(C(F)(F)F)c1 ; CN(C)C[C@H](NS(=O)(=O)c1ccc(OC(F)(F)F)cc1)c1cccc(C(F)(F)F)c1]

In a sealed vial under nitrogen, N′,N′-dimethyl-1-[3-(trifluoromethyl)phenyl]ethane-1,2-diamine (95%, 500 mg, 2.05 mmol) was added to a stirred solution of 4-(trifluoromethoxy)benzenesulfonyl chloride (0.35 mL, 2.05 mmol) and triethylamine (1.1 mL, 8.18 mmol) in anhydrous DCM (10.226 mL). The solution was stirred at rt overnight, DCM and an half saturated solution of NaHCO₃ were added, the aqueous layer was extracted once more with DCM, organic layers were gathered, concentrated in vacuo and purified by flash chromatography on silica gel using a gradient of MeOH (0.7 N NH₃) in DCM from 0% to 4% to afford the racemic product as a colorless oil (373 mg, 40% yield). 820 mg of the racemic mixture was dissolved by sonication in 19 mL (EtOH+0.5% i-PrNH₂) and 3 mL (ACN), with heating, to provide a solution having a concentration of 37.3 mg/mL. The solution was passed through a GHP filter (0.45 μm). 110 injections of the sample solution were performed on a Chiralsel OJ-H column (250×21 mm, 5 μm) using 17% (EtOH+0.5% i-PrNH₂) in CO₂ as a mobile phase (Waters Prep SFC200, flow rate=50 mL/min, T=40° C., P=150 bar, UV detection at 210 nm) to afford the separated enantiomers. Compound 177 was obtained as a colorless oil with traces of ethanol, dried under vacuum at 50° C., then 2M HCl in diethyl ether was added (2 mL) and the mixture was stirred 5 hours at rt and concentrated in vacuo to afford the enantiomer as a white powder. Compound 178 was obtained as a colorless oil with traces of ethanol, which was dried under vacuum at 50° C., then 2 M HCl in diethyl ether was added (2 mL) and the mixture was stirred 5 hours at rt and concentrated in vacuo to afford the enantiomer as a white powder.

Enantiomer I (177): (95.4 mg, 9.5% yield, 99.8% purity, tᵣ=1.67 min). LCMS (Method C): m/z found 457 [M+H]⁺; ¹H-NMR (DMSO-d₆, 500 MHz): δ (ppm) 9.66 (br s, 1H), 8.96 (br s, 1H), 7.61 (d, J=8.8 Hz, 2H), 7.56 (s, 1H), 7.56 (d, J=9.1 Hz, 1H), 7.44 (d, J=7.8 Hz, 1H), 7.34 (t, J=7.7 Hz, 1H), 7.20 (d, J=8.6 Hz, 2H), 4.97 (t, J=8.6 Hz, 1H), 3.36-3.45 (m, 1H), 3.21-3.29 (m, 1H), 2.93 (br s, 3H), 2.86 (br s, 3H).

Enantiomer II (178): (70.2 mg, 7% yield, 100% purity, tᵣ=1.66 min). LCMS (Method C): m/z found 457 [M+H]⁺; ¹H-NMR (DMSO-d₆, 500 MHz): δ (ppm) 9.70 (br s, 1H), 8.99 (br d, J=9.8 Hz, 1H), 7.61 (d, J=8.8 Hz, 2H), 7.53-7.58 (m, 2H), 7.43 (d, J=7.6 Hz, 1H), 7.31-7.37 (m, 1H), 7.20 (d, J=8.6 Hz, 2H), 4.97 (br t, J=9.5 Hz, 1H), 3.37-3.47 (m, 1H), 3.20-3.29 (m, 1H), 2.93 (br s, 3H), 2.86 (br s, 3H).

### Example 62: N-(1-(4-chlorophenyl)-2-(dimethylamino)ethyl)-4-(trifluoromethoxy)benzenesulfonamide (179 and 180

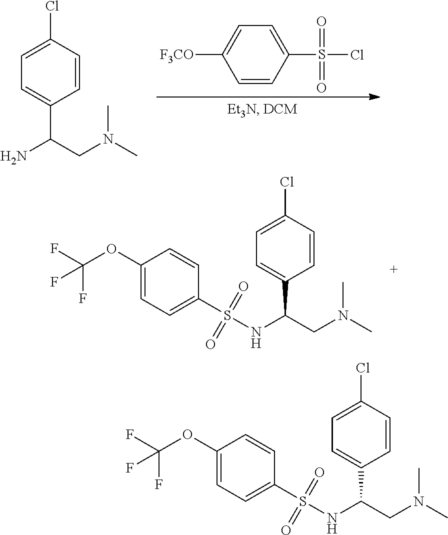

In a sealed tube, 4-(trifluoromethoxy)benzenesulfonyl chloride (35 μL, 2.30 mmol) was added to a stirred solution of 1-(4-chlorophenyl)-N′,N′-dimethyl-ethane-1,2-diamine (503 mg, 2.53 mmol) and triethylamine (1.3 mL, 9.21 mmol) in DCM (14 mL). The solution was stirred at rt for 18 h and was diluted with an half saturated aqueous solution of NaHCO₃. The aqueous layer was extracted with DCM, the combined organic layers were washed brine, dried over sodium sulfate, filtered and concentrated. The crude was purified by flash chromatography on silica gel using a gradient of (MeOH+2% NH₄OH) in DCM from 0.2% to 5% to obtain the racemic product as a yellow solid (735 mg, 75% yield). 735 mg of the racemic mixture was dissolved by sonication in 8 mL (EtOH+0.5% i-PrNH₂) and 3 mL (MeOH) to provide a solution having a concentration of 66.8 mg/mL. The solution was passed through a GHP filter (0.45 μm). 28 injections of the sample solution were performed on a Chiralpak AD-H column (250×20 mm, 5 μm) using 20% (EtOH+0.5% i-PrNH₂) in CO₂ as a mobile phase (Waters Prep SFC80, flow rate=50 mL/min, T=40° C., P=100 bar, UV detection at 220 nm) to afford the separated enantiomers.

Enantiomer I (179): (296 mg, 30% yield, 99.4% purity, tᵣ=1.79 min). LCMS (Method D): m/z found 423 [M+H]⁺; ¹H-NMR (500 MHz, DMSO-d₆, 300 K) δ (ppm) 8.29 (br s, 1H), 7.70 (d, J=8.8 Hz, 2H), 7.37 (d, J=8.1 Hz, 2H), 7.21-7.08 (m, 4H), 4.31 (t, J=7.5 Hz, 1H), 2.41 (dd, J=8.6, 12.5 Hz, 1H), 2.25 (dd, J=6.5, 12.6 Hz, 1H), 2.02 (s, 6H).

Enantiomer II (180): (296 mg, 30% yield, 100% purity, tᵣ=1.8 min). LCMS (Method D): m/z found 423 [M+H]⁺; ¹H-NMR (500 MHz, DMSO-d₆, 300 K) δ (ppm) 8.29 (br s, 1H), 7.70 (d, J=8.8 Hz, 2H), 7.37 (d, J=8.1 Hz, 2H), 7.15 (d, J=5.6 Hz, 3H), 7.20-7.04 (m, 1H), 4.31 (t, J=7.5 Hz, 1H), 2.40 (dd, J=8.6, 12.5 Hz, 1H), 2.25 (dd, J=6.5, 12.6 Hz, 1H), 2.02 (s, 6H).

### Example 63: N-(1-(4-chlorophenyl)-2-(pyrrolidin-1-yl)ethyl)-4-(trifluoromethoxy)benzenesulfonamide (181)

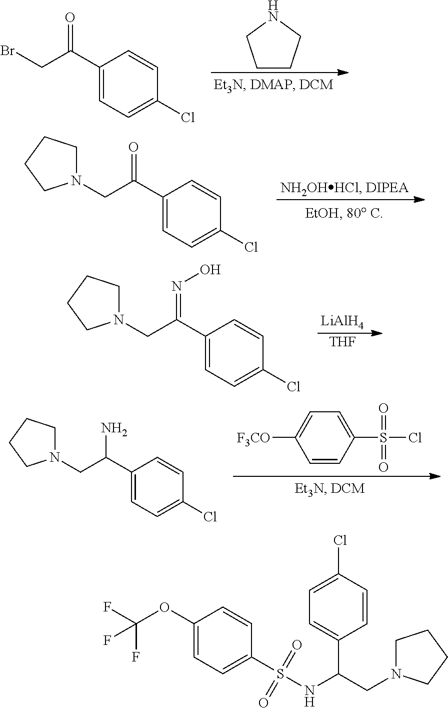

### Step 1: Synthesis of 1-(4-chlorophenyl)-2-pyrrolidin-1-yl-ethanone

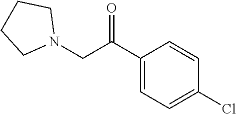 [SMILES: O=C(CN1CCCC1)c1ccc(Cl)cc1]

In a round-bottomed flask, to a stirred solution of 2-bromo-1-(4-chlorophenyl)ethanone (97%, 1.00 g, 4.15 mmol) in anhydrous DCM (9.5 mL) at room temperature under nitrogen were added successively pyrrolidine (624 μL, 7.48 mmol), triethylamine (637 μL, 4.57 mmol) and DMAP (25 mg, 0.208 mmol). The reaction mixture was stirred at room temperature for 18 h. The reaction mixture was quenched with water (25 mL) and dichloromethane (25 mL) was added. The aqueous layer was extracted with dichloromethane (2×15 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5% to afford the title compound as an orange powder (754 mg, 76% yield, tᵣ=0.65 min). LCMS (Method F): m/z found 224.2 [M+H]⁺; ¹H-NMR (DMSO-d₆, 400 MHz) δ (ppm) 8.03-7.98 (m, 2H), 7.63-7.55 (m, 2H), 3.94 (s, 2H), 2.56 (t, J=5.7 Hz, 4H), 1.75-1.64 (m, 4H).

### Step 2: Synthesis of 1-(4-chlorophenyl)-2-pyrrolidin-1-yl-ethanone oxime

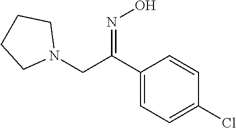 [SMILES: O/N=C(/CN1CCCC1)c1ccc(Cl)cc1]

A sealed vial was charged with 1-(4-chlorophenyl)-2-pyrrolidin-1-yl-ethanone (750 mg, 3.35 mmol), hydroxylamine hydrochloride (466 mg, 6.71 mmol) and DIPEA (1.2 mL, 7.04 mmol) in anhydrous ethanol (15 mL). The reaction mixture was stirred at 80° C. for 18 h. The reaction mixture was quenched with a saturated aqueous solution of ammonium chloride (2 mL) and water (20 mL) and ethyl acetate (20 mL) were added. The aqueous layer was extracted with ethyl acetate (1×20 mL). The combined organic layers were washed with a saturated aqueous solution of sodium chloride, dried over sodium sulfate, filtered and concentrated under reduced pressure to afford the title compound as a white gum (768 mg, 72% yield, tᵣ=0.69 min). LCMS (Method F): m/z found 239.3 [M+H]⁺; ¹H-NMR (DMSO-d₆, 400 MHz) δ (ppm) 7.78-7.72 (m, 2H), 7.42 (dt, J=9.3, 2.2 Hz, 2H), 3.72 (s, 2H), 2.46 (s, 4H), 1.63 (t, J=3.3 Hz, 4H).

### Step 3: Synthesis of 1-(4-chlorophenyl)-2-pyrrolidin-1-yl-ethanamine

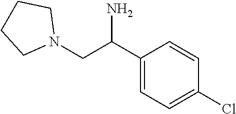 [SMILES: NC(CN1CCCC1)c1ccc(Cl)cc1]

In a vial under atmospheric pressure, to a stirred solution of 1-(4-chlorophenyl)-2-pyrrolidin-1-yl-ethanone oxime (765 mg, 3.20 mmol) in anhydrous (13.9 mL) at 0° C. under nitrogen was added 2.4 M LAH in THF (3.3 mL, 8.01 mmol). The reaction mixture was stirred at 70° C. for 3 h. The reaction mixture was quenched at 0° C. with water (0.31 mL, same weight as LAH used), 1 M sodium hydroxide (0.32 mL, 0.322 mmol) was added and water (three times the weight of LAH used). The resulting heterogenous mixture was stirred at room temperature for 15 min, dried over sodium sulfate, filtered, rinsed with THF, and concentrated under reduced pressure. The crude was purified by flash chromatography on silica gel using a gradient of ammoniacal methanol in dichloromethane from 3% to 8% to afford the title compound as a yellow oil (282.3 mg, 36% yield, tᵣ=0.39 min). LCMS (Method F): m/z found 225.3 [M+H]⁺; ¹H-NMR (DMSO-d₆, 400 MHz) δ (ppm) 7.43-7.37 (m, 2H), 7.37-7.31 (m, 2H), 3.96 (dd, J=9.1, 5.0 Hz, 1H), 2.54-2.48 (m, 3H), 2.46-2.38 (m, 2H), 2.31 (dd, J=11.7, 5.0 Hz, 1H), 1.92 (s, 2H), 1.73-1.62 (m, 4H).

### Step 4: Synthesis of N-(1-(4-chlorophenyl)-2-(pyrrolidin-1-yl)ethyl)-4-(trifluoromethoxy)benzenesulfonamide (181)

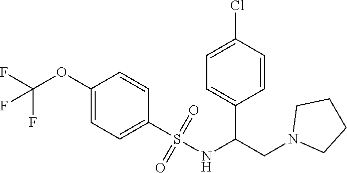 [SMILES: O=S(=O)(NC(CN1CCCC1)c1ccc(Cl)cc1)c1ccc(OC(F)(F)F)cc1]

In a sealed tube, 4-(trifluoromethoxy)benzenesulfonyl chloride (45 μL, 0.267 mmol) was added to a stirred solution of 1-(4-chlorophenyl)-2-pyrrolidin-1-yl-ethanamine (60 mg, 0.267 mmol) and triethylamine (112 μL, 0.801 mmol) in anhydrous DCM (2.7 mL). The solution was stirred at room temperature for 18 h. Water (5 mL) was added and the aqueous layer was extracted with dichloromethane (1×10 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was triturated in pentane, filtered, washed with pentane and dried under vacuum at 70° C. for 3 days to afford the title compound as a white powder (83.9 mg, 68% yield, 96.5% purity, tᵣ=1.47 min). LCMS (Method C): m/z found 449.2 [M+H]⁺; ¹H-NMR (DMSO-d₆, 500 MHz): δ (ppm) 8.32 (br s, 1H), 7.73 (d, J=8.8 Hz, 2H), 7.39 (d, J=8.3 Hz, 2H), 7.13-7.21 (m, 4H), 4.29 (t, J=6.6 Hz, 1H), 2.63 (br dd, J=11.5, 9.3 Hz, 1H), 2.41 (br dd, J=12.3, 6.2 Hz, 1H), 2.20-2.38 (m, 4H), 1.45-1.61 (m, 4H).

### Example 64: Synthesis of N-(1-(3,4-dichlorophenyl)-2-(pyrrolidin-1-yl)ethyl)-4-(trifluoromethoxy)benzenesulfonamide (182)

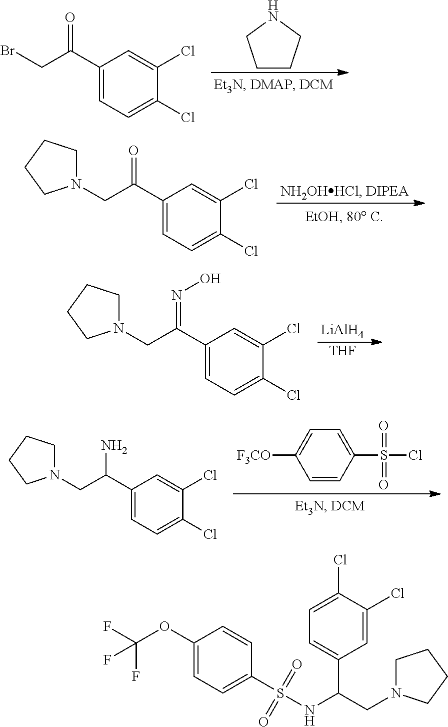

### Step 1: Synthesis of 1-(3,4-dichlorophenyl)-2-pyrrolidin-1-yl-ethanone

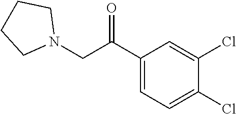 [SMILES: O=C(CN1CCCC1)c1ccc(Cl)c(Cl)c1]

In a round-bottomed flask, to a stirred solution of 2-bromo-1-(3,4-dichlorophenyl)ethanone (97%, 1.00 g, 3.62 mmol) in anhydrous DCM (8 mL) at room temperature under nitrogen were added successively pyrrolidine (544 μL, 6.52 mmol), triethylamine (555 μL, 3.98 mmol) and DMAP (22 mg, 0.181 mmol). The reaction mixture was stirred at room temperature for 18 h. The reaction mixture was quenched with water (25 mL) and dichloromethane (25 mL) was added. The aqueous layer was extracted with dichloromethane (2×15 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5% to afford the title compound as a brown oil (654.3 mg, 64% yield, tᵣ=0.79 min). LCMS (Method F): m/z found 258.2 [M+H]⁺; ¹H-NMR (DMSO-d₆, 400 MHz) δ (ppm) 8.17 (d, J=2.0 Hz, 1H), 7.95 (dd, J=8.4, 2.0 Hz, 1H), 7.80 (d, J=8.4 Hz, 1H), 3.98 (s, 2H), 2.62-2.53 (m, 4H), 1.74-1.67 (m, 4H).

### Step 2: Synthesis of 1-(3,4-dichlorophenyl)-2-pyrrolidin-1-yl-ethanone oxime

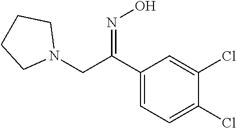 [SMILES: O/N=C(/CN1CCCC1)c1ccc(Cl)c(Cl)c1]

A sealed vial was charged with 1-(3,4-dichlorophenyl)-2-pyrrolidin-1-yl-ethanone (650 mg, 2.52 mmol), hydroxylamine hydrochloride (350 mg, 5.04 mmol) and DIPEA (924 μL, 5.29 mmol) in anhydrous ethanol (11 mL). The reaction mixture was stirred at 80° C. for 18 h. The reaction mixture was quenched with a saturated aqueous solution of ammonium chloride (2 mL) and water (20 mL) and ethyl acetate (20 mL) were added. The aqueous layer was extracted with ethyl acetate (1×20 mL). The combined organic layers were washed with a saturated aqueous solution of sodium chloride, dried over sodium sulfate, filtered and concentrated under reduced pressure afford the title compound as a yellow oil (624 mg, 68% yield, tᵣ=0.84 min). LCMS (Method F): m/z found 273.3 [M+H]⁺; ¹H-NMR (DMSO-d₆, 400 MHz) δ (ppm) 7.92 (d, J=2.0 Hz, 1H), 7.71 (dd, J=8.5, 2.0 Hz, 1H), 7.64 (s, 1H), 3.73 (s, 2H), 2.45 (q, J=7.6, 7.0 Hz, 4H), 1.68-1.59 (m, 4H).

### Step 3: Synthesis of 1-(3,4-dichlorophenyl)-2-pyrrolidin-1-yl-ethanamine

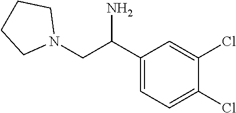 [SMILES: NC(CN1CCCC1)c1ccc(Cl)c(Cl)c1]

In a vial under atmospheric pressure, to a stirred solution of 1-(3,4-dichlorophenyl)-2-pyrrolidin-1-yl-ethanone oxime (620 mg, 2.27 mmol) in anhydrous THF (9.9 mL) at 0° C. under nitrogen was added 2.4 M LAH in THF (2.4 mL, 5.67 mmol). The reaction mixture was stirred at 70° C. for 3 h. The reaction mixture was quenched at 0° C. with water (0.32 mL, same weigh as LAH), 1 M sodium hydroxide (0.23 mL, 0.228 mmol) was added and water (three times the weight of LAH used). The resulting heterogenous mixture was stirred at room temperature for 15 min, dried over sodium sulfate, filtered, rinsed with THF, and concentrated under reduced pressure. The crude was purified by flash chromatography on silica gel using a gradient of ammoniacal methanol in dichloromethane from 1% to 5% to afford the title compound as a yellow oil (296.7 mg, 37% yield, tᵣ=0.49 min). LCMS (Method F): m/z found 259.2 [M+H]⁺; ¹H-NMR (DMSO-d₆, 400 MHz) δ (ppm) 7.64 (d, J=2.0 Hz, 1H), 7.54 (d, J=8.3 Hz, 1H), 7.36 (dd, J=8.3, 2.0 Hz, 1H), 3.97 (dd, J=8.7, 5.4 Hz, 1H), 2.54-2.47 (m, 3H), 2.47-2.40 (m, 2H), 2.36 (dd, J=11.7, 5.4 Hz, 1H), 2.00 (s, 2H), 1.71-1.63 (m, 4H).

### Step 4: Synthesis of N-(1-(3,4-dichlorophenyl)-2-(pyrrolidin-1-yl)ethyl)-4-(trifluoromethoxy)benzenesulfonamide (182)

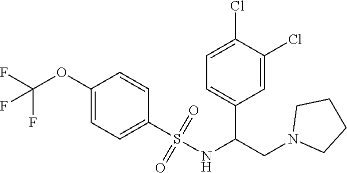 [SMILES: O=S(=O)(NC(CN1CCCC1)c1ccc(Cl)c(Cl)c1)c1ccc(OC(F)(F)F)cc1]

In a sealed tube, 4-(trifluoromethoxy)benzenesulfonyl chloride (43 μL, 0.255 mmol) was added to a stirred solution of 1-(3,4-dichlorophenyl)-2-pyrrolidin-1-yl-ethanamine (60 mg, 0.232 mmol) and triethylamine (97 μL, 0.695 mmol) in anhydrous DCM (2.3358 mL). The solution was stirred at room temperature for 18 h. Water (5 mL) was added and the aqueous layer was extracted with ethyl acetate (1×10 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was triturated in pentane, filtered, washed with pentane and dried under vacuum at 70° C. for 3 days to afford the title compound as a white powder (79.6 mg, 71% yield, 100% purity, tᵣ=1.58 min). LCMS (Method C): m/z found 483.2 [M+H]⁺; ¹H-NMR (DMSO-d₆, 500 MHz): δ (ppm) 8.11-8.62 (m, 1H), 7.73 (d, J=8.8 Hz, 2H), 7.38-7.42 (m, 3H), 7.37 (d, J=2.2 Hz, 1H), 7.17 (dd, J=8.2, 2.1 Hz, 1H), 4.33 (br s, 1H), 2.55-2.68 (m, 1H), 2.45 (br dd, J=12.2, 6.1 Hz, 1H), 2.21-2.42 (m, 4H), 1.54 (br s, 4H).

### Example 65: N-(1-(4-chlorophenyl)-2-(piperidin-1-yl)ethyl)-4-(trifluoromethoxy)benzenesulfonamide (183)

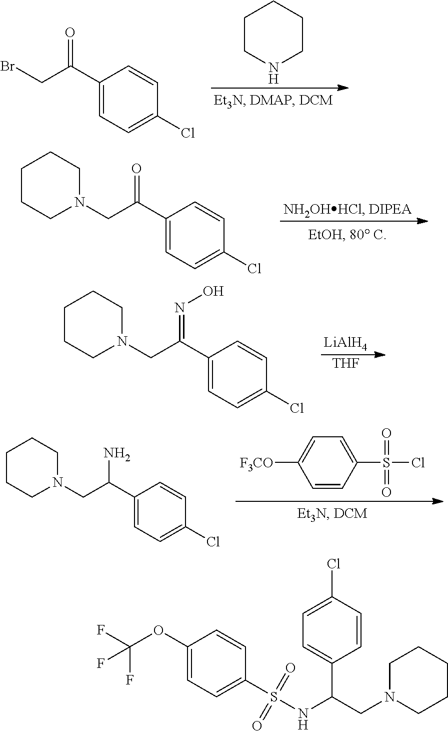

### Step 1: Synthesis of 1-(4-chlorophenyl)-2-(1-piperidyl)ethanone

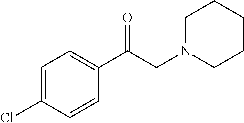 [SMILES: O=C(CN1CCCCC1)c1ccc(Cl)cc1]

In a sealed tube under nitrogen, a solution of 2-bromo-1-(4-chlorophenyl)ethanone (1.00 g, 4.28 mmol), piperidine (761 μL, 7.71 mmol), and DMAP (26 mg, 0.214 mmol) in dry DCM (10 mL) was stirred at 0° C. triethylamine (657 μL, 4.71 mmol) was added and the solution was stirred at rt for 16 h. The mixture was washed with a half saturated aqueous solution of NaHCO₃. The aqueous layer was extracted twice with DCM. The combined organic layers were washed with brine, dried over sodium sulfate, filtered and concentrated. The crude was purified by flash chromatography on silica gel using a gradient of (MeOH+2% NH₄OH) in DCM from 0.4% to 2% to afford the title compound as an orange solid (880 mg, 86% yield, tᵣ=0.50 min). LCMS (Method E): m/z found 238.2 [M+H]⁺; ¹H-NMR (DMSO-d₆, 400 MHz) δ (ppm) 8.07-7.97 (m, 2H), 7.66-7.50 (m, 2H), 3.72 (s, 2H), 2.48-2.37 (m, 4H), 1.48 (p, J=5.6 Hz, 4H), 1.37 (p, J=5.5 Hz, 2H).

### Step 2: Synthesis of 1-(4-chlorophenyl)-2-(1-piperidyl)ethanone oxime

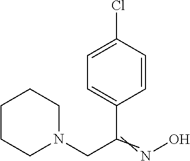 [SMILES: ON=C(CN1CCCCC1)c1ccc(Cl)cc1]

In a sealed tube, DIPEA (1.4 mL, 7.77 mmol) was added to a solution of hydroxylamine hydrochloride (514 mg, 7.40 mmol) and 1-(4-chlorophenyl)-2-(1-piperidyl)ethanone (100%, 880 mg, 3.70 mmol) in ethanol (18 mL). The solution was stirred at 80° C. for 4 h and was concentrated under reduced pressure to afford the title compound as an orange oil (2.13 g, quantitative yield, tᵣ=0.51 min). LCMS (Method F): m/z found 253.2 [M+H]⁺; ¹H-NMR (DMSO-d₆, 400 MHz) δ (ppm) 7.80-7.60 (m, 2H), 7.51-7.38 (m, 2H), 4.35 (t, J=5.0 Hz, 1H), 3.50-3.40 (m, 2H), 2.49-2.37 (m, 3H), 1.51-1.40 (m, 4H), 1.40-1.33 (m, 2H).

### Step 3: Synthesis of 1-(4-chlorophenyl)-2-(1-piperidyl)ethanamine

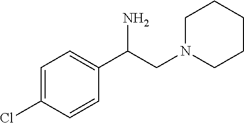 [SMILES: NC(CN1CCCCC1)c1ccc(Cl)cc1]

In a three necked round bottomed flask equipped with a thermometer and a reflux condenser, under nitrogen, a solution of 1-(4-chlorophenyl)-2-(1-piperidyl)ethanone oxime (935 mg, 3.70 mmol) in dry THF (20 mL) was stirred at 0° C. A solution of 2.4 M LAH (3.9 mL, 9.25 mmol) was added dropwise over 2 min to keep the temperature below 10° C. then the mixture was stirred at 70° C. for 2 h. The mixture was allowed to cool to rt then was stirred at 0° C. Water (351 μL) was added dropwise and the mixture was stirred at 0° C. for 15 min. Aqueous NaOH 15% (351 μL) was added dropwise and the mixture was stirred at 0° C. for 30 min. Water (1.05 mL) then EtOAc were added and the mixture was stirred at rt for 30 min. The mixture was filtered through a pad of dicalite, and the residue was thoroughly washed with EtOAc. The filtrate was dried over sodium sulfate, filtered and concentrated. The crude was purified by flash chromatography on silica gel using a gradient of (MeOH+2% NH₄OH) in DCM from 2% to 10% to afford the title compound as a yellow oil (630 mg, 71% yield, tᵣ=0.39 min). LCMS (Method E): m/z found 239.2 [M+H]⁺; ¹H-NMR (DMSO-d₆, 400 MHz) δ (ppm) 7.43-7.37 (m, 2H), 7.37-7.30 (m, 2H), 4.03 (dd, J=9.2, 4.9 Hz, 1H), 2.49-2.40 (m, 2H), 2.33-2.15 (m, 4H), 1.58-1.42 (m, 4H), 1.42-1.32 (m, 2H).

### Step 4: Synthesis of N-(1-(4-chlorophenyl)-2-(piperidin-1-yl)ethyl)-4-(trifluoromethoxy)benzenesulfonamide (183)

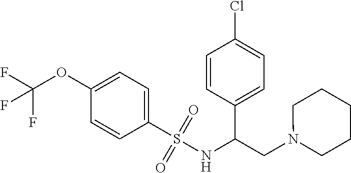 [SMILES: O=S(=O)(NC(CN1CCCCC1)c1ccc(Cl)cc1)c1ccc(OC(F)(F)F)cc1]

In a sealed tube, a solution of 4-(trifluoromethoxy)benzenesulfonyl chloride (50 mg, 0.192 mmol) in DCM (500 μL) was added to a stirred solution of 1-(4-chlorophenyl)-2-(1-piperidyl)ethanamine (50 mg, 0.211 mmol) and triethylamine (107 μL, 0.767 mmol) in DCM (1 mL). The solution was stirred at rt for 18 h and was diluted with half saturated aqueous NaHCO₃. The aqueous layer was extracted with DCM, the combined organic layers were washed with brine, dried over sodium sulfate, filtered and concentrated. The crude was purified by flash chromatography on silica gel using a gradient of (MeOH+2% NH₄OH) in DCM from 0.2% to 5%. The desired fractions were concentrated and triturated in pentane. The obtained suspension was filtered, the residue was washed with pentane and dried under reduced pressure at 45° C. for 16 h to afford the title product as a white powder (62 mg, 69% yield, 100% purity, tᵣ=1.52 min). LCMS (Method C): m/z found 461.3 [M−H]⁻; ¹H-NMR (DMSO-d₆, 500 MHz): δ (ppm) 8.21 (br s, 1H), 7.77 (d, J=7.8 Hz, 2H), 7.42 (d, J=8.1 Hz, 2H), 7.17-7.22 (m, 4H), 4.34 (dd, J=8.8, 5.6 Hz, 1H), 2.44 (dd, J=13.0, 9.0 Hz, 1H), 2.17-2.27 (m, 5H), 1.22-1.37 (m, 6H).

### Example 66: N-(1-(4-chlorophenyl)-2-(4-methylpiperazin-1-yl)ethyl)-4-(trifluoromethoxy)benzenesulfonamide (184)

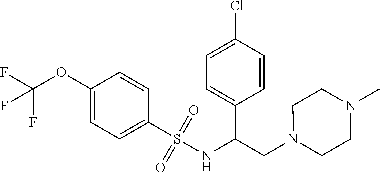 [SMILES: CN1CCN(CC(NS(=O)(=O)c2ccc(OC(F)(F)F)cc2)c2ccc(Cl)cc2)CC1]

In a sealed vial under nitrogen, to a solution of 1-(4-chlorophenyl)-2-(4-methylpiperazin-1-yl)ethanamine (58 mg, 0.229 mmol) and 4-(trifluoromethoxy)benzenesulfonyl chloride (0.039 mL, 0.229 mmol) in anhydrous DCM (2.2855 mL) was added triethylamine (0.13 mL, 0.914 mmol). The solution was stirred at rt overnight, then DCM and an half saturated solution of NaHCO₃ were added and the aqueous layer was extracted with DCM. Organic layers were gathered, filtered through phase separator, concentrated in vacuo and purified by flash chromatography on silica gel using a gradient of MeOH in DCM from 2% to 10%. Desired fractions were gathered, concentrated in vacuo, sonicated in Et₂O and filtered to afford the title compound as a white powder (84 mg, 76% yield, 97.5% purity, tᵣ=1.52 min). LCMS (Method C): m/z found 478.3 [M+H]⁺; ¹H-NMR (DMSO-d₆, 500 MHz): δ (ppm) 8.27 (br d, J=7.6 Hz, 1H), 7.75 (d, J=8.0 Hz, 2H), 7.41 (d, J=8.1 Hz, 2H), 7.17-7.22 (m, 4H), 4.36 (br s, 1H), 2.45-2.49 (m, 1H), 2.21-2.35 (m, 5H), 2.09-2.20 (m, 3H), 2.06-2.08 (m, 1H), 2.07 (s, 3H).

### Example 67: N-(1-(3,4-dichlorophenyl)-2-(4-methylpiperazin-1-yl)ethyl)-4-(trifluoromethoxy)benzenesulfonamide (185)

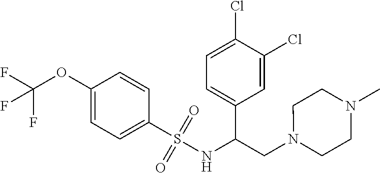 [SMILES: CN1CCN(CC(NS(=O)(=O)c2ccc(OC(F)(F)F)cc2)c2ccc(Cl)c(Cl)c2)CC1]

In a sealed vial under nitrogen, to a solution of 1-(3,4-dichlorophenyl)-2-(4-methylpiperazin-1-yl)ethanamine (75 mg, 0.260 mmol) and 4-(trifluoromethoxy)benzenesulfonyl chloride (0.044 mL, 0.260 mmol) in anhydrous DCM (2.6022 mL) was added triethylamine (0.15 mL, 1.04 mmol). The solution was stirred at rt overnight, then DCM and an half saturated solution of NaHCO₃ were added and the aqueous layer was extracted with DCM. Organic layers were gathered, filtered through phase separator, concentrated in vacuo and purified by flash chromatography on silica gel using a gradient of MeOH in DCM from 2% to 10%. Desired fractions were gathered, concentrated in vacuo, sonicated in Et₂O and filtered to afford the title compound as a white powder (67 mg, 49% yield, 98.6% purity, tᵣ=1.63 min). LCMS (Method C): m/z found 512.2 [M+H]⁺; ¹H-NMR (DMSO-d₆, 500 MHz): δ (ppm) 8.30 (br s, 1H), 7.76 (d, J=7.9 Hz, 2H), 7.40-7.43 (m, 4H), 7.19 (dd, J=8.3, 2.2 Hz, 1H), 4.36-4.45 (m, 1H), 2.38-2.49 (m, 1H), 2.27 (br dd, J=12.8, 5.7 Hz, 5H), 2.07 (s, 3H), 2.03-2.16 (m, 4H).

### Example 68: 4-(N-(1-(3,4-dichlorophenyl)-2-(dimethylamino)ethyl)sulfamoyl)-N-phenylbenzamide (186)

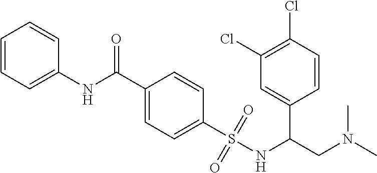 [SMILES: CN(C)CC(NS(=O)(=O)c1ccc(C(=O)Nc2ccccc2)cc1)c1ccc(Cl)c(Cl)c1]

In a round-bottomed flask, to a stirred solution of 4-[[1-(3,4-dichlorophenyl)-2-(dimethylamino)ethyl]sulfamoyl]benzoic acid (88%, 50 mg, 0.105 mmol) in anhydrous DCM (0.6166 mL) at rt were added successively N-ethyl-N-isopropyl-propan-2-amine (55 μL, 0.316 mmol), propanephosphonic acid anhydride (38 μL, 0.158 mmol) and aniline (12 μL, 0.127 mmol). The reaction mixture was stirred at rt overnight. Additional N-ethyl-N-isopropyl-propan-2-amine (37 μL, 0.211 mmol), polypropylphosphonic anhydride (PPPA) (50%, 51 μL, 0.105 mmol), aniline (10 μL, 0.105 mmol) was added and the reaction mixture was stirred at rt for 1.5 h. The organic layer was washed twice with a saturated aqueous solution of NaHCO₃ and once with a saturated aqueous solution of NaCl, dried over sodium sulfate, filtered and concentrated under reduced pressure. The crude was purified by flash chromatography on silica gel (column 12 g, 15 μm) using a gradient of EtOH in DCM from 0% to 5% and the desired fractions were combined and concentrated. The resulting oil was solubilized in DCM and the homogenous solution was added to pentane. The precipitate formed was filtered, washed with pentane and dried under vacuum at 40° C. for 48 h to afford the title compound as a white solid (52 mg, 99.524% yield, 99.4% purity, tᵣ=1.33 min). LCMS (Method C): m/z found 492.3 [M+H]⁺; ¹H-NMR (DMSO-d₆, 600 MHz): δ (ppm) 10.30 (s, 1H), 8.30 (br dd, J=3.4, 2.4 Hz, 1H), 8.01 (d, J=8.5 Hz, 2H), 7.72-7.84 (m, 4H), 7.43 (d, J=8.4 Hz, 1H), 7.41 (d, J=2.1 Hz, 1H), 7.37 (dd, J=8.4, 7.5 Hz, 2H), 7.19 (dd, J=8.2, 2.1 Hz, 1H), 7.13 (t, J=7.3 Hz, 1H), 4.38 (br s, 1H), 2.27-2.45 (m, 2H), 2.04 (s, 6H).

### Example 69: N-cyclohexyl-4-(N-(1-(3,4-dichlorophenyl)-2-(dimethylamino)ethyl)sulfamoyl)benzamide (187)

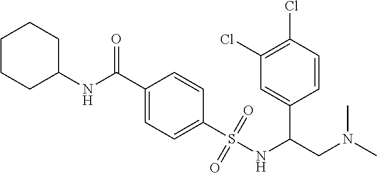 [SMILES: CN(C)CC(NS(=O)(=O)c1ccc(C(=O)NC2CCCCC2)cc1)c1ccc(Cl)c(Cl)c1]

In a round-bottomed flask, to a stirred suspension of 4-[[1-(3,4-dichlorophenyl)-2-(dimethylamino)ethyl]sulfamoyl]benzoic acid (88%, 100 mg, 0.211 mmol) in anhydrous DCM (1.5 mL) at rt were added successively N-ethyl-N-isopropyl-propan-2-amine (110 μL, 0.630 mmol), 2,4,6-tripropyl-1,3,5,2,4,6-trioxatriphosphinane 2,4,6-trioxide (78 μL, 0.323 mmol) and cyclohexanamine (29 μL, 0.252 mmol). The reaction mixture was stirred at rt under nitrogen for 4 h. Additional N-ethyl-N-isopropyl-propan-2-amine (18 μL, 0.105 mmol), cyclohexanamine (4.8 μL, 0.0420 mmol) and 2,4,6-tripropyl-1,3,5,2,4,6-trioxatriphosphinane 2,4,6-trioxide (25 μL, 0.211 mmol) were added at rt and the reaction mixture was stirred under nitrogen at rt for 2 h. The reaction mixture was diluted with DCM. The organic layer was washed twice with a saturated aqueous solution of NaHCO₃ and once with a saturated aqueous solution of NaCl, dried using a phase separator and concentrated under reduced pressure. The crude was purified by flash chromatography on silica gel using a gradient of EtOH in DCM from 0.5% to 5%. The desired fraction was combined and concentrated to afford the title compound. The resulting gum was solubilized in a minimum of DCM and the homogenous solution was added to heptane. The precipitate formed was filtered, washed with heptane and dried under vacuum at 40° C. for two days to afford the title compound as a white powder (53 mg, 50.421% yield, 98.2% purity, tᵣ=1.39 min). LCMS (Method C): m s found 498.3 [M+H]⁺; ¹H-NMR (500 MHz, DMSO-d₆) δ (ppm) 8.31-8.26 (m, 1H), 8.25-8.16 (m, 1H), 7.87 (d, J=8.6 Hz, 2H), 7.68 (d, J=8.6 Hz, 2H), 7.40 (d, J=8.1 Hz, 1H), 7.36 (d, J=2.0 Hz, 1H), 7.18-7.12 (m, 1H), 4.48-4.20 (m, 1H), 3.88-3.60 (m, 1H), 2.43-2.25 (m, 2H), 2.02 (s, 6H), 1.88-1.69 (m, 4H), 1.66-1.56 (m, 1H), 1.31 (br s, 5H).

### Example 70: N-(1-(3,4-dichlorophenyl)-2-(dimethylamino)ethyl)-4-(phenylamino)benzenesulfonamide (188)

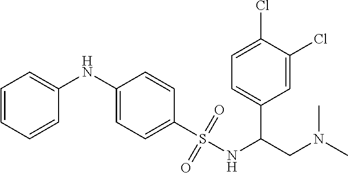 [SMILES: CN(C)CC(NS(=O)(=O)c1ccc(Nc2ccccc2)cc1)c1ccc(Cl)c(Cl)c1]

A sealed vial was charged with 4-bromo-N-[1-(3,4-dichlorophenyl)-2-(dimethylamino)ethyl]benzenesulfonamide (62 mg, 0.137 mmol), [1-(2-diphenylphosphanyl-1-naphthyl)-2-naphthyl]-diphenyl-phosphane (13 mg, 0.0206 mmol), cesium carbonate (134 mg, 0.411 mmol) and aniline (25 μL, 0.274 mmol) in anhydrous 1,4-dioxane (2 mL). The reaction mixture was degassed with Ar for 15 min. Then aniline (25 μL, 0.274 mmol) was added and the reaction mixture was heated at 100° C. overnight. Additional [1-(2-diphenylphosphanyl-1-naphthyl)-2-naphthyl]-diphenyl-phosphane (13 mg, 0.0206 mmol), cesium carbonate (134 mg, 0.411 mmol), aniline (25 μL, 0.274 mmol) and Pd(OAc)₂ (3.1 mg, 0.0137 mmol) was added at rt under Ar and the reaction mixture was stirred at 100° C. overnight. The reaction mixture was filtered through talcum powder and the filtrate was washed once with water, once with a saturated aqueous solution of NH₄Cl and once with a saturated aqueous solution of NaCl, dried over sodium sulfate, filtered and concentrated under reduced pressure. The crude was purified by flash chromatography on silica gel using a gradient of MeOH in DCM from 0.2% to 5%. The desired fractions were combined and concentrated. The residue was re-purified by reverse-phase preparative chromatography (C18-AQ 50 g) using a gradient of acetonitrile in water from 0% to 100% (0.1% AcOH in both solvents). The desired fractions were combined and concentrated. The residue was triturated in pentane, filtered, washed with pentane and dried under vacuum at 45° C. for 20 h to afford the title compound as a white powder (42.6 mg). The resulting batch was dried again at 70° C. under vacuum to remove residual AcOH (0.71 mmol) to afford a beige solid (35 mg). Remaining residual AcOH was removed by drying under vacuum at 60° C. for two days to afford a beige solid (34.2 mg). The resulting product was dissolved in diethyl ether and then 200 μL of 2 N HCl in diethyl ether was added. The resulting suspension was filtered, washed twice with diethyl ether and once with pentane. The powder was then dried under vacuum at 45° C. for 20 h to afford a white powder (4 mg, 42% yield, 98.9% purity, tᵣ=1.45 min). LCMS (Method C): m/z found 464.2 [M+H]⁺; ¹H-NMR (DMSO-d₆, 500 MHz): δ (ppm) 9.60 (br s, 1H), 8.67 (s, 1H), 8.40 (br d, J=8.6 Hz, 1H), 7.47 (d, J=2.0 Hz, 1H), 7.45 (d, J=8.3 Hz, 1H), 7.29-7.34 (m, 4H), 7.26 (dd, J=8.3, 2.0 Hz, 1H), 7.07 (d, J=7.6 Hz, 2H), 7.00 (t, J=7.5 Hz, 1H), 6.79 (d, J=8.8 Hz, 2H), 4.67-4.83 (m, 1H), 3.32-3.36 (m, 1H), 3.14-3.25 (m, 1H), 2.68-3.00 (m, 6H).

### Example 71: N-(2-(dimethylamino)-1-(3-(trifluoromethyl)phenyl)ethyl)-4-phenoxybenzenesulfonamide (189)

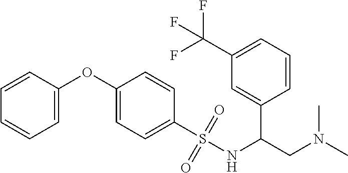 [SMILES: CN(C)CC(NS(=O)(=O)c1ccc(Oc2ccccc2)cc1)c1cccc(C(F)(F)F)c1]

In a sealed tube, 4-phenoxybenzenesulphonyl chloride (55 mg, 0.205 mmol) was added to a stirred solution of N′,N′-dimethyl-1-[3-(trifluoromethyl)phenyl]ethane-1,2-diamine (95%, 50 mg, 0.205 mmol) and triethylamine (86 μL, 0.614 mmol) in anhydrous DCM (2.0683 mL). The solution was stirred at room temperature for 18 h. Water (5 mL) was added and the aqueous layer was extracted with dichloromethane (1×10 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was triturated in 2 M hydrogen chloride in diethyl ether (1.0 mL, 2.05 mmol), filtered, washed with diethyl ether and dried under vacuum at 50° C. for 3 days to afford the title compound as a white powder (59.2 mg, 58% yield, 99.5% purity, tᵣ=1.6 min). LCMS (Method C): m/z found 465 [M+H]⁺; ¹H-NMR (DMSO-d₆, 500 MHz): δ (ppm) 9.67 (br s, 1H), 8.59-8.88 (m, 1H), 7.62 (s, 1H), 7.57 (t, J=8.3 Hz, 2H), 7.44-7.52 (m, 4H), 7.39-7.44 (m, 1H), 7.20-7.28 (m, 1H), 6.92 (d, J=7.6 Hz, 2H), 6.73 (d, J=8.8 Hz, 2H), 4.93 (br s, 1H), 3.16-3.44 (m, 2H), 2.74-3.02 (m, 6H).

### Example 72: N-(1-(4-chlorophenyl)-2-(dimethylamino)ethyl)-4-phenoxybenzenesulfonamide (190)

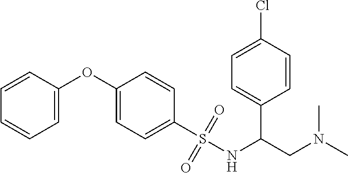 [SMILES: CN(C)CC(NS(=O)(=O)c1ccc(Oc2ccccc2)cc1)c1ccc(Cl)cc1]

In a sealed tube, 4-phenoxybenzenesulphonyl chloride (64 mg, 0.239 mmol) was added to a stirred solution of 1-(4-chlorophenyl)-N′,N′-dimethyl-ethane-1,2-diamine (95%, 50 mg, 0.239 mmol) and triethylamine (100 μL, 0.717 mmol) in anhydrous DCM (2.4 mL). The solution was stirred at room temperature for 18 h. Water (5 mL) was added and the aqueous layer was extracted with dichloromethane (1×10 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was triturated in pentane, filtered, washed with pentane and dried under vacuum at 50° C. for 3 days to afford the title compound as a white powder (60.3 mg, 59% yield, 99.9% purity, tᵣ=1.53 min). LCMS (Method C): m/z found 431 [M+H]⁺; ¹H-NMR (DMSO-d₆, 500 MHz): δ (ppm) 8.05 (br d, J=7.8 Hz, 1H), 7.55-7.58 (m, 2H), 7.47 (t, J=7.3 Hz, 2H), 7.20-7.26 (m, 3H), 7.14-7.17 (m, 2H), 7.03 (d, J=7.6 Hz, 2H), 6.89-6.92 (m, 2H), 4.29 (q, J=7.5 Hz, 1H), 2.35-2.44 (m, 1H), 2.18-2.32 (m, 1H), 2.06 (s, 6H).

### Example 73: 3-chloro-N-(2-(dimethylamino)-1-(3-(trifluoromethyl)phenyl)ethyl)-4-(trifluoromethoxy)benzenesulfonamide (191)

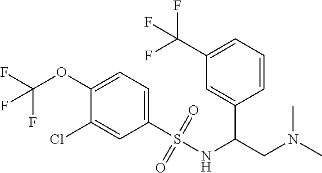

In a sealed tube, 3-chloro-4-(trifluoromethoxy)benzenesulfonyl chloride (98%, 62 mg, 0.205 mmol) was added to a stirred solution of N′,N′-dimethyl-1-[3-(trifluoromethyl)phenyl]ethane-1,2-diamine (95%, 50 mg, 0.205 mmol) and triethylamine (86 μL, 0.614 mmol) in Anhydrous DCM (2.0683 mL). The solution was stirred at room temperature for 18 h. Water (5 mL) was added and the aqueous layer was extracted with dichloromethane (1×10 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was triturated in 2 M hydrogen chloride in diethyl ether (1.0 mL, 2.05 mmol), filtered, washed with diethyl ether and dried under vacuum at 50° C. for 3 days to afford the title compound as a white powder (67.9 mg, 63% yield, 99.4% purity, tᵣ=1.61 min). LCMS (Method C): m/z 491 [M+H]⁺; 1H-NMR (DMSO-d₆, 500 MHz): δ (ppm) 9.70 (br s, 1H), 9.12 (d, J=9.8 Hz, 1H), 7.67 (d, J=2.2 Hz, 1H), 7.57-7.63 (m, 3H), 7.46 (br d, J=5.6 Hz, 2H), 7.35-7.39 (m, 1H), 4.89-5.11 (m, 1H), 3.37-3.51 (m, 1H), 3.22 (br s, 1H), 2.94 (br s, 3H), 2.86 (br s, 3H).

### Example 74: 3-chloro-N-(1-(4-chlorophenyl)-2-(dimethylamino)ethyl)-4-(trifluoromethoxy)benzenesulfonamide (192)

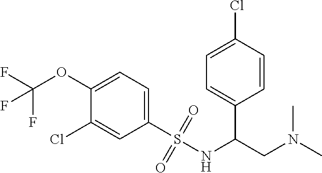

In a sealed tube, 3-chloro-4-(trifluoromethoxy)benzenesulfonyl chloride (98%, 72 mg, 0.239 mmol) was added to a stirred solution of 1-(4-chlorophenyl)-N′,N′-dimethyl-ethane-1,2-diamine (95%, 50 mg, 0.239 mmol) and triethylamine (100 μL, 0.717 mmol) in anhydrous DCM (2.4 mL). The solution was stirred at room temperature for 18 h. Water (5 mL) was added and the aqueous layer was extracted with dichloromethane (1×10 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was triturated in 2 M hydrogen chloride in diethyl ether (1.2 mL, 2.39 mmol), filtered, washed with diethyl ether and dried under vacuum at 50° C. for 3 days to afford the title compound as a white powder (92.1 mg, 76.9% yield, 98.6% purity, tᵣ=1.54 min). LCMS (Method C): m/z found 457 [M+H]⁺; ¹H-NMR (DMSO-d₆, 500 MHz): δ (ppm) 9.69 (br s, 1H), 8.87-9.24 (m, 1H), 7.64 (d, J=2.2 Hz, 1H), 7.58-7.63 (m, 1H), 7.52 (dd, J=8.7, 1.3 Hz, 1H), 7.24 (d, J=8.6 Hz, 2H), 7.15 (d, J=8.6 Hz, 2H), 4.86 (br s, 1H), 3.37-3.51 (m, 1H), 3.13-3.27 (m, 1H), 2.87 (br d, J=14.2 Hz, 6H).

### Example 75: N-(2-(dimethylamino)-1-(3-(trifluoromethyl)phenyl)ethyl)-4-(4-(trifluoromethyl)phenoxy)benzenesulfonamide (193)

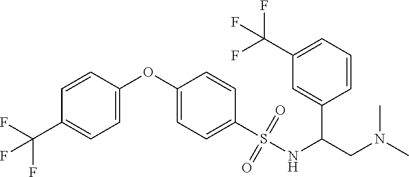 [SMILES: CN(C)CC(NS(=O)(=O)c1ccc(Oc2ccc(C(F)(F)F)cc2)cc1)c1cccc(C(F)(F)F)c1]

In a sealed tube, 4-[4-(trifluoromethyl)phenoxy]benzenesulfonyl chloride (95%, 72 mg, 0.205 mmol) was added to a stirred solution of N′,N′-dimethyl-1-[3-(trifluoromethyl)phenyl]ethane-1,2-diamine (95%, 50 mg, 0.205 mmol) and triethylamine (86 μL, 0.614 mmol) in anhydrous DCM (2 mL). The solution was stirred at room temperature for 4 h. Water (5 mL) was added and the aqueous layer was extracted with dichloromethane (1×8 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was triturated in 2 M hydrogen chloride in diethyl ether (1.0 mL, 2.05 mmol), filtered, washed with diethyl ether and dried under vacuum at 50° C. for 21 h to afford the title compound as a white powder (88.3 mg, 76% yield, 99.7% purity, tᵣ=1.84 min). LCMS (Method C): m/z found 533.3 [M+H]⁺; ¹H-NMR (DMSO-d₆, 600 MHz): δ (ppm) 9.72 (br s, 1H), 8.87 (d, J=9.1 Hz, 1H), 7.82 (d, J=8.7 Hz, 2H), 7.65 (s, 1H), 7.61 (d, J=7.8 Hz, 1H), 7.58 (br d, J=7.8 Hz, 1H), 7.53-7.56 (m, 2H), 7.41-7.45 (m, 1H), 7.05 (d, J=8.5 Hz, 2H), 6.88 (d, J=8.8 Hz, 2H), 4.96 (br t, J=10.3 Hz, 1H), 3.34-3.43 (m, 1H), 3.19-3.27 (m, 1H), 2.92 (br s, 3H), 2.85 (br s, 3H).

### Example 76: N-(1-(4-chlorophenyl)-2-(dimethylamino)ethyl)-4-(4-(trifluoromethyl)phenoxy)benzenesulfonamide (194)

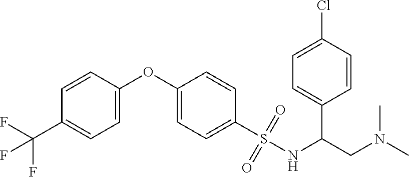 [SMILES: CN(C)CC(NS(=O)(=O)c1ccc(Oc2ccc(C(F)(F)F)cc2)cc1)c1ccc(Cl)cc1]

In a sealed tube, to a stirred solution of triethylamine (95 μL, 0.683 mmol) in anhydrous DCM (2.0265 mL) at rt under nitrogen were added successively 4-[4-(trifluoromethyl)phenoxy]benzenesulfonyl chloride (80 mg, 0.237 mmol), and 1-(4-chlorophenyl)-N′,N′-dimethyl-ethane-1,2-diamine (95%, 47 mg, 0.226 mmol). The solution was stirred at rt for 5 h and was diluted with water. The aqueous layer was extracted twice with DCM, the combined organic layers were washed with a saturated aqueous solution of NH₄Cl, a saturated aqueous solution of NaHCO₃ and a saturated aqueous solution of NaCl, dried using a phase separator and concentrated. The crude was purified by flash chromatography on silica gel using a gradient of MeOH in DCM from 0.2% to 5%. The resulting oil was solubilized in Et₂O and the homogenous solution was added to 2 M hydrogen chloride (1.1 mL, 2.26 mmol) in Et₂O. The resulting suspension was stirred at rt for 2 h. The precipitate formed was filtered, washed with Et₂O and dried under vacuum at 45° C. overnight to afford the title compound as a white powder (65.7 mg, 54.37% yield, 100% purity, tᵣ=1.93 min). LCMS (Method C): m/z found 499 [M+H]⁺; ¹H-NMR (DMSO-d₆, 500 MHz): δ (ppm) 9.61 (br s, 1H), 8.66-8.82 (m, 1H), 7.83 (d, J=8.6 Hz, 2H), 7.56 (d, J=8.8 Hz, 2H), 7.21-7.31 (m, 4H), 7.10 (d, J=8.6 Hz, 2H), 6.97 (d, J=8.8 Hz, 2H), 4.74-4.91 (m, 1H), 3.33-3.42 (m, 1H), 3.12-3.26 (m, 1H), 2.77-3.04 (m, 6H).

### Example 77: N-(1-(3,4-dichlorophenyl)-2-(dimethylamino)ethyl)-[1,1′-biphenyl]-4-sulfonamide (195 and 196

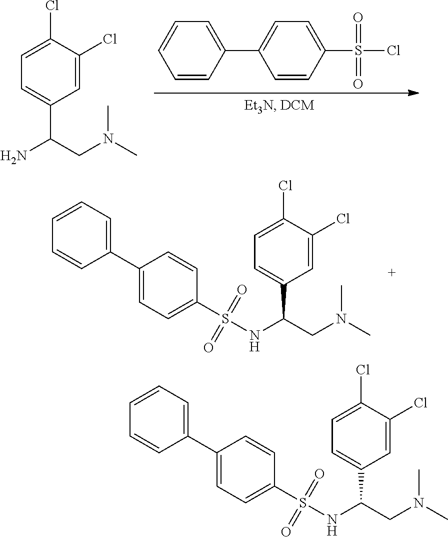

In a sealed tube, biphenyl-4-sulfonyl chloride (97%, 559 mg, 2.14 mmol) was added to a stirred solution of 1-(3,4-dichlorophenyl)-N′,N′-dimethyl-ethane-1,2-diamine (100%, 500 mg, 2.14 mmol) and triethylamine (897 μL, 6.43 mmol) in anhydrous DCM (22 mL). The solution was stirred at room temperature for 20 h. Water (5 mL) was added and the aqueous layer was extracted with dichloromethane (1×5 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was subjected to chiral separation two afford two batches of separated enantiomers. Each batch was purified by flash chromatography on silica gel using a gradient of methanol in dichloromethane from 0% to 5%. The residues were triturated in pentane, filtered, washed with pentane and dried under vacuum at 70° C. for 18 h to afford a the racemic title compound as a white powder. 820 mg of the racemic mixture was dissolved by sonication in 19 mL (EtOH+0.5% i-PrNH₂) and 3 mL (ACN), with heating, to provide a solution having a concentration of 37.3 mg/mL. The solution was passed through a GHP filter (0.45 μm). 110 injections of the sample solution were performed on a Chiralsel OJ-H column (250×21 mm, 5 μm) using 17% (EtOH+0.5% i-PrNH₂) in CO₂ as a mobile phase (Waters Prep SFC200, flow rate=50 mL/min, T=40° C., P=150 bar, UV detection at 210 nm) to afford the separated enantiomers.

Enantiomer I (195): (338.7 mg, 35% yield, 99.8% purity, tᵣ=1.77 min). LCMS (Method C): m/z found 449 [M+H]⁺; ¹H-NMR (500 MHz, DMSO-d₆) δ (ppm) 8.17 (m, J=6.36 Hz, 1H), 7.62-7.66 (m, 6H), 7.50 (t, J=7.26 Hz, 2H), 7.33-7.45 (m, 3H), 7.16 (dd, J=1.96, 8.31 Hz, 1H), 4.36 (m, 1H), 2.42 (dd, J=8.07, 12.47 Hz, 1H), 2.32 (dd, J=7.09, 12.47 Hz, 1H), 2.06 (s, 6H).

Enantiomer II (196): (218.7 mg, 23% yield, 99.6% purity, tᵣ=1.77 min). LCMS (Method C): m/z found 449 [M+H]⁺; ¹H-NMR (500 MHz, DMSO-d₆) δ (ppm) 8.17 (br s, 1H), 7.61-7.67 (m, 6H), 7.50 (t, J=7.00 Hz, 2H), 7.40-7.45 (m, 1H), 7.35-7.39 (m, 1H), 7.31-7.34 (m, 1H), 7.16 (d, J=8.11 Hz, 1H), 4.36 (br t, J=7.09 Hz, 1H), 2.42 (dd, J=8.07, 12.47 Hz, 1H), 2.32 (dd, J=7.09, 12.47 Hz, 1H), 2.06 (s, 6H).

### Example 78: N-(1-(3,4-dichlorophenyl)-2-(dimethylamino)ethyl)-[1,1′-biphenyl]-3-sulfonamide (197 and 198

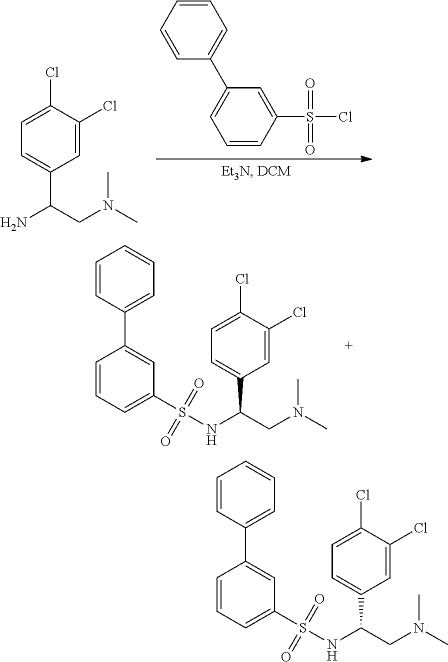

In a sealed tube, biphenyl-3-sulfonyl chloride (95%, 203 mg, 0.763 mmol) was added to a stirred solution of 1-(3,4-dichlorophenyl)-N²,N²-dimethylethane-1,2-diamine (95%, 187 mg, 0.763 mmol) and triethylamine (0.32 mL, 2.29 mmol) in anhydrous DCM (8.2401 mL). The solution was stirred at room temperature overnight. Water (2 mL) was added and the aqueous layer was extracted with dichloromethane (1×2 mL). The combined organic layers were washed with a saturated aqueous solution of sodium chloride, dried over sodium sulfate, filtered and concentrated under reduced pressure to afford a yellow oil (437 mg). The crude was purified by flash chromatography on silica gel (column 25 g, 50 μm) using a gradient of methanol in dichloromethane from 0% to 8%. The desired fractions were combined and concentrated to afford a yellow foam (296 mg). The residue was subjected to chiral separation to afford two batches of separated enantiomers. Each batch was dissolved in Et₂O and a solution of 2 N HCl in Et₂O was added. The 2 mixtures were stirred for 15 min and the resulting suspension was filtered, washed with Et₂O and pentane. The resulting solid dried under vacuum at 50° C. for 18 h to afford the racemic title compounds as a white solid. 267 mg of the racemic mixture was dissolved by sonication in MeOH (3 mL), EtOH (3 mL), and a mixture of heptane/EtOH (70:30), to provide a solution having a concentration of 6.5 mg/mL. The solution was passed through a GHP filter (0.45 μm). 1 injection of the sample solution were performed on a Chiralpak AS column (300×76 mm, 20 μm) using 30% EtOH in heptane (Novasep Lab LC, flow rate=275 mL/min, UV detection at 254 nm) to afford separated enantiomers (pure Enantiomer II). 157 mg of a mixture comprising the first enantiomer was dissolved by sonication in 10 mL of ACN and 5 mL of 10 mM aq. NH₄OAc to provide a solution having a concentration of 10.5 mg/mL. 1 injection of sample solution was performed on a Kromasil C18 column (300×50 mm, 10 μm) using a gradient of (ACN/H₂O (9:1)+10 mM NH₄OAc) in 10 mM aq. NH₄OAc from 65% to 100% as a mobile phase (Waters Preparative HPLC LC150, flow rate=120 mL/min, UV detection at 220 nm) to afford the separated Enantiomer I.

Enantiomer I (197): (38.9 mg, 10.492% yield, 100% purity, tᵣ=1.59 min). LCMS (Method C): m/z found 449.2 [M+H]⁺; ¹H-NMR (500 MHz, DMSO-d₆) δ (ppm) 9.68 (br s, 1H), 8.83 (br d, J=9.8 Hz, 1H), 7.76-7.64 (m, 2H), 7.61-7.53 (m, 1H), 7.52-7.37 (m, 7H), 7.26-7.22 (m, 1H), 7.21-7.17 (m, 1H), 4.90 (td, J=10.3, 3.5 Hz, 1H), 3.54-3.11 (m, 2H), 3.01-2.75 (m, 6H).

Enantiomer II (198): (108.6 mg, 29.262% yield, 99.9% purity, tᵣ=1.59 min). LCMS (Method C): m/z found 449.3 [M+H]⁺; ¹H-NMR (500 MHz, DMSO-d₆) δ (ppm) 9.69 (br s, 1H), 8.83 (br d, J=10.0 Hz, 1H), 7.77-7.65 (m, 2H), 7.57 (dt, J=7.8, 1.3 Hz, 1H), 7.52-7.37 (m, 7H), 7.27-7.22 (m, 1H), 7.22-7.18 (m, 1H), 4.96-4.84 (m, 1H), 3.51-3.07 (m, 2H), 3.01-2.72 (m, 6H).

### Example 79: N-(1-(3,4-dichlorophenyl)-2-(dimethylamino)ethyl)-4-propylbenzenesulfonamide (199 and 200

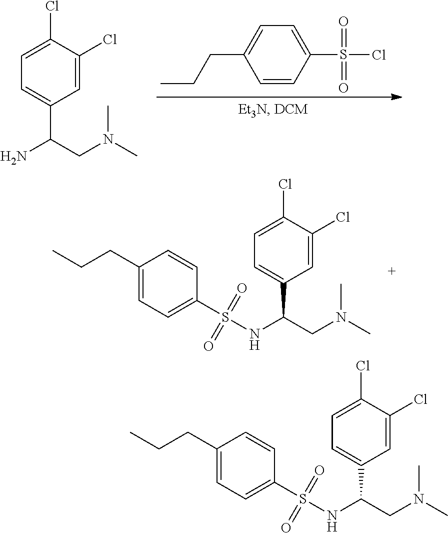

In a sealed tube, 4-propylbenzenesulfonyl chloride (364 μL, 2.04 mmol) was added to a stirred solution of 1-(3,4-dichlorophenyl)-N²,N²-dimethylethane-1,2-diamine (95%, 500 mg, 2.04 mmol) and triethylamine (852 μL, 6.11 mmol) in anhydrous DCM (10.187 mL). The solution was stirred at room temperature for 20 h. Water was added and the aqueous layer was extracted twice with dichloromethane. The combined organic layers were washed with an half saturated aqueous solution of NaHCO₃ dried over a phase separator and concentrated under reduced pressure. The crude was purified by flash chromatography on silica gel using a gradient of methanol (0.7 N NH₃) in dichloromethane from 0.4% to 2% to afford the racemic product as a yellow oil (776.5 mg, 92% yield). The batch was subjected to chiral separation. 764 mg of the racemic mixture was dissolved by sonication in 21 mL (MeOH+0.5% i-PrNH₂) and 2 mL (ACN) to provide a solution having a concentration of 33.2 mg/mL. The solution was passed through a GHP filter (0.45 μm). 115 injections of the sample solution were performed on a Chiralpak IG column (250×30 mm, 5 μm) using 30% (MeOH+0.5% i-PrNH₂) in CO₂ as a mobile phase (Waters Prep SFC200, flow rate=100 mL/min, T=40° C., P=150 bar, UV detection at 210 nm) to afford the separated enantiomers.

Enantiomer I (199): 347 mg of compound 199 was obtained as a colorless oil, which was dissolved in DCM and methanol, 10 mL hydrogen chloride 2 M in diethyl ether was added and the mixture was stirred overnight at RT an concentrated in vacuo, triturated in diethyl ether to afford the title compound as a white solid (345.6 mg, 37% yield, 99.5% purity, tᵣ=1.76 min). LCMS (Method C): m/z found 415 [M+H]⁺; ¹H-NMR (500 MHz, DMSO-d₆) δ (ppm) 9.73 (br s, 1H), 8.70 (br d, J=10.0 Hz, 1H), 7.45-7.41 (m, 3H), 7.34 (d, J=8.3 Hz, 1H), 7.21 (dd, J=8.4, 2.1 Hz, 1H), 7.10 (d, J=8.3 Hz, 2H), 4.81 (td, J=10.5, 3.7 Hz, 1H), 3.40-3.33 (m, 1H), 3.25-3.14 (m, 1H), 2.85 (br d, J=18.8 Hz, 6H), 2.50-2.50 (m, 2H), 1.50 (sxt, J=7.4 Hz, 2H), 0.84 (t, J=7.3 Hz, 3H).

Enantiomer II (200): 356 mg compound 200 was obtained as a colorless oil which was dissolved in DCM and methanol, 10 mL hydrogen chloride 2 M in diethyl ether was added and the mixture was stirred overnight at RT an concentrated in vacuo, triturated in diethyl ether to afford the title compound as a white solid (358.7 mg, 38% yield, 99.2% purity, tᵣ=1.78 min). LCMS (Method C): m/z found 415 [M+H]⁺; ¹H-NMR (500 MHz, DMSO-d₆) δ (ppm) 9.73 (br s, 1H), 8.70 (br d, J=10.0 Hz, 1H), 7.45-7.41 (m, 3H), 7.34 (d, J=8.3 Hz, 1H), 7.21 (dd, J=8.4, 2.1 Hz, 1H), 7.10 (d, J=8.3 Hz, 2H), 4.81 (td, J=10.5, 3.7 Hz, 1H), 3.40-3.33 (m, 1H), 3.25-3.14 (m, 1H), 2.85 (br d, J=18.8 Hz, 6H), 2.50-2.50 (m, 2H), 1.50 (sxt, J=7.4 Hz, 2H), 0.84 (t, J=7.3 Hz, 3H).

### Example 80: 3-chloro-N-(1-(3,4-dichlorophenyl)-2-(dimethylamino)ethyl)-4-(trifluoromethoxy)benzenesulfonamide (201 and 202

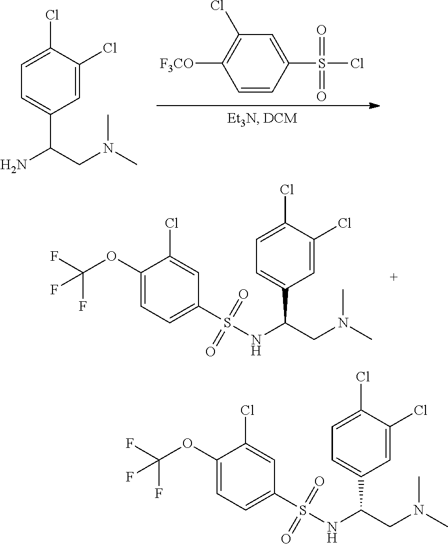

In a sealed tube, 3-chloro-4-(trifluoromethoxy)benzenesulfonyl chloride (98%, 646 mg, 2.14 mmol) was added to a stirred solution of 1-(3,4-dichlorophenyl)-N′,N′-dimethyl-ethane-1,2-diamine (100%, 500 mg, 2.14 mmol) and triethylamine (897 μL, 6.43 mmol) in anhydrous DCM (22 mL). The solution was stirred at room temperature for 3 h. The reaction mixture was diluted with DCM. The organic layer was washed once with a saturated aqueous solution of NH₄Cl, once with a saturated solution of NaHCO₃, and once with a saturated aqueous solution of sodium chloride. The organic layer was passed through a phase separator and concentrated under reduced pressure. The crude was purified by flash chromatography on silica gel using a gradient of methanol in dichloromethane from 0.2% to 5%. The residue was subjected to chiral separation. 920 mg of the racemic mixture was dissolved by sonication in 23 mL (EtOH+0.5% i-PrNH₂) to provide a solution having a concentration of 40.0 mg/mL. The solution was passed through a GHP filter (0.45 μm). 46 injections of the sample solution were performed on a Chiralpak AS column (250×20 mm, 5 μm) using 9% (EtOH+0.5% i-PrNH₂) in CO₂ as a mobile phase (Waters Prep SFC200, flow rate=50 mL/min, T=40° C., P=130 bar, UV detection at 210 nm) to afford the separated enantiomers. The separated enantiomers were dried under vacuum at 45° C. for 18 h to afford the title compounds each as a white powder.

Enantiomer I (201): (375.6 mg, 36% yield, 99.2% purity, tᵣ=1.62 min). LCMS (Method C): m/z found 491.1 [M+H]⁺; ¹H-NMR (500 MHz, DMSO-d₆) δ (ppm) 8.49 (br s, 1H), 7.74 (d, J=1.96 Hz, 1H), 7.66 (dd, J=2.20, 8.56 Hz, 1H), 7.60 (d, J=8.46 Hz, 1H), 7.37-7.40 (m, 2H), 7.16 (dd, J=2.20, 8.31 Hz, 1H), 4.42 (dd, J=6.48, 8.44 Hz, 1H), 2.44-2.48 (m, 1H), 2.28 (dd, J=6.36, 12.72 Hz, 1H), 2.07 (s, 6H).

Enantiomer II (202): (365.6 mg, 35% yield, 98.7% purity, tᵣ=1.62 min). LCMS (Method C): m/z found 491.1 [M+H]⁺; ¹H-NMR (500 MHz, DMSO-d₆) δ (ppm) 8.50 (br s, 1H), 7.74 (d, J=2.20 Hz, 1H), 7.66 (dd, J=2.20, 8.56 Hz, 1H), 7.60 (d, J=8.50 Hz, 1H), 7.37-7.40 (m, 2H), 7.16 (dd, J=1.96, 8.31 Hz, 1H), 4.42 (dd, J=6.48, 8.44 Hz, 1H), 2.44-2.48 (m, 1H), 2.28 (dd, J=6.36, 12.72 Hz, 1H), 2.07 (s, 6H).

### Example 81: N-(1-(3,4-dichlorophenyl)-2-(dimethylamino)ethyl)-3-nitro-4-(trifluoromethoxy)benzenesulfonamide (203 and 204

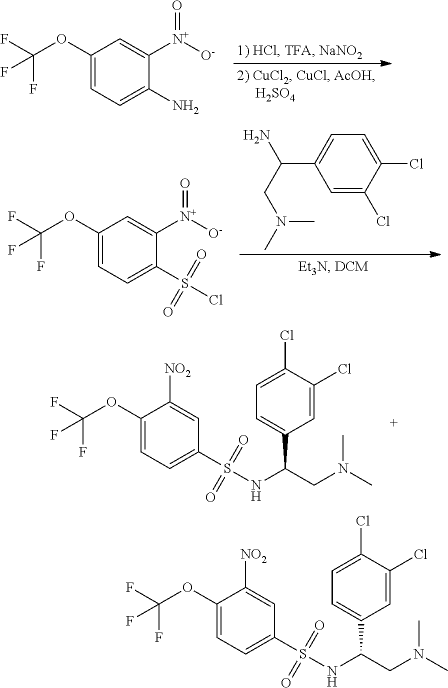

### Step 1: Synthesis of 3-nitro-4-(trifluoromethoxy)benzenesulfonyl chloride

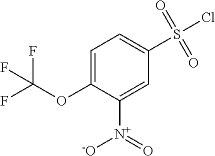

In a three necked round bottomed flask equipped with a thermometer, a solution of 3-nitro-4-(trifluoromethoxy)aniline (98%, 1.50 g, 6.62 mmol) in TFA (22.5 mL) was stirred at 0° C. Copper(I) chloride (20 mg, 0.199 mmol) was added, the mixture was stirred at 0° C. for 5 min, then a solution of sodium nitrite (579 mg, 8.27 mmol) in water (2.4 mL) was added dropwise over 20 min to keep the temperature below 0° C. The mixture was stirred at −5° C. for 30 min to afford Solution A. In a three necked round bottomed flask equipped with a thermometer, a solution of copper(II) dichloride (454 mg, 3.38 mmol) and concentrated hydrogen chloride (2.2 mL, 72.8 mmol) in acetic acid (22.5 mL) and sulfurous acid (23 mL, 0.291 mol) was stirred at −5° C. Solution A was added dropwise over 20 min to keep the temperature below 5° C. The mixture was stirred at 0° C. for 5 min then a rt for 1 h and was extracted twice with DCM. The combined organic layers were dried over sodium sulfate, filtered and concentrated to afford the title compound as a brown oil (1.82 g, 87% yield, tᵣ=0.78 min). LCMS (Method F); ¹H-NMR (DMSO-d₆, 400 MHz) δ (ppm) 8.24 (d, J=2.1 Hz, 1H), 8.03 (dd, J=8.5, 2.1 Hz, 1H), 7.72 (dq, J=8.5, 1.4 Hz, 1H).

### Step 2: Synthesis of N-(1-(3,4-dichlorophenyl)-2-(dimethylamino)ethyl)-3-nitro-4-(trifluoromethoxy)benzenesulfonamide (203 and 204

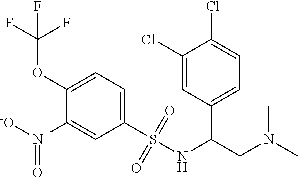

In a sealed tube, 3-nitro-4-(trifluoromethoxy)benzenesulfonyl chloride (97%, 811 mg, 2.57 mmol) was added to a stirred solution of 1-(3,4-dichlorophenyl)-N′,N′-dimethyl-ethane-1,2-diamine (100%, 600 mg, 2.57 mmol) and triethylamine (1076 μL, 7.72 mmol) in anhydrous DCM (26 mL). The solution was stirred at room temperature for 20 h. The reaction mixture was diluted with DCM. The organic layer was washed once with a saturated aqueous solution of NH₄Cl, once with a saturated solution of NaHCO₃, and once with a saturated aqueous solution of sodium chloride. The organic layer was dried trough a phase separator and concentrated under reduced pressure. The crude was purified by flash chromatography on silica gel using a gradient of methanol in dichloromethane from 0.2% to 5%. The residue was subjected to chiral separation (SFC). The resulting residues were dissolved in Et₂O and the resulting solution was added to pentane. The obtained suspensions were filtered, washed with pentane and dried under vacuum at 45° C. for 2 days to afford the racemic title compounds as a yellow powder.

974 mg of the racemic mixture was dissolved by sonication in MeOH (10 mL), ACN (4 mL), and i-PrOH (13 mL), to provide a solution having a concentration of 36.1 mg/mL. The solution was passed through a GHP filter (0.45 μm). 21 injections of the sample solution were performed on a Chiralpak IC column (250×20 mm, 5 μm) using 10% (i-PrOH+0.5% i-PrNH₂) in CO₂ as a mobile phase (Waters Prep SFC200, flow rate=50 mL/min, T=40° C., P=130 bar, UV detection at 254 nm) to afford the separated enantiomers. The separated enantiomers were dried under vacuum at 45° C. for 18 h to afford the title compounds each as a white powder.

Enantiomer I (203): (228.5 mg, 18% yield, 96.1% purity, tᵣ=1.51 min). LCMS (Method C): m/z found 502 [M+H]⁺; ¹H-NMR (500 MHz, DMSO-d₆) δ (ppm) 8.48-9.10 (m, 1H), 8.28 (d, J=2.2 Hz, 1H), 8.01 (dd, J=8.6, 2.2 Hz, 1H), 7.79 (br d, J=8.6 Hz, 1H), 7.41 (d, J=1.0 Hz, 1H), 7.39 (d, J=8.3 Hz, 1H), 7.18 (dd, J=8.3, 2.0 Hz, 1H), 4.29-4.67 (m, 1H), 2.42-2.57 (m, 1H), 1.75-2.41 (m, 7H).

Enantiomer II (204): (215.1 mg, 17% yield, 97% purity, tᵣ=1.5 min). LCMS (Method C): m/z found 502 [M+H]⁺; ¹H-NMR (500 MHz, DMSO-d₆) δ (ppm) 8.48-9.10 (m, 1H), 8.28 (d, J=2.2 Hz, 1H), 8.01 (dd, J=8.6, 2.2 Hz, 1H), 7.79 (br d, J=8.6 Hz, 1H), 7.41 (d, J=1.0 Hz, 1H), 7.39 (d, J=8.3 Hz, 1H), 7.18 (dd, J=8.3, 2.0 Hz, 1H), 4.29-4.67 (m, 1H), 2.42-2.57 (m, 1H), 1.75-2.41 (m, 7H).

### Example 82: N-(2-(dimethylamino)-1-(3-(trifluoromethyl)phenyl)ethyl)-3-nitro-4-(trifluoromethoxy)benzenesulfonamide (205)

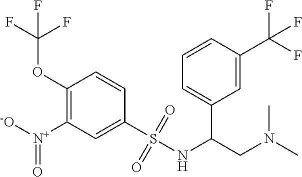

In a sealed tube, a solution of 3-nitro-4-(trifluoromethoxy)benzenesulfonyl chloride (97%, 102 mg, 0.323 mmol) in was added to a stirred solution of N′,N′-dimethyl-1-[3-(trifluoromethyl)phenyl]ethane-1,2-diamine (95%, 75 mg, 0.323 mmol) and triethylamine (135 μL, 0.969 mmol) in DCM (3.2 mL). The solution was stirred at rt for 5 h and was diluted with water. The aqueous layer was extracted twice with DCM, the combined organic layers were washed with a saturated aqueous solution of NH₄Cl, a saturated aqueous solution of NaHCO₃ and a saturated aqueous solution of NaCl, dried using a phase separator and concentrated. The crude was purified by flash chromatography on silica gel using a gradient of MeOH in DCM from 0.2% to 5%. The desired fraction were concentrated. The residue was dissolved in 2 mL of diethyl ether and 300 μL of a solution of 2 N HCl in Et₂O was added. The mixture was stirred for 1 h at rt and the resulting suspension was filtered, washed with diethyl ether and pentane and dried under vacuum at 40° C. for 18 h to afford the title compound as a beige powder (113.2 mg, 63% yield, 96.7% purity, tᵣ=1.49 min). LCMS (Method C): m/z found 502 [M+H]⁺; ¹H-NMR (DMSO-d₆, 500 MHz): δ (ppm) 9.82 (br s, 1H), 9.39 (br d, J=9.8 Hz, 1H), 8.17 (d, J=2.2 Hz, 1H), 7.95 (dd, J=8.7, 2.3 Hz, 1H), 7.62-7.65 (m, 2H), 7.60 (d, J=7.6 Hz, 1H), 7.44 (d, J=7.8 Hz, 1H), 7.31-7.37 (m, 1H), 5.06 (br t, J=11.5 Hz, 1H), 3.49 (br t, J=12.5 Hz, 1H), 3.23-3.29 (m, 1H), 2.93 (br s, 3H), 2.87 (br s, 3H).

### Example 83: N-(1-(4-chlorophenyl)-2-(dimethylamino)ethyl)-3-nitro-4-(trifluoromethoxy)benzenesulfonamide (206)

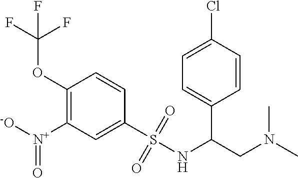

In a sealed tube, a solution of 3-nitro-4-(trifluoromethoxy)benzenesulfonyl chloride (115 mg, 0.377 mmol) in was added to a stirred solution of 1-(4-chlorophenyl)-N′,N′-dimethyl-ethane-1,2-diamine (75 mg, 0.377 mmol) and triethylamine (158 μL, 1.13 mmol) in DCM (3.5 mL). The solution was stirred at rt for 5 h and was diluted with water. The aqueous layer was extracted twice with DCM, the combined organic layers were washed with a saturated aqueous solution of NH₄Cl, a saturated aqueous solution of NaHCO₃ and a saturated aqueous solution of NaCl, dried using a phase separator and concentrated. The crude was purified by flash chromatography on silica gel using a gradient of MeOH in DCM from 0.2% to 5%. The desired fractions were concentrated. The residue was triturated in pentane, filtered, washed with pentane and dried under vacuum at 40° C. for 18 h to afford the title compound as a beige powder (113 mg, 63% yield, 98.2% purity, tᵣ=1.41 min). LCMS (Method C): m/z found 468 [M+H]⁺; ¹H-NMR (DMSO-d₆, 500 MHz): δ (ppm) 8.65 (br s, 1H), 8.26 (d, J=2.2 Hz, 1H), 8.00 (dd, J=8.7, 2.3 Hz, 1H), 7.77 (dd, J=8.7, 1.3 Hz, 1H), 7.17 (s, 4H), 4.40 (dd, J=9.2, 5.7 Hz, 1H), 2.48-2.55 (m, 1H), 2.23 (dd, J=12.7, 5.9 Hz, 1H), 2.03 (s, 6H).

### Example 84: 4-(cyclopropylmethoxy)-N-(1-(3,4-dichlorophenyl)-2-(dimethylamino)ethyl)benzenesulfonamide (207)

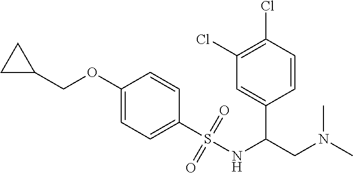 [SMILES: CN(C)CC(NS(=O)(=O)c1ccc(OCC2CC2)cc1)c1ccc(Cl)c(Cl)c1]

In a sealed vial under nitrogen, to a stirred solution of N-[1-(3,4-dichlorophenyl)-2-(dimethylamino)ethyl]-4-hydroxy-benzenesulfonamide (97%, 70 mg, 0.174 mmol) and (bromomethyl)cyclopropane (19 μL, 0.191 mmol) in anhydrous DMF (12 mL) was added dipotassium carbonate (144 mg, 1.04 mmol) and potassium iodide (14 mg, 0.0868 mmol), the mixture was stirred at 80° C. for 18 h. Water (9 mL) was added and the aqueous layer was extracted with ethyl acetate (2×10 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was triturated in 2 M hydrogen chloride in diethyl ether (0.87 mL, 1.74 mmol), filtered, washed with diethyl ether and dried under vacuum at 50° C. for 21 h to afford the title compound as a white powder (53.9 mg, 64% yield, 99.2% purity, tᵣ=1.46 min). LCMS (Method C): m/z found 443.2 [M+H]⁺; ¹H-NMR (500 MHz, DMSO-d₆) δ (ppm) 9.26-9.91 (m, 1H), 8.52 (br d, J=9.5 Hz, 1H), 7.32-7.43 (m, 4H), 7.16-7.26 (m, 1H), 6.78 (d, J=8.8 Hz, 2H), 4.45-5.21 (m, 1H), 3.67-3.86 (m, 2H), 3.33-3.38 (m, 1H), 3.13-3.26 (m, 1H), 2.67-2.99 (m, 6H), 1.11-1.28 (m, 1H), 0.24-0.68 (m, 4H).

### Example 85: N-(1-(3,4-dichlorophenyl)-2-(dimethylamino)ethyl)-4-isopropoxybenzenesulfonamide (208)

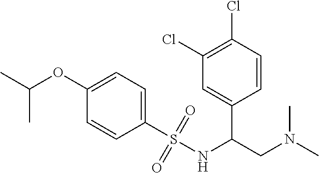 [SMILES: CC(C)Oc1ccc(S(=O)(=O)NC(CN(C)C)c2ccc(Cl)c(Cl)c2)cc1]

In a sealed tube, 4-(propan-2-yloxy)benzenesulfonyl chloride (98%, 54 mg, 0.224 mmol) was added to a stirred solution of 1-(3,4-dichlorophenyl)-N²,N²-dimethylethane-1,2-diamine (95%, 50 mg, 0.204 mmol) and triethylamine (85 μL, 0.611 mmol) in DCM (2 mL). The solution was stirred at room temperature for 18 h. Water (5 mL) was added and the aqueous layer was extracted with dichloromethane (1×10 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was triturated in 2 M hydrogen chloride in diethyl ether (1.0 mL, 2.04 mmol), filtered, washed with diethyl ether and dried under vacuum at 50° C. for 3 days to afford the title compound as a white powder (30.6 mg, 32% yield, 100% purity, tᵣ=1.46 min). LCMS (Method C): m/z found 431 [M+H]⁺; ¹H-NMR (DMSO-d₆, 500 MHz): δ (ppm) 9.52 (br d, J=4.4 Hz, 1H), 8.49 (br d, J=10.3 Hz, 1H), 7.32-7.46 (m, 4H), 7.21 (dd, J=8.3, 2.0 Hz, 1H), 6.72-6.84 (m, 2H), 4.70-4.86 (m, 1H), 4.58 (dt, J=12.0, 6.1 Hz, 1H), 3.31 (s, 2H), 2.68-3.04 (m, 6H), 1.24 (dd, J=6.1, 2.4 Hz, 6H).

### Example 86: N-(1-(3,4-dichlorophenyl)-2-(dimethylamino)ethyl)-4-isopropoxybenzenesulfonamide (209)

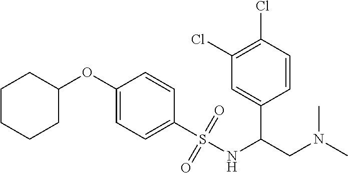 [SMILES: CN(C)CC(NS(=O)(=O)c1ccc(OC2CCCCC2)cc1)c1ccc(Cl)c(Cl)c1]

In a sealed vial under nitrogen, to a stirred solution of N-[1-(3,4-dichlorophenyl)-2-(dimethylamino)ethyl]-4-hydroxy-benzenesulfonamide (95%, 200 mg, 0.488 mmol) and bromocyclohexane (120 μL, 0.976 mmol) in anhydrous DMF (4.0671 mL) was added K₂CO₃ (270 mg, 1.95 mmol) and potassium iodide (16 mg, 0.0976 mmol), the mixture was stirred at rt overnight, then at 110° C. for 24 h, and overnight at 130° C. Additional bromocyclohexane (0.12 mL, 0.976 mmol), K₂CO₃ (270 mg, 1.95 mmol), and KI (16 mg, 0.0976 mmol) were added and the mixture was stirred for two days at 90° C. Additional bromocyclohexane (0.060 mL, 0.488 mmol) and K₂CO₃ (67 mg, 0.488 mmol) were added at rt and the reaction mixture was stirred at 140° C. for 1 h under microwave irradiation, and overnight once more at 90° C. Water was added and the aqueous layer was extracted twice with ethyl acetate. The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%, once more using a gradient of methanol (0.7 N NH₃) in DCM from 0.4% to 2%, and a final time by reverse-phase chromatography using a gradient of acetonitrile in water from 0% to 100% (0.1% TFA). The desired fractions were gathered, basified, extracted using DCM, filtered through phase separator, concentrated in vacuo, then dissolved in Et₂O, and 2 M HCl in Et₂O (0.98 mL, 1.95 mmol) was added. The mixture was stirred 3 h at rt and concentrated in vacuo to afford the title compound as a white powder (20 mg, 8% yield, 96.3% purity, tᵣ=1.75 min). LCMS (Method C): m/z found 471 [M+H]⁺; ¹H-NMR (500 MHz, DMSO-d₆) δ (ppm) 9.72-9.34 (m, 1H), 8.48 (br d, J=10.5 Hz, 1H), 7.39 (dd, J=12.5, 8.6 Hz, 4H), 7.20 (dd, J=8.3, 2.0 Hz, 1H), 6.78 (d, J=9.0 Hz, 2H), 4.86-4.67 (m, 1H), 4.40-4.22 (m, 1H), 3.31 (s, 2H), 3.00-2.72 (m, 6H), 1.97-1.17 (m, 10H).

### Example 87: 4-(cyclopentyloxy)-N-(1-(3,4-dichlorophenyl)-2-(dimethylamino)ethyl)benzenesulfonamide (210)

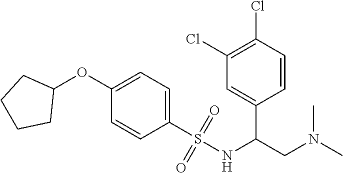 [SMILES: CN(C)CC(NS(=O)(=O)c1ccc(OC2CCCC2)cc1)c1ccc(Cl)c(Cl)c1]

In a sealed vial under nitrogen, to a stirred solution of N-[1-(3,4-dichlorophenyl)-2-(dimethylamino)ethyl]-4-hydroxy-benzenesulfonamide (95%, 70 mg, 0.171 mmol) and bromocyclopentane (95%, 121 μL, 1.02 mmol) in anhydrous DMF (1.75 mL) was added K₂CO₃ (142 mg, 1.02 mmol) and KI (14 mg, 0.0854 mmol), the mixture was stirred at 80° C. for 18 h, then additional water (9 mL) was added, and the aqueous layer was extracted with ethyl acetate (2×10 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was triturated in pentane, filtered, washed with pentane and dried under vacuum at 70° C. for 3 days to afford the title compound as a white powder (58.1 mg, 74% yield, 99.3% purity, tᵣ=1.67 min). LCMS (Method C): m/z found 457.2 [M+H]⁺; ¹H-NMR (500 MHz, DMSO-d₆) δ (ppm) 7.91 (br d, J=7.8 Hz, 1H), 7.46 (d, J=8.8 Hz, 2H), 7.37 (d, J=8.3 Hz, 1H), 7.31 (d, J=2.0 Hz, 1H), 7.13 (dd, J=8.3, 2.0 Hz, 1H), 6.83 (d, J=8.8 Hz, 2H), 4.87-4.74 (m, 1H), 4.28 (br d, J=7.3 Hz, 1H), 2.44-2.23 (m, 2H), 2.05 (s, 6H), 1.96-1.82 (m, 2H), 1.79-1.46 (m, 6H).

### Example 88: N-(1-(3,4-dichlorophenyl)-2-(dimethylamino)ethyl)-4-(pyridin-2-yloxy)benzenesulfonamide (211)

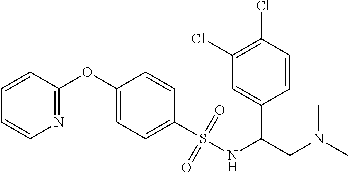 [SMILES: CN(C)CC(NS(=O)(=O)c1ccc(Oc2ccccn2)cc1)c1ccc(Cl)c(Cl)c1]

In a sealed vial under nitrogen, to a stirred solution of N-[1-(3,4-dichlorophenyl)-2-(dimethylamino)ethyl]-4-hydroxy-benzenesulfonamide (95%, 50 mg, 0.122 mmol) and 2-bromopyridine (17 μL, 0.183 mmol) in anhydrous DMSO (1000 μL) were added pyridine-2-carboxylic acid (97%, 6.2 mg, 0.0488 mmol), K₃PO₄ (52 mg, 0.244 mmol) and CuI (4.6 mg, 0.0244 mmol). The vial was evacuated and backfilled to N₂ (3 cycles). The mixture was stirred at 90° C. for 46 h. Water and ethyl acetate were added and the aqueous layer was extracted twice with ethyl acetate. The combined organic layers were washed with a saturated aqueous solution of sodium chloride, dried over sodium sulfate, filtered through a pad of dicalite and concentrated under reduced pressure. The crude was purified by flash chromatography on silica gel using a gradient of methanol in dichloromethane from 0% to 5%. The resulting oil was purified by reverse-phase preparative chromatography, using a gradient of acetonitrile in water from 0% to 100% (0.10% TFA in water). The desired fractions were combined and concentrated. The resulting oil was solubilized in Et₂O and the homogenous solution was added to 2 M hydrogen chloride (0.61 mL, 1.22 mmol) in Et₂O. The resulting suspension was stirred at rt for 4 h. The precipitate formed was filtered, washed with Et₂O and dried under vacuum at 45° C. overnight to afford the title compound as a white powder (25.5 mg, 41.6% yield, 99.8% purity, tᵣ=1.81 min). LCMS (Method C): m/z found 466.2 [M+H]⁺; ¹H-NMR (600 MHz, DMSO-d₆, 300K) δ (ppm) 9.61 (br s, 1H), 8.73 (br d, J=10.0 Hz, 1H), 8.23 (ddd, J=5.0, 2.0, 0.7 Hz, 1H), 7.92 (ddd, J=8.3, 7.3, 2.1 Hz, 1H), 7.54-7.61 (m, 2H), 7.51 (d, J=2.1 Hz, 1H), 7.43 (d, J=8.2 Hz, 1H), 7.20-7.24 (m, 2H), 7.03-7.07 (m, 2H), 7.01 (d, J=8.2 Hz, 1H), 4.85 (br d, J=3.2 Hz, 1H), 3.34-3.43 (m, 1H), 3.18-3.28 (m, 1H), 2.78-2.95 (m, 6H).

### Example 89: N-(1-(3,4-dichlorophenyl)-2-(dimethylamino)ethyl)-4-ethylbenzenesulfonamide (212)

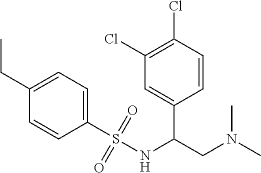 [SMILES: CCc1ccc(S(=O)(=O)NC(CN(C)C)c2ccc(Cl)c(Cl)c2)cc1]

In a sealed tube, a solution of 4-ethylbenzenesulfonyl chloride (60 mg, 0.293 mmol) in DCM (500 μL) was added to a stirred solution of 1-(3,4-dichlorophenyl)-N²,N²-dimethylethane-1,2-diamine (75 mg, 0.322 mmol) and triethylamine (163 μL, 1.17 mmol) in DCM (1 mL). The solution was stirred at rt for 4 h and was diluted with half saturated aqueous NaHCO₃. The aqueous layer was extracted with DCM, the combined organic layers were washed with brine, dried over sodium sulfate, filtered and concentrated. The crude was purified by flash chromatography on silica gel using a gradient of (MeOH+2% NH₄OH) in DCM from 0.2% to 5%. The desired fractions were concentrated and triturated in pentane. The obtained suspension was filtered, the residue was washed with pentane and dried under reduced pressure at 45° C. for 16 h to afford the title compound as a white powder. (37 mg, 31% yield, 99% purity, tᵣ=1.64 min). LCMS (Method C): m/z found 401 [M+H]⁺; ¹H-NMR (500 MHz, DMSO-d₆) δ (ppm) 8.01 (br s, 1H), 7.47 (d, J=8.31 Hz, 2H), 7.35 (d, J=8.31 Hz, 1H), 7.29 (d, J=1.96 Hz, 1H), 7.19 (d, J=8.07 Hz, 2H), 7.12 (dd, J=2.08, 8.19 Hz, 1H), 4.29 (br s, 1H), 2.59 (q, J=7.58 Hz, 2H), 2.37 (dd, J=7.83, 12.47 Hz, 1H), 2.29 (dd, J=7.21, 12.59 Hz, 1H), 2.04 (s, 6H), 1.15 (t, J=7.58 Hz, 3H).

### Example 90: N-(1-(3,4-dichlorophenyl)-2-(dimethylamino)ethyl)-4-isopropylbenzenesulfonamide (213)

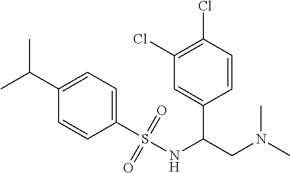 [SMILES: CC(C)c1ccc(S(=O)(=O)NC(CN(C)C)c2ccc(Cl)c(Cl)c2)cc1]

In a sealed tube, 4-(propan-2-yl)benzenesulfonyl chloride (96%, 40 μL, 0.214 mmol) was added to a stirred solution of 1-(3,4-dichlorophenyl)-N′,N′-dimethyl-ethane-1,2-diamine (90%, 56 mg, 0.214 mmol) and triethylamine (90 μL, 0.643 mmol) in anhydrous DCM (2 mL). The solution was stirred at room temperature for 3 h. The reaction mixture was diluted with DCM. The organic layer was washed once with a saturated aqueous solution of NH₄Cl, once with a saturated solution of NaHCO₃ and once with a saturated aqueous solution of sodium chloride. The organic layer was dried trough a phase separator and concentrated under reduced pressure. The crude was purified by flash chromatography on silica gel using a gradient of methanol in dichloromethane from 0.2% to 5%. The residue was dissolved in 2 mL of Et₂O and 300 μL of a solution of HCl at 2 N in Et₂O was added. The mixture was stirred for 1 h and the resulting suspension was filtered, washed with Et₂O and pentane. The resulting powder dried under vacuum at 40° C. for 18 h to afford the title compound as a white powder (57.2 mg, 57% yield, 96.1% purity, tᵣ=1.57 min). LCMS (Method C): m/z found 415 [M+H]⁺; ¹H-NMR (600 MHz, DMSO-d₆) δ (ppm) 9.66 (br s, 1H), 8.66 (br d, J=9.2 Hz, 1H), 7.42 (d, J=8.5 Hz, 2H), 7.40 (d, J=2.1 Hz, 1H), 7.32 (d, J=8.2 Hz, 1H), 7.18 (dd, J=8.4, 2.1 Hz, 1H), 7.14 (d, J=8.4 Hz, 2H), 4.80 (br t, J=11.0 Hz, 1H), 3.39-3.32 (m, 1H), 3.25-3.13 (m, 1H), 2.96-2.76 (m, 7H), 1.12 (d, J=6.9 Hz, 6H).

### Example 91: 4-butyl-N-(1-(3,4-dichlorophenyl)-2-(dimethylamino)ethyl)benzenesulfonamide (214)

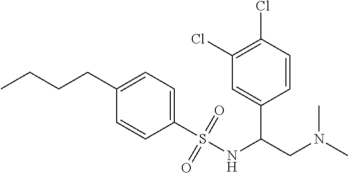 [SMILES: CCCCc1ccc(S(=O)(=O)NC(CN(C)C)c2ccc(Cl)c(Cl)c2)cc1]

In a sealed tube, 4-butylbenzenesulfonyl chloride (95%, 47 μL, 0.234 mmol) was added to a stirred solution of 1-(3,4-dichlorophenyl)-N′,N′-dimethyl-ethane-1,2-diamine (99%, 50 mg, 0.212 mmol) and triethylamine (89 μL, 0.637 mmol) in anhydrous DCM (2.2 mL). The solution was stirred at room temperature for 2 days. Water (5 mL) was added and the aqueous layer was extracted with ethyl acetate (2×7 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was triturated in 2 M hydrogen chloride in diethyl ether (1.1 mL, 2.12 mmol), filtered, washed with diethyl ether and dried under vacuum at 70° C. for 18 h to afford the title compound as a white powder (82.9 mg, 83% yield, 99% purity, tᵣ=1.74 min). LCMS (Method C): m/z found 429.3 [M+H]⁺; ¹H-NMR (500 MHz, DMSO-d₆) δ (ppm) 9.70 (br s, 1H), 8.68 (br d, J=9.5 Hz, 1H), 7.47-7.39 (m, 3H), 7.34 (d, J=8.3 Hz, 1H), 7.20 (dd, J=8.3, 2.0 Hz, 1H), 7.10 (d, J=8.3 Hz, 2H), 4.80 (br s, 1H), 3.48-3.09 (m, 2H), 3.01-2.68 (m, 6H), 2.58-2.42 (m, 2H), 1.56-1.38 (m, 2H), 1.36-1.10 (m, 2H), 0.90 (t, J=7.3 Hz, 3H).

### Example 92: N-(1-(3,4-dichlorophenyl)-2-(dimethylamino)ethyl)-4-pentylbenzenesulfonamide (215)

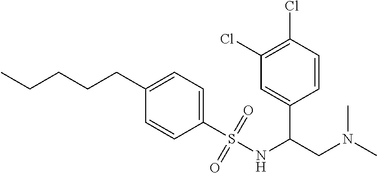 [SMILES: CCCCCc1ccc(S(=O)(=O)NC(CN(C)C)c2ccc(Cl)c(Cl)c2)cc1]

In a sealed tube, 4-pentylbenzenesulfonyl chloride (71 mg, 0.290 mmol) was added to a stirred solution of triethylamine (0.12 mL, 0.869 mmol) and 1-(3,4-dichlorophenyl)-N²,N²-dimethylethane-1,2-diamine (90%, 75 mg, 0.290 mmol) in anhydrous DCM (2.5376 mL). The solution was stirred at room temperature overnight. Water (6 mL) was added and the aqueous layer was extracted with dichloromethane (2×6 mL). The combined organic layers were washed with a saturated aqueous solution of sodium chloride, dried over sodium sulfate, filtered and concentrated under reduced pressure. The crude was purified by flash chromatography on silica gel (column 12 g, 15 μm) using a gradient of 0.7 N ammonia in methanol in dichloromethane from 0% to 5%. The residue was dissolved in Et₂O and a solution of HCl at 2 N in Et₂O was added. The mixture was stirred for 15 min and the resulting suspension was filtered, washed with Et₂O and pentane. The resulting solid was dried under vacuum at 45° C. for 18 h to afford the title compound as a white solid (45 mg, 32.349% yield, 99.9% purity, tᵣ=2.04 min). LCMS (Method C): m/z found 443 [M+H]⁺; ¹H-NMR (500 MHz, DMSO-d₆) δ (ppm) 9.67 (br s, 1H), 8.66 (br d, J=10.0 Hz, 1H), 7.49-7.39 (m, 3H), 7.34 (d, J=8.3 Hz, 1H), 7.20 (dd, J=8.3, 2.0 Hz, 1H), 7.10 (d, J=8.1 Hz, 2H), 4.95-4.64 (m, 1H), 3.47-3.09 (m, 2H), 3.02-2.73 (m, 6H), 2.54-2.46 (m, 2H), 1.47 (quin, J=7.6 Hz, 2H), 1.38-1.14 (m, 4H), 0.88 (t, J=7.2 Hz, 3H).

### Example 93: N-(1-(3,4-dichlorophenyl)-2-(dimethylamino)ethyl)-4-(3,6-dihydro-2H-pyran-4-yl)benzenesulfonamide (216)

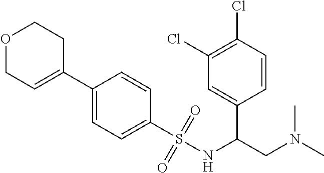 [SMILES: CN(C)CC(NS(=O)(=O)c1ccc(C2=CCOCC2)cc1)c1ccc(Cl)c(Cl)c1]

A tube sealed was charged with 4-bromo-N-[1-(3,4-dichlorophenyl)-2-(dimethylamino)ethyl]benzenesulfonamide (100%, 150 mg, 0.332 mmol), K₂CO₃ (138 mg, 0.996 mmol) and XPhos Pd G2 (42 mg, 0.0578 mmol) in water (0.7632 mL) and 1,4-dioxane (3.2802 mL). The reaction mixture was degassed with argon for 15 min and 4-(4,4,5,5-tetramethyl-1,3,2-dioxaborolan-2-yl)-3,6-dihydro-2H-pyran (97%, 108 mg, 0.498 mmol) was added. The reaction mixture was stirred at 100° C. overnight. The suspension was filtered through dicalite and washed with DCM. The filtrate was washed with water two times, and once with a saturated aqueous solution of sodium chloride, dried over sodium sulfate, filtered and concentrated under reduced pressure. The crude was purified by flash chromatography on silica gel (column gold 12 g, 15 μm) using a gradient of 0.7 N ammonia in methanol in dichloromethane from 0.5% to 5%. The desired fractions were combined and concentrated to afford the title compound as an off-white solid (53.2 mg, 31% yield, 96.1% purity, tᵣ=1.23 min). LCMS (Method C): m/z found 455.2 [M+H]⁺; ¹H-NMR (500 MHz, DMSO-d₆, 300K) δ (ppm) 8.08 (br s, 1H), 7.49-7.56 (m, 2H), 7.42 (d, J=8.6 Hz, 2H), 7.36 (d, J=8.3 Hz, 1H), 7.29 (d, J=2.0 Hz, 1H), 7.13 (dd, J=8.3, 2.0 Hz, 1H), 6.33 (br s, 1H), 4.32 (br d, J=4.9 Hz, 1H), 4.24 (q, J=2.9 Hz, 2H), 3.82 (t, J=5.5 Hz, 2H), 1.74-2.47 (m, 10H).

### Example 94: 4-chloro-N-(1-(3,4-dichlorophenyl)-2-(dimethylamino)ethyl)-3-fluorobenzenesulfonamide (217)

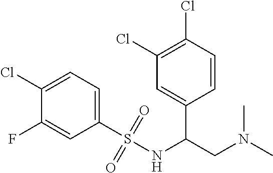 [SMILES: CN(C)CC(NS(=O)(=O)c1ccc(Cl)c(F)c1)c1ccc(Cl)c(Cl)c1]

In a sealed tube, 4-chloro-3-fluorobenzenesulfonyl chloride (95%, 70 mg, 0.291 mmol) was added to a stirred solution of 1-(3,4-dichlorophenyl)-N′,N′-dimethyl-ethane-1,2-diamine (95%, 65 mg, 0.265 mmol) and triethylamine (111 μL, 0.795 mmol) in DCM (2.6 mL). The solution was stirred at room temperature for 18 h. Water (5 mL) was added and the aqueous layer was extracted with dichloromethane (1×10 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was triturated in pentane, filtered, washed with pentane and dried under vacuum at 50° C. for 18 h to afford the title compound as an off-white powder (94.9 mg, 84% yield, 100% purity, tᵣ=1.57 min). LCMS (Method C): m/z found 424.9 [M+H]⁺; ¹H-NMR (500 MHz, DMSO-d₆) δ (ppm) 8.40 (br s, 1H), 7.64 (dd, J=7.46, 8.19 Hz, 1H), 7.48 (dd, J=1.96, 9.05 Hz, 1H), 7.41 (t, J=8.48 Hz, 2H), 7.31 (d, J=1.96 Hz, 1H), 7.17 (dd, J=1.96, 8.31 Hz, 1H), 4.39 (t, J=7.58 Hz, 1H), 2.46 (dd, J=8.44, 12.59 Hz, 1H), 2.31 (dd, J=6.72, 12.59 Hz, 1H), 2.08 (s, 6H).

### Example 95: N-(1-(3,4-dichlorophenyl)-2-(dimethylamino)ethyl)-4-(pyridin-2-yl)benzenesulfonamide (218)

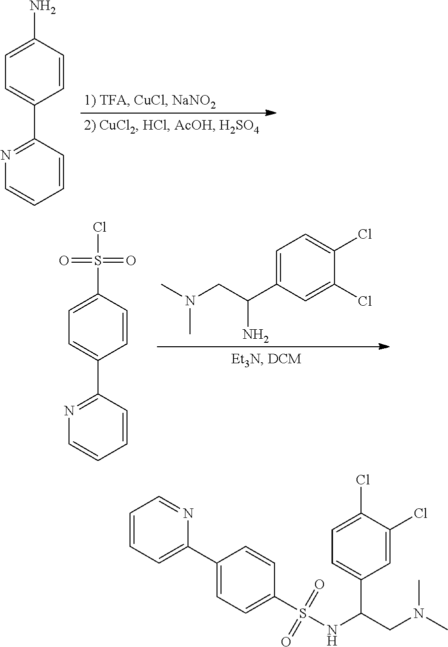

### Step 1: Synthesis of 4-(2-pyridyl)benzenesulfonyl chloride

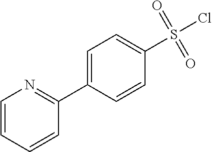 [SMILES: O=S(=O)(Cl)c1ccc(-c2ccccn2)cc1]

In a three necked round bottomed flask equipped with a thermometer, a solution of 4-(2-pyridyl)aniline (98%, 300 mg, 1.76 mmol) in TFA (5.9922 mL) was stirred at 0° C. Copper(I) chloride (5.3 mg, 0.0529 mmol) was added, the mixture was stirred at 0° C. for 5 min, then a solution of sodium nitrite (154 mg, 2.20 mmol) in water (0.6392 mL) was added dropwise to keep the temperature below 0° C. The mixture was stirred at −5° C. for 30 min to afford Solution A. In a three necked round bottomed flask equipped with a thermometer, a solution of copper(II) dichloride (121 mg, 0.899 mmol) and concentrated hydrogen chloride (0.59 mL, 19.4 mmol) in acetic acid (5.9922 mL) and sulfuric acid (6.0 mL, 77.6 mmol) was stirred at −5° C. Solution A was added dropwise to keep the temperature below 5° C. The mixture was stirred at 0° C. for 5 min then a rt for 1 h and was extracted twice with DCM. The combined organic layers were dried over sodium sulfate, filtered and concentrated. The residue is washed again once with water and twice with a saturated solution of sodium bicarbonate to remove the acetic acid still present. The organic layers was washed with brine, dried over sodium sulfate, filtered and concentrated under reduced pressure to afford the title compound as a brown solid (214 mg, 47.858% yield, tᵣ=0.59 min). LCMS (Method F); ¹H-NMR (DMSO-d₆, 400 MHz) δ (ppm) 8.85-8.77 (m, 1H), 8.34 (t, J=7.9 Hz, 1H), 8.24 (d, J=8.1 Hz, 1H), 8.05-7.97 (m, 2H), 7.83-7.70 (m, 3H).

### Step 2: Synthesis of N-(1-(3,4-dichlorophenyl)-2-(dimethylamino)ethyl)-4-(pyridin-2-yl)benzenesulfonamide (218)

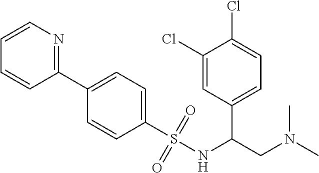 [SMILES: CN(C)CC(NS(=O)(=O)c1ccc(-c2ccccn2)cc1)c1ccc(Cl)c(Cl)c1]

In a sealed tube, triethylamine (0.13 mL, 0.965 mmol) was added to a stirred solution of 1-(3,4-dichlorophenyl)-N′,N′-dimethyl-ethane-1,2-diamine (75 mg, 0.322 mmol) and 4-(2-pyridyl)benzenesulfonyl chloride (82 mg, 0.324 mmol) in anhydrous DCM (2.8195 mL). The solution was stirred at room temperature overnight. Water (6 mL) was added and the aqueous layer was extracted with dichloromethane (2×6 mL). The combined organic layers were washed with a saturated aqueous solution of sodium chloride, dried over sodium sulfate, filtered and concentrated under reduced pressure. The crude was purified by flash chromatography on silica gel (column 25 g, 15 μm) using a gradient of methanol in dichloromethane from 0% to 5%. The desired fractions were combined and concentrated. The resulting oil was solubilized in DCM and the homogenous solution was added to pentane. The precipitate formed was filtered, washed with pentane and dried under vacuum at 40° C. for 18 h to afford the title compound as a white solid (76.6 mg, 52.753% yield, 99.8% purity, tᵣ=1.2 min). LCMS (Method C): m/z found 450.2 [M+H]⁺; ¹H-NMR (600 MHz, DMSO-d₆) δ (ppm) 8.70 (d, J=4.69 Hz, 1H), 8.18 (br s, 1H), 8.09-8.12 (m, 2H), 7.97-8.00 (m, 1H), 7.93 (dt, J=1.76, 7.70 Hz, 1H), 7.69-7.72 (m, 2H), 7.36-7.43 (m, 3H), 7.18 (dd, J=2.05, 8.36 Hz, 1H), 4.36 (br t, J=7.19 Hz, 1H), 2.40 (dd, J=8.00, 12.54 Hz, 1H), 2.32 (dd, J=7.12, 12.54 Hz, 1H), 2.04 (s, 6H).

### Example 96: N-(2-(dimethylamino)-1-(4-(trifluoromethyl)phenyl)ethyl)-4-(trifluoromethoxy)benzenesulfonamide (219)

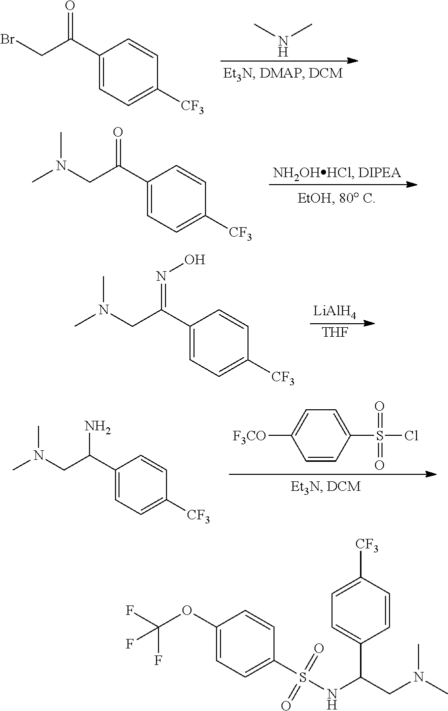

### Step 1: Synthesis of 2-(dimethylamino)-1-[4-(trifluoromethyl)phenyl]ethanone

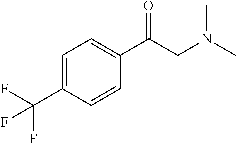 [SMILES: CN(C)CC(=O)c1ccc(C(F)(F)F)cc1]

In a round-bottomed flask, to a stirred solution of triethylamine (563 μL, 4.04 mmol) in anhydrous DCM (8.5 mL) at room temperature under nitrogen were added successively 2 M dimethylamine in THF (3.3 mL, 6.61 mmol), DMAP (22 mg, 0.183 mmol), and 2-bromo-1-[4-(trifluoromethyl)phenyl]ethanone (98%, 1.00 g, 3.67 mmol). The reaction mixture was stirred at room temperature for 18 h. The reaction mixture was quenched with water (20 mL) and ethyl acetate (20 mL) was added. The aqueous layer was extracted with ethyl acetate (1×15 mL). The combined organic layers were washed with a saturated aqueous solution of sodium chloride, dried over sodium sulfate, filtered, and concentrated under reduced pressure. The crude was purified by flash chromatography on silica gel using a gradient of methanol in dichloromethane from 2% to 5% to afford the expected compound as a brown oil (695 mg, 78% yield, tᵣ=0.72 min). LCMS (Method F): m/z found 232.2 [M+H]⁺; ¹H-NMR (DMSO-d₆, 400 MHz) δ (ppm) 8.24-8.09 (m, 2H), 7.89 (d, J=8.1 Hz, 2H), 3.80 (s, 2H), 2.25 (s, 6H).

### Step 2: Synthesis of 2-(dimethylamino)-1-[4-(trifluoromethyl)phenyl]ethanone oxime

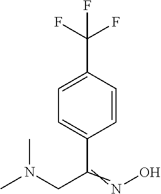 [SMILES: CN(C)CC(=NO)c1ccc(C(F)(F)F)cc1]

A sealed vial was charged with 2-(dimethylamino)-1-[4-(trifluoromethyl)phenyl]ethanone (695 mg, 3.01 mmol), hydroxylamine hydrochloride (418 mg, 6.01 mmol) and DIPEA (1.1 mL, 6.31 mmol) in anhydrous ethanol (14 mL). The reaction mixture was stirred at 80° C. for 18 h. The reaction mixture was concentrated under reduced pressure and dried under vacuum to afford the expected compound as a brown oil (1.655 g, quantitative yield, tᵣ=0.76 min). LCMS (Method F): m/z found 247.2 [M+H]⁺; 1H-NMR (DMSO-d₆, 400 MHz) δ (ppm) 9.28 (s, 1H), 8.18-7.70 (m, 4H), 3.45 (q, J=7.0 Hz, 2H), 2.15 (d, J=8.0 Hz, 6H).

### Step 3: Synthesis of N′,N′-dimethyl-1-[4-(trifluoromethyl)phenyl]ethane-1,2-diamine

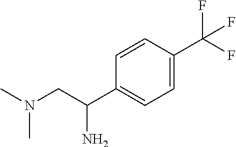 [SMILES: CN(C)CC(N)c1ccc(C(F)(F)F)cc1]

In a vial under atmospheric pressure, to a stirred solution of 2-(dimethylamino)-1-[4-(trifluoromethyl)phenyl]ethanone oxime (740 mg, 3.01 mmol) in anhydrous THF (13 mL) at 0° C. under nitrogen was added 2.4 M LAH in THF (3.1 mL, 7.51 mmol). The reaction mixture was stirred at 70° C. for 3 h. The reaction mixture was quenched at 0° C. with water (313 μL, same weight as LAH used), 1 M sodium hydroxide (302 μL, 0.302 mmol) and water (three times the weight of LAH used) were added. The resulting heterogenous mixture was stirred at room temperature for 15 min, dried over sodium sulfate, filtered, rinsed with THF, and concentrated under reduced pressure. The crude was purified by flash chromatography on silica gel using a gradient of ammoniacal methanol in dichloromethane from 3% to 10% to afford the title compound as a brown oil (154 mg, 21% yield, tᵣ=0.42 min). LCMS (Method F): m/z found 233.2 [M+H]⁺; ¹H-NMR (DMSO-d₆, 400 MHz) δ (ppm) 7.71-7.59 (m, 4H), 4.07 (dd, J=9.1, 5.2 Hz, 1H), 2.38-2.19 (m, 2H), 2.19 (s, 6H), 2.13 (d, J=6.4 Hz, 2H).

### Step 4: Synthesis of N-(2-(dimethylamino)-1-(4-(trifluoromethyl)phenyl)ethyl)-4-(trifluoromethoxy)benzenesulfonamide (219)

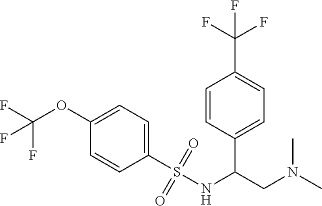 [SMILES: CN(C)CC(NS(=O)(=O)c1ccc(OC(F)(F)F)cc1)c1ccc(C(F)(F)F)cc1]

In a sealed tube, 4-(trifluoromethoxy)benzenesulfonyl chloride (48 μL, 0.284 mmol) was added to a stirred solution of N′,N′-dimethyl-1-[4-(trifluoromethyl)phenyl]ethane-1,2-diamine (60 mg, 0.258 mmol) and triethylamine (108 μL, 0.775 mmol) in DCM (2.5 mL). The solution was stirred at room temperature for 18 h. Water (5 mL) was added and the aqueous layer was extracted with dichloromethane (1×10 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was triturated in pentane, filtered, washed with pentane and dried under vacuum at 70° C. for 18 h to afford the title compound as a white powder (88.5 mg, 70% yield, 99.9% purity, tᵣ=1.47 min). LCMS (Method C): m/z found 457 [M+H]⁺; ¹H-NMR (500 MHz, DMSO-d₆) δ (ppm) 8.41 (br d, J=1.5 Hz, 1H), 7.77-7.61 (m, 2H), 7.45 (d, J=8.1 Hz, 2H), 7.37-7.28 (m, 4H), 4.42 (br s, 1H), 2.46 (dd, J=12.5, 8.3 Hz, 1H), 2.31 (dd, J=12.6, 6.7 Hz, 1H), 2.05 (s, 6H).

### Example 97: N-(1-(3,4-dichlorophenyl)-2-(dimethylamino)ethyl)-4-(3-fluorophenoxy)benzenesulfonamide (220)

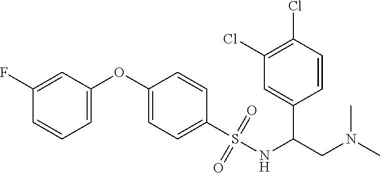 [SMILES: CN(C)CC(NS(=O)(=O)c1ccc(Oc2cccc(F)c2)cc1)c1ccc(Cl)c(Cl)c1]

In a sealed tube, 4-(3-fluorophenoxy)benzenesulfonyl chloride (95%, 46 μL, 0.236 mmol) was added to a stirred solution of 1-(3,4-dichlorophenyl)-N′,N′-dimethyl-ethane-1,2-diamine (50 mg, 0.214 mmol) and triethylamine (90 μL, 0.643 mmol) in DCM (2 mL). The solution was stirred at room temperature for 18 h. Water (5 mL) was added and the aqueous layer was extracted with dichloromethane (1×10 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 4%. The residue was triturated in pentane, filtered, washed with pentane and dried under vacuum at 50° C. for 3 days to afford the title compound as a white powder (81.3 mg, 78% yield, 99.7% purity, tᵣ=1.65 min). LCMS (Method C): m/z found 483 [M+H]⁺; ¹H-NMR (500 MHz, DMSO-d₆) δ (ppm) 8.13 (br d, J=7.6 Hz, 1H), 7.59 (d, J=8.8 Hz, 2H), 7.54-7.46 (m, 1H), 7.43 (d, J=8.1 Hz, 1H), 7.36 (d, J=2.0 Hz, 1H), 7.17 (dd, J=8.3, 2.0 Hz, 1H), 7.09 (td, J=8.6, 2.0 Hz, 1H), 6.98 (d, J=9.0 Hz, 2H), 6.94 (t, J=2.3 Hz, 1H), 6.85 (dd, J=8.3, 2.0 Hz, 1H), 4.34 (br d, J=7.1 Hz, 1H), 2.45-2.26 (m, 2H), 2.07 (s, 6H).

### Example 98: N-(1-(3,4-dichlorophenyl)-2-(dimethylamino)ethyl)-4-(4-fluorophenoxy)benzenesulfonamide (221)

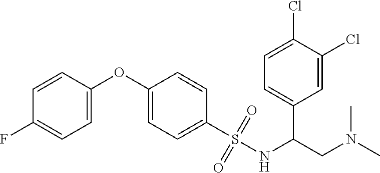 [SMILES: CN(C)CC(NS(=O)(=O)c1ccc(Oc2ccc(F)cc2)cc1)c1ccc(Cl)c(Cl)c1]

In a round bottomed flask under nitrogen, 4-(4-fluorophenoxy)benzenesulfonyl chloride (95%, 65 mg, 0.214 mmol) was added to a stirred solution of 1-(3,4-dichlorophenyl)-N²,N²-dimethylethane-1,2-diamine (50 mg, 0.214 mmol) and triethylamine (0.12 mL, 0.858 mmol) in anhydrous DCM (1.4298 mL). The solution was stirred at rt overnight then DCM and an half saturated solution of NaHCO₃ were added and the aqueous layer was extracted twice with DCM. The combined organic layers were filtered through phase separator, then concentrated in vacuo and purified by flash chromatography on silica gel using a gradient of MeOH in DCM from 0.4% to 4% to afford the title compound as a white powder (55 mg, 53% yield, 100% purity, tᵣ=1.63 min). LCMS (Method C): m/z found 483 [M+H]⁺; ¹H-NMR (500 MHz, DMSO-d₆) δ (ppm) 8.31-7.91 (m, 1H), 7.54 (d, J=8.8 Hz, 2H), 7.42 (d, J=8.3 Hz, 1H), 7.36-7.27 (m, 3H), 7.15 (dd, J=8.3, 1.7 Hz, 1H), 7.12-7.02 (m, 2H), 6.87 (d, J=8.8 Hz, 2H), 4.33 (br d, J=1.5 Hz, 1H), 2.47-2.24 (m, 2H), 2.07 (br s, 6H).

### Example 99: N-(1-(3,4-dichlorophenyl)-2-(dimethylamino)ethyl)-4-nitro-3-(trifluoromethoxy)benzenesulfonamide (222)

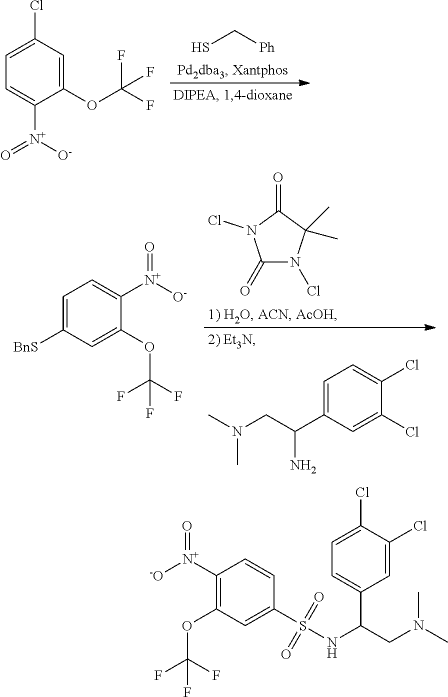

### Step 1: Synthesis of 4-benzylsulfanyl-1-nitro-2-(trifluoromethoxy)benzene

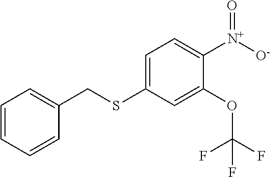 [SMILES: O=[N+]([O-])c1ccc(SCc2ccccc2)cc1OC(F)(F)F]

In a sealed tube, a solution of 4-chloro-1-nitro-2-(trifluoromethoxy)benzene (500 mg, 2.07 mmol) and DIPEA (723 μL, 4.14 mmol) in 1,4-dioxane (17 mL) was degassed with argon for 5 min, 9,9-dimethyl-4,5-bis(diphenylphosphino)xanthene (Xantphos) (60 mg, 0.103 mmol), tris(dibenzylideneacetone)dipalladium(0) (190 mg, 0.207 mmol) and benzyl mercaptan (514 mg, 4.14 mmol) were added and the mixture was degassed with argon for 5 min. The mixture was stirred at 100° C. for 16 h. The mixture was allowed to cool to rt and was poured into half saturated aqueous NaHCO₃. The aqueous layer was extracted twice with EtOAc. The combined organic layers were washed with brine, dried over sodium sulfate, filtered, and concentrated. The crude was purified by flash chromatography on silica gel using a gradient of EtOAc in heptane from 2% to 10% to afford the title compound as a yellow oil. (380 mg, 47% yield, tᵣ=1.05 min). LCMS (Method F); ¹H-NMR (DMSO-d₆, 400 MHz) δ (ppm) 8.11 (d, J=8.7 Hz, 1H), 7.63-7.52 (m, 2H), 7.50-7.40 (m, 2H), 7.39-7.31 (m, 2H), 7.31-7.23 (m, 1H), 4.49 (s, 2H).

### Step 2: Synthesis of N-(1-(3,4-dichlorophenyl)-2-(dimethylamino)ethyl)-4-nitro-3-(trifluoromethoxy)benzenesulfonamide (222)

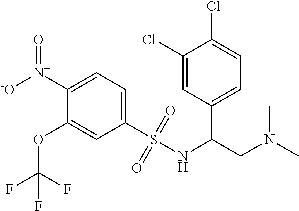

In a sealed tube, a solution of 4-benzylsulfanyl-1-nitro-2-(trifluoromethoxy)benzene (100 mg, 0.304 mmol) in acetonitrile (3 mL), water (76 μL) and acetic acid (38 μL) was stirred at 0° C. 1,3-dichloro-5,5-dimethyl-imidazolidine-2,4-dione (120 mg, 0.607 mmol) was added and the mixture was stirred at 0° C. for 3 h. 1-(3,4-dichlorophenyl)-N²,N²-dimethylethane-1,2-diamine (106 mg, 0.456 mmol) was added dropwise then a solution of triethylamine (47 μL, 0.334 mmol) in acetonitrile (300 μL) was added. The mixture was stirred at rt for 16 h and was diluted with half saturated aqueous NaHCO₃. The mixture was extracted three times with DCM, the combined organic layers were washed with brine, dried over sodium sulfate, filtered and concentrated. The crude was purified by flash chromatography on silica gel using a gradient of (MeOH+2% NH₄OH) in DCM from 0.2% to 10%. The desired fractions were concentrated and the residue was triturated in pentanes/Et₂O (95/5), the obtained suspension was filtered, the residue was washed with pentane and dried under reduced pressure at 45° C. for 16 h to afford the title compound as a white powder (16 mg, 10% yield, 95.3% purity, tᵣ=1.49 min). LCMS (Method C): m/z found 503 [M+H]⁺; ¹H-NMR (500 MHz, DMSO-d₆) δ (ppm) 8.99-8.63 (m, 1H), 8.24 (d, J=8.6 Hz, 1H), 7.85 (dd, J=8.6, 1.7 Hz, 1H), 7.76 (s, 1H), 7.42 (d, J=8.3 Hz, 1H), 7.39 (d, J=2.0 Hz, 1H), 7.17 (dd, J=8.3, 2.0 Hz, 1H), 4.47 (br dd, J=8.4, 6.5 Hz, 1H), 2.54-2.45 (m, 1H), 2.28 (dd, J=12.6, 6.0 Hz, 1H), 2.06 (s, 6H).

### Example 100: N-(1-(3,4-dichlorophenyl)-2-(dimethylamino)ethyl)-2-nitro-3-(trifluoromethoxy)benzenesulfonamide (223)

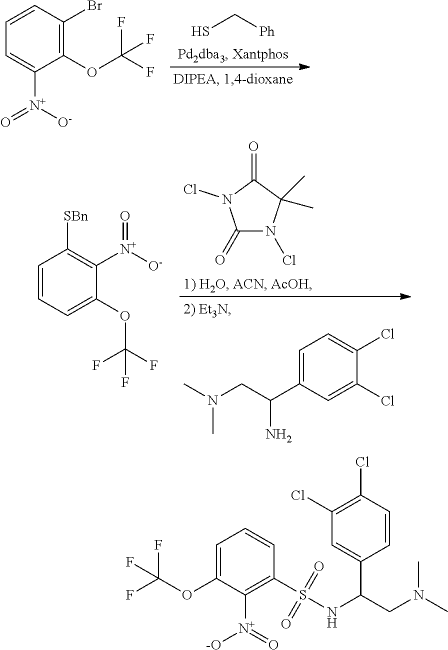

### Step 1: Synthesis of 1-benzylsulfanyl-2-nitro-3-(trifluoromethoxy)benzene

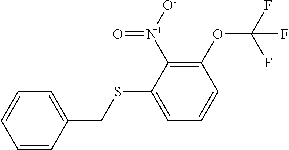 [SMILES: O=[N+]([O-])c1c(OC(F)(F)F)cccc1SCc1ccccc1]

A sealed vial was charged with 1-bromo-2-nitro-3-(trifluoromethoxy)benzene (98%, 300 mg, 1.03 mmol), and DIPEA (359 μL, 2.06 mmol) in anhydrous 1,4-dioxane (10.28 mL). The reaction mixture was degassed with argon for 5 min. Next, Xantphos (59 mg, 0.103 mmol), tris(dibenzylideneacetone)dipalladium(0) (47 mg, 0.0514 mmol), and phenyl mercaptan (241 μL, 2.06 mmol) were added, and the reaction mixture was stirred at 105° C. overnight. The mixture was cooled to rt, filtered on a pad of dicalite, washed with dioxane and DCM, and purified by flash chromatography on silica gel using a gradient of EtOAc in heptane from 0% to 10% to afford the title compound as a yellow oil (259.2 mg, 77% yield, tᵣ=1.04 min). LCMS (Method F); ¹H-NMR (DMSO-d₆, 400 MHz) δ (ppm) 7.79 (dd, J=8.2, 1.1 Hz, 1H), 7.71 (t, J=8.2 Hz, 1H), 7.60-7.52 (m, 1H), 7.37-7.20 (m, 5H), 4.41 (s, 2H).

### Step 2: Synthesis of N-(1-(3,4-dichlorophenyl)-2-(dimethylamino)ethyl)-2-nitro-3-(trifluoromethoxy)benzenesulfonamide (223)

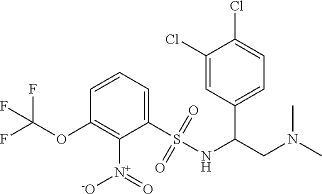

In a round-bottomed flask, to a stirred solution of 1-benzylsulfanyl-2-nitro-3-(trifluoromethoxy)benzene (511 mg, 1.55 mmol) in acetonitrile (13 mL) at 0° C. under nitrogen were added successively water (0.33 mL), acetic acid (0.5 mL) and then 1,3-dichloro-5,5-dimethyl-imidazolidine-2,4-dione (611 mg, 3.10 mmol). The reaction mixture was stirred at 0° C. for 2 h, then 3 mL were taken, concentrated in vacuo, dissolved in DCM and quenched with 10 mL NaHCO₃ (5%) at 0° C. The organic layer was passed through a phase separator and added dropwise to a solution of and triethylamine (0.087 mL, 0.621 mmol) in DCM, and stirred overnight at rt. A half saturated solution of NaHCO₃ was added, and the mixture was extracted twice with DCM. The combined organic layers were passed through a phase separator, concentrated in vacuo, and purified by flash chromatography on silica gel using a gradient of methanol (0.7 N NH₃) in DCM from 0.4% to 2%. Desired fractions were combined, dissolved in diethyl ether, pentane was added and the product slowly crystallized. The solid was filtered, washed with pentane and dried under vacuum overnight to afford the title compound as a white powder (31 mg, 4% yield, 98.9% purity, tᵣ=1.51 min). LCMS (Method C): m/z found 502 [M+H]⁺; ¹H-NMR (DMSO-d₆, 500 MHz): δ (ppm) 8.41-9.21 (m, 1H), 7.94 (dd, J=7.9, 1.1 Hz, 1H), 7.91 (dt, J=8.4, 1.3 Hz, 1H), 7.78-7.82 (m, 1H), 7.47 (d, J=2.0 Hz, 1H), 7.44 (d, J=8.3 Hz, 1H), 7.23 (dd, J=8.3, 2.2 Hz, 1H), 4.43 (dd, J=8.9, 6.0 Hz, 1H), 2.52-2.56 (m, 1H), 2.30-2.35 (m, 1H), 2.07 (s, 6H).

### Example 101: tert-butyl (2-(3,4-dichlorophenyl)-2-((4-(trifluoromethoxy)phenyl)sulfonamido)ethyl)carbamate (224)

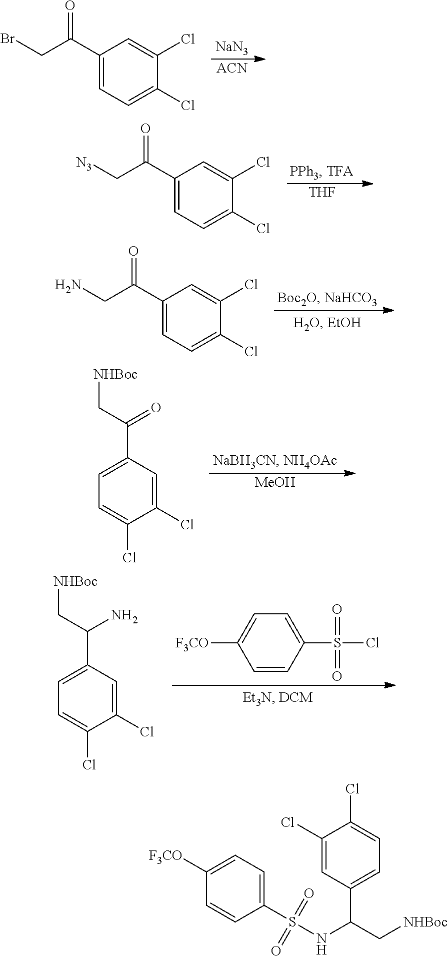

### Step 1: Synthesis of 2-azido-1-(3,4-dichlorophenyl)ethanone

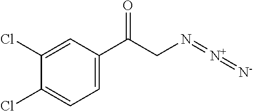 [SMILES: [N-]=[N+]=NCC(=O)c1ccc(Cl)c(Cl)c1]

In a round bottomed flask under nitrogen, a solution of 2-bromo-1-(3,4-dichlorophenyl)ethanone (300 mg, 1.12 mmol) in acetonitrile (4.1237 mL) was stirred at 0° C. NaN₃ (218 mg, 3.36 mmol) was added portion-wise, and the mixture was stirred at 0° C. for 15 min, then at rt for 22 h. The mixture was filtered, the residue was washed with Et₂O, and the filtrate was concentrated to afford the title compound as an orange solid (260 mg, 99% yield, tᵣ=0.92 min). LCMS (Method E); ¹H-NMR (DMSO-d₆, 400 MHz) δ (ppm) 8.16 (d, J=1.9 Hz, 1H), 7.90 (dd, J=8.4, 2.0 Hz, 1H), 7.86 (d, J=8.4 Hz, 1H), 4.91 (s, 2H).

### Step 2: Synthesis of 2-amino-1-(3,4-dichlorophenyl)ethenone

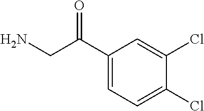 [SMILES: NCC(=O)c1ccc(Cl)c(Cl)c1]

In a round bottomed flask under nitrogen, triphenylphosphine (293 mg, 1.12 mmol) then 4-methylbenzenesulfonic acid (578 mg, 3.36 mmol) were added to a stirred solution of 2-azido-1-(3,4-dichlorophenyl)ethanone (99%, 260 mg, 1.12 mmol) in THF (5 mL). The mixture was stirred at rt for 1.5 h. The obtained suspension was filtered, the residue washed with THF, and dried under reduced pressure at 45° C. for 2 h to afford the title compound as a white powder (305 mg, 72% yield, tᵣ=0.59 min). LCMS (Method E): m/z found 204.1 [M+H]⁺; ¹H-NMR (DMSO-d₆, 400 MHz) δ (ppm) 8.34-8.11 (m, 4H), 7.98 (dd, J=8.4, 2.0 Hz, 1H), 7.90 (d, J=8.4 Hz, 1H), 7.55-7.40 (m, 2H), 7.11 (d, J=7.8 Hz, 2H), 4.64 (s, 2H), 2.29 (s, 3H).

### Step 3: Synthesis of tert-butyl N-[2-amino-2-(3,4-dichlorophenyl)ethyl]carbamate

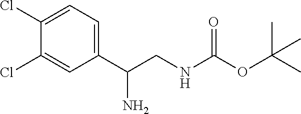 [SMILES: CC(C)(C)OC(=O)NCC(N)c1ccc(Cl)c(Cl)c1]

In a round bottomed flask, sodium hydrogen carbonate (170 mg, 2.03 mmol) was added portion-wise to a stirred suspension of 2-amino-1-(3,4-dichlorophenyl)ethanone (305 mg, 0.811 mmol) and di-tert-butyl dicarbonate (265 mg, 1.22 mmol) in methanol (4 mL) and water (4 mL). The suspension was stirred at rt for 16 h and was poured into 20 mL of cold water. The suspension was filtered, the residue was washed with water, then dissolved in DCM. The organic layer was dried over sodium sulfate, filtered, and concentrated to afford the title compound as a white solid (190 mg, 75% yield, tᵣ=0.96 min). LCMS (Method E): m/z found [M-Boc+H]⁺; ¹H-NMR (DMSO-d₆, 400 MHz) δ (ppm) 8.18 (d, J=1.9 Hz, 1H), 7.93 (dd, J=8.4, 2.0 Hz, 1H), 7.82 (d, J=8.4 Hz, 1H), 7.15 (t, J=5.7 Hz, 1H), 4.44 (d, J=5.9 Hz, 2H), 1.39 (s, 9H).

### Step 4: Synthesis of tert-butyl N-[2-amino-2-(3,4-dichlorophenyl)ethyl]carbamate

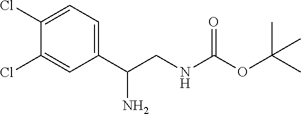 [SMILES: CC(C)(C)OC(=O)NCC(N)c1ccc(Cl)c(Cl)c1]

In a sealed tube under nitrogen supported NaBH₃CN (729 mg, 11.6 mmol) was added to a stirred solution of tert-butyl N-[2-(3,4-dichlorophenyl)-2-oxo-ethyl]carbamate (176 mg, 0.580 mmol) and ammonium acetate (894 mg, 11.6 mmol) in methanol (20 mL). The mixture was stirred at 65° C. for 16 h, then concentrated, and the residue was dissolved in DCM and saturated aqueous NaHCO₃. The aqueous layer was extracted twice with DCM. The combined organic layers were washed with brine, dried over sodium sulfate, filtered and concentrated. The crude product was purified by flash chromatography on silica gel using a gradient of (MeOH+2% NH₄OH) in DCM from 1% to 5% to afford the title compound as colorless oil (110 mg, 62% yield, tᵣ=0.62 min). LCMS (Method E): m/z found 305.1 [M+H]⁺; ¹H-NMR (DMSO-d₆, 400 MHz) δ (ppm) 7.67-7.41 (m, 2H), 7.30 (d, J=8.1 Hz, 1H), 6.80 (s, 1H), 3.88 (t, J=6.2 Hz, 1H), 3.17-2.87 (m, 2H), 2.30-1.72 (m, 2H), 1.33 (s, 9H).

### Step 5: Synthesis of tert-butyl N-[2-(3,4-dichlorophenyl)-2-[[4-(trifluoromethoxy)phenyl]sulfonylamino]ethyl]carbamate (224)

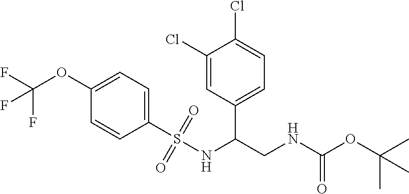 [SMILES: CC(C)(C)OC(=O)NCC(NS(=O)(=O)c1ccc(OC(F)(F)F)cc1)c1ccc(Cl)c(Cl)c1]

In a sealed tube under nitrogen, triethylamine (201 μL, 1.44 mmol) was added to a stirred solution of 4-(trifluoromethoxy)benzenesulfonyl chloride (98%, 96 mg, 0.360 mmol) and tert-butyl N-[2-amino-2-(3,4-dichlorophenyl)ethyl]carbamate (110 mg, 0.360 mmol) in DCM (2 mL). The solution was stirred at rt for 16 h and was poured into half saturated aqueous NaHCO₃. The aqueous layer was extracted three times with DCM. The combined organic layers were washed with brine, dried over sodium sulfate, filtered and concentrated. The crude was purified by flash chromatography on silica gel using a gradient of (MeOH+2% NH₄OH) in DCM from 1% to 5% to afford the title compound as a white powder. (133 mg, 69% yield, 99.3% purity, tᵣ=2.86 min). LCMS (Method C): m/z found 529.36 [M+H]⁺; ¹H-NMR (DMSO-d₆, 500 MHz): δ (ppm) 8.48 (s, 1H), 7.65 (d, J=8.8 Hz, 2H), 7.34 (dd, J=8.2, 4.0 Hz, 3H), 7.22 (d, J=1.5 Hz, 1H), 7.02 (dd, J=8.3, 1.7 Hz, 1H), 6.80 (br t, J=5.9 Hz, 1H), 4.39 (br t, J=7.0 Hz, 1H), 3.04-3.21 (m, 2H), 1.27 (s, 9H).

### Example 102: N-(2-amino-1-(3,4-dichlorophenyl)ethyl)-4-(trifluoromethoxy)benzenesulfonamide hydrochloride (225)

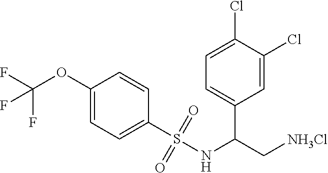

In a sealed tube under nitrogen, a solution of tert-butyl N-[2-(3,4-dichlorophenyl)-2-[[4-(trifluoromethoxy)phenyl]sulfonylamino]ethyl]carbamate (30 mg, 0.0567 mmol) in 4 M hydrogen chloride in dioxane (425 μL, 1.70 mmol) was stirred at rt for 4 h. The mixture was diluted with Et₂O (2 mL) and was filtered. The residue was washed with Et₂O and dried under reduced pressure at 45° C. for 16 h to afford the title product as a white powder. (24 mg, 90% yield, 100% purity, tᵣ=1.42 min). LCMS (Method C): m/z found 429 [M+H]⁺; 1H-NMR (500 MHz, DMSO-d₆) δ (ppm) 8.65-7.70 (m, 4H), 7.63 (d, J=8.8 Hz, 2H), 7.38 (d, J=8.3 Hz, 1H), 7.35-7.28 (m, 3H), 7.10 (dd, J=8.3, 2.0 Hz, 1H), 4.60 (dd, J=8.4, 6.2 Hz, 1H), 3.18-2.87 (m, 2H).

### Example 103: methyl 5-(N-(1-(3,4-dichlorophenyl)-2-(dimethylamino)ethyl)sulfamoyl)-2-(trifluoromethoxy)benzoate (226)

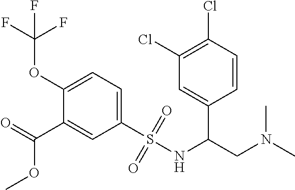 [SMILES: COC(=O)c1cc(S(=O)(=O)NC(CN(C)C)c2ccc(Cl)c(Cl)c2)ccc1OC(F)(F)F]

In a sealed tube, methyl 5-(chlorosulfonyl)-2-(trifluoromethoxy)benzoate (95%, 40 μL, 1.51 mmol) was added to a stirred solution of 1-(3,4-dichlorophenyl)-N′,N′-dimethyl-ethane-1,2-diamine (90%, 390 mg, 1.51 mmol) and triethylamine (630 μL, 4.52 mmol) in anhydrous DCM (14.04 mL). The solution was stirred at room temperature for 3 h. The reaction mixture was diluted with DCM. The organic layer was washed once with a saturated aqueous solution of NH₄Cl, once with a saturated solution of NaHCO₃, and once with a saturated aqueous solution of sodium chloride. The organic layer was passed through a phase separator and concentrated under reduced pressure. The crude was purified by flash chromatography on silica gel using a gradient of methanol in dichloromethane from 0.5% to 5%. The desired fractions were combined to afford a beige solid (672.2 mg, 87% yield). A sample (130 mg) of the beige solid was triturated in pentane and dried under vacuum at 45° C. for 48 h to afford the title compound as white powder (102.7 mg, 79% recovery, 99.7% purity, tᵣ=1.45 min). LCMS (Method C): m/z found 515.2 [M+H]⁺; ¹H-NMR (500 MHz, DMSO-d₆) δ (ppm) 8.87-8.27 (m, 1H), 8.01 (d, J=2.2 Hz, 1H), 7.93-7.88 (m, 1H), 7.61-7.53 (m, 1H), 7.39-7.35 (m, 1H), 7.35-7.31 (m, 1H), 7.15-7.08 (m, 1H), 4.50-4.35 (m, 1H), 3.91 (s, 3H), 2.48-2.22 (m, 2H), 2.05 (s, 5H), 2.00-1.94 (m, 1H).

### Example 104: N-((1-(3,4-dichlorophenyl)cyclopropyl)methyl)-4-(trifluoromethoxy)benzenesulfonamide (227)

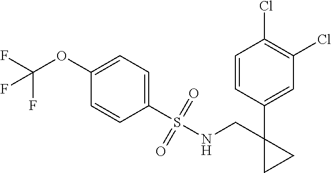 [SMILES: O=S(=O)(NCC1(c2ccc(Cl)c(Cl)c2)CC1)c1ccc(OC(F)(F)F)cc1]

In a sealed tube, 4-(trifluoromethoxy)benzenesulfonyl chloride (98%, 33 μL, 0.191 mmol) was added to a stirred solution of [1-(3,4-dichlorophenyl)cyclopropyl]methanamine (0.042 mL, 0.190 mmol) and triethylamine (79 μL, 0.569 mmol) in anhydrous DCM (1.4 mL). The solution was stirred at room temperature for 3 h. The reaction mixture was diluted with DCM. The organic layer was washed once with a saturated aqueous solution of NH₄Cl, once with a saturated solution of NaHCO₃ and once with a saturated aqueous solution of sodium chloride. The organic layer was dried trough a phase separator and concentrated under reduced pressure. The crude was purified by flash chromatography on silica gel using a gradient of EtOAc in heptane from 0% to 50%. The resulting oil was solubilized in Et₂O and the homogenous solution was added to pentane. The precipitate formed was filtered, washed with pentane and dried under vacuum at 45° C. for 2 days to afford the title compounds as white powder (33.6 mg, 40% yield, 98.8% purity, tᵣ=2.92 min). LCMS (Method C): m/z found 438.1 [M+H]⁺; ¹H-NMR (600 MHz, DMSO-d₆) δ (ppm) 7.92 (s, 1H), 7.76-7.85 (m, 2H), 7.50 (d, J=8.1 Hz, 2H), 7.46 (d, J=8.4 Hz, 1H), 7.44 (d, J=2.2 Hz, 1H), 7.19 (dd, J=8.4, 2.2 Hz, 1H), 2.99-3.08 (m, 2H), 0.66-0.94 (m, 4H).

### Example 105: N-((1-(4-chlorophenyl)cyclopropyl)methyl)-4-(trifluoromethoxy)benzenesulfonamide (228)

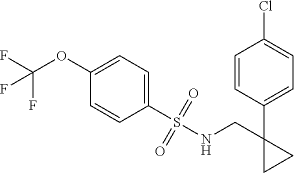 [SMILES: O=S(=O)(NCC1(c2ccc(Cl)cc2)CC1)c1ccc(OC(F)(F)F)cc1]

In a sealed tube, 4-(trifluoromethoxy)benzenesulfonyl chloride (98%, 46 μL, 0.267 mmol) was added to a stirred solution of 1-[1-(4-chlorophenyl)cyclopropyl]methanamine (97%, 0.042 mL, 0.267 mmol) and triethylamine (112 μL, 0.801 mmol) in anhydrous DCM (2 mL). The solution was stirred at room temperature for 3 h. The reaction mixture was diluted with DCM. The organic layer was washed once with a saturated aqueous solution of NH₄Cl, once with a saturated solution of NaHCO₃ and once with a saturated aqueous solution of sodium chloride. The organic layer was dried trough a phase separator and concentrated under reduced pressure. The crude was purified by flash chromatography on silica gel using a gradient of EtOAc in cyclohexane from 0% to 100%. The resulting solid was solubilized in DCM and the homogenous solution was added to pentane. The precipitate formed was filtered, washed with pentane and dried under vacuum at 45° C. for 2 days to afford the title compound as white powder (65.3 mg, 60% yield, 99.7% purity, tᵣ=2.78 min). LCMS (Method C): m/z found 406 [M+H]⁺; ¹H-NMR (500 MHz, DMSO-d₆) δ (ppm) 7.85-7.92 (m, 1H), 7.81 (d, J=8.5 Hz, 2H), 7.51 (d, J=8.5 Hz, 2H), 7.25-7.29 (m, 2H), 7.22-7.25 (m, 2H), 3.01 (s, 2H), 0.56-0.89 (m, 4H).

### Example 106: N-((1-(4-chlorophenyl)cyclobutyl)methyl)-4-(trifluoromethoxy)benzenesulfonamide (229)

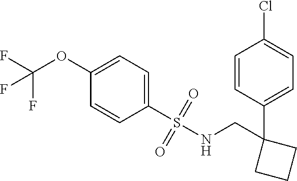 [SMILES: O=S(=O)(NCC1(c2ccc(Cl)cc2)CCC1)c1ccc(OC(F)(F)F)cc1]

### Step 1: Synthesis of 1-(4-chlorophenyl)cyclobutane-1-carbonitrile

In a three-neck round-bottomed flask, to a stirred suspension of sodium hydride (60%, 689 mg, 17.2 mmol) in anhydrous DMF (16 mL) at 0° C. under nitrogen was added dropwise a solution of (4-chlorophenyl)acetonitrile (95%, 1.00 g, 6.27 mmol) in anhydrous DMF (6 mL). The reaction mixture was stirred at 0° C. for 15 min (until no gas production was observed). A solution of 1,3-dibromopropane (954 μL, 9.40 mmol) in anhydrous DMF (6 mL) was added dropwise at 0° C. and then the reaction mixture was allowed to warm up to room temperature and stirred for 3 h. The reaction mixture was quenched with a saturated aqueous solution of ammonium chloride (50 mL) and ethyl acetate (75 mL) and water (40 mL) were added. The aqueous layer was extracted with ethyl acetate (1×50 mL). The combined organic layers were washed with water, a saturated aqueous solution of sodium chloride, dried over sodium sulfate, filtered and concentrated under reduced pressure. The crude was purified by flash chromatography on silica gel using a gradient of ethyl acetate in cyclohexane from 0% to 40% to afford the title compound as a colorless oil (990 mg, 82% yield, tᵣ=0.91 min). LCMS (Method F); ¹H-NMR (DMSO-d₆, 400 MHz) δ (ppm) 7.77-7.24 (m, 4H), 2.79-2.70 (m, 2H), 2.66-2.56 (m, 2H), 2.28 (dp, J=11.3, 8.7 Hz, 1H), 2.01 (dtt, J=11.3, 8.9, 4.4 Hz, 1H).

### Step 2: Synthesis of 1-[1-(4-chlorophenyl)cyclobutyl]methanamine

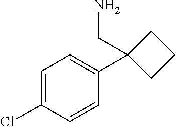 [SMILES: NCC1(c2ccc(Cl)cc2)CCC1]

In a round-bottomed flask, to a stirred solution of 1-(4-chlorophenyl)cyclobutanecarbonitrile (985 mg, 5.14 mmol) in anhydrous THF (22 mL) at 0° C. under nitrogen was added 2.4 M LAH in THF (5.4 mL, 12.8 mmol). The reaction mixture was stirred at room temperature for 2 h. The reaction mixture was quenched at 0° C. with water (490 μL, same weight as LAH used). Next, 1 M sodium hydroxide (516 μL, 0.516 mmol) and water (three times the weight of LAH used) were added. The resulting heterogenous mixture was stirred at room temperature for 15 min, dried over sodium sulfate, filtered, rinsed with THF, and concentrated under reduced pressure. The crude was purified by flash chromatography on silica gel using a gradient of ammoniacal methanol in dichloromethane from 3% to 10% to afford the title compound as a yellow oil (715 mg, 71% yield, tᵣ=0.84 min). LCMS (Method F): m/z found 196.2 [M+H]⁺; ¹H-NMR (DMSO-d₆, 400 MHz) δ (ppm) 7.40-7.29 (m, 2H), 7.16-7.04 (m, 2H), 2.75 (s, 2H), 2.20-2.08 (m, 4H), 2.05-1.91 (m, 1H), 1.83-1.71 (m, 1H), 1.11 (s, 2H).

### Step 3: Synthesis of N-((1-(4-chlorophenyl)cyclobutyl)methyl)-4-(trifluoromethoxy)benzenesulfonamide (229)

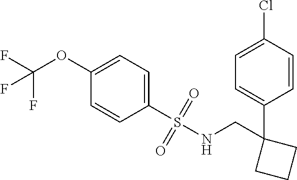 [SMILES: O=S(=O)(NCC1(c2ccc(Cl)cc2)CCC1)c1ccc(OC(F)(F)F)cc1]

In a sealed tube, 4-(trifluoromethoxy)benzenesulfonyl chloride (67 μL, 0.393 mmol) was added to a stirred solution of 1-[1-(4-chlorophenyl)cyclobutyl]methanamine (70 mg, 0.358 mmol) and triethylamine (150 μL, 1.07 mmol) in DCM (3.3 mL). The solution was stirred at room temperature for 3 h. Water (5 mL) was added and the aqueous layer was extracted with dichloromethane (1×10 mL). The combined organic layers were washed with a saturated aqueous solution of sodium chloride, dried over sodium sulfate, filtered and concentrated under reduced pressure. The crude was purified by flash chromatography on silica gel using a gradient of ethyl acetate in cyclohexane from 0% to 30% to afford the title compound as a colorless oil (137.8 mg, 91% yield, 99.4% purity, tᵣ=2.97 min). LCMS (Method C): m/z found 420.2 [M+H]⁺; ¹H-NMR (500 MHz, DMSO-d₆) δ (ppm) 7.83-7.79 (m, 2H), 7.68 (s, 1H), 7.51 (d, J=8.1 Hz, 2H), 7.31-7.28 (m, 2H), 7.10-7.07 (m, 2H), 3.05 (s, 2H), 2.24-2.12 (m, 4H), 2.00-1.90 (m, 1H), 1.77-1.69 (m, 1H).

### Example 107: N-((2,2-dimethyl-1-phenylcyclopropyl)methyl)-4-(trifluoromethoxy)benzenesulfonamide (230)

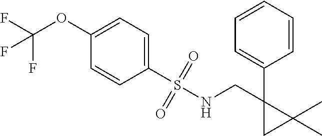 [SMILES: CC1(C)CC1(CNS(=O)(=O)c1ccc(OC(F)(F)F)cc1)c1ccccc1]

In a sealed tube, 4-(trifluoromethoxy)benzenesulfonyl chloride (71 μL, 0.417 mmol) was added to a stirred solution of 1-(2,2-dimethyl-1-phenylcyclopropyl)methanamine (95%, 70 mg, 0.379 mmol) and triethylamine (159 μL, 1.14 mmol) in DCM (3.5623 mL). The solution was stirred at room temperature for 3 h. Water (5 mL) was added and the aqueous layer was extracted with dichloromethane (1×10 mL). The combined organic layers were washed with a saturated aqueous solution of sodium chloride, dried over sodium sulfate, filtered and concentrated under reduced pressure. The crude was purified by flash chromatography on silica gel using a gradient of ethyl acetate in cyclohexane from 0% to 30%. The residue was triturated in pentane, filtered, washed with pentane and dried under vacuum at 70° C. for 18 h to afford the title compound as a white powder (114.6 mg, 75% yield, 99.7% purity, tᵣ=2.92 min). LCMS (Method C): m/z found 400.2 [M+H]⁺; ¹H-NMR (500 MHz, DMSO-d₆) δ (ppm) 7.72 (d, J=8.5 Hz, 2H), 7.54-7.66 (m, 1H), 7.46 (d, J=8.5 Hz, 2H), 7.07-7.26 (m, 5H), 2.95-3.24 (m, 2H), 1.15 (s, 3H), 0.63-0.82 (m, 2H), 0.62 (s, 3H).

### Example 108: N-((1-(3,4-dichlorophenyl)cyclopropyl)methyl)-4-(4-(trifluoromethyl)phenoxy)benzenesulfonamide (231)

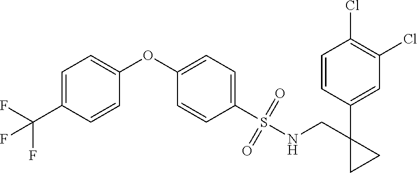 [SMILES: O=S(=O)(NCC1(c2ccc(Cl)c(Cl)c2)CC1)c1ccc(Oc2ccc(C(F)(F)F)cc2)cc1]

In a sealed tube, 4-[4-(trifluoromethyl)phenoxy]benzenesulfonyl chloride (67 mg, 0.200 mmol) was added to a stirred solution of [1-(3,4-dichlorophenyl)cyclopropyl]methanamine (0.042 mL, 0.200 mmol) and triethylamine (84 μL, 0.600 mmol) in anhydrous DCM (1.5 mL). The solution was stirred at room temperature for 3 h. The reaction mixture was diluted with DCM. The organic layer was washed once with a saturated aqueous solution of NH₄Cl, once with a saturated solution of NaHCO₃ and once with a saturated aqueous solution of sodium chloride. The organic layer was dried trough a phase separator and concentrated under reduced pressure. The crude was purified by flash chromatography on silica gel using a gradient of EtOAc in heptane from 0% to 50%. The resulting solid was solubilized in Et₂O and the homogenous solution was added to pentane. The precipitate formed was filtered and dried under vacuum at 45° C. for 2 days to afford the title compound as white powder (41.5 mg, 40% yield, 98.6% purity, tᵣ=3.22 min). LCMS (Method C): m/z found 514.2 [M−H]⁻; ¹H-NMR (600 MHz, DMSO-d₆, 300K) δ (ppm) 7.81 (d, J=8.5 Hz, 2H), 7.78 (s, 1H), 7.72 (d, J=8.9 Hz, 2H), 7.49 (d, J=8.4 Hz, 1H), 7.46 (d, J=2.2 Hz, 1H), 7.28 (d, J=8.5 Hz, 2H), 7.22 (dd, J=8.4, 2.2 Hz, 1H), 7.18 (d, J=8.9 Hz, 2H), 2.94-3.10 (m, 2H), 0.73-0.88 (m, 4H).

### Example 109: N-((1-(3,4-dichlorophenyl)cyclopropyl)methyl)-3-nitro-4-(trifluoromethoxy)benzenesulfonamide (232)

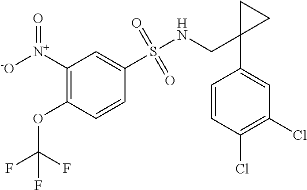

In a sealed tube, 3-nitro-4-(trifluoromethoxy)benzenesulfonyl chloride (36 L, 0.193 mmol) was added to a stirred solution of [1-(3,4-dichlorophenyl)cyclopropyl]methanamine (0.042 mL, 0.193 mmol) and triethylamine (81 μL, 0.579 mmol) in Anhydrous DCM (1.5 mL). The solution was stirred at room temperature for 3 h. The reaction mixture was diluted with DCM. The organic layer was washed once with a saturated aqueous solution of NH₄Cl, once with a saturated solution of NaHCO₃, and once with a saturated aqueous solution of sodium chloride. The organic layer was dried trough a phase separator and concentrated under reduced pressure. The crude was purified by flash chromatography on silica gel using a gradient of EtOAc in heptane from 0% to 50%. The resulting solid was solubilized in Et₂O and the homogenous solution was added to pentane. The precipitate formed was filtered, washed with pentane and dried under vacuum at 45° C. for 2 days to afford the title compound as white powder (45.2 mg, 44% yield, 91.4% purity, tᵣ=2.89 min). LCMS (Method C): m/z found 483.1 [M−H]⁻; ¹H-NMR (600 MHz, DMSO-d₆, 300K) δ (ppm) 8.38 (d, J=2.3 Hz, 1H), 8.23 (s, 1H), 8.08 (dd, J=8.7, 2.3 Hz, 1H), 7.84-7.91 (m, 1H), 7.36-7.54 (m, 2H), 7.19 (dd, J=8.2, 2.1 Hz, 1H), 3.14 (s, 2H), 0.67-0.98 (m, 4H).

### Example 110: N-(1-(3,4-dichlorophenyl)-2-(4-methylpiperazin-1-yl)ethyl)-4-(4-(trifluoromethyl)phenoxy)benzenesulfonamide (233)

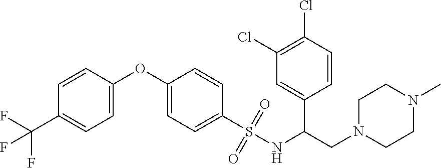 [SMILES: CN1CCN(CC(NS(=O)(=O)c2ccc(Oc3ccc(C(F)(F)F)cc3)cc2)c2ccc(Cl)c(Cl)c2)CC1]

In a sealed tube, 4-[4-(trifluoromethyl)phenoxy]benzenesulfonyl chloride (42 μL, 0.191 mmol) was added to a stirred solution of 1-(3,4-dichlorophenyl)-2-(4-methylpiperazin-1-yl)ethanamine (55 mg, 0.191 mmol) and triethylamine (80 μL, 0.572 mmol) in anhydrous DCM (2.2 mL). The solution was stirred at room temperature for 3 h. Water (5 mL) was added and the aqueous layer was extracted with dichloromethane (2×7 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was triturated in 2 M hydrogen chloride in diethyl ether (0.95 mL, 1.91 mmol), filtered, washed with diethyl ether. The crude was purified by flash chromatography on silica gel using a gradient of ammoniacal methanol in dichloromethane from 3% to 10%. The residue was triturated in diethyl ether, filtered, washed with diethyl ether and dried under vacuum at 70° C. for 20 h to afford the title compound as a white powder (45.2 mg, 37% yield, 98.6% purity, tᵣ=1.92 min). LCMS (Method C): m/z found 588 [M+H]⁺; ¹H-NMR (DMSO-d₆, 500 MHz): δ (ppm) 9.80 (br s, 1H), 8.27 (d, J=8.3 Hz, 1H), 7.83 (d, J=8.6 Hz, 2H), 7.61-7.65 (m, J=8.8 Hz, 2H), 7.47 (d, J=8.1 Hz, 1H), 7.42 (s, 1H), 7.18-7.23 (m, 3H), 7.05-7.09 (m, J=8.8 Hz, 2H), 4.39-4.49 (m, 1H), 3.20-3.30 (m, 2H), 2.75-3.04 (m, 4H), 2.71 (br s, 3H), 2.58 (dd, J=13.0, 8.6 Hz, 1H), 2.44-2.49 (m, 1H), 2.30-2.43 (m, 2H).

### Example 111: N-(1-(4-chlorophenyl)-2-(4-methylpiperazin-1-yl)ethyl)-4-phenoxybenzenesulfonamide (234)

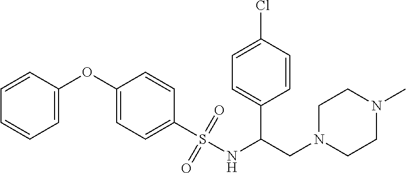 [SMILES: CN1CCN(CC(NS(=O)(=O)c2ccc(Oc3ccccc3)cc2)c2ccc(Cl)cc2)CC1]

In a sealed tube under nitrogen, triethylamine (110 μL, 0.788 mmol) was added to a stirred solution of 4-phenoxybenzenesulfonyl chloride (53 mg, 0.197 mmol) and 1-(4-chlorophenyl)-2-(4-methylpiperazin-1-yl)ethanamine (50 mg, 0.197 mmol) in DCM (1 mL). The solution was stirred at rt for 16 h and was poured into half saturated aqueous NaHCO₃. The aqueous layer was extracted three times with DCM. The combined organic layers were washed with brine, dried over sodium sulfate, filtered and concentrated. The crude was purified by flash chromatography on silica gel using a gradient of (MeOH+2% NH₄OH) in DCM from 1% to 10%. The desired fractions were triturated in a mixture of pentane/Et₂O (9/1). The obtained suspension was filtered, the residue was washed with pentane and dried under reduced pressure at 45° C. for 16 h to afford the title compound as a white powder. (69 mg, 71% yield, 99.8% purity, tᵣ=1.59 min). LCMS (Method C): m/z found 486.3 [M+H]⁺; ¹H-NMR (600 MHz, DMSO-d₆) δ 7.99 (br d, 1H, J=7.0 Hz), 7.62 (d, 2H, J=9.0 Hz), 7.4-7.5 (m, 2H), 7.2-7.3 (m, 5H), 7.0-7.1 (m, 2H), 6.94 (d, 2H, J=8.8 Hz), 4.3-4.4 (m, 1H), 3.38 (d, 1H, J=7.0 Hz), 3.30 (s, 6H), 2.5-2.5 (m, 9H), 1.9-2.5 (m, 13H), 1.2-1.3 (m, 1H), 1.09 (s, 1H), 0.8-0.9 (m, 1H).

### Example 112: N-(1-(4-chlorophenyl)-2-(4-methylpiperazin-1-yl)ethyl)-4-(4-(trifluoromethyl)phenoxy)benzenesulfonamide (235)

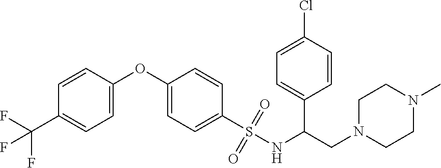 [SMILES: CN1CCN(CC(NS(=O)(=O)c2ccc(Oc3ccc(C(F)(F)F)cc3)cc2)c2ccc(Cl)cc2)CC1]

In a sealed tube under nitrogen, triethylamine (110 μL, 0.788 mmol) was added to a stirred solution of 1-(4-chlorophenyl)-2-(4-methylpiperazin-1-yl)ethanamine (50 mg, 0.197 mmol) and triethylamine (110 μL, 0.788 mmol) in DCM (1 mL). The solution was stirred at rt for 16 h and was poured into half saturated aqueous NaHCO₃. The aqueous layer was extracted three times with DCM. The combined organic layers were washed with brine, dried over sodium sulfate, filtered and concentrated. The crude was purified by flash chromatography on silica gel using a gradient of (MeOH+2% NH₄OH) in DCM from 1% to 10%. The desired fractions were dissolved in a mixture of pentane/Et₂O (9/1) and stirred at rt for 10 min. The obtained suspension was filtered, the residue was washed with pentane and dried under reduced pressure at 45° C. for 16 h to afford the title compound as a white powder (83 mg, 75% yield, 99.8% purity, tᵣ=1.82 min). LCMS (Method C): m/z found 554.3 [M+H]⁺; ¹H-NMR (600 MHz, DMSO-d₆) δ (ppm) 8.17-8.05 (m, 1H), 7.82 (d, J=8.5 Hz, 2H), 7.66 (d, J=8.8 Hz, 2H), 7.26 (d, J=8.2 Hz, 2H), 7.23-7.15 (m, 4H), 7.08 (d, J=8.8 Hz, 2H), 4.42-4.30 (m, 1H), 2.49-2.45 (m, 1H), 2.44-1.99 (m, 12H).

### Example 113: N-(1-(3,4-dichlorophenyl)-2-(4-methylpiperazin-1-yl)ethyl)-4-phenoxybenzenesulfonamide (236)

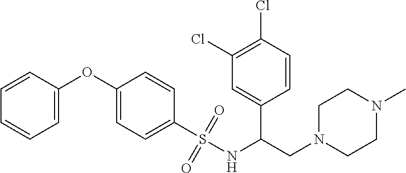 [SMILES: CN1CCN(CC(NS(=O)(=O)c2ccc(Oc3ccccc3)cc2)c2ccc(Cl)c(Cl)c2)CC1]

In a sealed tube, 4-phenoxybenzenesulphonyl chloride (42 μL, 0.191 mmol) was added to a stirred solution of 1-(3,4-dichlorophenyl)-2-(4-methylpiperazin-1-yl)ethanamine (55 mg, 0.191 mmol) and triethylamine (266 μL, 1.91 mmol) in anhydrous DCM (1.9773 mL). The solution was stirred at room temperature for 2 days. Water (5 mL) was added and the aqueous layer was extracted with dichloromethane (2×7 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The residue was triturated in 2 M hydrogen chloride in diethyl ether (0.95 mL, 1.91 mmol), filtered, washed with diethyl ether. The crude was purified by flash chromatography on silica gel using a gradient of ammoniacal methanol in dichloromethane from 3% to 10%. The residue was triturated in diethyl ether, filtered, washed with diethyl ether and dried under vacuum at 70° C. for 20 h to afford the title compound as a white powder (46 mg, 45.3% yield, 97.8% purity, tᵣ=1.7 min). LCMS (Method C): m/z found 520 [M+H]⁺; ¹H-NMR (500 MHz, DMSO-d₆) δ (ppm) 8.05 (br d, J=7.8 Hz, 1H), 7.60 (d, J=8.8 Hz, 2H), 7.52-7.43 (m, 3H), 7.40 (d, J=2.0 Hz, 1H), 7.26 (t, J=7.5 Hz, 1H), 7.19 (dd, J=8.3, 1.7 Hz, 1H), 7.05 (d, J=7.8 Hz, 2H), 6.92 (d, J=8.8 Hz, 2H), 4.42-4.31 (m, 1H), 2.48-1.97 (m, 13H).

### Example 114: N-(1-(3,4-dichlorophenyl)-2-(piperazin-1-yl)ethyl)-4-(trifluoromethoxy)benzenesulfonamide (237)

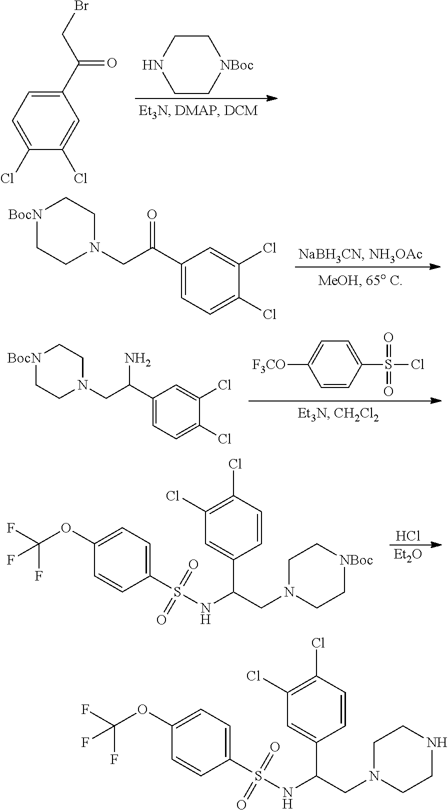

### Step 1: Synthesis of tert-butyl 4-[2-(3,4-dichlorophenyl)-2-oxo-ethyl]piperazine-1-carboxylate

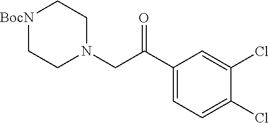 [SMILES: CC(C)(C)OC(=O)N1CCN(CC(=O)c2ccc(Cl)c(Cl)c2)CC1]

In a three necked round bottomed flask equipped with a thermometer, under nitrogen, a solution of 2-bromo-1-(3,4-dichlorophenyl)ethanone (10.00 g, 37.3 mmol), tert-butyl piperazine-1-carboxylate (12.51 g, 67.2 mmol) and DMAP (228 mg, 1.87 mmol) in DCM (100 mL) was stirred at rt. The mixture was stirred in an ice bath and triethylamine (5.7 mL, 41.1 mmol) was added. The cold bath was removed and the mixture was stirred at rt for 16 h. A half saturated aqueous solution of NaHCO₃ was added and the mixture was stirred at rt for 5 min. The aqueous layer was extracted twice with DCM. The combined organic layers were washed with brine, dried over sodium sulfate, filtered and concentrated. The crude was purified by flash chromatography on silica gel using a gradient of (MeOH+2% NH₄OH) in DCM from 0.4% to 4% to afford the title compound as a yellow solid (12.9 g, 91% yield, 99% purity, tᵣ=0.63). LCMS (Method F): m/z found 372.2 [M+H]⁺; ¹H-NMR (DMSO-d₆, 400 MHz): δ (ppm) 8.17 (d, J=2.0 Hz, 1H), 7.94 (dd, J=8.4, 2.0 Hz, 1H), 7.81 (d, J=8.4 Hz, 1H), 3.37-3.24 (m, 5H), 2.49-2.41 (m, 4H), 1.40 (s, 10H).

### Step 2: Synthesis of tert-butyl 4-[2-amino-2-(3,4-dichlorophenyl)ethyl]piperazine-1-carboxylate

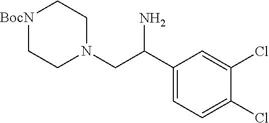 [SMILES: CC(C)(C)OC(=O)N1CCN(CC(N)c2ccc(Cl)c(Cl)c2)CC1]

In a sealed tube under nitrogen, polymer bound NaBH₃CN (34.40 g, 68.8 mmol) was added to a stirred solution of tert-butyl 4-[2-(3,4-dichlorophenyl)-2-oxo-ethyl]piperazine-1-carboxylate (12.84 g, 34.4 mmol) and ammonium acetate (98%, 27.06 g, 0.344 mol) in acetic acid (21.4 mL) and methanol (214 mL). The mixture was stirred at 40° C. for 16 h and was filtered. The residue was washed with MeOH and the filtrate was concentrated then dissolved in DCM and saturated aqueous NaHCO₃. The organic layer was washed with saturated aqueous NaHCO₃ then the combined aqueous layers were extracted with DCM. The combined organic layers were washed with brine, dried over sodium sulfate, filtered and concentrated. The crude was purified by flash chromatography on silica gel using a gradient of (MeOH+2% NH₄OH) in DCM from 1% to 5% to afford the title compound as a yellow solid. (6.9 g, 52% yield, 97% purity, tᵣ=0.65 min). LCMS (Method E): m/z found 374.3 [M+H]⁺; ¹H-NMR (DMSO-d₆, 400 MHz): δ (ppm) 7.65 (d, J=2.0 Hz, 1H), 7.55 (d, J=8.3 Hz, 1H), 7.36 (dd, J=8.3, 2.0 Hz, 1H), 4.05 (dd, J=8.6, 5.4 Hz, 1H), 3.41-3.18 (m, 7H), 2.48-2.37 (m, 2H), 2.37-2.22 (m, 4H), 1.99 (s, 2H), 1.39 (s, 10H).

### Step 3: Synthesis of tert-butyl 4-(2-(3,4-dichlorophenyl)-2-((4-(trifluoromethoxy)phenyl)sulfonamido)ethyl)piperazine-1-carboxylate

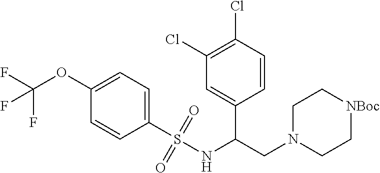 [SMILES: CC(C)(C)OC(=O)N1CCN(CC(NS(=O)(=O)c2ccc(OC(F)(F)F)cc2)c2ccc(Cl)c(Cl)c2)CC1]

In a sealed vial under nitrogen, 4-(trifluoromethoxy)benzenesulfonyl chloride (1.4 mL, 8.01 mmol) was added to a stirred solution of tert-butyl 4-[2-amino-2-(3,4-dichlorophenyl)ethyl]piperazine-1-carboxylate (3.00 g, 8.01 mmol) and triethylamine (4.5 mL, 32.1 mmol) in DCM (80 mL). The solution was stirred at rt for 16 h and was diluted with a half saturated aqueous solution of NaHCO₃ and DCM was added. The aqueous layer was extracted with DCM. The combined organic layers were washed with brine, dried over sodium sulfate, filtered and concentrated. The crude was purified by flash chromatography on silica gel using a gradient of (MeOH+2% NH₄OH) in DCM from 0.2% to 5% to afford the title compound as a white foam (4.0 g, 83% yield, 100% purity, tᵣ=0.80 min). LCMS (Method F): m/z found 598.46 [M+H]⁺; ¹H-NMR (DMSO-d₆, 400 MHz): δ (ppm) 8.34 (s, 1H), 7.84-7.74 (m, 2H), 7.51-7.39 (m, 4H), 7.22 (dd, J=8.3, 2.0 Hz, 1H), 4.42 (dd, J=8.8, 5.5 Hz, 1H), 3.20-2.94 (m, 4H), 2.50-2.45 (m, 1H), 2.38-2.10 (m, 5H), 1.37 (s, 9H).

### Step 4: Synthesis of N-(1-(3,4-dichlorophenyl)-2-(piperazin-1-yl)ethyl)-4-(trifluoromethoxy)benzenesulfonamide (237)

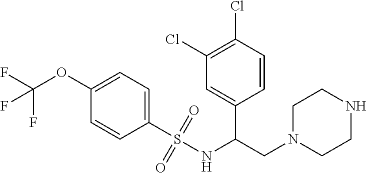 [SMILES: O=S(=O)(NC(CN1CCNCC1)c1ccc(Cl)c(Cl)c1)c1ccc(OC(F)(F)F)cc1]

In a sealed tube under nitrogen, 4 M hydrogen chloride in dioxane (67 mL, 0.267 mol) was added to a stirred solution of tert-butyl 4-[2-(3,4-dichlorophenyl)-2-[[4-(trifluoromethoxy)phenyl]sulfonylamino]ethyl]piperazine-1-carboxylate (4.00 g, 6.68 mmol) in diethyl ether (70 mL). The mixture was stirred at rt for 16 h, then diluted with Et₂O. The suspension was stirred at rt for 30 min, then filtered. The residue was washed with Et₂O, then dried under reduced pressure at 60° C. for 16 h. The solid was dissolved in refluxing MeOH (120 mL) and the solution was filtered. The filtrate was concentrated to about 20 mL and was stirred in an ice bath for 10 min. Et₂O (130 mL) was added portion-wise to the obtained suspension, then the suspension was stirred in an ice bath 20 min. The suspension was filtered, the residue was washed with Et₂O, then dried under reduced pressure at 60° C. for 16 h to afford the title compound as a white powder (2.91 g, 80% yield, 100% purity, tᵣ=1.58 min). LCMS (Method C): m/z found 534.81 [M+H]⁺; ¹H-NMR (DMSO-d₆, 500 MHz)+TFA: δ (ppm) 9.40-9.65 (m, 2H), 8.96 (br d, J=9.8 Hz, 1H), 7.65 (d, J=8.8 Hz, 2H), 7.43 (d, J=2.0 Hz, 1H), 7.36 (d, J=8.3 Hz, 1H), 7.31 (d, J=8.3 Hz, 2H), 7.21 (dd, J=8.3, 2.0 Hz, 1H), 4.89 (br dd, J=9.9, 4.0 Hz, 1H), 3.20-3.66 (m, 10H).

### Example 115: N-(1-(3,4-dichlorophenyl)-2-(dimethylamino)ethyl)-4-(2-(trifluoromethyl)phenoxy)benzenesulfonamide (238)

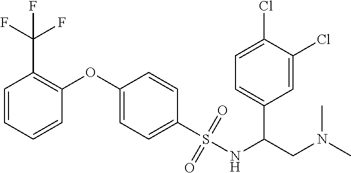 [SMILES: CN(C)CC(NS(=O)(=O)c1ccc(Oc2ccccc2C(F)(F)F)cc1)c1ccc(Cl)c(Cl)c1]

In a three necked round bottomed flask equipped with a thermometer, a solution of 4-[2-(trifluoromethyl)phenoxy]aniline (1.25 g, 4.94 mmol) in TFA (16.783 mL) was stirred at 0° C. copper(I) chloride (15 mg, 0.148 mmol) was added, the mixture was stirred at 0° C. for 5 min then a solution of sodium nitrite (432 mg, 6.17 mmol) in water (1.8648 mL) was added dropwise over 5 min to keep the temperature below 0° C. The mixture was stirred at −5° C. for 30 min to afford Solution A. In a three necked round bottomed flask equipped with a thermometer, a solution of copper(II) dichloride (338 mg, 2.52 mmol) and concentrated HCl (1.6 mL, 54.3 mmol) in AcOH (16.783 mL) and H₂SO₄ (17 mL, 0.217 mol) was stirred at −5° C. Solution A was added dropwise and the mixture was stirred at 0° C. for 5 min, then at 60° C. for 5.5 h. The mixture was extracted twice with DCM. The combined organic layers were washed with brine, dried over sodium sulfate, filtered and concentrated. Crude was diluted in DCM (1 mL). In a sealed tube under nitrogen, the obtained suspension was added to a stirred solution of 1-(3,4-dichlorophenyl)-N′,N′-dimethyl-ethane-1,2-diamine (138 mg, 0.594 mmol) and pyridine (192 μL, 2.38 mmol) in DCM (2 mL). The mixture was stirred at rt for 16 h then poured into a half-saturated aqueous NaHCO₃. The aqueous layer was extracted with DCM and the combined organic layers were washed with brine, dried over sodium sulfate, filtered and concentrated. The crude was purified by flash chromatography on silica gel using a gradient of (MeOH+2% NH₄OH) in DCM from 0.2% to 5%. The desired fractions were concentrated and purified by reverse phase chromatography on silica gel using a gradient of (MeCN+0.2% AcOH) in (H₂O+0.2% AcOH) from 0% to 100%. The desired fractions were concentrated and the residue was dissolved in Et₂O. The solution was added to stirred HCl 2 N in Et₂O and the mixture was stirred at rt for 2 h. The suspension was filtered, the residue was washed with Et₂O and dried under reduced pressure at 45° C. for 16 h to afford the title compound as a white powder (11 mg, 99.8% purity, 3% yield, tᵣ=1.79 min). LCMS (Method C): m/z found 533.2 [M+H]⁺; ¹H-NMR (DMSO-d₆, 500 MHz) δ (ppm) 9.72-9.26 (m, 1H), 8.91-8.61 (m, 1H), 7.84 (dd, J=7.8, 1.1 Hz, 1H), 7.80-7.72 (m, 1H), 7.54 (d, J=9.2 Hz, 2H), 7.53-7.49 (m, 1H), 7.48-7.41 (m, J=8.4 Hz, 2H), 7.28-7.17 (m, 1H), 6.98 (d, J=8.2 Hz, 1H), 6.82 (d, J=8.8 Hz, 2H), 4.89-4.77 (m, 1H), 3.36-3.32 (m, 1H), 3.26-3.15 (m, 1H), 3.01-2.79 (m, 6H).

### Example 116: N-(1-(3,4-dichlorophenyl)-2-(dimethylamino)ethyl)-4-(tetrahydro-2H-pyran-2-yl)benzenesulfonamide (293)

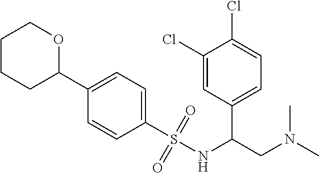 [SMILES: CN(C)CC(NS(=O)(=O)c1ccc(C2CCCCO2)cc1)c1ccc(Cl)c(Cl)c1]

### Step 1: Synthesis of 1-(4-bromophenyl)-5-chloro-pentan-1-ol

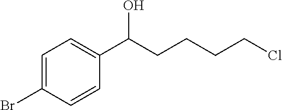 [SMILES: OC(CCCCCl)c1ccc(Br)cc1]

In a round-bottomed flask, to a stirred solution of 1-(4-bromophenyl)-5-chloropentan-1-one (97%, 1.00 g, 3.52 mmol) in THF (25 mL) and water (2.5 mL) at room temperature was added sodium borohydride (200 mg, 5.28 mmol). The reaction mixture was stirred at room temperature for 18 h. The reaction mixture was quenched with MeOH (5 mL) and concentrated under reduced pressure. The residue was partitioned between ethyl acetate (50 mL) and water (50 mL). The aqueous layer was extracted with ethyl acetate (1×25 mL). The combined organic layers were washed with a saturated aqueous solution of sodium chloride, dried over sodium sulfate, filtered and concentrated under reduced pressure. to afford the title compound as a colorless oil (1.068 g, 87% purity, 95% yield, tᵣ=0.91 min). LCMS (Method F); ¹H-NMR (DMSO-d₆, 400 MHz): δ (ppm) 7.56-7.40 (m, 2H), 7.35-7.21 (m, 2H), 5.24 (d, J=4.5 Hz, 1H), 4.58-4.44 (m, 1H), 3.61 (t, J=6.6 Hz, 2H), 1.77-1.64 (m, 2H), 1.65-1.51 (m, 2H), 1.50-1.28 (m, 2H).

### Step 2: Synthesis of 2-(4-bromophenyl)tetrahydropyran

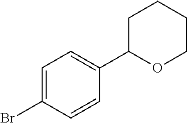 [SMILES: Brc1ccc(C2CCCCO2)cc1]

In a sealed vial, to a stirred solution of 1-(4-bromophenyl)-5-chloro-pentan-1-ol (500 mg, 1.80 mmol) in anhydrous THF (12 mL) at 0° C. under nitrogen was added potassium tert-butoxide (303 mg, 2.70 mmol). The reaction mixture was stirred at room temperature for 36 h. The reaction mixture was poured in a saturated aqueous solution of ammonium chloride (15 mL) and water (50 mL) and ethyl acetate (50 mL) were added. The aqueous layer was extracted with ethyl acetate (1×30 mL). The combined organic layers were washed with a saturated aqueous solution of sodium chloride, dried over sodium sulfate, filtered and concentrated under reduced pressure. The crude was purified by flash chromatography on silica gel using a gradient of ethyl acetate in cyclohexane from 00% to 10% to afford the title compound as a white powder (413 mg, 100% purity, 95% yield, tᵣ=1.01 min) LCMS (Method F); ¹H-NMR (DMSO-d₆, 400 MHz): δ (ppm) 7.59-7.43 (m, 2H), 7.35-7.22 (m, 2H), 4.30 (dd, J=11.2, 2.1 Hz, 1H), 4.10-3.92 (m, 1H), 3.59-3.46 (m, 1H), 1.92-1.74 (m, 2H), 1.73-1.49 (m, 3H), 1.44-1.30 (in, 1H).

### Step 3: Synthesis of 2-(4-benzylsulfanylphenyl)tetrahydropyran

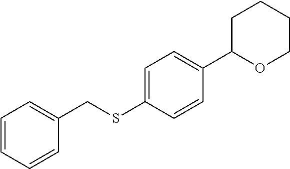 [SMILES: c1ccc(CSc2ccc(C3CCCCO3)cc2)cc1]

A sealed vial was charged with 2-(4-bromophenyl)tetrahydropyran (100%, 413 mg, 1.71 mmol) and N-ethyl-N-isopropyl-propan-2-amine (0.60 mL, 3.43 mmol) in 1,4-Dioxane-Anhydrous (8.5642 mL). The reaction mixture was degassed with argon for 5 min. Then (5-diphenylphosphanyl-9,9-dimethyl-xanthen-4-yl)-diphenyl-phosphane (98%, 101 mg, 0.171 mmol), Pd₂dba₃ (95%, 83 mg, 0.0856 mmol) and benzylmercaptan (0.40 mL, 3.43 mmol) were added and the reaction mixture was stirred at 110° C. overnight. The mixture was cooled to rt, filtered on a pad of dicalite, concentrated in vacuo and purified by flash chromatography on silica gel using a gradient of EtOAc in Heptane from 0 to 10% to afford the title compound as a white solid (403 mg, 100% purity, 83% yield, tᵣ=1.09 min) LCMS (Method F): m/z found 285.2 [M+H]*; ¹H-NMR (DMSO-d₆, 400 MHz): δ (ppm) 7.56-7.02 (m, 9H), 4.26 (dd, J=11.1, 2.1 Hz, 1H), 4.05-3.95 (m, 1H), 3.55-3.45 (m, 1H), 1.89-1.80 (m, 1H), 1.75 (dt, J=13.0, 2.6 Hz, 1H), 1.62 (dtt, J=15.5, 8.7, 3.0 Hz, 1H), 1.53 (ddq, J=10.1, 7.0, 3.5 Hz, 2H), 1.45-1.31 (in, 1H).

### Step 4: Synthesis of N-[1-(3,4-dichlorophenyl)-2-(dimethylamino)ethyl]-4-tetrahydropyran-2-yl-benzenesulfonamide

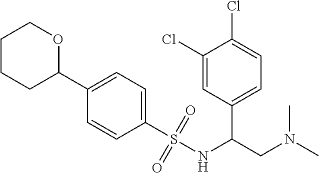 [SMILES: CN(C)CC(NS(=O)(=O)c1ccc(C2CCCCO2)cc1)c1ccc(Cl)c(Cl)c1]

In a round-bottomed flask, to a stirred solution of 2-(4-benzylsulfanylphenyl)tetrahydropyran (100 mg, 0.352 mmol) in acetonitrile (2.9457 mL) at 0° C. under nitrogen were added successively water (0.0748 mL), AcOH (0.1131 mL) and then 1,3-dichloro-5,5-dimethyl-imidazolidine-2,4-dione (139 mg, 0.703 mmol). The reaction mixture was stirred at 0° C. for 2 h, then concentrated in vacuo, dissolved in DCM and quenched with 10 mL NaHCO₃5% at 0° C. the organic layer was dried through phase separator and added dropwise to a solution of 1-(3,4-dichlorophenyl)-N²,N²-dimethylethane-1,2-diamine (95%, 81 mg, 0.331 mmol) and triethylamine (0.034 mL, 0.243 mmol) in DCM, and stirred over the weekend at rt. A half saturated solution of NaHCO₃ was added, and the mixture was extracted twice with DCM. The combined organic layers were dried over phase separator, concentrated in vacuo and purified by flash chromatography on silica gel using a gradient of MeOH (0.7N NH₃) in DCM from 1% to 5%. Desired fractions were concentrated in vacuo, dissolved in diethyl ether, and 2 M hydrogen chloride in diethyl ether (0.18 mL, 0.352 mmol) was added. The mixture was stirred overnight at rt The solid was filtered, washed with diethyl ether, and dried overnight at 50° C. under vacuum to afford the title compound as a white powder (82 mg, 98.98% purity, 47% yield, tᵣ=1.67 min). LCMS (Method C): m/z found 457 [M+H]⁺; ¹H-NMR (600 MHz, DMSO-d₆, 298K) 6 ppm 9.64 (br s, 1H), 8.69 (d, J=9.1 Hz, 1H), 7.47 (d, J=8.4 Hz, 2H), 7.43 (t, J=2.1 Hz, 1H), 7.32 (d, J=8.4 Hz, 1H), 7.21 (dd, J=8.4, 1.6 Hz, 2H), 7.17 (dd, J=8.4, 2.1 Hz, 1H), 4.80 (br s, 1H), 4.27 (dt, J=11.2, 2.6 Hz, 1H), 3.90-4.12 (m, 1H), 3.49 (td, J=11.2, 3.6 Hz, 1H), 3.10-3.30 (m, 2H), 2.73-2.97 (m, 6H), 1.78-1.90 (m, 1H), 1.58-1.75 (m, 2H), 1.45-1.57 (m, 2H), 1.09-1.32 (m, 1H).

### Example 117: 3-cyano-N-(1-(3,4-dichlorophenyl)-2-(dimethylamino)ethyl)-4-(trifluoromethoxy)benzenesulfonamide (240)

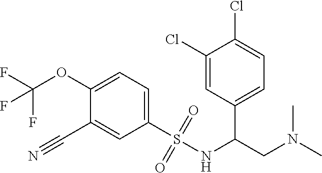 [SMILES: CN(C)CC(NS(=O)(=O)c1ccc(OC(F)(F)F)c(C#N)c1)c1ccc(Cl)c(Cl)c1]

Step 1: Synthesis of 5-benzylsulfanyl-2-(trifluoromethoxy)benzonitrile

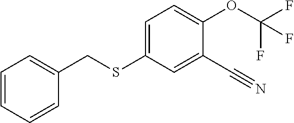 [SMILES: N#Cc1cc(SCc2ccccc2)ccc1OC(F)(F)F]

A sealed vial was charged with benzylmercaptan (240 μL, 2.04 mmol), 5-bromo-2-(trifluoromethoxy)benzonitrile (96%, 283 mg, 1.02 mmol) and Xantphos (59 mg, 0.102 mmol) in anhydrous 1,4-dioxane (10 mL). The reaction mixture was degassed with argon for 5 min. Then, tris(dibenzylideneacetone)dipalladium(0) (Pd₂dba₃) (47 mg, 0.0511 mmol) and DIPEA (357 μL, 2.04 mmol) were added and The reaction mixture was stirred at 105° C. for 21 h. The mixture was cooled to rt, and an aqueous saturated solution of Na₂S₂O₃ was added. The mixture was filtered through a pad of dicalite and the organic layer was concentrated under vacuum pressure. The crude was purified by flash chromatography on silica gel using a gradient of EtOAc in heptane from 5% to 50%. The desired fractions were combined and concentrated to afford the title compound as an orange solid (303.7 mg, 77.871% yield, tᵣ=1.03 min). LCMS (Method F); ¹H-NMR (400 MHz, DMSO-d₆) δ (ppm) 8.03 (d, J=2.4 Hz, 1H), 7.77 (dd, J=8.8, 2.4 Hz, 1H), 7.60 (dd, J=8.8, 1.4 Hz, 1H), 7.42-7.37 (m, 2H), 7.36-7.30 (m, 2H), 7.30-7.25 (m, 1H), 4.39 (s, 2H).

### Step 2: Synthesis of 3-cyano-N-[1-(3,4-dichlorophenyl)-2-(dimethylamino)ethyl]-4-(trifluoromethoxy)benzenesulfonamide

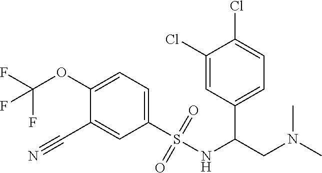 [SMILES: CN(C)CC(NS(=O)(=O)c1ccc(OC(F)(F)F)c(C#N)c1)c1ccc(Cl)c(Cl)c1]

In a round-bottomed flask, to a stirred solution of 5-benzylsulfanyl-2-(trifluoromethoxy)benzonitrile (81%, 292 mg, 0.765 mmol) in acetonitrile (7.2101 mL) at 0° C. under air were added successively water (179.84 μL), AcOH (269.9 μL) and then 1,3-dichloro-5,5-dimethyl-imidazolidine-2,4-dione (98%, 308 mg, 1.53 mmol). The reaction mixture was stirred at 0° C. for 2 h, then triethylamine (0.12 mL, 0.842 mmol) and 1-(3,4-dichlorophenyl)-N′,N′-dimethyl-ethane-1,2-diamine (98%, 273 mg, 1.15 mmol) were added in sequence (the base was added dropwise), and the mixture was warmed to rt, and stirred overnight. The mixture was concentrated in vacuo. Water and DCM were added to the residue and the aqueous layer was extracted with DCM. The combined organic layers were washed with a saturated aqueous solution of NaCl, dried using a phase separator, concentrated under reduced pressure and purified by flash chromatography on silica gel using a gradient of Methanol in DCM from 0.5% to 10%. Desired fractions were gathered, concentrated and the resulting solid was purified by flash chromatography on silica gel using a gradient of EtOH in DCM from 0.5% to 5%. Desired fractions were gathered, concentrated in vacuo, and the resulting solid was purified by reverse-phase preparative chromatography using a gradient of acetonitrile in water from 0% to 100% (0.10% AcOH in water). The desired fractions were combined and concentrated. The resulting solid was purified by flash chromatography on silica gel using an isocratic gradient (4% MeOH in DCM). The desired fractions were combined and concentrated. The resulting residue was solubilized in Et₂O and the homogenous solution was added to 2 M hydrogen chloride (4.0 mL, 8.00 mmol) in Et₂O. The resulting suspension was stirred at rt overnight. The precipitate formed was filtered, washed with Et₂O and dried under vacuum at 45° C. overnight to afford the title compound as a white powder (20.7 mg, 97.2% purity, 5% yield, tᵣ=1.41 min). LCMS (Method C): m/z found 482 [M+H]⁺; ¹H-NMR (DMSO-d₆, 600 MHz) δ (ppm) 9.47-9.78 (m, 1H), 8.86-9.25 (m, 1H), 8.09-8.15 (m, 1H), 7.91-7.96 (m, 1H), 7.61-7.68 (m, 1H), 7.46-7.52 (m, 1H), 7.39-7.43 (m, 1H), 7.20-7.31 (m, 1H), 4.80-5.03 (m, 1H), 3.38-3.53 (m, 1H), 3.16-3.26 (m, 1H), 2.72-3.03 (m, 6H).

### Example 118: N-(1-(3,4-dichlorophenyl)-2-(4-propoxypiperidin-1-yl)ethyl)-4-(trifluoromethoxy)benzenesulfonamide (241)

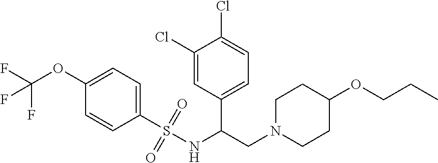 [SMILES: CCCOC1CCN(CC(NS(=O)(=O)c2ccc(OC(F)(F)F)cc2)c2ccc(Cl)c(Cl)c2)CC1]

### Step 1: 1-(3,4-dichlorophenyl)-2-(4-propoxy-1-piperidyl)ethenone

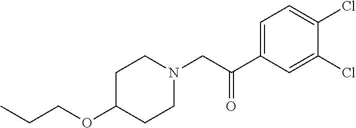 [SMILES: CCCOC1CCN(CC(=O)c2ccc(Cl)c(Cl)c2)CC1]

In a sealed tube under nitrogen, a solution of 2-bromo-1-(3,4-dichlorophenyl)ethanone (97%, 500 mg, 1.81 mmol) and 4-propoxypiperidine hydrochloride (1:1) (95%, 514 mg, 2.72 mmol) in anhydrous DCM (10 mL) was stirred at rt. triethylamine (656 μL, 4.71 mmol) then N,N-dimethylpyridin-4-amine (11 mg, 0.0905 mmol) were added and the solution was stirred at rt for 18 h. The mixture was washed with a half saturated aqueous solution of NaHCO₃. The aqueous layer was extracted twice with DCM. The combined organic layers were washed with brine, dried using a phase separator and concentrated under reduce pressure. The crude was purified by flash chromatography on silica gel using a gradient of MeOH in DCM from 0.5% to 5% to afford the title compound as a yellow oil. (297 mg, 37% yield, tᵣ=0.63 min). LCMS (Method F): m/z found 330.2 [M+H]⁺; ¹H-NMR (DMSO-d₆, 400 MHz) δ (ppm) 8.19 (d, J=2.0 Hz, 1H), 7.95 (dd, J=8.4, 2.0 Hz, 1H), 7.80 (d, J=8.4 Hz, 1H), 3.80 (s, 2H), 3.39-3.33 (m, 3H), 2.72 (dt, J=10.1, 4.3 Hz, 2H), 2.24 (td, 2H), 1.79 (dd, J=13.2, 3.8 Hz, 2H), 1.54-1.37 (m, 4H), 0.89-0.84 (m, 3H).

### Step 2: Synthesis of 1-(3,4-dichlorophenyl)-2-(4-propoxy-1-piperidyl)ethanone oxime

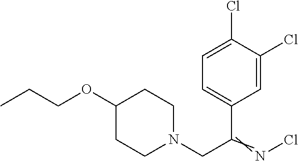 [SMILES: CCCOC1CCN(CC(=NCl)c2ccc(Cl)c(Cl)c2)CC1]

A round bottom flask was charged with 1-(3,4-dichlorophenyl)-2-(4-propoxy-1-piperidyl)ethanone (297 mg, 0.898 mmol), hydroxylamine hydrochloride (125 mg, 1.80 mmol) and DIPEA (0.33 mL, 1.89 mmol) in anhydrous EtOH (10 mL). The flask was sealed and the reaction mixture was stirred at 80° C. overnight. The reaction mixture was concentrated under reduced pressure and dried under vacuum to afford the title compound as an orange solid (603 mg, quant., tᵣ=0.65 min). LCMS (Method F): m/z found 345.3 [M+H]⁺.

### Step 3: Synthesis of 1-(3,4-dichlorophenyl)-2-(4-propoxy-1-piperidyl)ethanamine

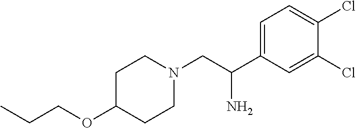 [SMILES: CCCOC1CCN(CC(N)c2ccc(Cl)c(Cl)c2)CC1]

In a round bottom flask under atmospheric pressure, to a stirred solution of 1-(3,4-dichlorophenyl)-2-(4-propoxy-1-piperidyl)ethanone oxime (310 mg, 0.898 mmol) in anhydrous THF (10 mL) at 0° C. under nitrogen was added 2.4 M LAH in THF (1.1 mL, 2.69 mmol). The reaction mixture was stirred at 70° C. for 3 h. The reaction mixture was quenched at 0° C. with water (0.102 mL, same weight as LAH used), 1 M NaOH (0.102 mL) was added and water (three times the weight of LAH used). The resulting heterogenous mixture was stirred at room temperature for 15 min, filtered through a pad of dicalite, dried over sodium sulfate, rinsed with THF, and concentrated under reduced pressure. The crude was purified by flash chromatography on silica gel using a gradient of (0.7N NH₃) methanol in dichloromethane from 2% to 8% to afford the title compound as a yellow oil (94.2 mg, 31% yield, tᵣ=0.53 min). LCMS (Method F): m/z found 331.3 [M+H]⁺; ¹H-NMR (400 MHz, DMSO-d₆) δ (ppm): 7.64 (d, J=2.0 Hz, 1H), 7.54 (d, J=8.3 Hz, 1H), 7.36 (dd, J=8.3, 2.0 Hz, 1H), 4.05-3.99 (m, 1H), 3.33 (t, J=6.5 Hz, 2H), 3.28-3.19 (m, 1H), 2.81-2.60 (m, 2H), 2.31-2.15 (m, 3H), 2.09-2.00 (m, 1H), 1.99 (s, 2H), 1.78 (s, 2H), 1.47 (m, J=7.3 Hz, 2H), 1.44-1.33 (m, 2H), 0.86 (d, J=7.3 Hz, 3H).

### Step 4: Synthesis of N-(1-(3,4-dichlorophenyl)-2-(4-propoxypiperidin-1-yl)ethyl)-4-(trifluoromethoxy)benzenesulfonamide

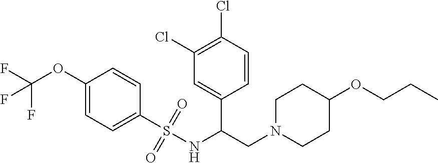 [SMILES: CCCOC1CCN(CC(NS(=O)(=O)c2ccc(OC(F)(F)F)cc2)c2ccc(Cl)c(Cl)c2)CC1]

In a sealed tube, 4-(trifluoromethoxy)benzenesulfonyl chloride (97%, 55 μL, 0.313 mmol) was added to a stirred solution of triethylamine (119 μL, 0.853 mmol) and 1-(3,4-dichlorophenyl)-2-(4-propoxy-1-piperidyl)ethanamine (94 mg, 0.284 mmol) in DCM (2.5 mL). The solution was stirred at room temperature for 18 h. Water (5 mL) was added and the aqueous layer was extracted with dichloromethane (2×10 mL). The combined organic layers were washed with a saturated aqueous solution of sodium chloride, dried over a phase separator and concentrated under reduced pressure. The crude was purified by flash chromatography on silica gel using a gradient of MeOH (0.7M NH₃) in DCM from 0.5% to 2%. The desired fractions were combined and concentrated. The resulting residue was solubilized in Et₂O and the homogenous solution was added to 2 M hydrogen chloride (1.4 mL, 2.84 mmol). The resulting suspension was stirred overnight at rt. The precipitate formed was filtered, washed with Et₂O and dried under vacuum at 50° C. for 24 h to afford the title compound as a white powder (125.2 mg, 100% purity, 74% yield, tᵣ=1.86 min). LCMS (Method C): m/z found 555 [M+H]⁺; ¹H-NMR (DMSO-d₆, 500 MHz) δ (ppm) 9.7-10.1 (m, 1H), 8.8-8.9 (m, 1H), 7.67 (d, 2H, J=8.8 Hz), 7.45 (s, 1H), 7.37 (br d, 1H, J=8.3 Hz), 7.32 (br d, 2H, J=8.3 Hz), 7.22 (br d, 1H, J=8.3 Hz), 4.92 (dt, 1H, J=3.4, 9.7 Hz), 2.8-4.1 (m, 9H), 1.6-2.2 (m, 4H), 1.4-1.6 (m, 2H), 0.7-1.0 (m, 3H).

### Example 119: N-(1-(3,4-dichlorophenyl)-2-(methylamino)ethyl)-4-(trifluoromethoxy)benzenesulfonamide (242)

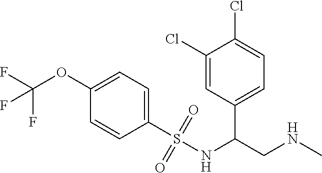 [SMILES: CNCC(NS(=O)(=O)c1ccc(OC(F)(F)F)cc1)c1ccc(Cl)c(Cl)c1]

### Step 1: Synthesis of methyl (2R)-2-(3,4-dichlorophenyl)-2-[[4-(trifluoromethoxy)phenyl]sulfonylamino]acetate

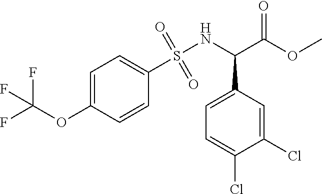 [SMILES: COC(=O)[C@H](NS(=O)(=O)c1ccc(OC(F)(F)F)cc1)c1ccc(Cl)c(Cl)c1]

In a sealed tube under nitrogen, A suspension of 4-(trifluoromethoxy)benzenesulfonyl chloride (992 mg, 3.81 mmol) in DCM (15 mL) was stirred at 0° C. Methyl (2R)-2-amino-2-(3,4-dichlorophenyl)acetate hydrochloride (1.03 g, 3.81 mmol) was added and the mixture was stirred 5 min at 0° C. A solution of triethylamine (2.1 mL, 15.2 mmol) in DCM (3 mL) was added and the mixture was stirred at rt for 64 h. The mixture was diluted with DCM and half saturated aqueous NaHCO₃. The aqueous layer was extracted with DCM. The combined organic layers were washed with brine, dried over sodium sulfate, filtered and concentrated. The crude was purified by flash chromatography on silica gel using a gradient of (MeOH+2% NH₄OH) in DCM from 0.2% to 2% to afford the title compound as a yellow solid (550 mg, 96% purity, 30% yield, tᵣ=0.98 min). LCMS (Method F); ¹H-NMR (DMSO-d₆, 400 MHz): δ (ppm) 9.13 (s, 1H), 7.83-7.70 (m, 2H), 7.54-7.36 (m, 4H), 7.27 (dd, J=8.4, 2.1 Hz, 1H), 5.27 (s, 1H), 3.54 (s, 3H).

### Step 2: Synthesis of 2-(3,4-dichlorophenyl)-N-methyl-2-[[4-(trifluoromethoxy)phenyl]sulfonylamino]acetamide

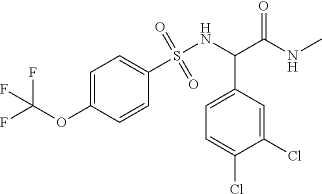 [SMILES: CNC(=O)C(NS(=O)(=O)c1ccc(OC(F)(F)F)cc1)c1ccc(Cl)c(Cl)c1]

In a sealed tube under nitrogen, 2 M methylamine in THF (262 μL, 0.524 mmol) was added to a stirred solution of methyl (2R)-2-(3,4-dichlorophenyl)-2-[[4-(trifluoromethoxy)phenyl]sulfonylamino]acetate (96%, 100 mg, 0.209 mmol) and dichloro(dicyclopenta-2,4-dien-1-yl)zirconium (9.2 mg, 0.0314 mmol) in dry toluene (1 mL). The mixture was stirred at 110° C. for 16 h and was cooled to rt. Additional 2 M methylamine in THF (0.26 mL, 0.524 mmol) and dichloro(dicyclopenta-2,4-dien-1-yl)zirconium (9.2 mg, 0.0314 mmol) were added and the mixture was stirred at 110° C. for 3 h. The mixture was concentrated and the crude was purified by flash chromatography on silica gel using a gradient of (MeOH+2% NH₄OH) in DCM from 1% to 10% to afford the title compound as a beige solid (30 mg, 88% purity, 27% yield, tᵣ=0.88 min). LCMS (Method F): m/z found 457.1 [M+H]⁺; ¹H-NMR (DMSO-d₆, 400 MHz): δ (ppm) 8.84 (d, J=6.0 Hz, 1H), 8.23 (d, J=4.7 Hz, 1H), 7.79-7.69 (m, 2H), 7.48-7.34 (m, 4H), 7.21 (dd, J=8.4, 2.1 Hz, 1H), 5.00 (d, J=6.4 Hz, 1H), 2.46 (d, J=4.6 Hz, 3H).

### Step 3: Synthesis of N-[1-(3,4-dichlorophenyl)-2-(methylamino)ethyl]-4-(trifluoromethoxy)benzenesulfonamide

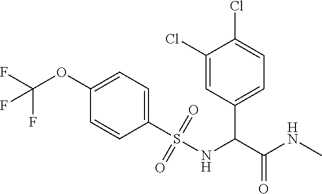 [SMILES: CNC(=O)C(NS(=O)(=O)c1ccc(OC(F)(F)F)cc1)c1ccc(Cl)c(Cl)c1]

In a sealed tube under nitrogen, a solution of 2 M borane-methyl sulfide complex (87 μL, 0.173 mmol) in dry THF (1 mL) was stirred at 0° C. 2-(3,4-dichlorophenyl)-N-methyl-2-[[4-(trifluoromethoxy)phenyl]sulfonylamino]acetamide (27 mg, 0.0590 mmol) was added dropwise and the mixture was stirred at rt for 15 min and at 60° C. for 16 h then was cooled to 0° C. MeOH (100 μL) was added dropwise at 0° C. and the mixture was stirred at 0° C. for 15 min. 4 N aqueous HCl (100 μL) was added and the mixture was stirred at 60° C. for 1 h. The mixture was cooled to 0° C., quenched with aqueous saturated NaHCO₃ then extracted twice with EtOAc. The combined organic layers were washed with brine, dried over sodium sulfate, filtered and concentrated. The crude was purified by flash chromatography on silica gel using a gradient of (MeOH+2% NH₄OH) in DCM from 1% to 10%. The desired fractions were concentrated and the residue was dissolved in Et₂O. The solution was added to stirred 2N HCl/Et₂O and the mixture was stirred at rt for 3 h. The suspension was filtered, the residue was washed with Et₂O and dried under reduced pressure at 50° C. for 64 h to afford the title compound as a white powder (9 mg, 100% purity, 32% yield, tᵣ=1.66 min). LCMS (Method C): m/z found 443 [M+H]⁺; ¹H-NMR (DMSO-d₆, 600 MHz): δ (ppm) 8.67-9.03 (m, 2H), 8.44-8.66 (m, 1H), 7.62 (d, J=8.7 Hz, 2H), 7.37 (d, J=8.4 Hz, 1H), 7.33 (br d, J=2.5 Hz, 1H), 7.31 (d, J=8.5 Hz, 2H), 7.09-7.13 (m, 1H), 4.68 (br d, J=5.3 Hz, 1H), 3.08-3.23 (m, 2H), 2.59 (s, 3H).

### Example 120: N-((1-(3,4-dichlorophenyl)cyclopentyl)methyl)-4-(trifluoromethoxy)benzenesulfonamide (243)

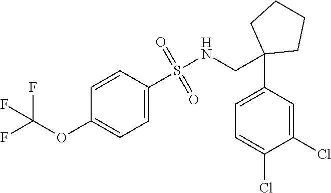 [SMILES: O=S(=O)(NCC1(c2ccc(Cl)c(Cl)c2)CCCC1)c1ccc(OC(F)(F)F)cc1]

### Step 1: Synthesis of 1-(3,4-dichlorophenyl)cyclopentanecarbonitrile

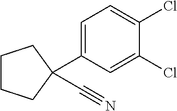 [SMILES: N#CC1(c2ccc(Cl)c(Cl)c2)CCCC1]

In a three necked round bottomed flask equipped with a thermometer, under nitrogen, a suspension of sodium hydride (60%, 591 mg, 14.8 mmol) in dry DMF (16 mL) was stirred at 0° C. A solution of (3,4-dichlorophenyl)acetonitrile (1.00 g, 5.38 mmol) in dry DMF (6 mL) was added dropwise and the mixture was stirred at 0° C. for 30 min. A solution of 1,4-dibromobutane (1.74 g, 8.06 mmol) in dry DMF (6 mL) was added dropwise and the mixture was stirred at rt for 3 h. The mixture was stirred at 0° C. and half saturated aqueous NH₄Cl (70 mL) was added dropwise. EtOAc was added and the mixture was stirred at rt for 10 min. The aqueous layer was extracted with EtOAc. The combined organic layers were washed with brine, dried over sodium sulfate, filtered and concentrated. The crude was purified by flash chromatography on silica gel using a gradient of EtOAc in heptane from 1% to 5% to afford the title compound as a colorless liquid (1.12 g, 86% yield, tᵣ=1.01 min). LCMS (Method F): m/z found 294.0 [M+H]⁺; ¹H-NMR (DMSO-d₆, 400 MHz): δ (ppm) 7.77 (d, J=2.3 Hz, 1H), 7.70 (d, J=8.5 Hz, 1H), 7.53 (dd, J=8.5, 2.3 Hz, 1H), 2.49-2.39 (m, 2H), 2.14-2.02 (m, 2H), 1.95-1.85 (m, 3H).

### Step 2: Synthesis of [1-(3,4-dichlorophenyl)cyclopentyl]methanamine

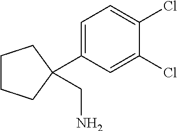 [SMILES: NCC1(c2ccc(Cl)c(Cl)c2)CCCC1]

In a three necked round bottomed flask equipped with a thermometer, under nitrogen, a solution of 1-(3,4-dichlorophenyl)cyclopentanecarbonitrile (1.12 g, 4.66 mmol) in dry THF (20 mL) was stirred at 0° C. 2.4 M lithium aluminum hydride (4.9 mL, 11.7 mmol) was added dropwise and the mixture was stirred at rt for 2 h. The mixture was vigorously stirred at 0° C. and water (500 μL) was added dropwise. The mixture was stirred 10 min at 0° C. then aqueous NaOH 15% (500 μL) was added dropwise. The mixture was stirred at 0° C. for 10 min then water (1.5 mL) was added dropwise. The mixture was stirred at 0° C. for 30 min, then EtOAc and Na₂SO₄ were added, and the mixture was stirred at rt for 1 h. The mixture was filtered, the residue was washed with EtOAc and the filtrate was concentrated. The crude was purified by flash chromatography on silica gel using a gradient of (MeOH+2% NH₄OH) in DCM from 1% to 10% to afford the title compound as a colorless oil (750 mg, 63% yield, tᵣ=0.62 min). LCMS (Method F): m/z found 244.2 [M+H]⁺; ¹H-NMR (DMSO-d₆, 400 MHz): δ (ppm) 7.53 (d, J=8.4 Hz, 1H), 7.45 (d, J=2.2 Hz, 1H), 7.25 (dd, J=8.4, 2.2 Hz, 1H), 2.59 (s, 2H), 1.99-1.89 (m, 2H), 1.75-1.54 (m, 6H), 1.06 (s, 2H).

### Step 3: N-((1-(3,4-dichlorophenyl)cyclopentyl)methyl)-4-(trifluoromethoxy)benzenesulfonamide

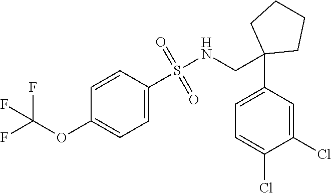 [SMILES: O=S(=O)(NCC1(c2ccc(Cl)c(Cl)c2)CCCC1)c1ccc(OC(F)(F)F)cc1]

In a sealed tube, triethylamine (228 μL, 1.64 mmol) was added to a stirred solution of 4-(trifluoromethoxy)benzenesulfonyl chloride (107 mg, 0.410 mmol) and [1-(3,4-dichlorophenyl)cyclopentyl]methanamine (100 mg, 0.410 mmol) in DCM (3 mL). The solution was stirred at rt for 16 h and was diluted with half saturated aqueous NaHCO₃. The aqueous layer was extracted with DCM, the combined organic layers were washed with brine, dried over sodium sulfate, filtered and concentrated. The crude was purified by flash chromatography on silica gel using a gradient of EtOAc in heptane from 2% to 20%. The desired fractions were concentrated and the residue was triturated in pentane+a few drops Et₂O. The obtained suspension was filtered, the residue was washed with pentane and dried under reduced pressure at 45° C. for 16 h to afford the title compound as a white powder (115 mg, 98.6% purity, 59% yield, tᵣ=3.17 min). LCMS (Method C): m/z found 468.2 [M+H]⁺; ¹H-NMR (DMSO-d₆, 400 MHz) δ (ppm) 7.80 (d, J=8.0 Hz, 2H), 7.47-7.56 (m, 4H), 7.42 (d, J=2.1 Hz, 1H), 7.22 (dd, J=8.5, 2.3 Hz, 1H), 2.90 (s, 2H), 1.92-2.02 (m, 2H), 1.53-1.76 (m, 6H).

### Example 121: N-((1-(4-chlorophenyl)cyclopentyl)methyl)-4-(trifluoromethoxy)benzenesulfonamide (244)

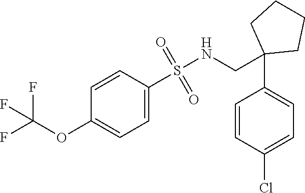 [SMILES: O=S(=O)(NCC1(c2ccc(Cl)cc2)CCCC1)c1ccc(OC(F)(F)F)cc1]

### Step 1: Synthesis of 1-(4-chlorophenyl)cyclopentanecarbonitrile

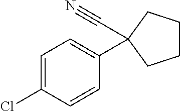 [SMILES: N#CC1(c2ccc(Cl)cc2)CCCC1]

In a three necked round bottomed flask equipped with a thermometer, a suspension of sodium hydride (60%, 726 mg, 18.1 mmol) in dry DMF (16 mL) was stirred at 0° C. A solution of (4-chlorophenyl)acetonitrile (1.00 g, 6.60 mmol) in dry DMF (6 mL) was added dropwise and the mixture was stirred at 0° C. for 30 min. A solution of 1,4-dibromobutane (2.14 g, 9.89 mmol) in dry DMF (6 mL) was added dropwise and the mixture was stirred at rt for 3 h. The mixture was stirred at 0° C. and half saturated aqueous NH₄Cl (70 mL) was added dropwise. EtOAc was added and the mixture was stirred at rt for 10 min. The aqueous layer was extracted with EtOAc. The combined organic layers were washed with brine, dried over sodium sulfate, filtered and concentrated. The crude was purified by flash chromatography on silica gel using a gradient of EtOAc in heptane from 1% to 5% to afford the title compound as a colorless liquid (1.08 g, 79% yield, tᵣ=0.95 min). LCMS (Method F); ¹H-NMR (DMSO-d₆, 400 MHz): δ (ppm) 7.57-7.51 (m, 2H), 7.51-7.46 (m, 2H), 2.46-2.36 (m, 2H), 2.13-1.99 (m, 2H), 1.96-1.82 (m, 4H).

### Step 2: [1-(4-chlorophenyl)cyclopentyl]methanamine

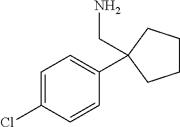 [SMILES: NCC1(c2ccc(Cl)cc2)CCCC1]

In a three necked round bottomed flask equipped with a thermometer, under nitrogen, a solution of 1-(4-chlorophenyl)cyclopentanecarbonitrile (1.08 g, 5.25 mmol) in dry THF (20 mL) was stirred at 0° C. 2.4 M lithium aluminum hydride (5.5 mL, 13.1 mmol) was added dropwise and the mixture was stirred at rt for 2 h. The mixture was vigorously stirred at 0° C. and water (500 μL) was added dropwise. The mixture was stirred 10 min at 0° C. then aqueous NaOH 15% (500 μL) was added dropwise. The mixture was stirred at 0° C. for 10 min then water (1.5 mL) was added dropwise. The mixture was stirred at 0° C. for 30 min, then EtOAc and Na₂SO₄ were added, and the mixture was stirred at rt for 1 h. The mixture was filtered, the residue was washed with EtOAc and the filtrate was concentrated. The crude was purified by flash chromatography on silica gel using a gradient of (MeOH+2% NH₄OH) in DCM from 1% to 10% to afford the title compound as a colorless oil (980 mg, 89% yield, tᵣ=0.58 min). LCMS (Method F): m/z found 210.3 [M+H]⁺; ¹H-NMR (DMSO-d₆, 400 MHz): δ (ppm) 7.38-7.30 (m, 2H), 7.30-7.24 (m, 2H), 2.57 (s, 2H), 2.02-1.86 (m, 2H), 1.78-1.49 (m, 7H), 0.98 (s, 2H)

### Step 3: Synthesis of N-((1-(4-chlorophenyl)cyclopentyl)methyl)-4-(trifluoromethoxy)benzenesulfonamide

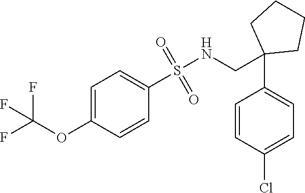 [SMILES: O=S(=O)(NCC1(c2ccc(Cl)cc2)CCCC1)c1ccc(OC(F)(F)F)cc1]

In a sealed tube, triethylamine (266 μL, 1.91 mmol) was added to a stirred solution of 4-(trifluoromethoxy)benzenesulfonyl chloride (124 mg, 0.477 mmol) and [1-(4-chlorophenyl)cyclopentyl]methanamine (100 mg, 0.477 mmol) in DCM (3 mL). The solution was stirred at rt for 16 h and was diluted with half saturated aqueous NaHCO₃. The aqueous layer was extracted with DCM, the combined organic layers were washed with brine, dried over sodium sulfate, filtered and concentrated. The crude was purified by flash chromatography on silica gel using a gradient of EtOAc in heptane from 2% to 20% to afford the title compound as a colorless oil which crystallized on standing to yield a white solid (152 mg, 99.5% purity, 73% yield, tᵣ=3.07 min). LCMS (Method C): m/z found 432.2 [M+H]⁺; ¹H-NMR (DMSO-d₆, 500 MHz) δ (ppm) 7.80 (d, 2H, J=8.8 Hz), 7.51 (br d, 3H, J=8.1 Hz), 7.3-7.3 (m, 2H), 7.2-7.3 (m, 2H), 2.87 (s, 2H), 1.94 (br dd, 2H, J=5.7, 11.4 Hz), 1.5-1.8 (m, 6H).

### Example 122: N-(2-(1-(3,4-dichlorophenyl)cyclopropyl)propan-2-yl)-4-(trifluoromethoxy)benzenesulfonamide (245)

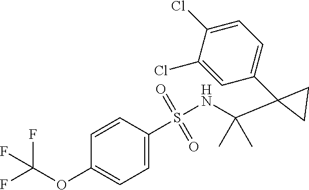 [SMILES: CC(C)(NS(=O)(=O)c1ccc(OC(F)(F)F)cc1)C1(c2ccc(Cl)c(Cl)c2)CC1]

### Step 1: Synthesis of 1-(3,4-dichlorophenyl)cyclopropanecarbonitrile

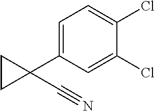 [SMILES: N#CC1(c2ccc(Cl)c(Cl)c2)CC1]

In a three-neck round-bottomed flask, to a stirred suspension of sodium hydride (60%, 579 mg, 14.5 mmol) in anhydrous DMF (13 mL) at 0° C. under nitrogen was added dropwise a solution of (3,4-dichlorophenyl)acetonitrile (98%, 1.00 g, 5.27 mmol) in anhydrous DMF (5 mL). The reaction mixture was stirred at 0° C. for 15 min (until no gas production was observed). A solution of 1,2-dibromoethane (681 μL, 7.90 mmol) in anhydrous DMF (5 mL) was added dropwise at 0° C. and then the reaction mixture was allowed to warm up to room temperature and stirred for 3 h. The reaction mixture was quenched with a saturated aqueous solution of ammonium chloride (100 mL) and ethyl acetate (75 mL) and water (50 mL) were added. The aqueous layer was extracted with ethyl acetate (1×50 mL). The combined organic layers were washed with water, a saturated aqueous solution of sodium chloride, dried over sodium sulfate, filtered and concentrated under reduced pressure. The crude was purified by flash chromatography on silica gel using a gradient of ethyl acetate in cyclohexane from 5% to 30% to afford the title compound as a yellow powder (939 mg, 84% yield, tᵣ=0.91 min). LCMS (Method F); ¹H-NMR (DMSO-d₆, 400 MHz) δ (ppm) 7.66 (d, J=8.4 Hz, 1H), 7.55 (d, J=2.3 Hz, 1H), 7.38 (dd, J=8.4, 2.3 Hz, 1H), 1.82-1.77 (m, 2H), 1.63-1.58 (m, 2H).

### Step 2: Synthesis of 2-[1-(3,4-dichlorophenyl)cyclopropyl]propan-2-amine

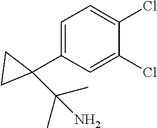

A sealed vial was charged with 1-(3,4-dichlorophenyl)cyclopropanecarbonitrile (100%, 250 mg, 1.18 mmol) and 3.4 M bromo(methyl)magnesium in MeTHF (1.0 mL, 3.54 mmol) in anhydrous THF (2 mL). The reaction mixture was irradiated at 100° C. for 10 min in a Anton Paar microwave. Ti(O-iPr)₄ (349 μL, 1.18 mmol) was added at room temperature and the reaction mixture was irradiated at 50° C. for 1 h in a Anton Paar microwave. The reaction mixture was quenched with a 20% aqueous solution of ammonium hydroxide (10 mL) and THF (50 mL) was added. The suspension was filtered, washed with THF (1×20 mL) and water (50 mL) was added to the filtrate. The aqueous layer was extracted with ethyl acetate (2×50 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5% to afford the title compound as a yellow oil (202 mg, 68% yield, tᵣ=0.60 min). LCMS (Method F): m/z found 244.2 [M+H]⁺; ¹H-NMR (DMSO-d₆, 400 MHz): δ (ppm) 7.61-7.44 (m, 2H), 7.31 (dd, J=8.3, 2.0 Hz, 1H), 1.26 (s, 2H), 1.04-0.99 (m, 2H), 0.95 (s, 6H), 0.55-0.43 (m, 2H).

### Step 3: Synthesis of N-[1-[1-(3,4-dichlorophenyl)cyclopropyl]-1-methyl-ethyl]-4-(trifluoromethoxy)benzenesulfonamide

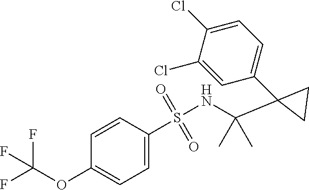 [SMILES: CC(C)(NS(=O)(=O)c1ccc(OC(F)(F)F)cc1)C1(c2ccc(Cl)c(Cl)c2)CC1]

In a sealed tube, 4-(trifluoromethoxy)benzenesulfonyl chloride (102 μL, 0.604 mmol) was added to a stirred solution of 2-[1-(3,4-dichlorophenyl)cyclopropyl]propan-2-amine (134 mg, 0.549 mmol) and triethylamine (229 μL, 1.65 mmol) in DCM (5 mL). The solution was stirred at room temperature for 3 h. was added at room temperature and the reaction mixture was stirred at 35° C. for 18 h. Water (5 mL) was added and the aqueous layer was extracted with dichloromethane (1×10 mL). The combined organic layers were washed with a saturated aqueous solution of sodium chloride, dried over sodium sulfate, filtered and concentrated under reduced pressure. The crude was purified by flash chromatography on silica gel using a gradient of ethyl acetate in cyclohexane from 5% to 30%. The residue was triturated in pentane and concentrated under reduced pressure to afford the title compound as a colorless oil (139.5 mg, 100% purity, 54% yield, tᵣ=3.33 min). LCMS (Method C): m/z found 489.8 [M+Na]⁺; ¹H-NMR (DMSO-d₆, 500 MHz) δ (ppm) 7.8-7.8 (m, 2H), 7.6-7.7 (m, 2H), 7.48 (dd, 3H, J=8.2, 15.0 Hz), 7.33 (dd, 1H, J=2.0, 8.3 Hz), 1.07 (s, 2H), 0.99 (s, 6H), 0.6-0.7 (m, 2H).

### Example 123: (R)—N-(1-(3,4-dichlorophenyl)-3-(dimethylamino)propan-2-yl)-4-(trifluoromethoxy)benzenesulfonamide (246)

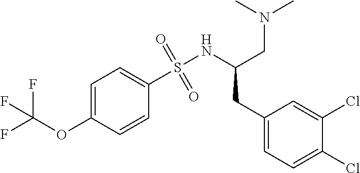 [SMILES: CN(C)C[C@@H](Cc1ccc(Cl)c(Cl)c1)NS(=O)(=O)c1ccc(OC(F)(F)F)cc1]

### Step 1: (2R)-2-(tert-butoxycarbonylamino)-3-(3,4-dichlorophenyl)propanoic acid

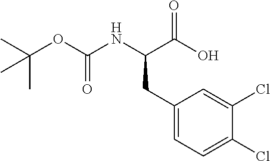 [SMILES: CC(C)(C)OC(=O)N[C@H](Cc1ccc(Cl)c(Cl)c1)C(=O)O]

In a round-bottomed flask, to a stirred solution of 3,4-dichloro-D-phenylalanine (96%, 600 mg, 2.46 mmol) in 1,4-dioxane (9.8361 mL) and water (4.918 mL) at 0° C. were added successively 1 M NaOH (4.9 mL, 4.92 mmol) and di-tert-butyl dicarbonate (591 mg, 2.71 mmol) slowly. The mixture was stirred overnight at room temperature. Ethyl acetate was added, the mixture was cooled to 0° C. and acidified to pH=3 with 1.0 M KHSO₄. Layers were separated and the aqueous layer was extracted twice more with ethyl acetate. The combined organic layers were dried over Na₂SO₄ and concentrated in vacuo to afford the title compound as a white solid (1.04 g, 100% yield, tᵣ=0.89 min). LCMS (Method F): m/z found 234.1 [M+H-Boc]⁺; ¹H-NMR (DMSO-d₆, 400 MHz): δ (ppm) 12.65 (s, 1H), 7.60-7.48 (m, 2H), 7.26 (dd, J=8.3, 1.9 Hz, 1H), 7.19-6.70 (m, 1H), 4.12 (td, J=10.5, 4.5 Hz, 1H), 3.05 (dd, J=13.8, 4.4 Hz, 1H), 2.82 (dd, J=13.7, 10.8 Hz, 1H), 1.32 (s, 9H).

### Step 2: tert-butyl N-[(1R)-1-[(3,4-dichlorophenyl)methyl]-2-(dimethylamino)-2-oxo-ethyl]carbamate

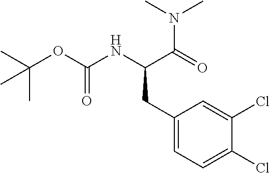

In a sealed vial under nitrogen, to a stirred solution of (2R)-2-(tert-butoxycarbonylamino)-3-(3,4-dichlorophenyl)propanoic acid (0.82 g, 2.47 mmol) and [dimethylamino(triazolo[4,5-b]pyridin-3-yloxy)methylene]-dimethyl-ammonium;hexafluorophosphate (98%, 1245 mg, 3.21 mmol) in DCM (11.217 mL) was added N-ethyl-N-isopropyl-propan-2-amine (0.65 mL, 3.70 mmol). The reaction was stirred at rt for 10 minutes then 2 M dimethylamine in THF (3.7 mL, 7.40 mmol) was added and the suspension was stirred overnight at rt. The resulting solution was diluted with DCM and an half saturated solution of NaHCO₃, and the aqueous layer was extracted twice with DCM. The organic layers were dried over phase separator, concentrated in vacuo and purified by flash chromatography on silica gel using a gradient of EtOAc in Heptane from 25% to 50% to afford the title compound as a colorless oil (661 mg, 74% yield, tᵣ=0.91 min). LCMS (Method F): m/z found 361.3 [M+H]⁺; ¹H-NMR (DMSO-d₆, 400 MHz): δ (ppm) 7.61-7.49 (m, 2H), 7.26 (dd, J=8.3, 2.0 Hz, 1H), 7.08 (d, J=8.8 Hz, 1H), 4.56 (td, J=9.3, 5.1 Hz, 1H), 2.97 (s, 3H), 2.87 (dd, J=13.7, 5.0 Hz, 1H), 2.82 (s, 3H), 2.75 (dd, J=13.5, 9.7 Hz, 1H), 1.29 (s, 9H).

### Step 3: Synthesis of tert-butyl N-[(1R)-1-[(3,4-dichlorophenyl)methyl]-2-(dimethylamino)ethyl]carbamate

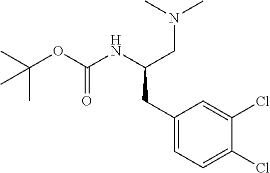

In a round bottomed flask, to a stirred solution of tert-butyl N-[(1R)-1-[(3,4-dichlorophenyl)methyl]-2-(dimethylamino)-2-oxo-ethyl]carbamate (661 mg, 1.83 mmol) in anhydrous THF (18.297 mL) at 0° C. under nitrogen was added 2.4 M LAH in THF (3.0 mL, 7.32 mmol) dropwise. The mixture was stirred at 0° C. for 1 hour and at rt for 2 hours, then the mixture was cooled to 0° C. and quenched with water (278 μL, same weight as LAH used, addition dropwise). Then 15% aqueous sodium hydroxide solution (278 μL) and water (three times the weight of LAH) were added. The resulting heterogeneous mixture was stirred at room temperature overnight, then dried over Na₂SO₄, filtered and concentrated under reduced pressure. The crude was purified by flash chromatography on silica gel using a gradient of methanol (0.7 N NH₃) in DCM from 1% to 5% to afford the title compound as a white powder (346 mg, 53% yield, tᵣ=0.66 min). LCMS (Method F): m/z found 347.3 [M+H]⁺; ¹H-NMR (DMSO-d₆, 400 MHz): δ (ppm) 7.51 (d, J=8.2 Hz, 1H), 7.42 (s, 1H), 7.17 (dd, J=8.2, 2.0 Hz, 1H), 6.65 (d, J=8.8 Hz, 1H), 3.68 (s, 1H), 2.87 (dd, J=13.7, 3.9 Hz, 1H), 2.49-2.39 (m, 1H), 2.20 (d, J=7.3 Hz, 2H), 2.16 (s, 6H), 1.29 (s, 9H).

### Step 4: (R)-3-(3,4-dichlorophenyl)-N₁,N₁-dimethylpropane-1,2-diamine

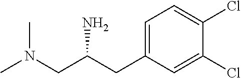

In a round-bottomed flask, to a stirred solution of tert-butyl N-[(1R)-1-[(3,4-dichlorophenyl)methyl]-2-(dimethylamino)ethyl]carbamate (346 mg, 0.996 mmol) in 1,4-dioxane (4.9816 mL) at rt was added 4 M hydrogen chloride in dioxane (5.0 mL, 19.9 mmol). The reaction mixture was stirred at rt overnight, then filtered and washed with diethyl ether, to afford the title compound as a white powder (286 mg, 81% yield, tᵣ=0.47 min). LCMS (Method E): m/z found 247.1 [M+H]⁺; ¹H-NMR (DMSO-d₆, 400 MHz): δ (ppm) 10.82 (s, 1H), 8.52 (s, 3H), 7.70 (s, 1H), 7.64 (d, J=8.2 Hz, 1H), 7.37 (d, J=8.5 Hz, 1H), 4.04 (s, 1H), 3.23 (s, 2H), 3.02 (s, 2H), 2.81 (s, 6H).

### Step 5: (R)—N-(1-(3,4-dichlorophenyl)-3-(dimethylamino)propan-2-yl)-4-(trifluoromethoxy)benzenesulfonamide

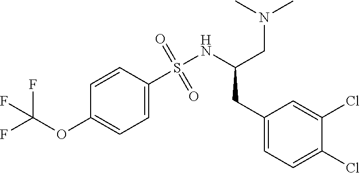

In a sealed vial under nitrogen, 4-(trifluoromethoxy)benzenesulfonyl chloride (0.029 mL, 0.169 mmol) was added to a stirred solution of (2R)-3-(3,4-dichlorophenyl)-N₁,N₁-dimethyl-propane-1,2-diamine;dihydrochloride (90%, 60 mg, 0.169 mmol) and triethylamine (0.094 mL, 0.675 mmol) in DCM (1.687 mL). The solution was stirred at rt overnight, diluted with an half saturated solution of NaHCO₃ and DCM, and the aqueous layer was extracted twice with DCM. The combined organic layers were filtered through phase separator then concentrated in vacuo and purified by flash chromatography on silica gel using a gradient of MeOH (0.7 N NH₃) in DCM from 0.4% to 4%. Desired fraction were concentrated in vacuo, and 1 M hydrogen chloride in diethyl ether (4.0 mL, 4.05 mmol) was added, the mixture was stirred 6 hours at rt, filtered, the solid was washed with diethyl ether and dried under vacuum at 50° C. overnight to afford the title compound as a white powder (60.3 mg, 97.8% purity, 70% yield, tᵣ=1.57 min). LCMS (Method C): m/z found 471 [M+H]⁺; ¹H-NMR (DMSO-d₆, 500 MHz) δ (ppm) 9.78 (br s, 1H), 8.44 (br d, J=8.6 Hz, 1H), 7.65 (d, J=7.8 Hz, 2H), 7.30-7.36 (m, 3H), 7.27 (d, J=8.3 Hz, 1H), 7.07 (dd, J=8.3, 2.0 Hz, 1H), 3.72-3.85 (m, 1H), 3.14-3.29 (m, 2H), 2.93 (dd, J=13.8, 4.5 Hz, 1H), 2.87 (br d, J=4.2 Hz, 6H), 2.41 (dd, J=13.8, 9.7 Hz, 1H).

### Example 124: (S)—N-(1-(3,4-dichlorophenyl)-3-(dimethylamino)propan-2-yl)-4-(trifluoromethoxy)benzenesulfonamide (247)

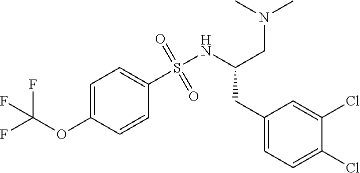

### Step 1: Synthesis of tert-butyl N-[(1S)-1-[(3,4-dichlorophenyl)methyl]-2-(dimethylamino)-2-oxo-ethyl]carbamate

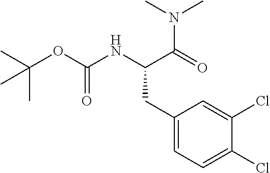

In a sealed vial under nitrogen, to a stirred solution of N-(tert-butoxycarbonyl)-3,4-dichloro-L-phenylalanine (97%, 1000 mg, 2.90 mmol) and [dimethylamino(triazolo[4,5-b]pyridin-3-yloxy)methylene]-dimethyl-ammonium hexafluorophosphate (1.435 g, 3.77 mmol) in DCM (13.193 mL) was added 2 M dimethylamine in THF (4.4 mL, 8.71 mmol). The reaction was stirred at rt for 10 minutes then N-ethyl-N-isopropyl-propan-2-amine (0.76 mL, 4.35 mmol) was added and the suspension was stirred overnight at rt. The resulting solution was diluted with an half saturated solution of NaHCO₃, and the aqueous layer was extracted twice with DCM. The organic layers were dried over phase separator, concentrated in vacuo and purified by flash chromatography on silica gel using a gradient of MeOH (0.7 N NH₃) in DCM from 0.4% to 4% to afford the title compound as a pale yellow oil. Remaining material (1 g) was purified by flash chromatography on silica gel using a gradient of EtOAc in Heptane from 25% to 75% to afford the title compound as a colorless oil (555 mg, 56.8%, tᵣ=0.93 min). LCMS (Method E): m/z found 305.1 [M+H-tBu]⁺; ¹H-NMR (DMSO-d₆, 400 MHz): δ (ppm) 7.62-7.49 (m, 2H), 7.26 (dd, J=8.3, 1.9 Hz, 1H), 7.08 (d, J=8.8 Hz, 1H), 4.56 (td, J=9.3, 5.1 Hz, 1H), 2.97 (s, 3H), 2.87 (dd, J=13.6, 5.0 Hz, 1H), 2.82 (s, 3H), 2.75 (dd, J=13.5, 9.7 Hz, 1H), 1.25 (s, 9H).

### Step 2: Synthesis of tert-butyl N-[(1S)-1-[(3,4-dichlorophenyl)methyl]-2-(dimethylamino)ethyl]carbamate

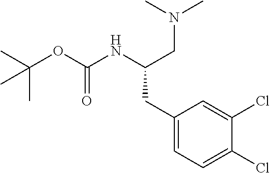

In a round bottomed flask, to a stirred solution of tert-butyl N-[(1S)-1-[(3,4-dichlorophenyl)methyl]-2-(dimethylamino)-2-oxo-ethyl]carbamate (500 mg, 1.38 mmol) in anhydrous THF (13.84 mL) at 0° C. under nitrogen was added 2.4 M lithium aluminum hydride (LAH) in THF (2.3 mL, 5.54 mmol) dropwise. The mixture was stirred at rt for 2 hours, quenched with water (15 μL, same weight as LAH used, dropwise addition), 15% aqueous sodium hydroxide solution and water (three times the weight of LAH used) were added. The resulting heterogeneous mixture was stirred at room temperature overnight, then dried over Na₂SO₄, filtered and concentrated under reduced pressure. The crude was purified by flash chromatography on silica gel using a gradient of methanol (0.7 N NH₃) in DCM from 1% to 8% to afford the title compound as a white powder (240 mg, 50% yield, tᵣ=0.66 min). LCMS (Method F): m/z found 347.3 [M+H]⁺; ¹H-NMR (DMSO-d₆, 400 MHz): δ (ppm) 7.51 (d, J=8.2 Hz, 1H), 7.42 (s, 1H), 7.17 (dd, J=8.2, 2.0 Hz, 1H), 6.82-6.12 (m, 1H), 3.77-3.55 (m, 1H), 2.87 (dd, J=13.6, 3.9 Hz, 1H), 2.50-2.35 (m, 1H), 2.20 (d, J=7.3 Hz, 2H), 2.16 (s, 6H), 1.29 (s, 9H).

### Step 3: Synthesis of (2S)-3-(3,4-dichlorophenyl)-N1,N1-dimethyl-propane-1,2-diamine

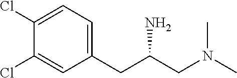

In a round-bottomed flask, to a stirred solution of tert-butyl N-[(1S)-1-[(3,4-dichlorophenyl)methyl]-2-(dimethylamino)ethyl]carbamate (264 mg, 0.760 mmol) in 1,4-dioxane (3.801 mL) at rt was added 4 M hydrogen chloride in dioxane (3.8 mL, 15.2 mmol). The reaction mixture was stirred at rt overnight, then filtered and washed with diethyl ether, to afford the title compound as a white powder (250.4 mg, 98% yield, tᵣ=0.45 min). LCMS (Method F): m/z found 247.2 [M+H]⁺; ¹H-NMR (DMSO-d₆, 400 MHz): δ (ppm) 10.80 (s, 1H), 8.51 (s, 3H), 7.70 (s, 1H), 7.64 (d, J=8.2 Hz, 1H), 7.37 (d, J=8.4 Hz, 1H), 4.04 (s, 1H), 3.38-3.10 (m, 2H), 3.02 (s, 2H), 2.81 (s, 6H)

### Step 4: Synthesis of (S)—N-(1-(3,4-dichlorophenyl)-3-(dimethylamino)propan-2-yl)-4-(trifluoromethoxy)benzenesulfonamide

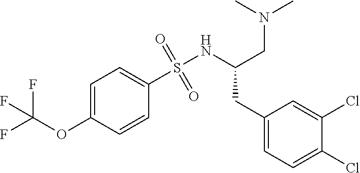

In a sealed vial under nitrogen, 4-(trifluoromethoxy)benzenesulfonyl chloride (0.040 mL, 0.238 mmol) was added to a stirred solution of (2S)-3-(3,4-dichlorophenyl)-N¹,N¹-dimethyl-propane-1,2-diamine;dihydrochloride (95%, 80 mg, 0.238 mmol) and triethylamine (0.13 mL, 0.951 mmol) in DCM (2.3768 mL). The solution was stirred at rt overnight, diluted with an half saturated solution of NaHCO₃ and DCM, and the aqueous layer was extracted twice with DCM. The combined organic layers were filtered through phase separator then concentrated in vacuo and purified by flash chromatography on silica gel using a gradient of MeOH (0.7 N NH₃) in DCM from 1% to 5%. Desired fraction were concentrated in vacuo, and 1 M hydrogen chloride in diethyl ether (4.8 mL, 4.75 mmol) was added, the mixture was stirred overnight at rt, filtered, the solid was washed with diethyl ether and dried under vacuum at 50° C. for 6 hours to afford the title compound as a white powder (99.9 mg, 99.7% purity, 82.5% yield, tᵣ=1.56 min). LCMS (Method C): m/z found 471 [M+H]⁺; ¹H-NMR (DMSO-d₆, 500 MHz) δ (ppm) 10.41-9.45 (m, 1H), 8.21 (d, J=8.6 Hz, 1H), 7.64 (d, J=8.8 Hz, 2H), 7.37-7.30 (m, J=2.0 Hz, 3H), 7.27 (d, J=8.1 Hz, 1H), 7.08 (dd, J=8.3, 2.2 Hz, 1H), 3.85-3.70 (m, 1H), 3.28-3.13 (m, 2H), 3.02-2.93 (m, 1H), 2.93-2.74 (m, J=3.4 Hz, 6H), 2.47-2.38 (m, 1H).

### Example 125: 4-cyclobutoxy-N-(1-(3,4-dichlorophenyl)-2-(dimethylamino)ethyl)benzenesulfonamide (248)

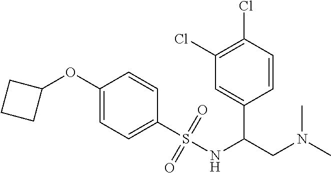 [SMILES: CN(C)CC(NS(=O)(=O)c1ccc(OC2CCC2)cc1)c1ccc(Cl)c(Cl)c1]

In a sealed vial under nitrogen, to a stirred solution of potassium iodide (20 mg, 0.122 mmol) and N-[1-(3,4-dichlorophenyl)-2-(dimethylamino)ethyl]-4-hydroxy-benzenesulfonamide (95%, 100 mg, 0.244 mmol) in anhydrous DMF (2.5 mL) was added bromocyclobutane (98%, 141 μL, 1.46 mmol) and dipotassium carbonate (202 mg, 1.46 mmol). The mixture was stirred at 80° C. for 18 h. Water (15 mL) was added and the aqueous layer was extracted with ethyl acetate (2×16 mL). The combined organic layers were washed with a saturated aqueous solution of sodium chloride, dried over sodium sulfate, filtered and concentrated under reduced pressure. The crude was purified by flash chromatography on silica gel using a gradient of methanol in dichloromethane from 0% to 5%. The desired fractions were combined and concentrated to afford the title compound as a yellow oil with impurities at 0.75 min (5%) and 0.80 min (9%). The product was further purified by reverse-phase chromatography (column C18 Aq 50 g) using a gradient of acetonitrile (+1% AcOH) in water (+1% AcOH) from 0% to 100%. The desired fractions were combined and concentrated. The product was diluted with a saturated aqueous solution of sodium bicarbonate and the aqueous layer was extracted (EtOAc×3). The combined organic layers were dried over sodium sulfate, filtered and concentrated under reduced pressure to afford the title compound as an off-white solid (46.1 mg, 100% purity, 42.606% yield, tᵣ=1.51 min). LCMS (Method C): m/z found 443.3; ¹H-NMR (DMSO-d₆, 500 MHz) δ (ppm) 7.92 (br s, 1H), 7.45-7.49 (m, J=9.0 Hz, 2H), 7.38 (d, J=8.3 Hz, 1H), 7.31 (d, J=2.0 Hz, 1H), 7.14 (dd, J=8.3, 2.0 Hz, 1H), 6.77-6.80 (m, J=9.0 Hz, 2H), 4.69 (quin, J=7.1 Hz, 1H), 4.27 (br t, J=7.3 Hz, 1H), 2.39-2.46 (m, 2H), 2.26-2.39 (m, 2H), 2.04 (s, 6H), 1.98-2.03 (m, 2H), 1.76-1.84 (m, 1H), 1.60-1.70 (m, 1H).

### Example 126: (S)-3-(3,4-dichlorophenyl)-N,N-dimethyl-2-((4-(trifluoromethoxy)phenyl)sulfonamido)propenamide (249)

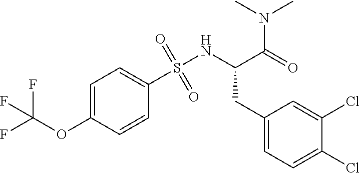

### Step 1: Synthesis of tert-butyl N-[(1S)-1-[(3,4-dichlorophenyl)methyl]-2-(dimethylamino)-2-oxo-ethyl]carbamate

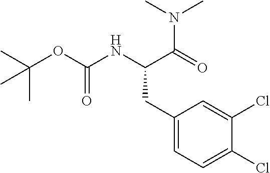

In a sealed vial under nitrogen, to a stirred solution of N-(tert-butoxycarbonyl)-3,4-dichloro-L-phenylalanine (97%, 1000 mg, 2.90 mmol) and [dimethylamino(triazolo[4,5-b]pyridin-3-yloxy)methylene]-dimethyl-ammonium hexafluorophosphate (1435 mg, 3.77 mmol) in DCM (13.193 mL) was added 2 M dimethylamine in THF (4.4 mL, 8.71 mmol). The reaction was stirred at rt for 10 minutes then N-ethyl-N-isopropyl-propan-2-amine (0.76 mL, 4.35 mmol) was added and the suspension was stirred overnight at rt. The resulting solution was diluted with an half saturated solution of NaHCO₃, and the aqueous layer was extracted twice with DCM. The organic layers were dried over phase separator, concentrated in vacuo and purified by flash chromatography on silica gel using a gradient of MeOH (0.7 N NH₃) in DCM from 0.4% to 4% to afford the title compound as a pale yellow oil. Material (1 g) was further purified by flash chromatography on silica gel using a gradient of EtOAc in Heptane from 25% to 75% to afford the title compound as a colorless oil (555 mg, 56.8% yield, tᵣ=0.93 min). LCMS (Method E): m/z found 305.1 [M+H-tBu]⁺; ¹H-NMR (DMSO-d₆, 400 MHz): δ (ppm) 7.62-7.49 (m, 2H), 7.26 (dd, J=8.3, 1.9 Hz, 1H), 7.08 (d, J=8.8 Hz, 1H), 4.56 (td, J=9.3, 5.1 Hz, 1H), 2.97 (s, 3H), 2.87 (dd, J=13.6, 5.0 Hz, 1H), 2.82 (s, 3H), 2.75 (dd, J=13.5, 9.7 Hz, 1H), 1.25 (s, 9H).

### Step 2: Synthesis of (2S)-2-amino-3-(3,4-dichlorophenyl)-N,N-dimethyl-propanamide

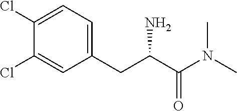 [SMILES: CN(C)C(=O)[C@@H](N)Cc1ccc(Cl)c(Cl)c1]

In a round-bottomed flask, to a stirred solution of tert-butyl N-[(1S)-1-[(3,4-dichlorophenyl)methyl]-2-(dimethylamino)-2-oxo-ethyl]carbamate (92%, 200 mg, 0.509 mmol) in 1,4-dioxane (2.5466 mL) at rt was added 4 M hydrogen chloride in dioxane (2.5 mL, 10.2 mmol). The mixture was stirred overnight at room temperature, then quenched with 25 mL of an half saturated solution of NaHCO₃. The aqueous layer was extracted twice with DCM, the combined organic layers were filtered through phase separator and concentrated in vacuo to afford the title compound as a pale yellow oil (125 mg, 86% yield, tᵣ=0.53 min). LCMS (Method F): m/z found 261.2 [M+H]⁺; ¹H-NMR (DMSO-d₆, 400 MHz): δ (ppm) 7.54-7.49 (m, 2H), 7.22 (dd, J=8.2, 2.0 Hz, 1H), 3.85 (dd, J=7.9, 5.7 Hz, 1H), 2.93 (s, 3H), 2.80 (s, 3H), 2.80-2.74 (m, 1H), 2.59 (dd, J=13.3, 7.9 Hz, 1H), 1.67 (s, 2H).

### Step 3: Synthesis of (S)-3-(3,4-dichlorophenyl)-N,N-dimethyl-2-((4-(trifluoromethoxy)phenyl)sulfonamido)propenamide

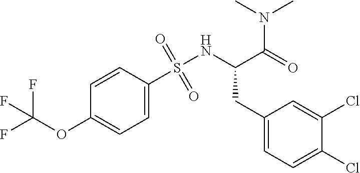

In a round bottomed flask under nitrogen 4-(trifluoromethoxy)benzenesulfonyl chloride (0.075 mL, 0.440 mmol) was added to a stirred solution of (2S)-2-amino-3-(3,4-dichlorophenyl)-N,N-dimethyl-propanamide (115 mg, 0.440 mmol) and triethylamine (0.25 mL, 1.76 mmol) in DCM (2.9357 mL). The solution was stirred at rt overnight then DCM and an half saturated solution of NaHCO₃ were added and the aqueous layer was extracted twice with DCM. The combined organic layers were filtered through phase separator, then concentrated in vacuo and purified by flash chromatography on silica gel using a gradient of MeOH in DCM from 0% to 5% to afford the title compound as a white powder (95.7 mg, 100% purity, 45% yield, tᵣ=2.55 min). LCMS (Method C): m/z found 485.2 [M+H]⁺; ¹H-NMR (DMSO-d₆, 500 MHz): δ (ppm) 8.35 (br s, 1H), 7.69 (d, J=8.4 Hz, 2H), 7.42-7.46 (m, 4H), 7.18 (dd, J=8.3, 2.0 Hz, 1H), 4.37 (br s, 1H), 2.90 (s, 3H), 2.83 (dd, J=13.7, 5.1 Hz, 1H), 2.67 (dd, J=13.7, 9.5 Hz, 1H), 2.56 (s, 3H).

### Example 127: N-(1-(3,4-dichlorophenyl)-2-(dimethylamino)ethyl)-N-methyl-4-(trifluoromethoxy)benzenesulfonamide (250)

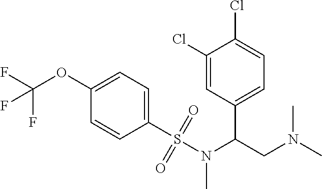 [SMILES: CN(C)CC(c1ccc(Cl)c(Cl)c1)N(C)S(=O)(=O)c1ccc(OC(F)(F)F)cc1]

In a sealed tube under nitrogen, a solution of N-[1-(3,4-dichlorophenyl)-2-(dimethylamino)ethyl]-4-(trifluoromethoxy)benzenesulfonamide (100 mg, 0.219 mmol) in dry THF (2 mL) was stirred at 0° C. Dry MeOH (13 μL, 0.328 mmol), triphenylphosphine (86 mg, 0.328 mmol) then diisopropyl azodicarboxylate (65 μL, 0.328 mmol) were added and the mixture was stirred at rt for 16 h. Half saturated aqueous NaHCO₃ was added and the mixture was extracted twice with EtOAc. The combined organic layers were washed with brine, dried over sodium sulfate, filtered and concentrated. The crude was purified by flash chromatography on silica gel using a gradient of (MeOH+2% NH₄OH) in DCM from 0.4% to 5%. The desired fractions were concentrated and the residue was dissolved in Et₂O. The solution was added dropwise to stirred 2 N HCl in Et₂O and the obtained suspension was stirred at rt for 4 h and filtered. The residue was washed with Et₂O and dried under reduced pressure at 60° C. for 16 h to afford the title compound as a white powder (83 mg, 98.6% purity, 73% yield, tᵣ=1.58 min). LCMS (Method C): m/z found 471.1 [M+H]⁺; ¹H-NMR (DMSO-d₆, 500 MHz): δ (ppm) 9.40-9.68 (m, 1H), 7.90 (d, J=8.8 Hz, 2H), 7.50 (br d, J=8.3 Hz, 2H), 7.47 (d, J=8.3 Hz, 1H), 7.28 (s, 1H), 7.15 (br d, J=8.3 Hz, 1H), 5.41 (br dd, J=11.1, 4.3 Hz, 1H), 4.09 (br t, J=11.5 Hz, 1H), 3.35-3.41 (m, 1H), 2.96 (br d, J=4.2 Hz, 3H), 2.89 (br d, J=4.4 Hz, 3H), 2.84 (s, 3H).

### Example 128: 4-(cyclopentylamino)-N-(1-(3,4-dichlorophenyl)-2-(dimethylamino)ethyl)benzenesulfonamide (251)

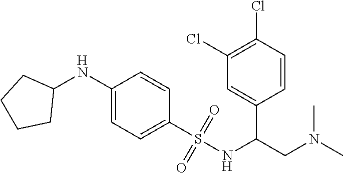

In a sealed vial, to a stirred solution of 4-bromo-N-[1-(3,4-dichlorophenyl)-2-(dimethylamino)ethyl]benzenesulfonamide (150 mg, 0.332 mmol) in anhydrous toluene (3 mL) at rt under nitrogen were added successively cyclopentanamine (49 μL, 0.498 mmol), Pd₂dba₃ (95%, 128 mg, 0.133 mmol), dicyclohexyl-[3,6-dimethoxy-2-(2,4,6-triisopropylphenyl)phenyl]phosphane (98%, 218 mg, 0.398 mmol) and ? (55 mg, 0.498 mmol). The reaction mixture was stirred at 100° C. for 18 h. After completion, the reaction mixture was cooled to rt and concentrated under reduced pressure. The resulting residue wad dissolved in DCM, and the combined organic layers were washed three times with water, once with brine, dried using a phase separator and concentrated under reduced pressure. The crude was purified by flash chromatography on silica gel using a gradient of MeOH (0.7 M NH₃) in DCM from 0.5% to 3%. The desired fractions were combined and concentrated. This impure fraction was purified by reverse-phase preparative chromatography using a gradient of acetonitrile in water from 0% to 100% (0.1% acetic acid in water). The desired fractions were combined and concentrated. The resulting oil was solubilized in Et₂O and 2 M HCl (1.7 mL, 3.32 mmol) was added. The precipitate formed was filtered, washed with Et₂O and dried under vacuum at 45° C. for 24 h to afford the title compound as a white powder (34 mg, 98% purity, 21% yield, tᵣ=1.57 min). LCMS (Method C): m/z found 456 [M+H]⁺; ¹H-NMR (DMSO-d₆, 500 MHz) δ (ppm) 9.59 (br d, 1H, J=1.5 Hz), 8.20 (d, 1H, J=10.5 Hz), 7.42 (d, 1H, J=2.0 Hz), 7.37 (d, 1H, J=8.3 Hz), 7.22 (dd, 1H, J=2.1, 8.4 Hz), 7.17 (d, 2H, J=8.8 Hz), 6.4-6.6 (m, 1H), 6.32 (d, 2H, J=8.8 Hz), 4.69 (dt, 1H, J=4.0, 10.7 Hz), 3.5-3.9 (m, 1H), 3.1-3.3 (m, 2H), 2.8-2.9 (m, 6H), 1.8-2.0 (m, 2H), 1.5-1.7 (m, 4H), 1.3-1.5 (m, 2H).

### Example 129: 4-(cyclobutylamino)-N-(1-(3,4-dichlorophenyl)-2-(dimethylamino)ethyl)benzenesulfonamide (252)

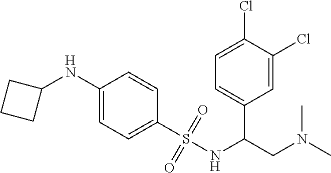

### Step 1: Synthesis of 4-bromo-N-[1-(3,4-dichlorophenyl)-2-(dimethylamino)ethyl]benzenesulfonamide

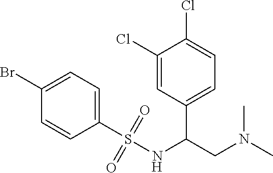

In a round-bottomed flask, to a stirred solution of triethylamine (0.80 mL, 5.75 mmol) in anhydrous DCM (17.738 mL) at r.t under nitrogen were added successively 4-bromobenzenesulfonyl chloride (98%, 499 mg, 1.91 mmol), and 1-(3,4-dichlorophenyl)-N²,N²-dimethylethane-1,2-diamine (100%, 446 mg, 1.91 mmol). The solution was stirred at rt overnight. The reaction mixture was diluted with water and the aqueous layer was extracted twice with DCM. The combined organic layers were washed with a saturated aqueous solution of NH₄Cl, a saturated aqueous solution of NaHCO₃ and a saturated aqueous solution of NaCl, dried over sodium sulfate, filtered and concentrated under reduced pressure. The crude was purified by flash chromatography on silica gel (column 24 g, 30 μm) using a gradient of MeOH in DCM from 0.5% to 5%. The desired fractions were concentrated to afford the title compound as a yellow solid (730 mg, 84.313% yield, tᵣ=0.67 min). LCMS (Method F): m/z found 453.1 [M+H]⁺; ¹H-NMR (DMSO-d₆, 400 MHz): δ (ppm) 8.24 (s, 1H), 7.63-7.56 (m, 2H), 7.52-7.46 (m, 2H), 7.40 (d, J=8.3 Hz, 1H), 7.30 (d, J=2.0 Hz, 1H), 7.15 (dd, J=8.3, 2.1 Hz, 1H), 5.75 (s, OH), 4.33 (t, J=7.4 Hz, 1H), 2.41 (dd, J=12.5, 8.2 Hz, 1H), 2.30 (dd, J=12.5, 6.9 Hz, 1H), 2.05 (s, 6H).

### Step 2: Synthesis of 4-(cyclobutylamino)-N-(1-(3,4-dichlorophenyl)-2-(dimethylamino)ethyl)benzenesulfonamide

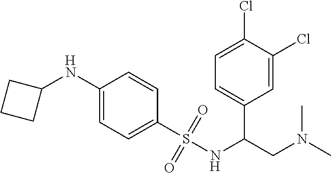

To a sealed tube, were added 4-bromo-N-[1-(3,4-dichlorophenyl)-2-(dimethylamino)ethyl]benzenesulfonamide (150 mg, 0.332 mmol), cyclobutanamine (98%, 36 mg, 0.498 mmol) in anhydrous toluene (7.5 mL) followed by dicyclohexyl-[3,6-dimethoxy-2-(2,4,6-triisopropylphenyl)phenyl]phosphane (98%, 109 mg, 0.199 mmol), sodium 2-methylbutan-2-olate (55 mg, 0.498 mmol) and cyclobutanamine (98%, 36 mg, 0.498 mmol). The reaction mixture was stirred at 100° C. for 48 h. Pd₂(dba)₃ (95%, 64 mg, 0.0663 mmol), dicyclohexyl-[3,6-dimethoxy-2-(2,4,6-triisopropylphenyl)phenyl]phosphane (98%, 109 mg, 0.199 mmol) and 2 M HCl (1.7 mL, 3.32 mmol) were added at rt and the reaction mixture was stirred at 100° C. for 24 h. After completion, the reaction mixture was cooled to rt and concentrated under reduced pressure. The resulting residue wad dissolved in DCM, and the combined organic layers were washed three times with water, once with brine, dried using a phase separator and concentrated under reduced pressure. The crude was purified by flash chromatography on silica gel using a gradient of MeOH (0.7 M NH₃) in DCM from 1.5% to 3%. The desired fractions were combined and concentrated. This impure fraction was purified by reverse-phase preparative chromatography using a gradient of acetonitrile in water from 0% to 100% (0.10% acetic acid in water). The desired fractions were combined and concentrated. The resulting oil was solubilized in Et₂O and 2 M HCl (1.7 mL, 3.32 mmol) was added. The precipitate formed was filtered, washed with Et₂O and dried under vacuum at 45° C. for 24 h to afford the title compound as a white powder (30 mg, 99.8% purity, 19% yield, tᵣ=1.45 min). LCMS (Method C): m/z found 442 [M+H]⁺; ¹H-NMR 1H NMR (DMSO-d₆, 500 MHz) δ (ppm) 9.56 (br d, 1H, J=1.7 Hz), 8.20 (d, 1H, J=10.5 Hz), 7.41 (d, 1H, J=2.0 Hz), 7.38 (d, 1H, J=8.3 Hz), 7.21 (dd, 1H, J=2.1, 8.4 Hz), 7.18 (d, 2H, J=8.8 Hz), 6.5-7.0 (m, 1H), 6.26 (d, 2H, J=9.0 Hz), 4.69 (dt, 1H, J=3.9, 10.6 Hz), 3.77 (quin, 1H, J=7.5 Hz), 3.2-3.3 (m, 1H), 3.17 (ddd, 1H, J=4.2, 8.3, 13.0 Hz), 2.8-2.9 (m, 6H), 2.2-2.3 (m, 2H), 1.5-1.9 (m, 4H).

### Example 130: N-((3-(3,4-dichlorophenyl)pyrrolidin-3-yl)methyl)-4-(trifluoromethoxy)benzenesulfonamide (253)

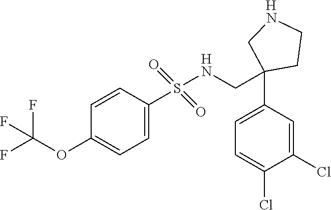

### Step 1: Synthesis of 1-(tert-butyl) 3-methyl 3-(3,4-dichlorophenyl)pyrrolidine-1,3-dicarboxylate

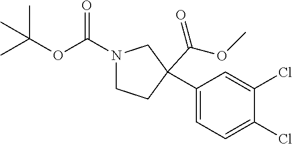 [SMILES: COC(=O)C1(c2ccc(Cl)c(Cl)c2)CCN(C(=O)OC(C)(C)C)C1]

In a round bottomed flask, to a stirred solution of methyl 3-(3,4-dichlorophenyl)pyrrolidine-3-carboxylate hydrochloride (200 mg, 0.644 mmol) and triethylamine (0.36 mL, 2.58 mmol) in DCM (3.2196 mL) was added di-tert-butyl dicarbonate (141 mg, 0.644 mmol). The reaction mixture was stirred at rt overnight. Water and DCM were added, and the aqueous layer was extracted twice with DCM. Organic layers were washed with a saturated solution of NH₄Cl, then with a saturated solution of NaHCO₃, then filtered through phase separator and concentrated in vacuo to afford the title compound as a colorless oil (240 mg, 87% purity, 87% yield, tᵣ=1.04 min). LCMS (Method E): m/z found 318.2 [M+H-tBu]⁺; ¹H-NMR (DMSO-d₆, 400 MHz): δ (ppm) 7.64 (d, J=8.4 Hz, 1H), 7.57 (d, J=2.2 Hz, 1H), 7.33 (dd, J=8.4, 2.2 Hz, 1H), 4.19 (d, J=10.9 Hz, 1H), 3.62 (s, 3H), 3.47 (d, J=10.8 Hz, 1H), 3.34 (s, 1H), 3.30-3.18 (m, 1H), 2.73 (d, J=9.4 Hz, 1H), 2.27 (d, J=8.9 Hz, 1H), 1.41 (d, J=8.2 Hz, 10H).

### Step 2: Synthesis of 1-tert-butoxycarbonyl-3-(3,4-dichlorophenyl)pyrrolidine-3-carboxylic acid

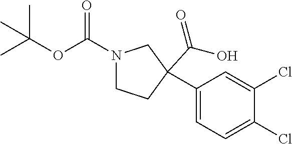 [SMILES: CC(C)(C)OC(=O)N1CCC(C(=O)O)(c2ccc(Cl)c(Cl)c2)C1]

In a round bottomed flask, to a stirred solution of 1-(tert-butyl) 3-methyl 3-(3,4-dichlorophenyl)pyrrolidine-1,3-dicarboxylate (87%, 190 mg, 0.442 mmol) in methanol (4.4167 mL) was added 0.5 M LiOH in water (1.8 mL, 0.883 mmol). The mixture was stirred at rt overnight, then 0.5 M hydrogen chloride in methanol (1.7 mL, 0.839 mmol) was added, and the mixture was brought to pH=4.5 using an aqueous solution of 1 N hydrogen chloride. The mixture was concentrated under reduced pressure to get rid of methanol, then the resulting suspension was filtered and the solid was washed with water and dried 2 hours under vacuum at 45° C. to afford the title compound as a white powder (140.2 mg, 100% purity, 88% yield, tᵣ=0.98 min). LCMS (Method D): m/z found 303.9 [M+H-tBu]⁺; ¹H-NMR (DMSO-d₆, 400 MHz): δ (ppm) 13.06 (s, 1H), 7.64 (d, J=8.4 Hz, 1H), 7.55 (d, J=2.2 Hz, 1H), 7.33 (dd, J=8.4, 2.2 Hz, 1H), 4.20 (d, J=10.6 Hz, 1H), 3.43-3.33 (m, 2H), 3.24 (dt, J=19.3, 9.5 Hz, 1H), 2.79-2.65 (m, 1H), 2.26-2.13 (m, 1H), 1.41 (d, J=7.6 Hz, 9H).

### Step 3: Synthesis of tert-butyl 3-(3,4-dichlorophenyl)-3-[[4-(trifluoromethoxy)phenyl]sulfonylcarbamoyl]pyrrolidine-1-carboxylate

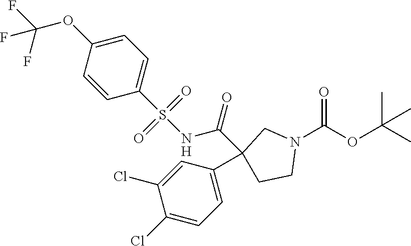

In a sealed vial, to a stirred solution of 4-(trifluoromethoxy)benzenesulfonamide (94 mg, 0.389 mmol) and 1-tert-butoxycarbonyl-3-(3,4-dichlorophenyl)pyrrolidine-3-carboxylic acid (100%, 140 mg, 0.389 mmol) in DCM (3.892 mL) was added 3-(ethyliminomethyleneamino)-N,N-dimethyl-propan-1-amine hydrochloride (95%, 157 mg, 0.778 mmol) and N,N-dimethylpyridin-4-amine (99%, 96 mg, 0.778 mmol). The reaction mixture was stirred at rt overnight, diluted with DCM and a half saturated solution of NH₄Cl. The aqueous layer was extracted twice with DCM, the organic layers were combined, filtered through phase separator concentrated in vacuo and purified by flash chromatography on silica gel using a gradient of MeOH in DCM from 2% to 10% to afford the title compound as a white powder (143 mg, 99% purity, 62% yield, tᵣ=1.08 min). LCMS (Method D): m/z found 527.1 [M+Na]⁺; ¹H-NMR (DMSO-d₆, 400 MHz): δ (ppm) 10.85 (s, 1H), 7.86 (t, J=8.8 Hz, 2H), 7.64-7.47 (m, 3H), 7.30 (d, J=8.8 Hz, 1H), 7.08 (t, J=6.3 Hz, 1H), 4.08 (t, J=10.9 Hz, 1H), 3.42 (dd, J=16.0, 11.3 Hz, 1H), 3.22 (ddd, J=10.9, 8.9, 3.2 Hz, 1H), 3.00 (dd, J=17.1, 9.6 Hz, 1H), 2.74-2.58 (m, 1H), 2.26-2.07 (m, 1H), 1.37 (d, J=17.1 Hz, 9H).

### Step 4: Synthesis of tert-butyl 3-(3,4-dichlorophenyl)-3-(((4-(trifluoromethoxy)phenyl)sulfonamido)methyl)pyrrolidine-1-carboxylate

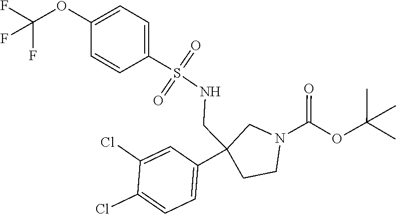 [SMILES: CC(C)(C)OC(=O)N1CCC(CNS(=O)(=O)c2ccc(OC(F)(F)F)cc2)(c2ccc(Cl)c(Cl)c2)C1]

In a sealed vial, to a stirred solution of tert-butyl 3-(3,4-dichlorophenyl)-3-[[4-(trifluoromethoxy)phenyl]sulfonylcarbamoyl]pyrrolidine-1-carboxylate (99%, 71 mg, 0.120 mmol) in anhydrous THF (1.2048 mL) at 0° C. under nitrogen was added 1 M LiAlH₄ in THF (0.36 mL, 0.361 mmol). The reaction mixture was stirred at 0° C. for 4 hours, then warmed slowly to rt and stirred at this temperature overnight. The mixture was quenched with a slow addition of water (5 mL), DCM (10 mL) and a saturated solution of Rochelle's salt (5 mL). The mixture was stirred 1 hour at rt, then extracted 4 times with DCM. The organic layers were concentrated in vacuo. The crude was purified by flash chromatography on silica gel using a gradient of MeOH (0.7 N NH₃) in DCM from 0.4% to 4% to afford the title compound as an off-white solid (25 mg, 66% purity, 24% yield, tᵣ=1.14 min). LCMS (Method D): m/z found 591.2 [M+Na]⁺; ¹H-NMR (DMSO-d₆, 400 MHz): δ (ppm) 7.95 (d, J=8.8 Hz, 1H), 7.80 (d, J=8.7 Hz, 2H), 7.70 (s, 1H), 7.55 (td, J=17.6, 15.8, 8.8 Hz, 4H), 7.45-7.17 (m, 2H), 3.71-3.59 (m, 1H), 3.41-3.32 (m, 2H), 3.30-3.20 (m, 2H), 3.11-2.93 (m, 2H), 2.33-2.21 (m, 1H), 2.07-1.95 (m, 1H), 1.40 (d, J=8.0 Hz, 9H).

### Step 5: Synthesis of N-((3-(3,4-dichlorophenyl)pyrrolidin-3-yl)methyl)-4-(trifluoromethoxy)benzenesulfonamide (253)

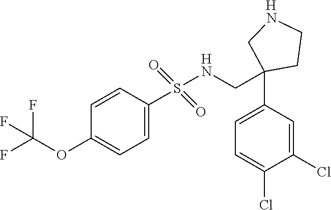

In a round-bottomed flask, to a stirred solution of tert-butyl 3-(3,4-dichlorophenyl)-3-[[[4-(trifluoromethoxy)phenyl]sulfonylamino]methyl]pyrrolidine-1-carboxylate (66%, 40 mg, 0.0464 mmol) in Diethyl ether (0.4636 mL) at rt was added 4 M hydrogen chloride in dioxane (0.46 mL, 1.85 mmol). The mixture was stirred overnight at rt, Then DCM and an half saturated aqueous solution of NaHCO₃ were added and the aqueous layer was extracted twice with DCM. The combined organic layers were filtered through phase separator, concentrated in vacuo. The crude was purified by reverse-phase chromatography using a gradient of acetonitrile in water from 0% to 100% (0.1% TFA). Desired fractions were gathered, concentrated in vacuo. DCM and an half saturated solution of NaHCO₃ were added, the aqueous layer was extracted twice with DCM. The combined organic layers were filtered through phase separator and concentrated in vacuo. The pale yellow oil obtained was solubilized in DCM (1 mL), and 2 M hydrogen chloride in diethyl ether (1.4 mL, 2.78 mmol) was added. The mixture was stirred overnight at rt, filtered and dried overnight under vacuum at 45° C. to afford the title compound as a white powder (23 mg, 96.7% purity, 95% yield, tᵣ=1.49 min). LCMS (Method C): m/z found 469 [M+H]⁺; ¹H-NMR (DMSO-d₆, 600 MHz) δ 8.9-9.2 (m, 2H), 7.82 (d, 3H, J=9.0 Hz), 7.58 (d, 1H, J=8.5 Hz), 7.5-7.6 (m, 3H), 7.27 (dd, 1H, J=2.3, 8.4 Hz), 3.57 (d, 1H, J=12.0 Hz), 3.2-3.4 (m, 3H), 3.0-3.2 (m, 2H), 2.3-2.5 (m, 1H), 2.16 (td, 1H, J=8.7, 13.2 Hz).

### Example 131: N-(1-(dimethylamino)propan-2-yl)-3-nitro-4-(trifluoromethoxy)benzenesulfonamide (254)

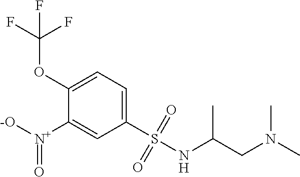

### Step 1: Synthesis of 3-nitro-4-(trifluoromethoxy)benzenesulfonyl chloride

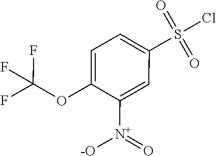

In a three necked round bottomed flask equipped with a thermometer, a solution of 3-nitro-4-(trifluoromethoxy)aniline (98%, 1.50 g, 6.62 mmol) in TFA (22.5 mL) was stirred at 0° C. copper(I) chloride (20 mg, 0.199 mmol) was added, the mixture was stirred at 0° C. for 5 min then a solution of sodium nitrite (579 mg, 8.27 mmol) in water (2.4 mL) was added dropwise over 20 min to keep the temperature below 0° C. The mixture was stirred at −5° C. for 30 min to afford Solution A. In a three necked round bottomed flask equipped with a thermometer, a solution of copper(II) dichloride (454 mg, 3.38 mmol) and concentrated hydrogen chloride (2.2 mL, 72.8 mmol) in AcOH (22.5 mL) and sulfurous acid (23 mL, 0.291 mol) was stirred at −5° C. Solution A was added dropwise over 20 min to keep the temperature below 5° C. The mixture was stirred at 0° C. for 5 min, then at rt for 1 h, and was extracted twice with DCM. The combined organic layers were dried over sodium sulfate, filtered and concentrated to afford the title compound as a brown oil (1.82 mg, 87% yield, tᵣ=0.78 min). LCMS (Method F); ¹H-NMR (DMSO-d₆, 500 MHz) δ 7.80 (d, 2H, J=8.8 Hz), 7.51 (br d, 3H, J=8.1 Hz), 7.3-7.3 (m, 2H), 7.2-7.3 (m, 2H), 2.87 (s, 2H), 1.94 (br dd, 2H, J=5.7, 11.4 Hz), 1.5-1.8 (m, 6H).

### Step 2: Synthesis of (R)—N-(1-(dimethylamino)propan-2-yl)-3-nitro-4-(trifluoromethoxy)benzenesulfonamide

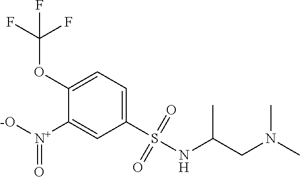

In a sealed vial under nitrogen, 3-nitro-4-(trifluoromethoxy)benzenesulfonyl chloride (75 mg, 0.245 mmol) was added to a stirred solution of N¹,N¹-dimethylpropane-1,2-diamine (25 mg, 0.245 mmol) and triethylamine (0.14 mL, 0.982 mmol) in DCM (2.454 mL). The solution was stirred at rt for 3 days, diluted with an half saturated solution of NaHCO₃ and DCM, and the aqueous layer was extracted twice with DCM. The combined organic layers were filtered through phase separator then concentrated in vacuo and purified by flash chromatography on silica gel using a gradient of MeOH in DCM from 2% to 5%. Desired fraction were concentrated in vacuo, and 1 M hydrogen chloride in diethyl ether (5.9 mL, 5.89 mmol) and methanol (2 mL) were added, the mixture was stirred overnight at rt, concentrated in vacuo and triturated in diethyl ether. The mixture was filtered, the solid was washed with diethyl ether and dried under vacuum at 50° C. overnight to afford the title compound as a brown powder (73.6 mg, 95.8% purity, 73.5% yield, tᵣ=0.95 min). LCMS (Method C): m/z found 372.2 [M+H]⁺; ¹H-NMR (DMSO-d₆, 500 MHz) δ (ppm) 9.60 (br s, 1H), 8.63 (d, J=2.2 Hz, 1H), 8.50 (br d, J=8.3 Hz, 1H), 8.32 (dd, J=8.7, 2.3 Hz, 1H), 8.02 (dd, J=8.7, 1.3 Hz, 1H), 3.72-3.89 (m, 1H), 3.00-3.16 (m, 2H), 2.79 (br s, 6H), 0.84 (d, J=6.6 Hz, 3H).

### Example 132: 2-(3,4-dichlorophenyl)-N,N-dimethyl-2-((4-(trifluoromethoxy)phenyl)sulfonamido)acetamide (255)

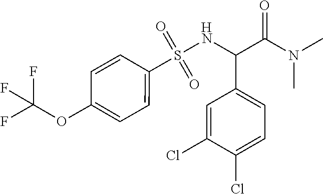

### Step 1: methyl (2R)-2-(3,4-dichlorophenyl)-2-[[4-(trifluoromethoxy)phenyl]sulfonylamino]acetate

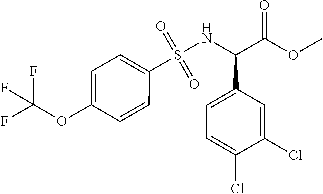

In a sealed tube under nitrogen, a suspension of 4-(trifluoromethoxy)benzenesulfonyl chloride (992 mg, 3.81 mmol) in DCM (15 mL) was stirred at 0° C. Methyl (2R)-2-amino-2-(3,4-dichlorophenyl)acetate hydrochloride (1.03 g, 3.81 mmol) was added and the mixture was stirred 5 min at 0° C. A solution of triethylamine (2.1 mL, 15.2 mmol) in DCM (3 mL) was added and the mixture was stirred at rt for 64 h. The mixture was diluted with DCM and half saturated aqueous NaHCO₃. The aqueous layer was extracted with DCM. The combined organic layers were washed with brine, dried over sodium sulfate, filtered and concentrated. The crude was purified by flash chromatography on silica gel using a gradient of (MeOH+2% NH₄OH) in DCM from 0.2% to 2% to afford the title compound as a yellow solid (550 mg, 96% purity, 30% yield, tᵣ=0.98 min). LCMS (Method F); ¹H-NMR (DMSO-d₆, 400 MHz): δ (ppm) 9.13 (s, 1H), 7.83-7.70 (m, 2H), 7.54-7.36 (m, 4H), 7.27 (dd, J=8.4, 2.1 Hz, 1H), 5.27 (s, 1H), 3.54 (s, 3H).

### Step 2: Synthesis of 2-(3,4-dichlorophenyl)-N,N-dimethyl-2-((4-(trifluoromethoxy)phenyl)sulfonamido)acetamide

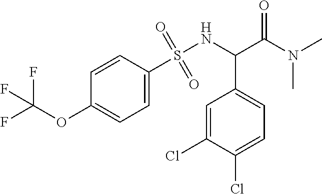 [SMILES: CN(C)C(=O)C(NS(=O)(=O)c1ccc(OC(F)(F)F)cc1)c1ccc(Cl)c(Cl)c1]

In a sealed tube under nitrogen, dichloro(dicyclopenta-2,4-dien-1-yl)zirconium (6.4 mg, 0.0218 mmol) was added to a stirred solution of 2 M dimethylamine in THF (2.7 mL, 5.46 mmol) and methyl (2R)-2-(3,4-dichlorophenyl)-2-[[4-(trifluoromethoxy)phenyl]sulfonylamino]acetate (100 mg, 0.218 mmol) in dry toluene (6 mL). The mixture was stirred at 40° C. for 20 h then was cooled to rt. Additional dichloro(dicyclopenta-2,4-dien-1-yl)zirconium (64 mg, 0.218 mmol) was added and the mixture was stirred at 45° C. for 20 h. The mixture was concentrated and the crude was purified by flash chromatography on silica gel using a gradient of (MeOH+2% NH₄OH) in DCM from 0.4% to 5% to afford the title compound as a yellow solid (58 mg, 96.4% purity, 54% yield, tᵣ=2.52 min). LCMS (Method C): m/z found 471 [M+H]⁺; ¹H-NMR (500 MHz, DMSO-d₆) δ 8.73 (d, 1H), J=9.8 Hz), 7.74 (d, 2H, J=8.8 Hz), 7.46 (d, 1H, J=2.0 Hz), 7.41 (d, 1H, J=8.3 Hz), 7.38 (d, 2H, J=8.3 Hz), 7.24 (dd, 1H, J=2.0, 8.3 Hz), 5.45 (d, 1H, J=9.8 Hz), 2.98 (s, 3H), 2.70 (s, 3H).

### Example 133: N-(1-(3,4-dichlorophenyl)-2-((4-methoxybenzyl)amino)ethyl)-4-(trifluoromethoxy)benzenesulfonamide (256)

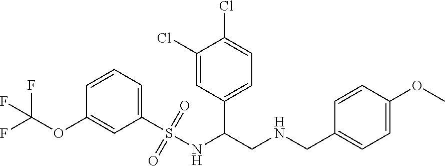 [SMILES: COc1ccc(CNCC(NS(=O)(=O)c2cccc(OC(F)(F)F)c2)c2ccc(Cl)c(Cl)c2)cc1]

### Step 1: Synthesis of methyl (2R)-2-(3,4-dichlorophenyl)-2-[[4-(trifluoromethoxy)phenyl]sulfonylamino]acetate

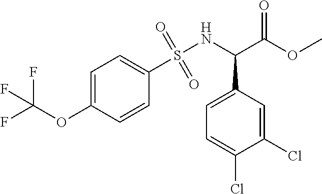 [SMILES: COC(=O)[C@H](NS(=O)(=O)c1ccc(OC(F)(F)F)cc1)c1ccc(Cl)c(Cl)c1]

In a sealed tube under nitrogen, A suspension of 4-(trifluoromethoxy)benzenesulfonyl chloride (992 mg, 3.81 mmol) in DCM (15 mL) was stirred at 0° C. Methyl (2R)-2-amino-2-(3,4-dichlorophenyl)acetate hydrochloride (1.03 g, 3.81 mmol) was added and the mixture was stirred 5 min at 0° C. A solution of triethylamine (2.1 mL, 15.2 mmol) in DCM (3 mL) was added and the mixture was stirred at rt for 64 h. The mixture was diluted with DCM and half saturated aqueous NaHCO₃. The aqueous layer was extracted with DCM. The combined organic layers were washed with brine, dried over sodium sulfate, filtered and concentrated. The crude was purified by flash chromatography on silica gel using a gradient of (MeOH+2% NH₄OH) in DCM from 0.2% to 2% to afford the title compound as a yellow solid (550 mg, 96% purity, 30% yield, tᵣ=0.98 min). LCMS (Method F); ¹H-NMR (DMSO-d₆, 400 MHz): δ (ppm) 9.13 (s, 1H), 7.83-7.70 (m, 2H), 7.54-7.36 (m, 4H), 7.27 (dd, J=8.4, 2.1 Hz, 1H), 5.27 (s, 1H), 3.54 (s, 3H).

### Step 2: Synthesis of 2-(3,4-dichlorophenyl)-N-[(4-methoxyphenyl)methyl]-2-[[4-(trifluoromethoxy)phenyl]sulfonylamino]acetamide

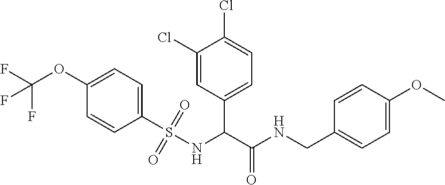 [SMILES: COc1ccc(CNC(=O)C(NS(=O)(=O)c2ccc(OC(F)(F)F)cc2)c2ccc(Cl)c(Cl)c2)cc1]

In a sealed tube under nitrogen, a solution of 4-methoxybenzylamine (1.0 mL, 7.91 mmol) in dry THF (2 mL) was added to a stirred solution of methyl (2R)-2-(3,4-dichlorophenyl)-2-[[4-(trifluoromethoxy)phenyl]sulfonylamino]acetate (145 mg, 0.316 mmol) and dichloro(dicyclopenta-2,4-dien-1-yl)zirconium (93 mg, 0.316 mmol) in dry toluene (3 mL). The mixture was stirred at 40° C. for 20 h then was cooled to rt. The mixture was concentrated and the crude was purified by flash chromatography on silica gel using a gradient of (MeOH+2% NH₄OH) in DCM from 1% to 6% to afford the title compound as a yellow solid (152 mg, 95% purity, 81% yield, tᵣ=0.98 min). LCMS (Method F): m/z found 563.2 [M+H]⁺; ¹H-NMR (DMSO-d₆, 400 MHz): δ (ppm) 8.91 (d, J=8.9 Hz, 1H), 8.70 (t, J=5.7 Hz, 1H), 7.81-7.67 (m, 2H), 7.51-7.31 (m, 4H), 7.24 (dd, J=8.4, 2.1 Hz, 1H), 7.06-6.94 (m, 2H), 6.92-6.72 (m, 2H), 5.09 (d, J=9.0 Hz, 1H), 4.20-3.95 (m, 2H), 3.72 (s, 3H).

### Step 3: Synthesis of N-(1-(3,4-dichlorophenyl)-2-((4-methoxybenzyl)amino)ethyl)-4-(trifluoromethoxy)benzenesulfonamide

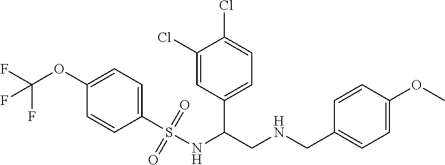 [SMILES: COc1ccc(CNCC(NS(=O)(=O)c2ccc(OC(F)(F)F)cc2)c2ccc(Cl)c(Cl)c2)cc1]

In a sealed tube under nitrogen, a solution of 2-(3,4-dichlorophenyl)-N-[(4-methoxyphenyl)methyl]-2-[[4-(trifluoromethoxy)phenyl]sulfonylamino]acetamide (150 mg, 0.266 mmol) in dry THF (5 mL) was stirred at 0° C. 2 M borane-methyl sulfide complex (399 μL, 0.799 mmol) was added dropwise and the mixture was stirred at rt for 2 h and at 60° C. for 16 h. The mixture was stirred at 0° C. and MeOH (600 μL) was added and the mixture was stirred at 0° C. for 30 min. 4 N aqueous HCl (450 μL) was added dropwise and the mixture was stirred at 60° C. for 1 h. The mixture was stirred at 0° C. and neutralized with aqueous 1N NaOH. The mixture was diluted with water and extracted twice with EtOAc. The combined organic layers were washed with brine, dried over sodium sulfate, filtered and concentrated. The crude was purified by flash chromatography on silica gel using a gradient of (MeOH+2% NH₄OH) in DCM from 1% to 6%. The desired fractions were concentrated and dissolved in Et₂O. The solution was added to stirred 2 N HCl in Et₂O. The mixture was stirred at rt for 16 h and the suspension was filtered. The residue was washed with Et₂O and dried under reduced pressure at 50° C. for 20 h to afford the title compound as a white powder (101 mg, 98.5% purity, 63% yield, tᵣ=1.76 min). LCMS (Method C): m/z found 549 [M+H]⁺; ¹H-NMR (DMSO-d₆, 500 MHz): δ (ppm) 9.02-9.36 (m, 2H), 8.81 (br d, J=8.8 Hz, 1H), 7.60 (d, J=8.8 Hz, 2H), 7.43 (br d, J=8.6 Hz, 2H), 7.36 (d, J=8.3 Hz, 1H), 7.25-7.32 (m, 3H), 7.09 (dd, J=8.3, 2.0 Hz, 1H), 6.99 (d, J=8.8 Hz, 2H), 4.72-4.87 (m, 1H), 4.10 (br s, 2H), 3.77 (s, 3H), 3.01-3.20 (m, 2H).

### Example 134: N-(1-(4-chlorophenyl)-2-(piperazin-1-yl)ethyl)-4-(trifluoromethoxy)benzenesulfonamide (257)

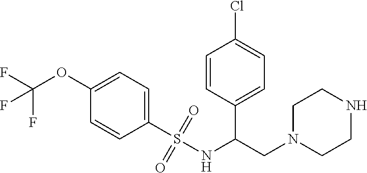 [SMILES: O=S(=O)(NC(CN1CCNCC1)c1ccc(Cl)cc1)c1ccc(OC(F)(F)F)cc1]

### Step 1: tert-butyl 4-[2-(4-chlorophenyl)-2-oxo-ethyl]piperazine-1-carboxylate

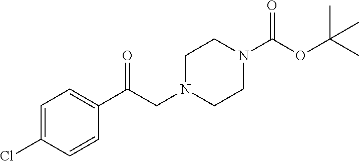 [SMILES: CC(C)(C)OC(=O)N1CCN(CC(=O)c2ccc(Cl)cc2)CC1]

In a sealed tube under nitrogen, a solution of 2-bromo-1-(4-chlorophenyl)ethanone (97%, 1.00 g, 4.15 mmol) and DMAP (25 mg, 0.208 mmol) in anhydrous DCM (11.131 mL) was stirred at rt. tert-butyl piperazine-1-carboxylate (97%, 957 mg, 4.99 mmol) then triethylamine (637 μL, 4.57 mmol) were added and the solution was stirred at rt for 16 h. The mixture was washed with a half saturated aqueous solution of NaHCO₃. The aqueous layer was extracted twice with DCM. The combined organic layers were filtered through phase separator and concentrated. The crude was purified by flash chromatography on silica gel using a gradient of MeOH in DCM from 2% to 10% to afford the title compound as a pale yellow oil (1474 mg, 90.5% purity, 94% yield, tᵣ=0.58 min). LCMS (Method F): m/z found 339.3 [M+H]⁺; ¹H-NMR (DMSO-d₆, 400 MHz): δ (ppm) 8.03-7.97 (m, 2H), 7.59 (d, J=8.5 Hz, 2H), 3.87 (s, 2H), 3.33 (s, 2H), 3.30 (s, 2H), 2.49-2.42 (m, 4H), 1.40 (s, 9H).

### Step 2: Synthesis of tert-butyl 4-(2-amino-2-(4-chlorophenyl)ethyl)piperazine-1-carboxylate

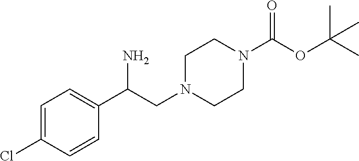 [SMILES: CC(C)(C)OC(=O)N1CCN(CC(N)c2ccc(Cl)cc2)CC1]

In a sealed tube under nitrogen, NaBH₃CN (3959 mg, 7.92 mmol) was added to a stirred solution of tert-butyl 4-[2-(4-chlorophenyl)-2-oxo-ethyl]piperazine-1-carboxylate (91%, 1474 mg, 3.96 mmol) and NH₄OAc (98%, 3114 mg, 39.6 mmol) in MeOH (13.196 mL). The mixture was stirred at 65° C. overnight, was concentrated and the residue was dissolved in DCM and filtered. The filtrate was concentrated in vacuo and purified by flash chromatography on silica gel using a gradient of MeOH (0.7 N NH₃) in DCM from 0.4% to 5%. Desired fractions were gathered, concentrated in vacuo then dissolved in DCM and an half saturated aqueous solution of NaHCO₃ was added and the aqueous layers was extracted with DCM. The combined organic layers were filtered through phase separator, concentrated in vacuo to afford the title compound as a yellow oil (832 mg, 97.10% purity, 60% yield, tᵣ=0.61 min). LCMS (Method F): m/z found 340.3 [M+H]⁺; ¹H-NMR (DMSO-d₆, 400 MHz): δ (ppm) 7.45-7.30 (m, 4H), 4.04 (dd, J=9.2, 4.8 Hz, 1H), 3.33-3.29 (m, 4H), 2.48-2.38 (m, 2H), 2.37-2.20 (m, 4H), 1.90 (s, 2H), 1.40 (s, 9H).

### Step 3: tert-butyl 4-[2-(4-chlorophenyl)-2-[[4-(trifluoromethoxy)phenyl]sulfonylamino]ethyl]piperazine-1-carboxylate

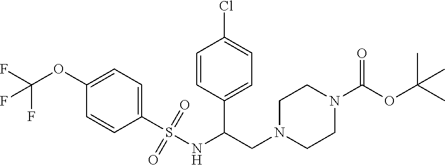 [SMILES: CC(C)(C)OC(=O)N1CCN(CC(NS(=O)(=O)c2ccc(OC(F)(F)F)cc2)c2ccc(Cl)cc2)CC1]

In a sealed vial under nitrogen, 4-(trifluoromethoxy)benzenesulfonyl chloride (0.050 mL, 0.294 mmol) was added to a stirred solution of tert-butyl 4-[2-amino-2-(4-chlorophenyl)ethyl]piperazine-1-carboxylate (100 mg, 0.294 mmol) and triethylamine (0.16 mL, 1.18 mmol) in DCM (2.9424 mL). The solution was stirred at rt overnight, diluted with a half saturated solution of NaHCO₃ and DCM, and the aqueous layer was extracted twice with DCM. The combined organic layers were filtered through phase separator then concentrated in vacuo and purified by flash chromatography on silica gel using a gradient of MeOH (0.7N NH₃) in DCM from 0.4% to 4%. to afford the title compound as a pale yellow oil (122 mg, 98.1% purity, 72% yield, tᵣ=0.77 min). LCMS (Method F): m/z found 564.2 [M+H]⁺; ¹H-NMR (DMSO-d₆, 400 MHz): δ (ppm) 8.31 (d, J=5.2 Hz, 1H), 7.88-7.72 (m, 2H), 7.45 (d, J=8.1 Hz, 2H), 7.30-7.16 (m, 4H), 4.37 (s, 1H), 3.17-2.94 (m, 4H), 2.49-2.45 (m, 1H), 2.32-2.15 (m, 5H), 1.37 (s, 9H).

### Step 4: Synthesis of N-[1-(4-chlorophenyl)-2-piperazin-1-yl-ethyl]-4-(trifluoromethoxy)benzenesulfonamide

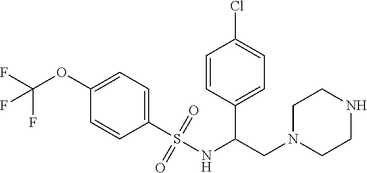 [SMILES: O=S(=O)(NC(CN1CCNCC1)c1ccc(Cl)cc1)c1ccc(OC(F)(F)F)cc1]

In a round-bottomed flask, to a stirred solution of tert-butyl 4-[2-(4-chlorophenyl)-2-[[4-(trifluoromethoxy)phenyl]sulfonylamino]ethyl]piperazine-1-carboxylate (123 mg, 0.218 mmol) in diethyl ether (2.1808 mL) at rt was added 4 M HCl (2.2 mL, 8.72 mmol). The mixture was stirred overnight at rt, diethyl ether (15 mL) was added, the mixture was stirred for 1 hour, then the solid was filtered and dried overnight at 60° C. to afford the title compound as a white powder (110.5 mg, 99.07% purity, 100% yield, tᵣ=1.44 min). LCMS (Method C): m/z found 464 [M+H]⁺; ¹H-NMR (400 MHz, DMSO-d₆) δ 9.85-9.09 (m, 1H), 9.10-8.88 (m, 1H), 8.85-8.15 (m, 1H), 7.66 (d, J=8.8 Hz, 2H), 7.34 (br d, J=8.5 Hz, 2H), 7.24-7.17 (m, 2H), 7.17-7.10 (m, 2H), 5.33-4.57 (m, 1H), 4.52-3.96 (m, 4H), 3.20-2.64 (m, 6H).

### Example 135: N-(1-(4-chloro-3-fluorophenyl)-2-(piperazin-1-yl)ethyl)-4-(trifluoromethoxy)benzenesulfonamide (258)

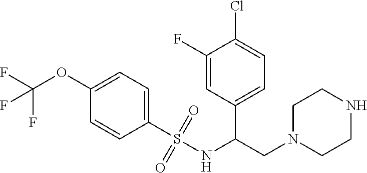 [SMILES: O=S(=O)(NC(CN1CCNCC1)c1ccc(Cl)c(F)c1)c1ccc(OC(F)(F)F)cc1]

### Step 1: Synthesis of tert-butyl 4-(2-(4-chloro-3-fluorophenyl)-2-oxoethyl)piperazine-1-carboxylate

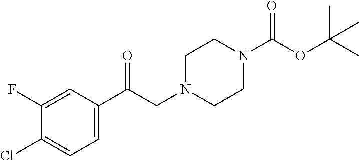 [SMILES: CC(C)(C)OC(=O)N1CCN(CC(=O)c2ccc(Cl)c(F)c2)CC1]

In a sealed tube under nitrogen, a solution of 3-fluoro-4-chlorophenacyl bromide (1.00 g, 3.98 mmol) and DMAP (24 mg, 0.199 mmol) in anhydrous DCM (10.65 mL) was stirred at rt. tert-butyl piperazine-1-carboxylate (97%, 916 mg, 4.77 mmol), then triethylamine (610 μL, 4.37 mmol), were added and the solution was stirred at rt for 16 h. The mixture was washed with a half saturated aqueous solution of NaHCO₃. The aqueous layer was extracted twice with DCM. The combined organic layers were filtered through phase separator and concentrated. The crude was purified by flash chromatography on silica gel using a gradient of MeOH (0.7N NH₃) in DCM from 0.4% to 4% to afford the title compound as a pale yellow solid (1305 mg, 84% purity, 84.4% yield, tᵣ=0.63 min). LCMS (Method E): m/z found 357.1 [M+H]⁺; ¹H-NMR (DMSO-d₆, 400 MHz) δ (ppm) 7.96 (dd, J=10.1, 1.9 Hz, 1H), 7.84 (dd, J=8.4, 1.8 Hz, 1H), 7.77 (dd, J=8.3, 7.4 Hz, 1H), 3.89 (s, 2H), 3.39-3.32 (m, 2H), 3.31-3.24 (m, 2H), 2.49-2.44 (m, 4H), 1.40 (s, 9H).

### Step 2: Synthesis of tert-butyl 4-(2-amino-2-(4-chloro-3-fluorophenyl)ethyl)piperazine-1-carboxylate

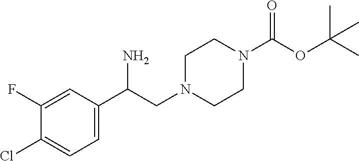 [SMILES: CC(C)(C)OC(=O)N1CCN(CC(N)c2ccc(Cl)c(F)c2)CC1]

In a sealed tube under nitrogen, NaBH₃CN (2.012 g, 4.02 mmol) was added to a stirred solution of tert-butyl 4-[2-(4-chloro-3-fluoro-phenyl)-2-oxo-ethyl]piperazine-1-carboxylate (718 mg, 2.01 mmol) and NH₄OAc (98%, 1583 mg, 20.1 mmol) in methanol (10.061 mL). The mixture was stirred at 65° C. overnight and filtered. The filtrate was concentrated in vacuo and purified by flash chromatography on silica gel using a gradient of MeOH (0.7 N NH₃) in DCM from 0.4% to 5%. Desired fractions were gathered, concentrated in vacuo, then dissolved in DCM, and an half saturated aqueous solution of NaHCO₃ was added. The aqueous layer was extracted with DCM. The combined organic layers were filtered through phase separator and concentrated in vacuo to afford the title compound as a yellow oil (424 mg, 98% purity, 58% yield, tᵣ=0.52 min). LCMS (Method F): m/z found 358.3 [M+H]⁺; ¹H-NMR (DMSO-d₆, 400 MHz) δ (ppm) 7.49 (t, J=8.0 Hz, 1H), 7.43 (dd, J=10.9, 1.9 Hz, 1H), 7.24 (dd, J=8.3, 1.8 Hz, 1H), 4.06 (dd, J=8.3, 5.6 Hz, 1H), 3.31-3.22 (m, 4H), 2.43 (dt, J=10.0, 4.6 Hz, 2H), 2.36-2.25 (m, 4H), 1.96 (s, 2H), 1.40 (s, 9H).

### Step 3: Synthesis of tert-butyl 4-(2-(4-chloro-3-fluorophenyl)-2-((4-(trifluoromethoxy)phenyl)sulfonamido)ethyl)piperazine-1-carboxylate

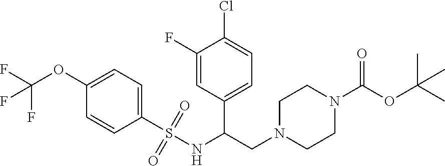 [SMILES: CC(C)(C)OC(=O)N1CCN(CC(NS(=O)(=O)c2ccc(OC(F)(F)F)cc2)c2ccc(Cl)c(F)c2)CC1]

In a sealed vial under nitrogen, 4-(trifluoromethoxy)benzenesulfonyl chloride (0.047 mL, 0.279 mmol) was added to a stirred solution of tert-butyl 4-[2-amino-2-(4-chloro-3-fluoro-phenyl)ethyl]piperazine-1-carboxylate (100 mg, 0.279 mmol) and triethylamine (0.16 mL, 1.12 mmol) in DCM (2.79 mL). The solution was stirred at rt overnight, diluted with a half saturated solution of NaHCO₃, then DCM was added. The aqueous layer was extracted twice with DCM. The combined organic layers were filtered through phase separator then concentrated in vacuo and purified by flash chromatography on silica gel using a gradient of MeOH (0.7 N NH₃) in DCM from 0.4% to 4% to afford the title compound as a white foam (80 mg, 91.3% purity, 45% yield, tᵣ=0.78 min). LCMS (Method E): m/z found 582.3 [M+H]⁺; ¹H-NMR (DMSO-d₆, 400 MHz) δ (ppm) 8.35 (s, 1H), 7.83-7.72 (m, 2H), 7.45 (d, J=8.0 Hz, 2H), 7.39 (t, J=8.0 Hz, 1H), 7.24-7.05 (m, 2H), 4.49-4.35 (m, 1H), 3.08 (d, J=17.8 Hz, 4H), 2.55-2.52 (m, 1H), 2.37-2.15 (m, 5H), 1.37 (s, 9H).

### Step 4: Synthesis of N-(1-(4-chloro-3-fluorophenyl)-2-(piperazin-1-yl)ethyl)-4-(trifluoromethoxy)benzenesulfonamide (258)

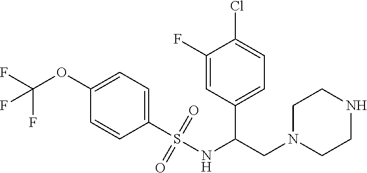 [SMILES: O=S(=O)(NC(CN1CCNCC1)c1ccc(Cl)c(F)c1)c1ccc(OC(F)(F)F)cc1]

In a round bottomed flask, to a stirred solution of tert-butyl 4-[2-(4-chloro-3-fluoro-phenyl)-2-[[4-(trifluoromethoxy)phenyl]sulfonylamino]ethyl]piperazine-1-carboxylate (91%, 80 mg, 0.125 mmol) in diethyl ether (1.26 mL) was added 4 M hydrogen chloride in dioxane (1.3 mL, 5.02 mmol). The mixture was stirred overnight at rt, filtered washed with Et₂O and dried overnight under vacuum at 45° C. to afford the HCl salt of the title compound as a white powder (65 mg, 98.7% purity, 99% yield, tᵣ=1.73 min). LCMS (Method C): m/z found 482 [M+H]⁺; ¹H-NMR (DMSO-d₆, 500 MHz): δ (ppm) 8.94-9.52 (br s, 2H), 8.78 (br s, 1H), 7.68 (d, J=8.8 Hz, 2H), 7.30-7.37 (m, 3H), 7.22 (dd, J=10.4, 1.6 Hz, 1H), 7.07 (dd, J=8.3, 1.7 Hz, 1H), 4.70 (br s, 1H), 2.70-3.48 (br s, 10H).

### Example 136: N-(1-(4-chlorophenyl)-2-(piperazin-1-yl)ethyl)-6-(trifluoromethoxy)pyridine-3-sulfonamide (259)

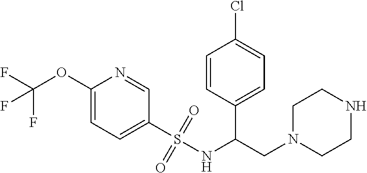 [SMILES: O=S(=O)(NC(CN1CCNCC1)c1ccc(Cl)cc1)c1ccc(OC(F)(F)F)nc1]

### Step 1: Synthesis of tert-butyl 4-(2-(4-chlorophenyl)-2-oxoethyl)piperazine-1-carboxylate

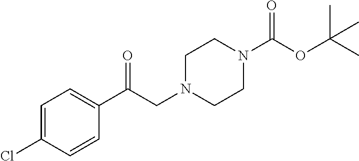 [SMILES: CC(C)(C)OC(=O)N1CCN(CC(=O)c2ccc(Cl)cc2)CC1]

In a sealed tube under nitrogen, a solution of 2-bromo-1-(4-chlorophenyl)ethanone (97%, 1.00 g, 4.15 mmol) and DMAP (25 mg, 0.208 mmol) in anhydrous DCM (11.13 mL) was stirred at rt, then tert-butyl piperazine-1-carboxylate (97%, 957 mg, 4.99 mmol), followed by triethylamine (637 μL, 4.57 mmol) were added and the solution was stirred at rt for 16 h. The mixture was washed with a half saturated aqueous solution of NaHCO₃. The aqueous layer was extracted twice with DCM. The combined organic layers were filtered through phase separator and concentrated. The crude was purified by flash chromatography on silica gel using a gradient of MeOH in DCM from 2% to 10% to afford the title compound as a pale yellow oil (1.474 g, 90.5% purity, 94.5% yield, tᵣ=0.58 min). LCMS (Method F): m/z found 339.3 [M+H]⁺; ¹H-NMR (DMSO-d₆, 400 MHz): δ (ppm) 8.03-7.97 (m, 2H), 7.59 (d, J=8.5 Hz, 2H), 3.87 (s, 2H), 3.33 (s, 2H), 3.30 (s, 2H), 2.49-2.42 (m, 4H), 1.40 (s, 9H).

### Step 2: Synthesis of tert-butyl 4-(2-amino-2-(4-chlorophenyl)ethyl)piperazine-1-carboxylate

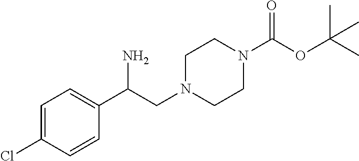 [SMILES: CC(C)(C)OC(=O)N1CCN(CC(N)c2ccc(Cl)cc2)CC1]

In a sealed tube under nitrogen, NaBH₃CN (3.96 g, 7.92 mmol) was added to a stirred solution of tert-butyl 4-[2-(4-chlorophenyl)-2-oxo-ethyl]piperazine-1-carboxylate (91%, 1.47 g, 3.96 mmol) and NH₄OAc (98%, 3.114 g, 39.6 mmol) in methanol (13.20 mL). The mixture was stirred at 65° C. overnight, then concentrated and the residue was dissolved in DCM and filtered. The filtrate was concentrated in vacuo and purified by flash chromatography on silica gel using a gradient of MeOH (0.7 N NH₃) in DCM from 0.4% to 5%. Desired fractions were gathered, concentrated in vacuo then dissolved in DCM. A half saturated aqueous solution of NaHCO₃ was added and the aqueous layer was extracted with DCM. The combined organic layers were filtered through a phase separator and concentrated in vacuo to afford the title compound as a yellow oil (832 mg, 97.1% purity, 60% yield, tᵣ=0.61 min). LCMS (Method F): m/z found 340.3 [M+H]⁺; ¹H-NMR (DMSO-d₆, 400 MHz): δ (ppm) 7.45-7.30 (m, 4H), 4.04 (dd, J=9.2, 4.8 Hz, 1H), 3.33-3.29 (m, 4H), 2.48-2.38 (m, 2H), 2.37-2.20 (m, 4H), 1.90 (s, 2H), 1.40 (s, 9H).

### Step 3: Synthesis of 5-benzylsulfanyl-2-(trifluoromethoxy)pyridine

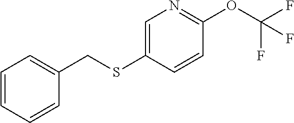 [SMILES: FC(F)(F)Oc1ccc(SCc2ccccc2)cn1]

A sealed vial was charged with 5-bromo-2-(trifluoromethoxy)pyridine (95%, 1.00 g, 3.93 mmol) and DIPEA (1.4 mL, 7.85 mmol) in anhydrous 1,4-dioxane (13.086 mL). The reaction mixture was degassed with argon for 5 min. Next, Xantphos (227 mg, 0.393 mmol), tris(dibenzylideneacetone)dipalladium(0) (180 mg, 0.196 mmol), and benzyl mercaptan (922 μL, 7.85 mmol) were added, and the reaction mixture was stirred at 105° C. for 3 hours. The mixture was cooled to rt, filtered on a pad of decalite, washed with dioxane and DCM, and concentrated. The crude product was purified by flash chromatography on silica gel using a gradient of EtOAc in heptane from 0 to 10% to afford the title compound as a pale yellow oil (1.072 g, 96% purity, 92% yield, tᵣ=1.03 min). LCMS (Method E): m/z found 286.1 [M+H]⁺; ¹H-NMR (DMSO-d₆, 400 MHz): δ (ppm) 8.30-8.25 (m, 1H), 7.99 (dd, J=8.6, 2.6 Hz, 1H), 7.36-7.21 (m, 6H), 4.31 (s, 2H).

### Step 4: Synthesis of tert-butyl 4-(2-(4-chlorophenyl)-2-((6-(trifluoromethoxy)pyridine)-3-sulfonamido)ethyl)piperazine-1-carboxylate

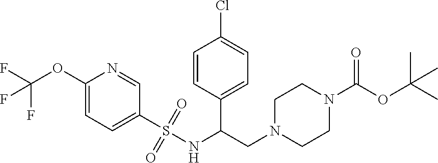 [SMILES: CC(C)(C)OC(=O)N1CCN(CC(NS(=O)(=O)c2ccc(OC(F)(F)F)nc2)c2ccc(Cl)cc2)CC1]

In a round-bottomed flask, to a stirred solution of 5-benzylsulfanyl-2-(trifluoromethoxy)pyridine (150 mg, 0.526 mmol) in acetonitrile (4.41 mL) at 0° C., under nitrogen, were added successively water (0.11 mL), acetic acid (0.17 mL), and then 1,3-dichloro-5,5-dimethyl-imidazolidine-2,4-dione (207 mg, 1.05 mmol). The reaction mixture was stirred at 0° C. for 2 h, then concentrated in vacuo. The residue was dissolved in DCM (5 mL) and quenched with 10 mL of 5% NaHCO₃ at 0° C. The organic layer was dried through phase separator and added dropwise to a solution of tert-butyl 4-[2-amino-2-(4-chlorophenyl)ethyl]piperazine-1-carboxylate (214 mg, 0.631 mmol) and triethylamine (0.081 mL, 0.578 mmol) in DCM (3 mL). The mixture was stirred overnight at rt. Next, a half saturated solution of NaHCO₃ was added, and the mixture was extracted twice with DCM. The combined organic layers were dried using phase separator, concentrated in vacuo, and purified by flash chromatography on silica gel using a gradient of methanol (0.7 N NH₃) in DCM from 0.4% to 2% to afford the title compound as an off-white powder (201.5 mg, 95% purity, 64% yield, tᵣ=0.75 min). LCMS (Method E): m/z found 565.3 [M+H]⁺; ¹H-NMR (DMSO-d₆, 400 MHz): δ (ppm) 8.60 (d, J=2.5 Hz, 2H), 8.19 (dd, J=8.6, 2.5 Hz, 1H), 7.37 (d, J=8.6 Hz, 1H), 7.28-7.20 (m, 4H), 4.46 (s, 1H), 3.18-2.94 (m, 4H), 2.56 (dd, J=13.1, 9.6 Hz, 1H), 2.33-2.20 (m, 5H), 1.38 (s, 9H).

### Step 5: Synthesis of N-(1-(4-chlorophenyl)-2-(piperazin-1-yl)ethyl)-6-(trifluoromethoxy)pyridine-3-sulfonamide (259)

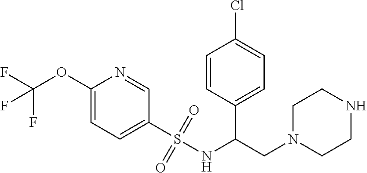 [SMILES: O=S(=O)(NC(CN1CCNCC1)c1ccc(Cl)cc1)c1ccc(OC(F)(F)F)nc1]

In a round bottomed flask, to a stirred solution of tert-butyl 4-[2-(4-chlorophenyl)-2-[[6-(trifluoromethoxy)-3-pyridyl]sulfonylamino]ethyl]piperazine-1-carboxylate (202 mg, 0.357 mmol) in diethyl ether (3.57 mL) was added 2 M hydrogen chloride in diethyl ether (3.6 mL, 7.13 mmol). The mixture was stirred for 3 h at rt, then 4 M hydrogen chloride in dioxane (2.2 mL, 8.92 mmol) was added and the mixture was stirred for an addition 3 h at rt. The mixture was filtered, washed with diethyl ether, and dried overnight at 50° C. The solid was purified by reverse-phase chromatography using a gradient of acetonitrile in water from 0% to 100% (0.1% TFA). The desired fractions were gathered and concentrated. Next, a half saturated solution of NaHCO₃ was added, and the aqueous solution was extracted twice with DCM. The organic layers were filtered through a phase separator, concentrated in vacuo. Next, 2 N HCl in diethyl ether was added, the mixture was stirred overnight at rt, concentrated in vacuo, and purified once more by reverse-phase chromatography using a gradient of acetonitrile in water from 0% to 100% (0.1% TFA). Desired fractions were gathered, concentrated in vacuo, then 2 M hydrogen chloride in diethyl ether (3.6 mL, 7.13 mmol) was added and the mixture was stirred 2 h at rt and filtered to afford the title compound as a white powder (30 mg, 99.6% purity, 18% yield, tᵣ=1.6 min). LCMS (Method C): m/z found 465 [M+H]⁺; ¹H-NMR (DMSO-d₆, 500 MHz) δ 8.5-9.3 (m, 3H), 8.46 (s, 1H), 8.09 (dd, 1H, J=2.4, 8.6 Hz), 7.2-7.4 (m, 5H), 4.4-4.8 (m, 1H), 2.5-3.3 (m, 10H).

### Example 137: N-((4-phenylpiperidin-4-yl)methyl)-4-(trifluoromethoxy)benzenesulfonamide (260)

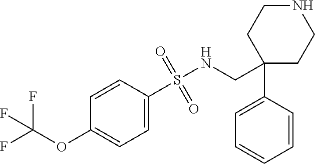 [SMILES: O=S(=O)(NCC1(c2ccccc2)CCNCC1)c1ccc(OC(F)(F)F)cc1]

### Step 1: Synthesis of 4-phenyl-1-tritylpiperidine-4-carbonitrile

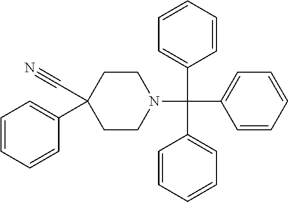 [SMILES: N#CC1(c2ccccc2)CCN(C(c2ccccc2)(c2ccccc2)c2ccccc2)CC1]

In a round-bottomed flask, to a stirred suspension of 4-phenylpiperidine-4-carbonitrile hydrochloride (1:1) (1.00 g, 4.49 mmol) in anhydrous DMF (15 mL) at room temperature was added triethylamine (1.9 mL, 13.6 mmol) and [chloro(diphenyl)methyl]benzene (1.50 g, 5.39 mmol). The reaction mixture was stirred for 3 h at this temperature, then diluted with water and dichloromethane. The layers were separated. The aqueous layer was extracted twice with ethyl acetate. The combined organic layers were washed once with water and once with a saturated aqueous solution of NaCl, then dried using a phase separator, and concentrated under reduced pressure. The residue was triturated in methanol, filtered, then washed with methanol, and dried under vacuum at room temperature for 18 h to afford the title compound as a white powder (1.83 g, 95% purity, 90% yield). ¹H-NMR (400 MHz, Chloroform-d) δ 7.59-7.54 (m, 2H), 7.54-7.45 (m, 6H), 7.45-7.39 (m, 2H), 7.37-7.32 (m, 1H), 7.29 (d, J=7.6 Hz, 6H), 7.18 (t, J=7.3 Hz, 3H), 3.25 (d, J=11.7 Hz, 2H), 2.36 (td, J=13.0, 3.2 Hz, 2H), 2.16-2.08 (m, 2H), 1.93 (t, J=12.2 Hz, 2H).

### Step 2: Synthesis of (4-phenyl-1-tritylpiperidin-4-yl)methanamine

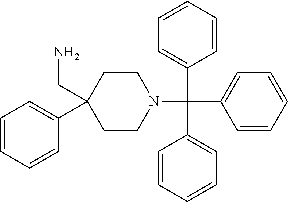 [SMILES: NCC1(c2ccccc2)CCN(C(c2ccccc2)(c2ccccc2)c2ccccc2)CC1]

In round-bottom flask, to a stirred solution of 4-phenyl-1-trityl-piperidine-4-carbonitrile (250 mg, 0.583 mmol) in anhydrous THF (2.5 mL) at 0° C. under nitrogen, was added dropwise 1 M LiAlH₄ (1.5 mL, 1.50 mmol). The reaction mixture was stirred at r.t for 3 h. The reaction mixture was quenched at 0° C. by the dropwise addition of water (57 μL, same weight as LiAlH₄ used), then 1 M NaOH (58 μL, 0.0583 mmol), and subsequently additional water (three times the weight of LiAlH₄, 171 μL). The resulting heterogenous mixture was stirred at room temperature for 30 min, then filtered. The filtrate was dried over Na₂SO₄, filtered, then rinsed with THF and subsequently dichloromethane. The filtrate was concentrated under reduced pressure. The crude was purified by flash chromatography on silica gel using a gradient of methanol (0.7 N ammonia) in dichloromethane from 0.5% to 10%. The desired fractions were combined and concentrated to afford the title compound as a white foam (232.7 mg, 100% purity, 92% yield). ¹H-NMR (400 MHz, Chloroform-d) δ 7.45 (s, 5H), 7.36-7.06 (m, 15H), 3.25-1.25 (2H, br s), 2.67 (s, 2H), 2.31-2.15 (m, 2H), 2.13-1.99 (m, 2H), 1.61-1.28 (m, 4H).

### Step 3: Synthesis of N-((4-phenyl-1-tritylpiperidin-4-yl)methyl)-4-(trifluoromethoxy)benzenesulfonamide

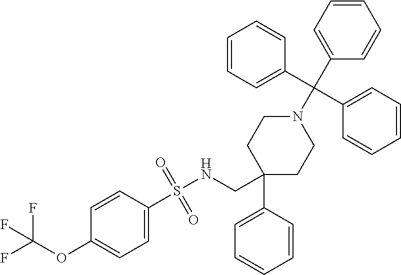 [SMILES: O=S(=O)(NCC1(c2ccccc2)CCN(C(c2ccccc2)(c2ccccc2)c2ccccc2)CC1)c1ccc(OC(F)(F)F)cc1]

In a sealed vial, to a stirred suspension of (4-phenyl-1-trityl-4-piperidyl)methanamine (100 mg, 0.231 mmol) in DCM (1.5 mL) at room temperature were added successively triethylamine (161 μL, 1.16 mmol), 4-(trifluoromethoxy)benzenesulfonyl chloride (98%, 60 μL, 0.348 mmol), and N,N-dimethylpyridin-4-amine (2.9 mg, 0.0238 mmol). The reaction mixture was heated at 40° C. for 1 h. The reaction mixture was cooled to room temperature and diluted with a saturated aqueous solution of NH₄Cl. The layers were separated. The organic layer was washed with a saturated aqueous solution of NaHCO₃, then once with a saturated aqueous solution of NaCl, dried using a phase separator, and concentrated under reduced pressure. The crude material was purified by flash chromatography on silica gel using a gradient of EtOAc in heptane from 5% to 100%. The desired fractions were combined and concentrated to afford the title compound as a colorless gum (134.1 mg, 98% purity, 87% yield). ¹H-NMR (400 MHz, Chloroform-d) δ 7.59 (d, J=7.6 Hz, 2H), 7.50-7.33 (m, 6H), 7.31-7.09 (m, 16H), 3.81 (t, J=6.1 Hz, 1H), 2.91 (d, J=6.3 Hz, 2H), 2.18 (s, 2H), 2.00 (d, J=16.8 Hz, 2H), 1.34-1.21 (m, 2H), 0.92-0.82 (m, 1H).

### Step 4: Synthesis of N-((4-phenylpiperidin-4-yl)methyl)-4-(trifluoromethoxy)benzenesulfonamide (260)

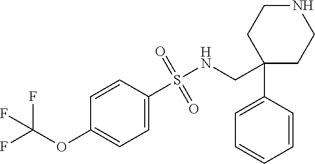 [SMILES: O=S(=O)(NCC1(c2ccccc2)CCNCC1)c1ccc(OC(F)(F)F)cc1]

In a sealed vial, to a stirred solution of N-[(4-phenyl-1-trityl-4-piperidyl)methyl]-4-(trifluoromethoxy)benzenesulfonamide (134 mg, 0.204 mmol) in diethyl ether (2 mL) at room temperature was added 4 M hydrogen chloride in 1,4-dioxane (1.2 mL, 4.80 mmol). The reaction mixture was stirred at room temperature overnight. The resulting suspension was diluted with diethyl ether (15 mL), then filtered, washed with diethyl ether and dried under vacuum at 45° C. for 18 h to afford the HCl salt of the title compound as a white powder (71.5 mg, 100% purity, 78% yield, tᵣ=1.48 min). LCMS (Method C): m/z found 415 [M+H]⁺; ¹H-NMR (400 MHz, Chloroform-d) δ 7.59 (d, J=7.6 Hz, 2H), 7.50-7.33 (m, 6H), 7.31-7.09 (m, 16H), 3.81 (t, J=6.1 Hz, 1H), 2.91 (d, J=6.3 Hz, 2H), 2.18 (s, 2H), 2.00 (d, J=16.8 Hz, 2H), 1.34-1.21 (m, 2H), 0.92-0.82 (m, 1H).

### Example 138: N-(2-(2,5-diazabicyclo[2.2.1]heptan-2-yl)-1-(3,4-dichlorophenyl)ethyl)-4-(trifluoromethoxy)benzenesulfonamide (261a and 261b

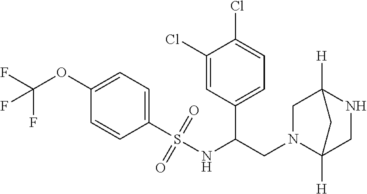 [SMILES: O=S(=O)(NC(CN1CC2CC1CN2)c1ccc(Cl)c(Cl)c1)c1ccc(OC(F)(F)F)cc1]

### Step 1: Synthesis of tert-butyl 5-(2-(3,4-dichlorophenyl)-2-oxoethyl)-2,5-diazabicyclo[2.2.1]heptane-2-carboxylate

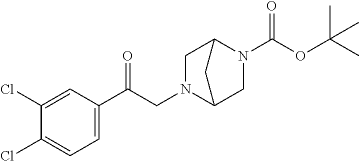 [SMILES: CC(C)(C)OC(=O)N1CC2CC1CN2CC(=O)c1ccc(Cl)c(Cl)c1]

In a sealed tube under nitrogen, to a solution of 2-bromo-1-(3,4-dichlorophenyl)ethanone (97%, 500 mg, 1.81 mmol) and tert-butyl 2,5-diazabicyclo[2.2.2]octane-2-carboxylate (98%, 431 mg, 2.17 mmol) in anhydrous DCM (18 mL) at rt, was added N,N-diethylethanamine (99%, 0.28 mL, 1.99 mmol), then N,N-dimethylpyridin-4-amine (11 mg, 0.0905 mmol), and the solution was stirred at rt for 16 h. To the mixture was added a half saturated aqueous solution of NaHCO₃ and the phases were separated. The aqueous layer was extracted twice with DCM. The combined organic layers were filtered through phase separator and concentrated. The crude material was purified by flash chromatography on silica gel using a gradient of MeOH (0.7 N NH₃) in DCM from 1% to 5% to afford the title compound as a yellow oil (609.4 mg, 93% purity, 81% yield, tᵣ=0.63 min). LCMS (Method F): m/z found 385.3 [M+H]⁺; ¹H-NMR (DMSO-d₆, 400 MHz) δ (ppm) 8.18 (d, J=2.0 Hz, 1H), 7.92 (dd, J=8.4, 2.0 Hz, 1H), 7.80 (d, J=8.4 Hz, 1H), 3.56 (s, 1H), 3.38 (d, J=10.5 Hz, 1H), 3.15-3.05 (m, 1H), 2.94-2.85 (m, 1H), 2.59-2.52 (m, 1H), 1.76 (d, J=9.1 Hz, 1H), 1.68-1.54 (m, 1H), 1.42 (s, 1H), 1.40 (s, 9H), 1.38-1.16 (m, 2H).

### Step 2: Synthesis of tert-butyl 5-(2-amino-2-(3,4-dichlorophenyl)ethyl)-2,5-diazabicyclo[2.2.1]heptane-2-carboxylate

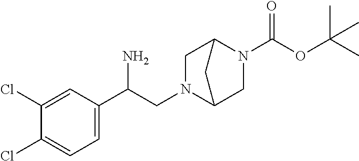 [SMILES: CC(C)(C)OC(=O)N1CC2CC1CN2CC(N)c1ccc(Cl)c(Cl)c1]

In a sealed tube under nitrogen, NaBH₃CN (1.47 g, 2.93 mmol) was added to a stirred solution of tert-butyl 5-[2-(3,4-dichlorophenyl)-2-oxo-ethyl]-2,5-diazabicyclo[2.2.1]heptane-2-carboxylate (93%, 607 mg, 1.47 mmol) and NH₄OAc (98%, 1.15 g, 14.7 mmol) in methanol (9.3 mL). The mixture was stirred at 65° C. for 4 h and filtered. The filtrate was concentrated in vacuo and the residue was diluted in DCM, then a half saturated aqueous solution of NaHCO₃ was added, and the phases were separated. The aqueous layer was extracted twice with DCM and the combined organic phase was washed with brine, dried using a phase separator, and concentrated in vacuo. The crude was purified by flash chromatography on silica gel using a gradient of MeOH (NH₃ 0.7 M) in DCM from 100 to 10%. The desired fractions were combined and concentrated to afford the title compound as a yellow oil (182 mg, 73% purity, 24% yield, tᵣ=0.60 min). LCMS (Method F): m/z found 386.3 [M+H]*; ¹H-NMR (DMSO-d₆, 400 MHz) δ (ppm) 7.64 (dd, J=8.6, 1.9 Hz, 1H), 7.54 (dd, J=8.3, 2.8 Hz, 1H), 7.36 (td, J=8.1, 1.9 Hz, 1H), 4.13 (d, J=11.8 Hz, 1H), 3.89-3.80 (m, 1H), 3.42 (d, J=25.4 Hz, 1H), 3.26 (d, J=9.9 Hz, 1H), 3.03 (d, J=9.1 Hz, 2H), 2.82 (d, J=9.2 Hz, 1H), 2.48-2.39 (m, 2H), 2.01 (s, 2H), 1.74-1.47 (in, 3H).

### Step 3: Synthesis of tert-butyl 5-(2-(3,4-dichlorophenyl)-2-((4-(trifluoromethoxy)phenyl)sulfonamido)ethyl)-2,5-diazabicyclo[2.2.1]heptane-2-carboxylate

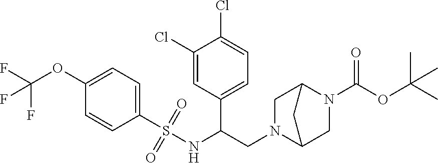 [SMILES: CC(C)(C)OC(=O)N1CC2CC1CN2CC(NS(=O)(=O)c1ccc(OC(F)(F)F)cc1)c1ccc(Cl)c(Cl)c1]

In a sealed tube, 4-(trifluoromethoxy)benzenesulfonyl chloride (98%, 65 μL, 0.375 mmol) was added to a stirred solution of tert-butyl 5-[2-amino-2-(3,4-dichlorophenyl)ethyl]-2,5-diazabicyclo[2.2.1]heptane-2-carboxylate (73%, 180 mg, 0.340 mmol) and triethylamine (100 μL, 0.717 mmol) in DCM (4 mL). The solution was stirred at room temperature overnight. Water and DCM were added, and the aqueous layer was extracted twice with dichloromethane. The combined organic layers were washed with brine, dried using a phase separator, and concentrated under reduced pressure. The crude was purified by flash chromatography on silica gel using a gradient of MeOH (0.7 N NH₃) in DCM from 0.5% to 10%. The desired fractions were combined and concentrated to afford the title compound as two separate diastereomers.

Diastereomer I: (78.4 mg, 98% purity, 38% yield, tᵣ=0.78 min). LCMS (Method F): m/z found 610.3 [M+H]⁺; ¹H-NMR (DMSO-d₆, 600 MHz) δ (ppm) 8.26 (br s, 1H), 7.78 (br dd, J=7.8, 4.8 Hz, 2H), 7.43 (br d, J=7.9 Hz, 4H), 7.21 (br d, J=7.2 Hz, 1H), 4.16-4.25 (m, 1H), 3.99 (br d, J=18.8 Hz, 1H), 3.40 (s, 1H), 3.02-3.15 (m, 1H), 2.94 (br t, J=10.3 Hz, 1H), 2.62-2.70 (m, 1H), 2.51-2.55 (m, 1H), 2.20-2.37 (m, 2H), 1.47-1.57 (m, 2H), 1.32-1.38 (m, 9H).

Diastereomer II: (65.8 mg, 100% purity, 32% yield, tᵣ=0.79 min). LCMS (Method F): m/z found 610.3 [M+H]⁺; ¹H-NMR (DMSO-d₆, 600 MHz): δ (ppm) 8.37 (br s, 1H), 7.75 (br t, J=8.6 Hz, 2H), 7.35-7.40 (m, 4H), 7.16 (br d, J=8.4 Hz, 1H), 4.22-4.28 (m, 1H), 4.06 (br d, J=14.8 Hz, 1H), 3.26-3.29 (m, 1H), 3.16 (br t, J=10.9 Hz, 1H), 2.94-3.04 (m, 1H), 2.68-2.75 (m, 2H), 2.57-2.62 (m, 1H), 2.22-2.28 (m, 1H), 1.46-1.56 (m, 2H), 1.37 (s, 9H).

### Step 4a: Synthesis of N-(2-(2,5-diazabicyclo[2.2.1]heptan-2-yl)-1-(3,4-dichlorophenyl)ethyl)-4-(trifluoromethoxy)benzenesulfonamide (261a

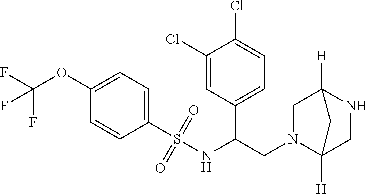 [SMILES: O=S(=O)(NC(CN1CC2CC1CN2)c1ccc(Cl)c(Cl)c1)c1ccc(OC(F)(F)F)cc1]

In a round-bottomed flask, to a stirred suspension of tert-butyl 5-[2-(3,4-dichlorophenyl)-2-[[4-(trifluoromethoxy)phenyl]sulfonylamino]ethyl]-2,5-diazabicyclo[2.2.1]heptane-2-carboxylate (98%, 74 mg, 0.118 mmol, diastereomer I) in diethyl ether (1.1831 mL) at rt under nitrogen was added 2 M hydrogen chloride in Et₂O (0.59 mL, 1.18 mmol). The reaction mixture was stirred at rt for 24 h. The suspension was filtered, washed with Et₂O, and dried overnight at 40° C. to afford a single diastereomer (racemic mixture) of the title compound as a light yellow powder (50 mg, 96.71% purity, 70% yield, tᵣ=1.54 min). LCMS (Method C): m/z found (600 MHz, DMSO-d₆) δ 12.30-10.55 (m, 1H), 10.05-8.26 (m, 2H), 7.88-7.55 (m, 2H), 7.45-7.39 (m, 2H), 7.36 (br dd, J=4.0, 1.6 Hz, 2H), 7.21 (br d, J=8.1 Hz, 1H), 5.24-3.89 (m, 3H), 3.58-2.56 (m, 6H), 2.38 (dt, J=3.7, 1.8 Hz, 2H).

### Step 4b: Synthesis of N-(2-(2,5-diazabicyclo[2.2.1]heptan-2-yl)-1-(3,4-dichlorophenyl)ethyl)-4-(trifluoromethoxy)benzenesulfonamide (261b

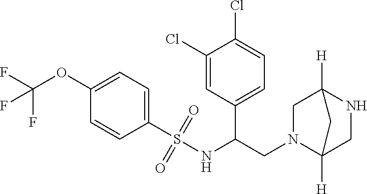 [SMILES: O=S(=O)(NC(CN1CC2CC1CN2)c1ccc(Cl)c(Cl)c1)c1ccc(OC(F)(F)F)cc1]

In a round-bottomed flask, to a stirred suspension of tert-butyl 5-[2-(3,4-dichlorophenyl)-2-[[4-(trifluoromethoxy)phenyl]sulfonylamino]ethyl]-2,5-diazabicyclo[2.2.1]heptane-2-carboxylate (65 mg, 0.102 mmol, diastereomer II) in Et₂O (1.1983 mL) at rt under nitrogen was added 4 M HCl in 1,4-dioxane (250 μL, 0.999 mmol). The reaction mixture was stirred at rt overnight. The suspension was filtered, washed with Et₂O and dried under vacuum. The resulting solid was purified by reverse-phase preparative chromatography using a gradient of acetonitrile in water from 0% to 100% (0.1% CH₃COOH). The desired fractions were combined and concentrated. The resulting residue was solubilized in Et₂O (1 mL) and 2 M hydrogen chloride in Et₂O (204 μL, 0.407 mmol) was added. After 4 h of agitation, the suspension was filtered, washed with Et₂O and dried under vacuum to afford the dihydrochloride salt of the title compound as a white solid (7 mg, 99.83% purity, 11% yield, tᵣ=1.85 min). LCMS (Method C): m/z found 509.9 [M+H]⁺; ¹H-NMR (600 MHz, DMSO-d₆) δ 11.34-10.62 (m, 1H), 10.06-9.31 (m, 1H), 9.19-8.16 (m, 2H), 7.67 (br d, J=8.7 Hz, 2H), 7.37 (br d, J=8.4 Hz, 2H), 7.34 (br d, J=8.1 Hz, 2H), 7.25-7.13 (m, 1H), 5.25-3.98 (m, 3H), 3.56-2.55 (m, 6H), 2.22-1.36 (m, 2H).

### Example 139: 4-(trifluoromethoxy)-N-((1-(3-(trifluoromethyl)phenyl)cyclopentyl)methyl)benzenesulfonamide (262)

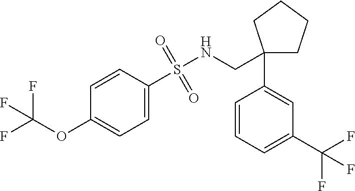 [SMILES: O=S(=O)(NCC1(c2cccc(C(F)(F)F)c2)CCCC1)c1ccc(OC(F)(F)F)cc1]

### Step 1: Synthesis of 1-(3-(trifluoromethyl)phenyl)cyclopentane-1-carbonitrile

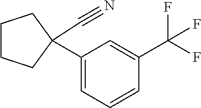 [SMILES: N#CC1(c2cccc(C(F)(F)F)c2)CCCC1]

In a three-neck round-bottomed flask, to a stirred suspension of sodium hydride (60%, 285 mg, 7.13 mmol) in anhydrous DMF (6.644 mL) at 0° C. under nitrogen was added dropwise a solution of [3-(trifluoromethyl)phenyl]acetonitrile (96%, 500 mg, 2.59 mmol) in anhydrous DMF (2.4608 mL). The reaction mixture was stirred at 0° C. for 15 min (until no gas production was observed). A solution of 1,4-dibromobutane (840 mg, 3.89 mmol) in anhydrous DMF (2.4608 mL) was added dropwise at 0° C. and then the reaction mixture was allowed to warm up to room temperature and stirred for 3 h. The reaction mixture was quenched with an half saturated aqueous solution of ammonium chloride (20 mL). Additional half saturated solution (40 mL) was added, followed by ethyl acetate (40 mL). The layers were separated, and the aqueous layer was extracted once more with ethyl acetate (40 mL). The combined organic layers were washed three times with a saturated aqueous solution of sodium chloride (3×25 mL), dried over sodium sulfate, filtered and concentrated under reduced pressure. The crude was purified by flash chromatography on silica gel using a gradient of ethyl acetate in heptane from 2% to 10% to afford the title compound as a colorless oil (532 mg, 86% yield, tᵣ=0.96 min). LCMS (Method F); ¹H-NMR (DMSO-d₆, 400 MHz): δ (ppm) 7.93-7.62 (m, 4H), 2.50-2.41 (m, 2H), 2.19-2.07 (m, 2H), 1.98-1.86 (m, 4H).

### Step 2: Synthesis of (1-(3-(trifluoromethyl)phenyl)cyclopentyl)methanamine

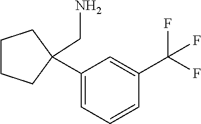 [SMILES: NCC1(c2cccc(C(F)(F)F)c2)CCCC1]

In a round-bottomed flask, to a stirred solution of 1-[3-(trifluoromethyl)phenyl]cyclopentanecarbonitrile (532 mg, 2.22 mmol) in anhydrous THF (13.081 mL) at 0° C. under nitrogen was added 2.4 M LiAlH₄ in THF (2.3 mL, 5.56 mmol). The reaction mixture was stirred at room temperature for 2 h. The reaction mixture was quenched at 0° C. with water (211 μL, same weight as LAH), then 3.75 M sodium hydroxide (211 uL, 0.789 mmol, 15% in water) was added, followed by water (300 wt % LAH). The resulting white suspension was stirred at room temperature overnight, filtered, dried over sodium sulfate, filtered, rinsed with THF, and concentrated under reduced pressure. The crude was purified by flash chromatography on silica gel using a gradient of methanol (0.7N NH₃) in dichloromethane from 0.4% to 10% to afford the title compound as a colorless oil (202 mg, 37% yield, tᵣ=0.6 min). LCMS (Method F): m/z found 244.3 [M+H]⁺; ¹H-NMR (DMSO-d₆, 400 MHz): δ (ppm) 7.67-7.43 (m, 4H), 2.62 (s, 2H), 2.07-1.96 (m, 2H), 1.80-1.55 (m, 6H), 1.05 (s, 2H).

### Step 3: Synthesis of 4-(trifluoromethoxy)-N-((1-(3-(trifluoromethyl)phenyl)cyclopentyl)methyl)benzenesulfonamide (262)

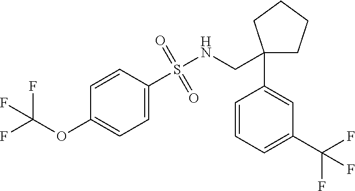 [SMILES: O=S(=O)(NCC1(c2cccc(C(F)(F)F)c2)CCCC1)c1ccc(OC(F)(F)F)cc1]

In a sealed vial, 4-(trifluoromethoxy)benzenesulfonyl chloride (0.070 mL, 0.415 mmol) was added to a stirred solution of [1-[3-(trifluoromethyl)phenyl]cyclopentyl]methanamine (101 mg, 0.415 mmol) and triethylamine (0.23 mL, 1.66 mmol) in DCM (2.7678 mL). The solution was stirred at rt 2.5 h then DCM and an half saturated solution of NaHCO₃ were added and the aqueous layer was extracted twice with DCM. The combined organic layers were filtered through phase separator, then concentrated in vacuo, and purified by flash chromatography on silica gel using a gradient of EtOAc in Heptane from 5% to 25% to afford the title compound as a colorless oil (153.2 mg, 99.8% purity, 79% yield, tᵣ=3.08 min). LCMS (Method C): m/z found 466.2 [M−H]⁻; ¹H-NMR (DMSO-d₆, 500 MHz) δ (ppm) 7.78 (d, J=8.8 Hz, 2H), 7.61-7.43 (m, 7H), 2.95-2.90 (m, 2H), 2.14-1.72 (m, 4H), 1.70-1.52 (m, 4H).

### Example 140: N-((2-phenylpyrrolidin-2-yl)methyl)-4-(trifluoromethoxy)benzenesulfonamide (263)

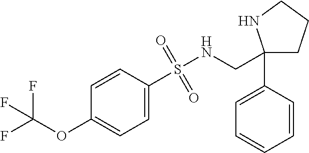 [SMILES: O=S(=O)(NCC1(c2ccccc2)CCCN1)c1ccc(OC(F)(F)F)cc1]

In a vial, to a stirred solution of (2-phenylpyrrolidin-2-yl)methanamine (50 mg, 0.284 mmol) in DCM (2.5 mL) at room temperature were added successively triethylamine (158 μL, 1.13 mmol) and 4-(trifluoromethoxy)benzenesulfonyl chloride (98%, 59 μL, 0.341 mmol). The reaction mixture was stirred at room temperature overnight. The reaction mixture was diluted with dichloromethane and washed with a saturated aqueous solution of NH₄Cl, then a saturated aqueous solution of NaHCO₃ and finally with a saturated aqueous solution of NaCl. The organic layer was dried using a phase separator and concentrated under reduced pressure. The crude was purified by flash chromatography on silica gel using a gradient of methanol (0.7N NH₃) in dichloromethane from 1% to 20%. The desired fractions were combined and concentrated. The residue was converted to the hydrochloride salt by dissolving in diethyl ether (2 mL) with subsequent addition of a solution of 2 M HCl in diethyl ether (1.4 mL, 2.80 mmol). The mixture was stirred for 3 h, filtered, washed with diethyl ether (2×2 mL) and dried under vacuum at 45° C. for 2 days to afford the hydrochloride salt of the title compound as a white powder (67 mg, 99.01% purity, 54% yield, tᵣ=1.14). LCMS (Method H): m/z found 401 [M+H]⁺; ¹H-NMR (600 MHz, DMSO-d₆) δ 9.94 (s, 1H), 9.35 (s, 1H), 8.02 (t, J=7.0 Hz, 1H), 7.88-7.77 (m, 2H), 7.55 (dd, J=8.9, 0.9 Hz, 2H), 7.45-7.32 (m, 5H), 3.44 (s, 1H), 3.25 (qd, J=14.0, 6.9 Hz, 3H), 2.59-2.53 (m, 1H), 2.18-2.07 (m, 2H), 1.99 (dd, J=8.8, 3.6 Hz, 1H).

### Example 141: N-(1-(4-fluorophenyl)-2-(piperazin-1-yl)ethyl)-4-(trifluoromethoxy)benzenesulfonamide (264)

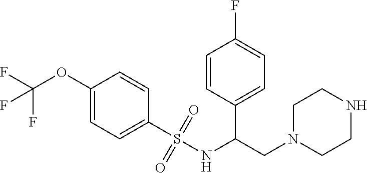 [SMILES: O=S(=O)(NC(CN1CCNCC1)c1ccc(F)cc1)c1ccc(OC(F)(F)F)cc1]

### Step 1: Synthesis of tert-butyl 4-(2-(4-fluorophenyl)-2-oxoethyl)piperazine-1-carboxylate

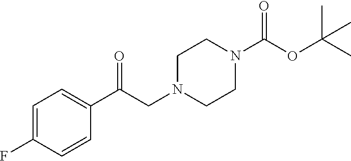 [SMILES: CC(C)(C)OC(=O)N1CCN(CC(=O)c2ccc(F)cc2)CC1]

In a sealed tube, under nitrogen, a solution of 2-bromo-1-(4-fluorophenyl)ethanone (98%, 1.00 g, 4.52 mmol), tert-butyl piperazine-1-carboxylate (1.51 g, 8.13 mmol) and DMAP (28 mg, 0.226 mmol) in DCM (10 mL) was stirred at rt. The mixture was stirred in an iced water bath and triethylamine (692 μL, 4.97 mmol) was added. The cold bath was removed and the mixture was stirred at rt for 16 h. Half saturated aqueous NaHCO₃ was added and the mixture was stirred at rt for 5 min. The aqueous layer was extracted twice with DCM. The combined organic layers were washed with brine, dried over sodium sulfate, filtered and concentrated. The crude was purified by flash chromatography on silica gel using a gradient of (MeOH+2% NH₄OH) in DCM from 0.4% to 4% to afford the title compound as a yellow solid (12.9 g, 94% purity, 91% yield, tᵣ=0.55 min). LCMS (Method E): m/z found 323.2 [M+H]⁺; ¹H-NMR (DMSO-d₆, 400 MHz): δ (ppm) 8.07 (dd, J=8.9, 5.6 Hz, 2H), 7.35 (t, J=8.9 Hz, 2H), 3.87 (s, 2H), 3.32 (d, J=2.6 Hz, 6H), 2.73-2.19 (m, 7H), 1.40 (s, 9H).

### Step 2: Synthesis of tert-butyl 4-(2-amino-2-(4-fluorophenyl)ethyl)piperazine-1-carboxylate

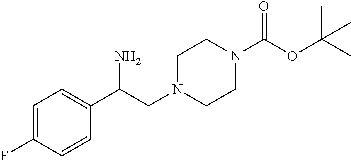 [SMILES: CC(C)(C)OC(=O)N1CCN(CC(N)c2ccc(F)cc2)CC1]

In a sealed tube under nitrogen, polymer bound sodium cyanoborohydride (4.40 g, 8.81 mmol) was added to a stirred solution of tert-butyl 4-[2-(4-fluorophenyl)-2-oxo-ethyl]piperazine-1-carboxylate (1.42 g, 4.40 mmol) and ammonium acetate (3.40 g, 44.0 mmol) in AcOH (2.7 mL) and MeOH (27 mL). The mixture was stirred at 40° C. for 16 h and was filtered. The residue was washed with MeOH and the filtrate was concentrated then dissolved in DCM and saturated aqueous NaHCO₃. The organic layer was washed with saturated aqueous NaHCO₃ then the combined aqueous layers were extracted with DCM. The combined organic layers were washed with brine, dried over sodium sulfate, filtered and concentrated. The crude was purified by flash chromatography on silica gel using a gradient of (MeOH+2% NH₄OH) in DCM from 1% to 5% to afford the title compound as a yellow solid (806 mg, 56% yield, tᵣ=0.57 min). LCMS (Method F): m/z found 324.4 [M+H]⁺; 1H-NMR (DMSO, 400 MHz): δ (ppm) 7.41 (dd, J=8.6, 5.7 Hz, 2H), 7.16-7.06 (m, 2H), 5.76 (s, 1H), 4.05 (dd, J=9.4, 4.5 Hz, 1H), 3.32 (q, J=4.0 Hz, 6H), 2.45 (dt, J=10.7, 5.1 Hz, 2H), 2.38-2.19 (m, 4H), 1.87 (d, J=18.9 Hz, 2H), 1.40 (s, 9H).

### Step 3: Synthesis of tert-butyl 4-(2-(4-fluorophenyl)-2-((4-(trifluoromethoxy)phenyl)sulfonamido)ethyl)piperazine-1-carboxylate

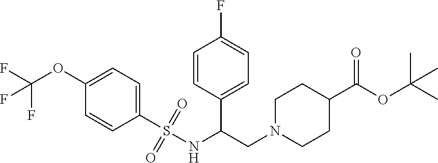 [SMILES: CC(C)(C)OC(=O)C1CCN(CC(NS(=O)(=O)c2ccc(OC(F)(F)F)cc2)c2ccc(F)cc2)CC1]

In a sealed tube under nitrogen, 4-(trifluoromethoxy)benzenesulfonyl chloride (45 μL, 0.267 mmol) was added to a stirred solution of tert-butyl 4-[2-amino-2-(4-fluorophenyl)ethyl]piperazine-1-carboxylate (100 mg, 0.309 mmol) and triethylamine (149 μL, 1.07 mmol) in DCM (2 mL). The solution was stirred at rt for 16 h and was diluted with half saturated aqueous NaHCO₃ and DCM. The aqueous layer was extracted with DCM. The combined organic layers were washed with brine, dried over sodium sulfate, filtered and concentrated. The crude was purified by flash chromatography on silica gel using a gradient of (MeOH+2% NH₄OH) in DCM from 0.2% to 5% to afford the expected compound as a white foam (136 mg, 98% purity, 78% yield, tᵣ=0.74 min). LCMS (Method E): m/z found 548.3 [M+H]⁺; ¹H-NMR (DMSO, 400 MHz): δ (ppm) 8.28 (s, 1H), 7.82-7.65 (m, 2H), 7.47-7.39 (m, 2H), 7.27-7.17 (m, 2H), 7.04-6.89 (m, 2H), 4.39 (dd, J=9.3, 5.1 Hz, 1H), 3.21-2.94 (m, 4H), 2.52 (s, 1H), 2.25 (dq, J=11.5, 5.9 Hz, 5H), 1.37 (s, 9H).

### Step 4: Synthesis of N-(1-(4-fluorophenyl)-2-(piperazin-1-yl)ethyl)-4-(trifluoromethoxy)benzenesulfonamide (264)

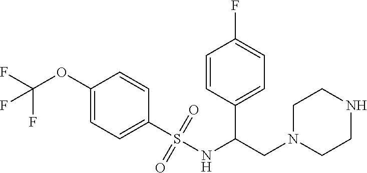 [SMILES: O=S(=O)(NC(CN1CCNCC1)c1ccc(F)cc1)c1ccc(OC(F)(F)F)cc1]

In a sealed tube under nitrogen, 4 M hydrogen chloride in dioxane (1.2 mL, 4.75 mmol) was added to a stirred solution of tert-butyl 4-[2-(4-fluorophenyl)-2-[[4-(trifluoromethoxy)phenyl]sulfonylamino]ethyl]piperazine-1-carboxylate (130 mg, 0.237 mmol) in 1,4-dioxane (1.4 mL). The mixture was stirred at rt for 16 h, then MeOH was added. The mixture was concentrated and MeOH was added to the residue. The suspension was added dropwise to stirred Et₂O and the suspension was stirred at rt for 1 h and filtered. The residue was washed with Et₂O and dried under reduced pressure at 45° C. for 2 days to afford the dihydrochloride salt of the title compound as a white powder (98 mg, 98.4% purity, 78% yield, tᵣ=1.3 min). LCMS (Method H): m/z found 448.5 [M+H]⁺; ¹H-NMR (500 MHz, DMSO-d₆) δ ppm 9.02 (m, 3H), 7.63 (br d, J=8.3 Hz, 2H), 7.31 (br d, J=8.3 Hz, 2H), 7.20 (dd, J=8.1, 5.6 Hz, 2H), 6.89 (t, J=8.8 Hz, 2H), 3.84 (m, 11H).

### Example 142: (S)—N-(2-(4-fluorophenyl)-2-(piperazin-1-yl)ethyl)-4-(trifluoromethoxy)benzenesulfonamide (265)

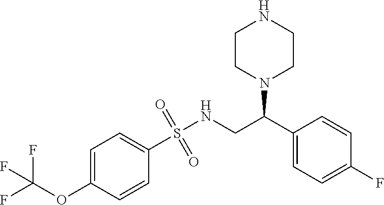 [SMILES: O=S(=O)(NC[C@H](c1ccc(F)cc1)N1CCNCC1)c1ccc(OC(F)(F)F)cc1]

### Step 1: Synthesis of (R)-2-amino-2-(4-fluorophenyl)ethan-1-ol

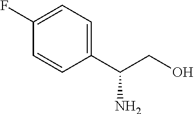 [SMILES: N[C@@H](CO)c1ccc(F)cc1]

In a round-bottom flask, to a stirred suspension of sodium;boranuide (271 mg, 7.17 mmol) in THF (4 mL) at rt under nitrogen was added (R)-4-fluorophenylglycine (97%, 500 mg, 2.87 mmol). The resulting mixture was cooled and stirred at 0° C., then iodine (720 mg, 2.84 mmol) in THF (2 mL) was added dropwise. After decoloration of the solution (from brown to colorless), the white cloudy suspension formed was refluxed overnight. The reaction mixture was cooled to room temperature and MeOH (2 mL) was added dropwise. The mixture was stirred at room temperature and concentrated under reduced pressure. The resulting solid was taken up in potassium hydroxide (20%, 2.0 mL, 7.17 mmol) and stirred at room temperature for 4 h. Dichloromethane was added (15 mL) and the aqueous layer was extracted with dichloromethane (2×10 mL). The combined organic layers were dried over sodium sulfate, filtered and concentrated under reduced pressure to afford the title compound as a white powder (410 mg, 85% purity, 78% yield, tᵣ=0.36 min). LCMS (Method F): m/z found 156.3 [M+H]⁺; ¹H-NMR (DMSO, 400 MHz): δ (ppm) 7.43-7.35 (m, 2H), 7.14-7.07 (m, 2H), 4.75 (t, J=5.5 Hz, 1H), 3.86 (dd, J=7.9, 4.8 Hz, 1H), 3.41 (dt, J=10.3, 5.1 Hz, 1H), 3.27 (ddd, J=10.3, 7.8, 5.3 Hz, 1H), 1.84 (s, 2H).

### Step 2: Synthesis of (R)—N-(1-(4-fluorophenyl)-2-hydroxyethyl)-4-(trifluoromethoxy)benzenesulfonamide

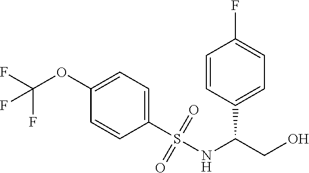 [SMILES: O=S(=O)(N[C@@H](CO)c1ccc(F)cc1)c1ccc(OC(F)(F)F)cc1]

To a stirred solution of (R)-2-amino-2-(4-fluorophenyl)ethan-1-ol (85%, 405 mg, 2.22 mmol) and triethylamine (0.93 mL, 6.66 mmol) in DCM (10.084 mL) at rt under nitrogen was added 4-(trifluoromethoxy)benzenesulfonyl chloride (97%, 0.47 mL, 2.66 mmol). The reaction mixture was stirred at rt overnight. An half saturated solution of NaHCO₃ and DCM were added to the mixture, the layer were separated and the aqueous layer was extracted with DCM. The combined organic layers were dried through phase separator, concentrated in vacuo and purified by flash chromatography on silica gel (column) using a gradient of MeOH (0.7N NH₃) in DCM from 0.4% to 6% to afford the expected compound as a white powder (564 mg, 99% purity, 66% yield, tᵣ=0.83 min). LCMS (Method E); ¹H-NMR (DMSO, 400 MHz): δ (ppm) 8.34 (s, 1H), 7.70-7.64 (m, 2H), 7.34 (dq, J=7.8, 1.2 Hz, 2H), 7.13-7.08 (m, 2H), 6.95-6.84 (m, 2H), 4.88 (t, J=5.7 Hz, 1H), 4.29 (t, J=6.6 Hz, 1H), 3.50 (ddd, J=11.6, 6.9, 5.0 Hz, 1H), 3.41 (dt, J=11.2, 5.9 Hz, 1H).

### Step 3: (R)-2-(4-fluorophenyl)-2-((4-(trifluoromethoxy)phenyl)sulfonamido)ethyl methanesulfonate

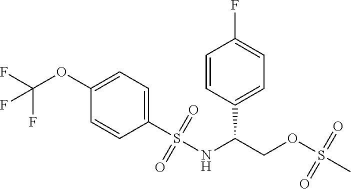 [SMILES: CS(=O)(=O)OC[C@H](NS(=O)(=O)c1ccc(OC(F)(F)F)cc1)c1ccc(F)cc1]

A sealed vial was charged with (R)—N-(1-(4-fluorophenyl)-2-hydroxyethyl)-4-(trifluoromethoxy)benzenesulfonamide (99%, 564 mg, 1.47 mmol) and triethylamine (410 μL, 2.94 mmol) in anhydrous DCM (7.9766 mL). Methanesulfonyl chloride (125 μL, 1.62 mmol) was added and the reaction mixture was stirred at room temperature for 2 h. The reaction mixture was concentrated under reduced pressure and dried under vacuum to afford the expected compound as a pale yellow oil (673 mg, quantitative yield). The product was used in next the step without further purification.

### Step 4: tert-butyl (S)-4-(1-(4-fluorophenyl)-2-((4-(trifluoromethoxy)phenyl)sulfonamido)ethyl)piperazine-1-carboxylate

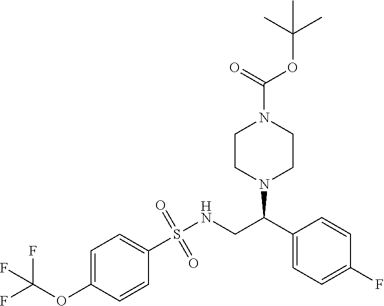 [SMILES: CC(C)(C)OC(=O)N1CCN([C@H](CNS(=O)(=O)c2ccc(OC(F)(F)F)cc2)c2ccc(F)cc2)CC1]

A sealed vial was charged with (R)-2-(4-fluorophenyl)-2-((4-(trifluoromethoxy)phenyl)sulfonamido)ethyl methanesulfonate (673 mg, 1.47 mmol), tert-butyl piperazine-1-carboxylate (97%, 311 mg, 1.62 mmol) and cesium carbonate (576 mg, 1.77 mmol) in anhydrous DMF (6.4 mL). The reaction mixture was stirred at room temperature for 18 h. The reaction mixture was quenched with a half saturated solution of NH₄Cl (100 mL) and EtOAc (50 mL) was added. The layers were separated, and the aqueous layer was extracted with EtOAc (50 mL). The combined organic layers were combined, dried over Na₂SO₄, filtered, concentrated in vacuo and purified by Flash chromatography on silica gel (column) using a gradient of EtOAc in Heptane from 20% to 50% to afford the title compound (538.5 mg, 78% purity, 52% yield, tᵣ=0.78 min) as a mixture further comprising tert-butyl (R)-4-(2-(4-fluorophenyl)-2-((4-(trifluoromethoxy)phenyl)sulfonamido)ethyl)piperazine-1-carboxylate. LCMS (Method E): m/z found [M+H]⁺.

### Step 5: Synthesis of (S)—N-(2-(4-fluorophenyl)-2-(piperazin-1-yl)ethyl)-4-(trifluoromethoxy)benzenesulfonamide

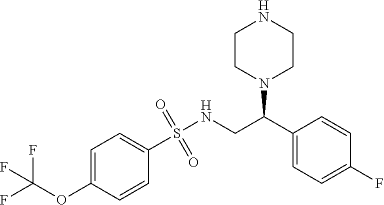 [SMILES: O=S(=O)(NC[C@H](c1ccc(F)cc1)N1CCNCC1)c1ccc(OC(F)(F)F)cc1]

To a stirred mixture of tert-butyl (S)-4-(1-(4-fluorophenyl)-2-((4-(trifluoromethoxy)phenyl)sulfonamido)ethyl)piperazine-1-carboxylate (80%, 427 mg, 0.624 mmol) and tert-butyl (R)-4-(2-(4-fluorophenyl)-2-((4-(trifluoromethoxy)phenyl)sulfonamido)ethyl)piperazine-1-carboxylate (20%, 427 mg, 0.156 mmol) in Et₂O (6.2386 mL) at rt under nitrogen was added 2 M HCl in Et₂O (6.2 mL, 12.5 mmol). The mixture was stirred overnight at rt, filtered and washed with Et₂O. The solid was purified by reverse-phase flash chromatography (column) using a gradient of acetonitrile in water (0.1% AcOH) from 0% to 100% to afford the dihydrochloride salt of the title compound as a white foam (64.1 mg, 100% purity, 20% yield, tᵣ=1.38 min). LCMS (Method H): m/z found 448 [M+H]⁺; ¹H-NMR (500 MHz, DMSO-d₆) δ ppm 2.75-3.79 (m, 10H) 4.36 (br s, 1H) 7.22-7.36 (m, 2H) 7.46-7.65 (m, 4H) 7.81-7.99 (m, 2H).

### Example 143: N-(1-(3,4-dichlorophenyl)-2-(dimethylamino)ethyl)-3-nitro-4-(trifluoromethyl)benzenesulfonamide (266)

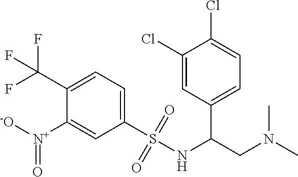

### Step 1: Synthesis of 4-benzylsulfanyl-2-nitro-1-(trifluoromethyl)benzene

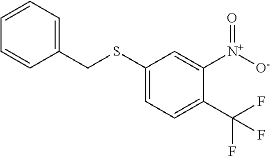 [SMILES: O=[N+]([O-])c1cc(SCc2ccccc2)ccc1C(F)(F)F]

In a sealed tube, a solution of 4-bromo-2-nitro-1-(trifluoromethyl)benzene (500 mg, 1.85 mmol) and DIPEA (647 μL, 3.70 mmol) in 1,4-Dioxane (15 mL) was degassed with argon for 5 min, Tris(dibenzylideneacetone)dipalladium(0) (170 mg, 0.185 mmol), 9,9-Dimethyl-4,5-bis(diphenylphosphino)xanthene (54 mg, 0.0926 mmol) and benzyl mercaptan (460 mg, 3.70 mmol) were added and the mixture was degassed with argon for 5 min. The mixture was stirred at 100° C. for 16 h. The mixture was allowed to cool to rt and was poured into half saturated aqueous solution of NaHCO₃. The aqueous layer was extracted twice with EtOAc. The combined organic layers were washed with brine, dried over sodium sulfate, filtered and concentrated. The crude was purified by flash chromatography on silica gel using a gradient of EtOAc in heptane from 2% to 10% to afford the title compound as a yellow solid (360 mg, 62% yield). ¹H-NMR (DMSO-d₆, 400 MHz): δ (ppm) 8.09 (d, J=1.9 Hz, 1H), 7.89 (d, J=8.4 Hz, 1H), 7.79 (ddd, J=8.4, 2.0, 0.9 Hz, 1H), 7.49-7.42 (m, 2H), 7.39-7.31 (m, 2H), 7.31-7.24 (m, 1H), 4.49 (s, 2H).

### Step 2: Synthesis of N-(1-(3,4-dichlorophenyl)-2-(dimethylamino)ethyl)-3-nitro-4-(trifluoromethyl)benzenesulfonamide (266)

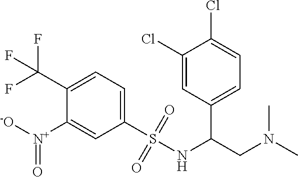

In a sealed tube, a solution of 4-benzylsulfanyl-2-nitro-1-(trifluoromethyl)benzene (90 mg, 0.287 mmol) in acetonitrile (3 mL), water (75 μL) and AcOH (110 μL) was stirred at 0° C. 1,3-dichloro-5,5-dimethyl-imidazolidine-2,4-dione (113 mg, 0.575 mmol) was added and the mixture was stirred at 0° C. for 1 h. triethylamine (44 μL, 0.316 mmol) was added dropwise then a solution of [2-amino-2-(3,4-dichlorophenyl)ethyl]dimethylamine (100 mg, 0.431 mmol) in acetonitrile (150 μL) was added. The mixture was stirred at rt for 64 h and was diluted with half saturated aqueous NaHCO₃. The mixture was extracted three times with DCM, the combined organic layers were washed with brine, dried over sodium sulfate, filtered and concentrated. The crude was purified by flash chromatography on silica gel using a gradient of (MeOH+2% NH₄OH) in DCM from 0.2% to 10%. The desired fractions were concentrated and dissolved in Et₂O. The solution was added dropwise to stirred 2N HCl/Et₂O and the obtained suspension was stirred at rt for 16 h. The suspension was filtered, the residue was washed with Et₂O and dried under reduced pressure at 45° C. for 16 h to afford the hydrochloride salt of the title compound as a white powder (12 mg, 98.4% purity, 7% yield, tᵣ=1.66 min). LCMS (Method C): m/z found 485.9 [M+H]⁺; ¹H-NMR (500 MHz, DMSO-d₆) δ 9.75-9.54 (m, 1H), 9.43-9.15 (m, 1H), 8.16 (s, 1H), 8.07-8.04 (m, 1H), 8.03-7.98 (m, 1H), 7.42 (s, 1H), 7.38 (d, J=8.3 Hz, 1H), 7.23 (br d, J=8.3 Hz, 1H), 5.01-4.83 (m, 1H), 3.53-3.40 (m, 1H), 3.28 (br d, J=3.2 Hz, 1H), 2.98-2.80 (m, 6H).

### Example 144: N-(1-(3,4-dichlorophenyl)-2-(dimethylamino)ethyl)-4-propoxybenzenesulfonamide (267)

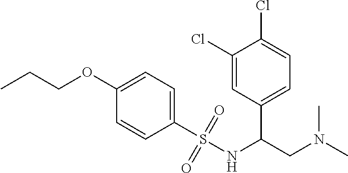 [SMILES: CCCOc1ccc(S(=O)(=O)NC(CN(C)C)c2ccc(Cl)c(Cl)c2)cc1]

In a sealed tube, a solution of 4-propoxybenzenesulfonyl chloride (60 mg, 0.256 mmol) in DCM (500 μL) was added to a stirred solution of [2-amino-2-(3,4-dichlorophenyl)ethyl]dimethylamine (66 mg, 0.281 mmol) and triethylamine (143 μL, 1.02 mmol) in DCM (1 mL). The solution was stirred at rt for 4 h and was diluted with half saturated aqueous solution of NaHCO₃. The aqueous layer was extracted with DCM, the combined organic layers were washed with brine, dried over sodium sulfate, filtered and concentrated. The crude was purified by flash chromatography on silica gel using a gradient of (MeOH+2% NH₄OH) in DCM from 0.2% to 5%. The desired fractions were concentrated and dissolved in Et₂O. The solution was added to stirred HCl 2 N/Et₂O then MeOH was added to dissolve the obtained paste. The solution was stirred at rt for 16 h and was concentrated. Et₂O was added to the residue and the mixture was sonicated. The obtained suspension was filtered, the residue was washed with Et₂O and dried under reduced pressure at 45° C. for 16 h. The solid was dissolved in a minimum of DMSO and was purified by reverse phase chromatography using a gradient of (CH₃CN+0.2% AcOH) in (H₂O+0.2% AcOH) from 0% to 100%. The desired fractions were concentrated and dissolved in Et₂O. The solution was added dropwise to stirred 2 N HCl/Et₂O and the obtained suspension was stirred at rt for 16 h. The suspension was filtered, the residue was washed with Et₂O and dried under reduced pressure at 45° C. for 16 h to afford the hydrochloride salt of the title compound as a white powder (18 mg, 98.3% purity, 14% yield, tᵣ=1.68 min). LCMS (Method C): m/z found 430.9 [M+H]⁺; ¹H-NMR (500 MHz, DMSO-d₆) δ 9.72-9.13 (m, 1H), 0.00 (d, J=10.0 Hz, 1H), 7.41 (d, J=9.0 Hz, 2H), 7.39 (d, J=8.3 Hz, 1H), 7.34 (d, J=2.0 Hz, 1H), 7.21 (dd, J=8.3, 2.0 Hz, 1H), 6.78 (d, J=9.0 Hz, 2H), 4.84-4.69 (m, 1H), 3.96-3.82 (m, J=2.2 Hz, 2H), 3.31-3.16 (m, 2H), 2.99-2.73 (m, 6H), 1.83-1.62 (m, 2H), 0.97 (t, J=7.6 Hz, 3H).

### Example 145: 4-(cyclohexylamino)-N-(1-(3,4-dichlorophenyl)-2-(dimethylamino)ethyl)benzenesulfonamide (268)

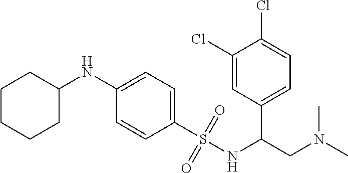 [SMILES: CN(C)CC(NS(=O)(=O)c1ccc(NC2CCCCC2)cc1)c1ccc(Cl)c(Cl)c1]

### Step 1: Synthesis of 4-bromo-N-[1-(3,4-dichlorophenyl)-2-(dimethylamino)ethyl]benzenesulfonamide

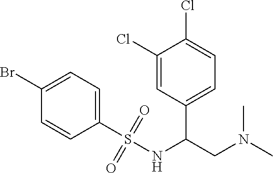 [SMILES: CN(C)CC(NS(=O)(=O)c1ccc(Br)cc1)c1ccc(Cl)c(Cl)c1]

In a round-bottom flask, to a stirred solution of 1-(3,4-dichlorophenyl)-N˜2˜,N˜2˜-dimethylethane-1,2-diamine (90%, 755 mg, 2.91 mmol) in anhydrous DCM (27 mL) at rt under nitrogen were added successively 4-bromobenzenesulfonyl chloride (98%, 760 mg, 2.91 mmol), and triethylamine (1.2 mL, 8.75 mmol). The solution was stirred at rt for 3.5 h and was diluted with water. The aqueous layer was extracted twice with DCM, the combined organic layers were washed with a saturated aqueous solution of NH₄Cl, a saturated aqueous solution of NaHCO₃, and a saturated aqueous solution of NaCl, then dried using a phase separator and concentrated. The crude was purified by flash chromatography on silica gel using a gradient of MeOH in DCM from 0.2% to 5%. The desired fractions were concentrated to afford the title compound as a yellow oil (747.7 mg, 99% purity, 57% yield, tᵣ=1.12 min). LCMS (Method I): m/z found 453.1 [M+H]⁺; ¹H-NMR (400 MHz, DMSO-d₆): δ (ppm) 8.25 (s, 1H), 7.62-7.57 (m, 2H), 7.53-7.47 (m, 2H), 7.41 (d, J=8.3 Hz, 1H), 7.31 (d, J=2.0 Hz, 1H), 7.16 (dd, J=8.3, 2.1 Hz, 1H), 4.34 (t, 1H), 2.42 (dd, J=12.5, 8.2 Hz, 1H), 2.31 (dd, J=12.6, 7.0 Hz, 1H), 2.06 (s, 6H).

### Step 2: Synthesis of 4-(cyclohexylamino)-N-(1-(3,4-dichlorophenyl)-2-(dimethylamino)ethyl)benzenesulfonamide (268)

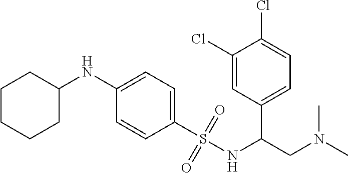 [SMILES: CN(C)CC(NS(=O)(=O)c1ccc(NC2CCCCC2)cc1)c1ccc(Cl)c(Cl)c1]

A sealed vial was charged with 4-bromo-N-[1-(3,4-dichlorophenyl)-2-(dimethylamino)ethyl]benzenesulfonamide (82 mg, 0.181 mmol), cyclohexylamine (41 μL, 0.363 mmol), Cs₂CO₃ (177 mg, 0.544 mmol) and [1-(2-diphenylphosphanyl-1-naphthyl)-2-naphthyl]-diphenyl-phosphane (17 mg, 0.0272 mmol) in anhydrous 1,4-dioxane (3 mL). The reaction mixture was degassed with argon for 15 min. Then Pd(OAc)₂ (4.1 mg, 0.0181 mmol) was added and the reaction mixture was heated at 100° C. overnight. Additional cyclohexylamine (41 μL, 0.363 mmol), [1-(2-diphenylphosphanyl-1-naphthyl)-2-naphthyl]-diphenyl-phosphane (17 mg, 0.0272 mmol), Cs₂CO₃ (177 mg, 0.544 mmol) and Pd(OAc)₂ (4.1 mg, 0.0181 mmol) were added at rt under argon and the reaction mixture was stirred at 100° C. overnight.

The reaction mixture was filtered through talcum powder and the filtrate was washed once with water, once with a saturated aqueous solution of NH₄Cl and once with a saturated aqueous solution of NaCl, dried over sodium sulfate, filtered and concentrated under reduced pressure. The crude was purified by flash chromatography on silica gel using a gradient of MeOH in DCM from 0.2% to 5%. The desired fractions were combined and concentrated. The residue was re-purified by reverse-phase preparative chromatography (Cis-AQ 50 g) using a gradient of acetonitrile in water from 0% to 100% (0.1% AcOH in both solvent). The resulting foam was triturated in pentane, filtered, washed with pentane and dried under vacuum at 70° C. for 20 h to afford the title compound as a yellow solid (10.5 mg, 99.29% purity, 12% yield, tᵣ=1.87 min). LCMS (Method C): m/z found 470 [M+H]⁺; ¹H-NMR (500 MHz, DMSO-d₆) δ 0.00 (d, J=7.1 Hz, 1H), 0.00 (d, J=8.3 Hz, 1H), 0.00 (d, J=2.0 Hz, 1H), 0.00 (d, J=8.8 Hz, 2H), 0.00 (dd, J=8.3, 2.0 Hz, 1H), 0.00 (d, J=8.8 Hz, 2H), 0.00 (d, J=7.8 Hz, 1H), 4.24-4.10 (m, 1H), 3.24-3.12 (m, 1H), 2.37-2.24 (m, 2H), 2.02 (s, 6H), 1.92-1.83 (m, 2H), 1.77-1.67 (m, 2H), 1.64-1.53 (m, 1H), 1.38-1.27 (m, 2H), 1.22-1.07 (m, 3H).

### Example 146: N-(1-(3,4-dichlorophenyl)-2-(4-methylpiperazin-1-yl)ethyl)-4-(4-fluorophenoxy)benzenesulfonamide (269)

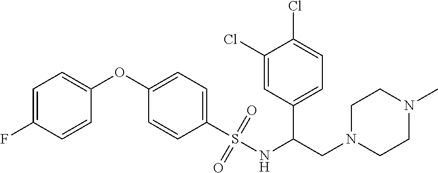 [SMILES: CN1CCN(CC(NS(=O)(=O)c2ccc(Oc3ccc(F)cc3)cc2)c2ccc(Cl)c(Cl)c2)CC1]

### Step 1: Synthesis of 1-(3,4-dichlorophenyl)-2-(4-methylpiperazin-1-yl)ethanone

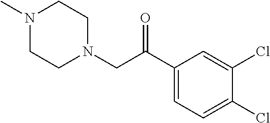 [SMILES: CN1CCN(CC(=O)c2ccc(Cl)c(Cl)c2)CC1]

In a sealed tube under nitrogen, a solution of 2-bromo-1-(3,4-dichlorophenyl)ethanone (97%, 2.00 g, 7.46 mmol) and DMAP (99%, 46 mg, 0.373 mmol) in anhydrous DCM (20 mL) was stirred at rt. Next, 1-methylpiperazine (1.5 mL, 13.4 mmol) then triethylamine (1.1 mL, 8.21 mmol) were added and the solution was stirred at rt for 16 h. The mixture was washed with a half saturated aqueous solution of NaHCO₃. The aqueous layer was extracted twice with DCM. The combined organic layers were washed with brine, dried using a phase separator and concentrated under reduce pressure. The crude was purified by flash chromatography on silica gel using a gradient of MeOH in DCM from 2% to 10% to afford the title compound as a red solid (1,657 g, 92% purity, 71% yield, tᵣ=0.54 min). LCMS (Method F): m/z found 287.2 min [M+H]⁺; ¹H-NMR (400 MHz, DMSO-d₆) δ (ppm): 8.19 (d, J=2.0 Hz, 1H), 7.95 (dd, J=8.4, 2.0 Hz, 1H), 7.80 (d, J=8.4 Hz, 1H), 3.82 (s, 2H), 3.51-3.17 (m, 4H), 2.33 (s, 4H), 2.16 (s, 3H).

### Step 2: Synthesis of 1-(3,4-dichlorophenyl)-2-(4-methylpiperazin-1-yl)ethanone oxime

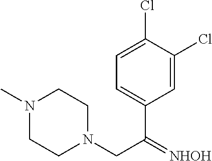

A round-bottom flask was charged with 1-(3,4-dichlorophenyl)-2-(4-methylpiperazin-1-yl)ethanone (92%, 1.66 g, 5.31 mmol), hydroxylamine hydrochloride (738 mg, 10.6 mmol) and DIPEA (1.9 mL, 11.1 mmol) in anhydrous EtOH (30 mL). The flask was sealed and the reaction mixture was stirred at 80° C. overnight. The reaction mixture was concentrated under reduced pressure and dried under vacuum to afford the title compound as an orange solid (3.6 g, 67% purity, quantitative yield, tᵣ=0.56 min). LCMS (Method F): m/z found 302.2 [M+H]⁺; ¹H-NMR (400 MHz, DMSO-d₆) δ (ppm) 11.79 (s, 1H), 7.93 (d, J=2.0 Hz, 1H), 7.72 (dd, J=8.5, 2.0 Hz, 1H), 7.64 (d, J=8.5 Hz, 1H), 3.67 (s, 2H), 2.67 (m, 4H), 2.53 (m, 4H), 2.41 (s, 3H).

### Step 3: Synthesis of 1-(3,4-dichlorophenyl)-2-(4-methylpiperazin-1-yl)ethanamine

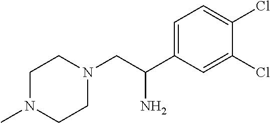 [SMILES: CN1CCN(CC(N)c2ccc(Cl)c(Cl)c2)CC1]

In a round bottom flask under atmospheric pressure, to a stirred solution of 1-(3,4-dichlorophenyl)-2-(4-methylpiperazin-1-yl)ethanone oxime (1.60 g, 5.31 mmol) in anhydrous THF (35 mL) at 0° C. under nitrogen was added 2.4 M LiAlH₄ in THF (7.0 mL, 16.8 mmol). The reaction mixture was stirred at 70° C. for 3 h. The reaction mixture was quenched at 0° C. with water (0.637 mL, same weight as LiAlH₄), then 1 M sodium hydroxide (0.637 mL) was added, followed by additional water (three times the weight as LiAlH₄). The resulting heterogenous mixture was stirred at room temperature for 15 min, dried over sodium sulfate, filtered, rinsed with THF, and concentrated under reduced pressure. The crude was purified by flash chromatography on silica gel using a gradient of (0.7 N NH₃) methanol in dichloromethane from 2% to 8% to afford the title compound as a yellow solid (723 mg, 98% purity, 46% yield, tᵣ=0.43 min). LCMS (Method F): m/z found 288.2 [M+H]⁺; ¹H-NMR (400 MHz, DMSO-d₆) δ (ppm): 7.64 (d, J=2.0 Hz, 1H), 7.54 (d, J=8.3 Hz, 1H), 7.36 (dd, J=8.3, 2.0 Hz, 1H), 4.04 (dd, J=8.8, 5.3 Hz, 1H), 2.47 (s, 2H), 2.40-2.20 (m, 8H), 2.15 (s, 3H), 2.01 (m, 2H).

### Step 4: Synthesis of N-(1-(3,4-dichlorophenyl)-2-(4-methylpiperazin-1-yl)ethyl)-4-(4-fluorophenoxy)benzenesulfonamide (269)

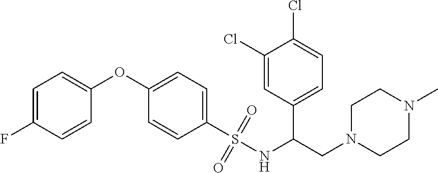 [SMILES: CN1CCN(CC(NS(=O)(=O)c2ccc(Oc3ccc(F)cc3)cc2)c2ccc(Cl)c(Cl)c2)CC1]

In a sealed tube, 4-(4-fluorophenoxy)benzenesulfonyl chloride (95%, 77 mg, 0.255 mmol) was added to a stirred solution of 1-(3,4-dichlorophenyl)-2-(4-methylpiperazin-1-yl)ethanamine (98%, 75 mg, 0.255 mmol) and triethylamine (107 μL, 0.765 mmol) in anhydrous DCM (2.2351 mL). The solution was stirred at room temperature overnight. Water (6 mL) was added and the aqueous layer was extracted with dichloromethane (2×6 mL). The combined organic layers were washed with a saturated aqueous solution of sodium chloride, dried over sodium sulfate, filtered and concentrated under reduced pressure. The crude was purified by flash chromatography on silica gel (column 12 g, 30 μm) using a gradient of methanol in dichloromethane from 0% to 8%. The desired fractions were combined and concentrated. The resulting oil was solubilized in DCM and the homogenous solution was added to pentane. The precipitate formed was filtered, washed with pentane and dried under vacuum at 45° C. for 48 h to afford the title compound as a white powder (77.1 mg, 98.34% purity, 55.216% yield, tᵣ=1.72 min). LCMS (Method C): m/z found 538.3 [M+H]⁺; ¹H-NMR (400 MHz, DMSO-d₆, 300 K) 6 ppm 8.04 (br d, J=7.2 Hz, 1H), 7.55-7.65 (m, 2H), 7.45 (d, J=8.3 Hz, 1H), 7.38 (d, J=1.9 Hz, 1H), 7.27-7.35 (m, 2H), 7.18 (dd, J=8.3, 2.1 Hz, 1H), 7.06-7.15 (m, 2H), 6.85-6.96 (m, 2H), 4.35 (q, J=7.3 Hz, 1H), 2.11-2.49 (m, 10H), 2.10 (s, 3H).

### Example 147: Cell Viability Assay

### LNCaP Cell Line

LNCaP cell line was used for the cell viability assay. LNCaP cells were plated in 96-well plates at a density of 5000 cells per well. After 24 hours of plating, the cells were treated with increasing doses of compound (e.g., compounds 1-269) ranging from 1 μM to 80 μM. Relative cell numbers were analyzed after 48 hours using 3-(4,5-dimethylthiazol-2-yl)-5-(3-carboxymethoxyphenyl)-2-(4-sulfophenyl)-2H-tetrazolium (MTS) assay (Promega) according to the manufacturer's instructions.

### HCC1937 Cell Line

HCC1937 cells are plated in a 96 well plate format at a density of 10,000 cells per well. After incubation overnight, cells are treated with a compound (e.g., compound 263) at following concentrations: 80, 40, 20, 10, 5, 2.5, 1.25 μM. Compound concentrations are prepared in RPMI via serial dilutions from a starting stock of 80 mM. 48 hours after compound treatment, cell viability is assessed using the Promega CellTiter 96 aqueous one proliferation assay (Promega G3582, MTS assay) according to manufacturer's protocol.

The cell viability data was analyzed with GraphPad Prism software. The values were log transformed and analyzed with non-linear regression (curve-fit) using log(inhibitor) vs. response-variable slope (four parameters) and constraining the bottom to equal zero. The results are provided in Table 113. Unless otherwise indicated, the GI₅₀ values provided in Table 113 correspond to the LNCaP cell viability assays.

TABLE 113
Cell viability assay data for compounds 1-269

| No. | Structure | Nomenclature | GI₅₀ (μM) |
|---|---|---|---|
| 1 | 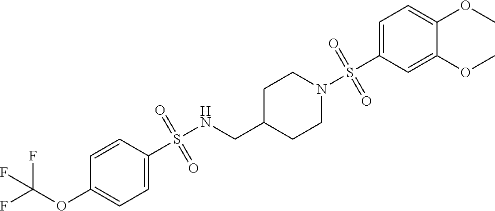 [SMILES: COc1ccc(S(=O)(=O)N2CCC(CNS(=O)(=O)c3ccc(OC(F)(F)F)cc3)CC2)cc1OC] | N-((1-((3,4- dimethoxyphenyl)sulfonyl)piperidin-4-yl) methyl)-4-(trifluoromethoxy) benzenesulfonamide | 63.75 |
| 2 | 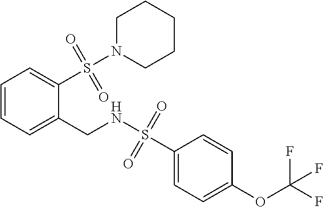 [SMILES: O=S(=O)(NCc1ccccc1S(=O)(=O)N1CCCCC1)c1ccc(OC(F)(F)F)cc1] | N-(2-(piperidin-1- ylsulfonyl)benzyl)-4- (trifluoromethoxy)benzenesulfonamide | 58.3 |
| 3 | 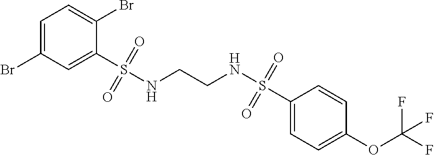 [SMILES: O=S(=O)(NCCNS(=O)(=O)c1cc(Br)ccc1Br)c1ccc(OC(F)(F)F)cc1] | 2,5-dibromo-N-(2-((4- (trifluoromethoxy)phenyl)sulfonamido) ethyl)benzenesulfonamide | 24.6 |
| 4 | 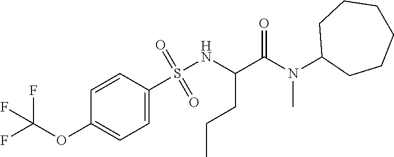 [SMILES: CCCC(NS(=O)(=O)c1ccc(OC(F)(F)F)cc1)C(=O)N(C)C1CCCCCC1] | N-cycloheptyl-N-methyl-2-((4- (trifluoromethoxy)phenyl)sulfonamido) pentanamide | 53.96 |
| 5 | 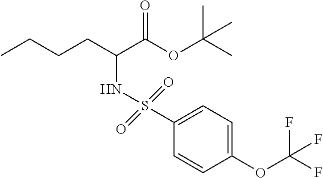 [SMILES: CCCCC(NS(=O)(=O)c1ccc(OC(F)(F)F)cc1)C(=O)OC(C)(C)C] | tert-butyl 2-((4- (trifluoromethoxy)phenyl)sulfonamido) hexanoate | 39.49 |
| 6 | 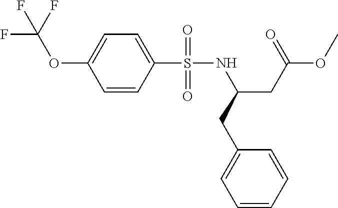 [SMILES: COC(=O)C[C@@H](Cc1ccccc1)NS(=O)(=O)c1ccc(OC(F)(F)F)cc1] | methyl (R)-4-phenyl-3-((4- (trifluoromethoxy)phenyl) sulfonamido)butanoate | 61.08 |
| 7 | 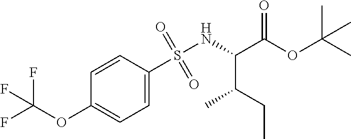 [SMILES: CC[C@H](C)[C@H](NS(=O)(=O)c1ccc(OC(F)(F)F)cc1)C(=O)OC(C)(C)C] | tert-butyl ((4- (trifluoromethoxy)phenyl)sulfonyl)-L- isoleucinate | 57.91 |
| 8 | 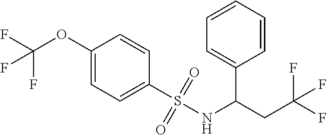 [SMILES: O=S(=O)(NC(CC(F)(F)F)c1ccccc1)c1ccc(OC(F)(F)F)cc1] | N-(3,3,3-trifluoro-1- phenylpropyl)-4-(trifluoromethoxy) benzenesulfonamide | 38.09 |
| 9 | 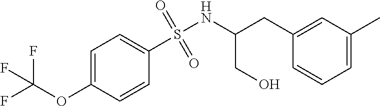 [SMILES: Cc1cccc(CC(CO)NS(=O)(=O)c2ccc(OC(F)(F)F)cc2)c1] | N-(1-hydroxy-3-(m-tolyl)propan- 2-yl)-4- (trifluoromethoxy)benzenesulfonamide | 24.05 |
| 10 | 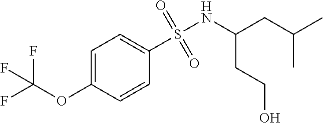 [SMILES: CC(C)CC(CCO)NS(=O)(=O)c1ccc(OC(F)(F)F)cc1] | N-(1-hydroxy-5-methylhexan-3- yl)-4- (trifluoromethoxy)benzenesulfonamide | 5.47 |
| 11 | 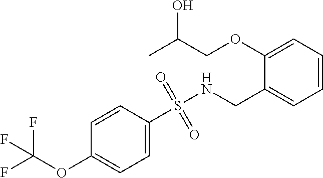 [SMILES: CC(O)COc1ccccc1CNS(=O)(=O)c1ccc(OC(F)(F)F)cc1] | N-(2-(2-hydroxypropoxy)benzyl)- 4- (trifluoromethoxy)benzenesulfonamide | 30.89 |
| 12 | 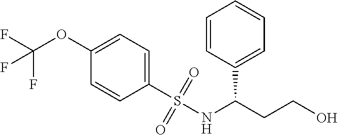 [SMILES: O=S(=O)(N[C@@H](CCO)c1ccccc1)c1ccc(OC(F)(F)F)cc1] | (S)-N-(3-hydroxy-1- phenylpropyl)-4- (trifluoromethoxy)benzenesulfonamide | 34.73 |
| 13 | 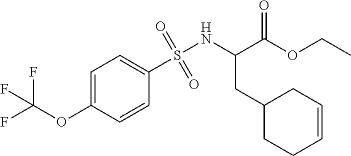 [SMILES: CCOC(=O)C(CC1CC=CCC1)NS(=O)(=O)c1ccc(OC(F)(F)F)cc1] | ethyl 3-(cyclohex-3-en-1-yl)-2-((4- (trifluoromethoxy)phenyl)sulfonamido) propanoate | 53.46 |
| 14 | 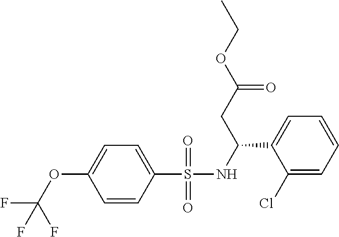 [SMILES: CCOC(=O)C[C@@H](NS(=O)(=O)c1ccc(OC(F)(F)F)cc1)c1ccccc1Cl] | ethyl (R)-3-(2-chlorophenyl)-3- ((4- (trifluoromethoxy)phenyl) sulfonamido)propanoate | 51.88 |
| 15 | 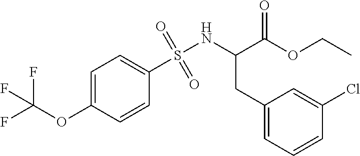 [SMILES: CCOC(=O)C(Cc1cccc(Cl)c1)NS(=O)(=O)c1ccc(OC(F)(F)F)cc1] | ethyl 3-(3-chlorophenyl)-2-((4- (trifluoromethoxy)phenyl) sulfonamido)propanoate | 49.55 |
| 16 | 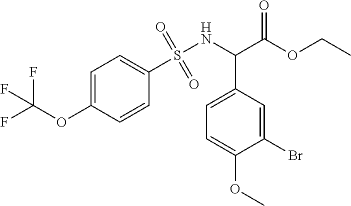 [SMILES: CCOC(=O)C(NS(=O)(=O)c1ccc(OC(F)(F)F)cc1)c1ccc(OC)c(Br)c1] | ethyl 2-(3-bromo-4- methoxyphenyl)-2-((4- (trifluoromethoxy)phenyl)sulfonamido) acetate | 49.72 |
| 17 | 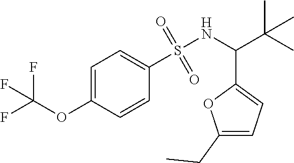 [SMILES: CCc1ccc(C(NS(=O)(=O)c2ccc(OC(F)(F)F)cc2)C(C)(C)C)o1] | N-(1-(5-ethylfuran-2-yl)-2,2- dimethylpropyl)-4- (trifluoromethoxy)benzenesulfonamide | 55.56 |
| 18 | 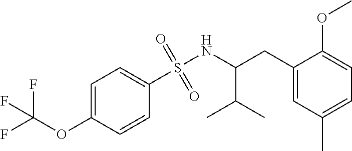 [SMILES: COc1ccc(C)cc1CC(NS(=O)(=O)c1ccc(OC(F)(F)F)cc1)C(C)C] | N-(1-(2-methoxy-5- methylphenyl)-3-methylbutan-2-yl)-4- (trifluoromethoxy)benzenesulfonamide | 76.67 |
| 19 | 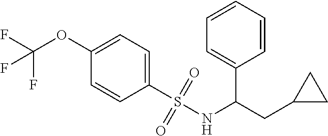 [SMILES: O=S(=O)(NC(CC1CC1)c1ccccc1)c1ccc(OC(F)(F)F)cc1] | N-(2-cyclopropyl-1-phenylethyl)- 4- (trifluoromethoxy)benzenesulfonamide | 51.88 |
| 20 | 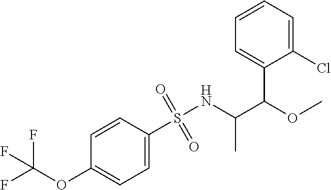 [SMILES: COC(c1ccccc1Cl)C(C)NS(=O)(=O)c1ccc(OC(F)(F)F)cc1] | N-(1-(2-chlorophenyl)-1- methoxypropan-2-yl)-4- (trifluoromethoxy)benzenesulfonamide | 50.93 |
| 21 | 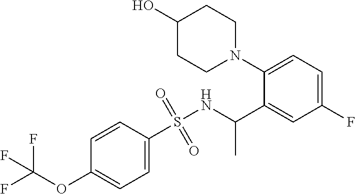 [SMILES: CC(NS(=O)(=O)c1ccc(OC(F)(F)F)cc1)c1cc(F)ccc1N1CCC(O)CC1] | N-(1-(5-fluoro-2-(4- hydroxypiperidin-1- yl)phenyl)ethyl)-4- (trifluoromethoxy)benzenesulfonamide | 46.8 |
| 22 | 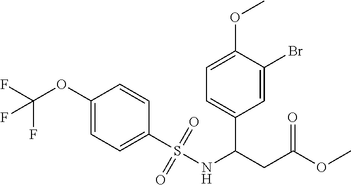 [SMILES: COC(=O)CC(NS(=O)(=O)c1ccc(OC(F)(F)F)cc1)c1ccc(OC)c(Br)c1] | methyl 3-(3-bromo-4- methoxyphenyl)-3-((4- (trifluoromethoxy)phenyl)sulfonamido) propanoate | 39.09 |
| 23 | 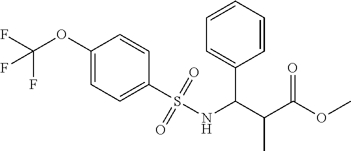 [SMILES: COC(=O)C(C)C(NS(=O)(=O)c1ccc(OC(F)(F)F)cc1)c1ccccc1] | methyl 2-methyl-3-phenyl-3-((4- (trifluoromethoxy)phenyl)sulfonamido) propanoate | 43.15 |
| 24 | 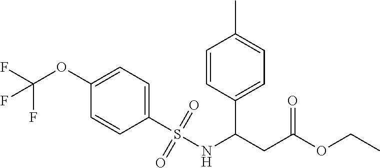 [SMILES: CCOC(=O)CC(NS(=O)(=O)c1ccc(OC(F)(F)F)cc1)c1ccc(C)cc1] | ethyl 3-(p-tolyl)-3-((4- (trifluoromethoxy)phenyl)sulfonamido) propanoate | 44.22 |
| 25 | 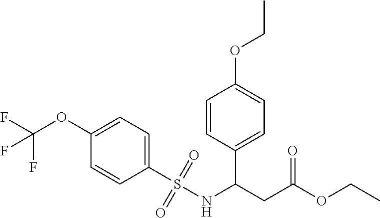 [SMILES: CCOC(=O)CC(NS(=O)(=O)c1ccc(OC(F)(F)F)cc1)c1ccc(OCC)cc1] | ethyl 3-(4-ethoxyphenyl)-3-((4- (trifluoromethoxy)phenyl)sulfonamido) propanoate | 75.57 |
| 26 | 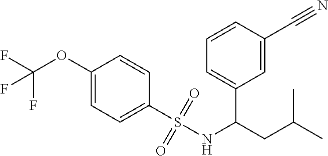 [SMILES: CC(C)CC(NS(=O)(=O)c1ccc(OC(F)(F)F)cc1)c1cccc(C#N)c1] | N-(1-(3-cyanophenyl)-3- methylbutyl)-4- (trifluoromethoxy)benzenesulfonamide | 28.85 |
| 27 | 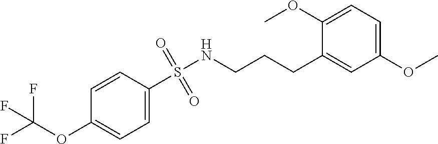 [SMILES: COc1ccc(OC)c(CCCNS(=O)(=O)c2ccc(OC(F)(F)F)cc2)c1] | N-(3-(2,5- dimethoxyphenyl)propyl)-4- (trifluoromethoxy)benzenesulfonamide | 40.24 |
| 28 | 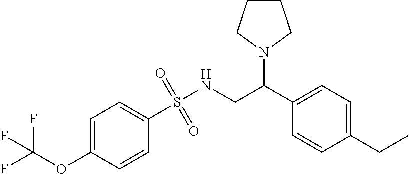 [SMILES: CCc1ccc(C(CNS(=O)(=O)c2ccc(OC(F)(F)F)cc2)N2CCCC2)cc1] | N-(2-(4-ethylphenyl)-2- (pyrrolidin-1-yl)ethyl)-4- (trifluoromethoxy)benzenesulfonamide | 55.81 |
| 29 | 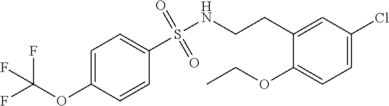 [SMILES: CCOc1ccc(Cl)cc1CCNS(=O)(=O)c1ccc(OC(F)(F)F)cc1] | N-(5-chloro-2-ethoxyphenethyl)-4- (trifluoromethoxy)benzenesulfonamide | 38.32 |
| 30 | 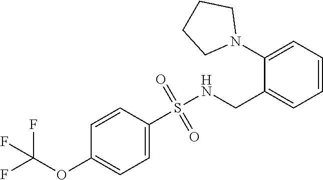 [SMILES: O=S(=O)(NCc1ccccc1N1CCCC1)c1ccc(OC(F)(F)F)cc1] | N-(2-(pyrrolidin-1-yl)benzyl)-4- (trifluoromethoxy)benzenesulfonamide | 49.67 |
| 31 | 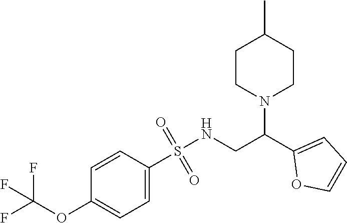 [SMILES: CC1CCN(C(CNS(=O)(=O)c2ccc(OC(F)(F)F)cc2)c2ccco2)CC1] | N-(2-(furan-2-yl)-2-(4- methylpiperidin-1-yl)ethyl)-4- (trifluoromethoxy)benzenesulfonamide | 45.9 |
| 32 | 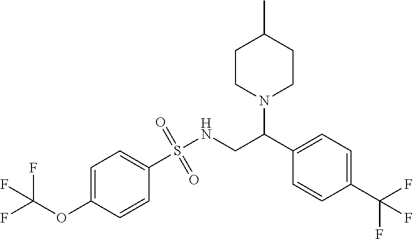 [SMILES: CC1CCN(C(CNS(=O)(=O)c2ccc(OC(F)(F)F)cc2)c2ccc(C(F)(F)F)cc2)CC1] | N-(2-morpholino-2-(4- (trifluoromethyl)phenyl)ethyl)-4- (trifluoromethoxy)benzenesulfonamide | 45.01 |
| 33 | 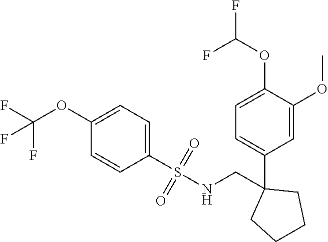 [SMILES: COc1cc(C2(CNS(=O)(=O)c3ccc(OC(F)(F)F)cc3)CCCC2)ccc1OC(F)F] | N-((1-(4-(difluoromethoxy)-3- methoxyphenyl)cyclopentyl)methyl)-4- (trifluoromethoxy)benzenesulfonamide | 73.4 |
| 34 | 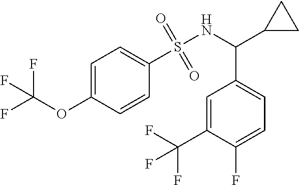 [SMILES: O=S(=O)(NC(c1ccc(F)c(C(F)(F)F)c1)C1CC1)c1ccc(OC(F)(F)F)cc1] | N-(cyclopropyl(4-fluoro-3- (trifluoromethyl)phenyl)methyl)-4- (trifluoromethoxy)benzenesulfonamide | 40.1 |
| 35 | 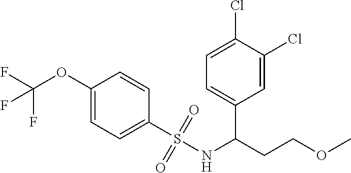 [SMILES: COCCC(NS(=O)(=O)c1ccc(OC(F)(F)F)cc1)c1ccc(Cl)c(Cl)c1] | N-(1-(3,4-dichlorophenyl)-3- methoxypropyl)-4- (trifluoromethoxy)benzenesulfonamide | 50.92 |
| 36 | 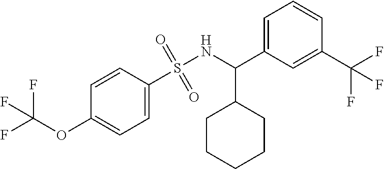 [SMILES: O=S(=O)(NC(c1cccc(C(F)(F)F)c1)C1CCCCC1)c1ccc(OC(F)(F)F)cc1] | N-(cyclohexyl(3- (trifluoromethyl)phenyl)methyl)-4- (trifluoromethoxy)benzenesulfonamide | 29.09 |
| 37 | 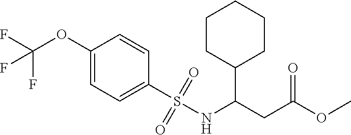 [SMILES: COC(=O)CC(NS(=O)(=O)c1ccc(OC(F)(F)F)cc1)C1CCCCC1] | methyl 3-cyclohexyl-3-((4- (trifluoromethoxy)phenyl) sulfonamido)propanoate | 60.03 |
| 38 | 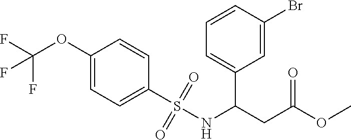 [SMILES: COC(=O)CC(NS(=O)(=O)c1ccc(OC(F)(F)F)cc1)c1cccc(Br)c1] | methyl 3-(3-bromophenyl)-3-((4- (trifluoromethoxy)phenyl)sulfonamido) propanoate | 66.51 |
| 39 | 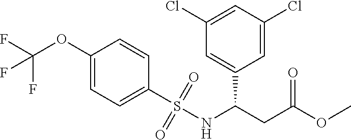 [SMILES: COC(=O)C[C@H](NS(=O)(=O)c1ccc(OC(F)(F)F)cc1)c1cc(Cl)cc(Cl)c1] | methyl (S)-3-(3,5-dichlorophenyl)- 3-((4- (trifluoromethoxy)phenyl)sulfonamido) propanoate | 35.27 |
| 40 | 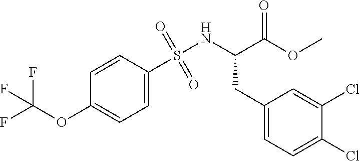 [SMILES: COC(=O)[C@H](Cc1ccc(Cl)c(Cl)c1)NS(=O)(=O)c1ccc(OC(F)(F)F)cc1] | methyl (S)-3-(3,4-dichlorophenyl)- 2-((4- (trifluoromethoxy)phenyl)sulfonamido) propanoate | 57.2 |
| 41 | 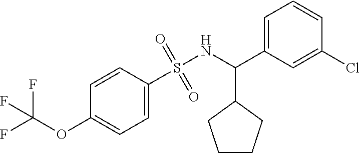 [SMILES: O=S(=O)(NC(c1cccc(Cl)c1)C1CCCC1)c1ccc(OC(F)(F)F)cc1] | N-((3- chlorophenyl)(cyclopentyl)methyl)-4- (trifluoromethoxy)benzenesulfonamide | 37.29 |
| 42 | 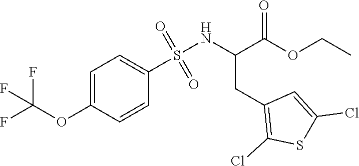 [SMILES: CCOC(=O)C(Cc1cc(Cl)sc1Cl)NS(=O)(=O)c1ccc(OC(F)(F)F)cc1] | ethyl 3-(2,5-dichlorothiophen-3- yl)-2-((4- (trifluoromethoxy)phenyl)sulfonamido) propanoate | 57.8 |
| 43 | 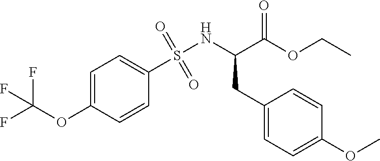 [SMILES: CCOC(=O)[C@@H](Cc1ccc(OC)cc1)NS(=O)(=O)c1ccc(OC(F)(F)F)cc1] | ethyl (R)-3-(4-methoxyphenyl)-2- ((4- (trifluoromethoxy)phenyl)sulfonamido) propanoate | 72.0 |
| 44 | 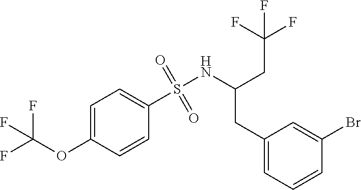 [SMILES: O=S(=O)(NC(Cc1cccc(Br)c1)CC(F)(F)F)c1ccc(OC(F)(F)F)cc1] | N-(1-(3-bromophenyl)-4,4,4- trifluorobutan-2-yl)-4- (trifluoromethoxy)benzenesulfonamide | 59 |
| 45 | 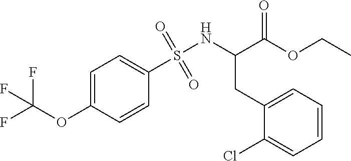 [SMILES: CCOC(=O)C(Cc1ccccc1Cl)NS(=O)(=O)c1ccc(OC(F)(F)F)cc1] | ethyl 3-(2-chlorophenyl)-2-((4- (trifluoromethoxy)phenyl)sulfonamido) propanoate | 46.0 |
| 46 | 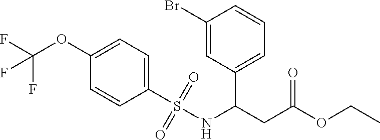 [SMILES: CCOC(=O)CC(NS(=O)(=O)c1ccc(OC(F)(F)F)cc1)c1cccc(Br)c1] | ethyl 3-(3-bromophenyl)-3-((4- (trifluoromethoxy)phenyl)sulfonamido) propanoate | 49.0 |
| 47 | 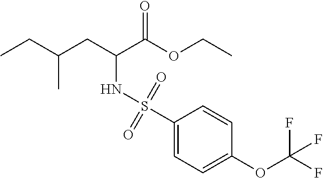 [SMILES: CCOC(=O)C(CC(C)CC)NS(=O)(=O)c1ccc(OC(F)(F)F)cc1] | ethyl 4-methyl-2-((4- (trifluoromethoxy)phenyl)sulfonamido) hexanoate | 70.0 |
| 48 | 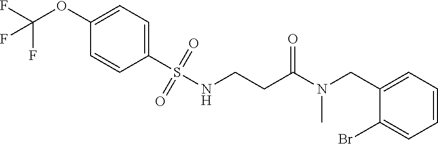 [SMILES: CN(Cc1ccccc1Br)C(=O)CCNS(=O)(=O)c1ccc(OC(F)(F)F)cc1] | N-(2-bromobenzyl)-N-methyl-3- ((4- (trifluoromethoxy)phenyl)sulfonamido) propanamide | 43.0 |
| 49 | 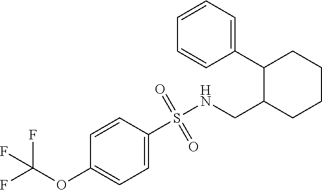 [SMILES: O=S(=O)(NCC1CCCCC1c1ccccc1)c1ccc(OC(F)(F)F)cc1] | N-((2-phenylcyclohexyl)methyl)- 4- (trifluoromethoxy)benzenesulfonamide | 39.0 |
| 50 | 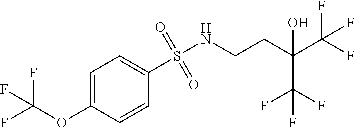 [SMILES: O=S(=O)(NCCC(O)(C(F)(F)F)C(F)(F)F)c1ccc(OC(F)(F)F)cc1] | N-(4,4,4-trifluoro-3-hydroxy-3- (trifluoromethyl)butyl)-4- (trifluoromethoxy)benzenesulfonamide | 71.0 |
| 51 | 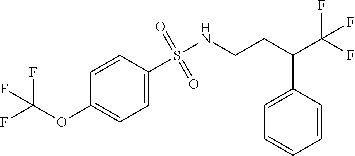 [SMILES: O=S(=O)(NCCC(c1ccccc1)C(F)(F)F)c1ccc(OC(F)(F)F)cc1] | N-(4,4,4-trifluoro-3-phenylbutyl)- 4- (trifluoromethoxy)benzenesulfonamide | 55.00 |
| 52 | 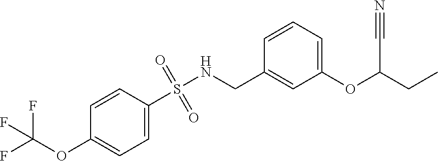 [SMILES: CCC(C#N)Oc1cccc(CNS(=O)(=O)c2ccc(OC(F)(F)F)cc2)c1] | N-(3-(1-cyanopropoxy)benzyl)-4- (trifluoromethoxy)benzenesulfonamide | 72.0 |
| 53 | 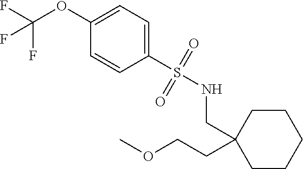 [SMILES: COCCC1(CNS(=O)(=O)c2ccc(OC(F)(F)F)cc2)CCCCC1] | N-((1-(2- methoxyethyl)cyclohexyl)methyl)- 4- (trifluoromethoxy)benzenesulfonamide | 66.0 |
| 54 | 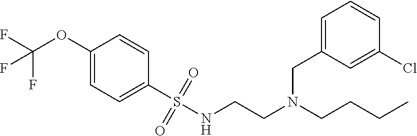 [SMILES: CCCCN(CCNS(=O)(=O)c1ccc(OC(F)(F)F)cc1)Cc1cccc(Cl)c1] | N-(2-(butyl(3- chlorobenzyl)amino)ethyl)-4- (trifluoromethoxy)benzenesulfonamide | 33.0 |
| 55 | 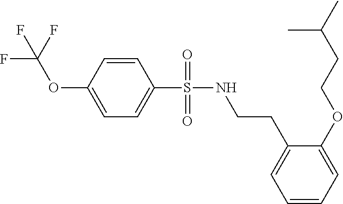 [SMILES: CC(C)CCOc1ccccc1CCNS(=O)(=O)c1ccc(OC(F)(F)F)cc1] | N-(2-(isopentyloxy)phenethyl)-4- (trifluoromethoxy)benzenesulfonamide | 57.0 |
| 56 | 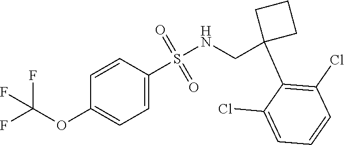 [SMILES: O=S(=O)(NCC1(c2c(Cl)cccc2Cl)CCC1)c1ccc(OC(F)(F)F)cc1] | N-((1-(2,6- dichlorophenyl)cyclobutyl)methyl)-4- (trifluoromethoxy)benzenesulfonamide | 30.0 |
| 57 | 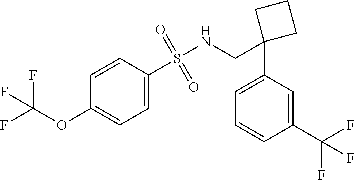 [SMILES: O=S(=O)(NCC1(c2cccc(C(F)(F)F)c2)CCC1)c1ccc(OC(F)(F)F)cc1] | 4-(trifluoromethoxy)-N-((1-(3- (trifluoromethyl)phenyl)cyclobutyl) methyl)benzenesulfonamide | 38.0 |
| 58 | 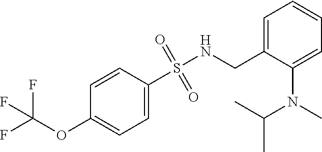 [SMILES: CC(C)N(C)c1ccccc1CNS(=O)(=O)c1ccc(OC(F)(F)F)cc1] | N-(2- (isopropyl(methyl)amino)benzyl)- 4- (trifluoromethoxy)benzenesulfonamide | 20.0 |
| 59 | 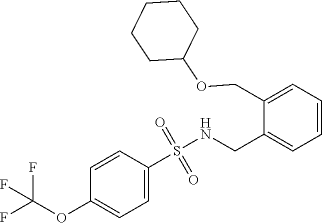 [SMILES: O=S(=O)(NCc1ccccc1COC1CCCCC1)c1ccc(OC(F)(F)F)cc1] | N-(2- ((cyclohexyloxy)methyl)benzyl)-4- (trifluoromethoxy)benzenesulfonamide | 23.0 |
| 60 | 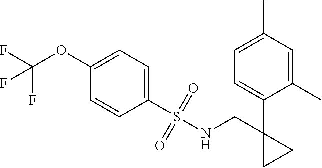 [SMILES: Cc1ccc(C2(CNS(=O)(=O)c3ccc(OC(F)(F)F)cc3)CC2)c(C)c1] | N-((1-(2,4- dimethylphenyl)cyclopropyl)methyl)-4- (trifluoromethoxy)benzenesulfonamide | 44.0 |
| 61 | 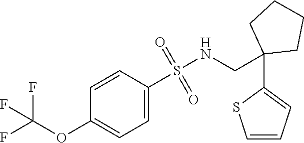 [SMILES: O=S(=O)(NCC1(c2cccs2)CCCC1)c1ccc(OC(F)(F)F)cc1] | N-((1-(thiophen-2- yl)cyclopentyl)methyl)-4- (trifluoromethoxy)benzenesulfonamide | 38 |
| 62 | 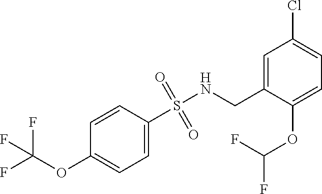 [SMILES: O=S(=O)(NCc1cc(Cl)ccc1OC(F)F)c1ccc(OC(F)(F)F)cc1] | N-(5-chloro-2- (difluoromethoxy)benzyl)-4- (trifluoromethoxy)benzenesulfonamide | 70.0 |
| 63 | 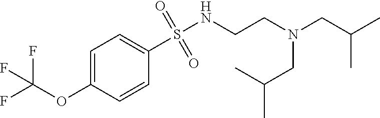 [SMILES: CC(C)CN(CCNS(=O)(=O)c1ccc(OC(F)(F)F)cc1)CC(C)C] | N-(2-(diisobutylamino)ethyl)-4- (trifluoromethoxy)benzenesulfonamide | 60.0 |
| 64 | 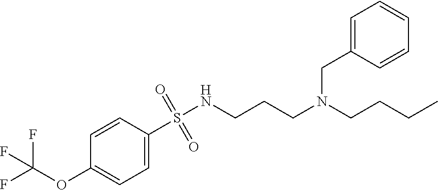 [SMILES: CCCCN(CCCNS(=O)(=O)c1ccc(OC(F)(F)F)cc1)Cc1ccccc1] | N-(3-(benzyl(butyl)amino)propyl)- 4-(trifluoromethoxy)benzenesulfonamide | 36.0 |
| 65 | 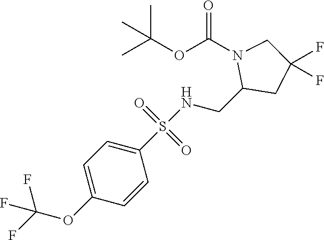 [SMILES: CC(C)(C)OC(=O)N1CC(F)(F)CC1CNS(=O)(=O)c1ccc(OC(F)(F)F)cc1] | tert-butyl 4,4-difluoro-2-(((4- (trifluoromethoxy)phenyl)sulfonamido) methyl)pyrrolidine-1-carboxylate | 43.0 |
| 66 | 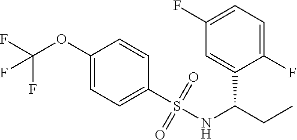 [SMILES: CC[C@H](NS(=O)(=O)c1ccc(OC(F)(F)F)cc1)c1cc(F)ccc1F] | (S)-N-(1-(2,5- difluorophenyl)propyl)-4- (trifluoromethoxy)benzenesulfonamide | 35.0 |
| 67 | 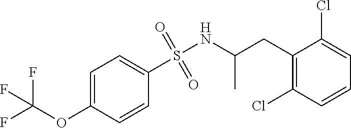 [SMILES: CC(Cc1c(Cl)cccc1Cl)NS(=O)(=O)c1ccc(OC(F)(F)F)cc1] | N-(1-(2,6-dichlorophenyl)propan- 2-yl)-4- (trifluoromethoxy)benzenesulfonamide | 50 |
| 68 | 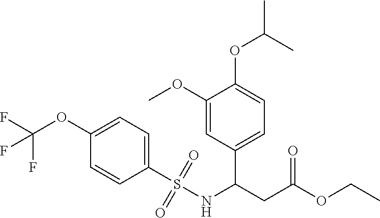 [SMILES: CCOC(=O)CC(NS(=O)(=O)c1ccc(OC(F)(F)F)cc1)c1ccc(OC(C)C)c(OC)c1] | ethyl 3-(4-isopropoxy-3- methoxyphenyl)-3-((4- (trifluoromethoxy)phenyl)sulfonamido) propanoate | 72.0 |
| 69 | 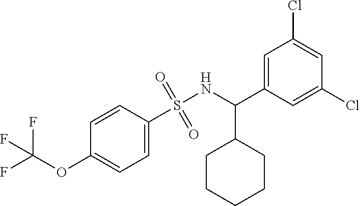 [SMILES: O=S(=O)(NC(c1cc(Cl)cc(Cl)c1)C1CCCCC1)c1ccc(OC(F)(F)F)cc1] | N-(cyclohexyl(3,5- dichlorophenyl)methyl)-4- (trifluoromethoxy)benzenesulfonamide | 9.0 |
| 70 | 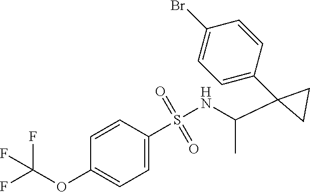 [SMILES: CC(NS(=O)(=O)c1ccc(OC(F)(F)F)cc1)C1(c2ccc(Br)cc2)CC1] | N-(1-(1-(4- bromophenyl)cyclopropyl)ethyl)-4- (trifluoromethoxy)benzenesulfonamide | 25 |
| 71 | 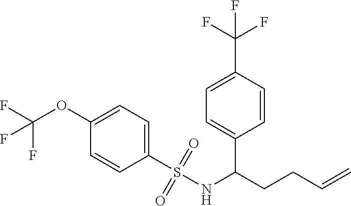 [SMILES: C=CCCC(NS(=O)(=O)c1ccc(OC(F)(F)F)cc1)c1ccc(C(F)(F)F)cc1] | 4-(trifluoromethoxy)-N-(1-(4- (trifluoromethyl)phenyl)pent-4-en- 1-yl)benzenesulfonamide | 20.0 |
| 72 | 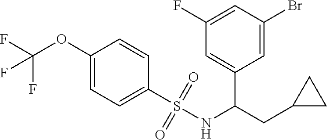 [SMILES: O=S(=O)(NC(CC1CC1)c1cc(F)cc(Br)c1)c1ccc(OC(F)(F)F)cc1] | N-(1-(3-bromo-5-fluorophenyl)-2- cyclopropylethyl)-4- (trifluoromethoxy)benzenesulfonamide | 17.0 |
| 73 | 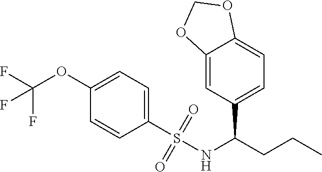 [SMILES: CCC[C@@H](NS(=O)(=O)c1ccc(OC(F)(F)F)cc1)c1ccc2c(c1)OCO2] | (R)-N-(1-(benzo[d][1,3]dioxol-5- yl)butyl)-4- (trifluoromethoxy)benzenesulfonamide | 38.0 |
| 74 | 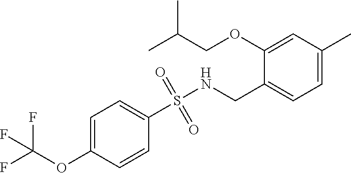 [SMILES: Cc1ccc(CNS(=O)(=O)c2ccc(OC(F)(F)F)cc2)c(OCC(C)C)c1] | N-(2-isobutoxy-4-methylbenzyl)- 4-(trifluoromethoxy)benzenesulfonamide | 60.0 |
| 75 | 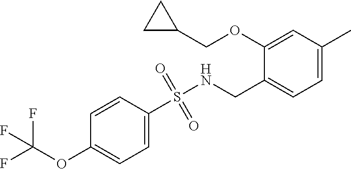 [SMILES: Cc1ccc(CNS(=O)(=O)c2ccc(OC(F)(F)F)cc2)c(OCC2CC2)c1] | N-(2-(cyclopropylmethoxy)-4- methylbenzyl)-4- (trifluoromethoxy)benzenesulfonamide | 30.0 |
| 76 | 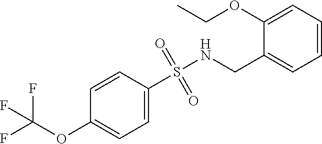 [SMILES: CCOc1ccccc1CNS(=O)(=O)c1ccc(OC(F)(F)F)cc1] | N-(2-ethoxybenzyl)-4- (trifluoromethoxy)benzenesulfonamide | 60.0 |
| 77 | 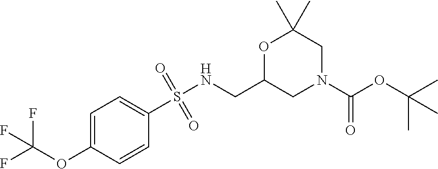 [SMILES: CC(C)(C)OC(=O)N1CC(CNS(=O)(=O)c2ccc(OC(F)(F)F)cc2)OC(C)(C)C1] | tert-butyl 2,2-dimethyl-6-(((4- (trifluoromethoxy)phenyl)sulfonamido) methyl)morpholine-4-carboxylate | 20.0 |
| 78 | 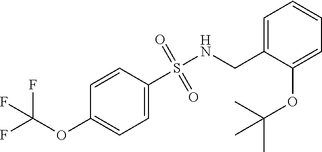 [SMILES: CC(C)(C)Oc1ccccc1CNS(=O)(=O)c1ccc(OC(F)(F)F)cc1] | N-(2-(tert-butoxy)benzyl)-4- (trifluoromethoxy)benzenesulfonamide | 46.0 |
| 79 | 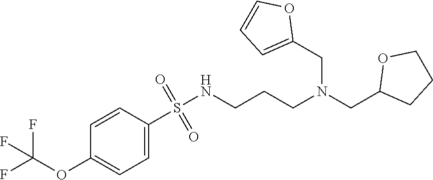 [SMILES: O=S(=O)(NCCCN(Cc1ccco1)CC1CCCO1)c1ccc(OC(F)(F)F)cc1] | N-(3-((furan-2- ylmethyl)((tetrahydrofuran-2- yl)methyl)amino)propyl)-4- (trifluoromethoxy)benzenesulfonamide | 49.0 |
| 80 | 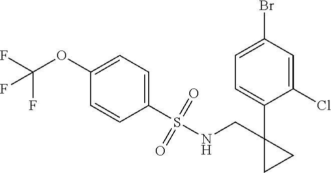 [SMILES: O=S(=O)(NCC1(c2ccc(Br)cc2Cl)CC1)c1ccc(OC(F)(F)F)cc1] | N-((1-(4-bromo-2- chlorophenyl)cyclopropyl)methyl)-4- (trifluoromethoxy)benzenesulfonamide | 23.0 |
| 81 | 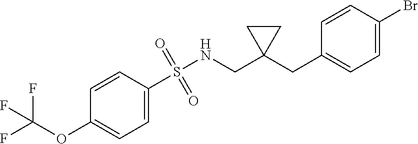 [SMILES: O=S(=O)(NCC1(Cc2ccc(Br)cc2)CC1)c1ccc(OC(F)(F)F)cc1] | N-((1-(4- bromobenzyl)cyclopropyl)methyl)-4- (trifluoromethoxy)benzenesulfonamide | 40.0 |
| 82 | 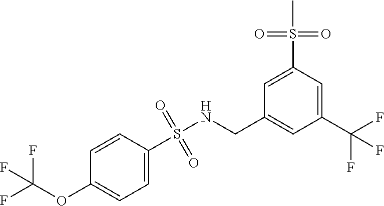 [SMILES: CS(=O)(=O)c1cc(CNS(=O)(=O)c2ccc(OC(F)(F)F)cc2)cc(C(F)(F)F)c1] | N-(3-(methylsulfonyl)-5- (trifluoromethyl)benzyl)-4- (trifluoromethoxy)benzenesulfonamide | 64.0 |
| 83 | 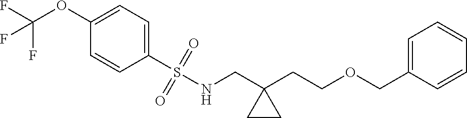 [SMILES: O=S(=O)(NCC1(CCOCc2ccccc2)CC1)c1ccc(OC(F)(F)F)cc1] | N-((1-(2- (benzyloxy)ethyl)cyclopropyl)methyl)-4- (trifluoromethoxy)benzenesulfonamide | 38.0 |
| 84 | 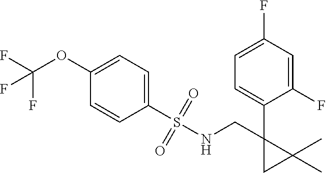 [SMILES: CC1(C)CC1(CNS(=O)(=O)c1ccc(OC(F)(F)F)cc1)c1ccc(F)cc1F] | N-((1-(2,4-difluorophenyl)-2,2- dimethylcyclopropyl)methyl)-4- (trifluoromethoxy)benzenesulfonamide | 18.0 |
| 85 | 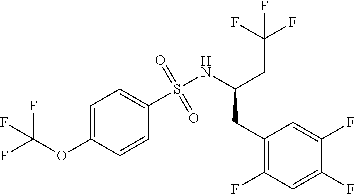 [SMILES: O=S(=O)(N[C@H](Cc1cc(F)c(F)cc1F)CC(F)(F)F)c1ccc(OC(F)(F)F)cc1] | (R)-N-(4,4,4-trifluoro-1-(2,4,5- trifluorophenyl)butan-2-yl)-4- (trifluoromethoxy)benzenesulfonamide | 26 |
| 86 | 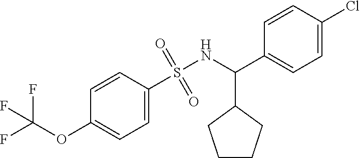 [SMILES: O=S(=O)(NC(c1ccc(Cl)cc1)C1CCCC1)c1ccc(OC(F)(F)F)cc1] | N-((4- chlorophenyl)(cyclopentyl)methyl)-4- (trifluoromethoxy)benzenesulfonamide | 75.0 |
| 87 | 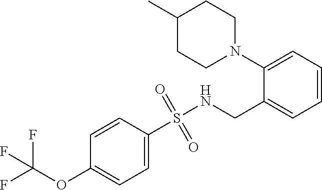 [SMILES: CC1CCN(c2ccccc2CNS(=O)(=O)c2ccc(OC(F)(F)F)cc2)CC1] | N-(2-(4-methylpiperidin-1- yl)benzyl)-4- (trifluoromethoxy)benzenesulfonamide | 23.0 |
| 88 | 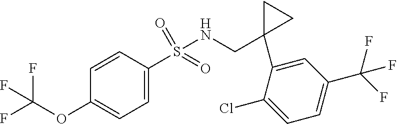 [SMILES: O=S(=O)(NCC1(c2cc(C(F)(F)F)ccc2Cl)CC1)c1ccc(OC(F)(F)F)cc1] | N-((1-(2-chloro-5- (trifluoromethyl)phenyl)cyclopropyl) methyl)-4- (trifluoromethoxy)benzenesulfonamide | 25 |
| 89 | 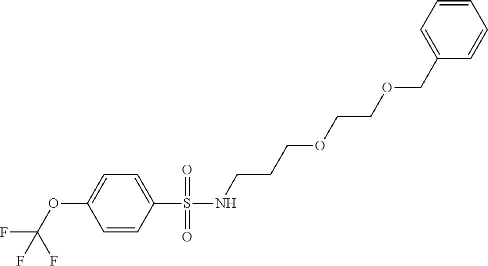 [SMILES: O=S(=O)(NCCCOCCOCc1ccccc1)c1ccc(OC(F)(F)F)cc1] | N-(3-(2- (benzyloxy)ethoxy)propyl)-4- (trifluoromethoxy)benzenesulfonamide | 48.0 |
| 90 | 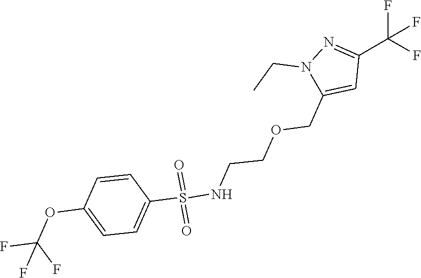 [SMILES: CCn1nc(C(F)(F)F)cc1COCCNS(=O)(=O)c1ccc(OC(F)(F)F)cc1] | N-(2-((1-ethyl-3-(trifluoromethyl)- 1H-pyrazol-5-yl)methoxy)ethyl)- 4- (trifluoromethoxy)benzenesulfonamide | 28 |
| 91 | 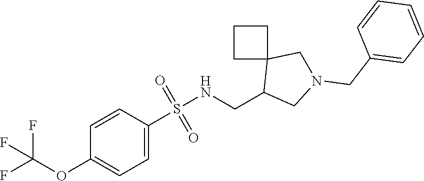 [SMILES: O=S(=O)(NCC1CN(Cc2ccccc2)CC12CCC2)c1ccc(OC(F)(F)F)cc1] | N-((6-benzyl-6-azaspiro[3.4]octan- 8-yl)methyl)-4- (trifluoromethoxy)benzenesulfonamide | 28 |
| 92 | 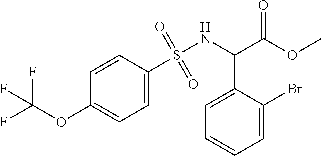 [SMILES: COC(=O)C(NS(=O)(=O)c1ccc(OC(F)(F)F)cc1)c1ccccc1Br] | methyl 2-(2-bromophenyl)-2-((4- (trifluoromethoxy)phenyl) sulfonamido)acetate | 42.0 |
| 93 | 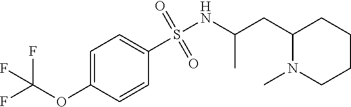 [SMILES: CC(CC1CCCCN1C)NS(=O)(=O)c1ccc(OC(F)(F)F)cc1] | N-(1-(1-methylpiperidin-2-yl)propan-2- yl)-4-(trifluoromethoxy) benzenesulfonamide | 34.0 |
| 94 | 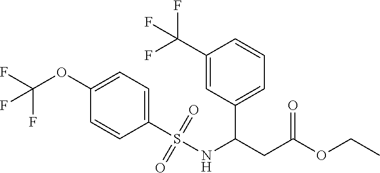 [SMILES: CCOC(=O)CC(NS(=O)(=O)c1ccc(OC(F)(F)F)cc1)c1cccc(C(F)(F)F)c1] | ethyl 3-((4- (trifluoromethoxy)phenyl)sulfonamido)- 3-(3- (trifluoromethyl)phenyl)propanoate | 25.0 |
| 95 | 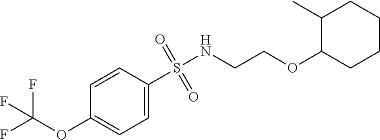 [SMILES: CC1CCCCC1OCCNS(=O)(=O)c1ccc(OC(F)(F)F)cc1] | N-(2-((2- methylcyclohexyl)oxy)ethyl)-4- (trifluoromethoxy)benzenesulfonamide | 15.0 |
| 96 | 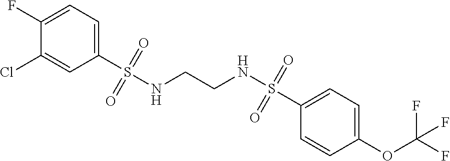 [SMILES: O=S(=O)(NCCNS(=O)(=O)c1ccc(F)c(Cl)c1)c1ccc(OC(F)(F)F)cc1] | 3-chloro-4-fluoro-N-(2-((4- (trifluoromethoxy)phenyl) sulfonamido)ethyl)benzenesulfonamide | 31.0 |
| 97 | 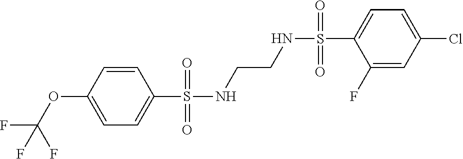 [SMILES: O=S(=O)(NCCNS(=O)(=O)c1ccc(Cl)cc1F)c1ccc(OC(F)(F)F)cc1] | 4-chloro-2-fluoro-N-(2-((4- (trifluoromethoxy)phenyl) sulfonamido)ethyl)benzenesulfonamide | 41.0 |
| 98 | 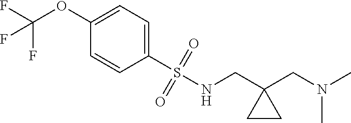 [SMILES: CN(C)CC1(CNS(=O)(=O)c2ccc(OC(F)(F)F)cc2)CC1] | N-((1- ((dimethylamino)methyl)cyclopropyl) methyl)-4- (trifluoromethoxy)benzenesulfonamide | 29.0 |
| 99 | 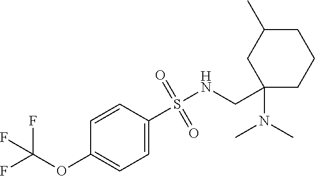 [SMILES: CC1CCCC(CNS(=O)(=O)c2ccc(OC(F)(F)F)cc2)(N(C)C)C1] | N-((1-(dimethylamino)-3- methylcyclohexyl)methyl)-4- (trifluoromethoxy)benzenesulfonamide | 45 |
| 100 | 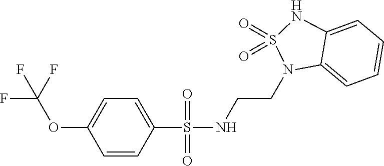 [SMILES: O=S(=O)(NCCN1c2ccccc2NS1(=O)=O)c1ccc(OC(F)(F)F)cc1] | N-(2-(2,2- dioxidobenzo[c][1,2,5]thiadiazol- 1(3H)-yl)ethyl)-4- (trifluoromethoxy)benzenesulfonamide | 61.0 |
| 101 | 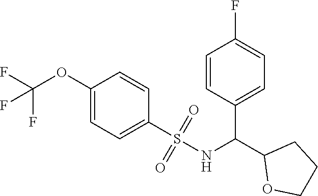 [SMILES: O=S(=O)(NC(c1ccc(F)cc1)C1CCCO1)c1ccc(OC(F)(F)F)cc1] | N-((4- fluorophenyl)(tetrahydrofuran-2- yl)methyl)-4- (trifluoromethoxy)benzenesulfonamide | 33.0 |
| 102 | 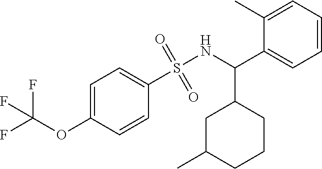 [SMILES: Cc1ccccc1C(NS(=O)(=O)c1ccc(OC(F)(F)F)cc1)C1CCCC(C)C1] | N-((3-methylcyclohexyl)(o- tolyl)methyl)-4- (trifluoromethoxy)benzenesulfonamide | 14.0 |
| 103 | 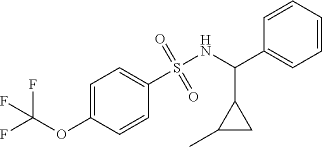 [SMILES: CC1CC1C(NS(=O)(=O)c1ccc(OC(F)(F)F)cc1)c1ccccc1] | N-((2- methylcyclopropyl)(phenyl)methyl)-4- (trifluoromethoxy)benzenesulfonamide | 20.0 |
| 104 | 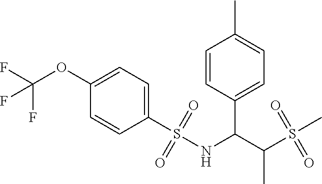 [SMILES: Cc1ccc(C(NS(=O)(=O)c2ccc(OC(F)(F)F)cc2)C(C)S(C)(=O)=O)cc1] | N-(2-(methylsulfonyl)-1-(p- tolyl)propyl)-4- (trifluoromethoxy)benzenesulfonamide | 49.0 |
| 105 | 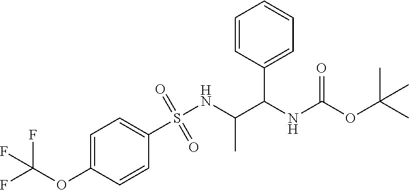 [SMILES: CC(NS(=O)(=O)c1ccc(OC(F)(F)F)cc1)C(NC(=O)OC(C)(C)C)c1ccccc1] | tert-butyl (1-phenyl-2-((4- (trifluoromethoxy)phenyl)sulfonamido) propyl)carbamate | 37.0 |
| 106 | 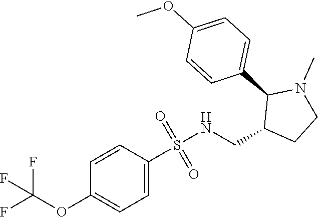 [SMILES: COc1ccc([C@@H]2[C@@H](CNS(=O)(=O)c3ccc(OC(F)(F)F)cc3)CCN2C)cc1] | rac-N-(((2S,3R)-2-(4- methoxyphenyl)-1- methylpyrrolidin-3-yl)methyl)-4- (trifluoromethoxy)benzenesulfonamide | 24.0 |
| 107 | 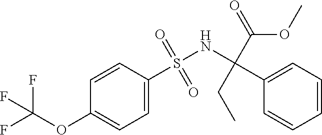 [SMILES: CCC(NS(=O)(=O)c1ccc(OC(F)(F)F)cc1)(C(=O)OC)c1ccccc1] | methyl 2-phenyl-2-((4- (trifluoromethoxy)phenyl)sulfonamido) butanoate | 69.0 |
| 108 | 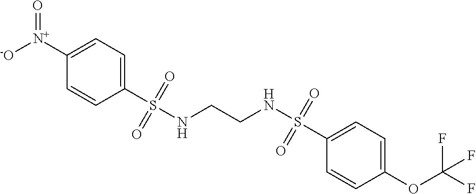 | 4-nitro-N-(2-((4- (trifluoromethoxy)phenyl)sulfonamido) ethyl)benzenesulfonamide | 65.0 |
| 109 | 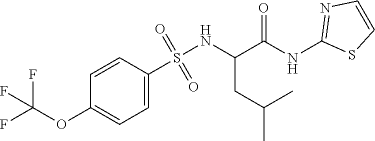 [SMILES: CC(C)CC(NS(=O)(=O)c1ccc(OC(F)(F)F)cc1)C(=O)Nc1nccs1] | 4-methyl-N-(thiazol-2-yl)-2-((4- (trifluoromethoxy)phenyl)sulfonamido) pentanamide | 25 |
| 110 | 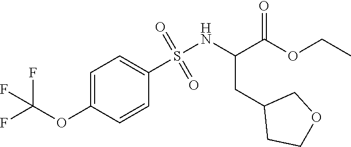 [SMILES: CCOC(=O)C(CC1CCOC1)NS(=O)(=O)c1ccc(OC(F)(F)F)cc1] | ethyl 3-(tetrahydrofuran-3-yl)-2- ((4- (trifluoromethoxy)phenyl) sulfonamido)propanoate | 61.0 |
| 111 | 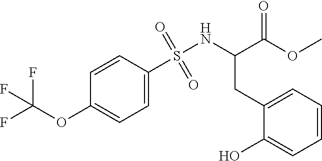 [SMILES: COC(=O)C(Cc1ccccc1O)NS(=O)(=O)c1ccc(OC(F)(F)F)cc1] | methyl 3-(2-hydroxyphenyl)-2-((4- (trifluoromethoxy)phenyl)sulfonamido) propanoate | 50 |
| 112 | 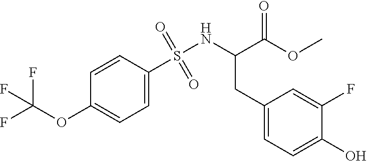 [SMILES: COC(=O)C(Cc1ccc(O)c(F)c1)NS(=O)(=O)c1ccc(OC(F)(F)F)cc1] | methyl 3-(3-fluoro-4- hydroxyphenyl)-2-((4- (trifluoromethoxy)phenyl)sulfonamido) propanoate | 38.0 |
| 113 | 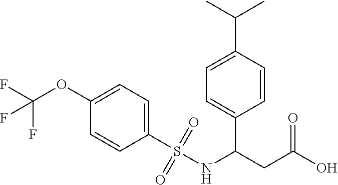 [SMILES: CC(C)c1ccc(C(CC(=O)O)NS(=O)(=O)c2ccc(OC(F)(F)F)cc2)cc1] | 3-(4-isopropylphenyl)-3-((4- (trifluoromethoxy)phenyl)sulfonamido) propanoic acid | 66.0 |
| 114 | 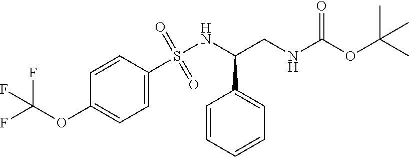 [SMILES: CC(C)(C)OC(=O)NC[C@H](NS(=O)(=O)c1ccc(OC(F)(F)F)cc1)c1ccccc1] | tert-butyl (R)-(2-phenyl-2-((4- (trifluoromethoxy)phenyl)sulfonamido) ethyl)carbamate | 42.5 |
| 115 | 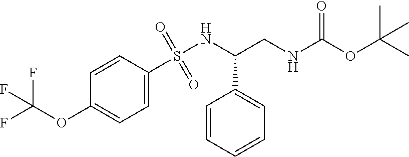 [SMILES: CC(C)(C)OC(=O)NC[C@@H](NS(=O)(=O)c1ccc(OC(F)(F)F)cc1)c1ccccc1] | tert-butyl (S)-(2-phenyl-2-((4- (trifluoromethoxy)phenyl)sulfonamido) ethyl)carbamate | 51.58 |
| 116 | 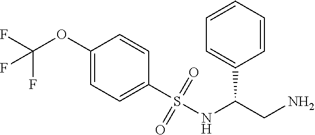 [SMILES: NC[C@H](NS(=O)(=O)c1ccc(OC(F)(F)F)cc1)c1ccccc1] | (R)-N-(2-amino-1-phenylethyl)-4- (trifluoromethoxy)benzenesulfonamide | 51.16 |
| 117 | 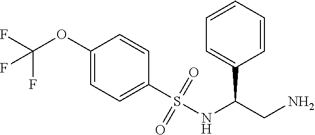 [SMILES: NC[C@@H](NS(=O)(=O)c1ccc(OC(F)(F)F)cc1)c1ccccc1] | (S)-N-(2-amino-1-phenylethyl)-4- (trifluoromethoxy)benzenesulfonamide | 55.0 |
| 118 | 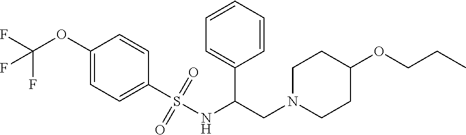 [SMILES: CCCOC1CCN(CC(NS(=O)(=O)c2ccc(OC(F)(F)F)cc2)c2ccccc2)CC1] | N-(1-phenyl-2-(4- propoxypiperidin-1-yl)ethyl)-4- (trifluoromethoxy)benzenesulfonamide | 42.42 |
| 119 | 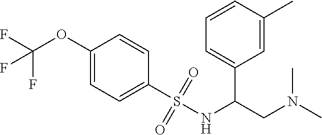 [SMILES: Cc1cccc(C(CN(C)C)NS(=O)(=O)c2ccc(OC(F)(F)F)cc2)c1] | N-(2-(dimethylamino)-1-(m- tolyl)ethyl)-4- (trifluoromethoxy)benzenesulfonamide | 48.51 |
| 120 | 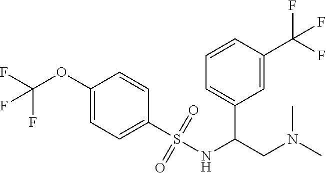 [SMILES: CN(C)CC(NS(=O)(=O)c1ccc(OC(F)(F)F)cc1)c1cccc(C(F)(F)F)c1] | N-(2-(dimethylamino)-1-(3- (trifluoromethyl)phenyl)ethyl)-4- (trifluoromethoxy)benzenesulfonamide | 46.29 |
| 121 | 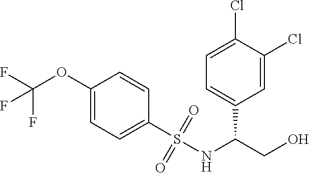 [SMILES: O=S(=O)(N[C@@H](CO)c1ccc(Cl)c(Cl)c1)c1ccc(OC(F)(F)F)cc1] | (R)-N-(1-(3,4-dichlorophenyl)-2- hydroxyethyl)-4- (trifluoromethoxy)benzenesulfonamide | 40.23 |
| 122 | 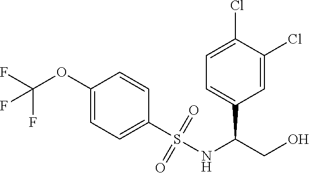 [SMILES: O=S(=O)(N[C@H](CO)c1ccc(Cl)c(Cl)c1)c1ccc(OC(F)(F)F)cc1] | (S)-N-(1-(3,4-dichlorophenyl)-2- hydroxyethyl)-4- (trifluoromethoxy)benzenesulfonamide | 43.42 |
| 123 | 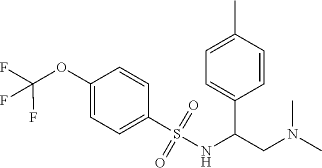 [SMILES: Cc1ccc(C(CN(C)C)NS(=O)(=O)c2ccc(OC(F)(F)F)cc2)cc1] | N-(2-(dimethylamino)-1-(p- tolyl)ethyl)-4- (trifluoromethoxy)benzenesulfonamide | 45.25 |
| 124 | 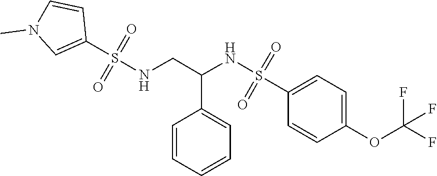 [SMILES: Cn1ccc(S(=O)(=O)NCC(NS(=O)(=O)c2ccc(OC(F)(F)F)cc2)c2ccccc2)c1] | 1-methyl-N-(2-phenyl-2-((4- (trifluoromethoxy)phenyl)sulfonamido) ethyl)-1H-pyrrole-3-sulfonamide | 49.73 |
| 125 | 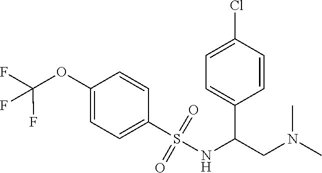 [SMILES: CN(C)CC(NS(=O)(=O)c1ccc(OC(F)(F)F)cc1)c1ccc(Cl)cc1] | N-(1-(4-chlorophenyl)-2- (dimethylamino)ethyl)-4- (trifluoromethoxy)benzenesulfonamide | 57.01 |
| 126 | 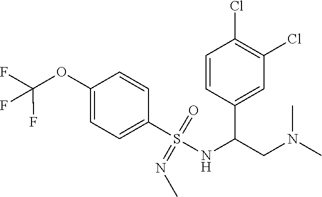 [SMILES: CN=S(=O)(NC(CN(C)C)c1ccc(Cl)c(Cl)c1)c1ccc(OC(F)(F)F)cc1] | N-(1-(3,4-dichlorophenyl)-2- (dimethylamino)ethyl)-N′-methyl-4- (trifluoromethoxy) benzenesulfonimidamide | 24.9 |
| 127 | 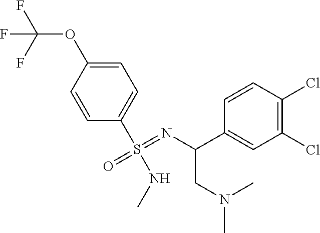 [SMILES: CNS(=O)(=NC(CN(C)C)c1ccc(Cl)c(Cl)c1)c1ccc(OC(F)(F)F)cc1] | N′-(1-(3,4-dichlorophenyl)-2- (dimethylamino)ethyl)-N-methyl-4- (trifluoromethoxy) benzenesulfonimidamide | 51.85 |
| 128 | 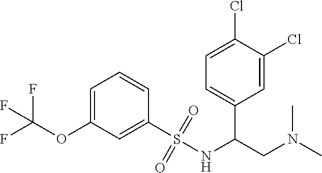 [SMILES: CN(C)CC(NS(=O)(=O)c1cccc(OC(F)(F)F)c1)c1ccc(Cl)c(Cl)c1] | N-(1-(3,4-dichlorophenyl)-2- (dimethylamino)ethyl)-3- (trifluoromethoxy)benzenesulfonamide | 21.13 |
| 129 | 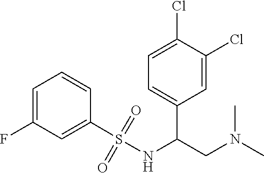 [SMILES: CN(C)CC(NS(=O)(=O)c1cccc(F)c1)c1ccc(Cl)c(Cl)c1] | N-(1-(3,4-dichlorophenyl)-2- (dimethylamino)ethyl)-3- fluorobenzenesulfonamide | 69.94 |
| 130 | 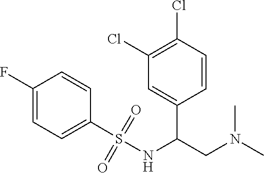 [SMILES: CN(C)CC(NS(=O)(=O)c1ccc(F)cc1)c1ccc(Cl)c(Cl)c1] | N-(1-(3,4-dichlorophenyl)-2- (dimethylamino)ethyl)-4- fluorobenzenesulfonamide | 62.43 |
| 131 | 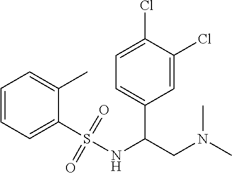 [SMILES: Cc1ccccc1S(=O)(=O)NC(CN(C)C)c1ccc(Cl)c(Cl)c1] | N-(1-(3,4-dichlorophenyl)-2- (dimethylamino)ethyl)-2- methylbenzenesulfonamide | 49.01 |
| 132 | 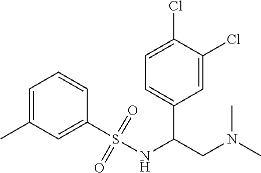 | N-(1-(3,4-dichlorophenyl)-2- (dimethylamino)ethyl)-3- methylbenzenesulfonamide | 40.49 |
| 133 | 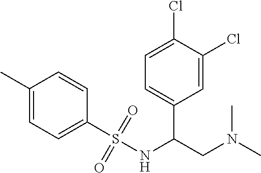 | N-(1-(3,4-dichlorophenyl)-2- (dimethylamino)ethyl)-4- methylbenzenesulfonamide | 51.84 |
| 134 | 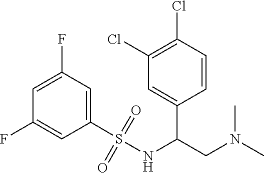 [SMILES: CN(C)CC(NS(=O)(=O)c1cc(F)cc(F)c1)c1ccc(Cl)c(Cl)c1] | N-(1-(3,4-dichlorophenyl)-2- (dimethylamino)ethyl)-3,5- difluorobenzenesulfonamide | 75.62 |
| 135 | 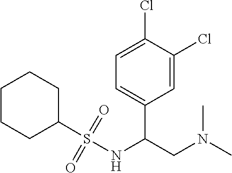 [SMILES: CN(C)CC(NS(=O)(=O)C1CCCCC1)c1ccc(Cl)c(Cl)c1] | N-(1-(3,4-dichlorophenyl)-2- (dimethylamino)ethyl) cyclohexanesulfonamide | 57.29 |
| 136 | 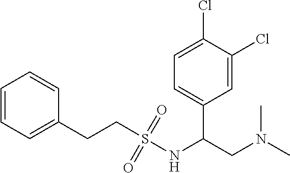 [SMILES: CN(C)CC(NS(=O)(=O)CCc1ccccc1)c1ccc(Cl)c(Cl)c1] | N-(1-(3,4-dichlorophenyl)-2- (dimethylamino)ethyl)-2- phenylethane-1-sulfonamide | 24.32 |
| 137 | 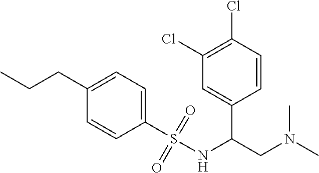 [SMILES: CCCc1ccc(S(=O)(=O)NC(CN(C)C)c2ccc(Cl)c(Cl)c2)cc1] | N-(1-(3,4-dichlorophenyl)-2- (dimethylamino)ethyl)-4- propylbenzenesulfonamide | 12.27 |
| 138 | 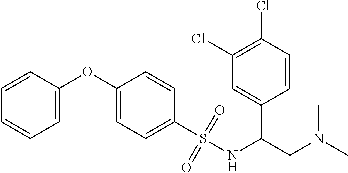 [SMILES: CN(C)CC(NS(=O)(=O)c1ccc(Oc2ccccc2)cc1)c1ccc(Cl)c(Cl)c1] | N-(1-(3,4-dichlorophenyl)-2- (dimethylamino)ethyl)-4- phenoxybenzenesulfonamide | 10.48 |
| 139 | 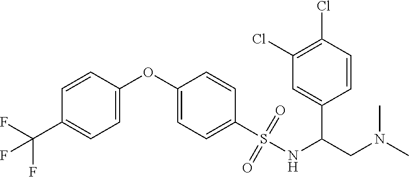 [SMILES: CN(C)CC(NS(=O)(=O)c1ccc(Oc2ccc(C(F)(F)F)cc2)cc1)c1ccc(Cl)c(Cl)c1] | N-(1-(3,4-dichlorophenyl)-2- (dimethylamino)ethyl)-4-(4- (trifluoromethyl)phenoxy) benzenesulfonamide | 10.19 |
| 140 | 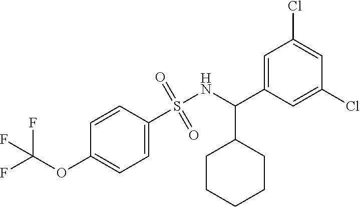 [SMILES: O=S(=O)(NC(c1cc(Cl)cc(Cl)c1)C1CCCCC1)c1ccc(OC(F)(F)F)cc1] | N-(cyclohexyl(3,5- dichlorophenyl)methyl)-4- (trifluoromethoxy)benzenesulfonamide enantiomer I | 19.04 |
| 141 | 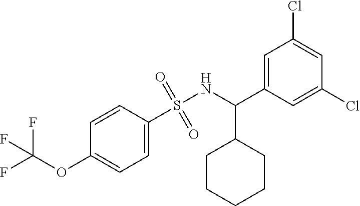 [SMILES: O=S(=O)(NC(c1cc(Cl)cc(Cl)c1)C1CCCCC1)c1ccc(OC(F)(F)F)cc1] | N-(cyclohexyl(3,5- dichlorophenyl)methyl)-4- (trifluoromethoxy)benzenesulfonamide enantiomer II | 21.3 |
| 142a | 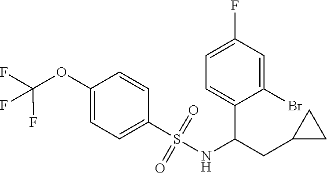 [SMILES: O=S(=O)(NC(CC1CC1)c1ccc(F)cc1Br)c1ccc(OC(F)(F)F)cc1] | N-(1-(2-bromo-4-fluorophenyl)-2- cyclopropylethyl)-4- (trifluoromethoxy)benzenesulfonamide enantiomer I | 80 |
| 142b | 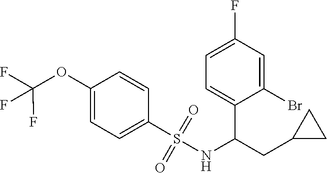 [SMILES: O=S(=O)(NC(CC1CC1)c1ccc(F)cc1Br)c1ccc(OC(F)(F)F)cc1] | N-(1-(2-bromo-4-fluorophenyl)-2- cyclopropylethyl)-4- (trifluoromethoxy)benzenesulfonamide enantiomer II | 49.61 |
| 143 | 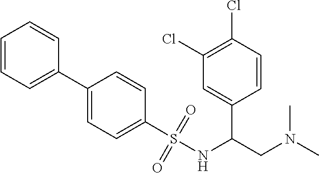 [SMILES: CN(C)CC(NS(=O)(=O)c1ccc(-c2ccccc2)cc1)c1ccc(Cl)c(Cl)c1] | N-(1-(3,4-dichlorophenyl)-2- (dimethylamino)ethyl)-[1,1′- biphenyl]-4-sulfonamide | 11.23 |
| 144 | 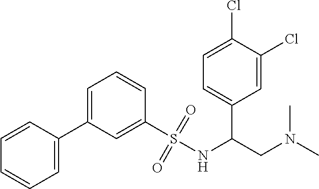 [SMILES: CN(C)CC(NS(=O)(=O)c1cccc(-c2ccccc2)c1)c1ccc(Cl)c(Cl)c1] | N-(1-(3,4-dichlorophenyl)-2- (dimethylamino)ethyl)-[1,1′- biphenyl]-3-sulfonamide | 14.44 |
| 145 | 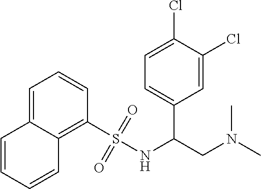 [SMILES: CN(C)CC(NS(=O)(=O)c1cccc2ccccc12)c1ccc(Cl)c(Cl)c1] | N-(1-(3,4-dichlorophenyl)-2- (dimethylamino)ethyl)naphthalene-1- sulfonamide | 21.73 |
| 146 | 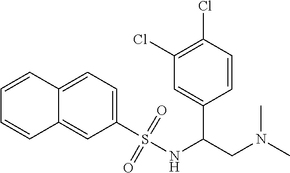 [SMILES: CN(C)CC(NS(=O)(=O)c1ccc2ccccc2c1)c1ccc(Cl)c(Cl)c1] | N-(1-(3,4-dichlorophenyl)-2- (dimethylamino)ethyl)naphthalene-2- sulfonamide | 23.77 |
| 147 | 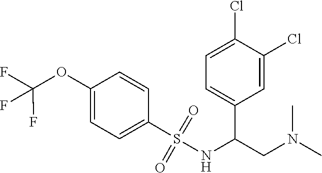 [SMILES: CN(C)CC(NS(=O)(=O)c1ccc(OC(F)(F)F)cc1)c1ccc(Cl)c(Cl)c1] | N-(1-(3,4-dichlorophenyl)-2- (dimethylamino)ethyl)-4- (trifluoromethoxy)benzenesulfonamide | 19.5 |
| 148 | 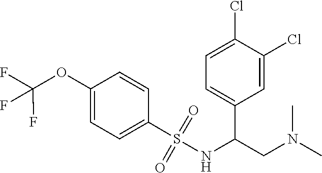 [SMILES: CN(C)CC(NS(=O)(=O)c1ccc(OC(F)(F)F)cc1)c1ccc(Cl)c(Cl)c1] | N-(1-(3,4-dichlorophenyl)-2- (dimethylamino)ethyl)-4- (trifluoromethoxy)benzenesulfonamide enantiomer I | 12.7 |
| 149 | 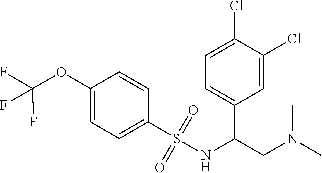 [SMILES: CN(C)CC(NS(=O)(=O)c1ccc(OC(F)(F)F)cc1)c1ccc(Cl)c(Cl)c1] | N-(1-(3,4-dichlorophenyl)-2- (dimethylamino)ethyl)-4- (trifluoromethoxy)benzenesulfonamide enantiomer II | 23.9 |
| 150 | 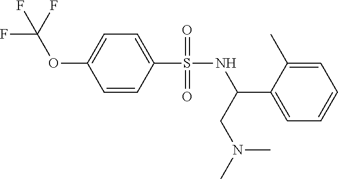 [SMILES: Cc1ccccc1C(CN(C)C)NS(=O)(=O)c1ccc(OC(F)(F)F)cc1] | N-(2-(dimethylamino)-1-(o- tolyl)ethyl)-4- (trifluoromethoxy)benzenesulfonamide | 59.56 |
| 151 | 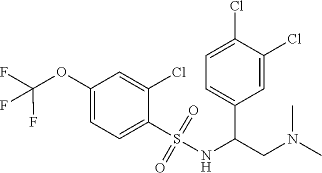 [SMILES: CN(C)CC(NS(=O)(=O)c1ccc(OC(F)(F)F)cc1Cl)c1ccc(Cl)c(Cl)c1] | 2-chloro-N-(1-(3,4- dichlorophenyl)-2- (dimethylamino)ethyl)-4- (trifluoromethoxy)benzenesulfonamide | 23.62 |
| 152 | 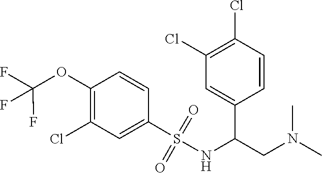 [SMILES: CN(C)CC(NS(=O)(=O)c1ccc(OC(F)(F)F)c(Cl)c1)c1ccc(Cl)c(Cl)c1] | 3-chloro-N-(1-(3,4- dichlorophenyl)-2- (dimethylamino)ethyl)-4- (trifluoromethoxy)benzenesulfonamide | 12.11 |
| 153 | 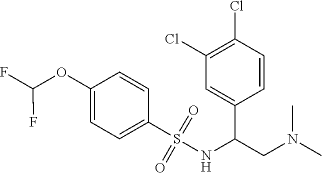 [SMILES: CN(C)CC(NS(=O)(=O)c1ccc(OC(F)F)cc1)c1ccc(Cl)c(Cl)c1] | N-(1-(3,4-dichlorophenyl)-2- (dimethylamino)ethyl)-4- (difluoromethoxy)benzenesulfonamide | 45.73 |
| 154 | 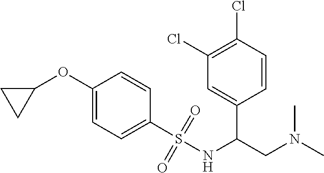 [SMILES: CN(C)CC(NS(=O)(=O)c1ccc(OC2CC2)cc1)c1ccc(Cl)c(Cl)c1] | 4-cyclopropoxy-N-(1-(3,4- dichlorophenyl)-2- (dimethylamino)ethyl) benzenesulfonamide | 53.77 |
| 155 | 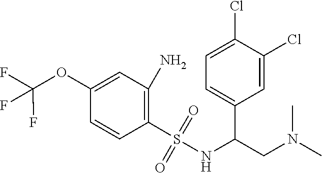 [SMILES: CN(C)CC(NS(=O)(=O)c1ccc(OC(F)(F)F)cc1N)c1ccc(Cl)c(Cl)c1] | 2-amino-N-(1-(3,4- dichlorophenyl)-2- (dimethylamino)ethyl)-4- (trifluoromethoxy)benzenesulfonamide | 21.27 |
| 156 | 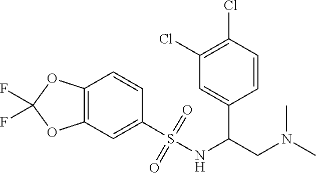 [SMILES: CN(C)CC(NS(=O)(=O)c1ccc2c(c1)OC(F)(F)O2)c1ccc(Cl)c(Cl)c1] | N-(1-(3,4-dichlorophenyl)-2- (dimethylamino)ethyl)-2,2- difluorobenzo[d][1,3]dioxole-5- sulfonamide | 24.9 |
| 157 | 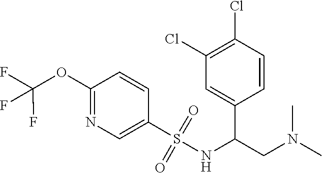 [SMILES: CN(C)CC(NS(=O)(=O)c1ccc(OC(F)(F)F)nc1)c1ccc(Cl)c(Cl)c1] | N-(1-(3,4-dichlorophenyl)-2- (dimethylamino)ethyl)-6- (trifluoromethoxy)pyridine-2- sulfonamide | 17.65 |
| 158 | 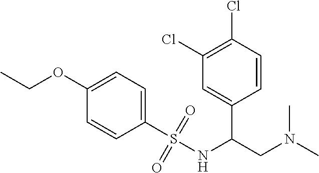 [SMILES: CCOc1ccc(S(=O)(=O)NC(CN(C)C)c2ccc(Cl)c(Cl)c2)cc1] | N-(1-(3,4-dichlorophenyl)-2- (dimethylamino)ethyl)-4- ethoxybenzenesulfonamide | 51.28 |
| 158 | 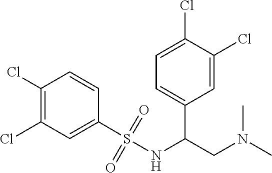 [SMILES: CN(C)CC(NS(=O)(=O)c1ccc(Cl)c(Cl)c1)c1ccc(Cl)c(Cl)c1] | 3,4-dichloro-N-(1-(3,4- dichlorophenyl)-2- (dimethylamino)ethyl) benzenesulfonamide | 23.97 |
| 160 | 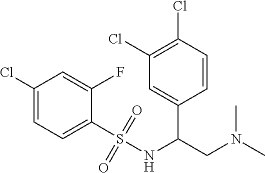 [SMILES: CN(C)CC(NS(=O)(=O)c1ccc(Cl)cc1F)c1ccc(Cl)c(Cl)c1] | 4-chloro-N-(1-(3,4- dichlorophenyl)-2- (dimethylamino)ethyl)-2- fluorobenzenesulfonamide | 58.47 |
| 161 | 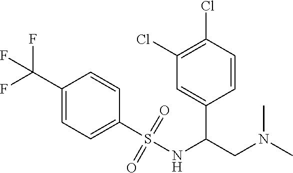 [SMILES: CN(C)CC(NS(=O)(=O)c1ccc(C(F)(F)F)cc1)c1ccc(Cl)c(Cl)c1] | N-(1-(3,4-dichlorophenyl)-2- (dimethylamino)ethyl)-4- (trifluoromethyl)benzenesulfonamide | 25.37 |
| 162 | 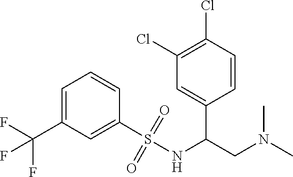 [SMILES: CN(C)CC(NS(=O)(=O)c1cccc(C(F)(F)F)c1)c1ccc(Cl)c(Cl)c1] | N-(1-(3,4-dichlorophenyl)-2- (dimethylamino)ethyl)-3- (trifluoromethyl)benzenesulfonamide | 52.68 |
| 163 | 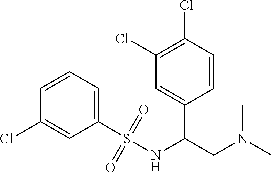 [SMILES: CN(C)CC(NS(=O)(=O)c1cccc(Cl)c1)c1ccc(Cl)c(Cl)c1] | 3-chloro-N-(1-(3,4- dichlorophenyl)-2- (dimethylamino)ethyl) benzenesulfonamide | 46.02 |
| 164 | 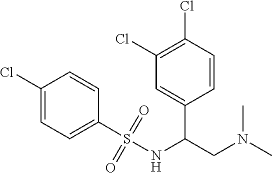 [SMILES: CN(C)CC(NS(=O)(=O)c1ccc(Cl)cc1)c1ccc(Cl)c(Cl)c1] | 4-chloro-N-(1-(3,4- dichlorophenyl)-2- (dimethylamino)ethyl) benzenesulfonamide | 28.22 |
| 165a | 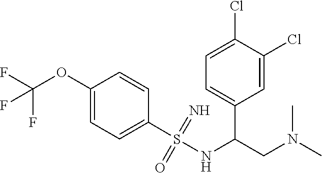 [SMILES: CN(C)CC(NS(=N)(=O)c1ccc(OC(F)(F)F)cc1)c1ccc(Cl)c(Cl)c1] | N-(1-(3,4-dichlorophenyl)-2- (dimethylamino)ethyl)-4- (trifluoromethoxy) benzenesulfonimidamide diastereomer I (racemic) | 46.3 |
| 165b | 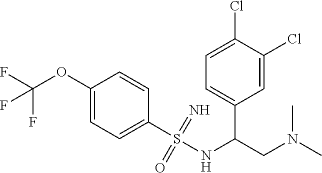 [SMILES: CN(C)CC(NS(=N)(=O)c1ccc(OC(F)(F)F)cc1)c1ccc(Cl)c(Cl)c1] | N-(1-(3,4-dichlorophenyl)-2- (dimethylamino)ethyl)-4- (trifluoromethoxy) benzenesulfonimidamide diastereomer II (racemic) | 53.75 |
| 166 | 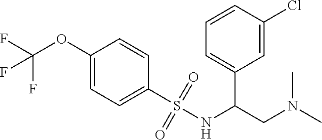 [SMILES: CN(C)CC(NS(=O)(=O)c1ccc(OC(F)(F)F)cc1)c1cccc(Cl)c1] | N-(1-(3-chlorophenyl)-2- (dimethylamino)ethyl)-4- (trifluoromethoxy)benzenesulfonamide | 44.45 |
| 167 | 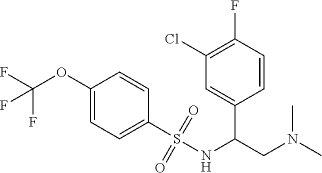 [SMILES: CN(C)CC(NS(=O)(=O)c1ccc(OC(F)(F)F)cc1)c1ccc(F)c(Cl)c1] | N-(1-(3-chloro-4-fluorophenyl)-2- (dimethylamino)ethyl)-4- (trifluoromethoxy)benzenesulfonamide | 42.41 |
| 168 | 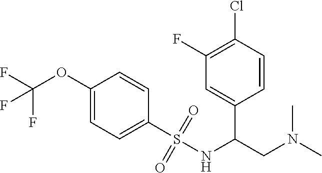 [SMILES: CN(C)CC(NS(=O)(=O)c1ccc(OC(F)(F)F)cc1)c1ccc(Cl)c(F)c1] | N-(1-(4-chloro-3-fluorophenyl)-2- (dimethylamino)ethyl)-4- (trifluoromethoxy)benzenesulfonamide | 24.65 |
| 169 | 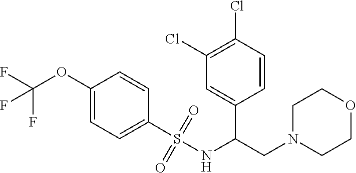 [SMILES: O=S(=O)(NC(CN1CCOCC1)c1ccc(Cl)c(Cl)c1)c1ccc(OC(F)(F)F)cc1] | N-(1-(3,4-dichlorophenyl)-2- morpholinoethyl)-4- (trifluoromethoxy)benzenesulfonamide | 53.66 |
| 170 | 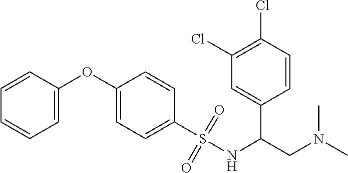 [SMILES: CN(C)CC(NS(=O)(=O)c1ccc(Oc2ccccc2)cc1)c1ccc(Cl)c(Cl)c1] | N-(1-(3,4-dichlorophenyl)-2- (dimethylamino)ethyl)-4- phenoxybenzenesulfonamide enantiomer I | 13.33 |
| 171 | 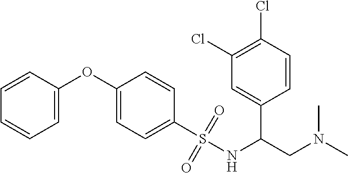 [SMILES: CN(C)CC(NS(=O)(=O)c1ccc(Oc2ccccc2)cc1)c1ccc(Cl)c(Cl)c1] | N-(1-(3,4-dichlorophenyl)-2- (dimethylamino)ethyl)-4- phenoxybenzenesulfonamide enantiomer II | 15.8 |
| 172 | 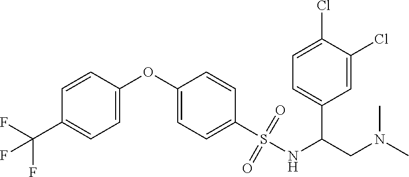 [SMILES: CN(C)CC(NS(=O)(=O)c1ccc(Oc2ccc(C(F)(F)F)cc2)cc1)c1ccc(Cl)c(Cl)c1] | N-(1-(3,4-dichlorophenyl)-2- (dimethylamino)ethyl)-4-(4- (trifluoromethyl)phenoxy) benzenesulfonamide enantiomer I | 6.04 |
| 173 | 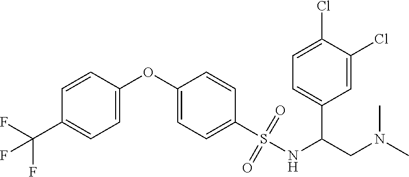 [SMILES: CN(C)CC(NS(=O)(=O)c1ccc(Oc2ccc(C(F)(F)F)cc2)cc1)c1ccc(Cl)c(Cl)c1] | N-(1-(3,4-dichlorophenyl)-2- (dimethylamino)ethyl)-4-(4- (trifluoromethyl)phenoxy) benzenesulfonamide enantiomer II | 12.96 |
| 174 | 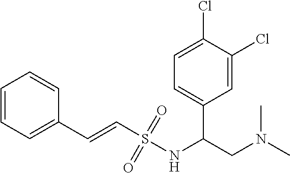 [SMILES: CN(C)CC(NS(=O)(=O)/C=C/c1ccccc1)c1ccc(Cl)c(Cl)c1] | (E)-N-(1-(3,4-dichlorophenyl)-2- (dimethylamino)ethyl)-2- phenylethene-1-sulfonamide | 30.87 |
| 175 | 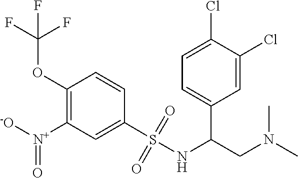 | N-(1-(3,4-dichlorophenyl)-2- (dimethylamino)ethyl)-3-nitro-4- (trifluoromethoxy)benzenesulfonamide | 3.227 |
| 176 | 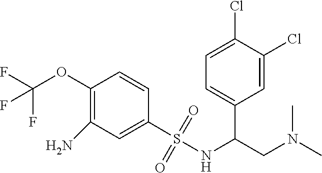 [SMILES: CN(C)CC(NS(=O)(=O)c1ccc(OC(F)(F)F)c(N)c1)c1ccc(Cl)c(Cl)c1] | 3-amino-N-(1-(3,4- dichlorophenyl)-2- (dimethylamino)ethyl)-4- (trifluoromethoxy)benzenesulfonamide | 31.42 |
| 177 | 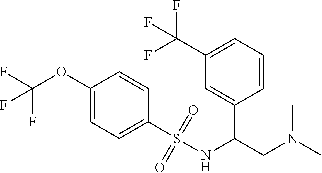 [SMILES: CN(C)CC(NS(=O)(=O)c1ccc(OC(F)(F)F)cc1)c1cccc(C(F)(F)F)c1] | N-(2-(dimethylamino)-1-(3- (trifluoromethyl)phenyl)ethyl)-4- (trifluoromethoxy)benzenesulfonamide enantiomer I | 24.92 |
| 178 | 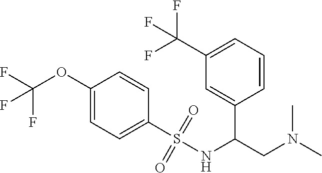 [SMILES: CN(C)CC(NS(=O)(=O)c1ccc(OC(F)(F)F)cc1)c1cccc(C(F)(F)F)c1] | N-(2-(dimethylamino)-1-(3- (trifluoromethyl)phenyl)ethyl)-4- (trifluoromethoxy)benzenesulfonamide enantiomer II | 51.16 |
| 179 | 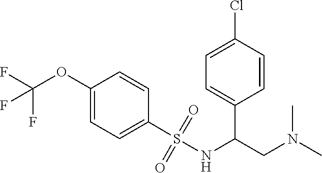 [SMILES: CN(C)CC(NS(=O)(=O)c1ccc(OC(F)(F)F)cc1)c1ccc(Cl)cc1] | N-(1-(4-chlorophenyl)-2- (dimethylamino)ethyl)-4- (trifluoromethoxy)benzenesulfonamide enantiomer I | 43.06 |
| 180 | 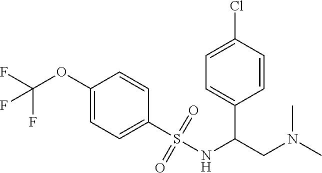 [SMILES: CN(C)CC(NS(=O)(=O)c1ccc(OC(F)(F)F)cc1)c1ccc(Cl)cc1] | N-(1-(4-chlorophenyl)-2- (dimethylamino)ethyl)-4- (trifluoromethoxy)benzenesulfonamide enantiomer II | 51.27 |
| 181 | 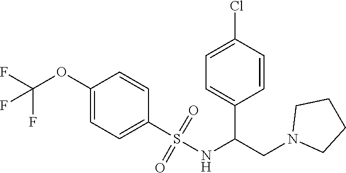 [SMILES: O=S(=O)(NC(CN1CCCC1)c1ccc(Cl)cc1)c1ccc(OC(F)(F)F)cc1] | N-(1-(4-chlorophenyl)-2- (pyrrolidin-1-yl)ethyl)-4- (trifluoromethoxy)benzenesulfonamide | 51.37 |
| 182 | 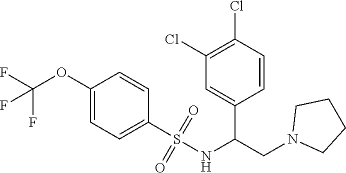 [SMILES: O=S(=O)(NC(CN1CCCC1)c1ccc(Cl)c(Cl)c1)c1ccc(OC(F)(F)F)cc1] | N-(1-(3,4-dichlorophenyl)-2- (pyrrolidin-1-yl)ethyl)-4- (trifluoromethoxy)benzenesulfonamide | 24.46 |
| 183 | 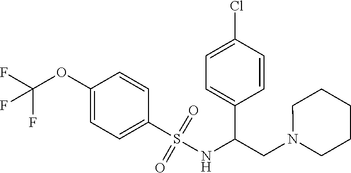 [SMILES: O=S(=O)(NC(CN1CCCCC1)c1ccc(Cl)cc1)c1ccc(OC(F)(F)F)cc1] | N-(1-(4-chlorophenyl)-2- (piperidin-1-yl)ethyl)-4- (trifluoromethoxy)benzenesulfonamide | 33.82 |
| 184 | 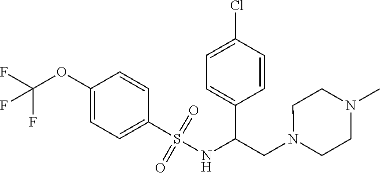 [SMILES: CN1CCN(CC(NS(=O)(=O)c2ccc(OC(F)(F)F)cc2)c2ccc(Cl)cc2)CC1] | N-(1-(4-chlorophenyl)-2-(4- methylpiperazin-1-yl)ethyl)-4- (trifluoromethoxy)benzenesulfonamide | 23.42 |
| 185 | 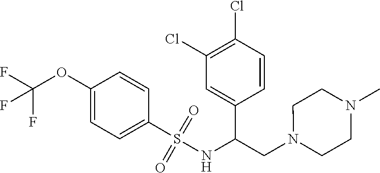 [SMILES: CN1CCN(CC(NS(=O)(=O)c2ccc(OC(F)(F)F)cc2)c2ccc(Cl)c(Cl)c2)CC1] | N-(1-(3,4-dichlorophenyl)-2-(4- methylpiperazin-1-yl)ethyl)-4- (trifluoromethoxy)benzenesulfonamide | 12.62 |
| 186 | 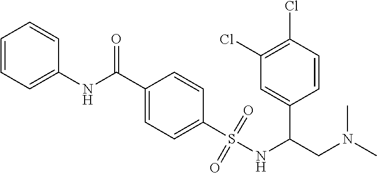 [SMILES: CN(C)CC(NS(=O)(=O)c1ccc(C(=O)Nc2ccccc2)cc1)c1ccc(Cl)c(Cl)c1] | 4-(N-(1-(3,4-dichlorophenyl)-2- (dimethylamino)ethyl)sulfamoyl)- N-phenylbenzamide | 48.25 |
| 187 | 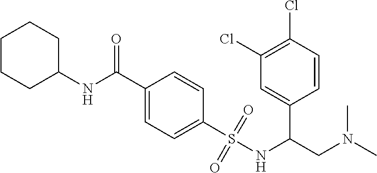 [SMILES: CN(C)CC(NS(=O)(=O)c1ccc(C(=O)NC2CCCCC2)cc1)c1ccc(Cl)c(Cl)c1] | N-cyclohexyl-4-(N-(1-(3,4- dichlorophenyl)-2- (dimethylamino)ethyl)sulfamoyl) benzamide | 53.63 |
| 188 | 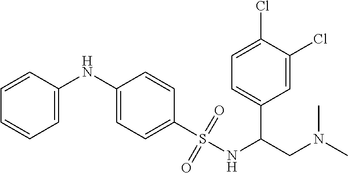 [SMILES: CN(C)CC(NS(=O)(=O)c1ccc(Nc2ccccc2)cc1)c1ccc(Cl)c(Cl)c1] | N-(1-(3,4-dichlorophenyl)-2- (dimethylamino)ethyl)-4- (phenylamino)benzenesulfonamide | 13.6 |
| 189 | 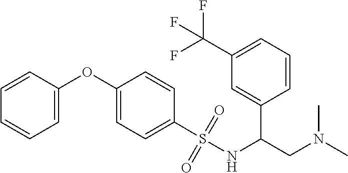 [SMILES: CN(C)CC(NS(=O)(=O)c1ccc(Oc2ccccc2)cc1)c1cccc(C(F)(F)F)c1] | N-(2-(dimethylamino)-1-(3- (trifluoromethyl)phenyl)ethyl)-4- phenoxybenzenesulfonamide | 12.06 |
| 190 | 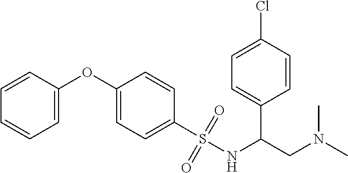 [SMILES: CN(C)CC(NS(=O)(=O)c1ccc(Oc2ccccc2)cc1)c1ccc(Cl)cc1] | N-(1-(4-chlorophenyl)-2- (dimethylamino)ethyl)-4- phenoxybenzenesulfonamide | 11.78 |
| 191 | 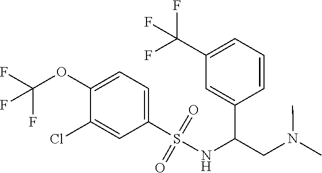 [SMILES: CN(C)CC(NS(=O)(=O)c1ccc(OC(F)(F)F)c(Cl)c1)c1cccc(C(F)(F)F)c1] | 3-chloro-N-(2-(dimethylamino)-1- (3-(trifluoromethyl)phenyl)ethyl)-4- (trifluoromethoxy)benzenesulfonamide | 16.48 |
| 192 | 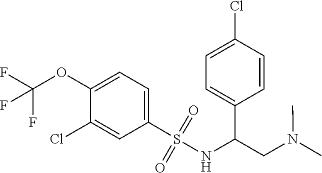 [SMILES: CN(C)CC(NS(=O)(=O)c1ccc(OC(F)(F)F)c(Cl)c1)c1ccc(Cl)cc1] | 3-chloro-N-(1-(4-chlorophenyl)-2- (dimethylamino)ethyl)-4- (trifluoromethoxy)benzenesulfonamide | 14.44 |
| 193 | 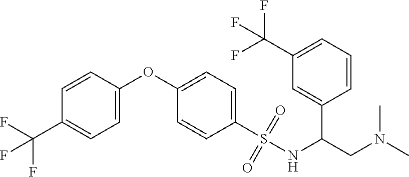 [SMILES: CN(C)CC(NS(=O)(=O)c1ccc(Oc2ccc(C(F)(F)F)cc2)cc1)c1cccc(C(F)(F)F)c1] | N-(2-(dimethylamino)-1-(3- (trifluoromethyl)phenyl)ethyl)-4- (4-(trifluoromethyl)phenoxy) benzenesulfonamide | 19.55 |
| 194 | 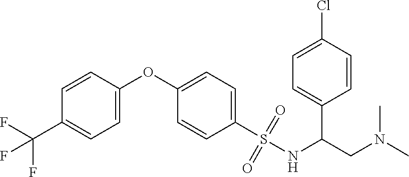 [SMILES: CN(C)CC(NS(=O)(=O)c1ccc(Oc2ccc(C(F)(F)F)cc2)cc1)c1ccc(Cl)cc1] | N-(1-(4-chlorophenyl)-2- (dimethylamino)ethyl)-4-(4- (trifluoromethyl)phenoxy) benzenesulfonamide | 12.16 |
| 195 | 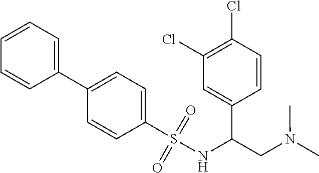 [SMILES: CN(C)CC(NS(=O)(=O)c1ccc(-c2ccccc2)cc1)c1ccc(Cl)c(Cl)c1] | N-(1-(3,4-dichlorophenyl)-2- (dimethylamino)ethyl)-[1,1′- biphenyl]-4-sulfonamide enantiomer I | 11.89 |
| 196 | 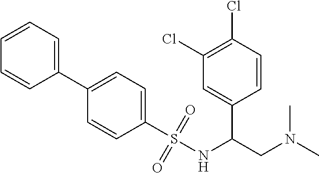 [SMILES: CN(C)CC(NS(=O)(=O)c1ccc(-c2ccccc2)cc1)c1ccc(Cl)c(Cl)c1] | N-(1-(3,4-dichlorophenyl)-2- (dimethylamino)ethyl)-[1,1′- biphenyl]-4-sulfonamide enantiomer II | 15.88 |
| 197 | 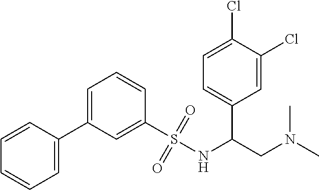 [SMILES: CN(C)CC(NS(=O)(=O)c1cccc(-c2ccccc2)c1)c1ccc(Cl)c(Cl)c1] | N-(1-(3,4-dichlorophenyl)-2- (dimethylamino)ethyl)-[1,1′- biphenyl]-3-sulfonamide enantiomer I | 19.33 |
| 198 | 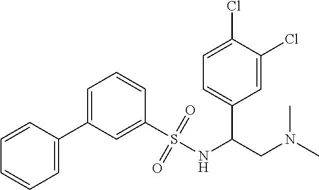 [SMILES: CN(C)CC(NS(=O)(=O)c1cccc(-c2ccccc2)c1)c1ccc(Cl)c(Cl)c1] | N-(1-(3,4-dichlorophenyl)-2- (dimethylamino)ethyl)-[1,1′- biphenyl]-3-sulfonamide enantiomer II | 12.54 |
| 199 | 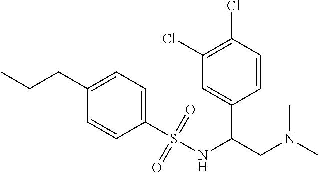 [SMILES: CCCc1ccc(S(=O)(=O)NC(CN(C)C)c2ccc(Cl)c(Cl)c2)cc1] | N-(1-(3,4-dichlorophenyl)-2- (dimethylamino)ethyl)-4- propylbenzenesulfonamide enantiomer I | 14.43 |
| 200 | 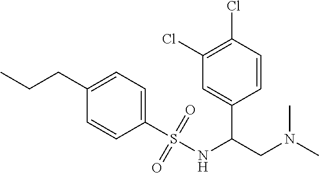 [SMILES: CCCc1ccc(S(=O)(=O)NC(CN(C)C)c2ccc(Cl)c(Cl)c2)cc1] | N-(1-(3,4-dichlorophenyl)-2- (dimethylamino)ethyl)-4- propylbenzenesulfonamide enantiomer II | 17.79 |
| 201 | 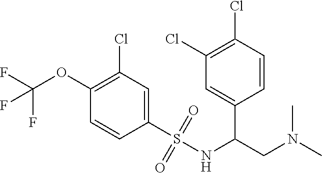 [SMILES: CN(C)CC(NS(=O)(=O)c1ccc(OC(F)(F)F)c(Cl)c1)c1ccc(Cl)c(Cl)c1] | 3-chloro-N-(1-(3,4- dichlorophenyl)-2- (dimethylamino)ethyl)-4- (trifluoromethoxy)benzenesulfonamide enantiomer I | 22.82 |
| 202 | 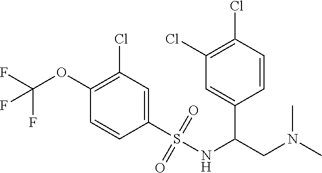 [SMILES: CN(C)CC(NS(=O)(=O)c1ccc(OC(F)(F)F)c(Cl)c1)c1ccc(Cl)c(Cl)c1] | 3-chloro-N-(1-(3,4- dichlorophenyl)-2- (dimethylamino)ethyl)-4- (trifluoromethoxy)benzenesulfonamide enantiomer II | 21.62 |
| 203 | 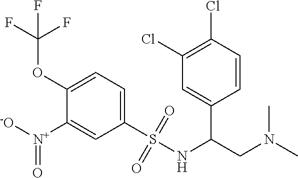 | N-(1-(3,4-dichlorophenyl)-2- (dimethylamino)ethyl)-3-nitro-4- (trifluoromethoxy)benzenesulfonamide enantiomer I | 1.397 |
| 204 | 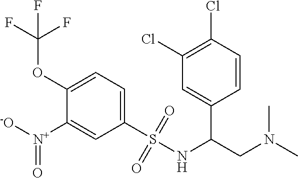 | N-(1-(3,4-dichlorophenyl)-2- (dimethylamino)ethyl)-3-nitro-4- (trifluoromethoxy)benzenesulfonamide enantiomer II | 1.143 |
| 205 | 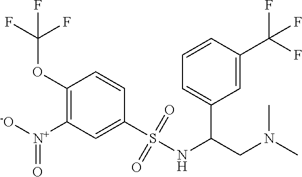 | N-(2-(dimethylamino)-1-(3- (trifluoromethyl)phenyl)ethyl)-3- nitro-4- (trifluoromethoxy)benzenesulfonamide | 2.265 |
| 206 | 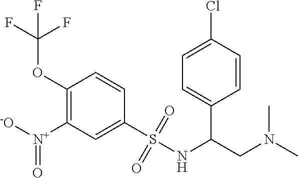 | N-(1-(4-chlorophenyl)-2- (dimethylamino)ethyl)-3-nitro-4- (trifluoromethoxy)benzenesulfonamide | 1.766 |
| 207 | 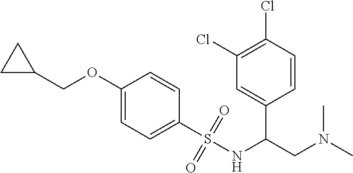 [SMILES: CN(C)CC(NS(=O)(=O)c1ccc(OCC2CC2)cc1)c1ccc(Cl)c(Cl)c1] | 4-(cyclopropylmethoxy)-N-(1- (3,4-dichlorophenyl)-2- (dimethylamino)ethyl) benzenesulfonamide | 21.8 |
| 208 | 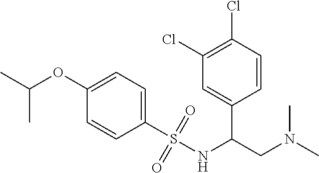 [SMILES: CC(C)Oc1ccc(S(=O)(=O)NC(CN(C)C)c2ccc(Cl)c(Cl)c2)cc1] | N-(1-(3,4-dichlorophenyl)-2- (dimethylamino)ethyl)-4- isopropoxybenzenesulfonamide | 25.42 |
| 209 | 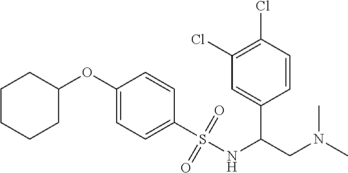 [SMILES: CN(C)CC(NS(=O)(=O)c1ccc(OC2CCCCC2)cc1)c1ccc(Cl)c(Cl)c1] | 4-(cyclohexyloxy)-N-(1-(3,4- dichlorophenyl)-2- (dimethylamino)ethyl) benzenesulfonamide | 11.14 |
| 210 | 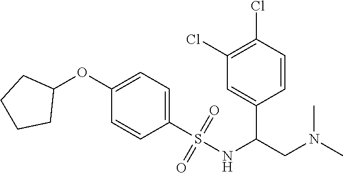 [SMILES: CN(C)CC(NS(=O)(=O)c1ccc(OC2CCCC2)cc1)c1ccc(Cl)c(Cl)c1] | 4-(cyclopentyloxy)-N-(1-(3,4- dichlorophenyl)-2- (dimethylamino)ethyl) benzenesulfonamide | 12.46 |
| 211 | 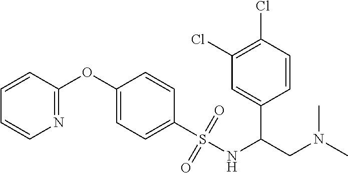 [SMILES: CN(C)CC(NS(=O)(=O)c1ccc(Oc2ccccn2)cc1)c1ccc(Cl)c(Cl)c1] | N-(1-(3,4-dichlorophenyl)-2- (dimethylamino)ethyl)-4-(pyridin- 2-yloxy)benzenesulfonamide | 75.0 |
| 212 | 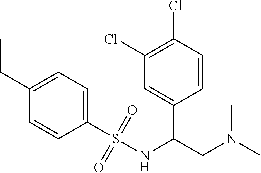 [SMILES: CCc1ccc(S(=O)(=O)NC(CN(C)C)c2ccc(Cl)c(Cl)c2)cc1] | N-(1-(3,4-dichlorophenyl)-2- (dimethylamino)ethyl)-4- ethylbenzenesulfonamide | 34.61 |
| 213 | 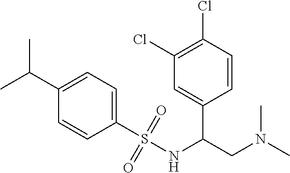 [SMILES: CC(C)c1ccc(S(=O)(=O)NC(CN(C)C)c2ccc(Cl)c(Cl)c2)cc1] | N-(1-(3,4-dichlorophenyl)-2- (dimethylamino)ethyl)-4- isopropylbenzenesulfonamide | 25.17 |
| 214 | 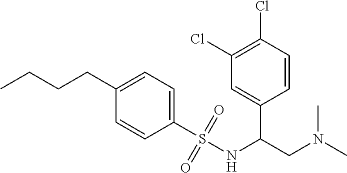 [SMILES: CCCCc1ccc(S(=O)(=O)NC(CN(C)C)c2ccc(Cl)c(Cl)c2)cc1] | 4-butyl-N-(1-(3,4-dichlorophenyl)- 2- (dimethylamino)ethyl) benzenesulfonamide | 11.34 |
| 215 | 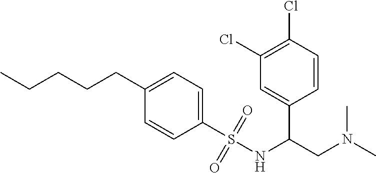 [SMILES: CCCCCc1ccc(S(=O)(=O)NC(CN(C)C)c2ccc(Cl)c(Cl)c2)cc1] | N-(1-(3,4-dichlorophenyl)-2- (dimethylamino)ethyl)-4- pentylbenzenesulfonamide | 7.504 |
| 216 | 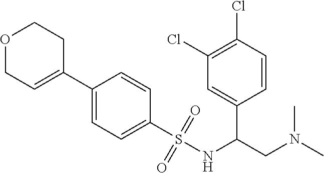 [SMILES: CN(C)CC(NS(=O)(=O)c1ccc(C2=CCOCC2)cc1)c1ccc(Cl)c(Cl)c1] | N-(1-(3,4-dichlorophenyl)-2- (dimethylamino)ethyl)-4-(3,6- dihydro-2H-pyran-4- yl)benzenesulfonamide | 8.179 |
| 217 | 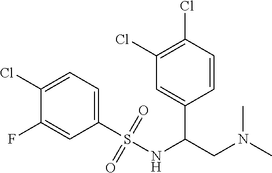 [SMILES: CN(C)CC(NS(=O)(=O)c1ccc(Cl)c(F)c1)c1ccc(Cl)c(Cl)c1] | 4-chloro-N-(1-(3,4- dichlorophenyl)-2- (dimethylamino)ethyl)-3- fluorobenzenesulfonamide | 49.5 |
| 218 | 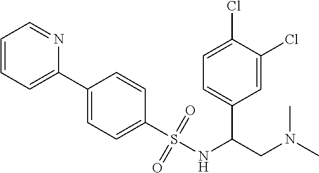 [SMILES: CN(C)CC(NS(=O)(=O)c1ccc(-c2ccccn2)cc1)c1ccc(Cl)c(Cl)c1] | N-(1-(3,4-dichlorophenyl)-2- (dimethylamino)ethyl)-4-(pyridin- 2-yl)benzenesulfonamide | 43.72 |
| 219 | 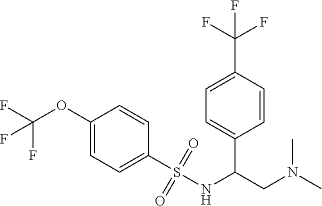 [SMILES: CN(C)CC(NS(=O)(=O)c1ccc(OC(F)(F)F)cc1)c1ccc(C(F)(F)F)cc1] | N-(2-(dimethylamino)-1-(4- (trifluoromethyl)phenyl)ethyl)-4- (trifluoromethoxy)benzenesulfonamide | 43.61 |
| 220 | 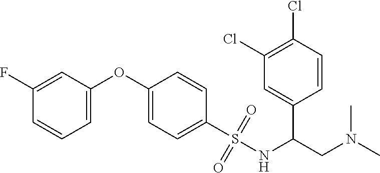 [SMILES: CN(C)CC(NS(=O)(=O)c1ccc(Oc2cccc(F)c2)cc1)c1ccc(Cl)c(Cl)c1] | N-(1-(3,4-dichlorophenyl)-2- (dimethylamino)ethyl)-4-(3- fluorophenoxy)benzenesulfonamide | 21.12 |
| 221 | 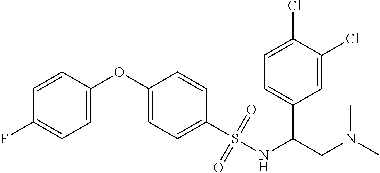 [SMILES: CN(C)CC(NS(=O)(=O)c1ccc(Oc2ccc(F)cc2)cc1)c1ccc(Cl)c(Cl)c1] | N-(1-(3,4-dichlorophenyl)-2- (dimethylamino)ethyl)-4-(4- fluorophenoxy)benzenesulfonamide | 26.61 |
| 222 | 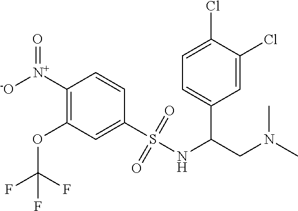 | N-(1-(3,4-dichlorophenyl)-2- (dimethylamino)ethyl)-4-nitro-3- (trifluoromethoxy)benzenesulfonamide | 1.339 |
| 223 | 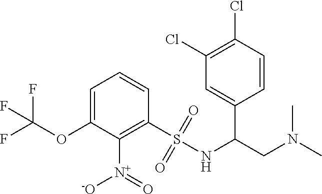 | N-(1-(3,4-dichlorophenyl)-2- (dimethylamino)ethyl)-2-nitro-3- (trifluoromethoxy)benzenesulfonamide | 57.4 |
| 224 | 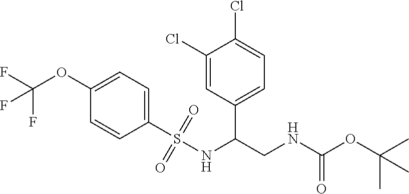 [SMILES: CC(C)(C)OC(=O)NCC(NS(=O)(=O)c1ccc(OC(F)(F)F)cc1)c1ccc(Cl)c(Cl)c1] | tert-butyl (2-(3,4-dichlorophenyl)- 2-((4- (trifluoromethoxy)phenyl)sulfonamido) ethyl)carbamate | 15.92 |
| 225 | 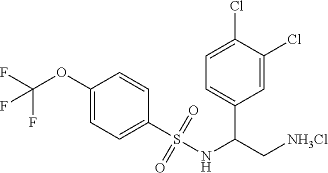 | N-(2-amino-1-(3,4- dichlorophenyl)ethyl)-4- (trifluoromethoxy)benzenesulfonamide hydrochloride | 21.76 |
| 226 | 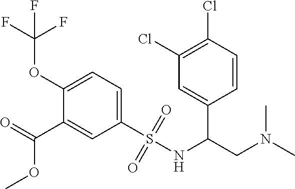 [SMILES: COC(=O)c1cc(S(=O)(=O)NC(CN(C)C)c2ccc(Cl)c(Cl)c2)ccc1OC(F)(F)F] | methyl 5-(N-(1-(3,4- dichlorophenyl)-2- (dimethylamino)ethyl)sulfamoyl)- 2-(trifluoromethoxy)benzoate | 70.57 |
| 227 | 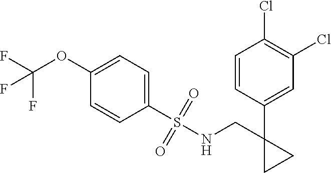 [SMILES: O=S(=O)(NCC1(c2ccc(Cl)c(Cl)c2)CC1)c1ccc(OC(F)(F)F)cc1] | N-((1-(3,4- dichlorophenyl)cyclopropyl) methyl)-4- (trifluoromethoxy)benzenesulfonamide | 48.58 |
| 228 | 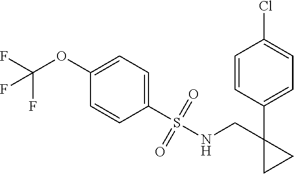 [SMILES: O=S(=O)(NCC1(c2ccc(Cl)cc2)CC1)c1ccc(OC(F)(F)F)cc1] | N-((1-(4- chlorophenyl)cyclopropyl)methyl)-4- (trifluoromethoxy)benzenesulfonamide | 59.88 |
| 229 | 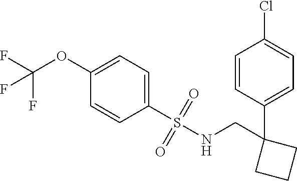 [SMILES: O=S(=O)(NCC1(c2ccc(Cl)cc2)CCC1)c1ccc(OC(F)(F)F)cc1] | N-((1-(4- chlorophenyl)cyclobutyl)methyl)-4- (trifluoromethoxy)benzenesulfonamide | 42.46 |
| 230 | 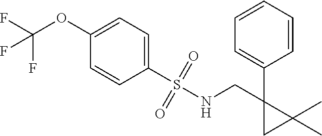 [SMILES: CC1(C)CC1(CNS(=O)(=O)c1ccc(OC(F)(F)F)cc1)c1ccccc1] | N-((2,2-dimethyl-1- phenylcyclopropyl)methyl)-4- (trifluoromethoxy)benzenesulfonamide | 37.33 |
| 231 | 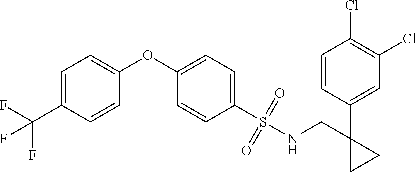 [SMILES: O=S(=O)(NCC1(c2ccc(Cl)c(Cl)c2)CC1)c1ccc(Oc2ccc(C(F)(F)F)cc2)cc1] | N-((1-(3,4- dichlorophenyl)cyclopropyl)methyl)-4- (4-(trifluoromethyl)phenoxy) benzenesulfonamide | 47.7 |
| 232 | 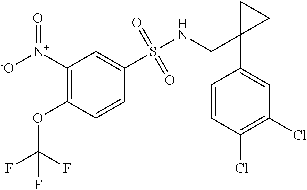 | N-((1-(3,4- dichlorophenyl)cyclopropyl) methyl)-3-nitro-4- (trifluoromethoxy)benzenesulfonamide | 31.08 |
| 233 | 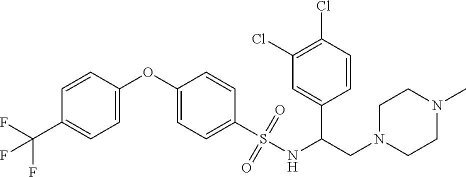 [SMILES: CN1CCN(CC(NS(=O)(=O)c2ccc(Oc3ccc(C(F)(F)F)cc3)cc2)c2ccc(Cl)c(Cl)c2)CC1] | N-(1-(3,4-dichlorophenyl)-2-(4- methylpiperazin-1-yl)ethyl)-4-(4- (trifluoromethyl)phenoxy) benzenesulfonamide | 6.193 |
| 234 | 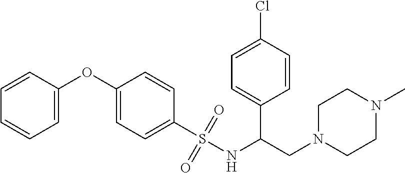 [SMILES: CN1CCN(CC(NS(=O)(=O)c2ccc(Oc3ccccc3)cc2)c2ccc(Cl)cc2)CC1] | N-(1-(4-chlorophenyl)-2-(4- methylpiperazin-1-yl)ethyl)-4- phenoxybenzenesulfonamide | 12.13 |
| 235 | 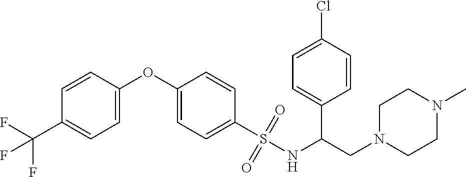 [SMILES: CN1CCN(CC(NS(=O)(=O)c2ccc(Oc3ccc(C(F)(F)F)cc3)cc2)c2ccc(Cl)cc2)CC1] | N-(1-(4-chlorophenyl)-2-(4- methylpiperazin-1-yl)ethyl)-4-(4- (trifluoromethyl)phenoxy) benzenesulfonamide | 11.28 |
| 236 | 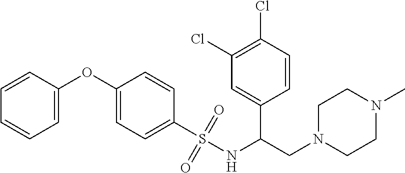 [SMILES: CN1CCN(CC(NS(=O)(=O)c2ccc(Oc3ccccc3)cc2)c2ccc(Cl)c(Cl)c2)CC1] | N-(1-(3,4-dichlorophenyl)-2-(4- methylpiperazin-1-yl)ethyl)-4- phenoxybenzenesulfonamide | 12.33 |
| 237 | 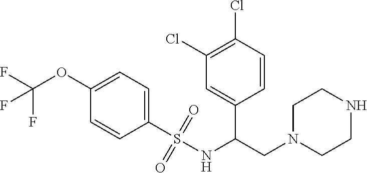 [SMILES: O=S(=O)(NC(CN1CCNCC1)c1ccc(Cl)c(Cl)c1)c1ccc(OC(F)(F)F)cc1] | N-(1-(3,4-dichlorophenyl)-2- (piperazin-1-yl)ethyl)-4- (trifluoromethoxy)benzenesulfonamide | 6.84 |
| 238 | 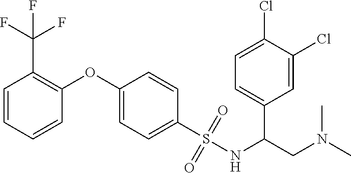 [SMILES: CN(C)CC(NS(=O)(=O)c1ccc(Oc2ccccc2C(F)(F)F)cc1)c1ccc(Cl)c(Cl)c1] | N-(1-(3,4-dichlorophenyl)-2- (dimethylamino)ethyl)-4-(2- (trifluoromethyl)phenoxy) benzenesulfonamide | 21.92 |
| 239 | 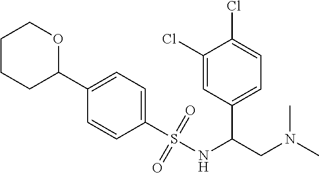 [SMILES: CN(C)CC(NS(=O)(=O)c1ccc(C2CCCCO2)cc1)c1ccc(Cl)c(Cl)c1] | N-(1-(3,4-dichlorophenyl)-2- (dimethylamino)ethyl)-4- (tetrahydro-2H-pyran-2- yl)benzenesulfonamide | 28.22 |
| 240 | 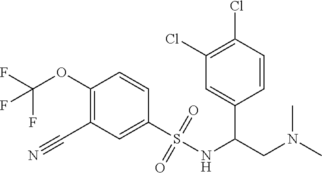 [SMILES: CN(C)CC(NS(=O)(=O)c1ccc(OC(F)(F)F)c(C#N)c1)c1ccc(Cl)c(Cl)c1] | 3-cyano-N-(1-(3,4- dichlorophenyl)-2- (dimethylamino)ethyl)-4- (trifluoromethoxy)benzenesulfonamide | 24.58 |
| 241 | 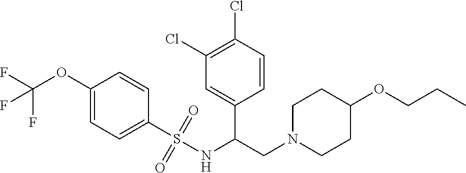 [SMILES: CCCOC1CCN(CC(NS(=O)(=O)c2ccc(OC(F)(F)F)cc2)c2ccc(Cl)c(Cl)c2)CC1] | N-(1-(3,4-dichlorophenyl)-2-(4- propoxypiperidin-1-yl)ethyl)-4- (trifluoromethoxy)benzenesulfonamide | 31.95 |
| 242 | 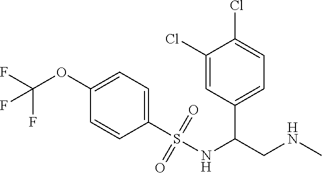 [SMILES: CNCC(NS(=O)(=O)c1ccc(OC(F)(F)F)cc1)c1ccc(Cl)c(Cl)c1] | N-(1-(3,4-dichlorophenyl)-2- (methylamino)ethyl)-4- (trifluoromethoxy)benzenesulfonamide | 24.06 |
| 243 | 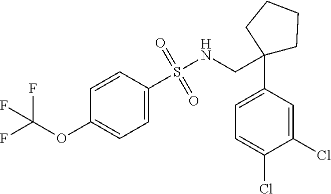 [SMILES: O=S(=O)(NCC1(c2ccc(Cl)c(Cl)c2)CCCC1)c1ccc(OC(F)(F)F)cc1] | N-((1-(3,4- dichlorophenyl)cyclopentyl)methyl)-4- (trifluoromethoxy)benzenesulfonamide | 51.67 |
| 244 | 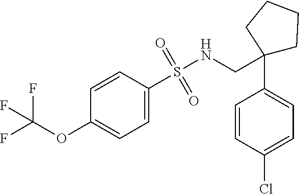 [SMILES: O=S(=O)(NCC1(c2ccc(Cl)cc2)CCCC1)c1ccc(OC(F)(F)F)cc1] | N-((1-(4- chlorophenyl)cyclopentyl)methyl)-4- (trifluoromethoxy)benzenesulfonamide | 56.11 |
| 245 | 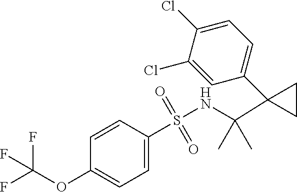 [SMILES: CC(C)(NS(=O)(=O)c1ccc(OC(F)(F)F)cc1)C1(c2ccc(Cl)c(Cl)c2)CC1] | N-(2-(1-(3,4- dichlorophenyl)cyclopropyl) propan-2-yl)-4- (trifluoromethoxy)benzenesulfonamide | 45.17 |
| 246 | 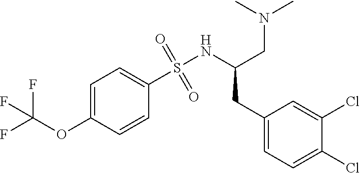 [SMILES: CN(C)C[C@@H](Cc1ccc(Cl)c(Cl)c1)NS(=O)(=O)c1ccc(OC(F)(F)F)cc1] | (R)-N-(1-(3,4-dichlorophenyl)-3- (dimethylamino)propan-2-yl)-4- (trifluoromethoxy)benzenesulfonamide | 23.83 |
| 247 | 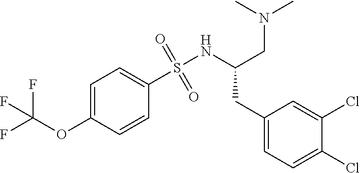 [SMILES: CN(C)C[C@H](Cc1ccc(Cl)c(Cl)c1)NS(=O)(=O)c1ccc(OC(F)(F)F)cc1] | (S)-N-(1-(3,4-dichlorophenyl)-3- (dimethylamino)propan-2-yl)-4- (trifluoromethoxy)benzenesulfonamide | 22.82 |
| 248 | 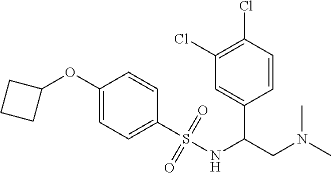 [SMILES: CN(C)CC(NS(=O)(=O)c1ccc(OC2CCC2)cc1)c1ccc(Cl)c(Cl)c1] | 4-cyclobutoxy-N-(1-(3,4- dichlorophenyl)-2- (dimethylamino)ethyl) benzenesulfonamide | 16.42 |
| 249 | 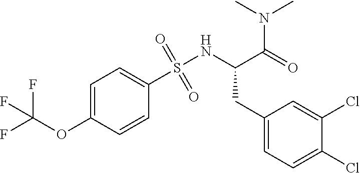 [SMILES: CN(C)C(=O)[C@H](Cc1ccc(Cl)c(Cl)c1)NS(=O)(=O)c1ccc(OC(F)(F)F)cc1] | (S)-3-(3,4-dichlorophenyl)-N,N- dimethyl-2-((4- (trifluoromethoxy)phenyl) sulfonamido)propanamide | 65.27 |
| 250 | 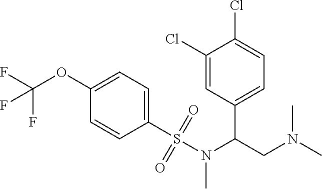 [SMILES: CN(C)CC(c1ccc(Cl)c(Cl)c1)N(C)S(=O)(=O)c1ccc(OC(F)(F)F)cc1] | N-(1-(3,4-dichlorophenyl)-2- (dimethylamino)ethyl)-N-methyl- 4- (trifluoromethoxy)benzenesulfonamide | 39.65 |
| 251 | 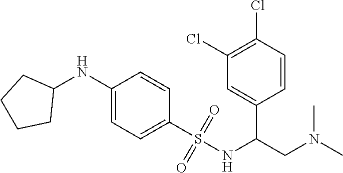 [SMILES: CN(C)CC(NS(=O)(=O)c1ccc(NC2CCCC2)cc1)c1ccc(Cl)c(Cl)c1] | 4-(cyclopentylamino)-N-(1-(3,4- dichlorophenyl)-2- (dimethylamino)ethyl) benzenesulfonamide | 22.4 |
| 252 | 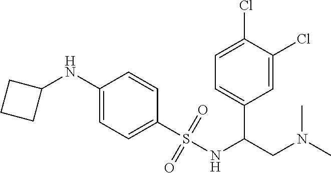 [SMILES: CN(C)CC(NS(=O)(=O)c1ccc(NC2CCC2)cc1)c1ccc(Cl)c(Cl)c1] | 4-(cyclobutylamino)-N-(1-(3,4- dichlorophenyl)-2- (dimethylamino)ethyl) benzenesulfonamide | 22.76 |
| 253 | 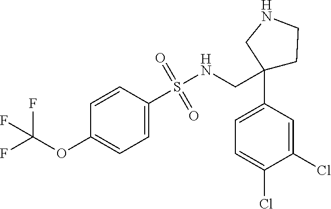 [SMILES: O=S(=O)(NCC1(c2ccc(Cl)c(Cl)c2)CCNC1)c1ccc(OC(F)(F)F)cc1] | N-((3-(3,4- dichlorophenyl)pyrrolidin-3- yl)methyl)-4- (trifluoromethoxy)benzenesulfonamide | 10.71 |
| 254 | 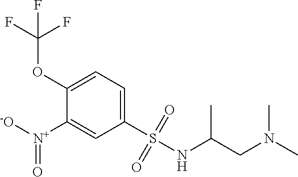 | N-(1-(dimethylamino)propan-2- yl)-3-nitro-4- (trifluoromethoxy)benzenesulfonamide | 1.92 |
| 255 | 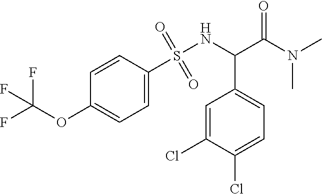 [SMILES: CN(C)C(=O)C(NS(=O)(=O)c1ccc(OC(F)(F)F)cc1)c1ccc(Cl)c(Cl)c1] | 2-(3,4-dichlorophenyl)-N,N- dimethyl-2-((4- (trifluoromethoxy)phenyl) sulfonamido)acetamide | 38.49 |
| 256 | 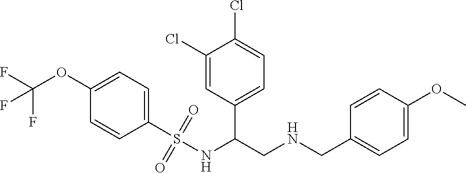 [SMILES: COc1ccc(CNCC(NS(=O)(=O)c2ccc(OC(F)(F)F)cc2)c2ccc(Cl)c(Cl)c2)cc1] | N-(1-(3,4-dichlorophenyl)-2-((4- methoxybenzyl)amino)ethyl)-4- (trifluoromethoxy)benzenesulfonamide | 34.05 |
| 257 | 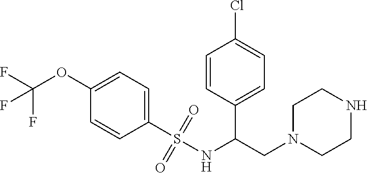 [SMILES: O=S(=O)(NC(CN1CCNCC1)c1ccc(Cl)cc1)c1ccc(OC(F)(F)F)cc1] | N-(1-(4-chlorophenyl)-2- (piperazin-1-yl)ethyl)-4- (trifluoromethoxy)benzenesulfonamide | 11.62 |
| 258 | 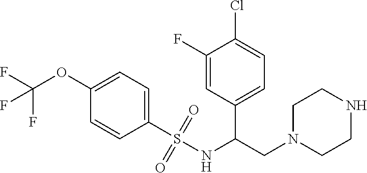 [SMILES: O=S(=O)(NC(CN1CCNCC1)c1ccc(Cl)c(F)c1)c1ccc(OC(F)(F)F)cc1] | N-(1-(4-chloro-3-fluorophenyl)- 2-(piperazin-1-yl)ethyl)-4- (trifluoromethoxy)benzenesulfonamide | 11.33 |
| 259 | 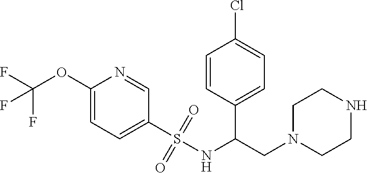 [SMILES: O=S(=O)(NC(CN1CCNCC1)c1ccc(Cl)cc1)c1ccc(OC(F)(F)F)nc1] | N-(1-(4-chlorophenyl)-2- (piperazin-1-yl)ethyl)-6- (trifluoromethoxy)pyridine-3- sulfonamide | 48.84 |
| 260 | 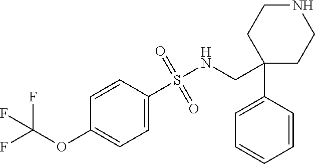 [SMILES: O=S(=O)(NCC1(c2ccccc2)CCNCC1)c1ccc(OC(F)(F)F)cc1] | N-((4-phenylpiperidin-4- yl)methyl)-4- (trifluoromethoxy)benzenesulfonamide | 33.56 |
| 261a | 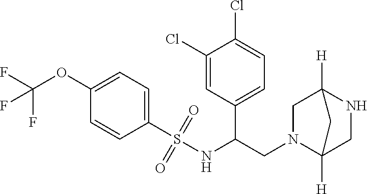 [SMILES: O=S(=O)(NC(CN1CC2CC1CN2)c1ccc(Cl)c(Cl)c1)c1ccc(OC(F)(F)F)cc1] | N-(2-(2,5- diazabicyclo[2.2.1]heptan-2- yl)-1-(3,4- dichlorophenyl)ethyl)-4- (trifluoromethoxy)benzenesulfonamide diastereomer I (racemic) | 10.48 |
| 261b | 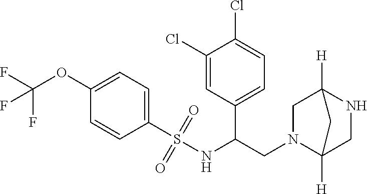 [SMILES: O=S(=O)(NC(CN1CC2CC1CN2)c1ccc(Cl)c(Cl)c1)c1ccc(OC(F)(F)F)cc1] | N-(2-(2,5- diazabicyclo[2.2.1]heptan-2- yl)-1-(3,4- dichlorophenyl)ethyl)-4- (trifluoromethoxy)benzenesulfonamide diastereomer II (racemic) | 9.79 |
| 262 | 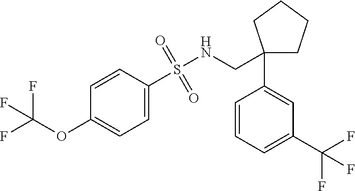 [SMILES: O=S(=O)(NCC1(c2cccc(C(F)(F)F)c2)CCCC1)c1ccc(OC(F)(F)F)cc1] | 4-(trifluoromethoxy)-N-((1-(3- (trifluoromethyl)phenyl) cyclopentyl)methyl)benzenesulfonamide | 63.1 |
| 263 | 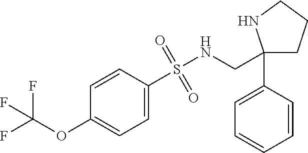 [SMILES: O=S(=O)(NCC1(c2ccccc2)CCCN1)c1ccc(OC(F)(F)F)cc1] | N-((2-phenylpyrrolidin-2- yl)methyl)-4- (trifluoromethoxy)benzenesulfonamide | 75.72^{a} |
| 264 | 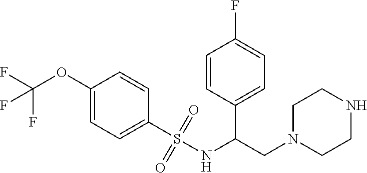 [SMILES: O=S(=O)(NC(CN1CCNCC1)c1ccc(F)cc1)c1ccc(OC(F)(F)F)cc1] | N-(1-(4-fluorophenyl)-2- (piperazin-1-yl)ethyl)-4- (trifluoromethoxy)benzenesulfonamide | 43.1 |
| 265 | 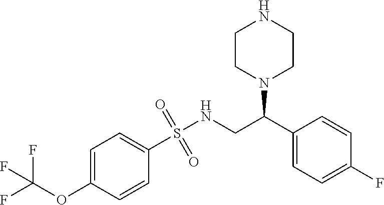 [SMILES: O=S(=O)(NC[C@H](c1ccc(F)cc1)N1CCNCC1)c1ccc(OC(F)(F)F)cc1] | (S)-N-(2-(4-fluorophenyl)-2- (piperazin-1-yl)ethyl)-4- (trifluoromethoxy)benzenesulfonamide | 40.3 |
| 266 | 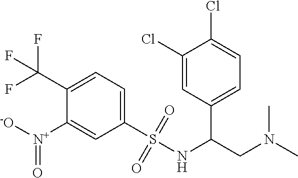 | N-(1-(3,4-dichlorophenyl)-2- (dimethylamino)ethyl)-3-nitro- 4- (trifluoromethyl)benzenesulfonamide | 41.85 |
| 267 | 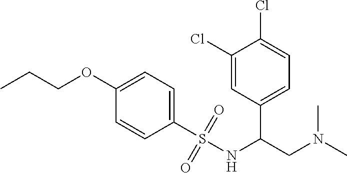 [SMILES: CCCOc1ccc(S(=O)(=O)NC(CN(C)C)c2ccc(Cl)c(Cl)c2)cc1] | N-(1-(3,4-dichlorophenyl)-2- (dimethylamino)ethyl)-4- propoxybenzenesulfonamide | 23.76 |
| 268 | 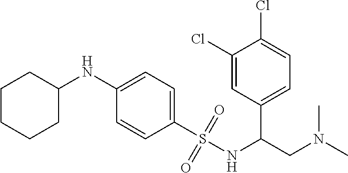 [SMILES: CN(C)CC(NS(=O)(=O)c1ccc(NC2CCCCC2)cc1)c1ccc(Cl)c(Cl)c1] | 4-(cyclohexylamino)-N-(1-(3,4- dichlorophenyl)-2- (dimethylamino)ethyl) benzenesulfonamide | 15.52 |
| 269 | 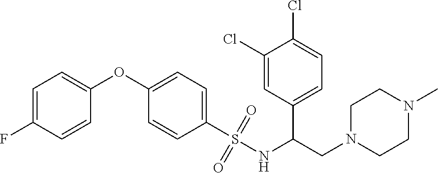 [SMILES: CN1CCN(CC(NS(=O)(=O)c2ccc(Oc3ccc(F)cc3)cc2)c2ccc(Cl)c(Cl)c2)CC1] | N-(1-(3,4-dichlorophenyl)-2-(4- methylpiperazin-1-yl)ethyl)-4- (4-fluorophenoxy) benzenesulfonamide | 15.04 |

^{a}GI₅₀ value corresponds to HCC1937 cell line assay

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

### Enumerated Embodiments

The following exemplary embodiments are provided, the numbering of which is not to be construed as designating levels of importance:

Embodiment 1 provides a compound of formula (I), or a salt, solvate, enantiomer, diastereoisomer, isotopologue, or tautomer thereof:

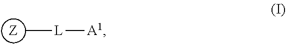

wherein:
A¹ is selected from the group consisting of

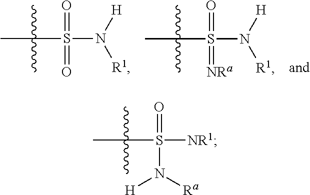

L is a linker comprising a bond, C₁-C₆ alkylene, or C₂-C₆ alkenylene;
ring Z is optionally substituted phenyl, optionally substituted naphthyl, optionally substituted pyridyl, or optionally substituted cyclohexyl,
   wherein each optional substituent is at least one substituent selected from the group consisting of C₁-C₆ alkyl, C₁-C₆ haloalkyl, C₁-C₆ aminoalkyl, C₁-C₆ hydroxyalkyl, C₂-C₆ alkenyl, C₂-C₆ alkynyl, C₁-C₆ alkoxy, C₁-C₆ haloalkoxy, C₆-C₁₀ aryl, C₄-C₁₀ heteroaryl, C₆-C₁₀ aryloxy, C₄-C₁₀ heteroaryloxy F, Cl, Br, I, OH, CN, N(R^{a})(R^{b}), NO₂, C(═O)OR^{a}, and C(═O)N(R^{a})(R^{b}),
   wherein each optional C₆-C₁₀ aryl, C₄-C₁₀ heteroaryl, C₄-C₁₀ heteroaryloxy, or C₆-C₁₀ aryloxy substituent is independently optionally substituted with at least one substituent selected from the group consisting of C₁-C₃ alkyl, C₁-C₃ haloalkyl, C₁-C₃ alkoxy, C₁-C₃ haloalkoxy, F, Cl, Br, I, OH, CN, N(R^{a})(R^{b}), NO₂, OH, C(═O)OR^{a}, and C(═O)N(R^{a})(R^{b}),
   wherein two adjacent substituents of ring Z may optionally combine to provide a 5 to 8-membered fused ring;
R¹ is

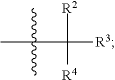

R² is selected from the group consisting of optionally substituted C₁-C₆ alkyl, optionally substituted C₁-C₆ haloalkyl, optionally substituted C₁-C₆ aminoalkyl, optionally substituted C₁-C₆ hydroxyalkyl, optionally substituted C₂-C₆ alkenyl, optionally substituted C₅-C₈ cycloalkenyl, optionally substituted C₂-C₆ alkynyl, optionally substituted C₁-C₆ alkoxy, optionally substituted C₁-C₆ haloalkoxy, optionally substituted C₃-C₁₂ cycloalkyl, optionally substituted C₂-C₁₂ heterocycloalkyl, optionally substituted C₆-C₁₀ aryl, and optionally substituted C₄-C₁₀ heteroaryl,
   wherein each optional substituent in R² is independently selected from the group consisting of C₁-C₃ alkyl, C₁-C₃ haloalkyl, C₁-C₃ alkoxy, C₃-C₅ cycloalkoxy, C₃-C₁₂ heterocycloalkyl, C₁-C₃ haloalkoxy, F, Cl, Br, OR, CN, NO₂, C(═O)OR^{a}, C(═O)N(R^{a})₂, S(═O)₂R^{b}, NR^{a}S(═O)₂R^{b}, C₄-C₁₀ heteroaryl, benzyl, and phenyl, wherein the benzyl and/or phenyl are each independently optionally substituted with at least one substituent selected from the group consisting of C₁-C₆ alkyl, C₁-C₆ alkoxy, C₁-C₃ haloalkyl, C₁-C₃ haloalkoxy, and halogen;
R³ is selected from the group consisting of H, C(═O)R^{a}, C(═O)OR^{a}, C(═O)N(R^{a})₂, NR^{a}C(═O)OR^{b}, optionally substituted C₁-C₆ alkyl, optionally substituted C₁-C₆ alkoxy, optionally substituted C₁-C₆ haloalkyl, optionally substituted C₁-C₆ aminoalkyl, optionally substituted C₁-C₆ hydroxyalkyl, optionally substituted C₃-C₁₂ cycloalkyl, and optionally substituted C₂-C₁₂ heterocycloalkyl,
   wherein each optional substituent in R³ is selected from the group consisting of C₁-C₃ alkyl, C₂-C₁₂ heterocycloalkyl, C₁-C₃ haloalkyl, C₁-C₃ alkoxy, C₁-C₃ haloalkoxy, benzyl, C₆-C₁₀ aryl, C₄-C₁₀ heteroaryl, F, Cl, Br, OH, CN, NO₂, C(═O)OR^{a}, C(═O)N(R^{a})(R^{b}), NR^{a}C(═O)OR^{a}, S(═O)₂R^{b}, and NR^{a}S(═O)₂R^{b},
R⁴ is H or C₁-C₆ alkyl;
each occurrence of R^{a} is independently selected from the group consisting of H, C₁-C₆ alkyl, C₃-C₆ cycloalkyl, benzyl, and C₆-C₁₀ aryl;
each occurrence of R^{b} is independently selected from the group consisting of H, optionally substituted C₁-C₆ alkyl, optionally substituted C₂-C₁₂ heterocycloalkyl, optionally substituted benzyl, optionally substituted C₂-C₅ heteroaryl, optionally substituted phenyl, and optionally substituted naphthyl,
   wherein each optional substituent in R^{b} is independently selected from the group consisting of C₁-C₃ alkyl, C₁-C₃ haloalkyl, C₁-C₃ alkoxy, C₁-C₃ haloalkoxy, F, Cl, Br, I, OH, CN, NO₂, C(═O)OR^{a}, and C(═O)N(R^{a})₂;
wherein no more than one of R² and R³ is CH₂OH;
wherein if R³ is H and R⁴ is H, R³ is CH₃ and R⁴ is H, or R³ is H and R⁴ is CH₃, then one of the following applies:
   (a) R² comprises at least one substituent selected from the group consisting of C₆-C₁₀ aryl and C₄-C₁₀ heteroaryl, wherein the aryl and/or heteroaryl are each independently substituted with at least one substituent selected from the group consisting of OR, N(R^{a})(R^{a}), C₁-C₆ alkyl, C₁-C₃ haloalkyl, C₁-C₃ haloalkoxy, C₂-C₁₂ heterocycloalkyl, F, Cl, Br, and S(═O)₂R^{b};
   (b) R² comprises at least one substituent selected from the group consisting of C₃-C₆ cycloalkyl and C₂-C₁₂ heterocycloalkyl, and at least one substituent selected from the group consisting of C₆-C₁₀ aryl and C₄-C₁₀ heteroaryl;
wherein if L is a bond, R³ is H, and R⁴ is H, and A¹ is

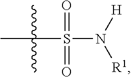

then the optionally substituted C₂-C₁₂ heterocycloalkyl in R² comprises no more than one phenyl substituted with at least one halogen;
wherein if Z is optionally substituted phenyl, L is a bond, A¹ is

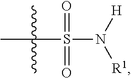

and R² is optionally substituted C₆-C₁₀ aryl, then one of the following applies:
(a) the phenyl in Z is substituted with at least one substituent selected from the group consisting of C₁-C₆ haloalkyl, C₁-C₆ aminoalkyl, C₁-C₆ hydroxyalkyl, C₂-C₆ alkenyl, C₂-C₆ alkynyl, C₁-C₆ alkoxy, C₁-C₆ haloalkoxy, C₆-C₁₀ aryl, C₃-C₆ heterocycloalkyl, C₄-C₁₀ heteroaryl, C₆-C₁₀ aryloxy, C₄-C₁₀ heteroaryloxy F, Cl, Br, I, OH, CN, N(R^{a})(R^{b}), NO₂, C(═O)OR^{a}, and C(═O)N(R^{a})(R^{b}), and the C₆-C₁₀ aryl in R² is substituted with at least one substituent selected from the group consisting of C₁-C₃ haloalkyl, C₁-C₃ alkoxy, C₃-C₅ cycloalkoxy, C₃-C₁₂ heterocycloalkyl, C₁-C₃ haloalkoxy, F, Cl, Br, OR^{b}, CN, NO₂, C(═O)OR^{a}, C(═O)N(R^{a})₂, S(═O)₂R^{b}, NR^{a}S(═O)₂R^{b}, C₄-C₁₀ heteroaryl, and benzyl;
(b) the phenyl in Z is substituted with at least one substituent, and the C₆-C₁₀ aryl in R² is substituted with at least two substituents; or
(c) the phenyl in Z is substituted with at least one trifluoromethoxy substituent, the C₆-C₁₀ aryl in R² is substituted with at least one substituent, and R³ is optionally substituted C₁-C₆ aminoalkyl;
wherein if Z is 4-trifluoromethoxyphenyl, L is a bond, and A¹ is

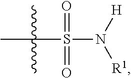

then each of the following apply:
(a) if R³ and R⁴ are each independently H, then R² is not selected from the group consisting of phenyl, 2-difluoromethoxyphenyl, 2-methylphenyl, 2-cyanophenyl, 2-methoxyphenyl, 2-trifluoromethoxyphenyl, 2-chlorophenyl, 2-fluorophenyl, 2-trifluoromethylphenyl, 3-methylphenyl, 3-methoxyphenyl, 3-bromophenyl, 3-chlorophenyl, 3-trifluoromethoxyphenyl, 3-trifluoromethylphenyl, 4-isobutyramido-phenyl, 4-tert-butylphenyl, 4-methoxyphenyl, 4-chlorophenyl, 4-bromophenyl, 4-carboxyphenyl, 4-trifluoromethylphenyl, 4-methylphenyl, 4-fluorophenyl, 2-fluoro-3-trifluoromethylphenyl, 2,3-dimethylphenyl, 2,3-dichlorophenyl, 2-methyl-4-chlorophenyl, 2-chloro-4-fluorophenyl, 2,4-dimethoxyphenyl, 2-methoxy-4-(2-methoxy-5-fluorophenyl)phenyl, 2-methoxy-4-chlorophenyl, 2-fluoro-4-trifluoromethylphenyl, 2,6-difluorophenyl, 2-chloro-6-methylphenyl, 2,6-dimethoxyphenyl, 3,4-ethylenedioxyphenyl, 3-fluoro-4-trifluoromethylphenyl, 3,4-difluorophenyl, 3-fluoro-4-methylphenyl, 3-methoxy-4-ethoxyphenyl, 3,4-dimethoxyphenyl, 3-chloro-4-methylphenyl, 3-methyl-4-fluorophenyl, 3-chloro-4-fluorophenyl, 3,5-difluorophenyl, 3,5-dichlorophenyl, 3,5-dimethoxyphenyl, 3,5-dimethylphenyl, 3,6-dichlorophenyl, 3,6-difluorophenyl, 3,6-dimethoxyphenyl, 3-chloro-6-methylphenyl, 2,3,4-trifluorophenyl, 3,4,5-trifluorophenyl, 2,6-difluoro-3-chlorophenyl, 1-((5-ethanolyl)thiophen-2-yl)cyclopentyl, and 1-(thiophen-2-yl)cyclopentyl;
(b) if R³ is H and R⁴ is methyl or R³ is methyl and R⁴ is H, then R² is not selected from the group consisting of phenyl, naphthyl, 3-methoxyphenyl, 2,4-difluorophenyl, 3,4-methylenedioxyphenyl, 3,4-(1,2-ethylenedioxy)phenyl, 3-fluoro-4-methoxyphenyl, 3,6-dimethoxyphenyl, 2-methylphenyl, 2-methoxyphenyl, 4-methylphenyl, 4-bromophenyl, 4-fluorophenyl, 4-methoxyphenyl, 4-ethoxyphenyl, 4-(methanesulfonyl)phenyl, 4-(3-imidazolyl)phenyl, and 4-(2-(1,2,4-triazol)-yl)phenyl;
(c) if R³ is H and R⁴ is ethyl or R³ is selected from the group consisting of ethyl and cyclopropyl and R⁴ is H, then R² is not selected from the group consisting of phenyl and 4-methoxyphenyl;
(d) if R³ is selected from the group consisting of hydroxymethyl, cyano, 1-(1,2,4-triazol)-yl, and 1-(1,2,5-triazol)-yl, and R⁴ is H, then R² is not phenyl;
(e) if R³ is CH₂C(═O)OH and R⁴ is H, then R² is not selected from the group consisting of phenyl, 2-chlorophenyl, 4-methylphenyl, 4-fluorophenyl, 4-chlorophenyl, 4-tert-butylphenyl, 4-methoxyphenyl, and 3,4-dimethoxyphenyl;
(f) if R³ is CH₂C(═O)OCH₃ and R⁴ is H, then R² is not selected from the group consisting of cyclohexyl, phenyl, 4-chlorophenyl, 4-tert-butylphenyl, 4-methylphenyl, 4-bromophenyl, 4-fluorophenyl, and 3-bromophenyl;
(g) if R³ is CH₂C(═O)OCH₂CH₃ and R⁴ is H, then R² is not selected from the group consisting of phenyl, 2-chlorophenyl, 4-fluorophenyl, and 4-bromophenyl;
(h) if R³ is CH₂(C═O)OCH₂Ph and R⁴ is H, then R² is not 2-chlorophenyl;
(i) if R³ is CH₂(C═O)OCH(CH₃)₂ and R⁴ is H, then R² is not 2-nitrophenyl;
(j) if R³ is selected from the group consisting of C(═O)NH₂, C(═O)OCH₃, and C(═O)OCH₂CH₃ and R⁴ is H, then R² is not phenyl; and
wherein if Z is 4-difluoromethoxyphenyl, L is a bond, and A¹ is

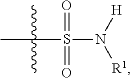

then each of the following apply:
(a) if R³ is CH₂(C═O)OH and R⁴ is H, then R² is not 2-chlorophenyl;
(b) if R³ is CH₂(C═O)OCH₃ and R⁴ is H, then R² is not selected from the group consisting of cyclohexyl, 4-chlorophenyl, and 4-bromophenyl;
(c) if R³ is CH₂(C═O)OCH₂CH₃ and R⁴ is H, then R² is not selected from the group consisting of phenyl, 2-chlorophenyl, and 4-bromophenyl; and
(d) if R³ is CH₂(C═O)OCH₂Ph and R⁴ is H, then R² is not 2-chlorophenyl.

Embodiment 2 provides the compound of Embodiment 1, wherein L is selected from the group consisting of —CH₂—, —CH₂CH₂—, and —CH═CH—.

Embodiment 3 provides the compound of Embodiment 1 or 2, wherein A¹ is selected from the group consisting of

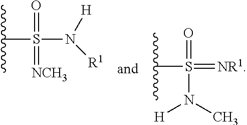

Embodiment 4 provides the compound of any one of Embodiments 1-3, wherein R¹ is selected from the group consisting of

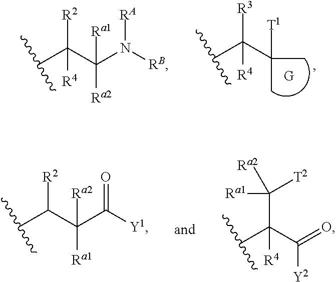

wherein:
R², if present, is selected from the group consisting of optionally substituted C₆-C₁₀ aryl and optionally substituted C₄-C₁₀ heteroaryl,
   wherein each optional substituent in R² is independently selected from the group consisting of C₁-C₃ alkyl, C₁-C₃ haloalkyl, C₁-C₃ alkoxy, C₃-C₅ cycloalkoxy, C₁-C₃ haloalkoxy, F, Cl, Br, OH, CN, NO₂, C(═O)OR^{a}, C(═O)N(R^{a})₂, S(═O)₂R^{b}, NR^{a}S(═O)₂R^{b}, C₄-C₁₀ heteroaryl, benzyl, and phenyl, wherein the benzyl and/or phenyl are each independently optionally substituted with at least one substituent selected from the group consisting of C₁-C₆ alkyl, C₁-C₆ alkoxy, C₁-C₃ haloalkyl, C₁-C₃ haloalkoxy, and halogen;
R³, if present, is selected from the group consisting of H and C₁-C₃ alkyl;
R⁴, if present, is selected from the group consisting of H and C₁-C₃ alkyl;
R^{A} and R^{B}, if present, are each independently selected from the group consisting of H, optionally substituted C₁-C₆ alkyl, optionally substituted benzyl, and C(═O)OR^{a},
   wherein each optional substituent in each of R^{A} and R^{B} is independently selected from the group consisting of C₁-C₆ alkyl and C₁-C₆ alkoxy; and
   wherein R^{A} and R^{B} may combine with the N to which they are bound to form a C₂-C₈ heterocycloalkyl;
R^{a1} and R^{a2}, if present, are each independently selected from the group consisting of H and C₁-C₃ alkyl;
T¹ and T², if present, are each independently phenyl optionally substituted with at least one selected from the group consisting of halogen, C₁-C₃ alkyl, C₁-C₃ haloalkyl, and C₁-C₃ haloalkoxy;
Ring G, if present, is selected from the group consisting of C₃-C₆ cycloalkyl and C₂-C₆ heterocycloalkyl, wherein ring G is optionally substituted with C₁-C₃ alkyl; and
Y¹ and Y², if present, are each independently selected from the group consisting of OR^{a} and N(R^{a})(R^{a}).

Embodiment 5 provides the compound of Embodiment 4, wherein R² is selected from the group consisting of:

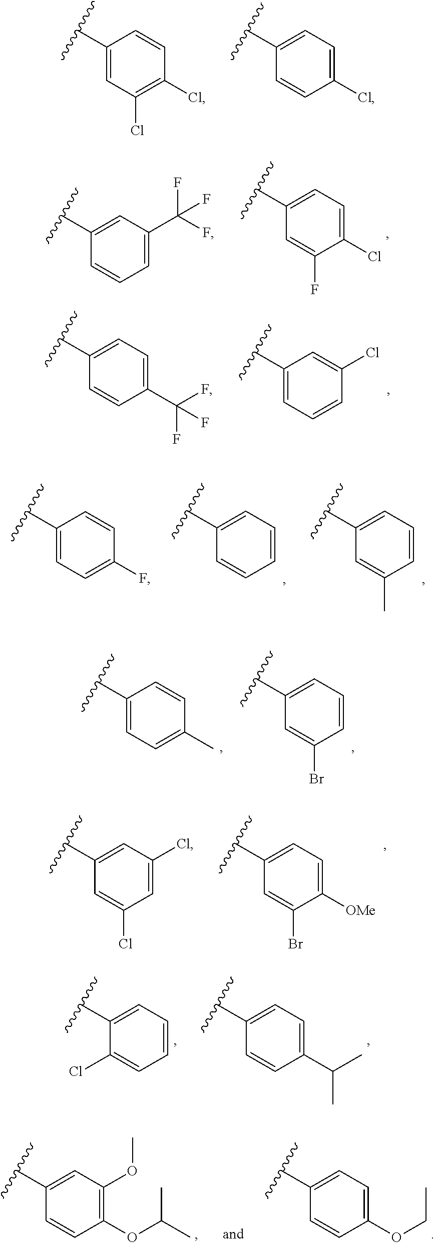

Embodiment 6 provides the compound of Embodiment 4, wherein R³ is selected from the group consisting of H and CH₃.

Embodiment 7 provides the compound of any one of Embodiments 4-6, wherein R⁴ is selected from the group consisting of H and CH₃.

Embodiment 8 provides the compound of any one of Embodiments 4-5 and 7, wherein at least one of R^{a1} and R^{a2} is H.

Embodiment 9 provides the compound of any one of Embodiments 4 and 6-7, wherein T¹ is selected from the group consisting of

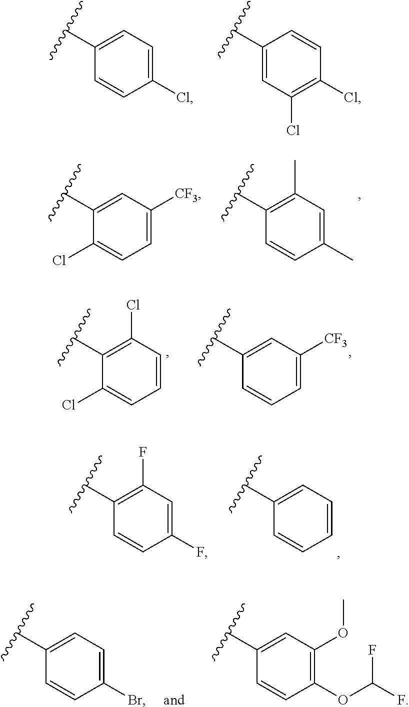

Embodiment 10 provides the compound of any one of Embodiments 4 and 7-8, wherein T² is selected from the group consisting of

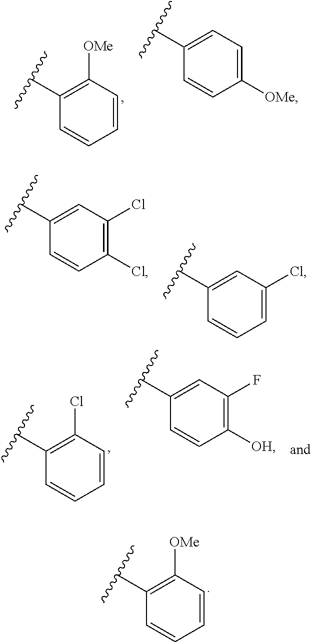

Embodiment 11 provides the compound of any one of Embodiments 4-5 and 8, wherein Y¹ is selected from the group consisting of OH, OCH₃, and OCH₂CH₃.

Embodiment 12 provides the compound of any one of Embodiments 4, 7-8 and 10, wherein Y² is selected from the group consisting of OCH₃ and OCH₂CH₃.

Embodiment 13 provides the compound of any one of Embodiments 4, 6-7 and 9, wherein ring G is selected from the group consisting of

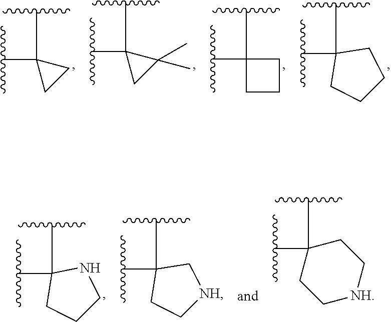

Embodiment 14 provides the compound of any one of Embodiments 4-5 and 7-8, wherein one of the following applies:
(a) R^{A} and R^{B} are each independently selected from the group consisting of H, CH₃, C(═O)O(C(CH₃)₃),

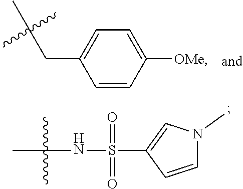

or
(b) R^{A} and R^{B} combine with the N atom to which they are bound to form one selected from the group consisting of

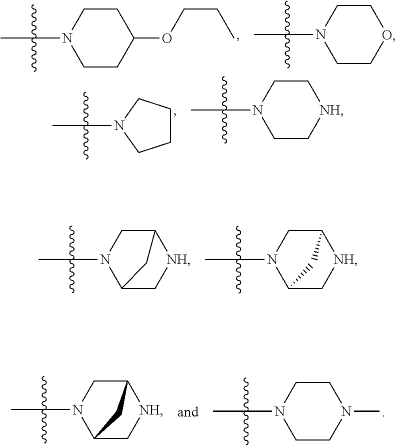

Embodiment 15 provides the compound of any one of Embodiments 1-14, wherein the compound of formula (I) is the compound of formula (II):

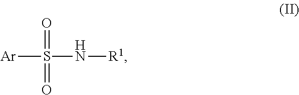

wherein:
Ar is phenyl, naphthyl, or pyridyl, any one of which is optionally substituted with at least one substituent selected from the group consisting of C₁-C₆ alkyl, C₁-C₆ haloalkyl, C₁-C₆ aminoalkyl, C₁-C₆ hydroxyalkyl, C₂-C₆ alkenyl, C₂-C₆ alkynyl, C₁-C₆ alkoxy, C₁-C₆ haloalkoxy, C₆-C₁₀ aryl, C₆-C₁₀ aryloxy, F, Cl, Br, I, OH, NH₂, CN, NO₂, C(═O)OR^{a}, and C(═O)N(R^{a})₂,
   wherein each C₆-C₁₀ aryl, C₄-C₁₀ heteroaryl, or C₆-C₁₀ aryloxy substituent is independently optionally substituted with at least one substituent selected from the group consisting of C₁-C₃ alkyl, C₁-C₃ haloalkyl, C₁-C₃ alkoxy, C₁-C₃ haloalkoxy, F, Cl, Br, I, OH, CN, NO₂, C(═O)OR^{a}, and C(═O)N(R^{a})(R^{a}),
   wherein two adjacent substituents of ring Z may optionally combine to provide a 4 to 8-membered fused ring.

Embodiment 16 provides the compound of Embodiment 15, wherein Ar is selected from the group consisting of:

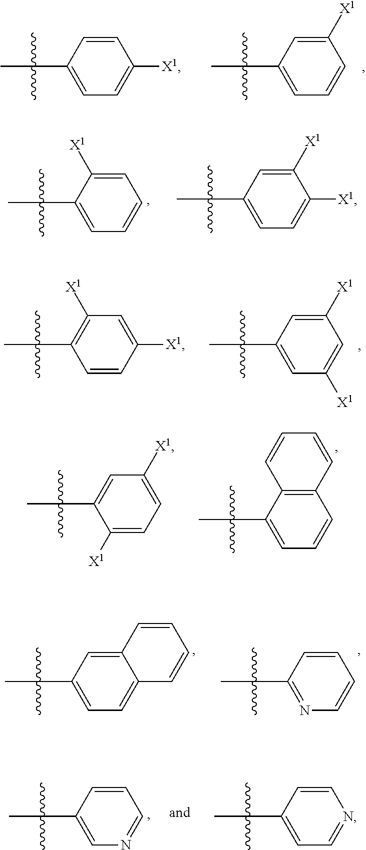

wherein each occurrence of X¹ is independently selected from the group consisting of methyl, ethyl, i-propyl, n-propyl, n-butyl, n-pentyl, Cl, F, OCH₂CH₃, OCH₂CH₂CH₃, OCH(CH₃)₂, CF₃, OCF₃, OCHF₂, CN, NO₂, C(═O)OR^{a}, C(═O)NHR^{a}, NH₂

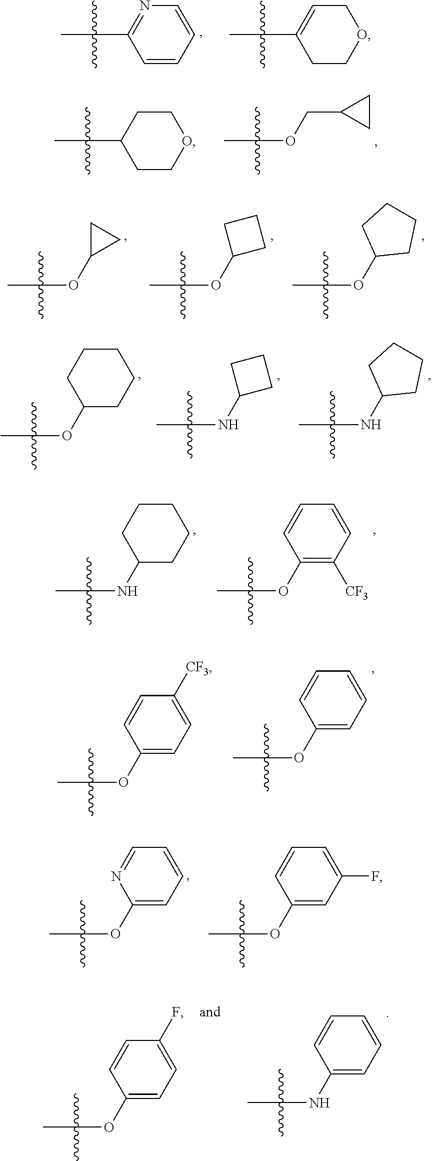

Embodiment 17 provides the compound of Embodiment 15 or 16, wherein Ar is selected from the group consisting of:

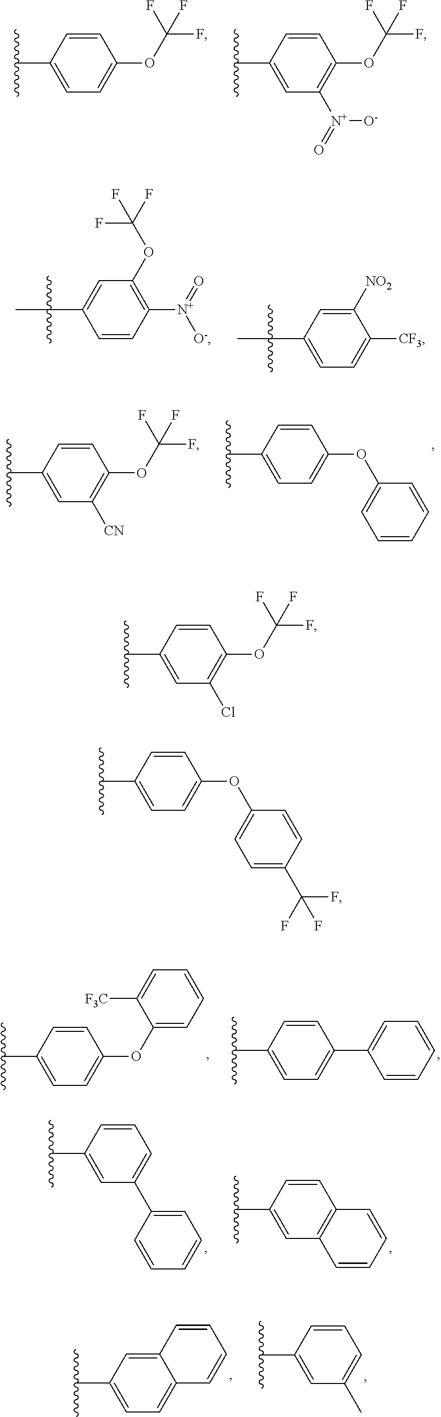

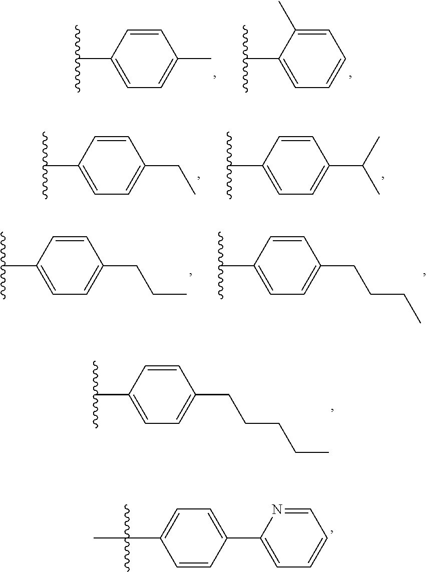

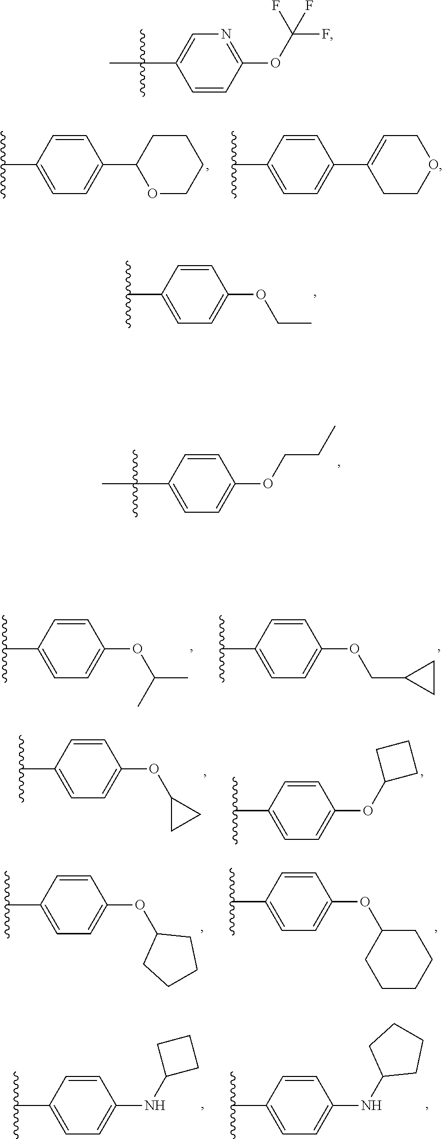

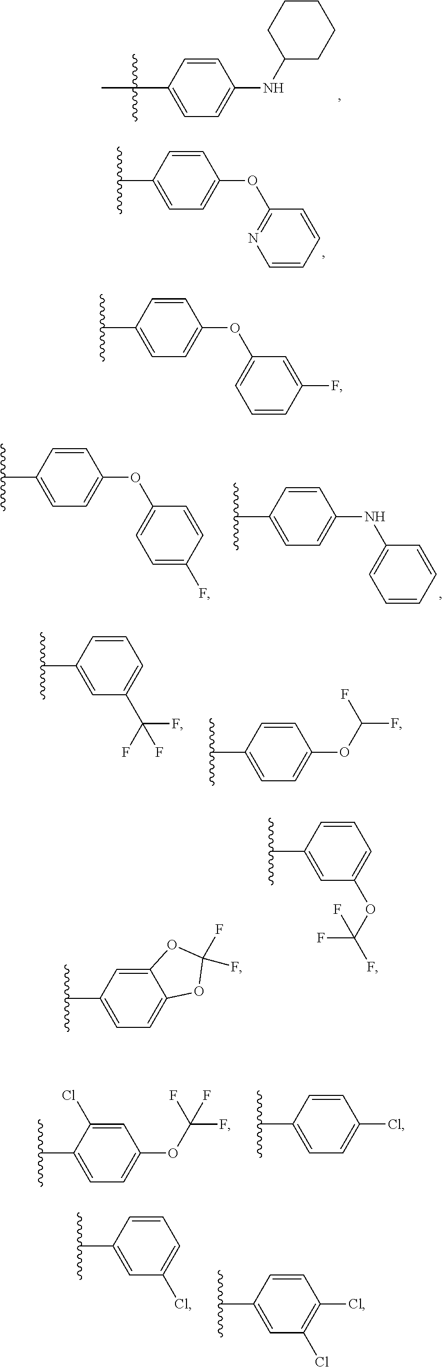

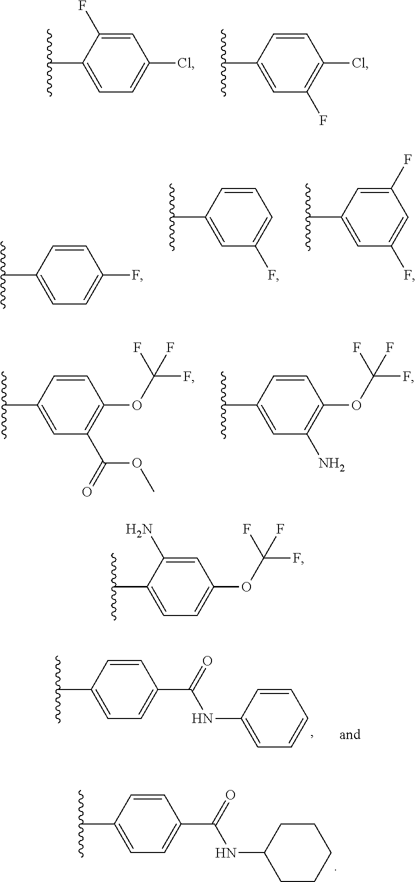

Embodiment 18 provides the compound of any one of Embodiments 15-17, wherein R² is selected from the group consisting of:

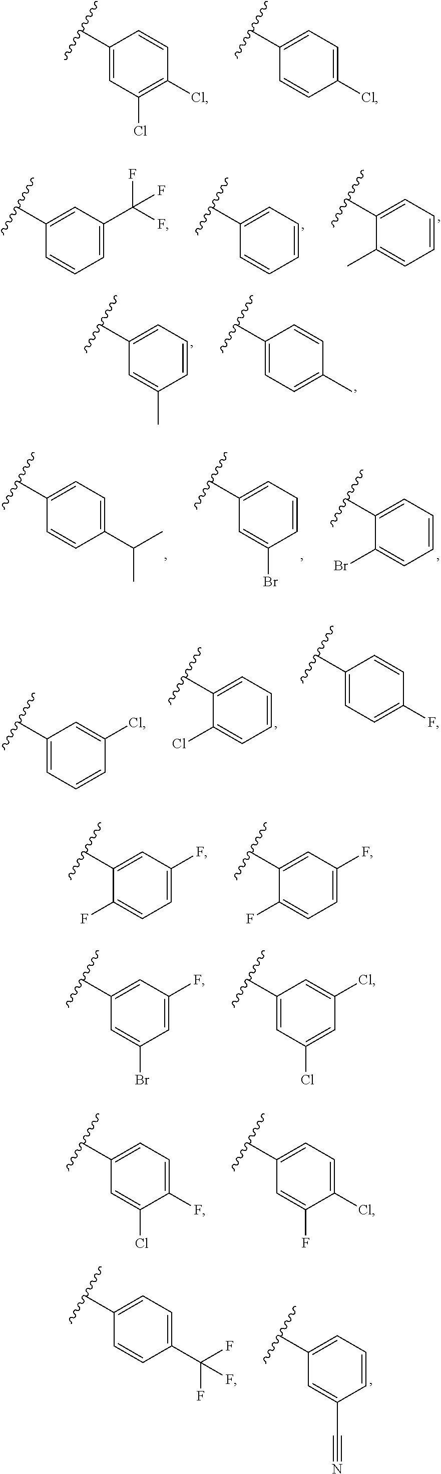

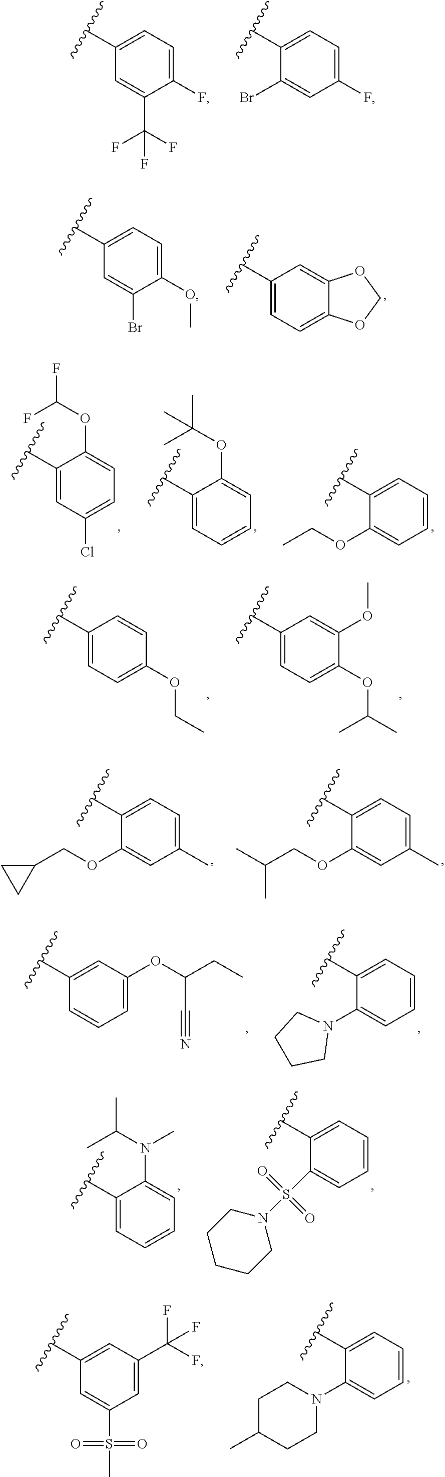

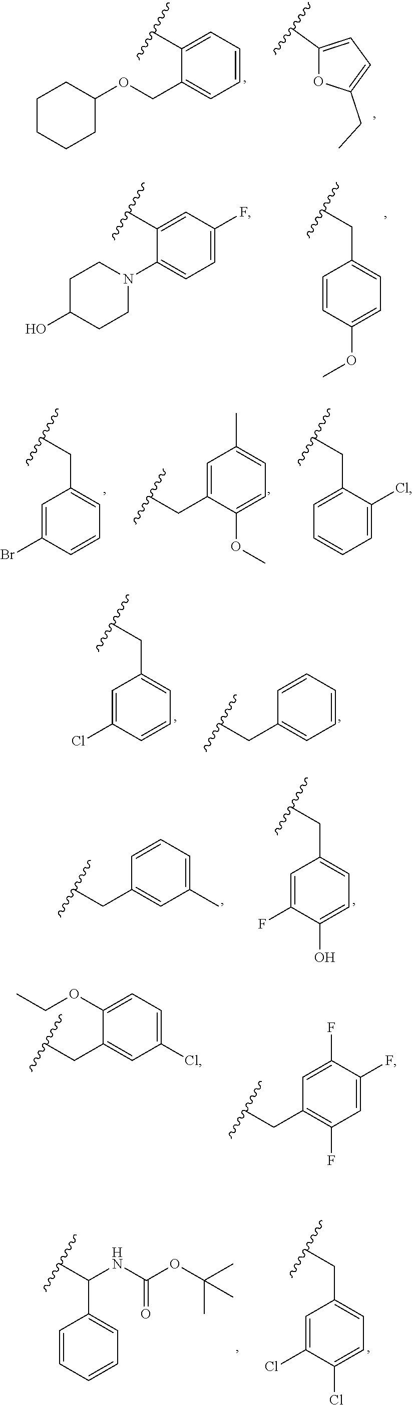

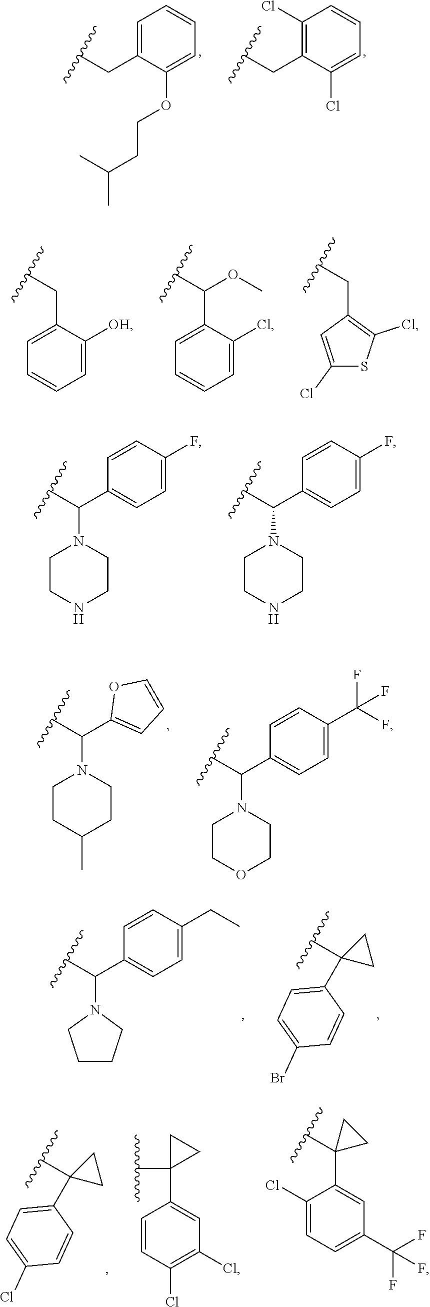

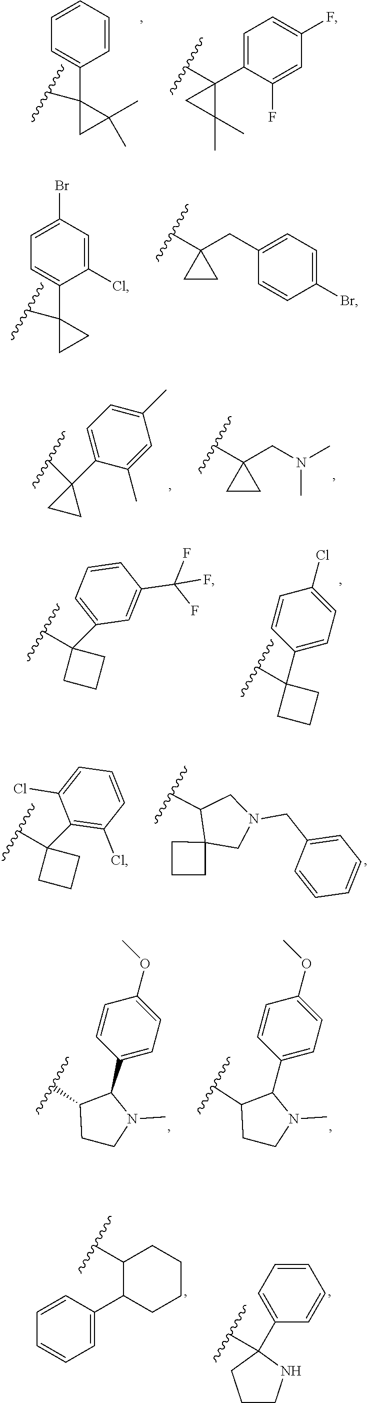

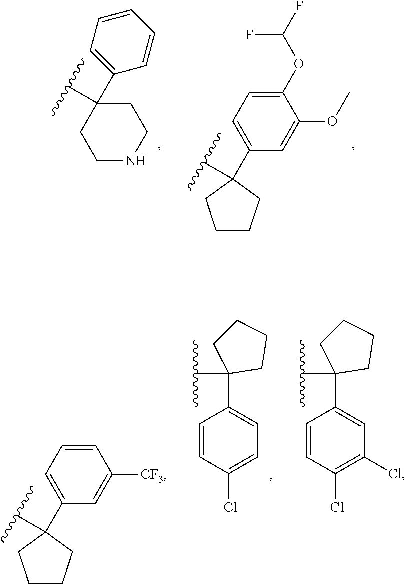

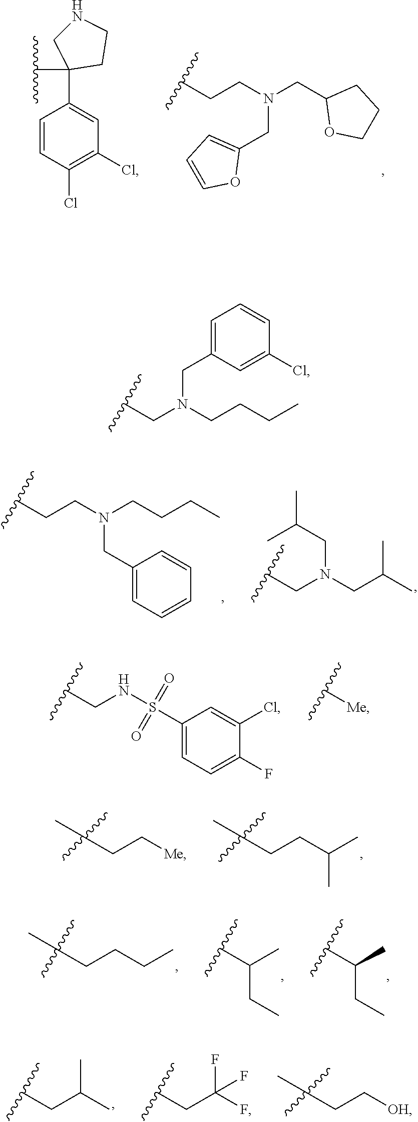

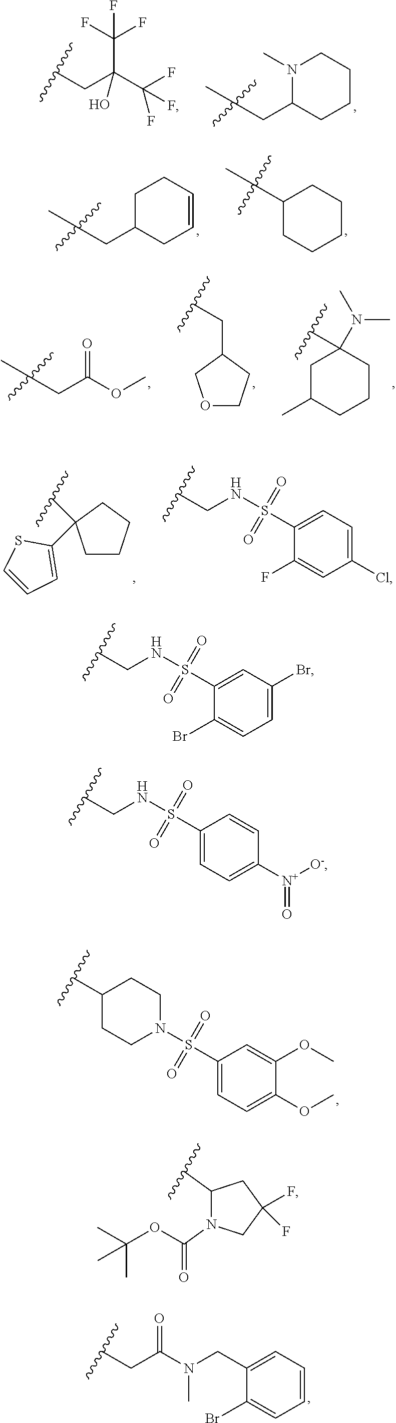

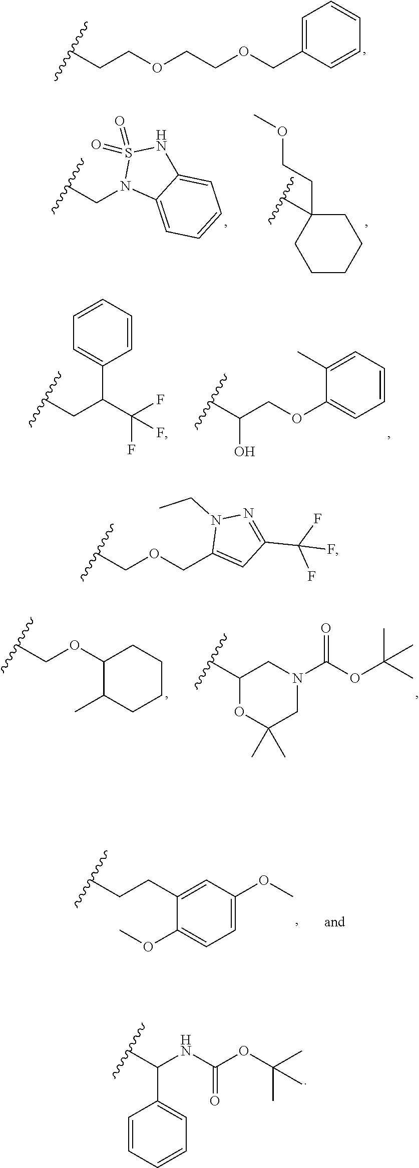

Embodiment 19 provides the compound of any one of Embodiments 15-18, wherein R³ is selected from the group consisting of:
methyl, ethyl, n-propyl, i-propyl, t-butyl,

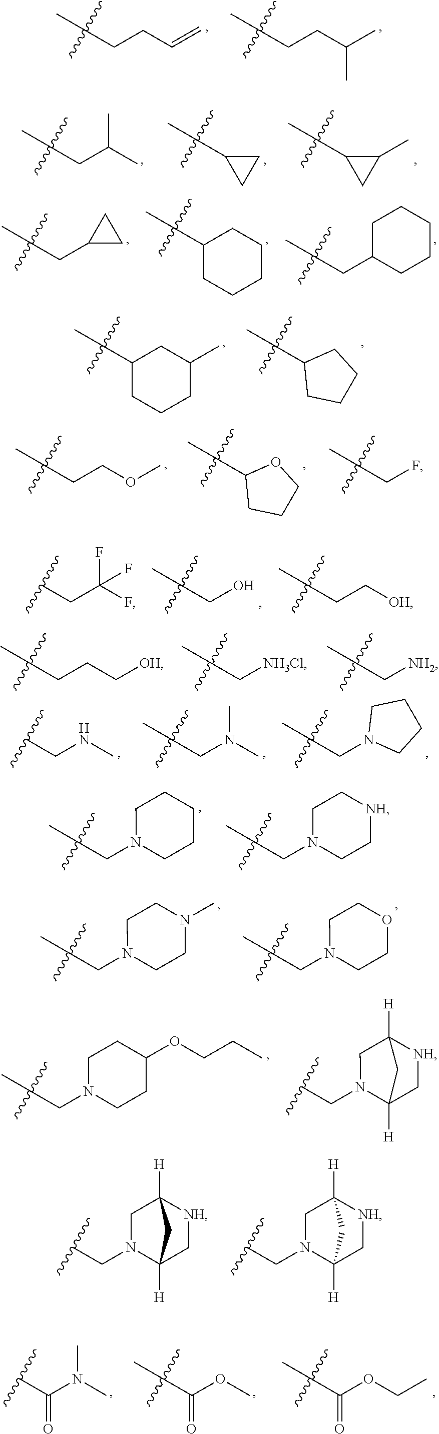

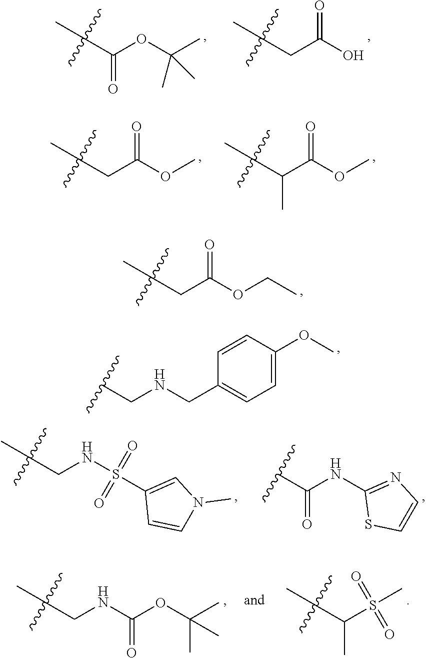

Embodiment 20 provides the compound of any one of Embodiments 15-19, wherein R⁴ is selected from the group consisting of methyl, ethyl, n-propyl, and

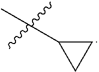 [SMILES: *C1CC1]

Embodiment 21 provides the compound of any one of Embodiments 15-17, wherein the compound of formula (II) is the compound of formula (IIa):

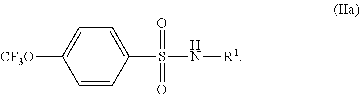

Embodiment 22 provides the compound of Embodiment 21, wherein R¹ is selected from the group consisting of:

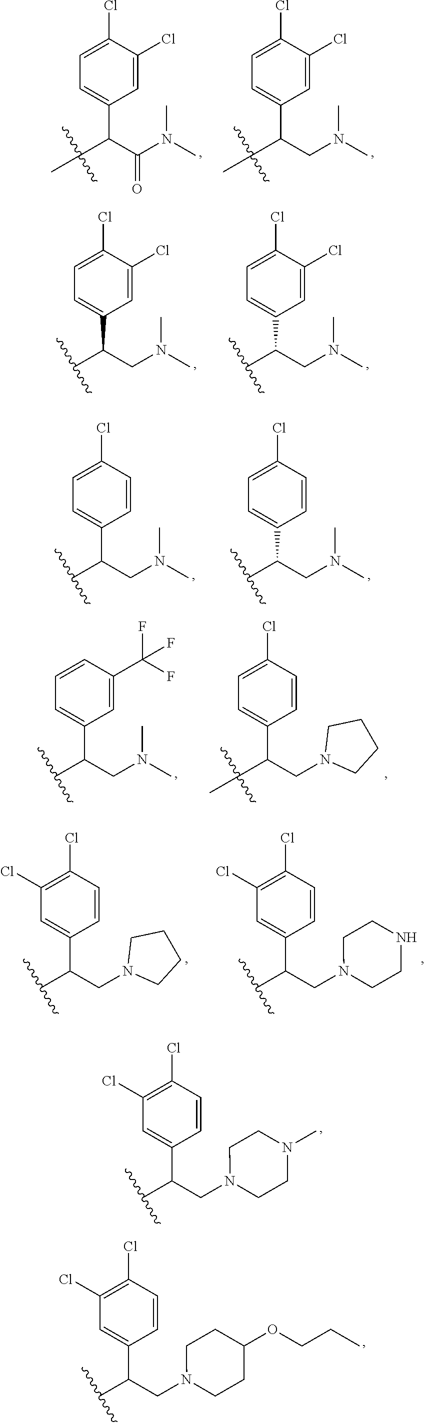

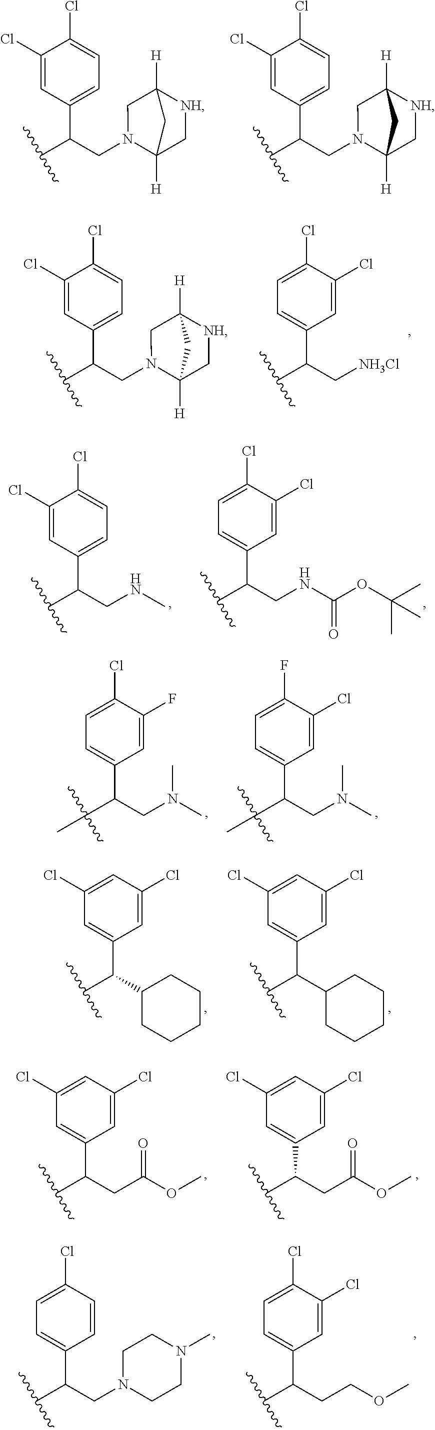

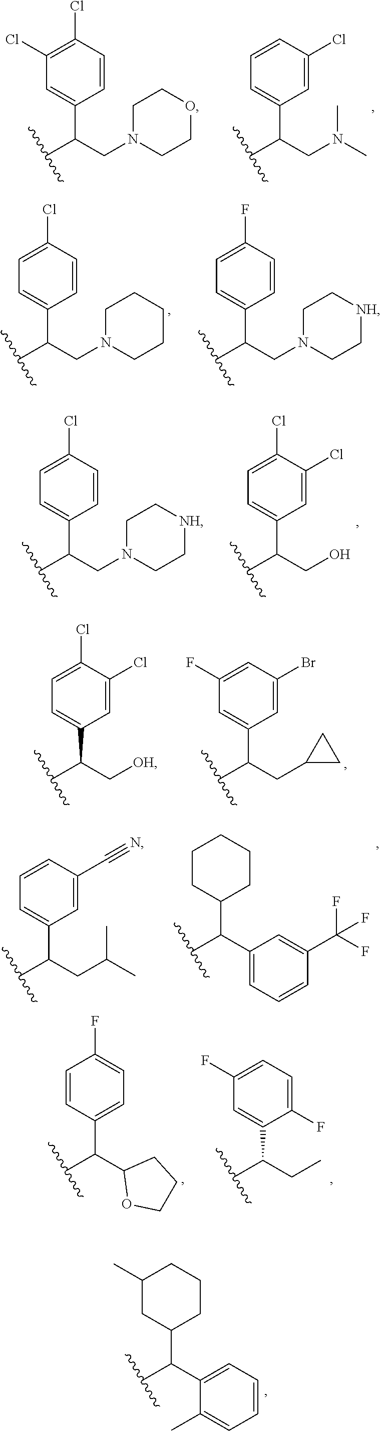

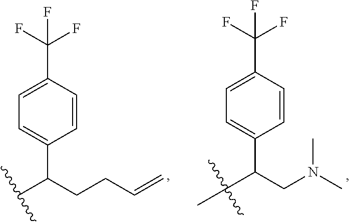 [SMILES: *C(CCC=C)c1ccc(C(F)(F)F)cc1 ; *C(CN(C)C)c1ccc(C(F)(F)F)cc1]

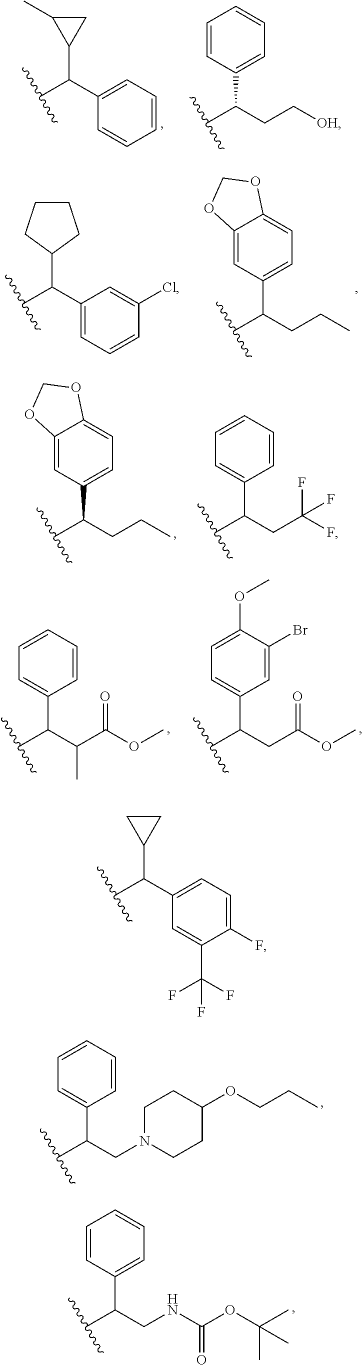

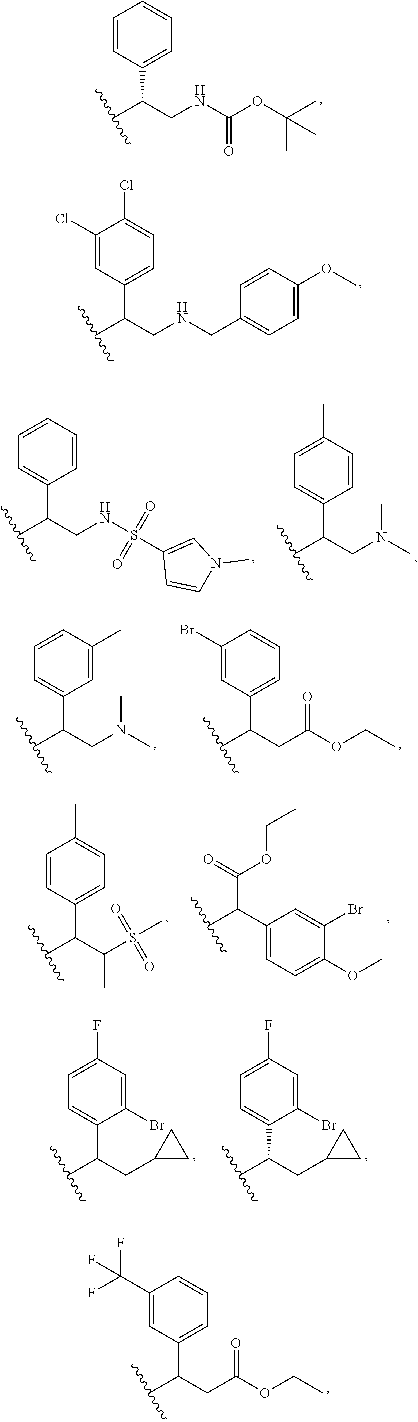

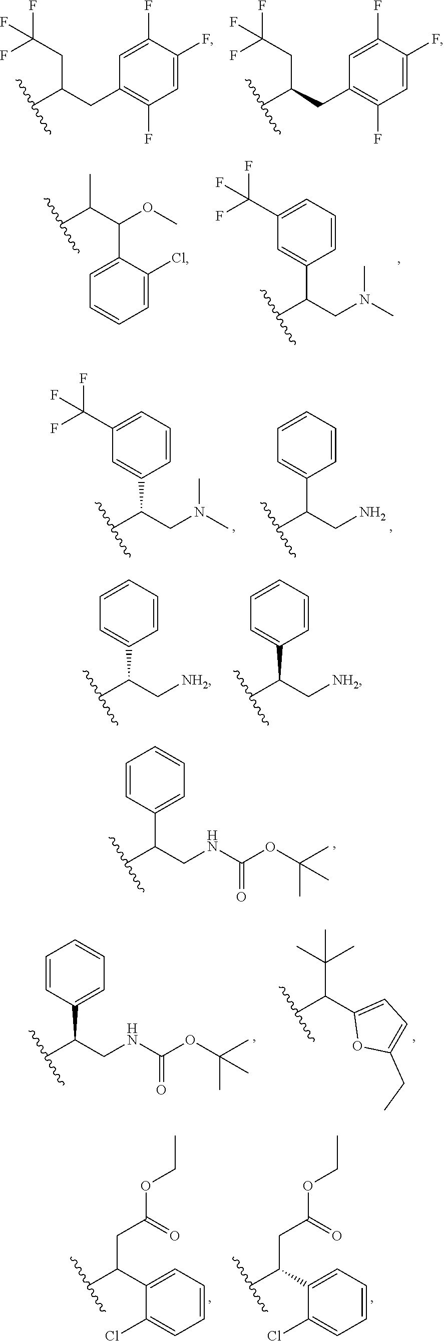

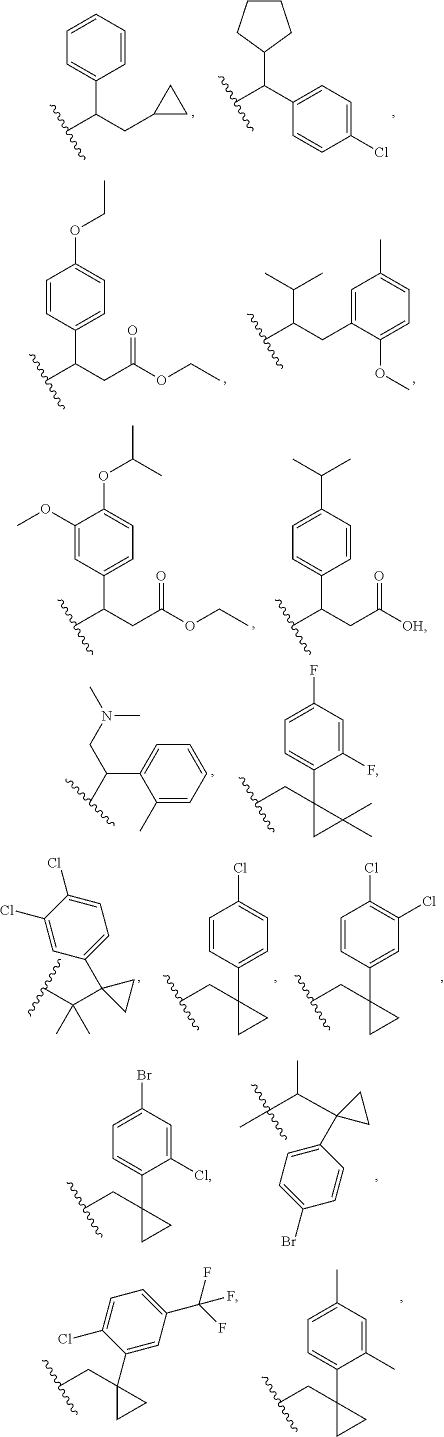

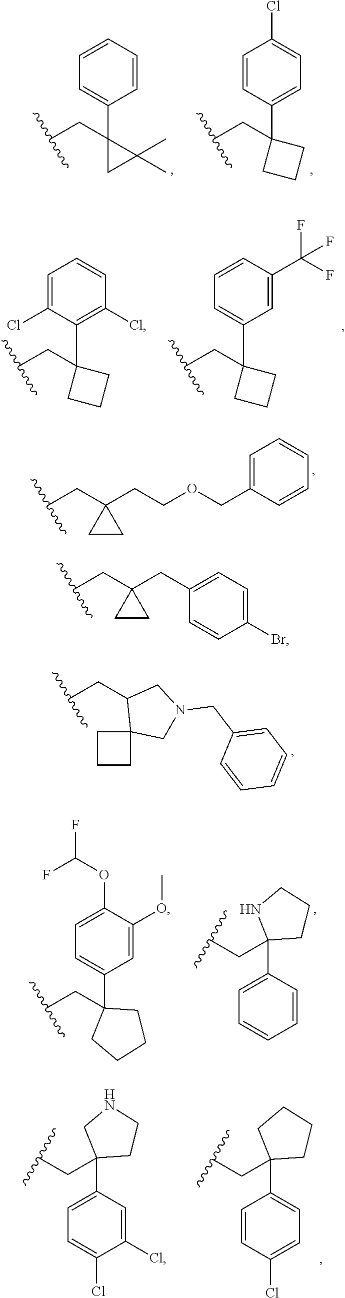

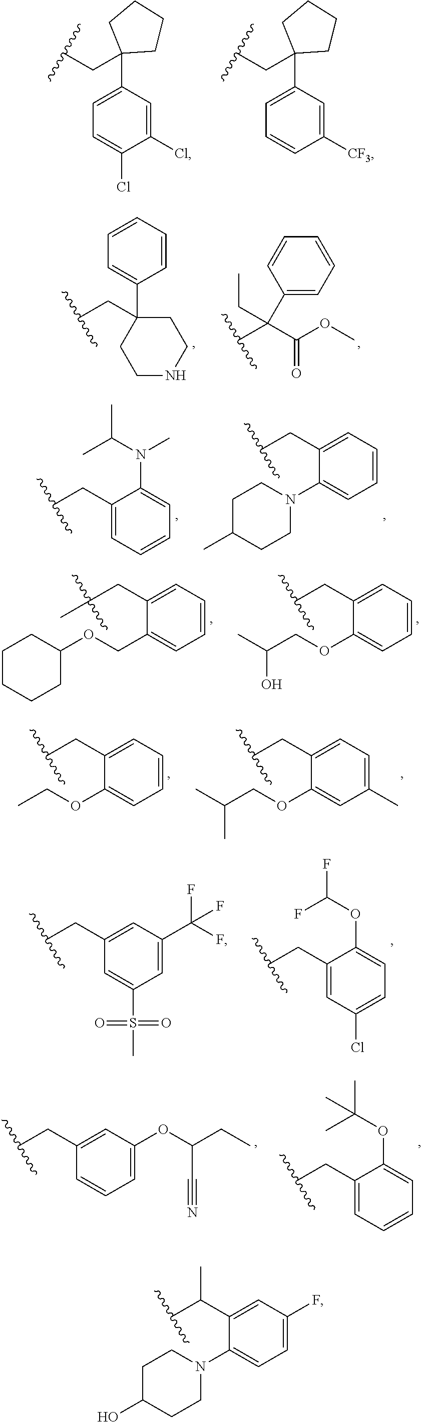

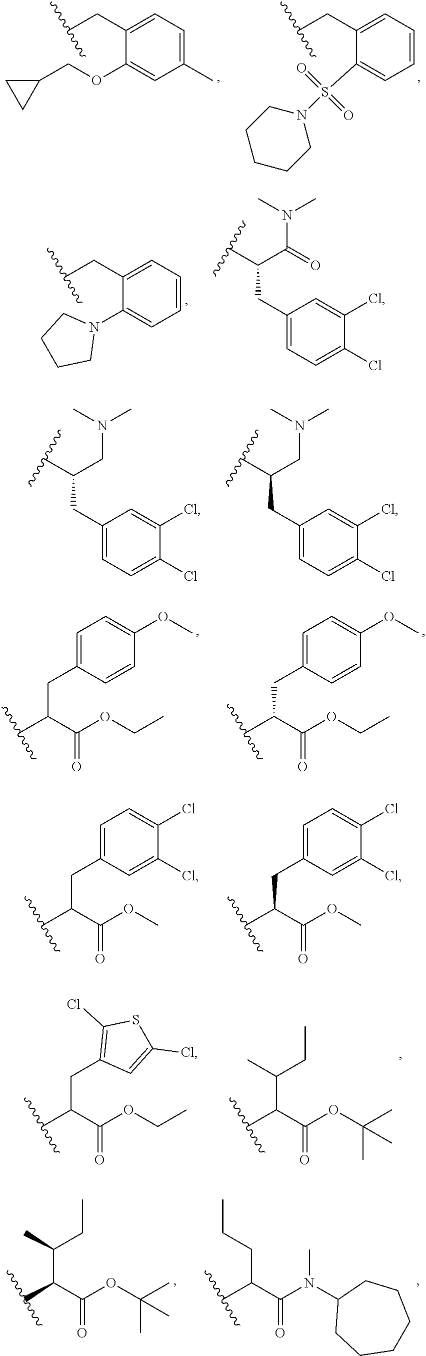

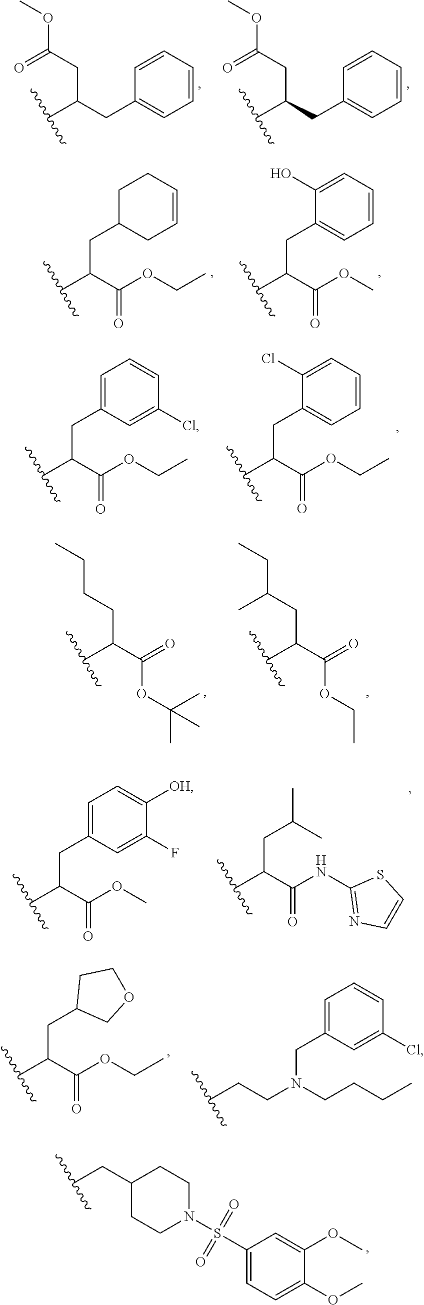

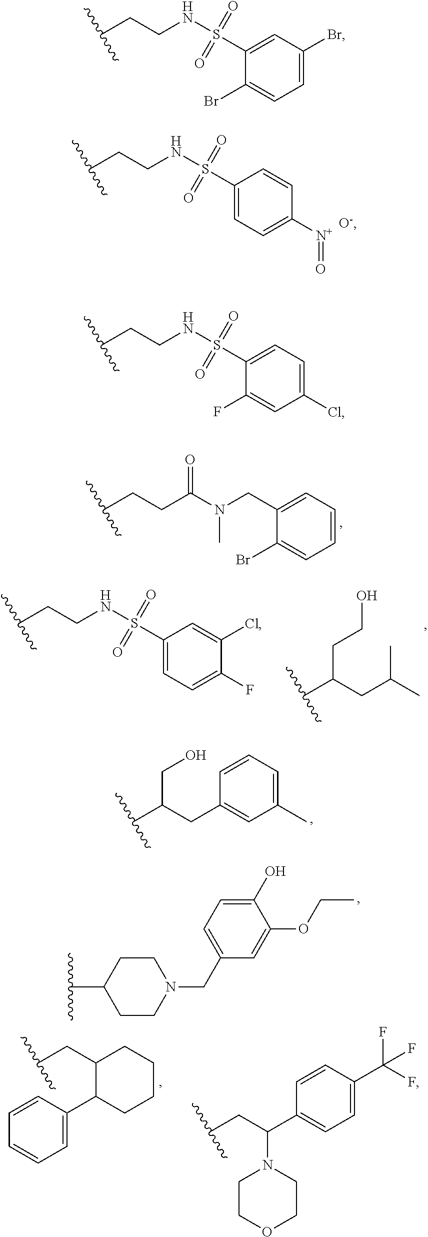

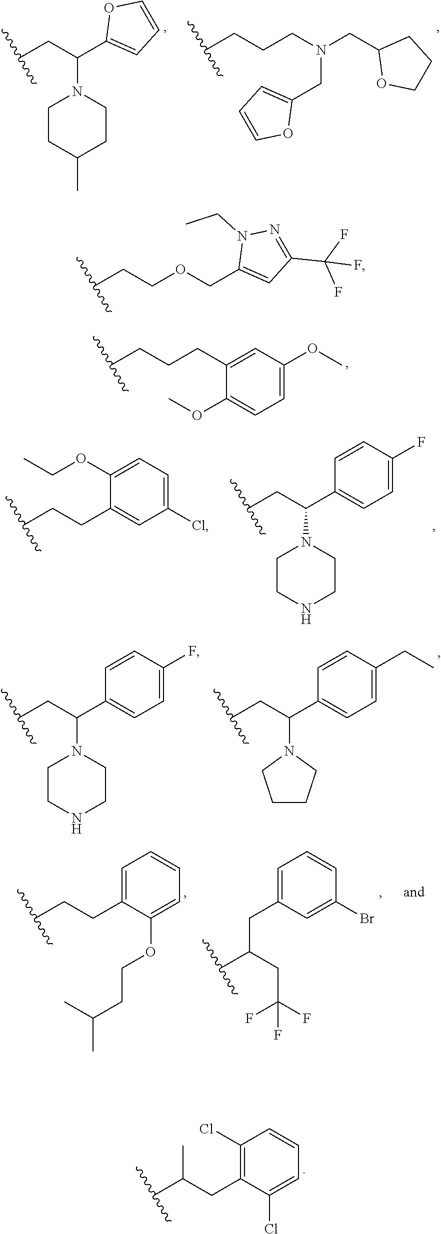

Embodiment 23 provides the compound of any one of Embodiments 15-17, wherein the compound of formula (II) is the compound of formula (IIb):

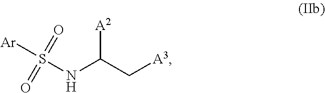

wherein:
A² is selected from the group consisting of

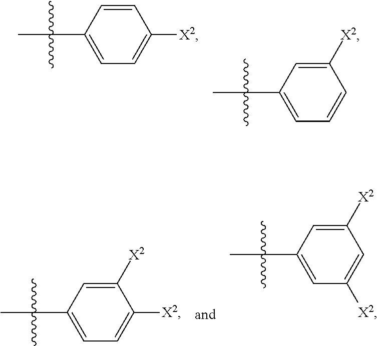

wherein each occurrence of X² is independently selected from the group consisting of H, F, Cl, Br, Me, and CF₃; and
A³ is selected from the group consisting of NMe₂, NH₃Cl, NHMe, NHC(═O)Ot-Bu,

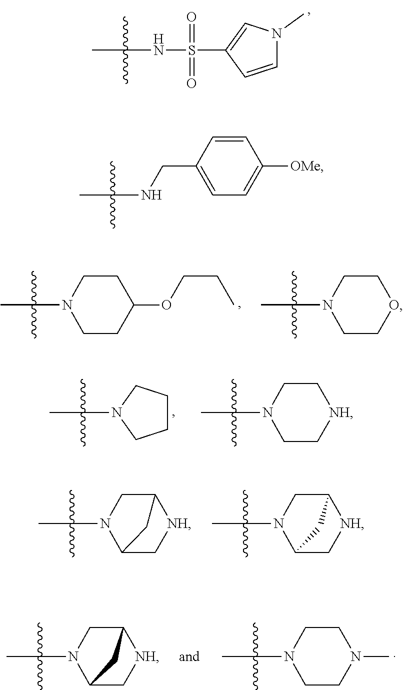

Embodiment 24 provides the compound of Embodiment 23 , wherein A³ is

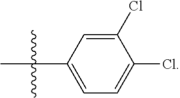 [SMILES: *c1ccc(Cl)c(Cl)c1]

Embodiment 25 provides the compound of Embodiment 23 or 24, wherein A³ is NMe₂.

Embodiment 26 provides the compound of any one of Embodiments 23-25, wherein Ar is selected from the group consisting of:

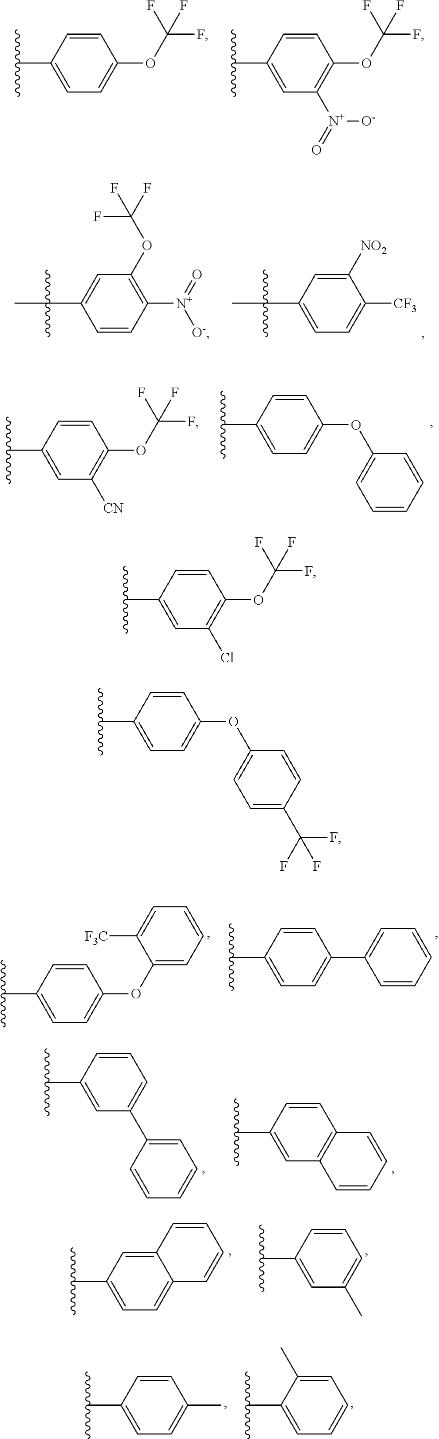

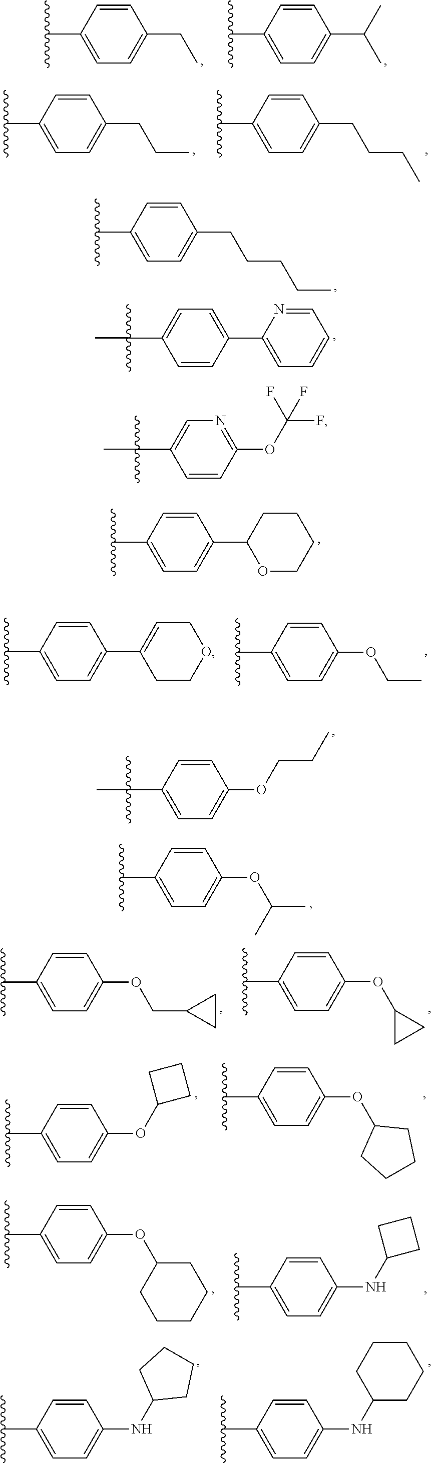

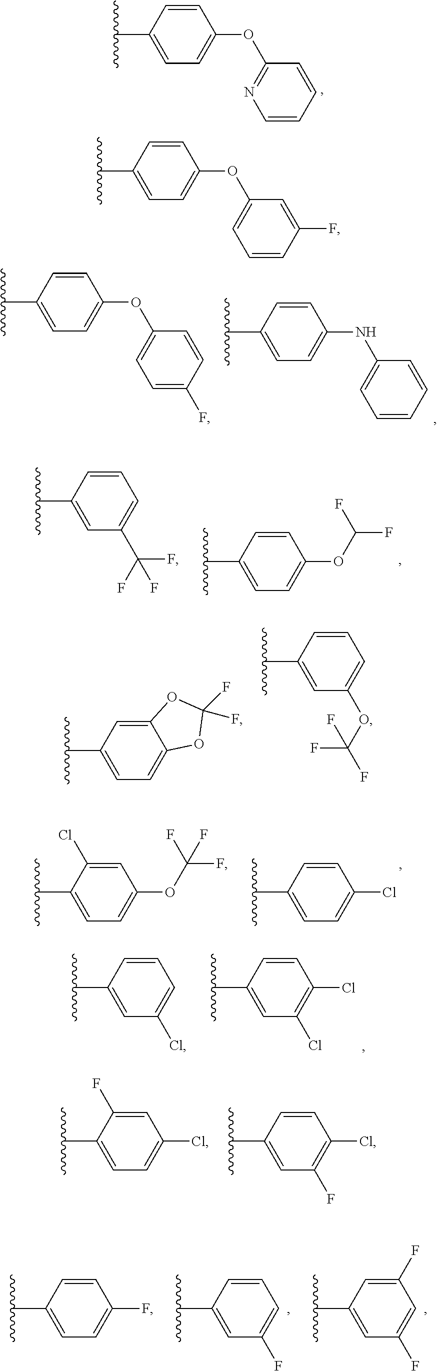

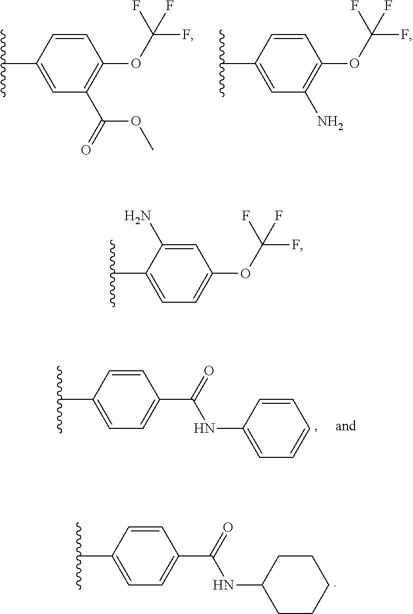

Embodiment 27 provides the compound of Embodiment 1, which is selected from the group consisting of:
N-(1-(3,4-dichlorophenyl)-2-(dimethylamino)ethyl)-3-nitro-4-(trifluoromethoxy)benzenesulfonamide;
N-(1-hydroxy-5-methylhexan-3-yl)-4-(trifluoromethoxy)benzenesulfonamide;
N-(1-(3,4-dichlorophenyl)-2-(dimethylamino)ethyl)-4-pentylbenzenesulfonamide;
N-(cyclohexyl(3,5-dichlorophenyl)methyl)-4-(trifluoromethoxy)benzenesulfonamide;
N-(1-(3,4-dichlorophenyl)-2-(dimethylamino)ethyl)-4-(4-(trifluoromethyl)phenoxy)benzenesulfonamide;
N-(1-(3,4-dichlorophenyl)-2-(dimethylamino)ethyl)-4-phenoxybenzenesulfonamide;
N-(1-(3,4-dichlorophenyl)-2-(dimethylamino)ethyl)-[1,1′-biphenyl]-4-sulfonamide;
N-(1-(4-chlorophenyl)-2-(dimethylamino)ethyl)-4-phenoxybenzenesulfonamide;
N-(2-(dimethylamino)-1-(3-(trifluoromethyl)phenyl)ethyl)-4-phenoxybenzenesulfonamide;
3-chloro-N-(1-(3,4-dichlorophenyl)-2-(dimethylamino)ethyl)-4-(trifluoromethoxy)benzenesulfonamide;
4-(cyclopentyloxy)-N-(1-(3,4-dichlorophenyl)-2-(dimethylamino)ethyl)benzenesulfonamide;
N-(1-(3,4-dichlorophenyl)-2-(dimethylamino)ethyl)-4-propylbenzenesulfonamide;
N-(1-(3,4-dichlorophenyl)-2-(4-methylpiperazin-1-yl)ethyl)-4-(trifluoromethoxy)benzenesulfonamide;
N-(1-(3,4-dichlorophenyl)-2-(dimethylamino)ethyl)-[1,1′-biphenyl]-3-sulfonamide;
3-chloro-N-(1-(4-chlorophenyl)-2-(dimethylamino)ethyl)-4-(trifluoromethoxy)benzenesulfonamide;
3-chloro-N-(2-(dimethylamino)-1-(3-(trifluoromethyl)phenyl)ethyl)-4-(trifluoromethoxy)benzenesulfonamide;
N-(1-(3-bromo-5-fluorophenyl)-2-cyclopropylethyl)-4-(trifluoromethoxy)benzenesulfonamide;
N-((1-(2,4-difluorophenyl)-2,2-dimethylcyclopropyl)methyl)-4-(trifluoromethoxy)benzenesulfonamide;
N-(1-(3,4-dichlorophenyl)-2-(dimethylamino)ethyl)-4-(trifluoromethoxy)benzenesulfonamide;
N-(2-(dimethylamino)-1-(3-(trifluoromethyl)phenyl)ethyl)-4-(4-(trifluoromethyl)phenoxy)benzenesulfonamide;
N-(2-(isopropyl(methyl)amino)benzyl)-4-(trifluoromethoxy)benzenesulfonamide;
4-(trifluoromethoxy)-N-(1-(4-(trifluoromethyl)phenyl)pent-4-en-1-yl)benzenesulfonamide;
N-(1-(3,4-dichlorophenyl)-2-(dimethylamino)ethyl)-3-(trifluoromethoxy)benzenesulfonamide;
2-amino-N-(1-(3,4-dichlorophenyl)-2-(dimethylamino)ethyl)-4-(trifluoromethoxy)benzenesulfonamide;
N-(1-(3,4-dichlorophenyl)-2-(dimethylamino)ethyl)naphthalene-1-sulfonamide;
N-(2-((cyclohexyloxy)methyl)benzyl)-4-(trifluoromethoxy)benzenesulfonamide;
N-((1-(4-bromo-2-chlorophenyl)cyclopropyl)methyl)-4-(trifluoromethoxy)benzenesulfonamide;
N-(2-(4-methylpiperidin-1-yl)benzyl)-4-(trifluoromethoxy)benzenesulfonamide;
N-(1-(4-chlorophenyl)-2-(4-methylpiperazin-1-yl)ethyl)-4-(trifluoromethoxy)benzenesulfonamide;
2-chloro-N-(1-(3,4-dichlorophenyl)-2-(dimethylamino)ethyl)-4-(trifluoromethoxy)benzenesulfonamide;
N-(1-(3,4-dichlorophenyl)-2-(dimethylamino)ethyl)naphthalene-2-sulfonamide;
3,4-dichloro-N-(1-(3,4-dichlorophenyl)-2-(dimethylamino)ethyl)benzenesulfonamide;
N-((2-(4-methoxyphenyl)-1-methylpyrrolidin-3-yl)methyl)-4-(trifluoromethoxy)benzenesulfonamide;
N-(1-hydroxy-3-(m-tolyl)propan-2-yl)-4-(trifluoromethoxy)benzenesulfonamide;
N-(1-(3,4-dichlorophenyl)-2-(dimethylamino)ethyl)-2-phenylethane-1-sulfonamide;
N-(1-(3,4-dichlorophenyl)-2-(pyrrolidin-1-yl)ethyl)-4-(trifluoromethoxy)benzenesulfonamide;
2,5-dibromo-N-(2-((4-(trifluoromethoxy)phenyl)sulfonamido)ethyl)benzenesulfonamide;
N-(1-(3,4-dichlorophenyl)-2-(dimethylamino)ethyl)-N′-methyl-4-(trifluoromethoxy)benzenesulfonimidamide;
N-(1-(3,4-dichlorophenyl)-2-(dimethylamino)ethyl)-2,2-difluorobenzo[d][1,3]dioxole-5-sulfonamide;
ethyl 3-((4-(trifluoromethoxy)phenyl)sulfonamido)-3-(3-(trifluoromethyl)phenyl)propanoate;
N-(1-(1-(4-bromophenyl)cyclopropyl)ethyl)-4-(trifluoromethoxy)benzenesulfonamide;
N-((1-(2-chloro-5-(trifluoromethyl)phenyl)cyclopropyl)methyl)-4-(trifluoromethoxy)benzenesulfonamide;
4-methyl-N-(thiazol-2-yl)-2-((4-(trifluoromethoxy)phenyl)sulfonamido)pentanamide;
N-(1-(3,4-dichlorophenyl)-2-(dimethylamino)ethyl)-4-isopropylbenzenesulfonamide;
N-(1-(3,4-dichlorophenyl)-2-(dimethylamino)ethyl)-4-(trifluoromethyl)benzenesulfonamide;
N-(4,4,4-trifluoro-1-(2,4,5-trifluorophenyl)butan-2-yl)-4-(trifluoromethoxy)benzenesulfonamide;
N-((6-benzyl-6-azaspiro[3.4]octan-8-yl)methyl)-4-(trifluoromethoxy)benzenesulfonamide;
N-(2-((1-ethyl-3-(trifluoromethyl)-1H-pyrazol-5-yl)methoxy)ethyl)-4-(trifluoromethoxy)benzenesulfonamide;
4-chloro-N-(1-(3,4-dichlorophenyl)-2-(dimethylamino)ethyl)benzenesulfonamide;
N-(1-(3-cyanophenyl)-3-methylbutyl)-4-(trifluoromethoxy)benzenesulfonamide;
N-(cyclohexyl(3-(trifluoromethyl)phenyl)methyl)-4-(trifluoromethoxy)benzenesulfonamide;
N-((1-(2,6-dichlorophenyl)cyclobutyl)methyl)-4-(trifluoromethoxy)benzenesulfonamide;
N-(2-(cyclopropylmethoxy)-4-methylbenzyl)-4-(trifluoromethoxy)benzenesulfonamide;
(E)-N-(1-(3,4-dichlorophenyl)-2-(dimethylamino)ethyl)-2-phenylethene-1-sulfonamide;
N-(2-(2-hydroxypropoxy)benzyl)-4-(trifluoromethoxy)benzenesulfonamide;
3-chloro-4-fluoro-N-(2-((4-(trifluoromethoxy)phenyl)sulfonamido)ethyl)benzenesulfonamide;
3-amino-N-(1-(3,4-dichlorophenyl)-2-(dimethylamino)ethyl)-4-(trifluoromethoxy)benzenesulfonamide;
N-(2-(butyl(3-chlorobenzyl)amino)ethyl)-4-(trifluoromethoxy)benzenesulfonamide;
N-((4-fluorophenyl)(tetrahydrofuran-2-yl)methyl)-4-(trifluoromethoxy)benzenesulfonamide;
N-(1-(4-chlorophenyl)-2-(piperidin-1-yl)ethyl)-4-(trifluoromethoxy)benzenesulfonamide;
N-(1-(3,4-dichlorophenyl)-2-(dimethylamino)ethyl)-4-ethylbenzenesulfonamide;
N-(1-(2,5-difluorophenyl)propyl)-4-(trifluoromethoxy)benzenesulfonamide;
methyl 3-(3,5-dichlorophenyl)-3-((4-(trifluoromethoxy)phenyl)sulfonamido)propanoate;
N-((3-chlorophenyl)(cyclopentyl)methyl)-4-(trifluoromethoxy)benzenesulfonamide;
4-(trifluoromethoxy)-N-((1-(3-(trifluoromethyl)phenyl)cyclobutyl)methyl)benzenesulfonamide;
N-(1-(benzo[d][1,3]dioxol-5-yl)butyl)-4-(trifluoromethoxy)benzenesulfonamide;
methyl 3-(3-fluoro-4-hydroxyphenyl)-2-((4-(trifluoromethoxy)phenyl)sulfonamido)propanoate;
N-(5-chloro-2-ethoxyphenethyl)-4-(trifluoromethoxy)benzenesulfonamide;
N-((2-phenylcyclohexyl)methyl)-4-(trifluoromethoxy)benzenesulfonamide;
methyl 3-(3-bromo-4-methoxyphenyl)-3-((4-(trifluoromethoxy)phenyl)sulfonamido)propanoate;
tert-butyl 2-((4-(trifluoromethoxy)phenyl)sulfonamido)hexanoate;
N-((1-(4-bromobenzyl)cyclopropyl)methyl)-4-(trifluoromethoxy)benzenesulfonamide;
N-(cyclopropyl(4-fluoro-3-(trifluoromethyl)phenyl)methyl)-4-(trifluoromethoxy)benzenesulfonamide;
N-(1-(3,4-dichlorophenyl)-2-hydroxyethyl)-4-(trifluoromethoxy)benzenesulfonamide;
N-(3-(2,5-dimethoxyphenyl)propyl)-4-(trifluoromethoxy)benzenesulfonamide;
N-(1-(3,4-dichlorophenyl)-2-(dimethylamino)ethyl)-3-methylbenzenesulfonamide;
4-chloro-2-fluoro-N-(2-((4-(trifluoromethoxy)phenyl)sulfonamido)ethyl)benzenesulfonamide;
methyl 2-(2-bromophenyl)-2-((4-(trifluoromethoxy)phenyl)sulfonamido)acetate;
tert-butyl 4,4-difluoro-2-(((4-(trifluoromethoxy)phenyl)sulfonamido)methyl)pyrrolidine-1-carboxylate;
N-(2-bromobenzyl)-N-methyl-3-((4-(trifluoromethoxy)phenyl)sulfonamido)propanamide;
N-((1-(2,4-dimethylphenyl)cyclopropyl)methyl)-4-(trifluoromethoxy)benzenesulfonamide;
N-(1-(3-chlorophenyl)-2-(dimethylamino)ethyl)-4-(trifluoromethoxy)benzenesulfonamide;
N-(2-morpholino-2-(4-(trifluoromethyl)phenyl)ethyl)-4-(trifluoromethoxy)benzenesulfonamide;
N-(2-(dimethylamino)-1-(p-tolyl)ethyl)-4-(trifluoromethoxy)benzenesulfonamide;
N-(1-(3,4-dichlorophenyl)-2-(dimethylamino)ethyl)-4-(difluoromethoxy)benzenesulfonamide;
N-(2-(furan-2-yl)-2-(4-methylpiperidin-1-yl)ethyl)-4-(trifluoromethoxy)benzenesulfonamide;
ethyl 3-(2-chlorophenyl)-2-((4-(trifluoromethoxy)phenyl)sulfonamido)propanoate;
N-(2-(tert-butoxy)benzyl)-4-(trifluoromethoxy)benzenesulfonamide;
3-chloro-N-(1-(3,4-dichlorophenyl)-2-(dimethylamino)ethyl)benzenesulfonamide;
N-(2-(dimethylamino)-1-(3-(trifluoromethyl)phenyl)ethyl)-4-(trifluoromethoxy)benzenesulfonamide;
N-(1-(3,4-dichlorophenyl)-2-(dimethylamino)ethyl)-4-(trifluoromethoxy)benzenesulfonimidamide;
N-(1-(5-fluoro-2-(4-hydroxypiperidin-1-yl)phenyl)ethyl)-4-(trifluoromethoxy)benzenesulfonamide;
N-(2-(dimethylamino)-1-(m-tolyl)ethyl)-4-(trifluoromethoxy)benzenesulfonamide;
ethyl 3-(3-bromophenyl)-3-((4-(trifluoromethoxy)phenyl)sulfonamido)propanoate;
N-(1-(3,4-dichlorophenyl)-2-(dimethylamino)ethyl)-2-methylbenzenesulfonamide;
4-chloro-N-(1-(3,4-dichlorophenyl)-2-(dimethylamino)ethyl)-3-fluorobenzenesulfonamide;
ethyl 3-(3-chlorophenyl)-2-((4-(trifluoromethoxy)phenyl)sulfonamido)propanoate;
N-(1-(2-bromo-4-fluorophenyl)-2-cyclopropylethyl)-4-(trifluoromethoxy)benzenesulfonamide;
N-(2-(pyrrolidin-1-yl)benzyl)-4-(trifluoromethoxy)benzenesulfonamide;
ethyl 2-(3-bromo-4-methoxyphenyl)-2-((4-(trifluoromethoxy)phenyl)sulfonamido)acetate;
methyl 3-(2-hydroxyphenyl)-2-((4-(trifluoromethoxy)phenyl)sulfonamido)propanoate;
N-(1-(3,4-dichlorophenyl)-3-methoxypropyl)-4-(trifluoromethoxy)benzenesulfonamide;
N-(1-(2-chlorophenyl)-1-methoxypropan-2-yl)-4-(trifluoromethoxy)benzenesulfonamide;
N-(1-(3,4-dichlorophenyl)-2-(dimethylamino)ethyl)-4-ethoxybenzenesulfonamide;
N-(1-(4-chlorophenyl)-2-(pyrrolidin-1-yl)ethyl)-4-(trifluoromethoxy)benzenesulfonamide;
N-(1-(3,4-dichlorophenyl)-2-(dimethylamino)ethyl)-4-methylbenzenesulfonamide;
N′-(1-(3,4-dichlorophenyl)-2-(dimethylamino)ethyl)-N-methyl-4-(trifluoromethoxy)benzenesulfonimidamide;
N-(1-(3,4-dichlorophenyl)-2-(dimethylamino)ethyl)-3-(trifluoromethyl)benzenesulfonamide;
ethyl 3-(cyclohex-3-en-1-yl)-2-((4-(trifluoromethoxy)phenyl)sulfonamido)propanoate;
N-cyclohexyl-4-(N-(1-(3,4-dichlorophenyl)-2-(dimethylamino)ethyl)sulfamoyl)benzamide;
N-(1-(3,4-dichlorophenyl)-2-morpholinoethyl)-4-(trifluoromethoxy)benzenesulfonamide;
N-cycloheptyl-N-methyl-2-((4-(trifluoromethoxy)phenyl)sulfonamido)pentanamide;
N-(4,4,4-trifluoro-3-phenylbutyl)-4-(trifluoromethoxy)benzenesulfonamide;
N-(1-(5-ethylfuran-2-yl)-2,2-dimethylpropyl)-4-(trifluoromethoxy)benzenesulfonamide;
N-(2-(4-ethylphenyl)-2-(pyrrolidin-1-yl)ethyl)-4-(trifluoromethoxy)benzenesulfonamide;
N-(2-(isopentyloxy)phenethyl)-4-(trifluoromethoxy)benzenesulfonamide;
N-(1-(4-chlorophenyl)-2-(dimethylamino)ethyl)-4-(trifluoromethoxy)benzenesulfonamide;
methyl 3-(3,4-dichlorophenyl)-2-((4-(trifluoromethoxy)phenyl)sulfonamido)propanoate;
N-(1-(3,4-dichlorophenyl)-2-(dimethylamino)ethyl)cyclohexanesulfonamide;
ethyl 3-(2,5-dichlorothiophen-3-yl)-2-((4-(trifluoromethoxy)phenyl)sulfonamido)propanoate;
tert-butyl 3-methyl-2-((4-(trifluoromethoxy)phenyl)sulfonamido)pentanoate;
N-(2-(piperidin-1-ylsulfonyl)benzyl)-4-(trifluoromethoxy)benzenesulfonamide;
4-chloro-N-(1-(3,4-dichlorophenyl)-2-(dimethylamino)ethyl)-2-fluorobenzenesulfonamide;
N-(1-(3-bromophenyl)-4,4,4-trifluorobutan-2-yl)-4-(trifluoromethoxy)benzenesulfonamide;
N-(2-(dimethylamino)-1-(o-tolyl)ethyl)-4-(trifluoromethoxy)benzenesulfonamide;
N-(2-ethoxybenzyl)-4-(trifluoromethoxy)benzenesulfonamide;
N-(2-isobutoxy-4-methylbenzyl)-4-(trifluoromethoxy)benzenesulfonamide;
ethyl 3-(tetrahydrofuran-3-yl)-2-((4-(trifluoromethoxy)phenyl)sulfonamido)propanoate;
methyl 4-phenyl-3-((4-(trifluoromethoxy)phenyl)sulfonamido)butanoate;
N-(1-(3,4-dichlorophenyl)-2-(dimethylamino)ethyl)-4-fluorobenzenesulfonamide;
N-((1-((3,4-dimethoxyphenyl)sulfonyl)piperidin-4-yl)methyl)-4-(trifluoromethoxy)benzenesulfonamide;
N-(3-(methylsulfonyl)-5-(trifluoromethyl)benzyl)-4-(trifluoromethoxy)benzenesulfonamide;
4-nitro-N-(2-((4-(trifluoromethoxy)phenyl)sulfonamido)ethyl)benzenesulfonamide;
3-(4-isopropylphenyl)-3-((4-(trifluoromethoxy)phenyl)sulfonamido)propanoic acid;
methyl 2-phenyl-2-((4-(trifluoromethoxy)phenyl)sulfonamido)butanoate;
N-(1-(3,4-dichlorophenyl)-2-(dimethylamino)ethyl)-3-fluorobenzenesulfonamide;
N-(5-chloro-2-(difluoromethoxy)benzyl)-4-(trifluoromethoxy)benzenesulfonamide;
ethyl 4-methyl-2-((4-(trifluoromethoxy)phenyl)sulfonamido)hexanoate;
N-(3-(1-cyanopropoxy)benzyl)-4-(trifluoromethoxy)benzenesulfonamide;
ethyl 3-(4-methoxyphenyl)-2-((4-(trifluoromethoxy)phenyl)sulfonamido)propanoate;
ethyl 3-(4-isopropoxy-3-methoxyphenyl)-3-((4-(trifluoromethoxy)phenyl)sulfonamido)propanoate;
N-((1-(4-(difluoromethoxy)-3-methoxyphenyl)cyclopentyl)methyl)-4-(trifluoromethoxy)benzenesulfonamide;
N-((4-chlorophenyl)(cyclopentyl)methyl)-4-(trifluoromethoxy)benzenesulfonamide;
ethyl 3-(4-ethoxyphenyl)-3-((4-(trifluoromethoxy)phenyl)sulfonamido)propanoate;
4-cyclopropoxy-N-(1-(3,4-dichlorophenyl)-2-(dimethylamino)ethyl)benzenesulfonamide;
N-(1-(3-chloro-4-fluorophenyl)-2-(dimethylamino)ethyl)-4-(trifluoromethoxy)benzenesulfonamide;
N-(1-(4-chloro-3-fluorophenyl)-2-(dimethylamino)ethyl)-4-(trifluoromethoxy)benzenesulfonamide;
4-(N-(1-(3,4-dichlorophenyl)-2-(dimethylamino)ethyl)sulfamoyl)-N-phenylbenzamide;
N-(1-(3,4-dichlorophenyl)-2-(dimethylamino)ethyl)-4-(phenylamino)benzenesulfonamide;
N-(1-(4-chlorophenyl)-2-(dimethylamino)ethyl)-4-(4-(trifluoromethyl)phenoxy)benzenesulfonamide;
N-(2-(dimethylamino)-1-(3-(trifluoromethyl)phenyl)ethyl)-3-nitro-4-(trifluoromethoxy)benzenesulfonamide;
N-(1-(4-chlorophenyl)-2-(dimethylamino)ethyl)-3-nitro-4-(trifluoromethoxy)benzenesulfonamide;
4-(cyclopropylmethoxy)-N-(1-(3,4-dichlorophenyl)-2-(dimethylamino)ethyl)benzenesulfonamide;
N-(1-(3,4-dichlorophenyl)-2-(dimethylamino)ethyl)-4-isopropoxybenzenesulfonamide;
4-(cyclohexyloxy)-N-(1-(3,4-dichlorophenyl)-2-(dimethylamino)ethyl)benzenesulfonamide;
N-(1-(3,4-dichlorophenyl)-2-(dimethylamino)ethyl)-4-(pyridin-2-yloxy)benzenesulfonamide;
4-butyl-N-(1-(3,4-dichlorophenyl)-2-(dimethylamino)ethyl)benzenesulfonamide;
N-(1-(3,4-dichlorophenyl)-2-(dimethylamino)ethyl)-4-(3,6-dihydro-2H-pyran-4-yl)benzenesulfonamide;
N-(2-(dimethylamino)-1-(4-(trifluoromethyl)phenyl)ethyl)-4-(trifluoromethoxy)benzenesulfonamide;
N-(1-(3,4-dichlorophenyl)-2-(dimethylamino)ethyl)-4-(3-fluorophenoxy)benzenesulfonamide;
N-(1-(3,4-dichlorophenyl)-2-(dimethylamino)ethyl)-4-(4-fluorophenoxy)benzenesulfonamide;
methyl 5-(N-(1-(3,4-dichlorophenyl)-2-(dimethylamino)ethyl)sulfamoyl)-2-(trifluoromethoxy)benzoate;
N-(1-(3,4-dichlorophenyl)-2-(4-methylpiperazin-1-yl)ethyl)-4-(4-(trifluoromethyl)phenoxy)benzenesulfonamide;
N-(1-(4-chlorophenyl)-2-(4-methylpiperazin-1-yl)ethyl)-4-phenoxybenzenesulfonamide;
N-(1-(4-chlorophenyl)-2-(4-methylpiperazin-1-yl)ethyl)-4-(4-(trifluoromethyl)phenoxy)benzenesulfonamide;
N-(1-(3,4-dichlorophenyl)-2-(4-methylpiperazin-1-yl)ethyl)-4-phenoxybenzenesulfonamide;
N-(2-(dimethylamino)-1-(3-(trifluoromethyl)phenyl)ethyl)-4-(4-(trifluoromethyl)phenoxy)benzenesulfonamide;
N-(1-(3,4-dichlorophenyl)-2-(dimethylamino)ethyl)-6-(trifluoromethoxy)pyridine-3-sulfonamide;
N-(1-(3,4-dichlorophenyl)-2-(dimethylamino)ethyl)-3-nitro-4-(trifluoromethoxy)benzenesulfonamide;
N-(1-(3,4-dichlorophenyl)-2-(dimethylamino)ethyl)-4-(pyridin-2-yl)benzenesulfonamide;
N-(1-(3,4-dichlorophenyl)-2-(dimethylamino)ethyl)-4-nitro-3-(trifluoromethoxy)benzenesulfonamide;
N-(2-amino-1-(3,4-dichlorophenyl)ethyl)-4-(trifluoromethoxy)benzenesulfonamide hydrochloride;
tert-butyl (2-(3,4-dichlorophenyl)-2-((4-(trifluoromethoxy)phenyl)sulfonamido)ethyl)carbamate;
N-((1-(4-chlorophenyl)cyclopropyl)methyl)-4-(trifluoromethoxy)benzenesulfonamide;
N-((1-(4-chlorophenyl)cyclobutyl)methyl)-4-(trifluoromethoxy)benzenesulfonamide;
N-((2,2-dimethyl-1-phenylcyclopropyl)methyl)-4-(trifluoromethoxy)benzenesulfonamide;
N-(1-(3,4-dichlorophenyl)-2-(dimethylamino)ethyl)-2-nitro-3-(trifluoromethoxy)benzenesulfonamide;
N-((1-(3,4-dichlorophenyl)cyclopropyl)methyl)-4-(trifluoromethoxy)benzenesulfonamide;
N-((1-(3,4-dichlorophenyl)cyclopropyl)methyl)-4-(4-(trifluoromethyl)phenoxy)benzenesulfonamide;
N-((1-(3,4-dichlorophenyl)cyclopropyl)methyl)-3-nitro-4-(trifluoromethoxy)benzenesulfonamide;
N-(1-(3,4-dichlorophenyl)-2-(piperazin-1-yl)ethyl)-4-(trifluoromethoxy)benzenesulfonamide;
N-(1-(3,4-dichlorophenyl)-2-(dimethylamino)ethyl)-3,5-difluorobenzenesulfonamide;
N-(1-(2-methoxy-5-methylphenyl)-3-methylbutan-2-yl)-4-(trifluoromethoxy)benzenesulfonamide;
N-(1-(3,4-dichlorophenyl)-2-(dimethylamino)ethyl)-4-(2-(trifluoromethyl)phenoxy)benzenesulfonamide;
N-(1-(3,4-dichlorophenyl)-2-(dimethylamino)ethyl)-4-(tetrahydro-2H-pyran-2-yl)benzenesulfonamide;
3-cyano-N-(1-(3,4-dichlorophenyl)-2-(dimethylamino)ethyl)-4-(trifluoromethoxy)benzenesulfonamide;
N-(1-(3,4-dichlorophenyl)-2-(4-propoxypiperidin-1-yl)ethyl)-4-(trifluoromethoxy)benzenesulfonamide;
N-(1-(3,4-dichlorophenyl)-2-(methylamino)ethyl)-4-(trifluoromethoxy)benzenesulfonamide;
N-((1-(3,4-dichlorophenyl)cyclopentyl)methyl)-4-(trifluoromethoxy)benzenesulfonamide;
N-((1-(4-chlorophenyl)cyclopentyl)methyl)-4-(trifluoromethoxy)benzenesulfonamide;
N-(2-(1-(3,4-dichlorophenyl)cyclopropyl)propan-2-yl)-4-(trifluoromethoxy)benzenesulfonamide;
N-(1-(3,4-dichlorophenyl)-3-(dimethylamino)propan-2-yl)-4-(trifluoromethoxy)benzenesulfonamide;
4-cyclobutoxy-N-(1-(3,4-dichlorophenyl)-2-(dimethylamino)ethyl)benzenesulfonamide;
3-(3,4-dichlorophenyl)-N,N-dimethyl-2-((4-(trifluoromethoxy)phenyl)sulfonamido)propenamide;
N-(1-(3,4-dichlorophenyl)-2-(dimethylamino)ethyl)-N-methyl-4-(trifluoromethoxy)benzenesulfonamide;
4-(cyclopentylamino)-N-(1-(3,4-dichlorophenyl)-2-(dimethylamino)ethyl)benzenesulfonamide;
4-(cyclobutylamino)-N-(1-(3,4-dichlorophenyl)-2-(dimethylamino)ethyl)benzenesulfonamide;
N-((3-(3,4-dichlorophenyl)pyrrolidin-3-yl)methyl)-4-(trifluoromethoxy)benzenesulfonamide;
2-(3,4-dichlorophenyl)-N,N-dimethyl-2-((4-(trifluoromethoxy)phenyl)sulfonamido)acetamide;
N-(1-(3,4-dichlorophenyl)-2-((4-methoxybenzyl)amino)ethyl)-4-(trifluoromethoxy)benzenesulfonamide;
N-(1-(4-chlorophenyl)-2-(piperazin-1-yl)ethyl)-4-(trifluoromethoxy)benzenesulfonamide;
N-(1-(4-chloro-3-fluorophenyl)-2-(piperazin-1-yl)ethyl)-4-(trifluoromethoxy)benzenesulfonamide;
N-(1-(4-chlorophenyl)-2-(piperazin-1-yl)ethyl)-6-(trifluoromethoxy)pyridine-3-sulfonamide;
N-((4-phenylpiperidin-4-yl)methyl)-4-(trifluoromethoxy)benzenesulfonamide;
N-(2-(2,5-diazabicyclo[2.2.1]heptan-2-yl)-1-(3,4-dichlorophenyl)ethyl)-4-(trifluoromethoxy)benzenesulfonamide;
4-(trifluoromethoxy)-N-((1-(3-(trifluoromethyl)phenyl)cyclopentyl)methyl)benzenesulfonamide;
N-((2-phenylpyrrolidin-2-yl)methyl)-4-(trifluoromethoxy)benzenesulfonamide;
N-(1-(4-fluorophenyl)-2-(piperazin-1-yl)ethyl)-4-(trifluoromethoxy)benzenesulfonamide;
N-(2-(4-fluorophenyl)-2-(piperazin-1-yl)ethyl)-4-(trifluoromethoxy)benzenesulfonamide;
N-(1-(3,4-dichlorophenyl)-2-(dimethylamino)ethyl)-3-nitro-4-(trifluoromethyl)benzenesulfonamide;
N-(1-(3,4-dichlorophenyl)-2-(dimethylamino)ethyl)-4-propoxybenzenesulfonamide;
4-(cyclohexylamino)-N-(1-(3,4-dichlorophenyl)-2-(dimethylamino)ethyl)benzenesulfonamide; and
N-(1-(3,4-dichlorophenyl)-2-(4-methylpiperazin-1-yl)ethyl)-4-(4-fluorophenoxy)benzenesulfonamide;
or a salt, solvate, isotopologue, or tautomer thereof.

Embodiment 28 provides the compound of Embodiment 1, which is selected from the group consisting of
(R)—N-(1-(3,4-dichlorophenyl)-2-(dimethylamino)ethyl)-3-nitro-4-(trifluoromethoxy)benzenesulfonamide;
(R)—N-(1-hydroxy-5-methylhexan-3-yl)-4-(trifluoromethoxy)benzenesulfonamide;
(R)—N-(1-(3,4-dichlorophenyl)-2-(dimethylamino)ethyl)-4-pentylbenzenesulfonamide;
(R)—N-(cyclohexyl(3,5-dichlorophenyl)methyl)-4-(trifluoromethoxy)benzenesulfonamide;
(R)—N-(1-(3,4-dichlorophenyl)-2-(dimethylamino)ethyl)-4-(4-(trifluoromethyl)phenoxy)benzenesulfonamide;
(R)—N-(1-(3,4-dichlorophenyl)-2-(dimethylamino)ethyl)-4-phenoxybenzenesulfonamide;
(R)—N-(1-(3,4-dichlorophenyl)-2-(dimethylamino)ethyl)-[1,1′-biphenyl]-4-sulfonamide;
(R)—N-(1-(4-chlorophenyl)-2-(dimethylamino)ethyl)-4-phenoxybenzenesulfonamide;
(R)—N-(2-(dimethylamino)-1-(3-(trifluoromethyl)phenyl)ethyl)-4-phenoxybenzenesulfonamide;
(R)-3-chloro-N-(1-(3,4-dichlorophenyl)-2-(dimethylamino)ethyl)-4-(trifluoromethoxy)benzenesulfonamide;
(R)-4-(cyclopentyloxy)-N-(1-(3,4-dichlorophenyl)-2-(dimethylamino)ethyl)benzenesulfonamide;
(R)—N-(1-(3,4-dichlorophenyl)-2-(dimethylamino)ethyl)-4-propylbenzenesulfonamide;
(R)—N-(1-(3,4-dichlorophenyl)-2-(4-methylpiperazin-1-yl)ethyl)-4-(trifluoromethoxy)benzenesulfonamide;
(R)—N-(1-(3,4-dichlorophenyl)-2-(dimethylamino)ethyl)-[1,1′-biphenyl]-3-sulfonamide;
(R)-3-chloro-N-(1-(4-chlorophenyl)-2-(dimethylamino)ethyl)-4-(trifluoromethoxy)benzenesulfonamide;
(R)-3-chloro-N-(2-(dimethylamino)-1-(3-(trifluoromethyl)phenyl)ethyl)-4-(trifluoromethoxy)benzenesulfonamide;
(R)—N-(1-(3-bromo-5-fluorophenyl)-2-cyclopropylethyl)-4-(trifluoromethoxy)benzenesulfonamide;
(R)—N-((1-(2,4-difluorophenyl)-2,2-dimethylcyclopropyl)methyl)-4-(trifluoromethoxy)benzenesulfonamide;
(R)—N-(1-(3,4-dichlorophenyl)-2-(dimethylamino)ethyl)-4-(trifluoromethoxy)benzenesulfonamide;
(R)—N-(2-(dimethylamino)-1-(3-(trifluoromethyl)phenyl)ethyl)-4-(4-(trifluoromethyl)phenoxy)benzenesulfonamide;
(R)—N-(2-(isopropyl(methyl)amino)benzyl)-4-(trifluoromethoxy)benzenesulfonamide;
(R)-4-(trifluoromethoxy)-N-(1-(4-(trifluoromethyl)phenyl)pent-4-en-1-yl)benzenesulfonamide;
N—((R)-((1R,2R)-2-methylcyclopropyl)(phenyl)methyl)-4-(trifluoromethoxy)benzenesulfonamide;
N—((R)-((1R,2S)-2-methylcyclopropyl)(phenyl)methyl)-4-(trifluoromethoxy)benzenesulfonamide;
(R)—N-(1-(3,4-dichlorophenyl)-2-(dimethylamino)ethyl)-3-(trifluoromethoxy)benzenesulfonamide;
(R)-2-amino-N-(1-(3,4-dichlorophenyl)-2-(dimethylamino)ethyl)-4-(trifluoromethoxy)benzenesulfonamide;
(R)—N-(1-(3,4-dichlorophenyl)-2-(dimethylamino)ethyl)naphthalene-1-sulfonamide;
(R)-2-chloro-N-(1-(3,4-dichlorophenyl)-2-(dimethylamino)ethyl)-4-(trifluoromethoxy)benzenesulfonamide;
(R)—N-(1-(3,4-dichlorophenyl)-2-(dimethylamino)ethyl)naphthalene-2-sulfonamide;
(R)-3,4-dichloro-N-(1-(3,4-dichlorophenyl)-2-(dimethylamino)ethyl)benzenesulfonamide;
N-(((2R,3R)-2-(4-methoxyphenyl)-1-methylpyrrolidin-3-yl)methyl)-4-(trifluoromethoxy)benzenesulfonamide;
N-(((2R,3S)-2-(4-methoxyphenyl)-1-methylpyrrolidin-3-yl)methyl)-4-(trifluoromethoxy)benzenesulfonamide;
(R)—N-(1-hydroxy-3-(m-tolyl)propan-2-yl)-4-(trifluoromethoxy)benzenesulfonamide;
(R)—N-(1-(3,4-dichlorophenyl)-2-(dimethylamino)ethyl)-2-phenylethane-1-sulfonamide;
(R)—N-(1-(3,4-dichlorophenyl)-2-(pyrrolidin-1-yl)ethyl)-4-(trifluoromethoxy)benzenesulfonamide;
(R)—N-(1-(3,4-dichlorophenyl)-2-(dimethylamino)ethyl)-N′-methyl-4-(trifluoromethoxy)benzenesulfonimidamide;
(R)—N-(1-(3,4-dichlorophenyl)-2-(dimethylamino)ethyl)-2,2-difluorobenzo[d][1,3]dioxole-5-sulfonamide;
(R)-ethyl 3-((4-(trifluoromethoxy)phenyl)sulfonamido)-3-(3-(trifluoromethyl)phenyl)propanoate;
(R)—N-(1-(1-(4-bromophenyl)cyclopropyl)ethyl)-4-(trifluoromethoxy)benzenesulfonamide;
(R)-4-methyl-N-(thiazol-2-yl)-2-((4-(trifluoromethoxy)phenyl)sulfonamido)pentanamide;
(R)—N-(1-(3,4-dichlorophenyl)-2-(dimethylamino)ethyl)-4-isopropylbenzenesulfonamide;
(R)—N-(1-(3,4-dichlorophenyl)-2-(dimethylamino)ethyl)-4-(trifluoromethyl)benzenesulfonamide;
(R)—N-(4,4,4-trifluoro-1-(2,4,5-trifluorophenyl)butan-2-yl)-4-(trifluoromethoxy)benzenesulfonamide;
(R)—N-((6-benzyl-6-azaspiro[3.4]octan-8-yl)methyl)-4-(trifluoromethoxy)benzenesulfonamide;
(R)-4-chloro-N-(1-(3,4-dichlorophenyl)-2-(dimethylamino)ethyl)benzenesulfonamide;
(R)—N-(1-(3-cyanophenyl)-3-methylbutyl)-4-(trifluoromethoxy)benzenesulfonamide;
(R)—N-(cyclohexyl(3-(trifluoromethyl)phenyl)methyl)-4-(trifluoromethoxy)benzenesulfonamide;
(R,E)-N-(1-(3,4-dichlorophenyl)-2-(dimethylamino)ethyl)-2-phenylethene-1-sulfonamide;
(R)—N-(2-(2-hydroxypropoxy)benzyl)-4-(trifluoromethoxy)benzenesulfonamide;
(R)-3-amino-N-(1-(3,4-dichlorophenyl)-2-(dimethylamino)ethyl)-4-(trifluoromethoxy)benzenesulfonamide;
N-((1S)-(4-fluorophenyl)(tetrahydrofuran-2-yl)methyl)-4-(trifluoromethoxy)benzenesulfonamide;
(R)—N-(1-(4-chlorophenyl)-2-(piperidin-1-yl)ethyl)-4-(trifluoromethoxy)benzenesulfonamide;
N—((R)-1-((R)-1-methylpiperidin-2-yl)propan-2-yl)-4-(trifluoromethoxy)benzenesulfonamide;
N—((R)-1-((S)-1-methylpiperidin-2-yl)propan-2-yl)-4-(trifluoromethoxy)benzenesulfonamide;
(R)—N-(1-(3,4-dichlorophenyl)-2-(dimethylamino)ethyl)-4-ethylbenzenesulfonamide;
(R)—N-(1-(2,5-difluorophenyl)propyl)-4-(trifluoromethoxy)benzenesulfonamide;
methyl (R)-3-(3,5-dichlorophenyl)-3-((4-(trifluoromethoxy)phenyl)sulfonamido)propanoate;
(R)—N-((3-chlorophenyl)(cyclopentyl)methyl)-4-(trifluoromethoxy)benzenesulfonamide;
(R)—N-(1-(benzo[d][1,3]dioxol-5-yl)butyl)-4-(trifluoromethoxy)benzenesulfonamide;
methyl (R)-3-(3-fluoro-4-hydroxyphenyl)-2-((4-(trifluoromethoxy)phenyl)sulfonamido)propanoate;
methyl (R)-3-(3-bromo-4-methoxyphenyl)-3-((4-(trifluoromethoxy)phenyl)sulfonamido)propanoate;
tert-butyl (R)-2-((4-(trifluoromethoxy)phenyl)sulfonamido)hexanoate;
(R)—N-(cyclopropyl(4-fluoro-3-(trifluoromethyl)phenyl)methyl)-4-(trifluoromethoxy)benzenesulfonamide;
(R)—N-(1-(3,4-dichlorophenyl)-2-hydroxyethyl)-4-(trifluoromethoxy)benzenesulfonamide;
(R)—N-(1-(3,4-dichlorophenyl)-2-(dimethylamino)ethyl)-3-methylbenzenesulfonamide;
methyl (R)-2-(2-bromophenyl)-2-((4-(trifluoromethoxy)phenyl)sulfonamido)acetate;
tert-butyl (R)-4,4-difluoro-2-(((4-(trifluoromethoxy)phenyl)sulfonamido)methyl)pyrrolidine-1-carboxylate;
(R)—N-(1-(3-chlorophenyl)-2-(dimethylamino)ethyl)-4-(trifluoromethoxy)benzenesulfonamide;
(R)—N-(2-morpholino-2-(4-(trifluoromethyl)phenyl)ethyl)-4-(trifluoromethoxy)benzenesulfonamide;
(R)—N-(2-(dimethylamino)-1-(p-tolyl)ethyl)-4-(trifluoromethoxy)benzenesulfonamide;
(R)—N-(1-(3,4-dichlorophenyl)-2-(dimethylamino)ethyl)-4-(difluoromethoxy)benzenesulfonamide;
(R)—N-(2-(furan-2-yl)-2-(4-methylpiperidin-1-yl)ethyl)-4-(trifluoromethoxy)benzenesulfonamide;
ethyl (R)-3-(2-chlorophenyl)-2-((4-(trifluoromethoxy)phenyl)sulfonamido)propanoate;
(R)—N-(2-(tert-butoxy)benzyl)-4-(trifluoromethoxy)benzenesulfonamide;
(R)-3-chloro-N-(1-(3,4-dichlorophenyl)-2-(dimethylamino)ethyl)benzenesulfonamide;
(R)—N-(2-(dimethylamino)-1-(3-(trifluoromethyl)phenyl)ethyl)-4-(trifluoromethoxy)benzenesulfonamide;
(R,R)—N-(1-(3,4-dichlorophenyl)-2-(dimethylamino)ethyl)-4-(trifluoromethoxy)benzenesulfonimidamide;
(R,S)—N-(1-(3,4-dichlorophenyl)-2-(dimethylamino)ethyl)-4-(trifluoromethoxy)benzenesulfonimidamide;
(R)—N-(1-(5-fluoro-2-(4-hydroxypiperidin-1-yl)phenyl)ethyl)-4-(trifluoromethoxy)benzenesulfonamide;
(R)—N-(2-(dimethylamino)-1-(m-tolyl)ethyl)-4-(trifluoromethoxy)benzenesulfonamide;
ethyl (R)-3-(3-bromophenyl)-3-((4-(trifluoromethoxy)phenyl)sulfonamido)propanoate;
(R)—N-(1-(3,4-dichlorophenyl)-2-(dimethylamino)ethyl)-2-methylbenzenesulfonamide;
(R)-4-chloro-N-(1-(3,4-dichlorophenyl)-2-(dimethylamino)ethyl)-3-fluorobenzenesulfonamide;
ethyl (R)-3-(3-chlorophenyl)-2-((4-(trifluoromethoxy)phenyl)sulfonamido)propanoate;
(R)—N-(1-(2-bromo-4-fluorophenyl)-2-cyclopropylethyl)-4-(trifluoromethoxy)benzenesulfonamide;
ethyl (R)-2-(3-bromo-4-methoxyphenyl)-2-((4-(trifluoromethoxy)phenyl)sulfonamido)acetate;
methyl (R)-3-(2-hydroxyphenyl)-2-((4-(trifluoromethoxy)phenyl)sulfonamido)propanoate;
(R)—N-(1-(3,4-dichlorophenyl)-3-methoxypropyl)-4-(trifluoromethoxy)benzenesulfonamide;
(R)—N-(1-(3,4-dichlorophenyl)-2-(dimethylamino)ethyl)-4-ethoxybenzenesulfonamide;
(R)—N-(1-(4-chlorophenyl)-2-(pyrrolidin-1-yl)ethyl)-4-(trifluoromethoxy)benzenesulfonamide;
(R)—N-(1-(3,4-dichlorophenyl)-2-(dimethylamino)ethyl)-4-methylbenzenesulfonamide;
(R)—N′-(1-(3,4-dichlorophenyl)-2-(dimethylamino)ethyl)-N-methyl-4-(trifluoromethoxy)benzenesulfonimidamide;
(R)—N-(1-(3,4-dichlorophenyl)-2-(dimethylamino)ethyl)-3-(trifluoromethyl)benzenesulfonamide;
(R)—N-cyclohexyl-4-(N-(1-(3,4-dichlorophenyl)-2-(dimethylamino)ethyl)sulfamoyl)benzamide;
(R)—N-(1-(3,4-dichlorophenyl)-2-morpholinoethyl)-4-(trifluoromethoxy)benzenesulfonamide;
(R)—N-cycloheptyl-N-methyl-2-((4-(trifluoromethoxy)phenyl)sulfonamido)pentanamide;
(R)—N-(4,4,4-trifluoro-3-phenylbutyl)-4-(trifluoromethoxy)benzenesulfonamide;
(R)—N-(1-(5-ethylfuran-2-yl)-2,2-dimethylpropyl)-4-(trifluoromethoxy)benzenesulfonamide;
(R)—N-(2-(4-ethylphenyl)-2-(pyrrolidin-1-yl)ethyl)-4-(trifluoromethoxy)benzenesulfonamide;
(R)—N-(1-(4-chlorophenyl)-2-(dimethylamino)ethyl)-4-(trifluoromethoxy)benzenesulfonamide;
methyl (R)-3-(3,4-dichlorophenyl)-2-((4-(trifluoromethoxy)phenyl)sulfonamido)propanoate;
(R)—N-(1-(3,4-dichlorophenyl)-2-(dimethylamino)ethyl)cyclohexanesulfonamide;
ethyl (R)-3-(2,5-dichlorothiophen-3-yl)-2-((4-(trifluoromethoxy)phenyl)sulfonamido)propanoate;
tert-butyl ((4-(trifluoromethoxy)phenyl)sulfonyl)-D-isoleucinate;
tert-butyl ((4-(trifluoromethoxy)phenyl)sulfonyl)-D-alloisoleucinate
(R)-4-chloro-N-(1-(3,4-dichlorophenyl)-2-(dimethylamino)ethyl)-2-fluorobenzenesulfonamide;
(R)—N-(1-(3-bromophenyl)-4,4,4-trifluorobutan-2-yl)-4-(trifluoromethoxy)benzenesulfonamide;
(R)—N-(2-(dimethylamino)-1-(o-tolyl)ethyl)-4-(trifluoromethoxy)benzenesulfonamide;
methyl (R)-3-cyclohexyl-3-((4-(trifluoromethoxy)phenyl)sulfonamido)propanoate;
methyl (R)-4-phenyl-3-((4-(trifluoromethoxy)phenyl)sulfonamido)butanoate;
(R)—N-(1-(3,4-dichlorophenyl)-2-(dimethylamino)ethyl)-4-fluorobenzenesulfonamide;
(R)-3-(4-isopropylphenyl)-3-((4-(trifluoromethoxy)phenyl)sulfonamido)propanoic acid;
methyl (R)-2-phenyl-2-((4-(trifluoromethoxy)phenyl)sulfonamido)butanoate;
(R)—N-(1-(3,4-dichlorophenyl)-2-(dimethylamino)ethyl)-3-fluorobenzenesulfonamide;
ethyl (R)-4-methyl-2-((4-(trifluoromethoxy)phenyl)sulfonamido)hexanoate;
(R)—N-(3-(1-cyanopropoxy)benzyl)-4-(trifluoromethoxy)benzenesulfonamide;
ethyl (R)-3-(4-methoxyphenyl)-2-((4-(trifluoromethoxy)phenyl)sulfonamido)propanoate;
ethyl (R)-3-(4-isopropoxy-3-methoxyphenyl)-3-((4-(trifluoromethoxy)phenyl)sulfonamido)propanoate;
(R)—N-((4-chlorophenyl)(cyclopentyl)methyl)-4-(trifluoromethoxy)benzenesulfonamide;
ethyl (R)-3-(4-ethoxyphenyl)-3-((4-(trifluoromethoxy)phenyl)sulfonamido)propanoate;
(R)—N-(1-(3,4-dichlorophenyl)-2-(dimethylamino)ethyl)-3,5-difluorobenzenesulfonamide;
(R)—N-(1-(2-methoxy-5-methylphenyl)-3-methylbutan-2-yl)-4-(trifluoromethoxy)benzenesulfonamide;
N-(2-(((1R,2R)-2-methylcyclohexyl)oxy)ethyl)-4-(trifluoromethoxy)benzenesulfonamide;
N-(2-(((1R,2S)-2-methylcyclohexyl)oxy)ethyl)-4-(trifluoromethoxy)benzenesulfonamide;
N-(((1R,2R)-2-phenylcyclohexyl)methyl)-4-(trifluoromethoxy)benzenesulfonamide;
N-(((1R,2S)-2-phenylcyclohexyl)methyl)-4-(trifluoromethoxy)benzenesulfonamide;
N-(((1R,3R)-1-(dimethylamino)-3-methylcyclohexyl)methyl)-4-(trifluoromethoxy)benzenesulfonamide;
N-(((1R,3S)-1-(dimethylamino)-3-methylcyclohexyl)methyl)-4-(trifluoromethoxy)benzenesulfonamide;
N-((1R,2R)-2-(methylsulfonyl)-1-(p-tolyl)propyl)-4-(trifluoromethoxy)benzenesulfonamide;
N-((1R,2S)-2-(methylsulfonyl)-1-(p-tolyl)propyl)-4-(trifluoromethoxy)benzenesulfonamide;
N-((1R,2R)-1-(2-chlorophenyl)-1-methoxypropan-2-yl)-4-(trifluoromethoxy)benzenesulfonamide;
N-((1R,2S)-1-(2-chlorophenyl)-1-methoxypropan-2-yl)-4-(trifluoromethoxy)benzenesulfonamide;
ethyl (R)-3-((R)-tetrahydrofuran-3-yl)-2-((4-(trifluoromethoxy)phenyl)sulfonamido)propanoate;
ethyl (R)-3-((S)-tetrahydrofuran-3-yl)-2-((4-(trifluoromethoxy)phenyl)sulfonamido)propanoate;
ethyl (R)-3-((R)-cyclohex-3-en-1-yl)-2-((4-(trifluoromethoxy)phenyl)sulfonamido)propanoate;
ethyl (R)-3-((S)-cyclohex-3-en-1-yl)-2-((4-(trifluoromethoxy)phenyl)sulfonamido)propanoate;
N—((R)-(4-fluorophenyl)((R)-tetrahydrofuran-2-yl)methyl)-4-(trifluoromethoxy)benzenesulfonamide;
N—((R)-(4-fluorophenyl)((S)-tetrahydrofuran-2-yl)methyl)-4-(trifluoromethoxy)benzenesulfonamide;
(R)-4-cyclopropoxy-N-(1-(3,4-dichlorophenyl)-2-(dimethylamino)ethyl)benzenesulfonamide;
(R)—N-(1-(3-chloro-4-fluorophenyl)-2-(dimethylamino)ethyl)-4-(trifluoromethoxy)benzenesulfonamide;
(R)—N-(1-(4-chloro-3-fluorophenyl)-2-(dimethylamino)ethyl)-4-(trifluoromethoxy)benzenesulfonamide;
(R)-4-(N-(1-(3,4-dichlorophenyl)-2-(dimethylamino)ethyl)sulfamoyl)-N-phenylbenzamide;
(R)—N-(1-(3,4-dichlorophenyl)-2-(dimethylamino)ethyl)-4-(phenylamino)benzenesulfonamide;
(R)—N-(1-(4-chlorophenyl)-2-(dimethylamino)ethyl)-4-(4-(trifluoromethyl)phenoxy)benzenesulfonamide;
(R)—N-(2-(dimethylamino)-1-(3-(trifluoromethyl)phenyl)ethyl)-3-nitro-4-(trifluoromethoxy)benzenesulfonamide;
(R)—N-(1-(4-chlorophenyl)-2-(dimethylamino)ethyl)-3-nitro-4-(trifluoromethoxy)benzenesulfonamide;
(R)-4-(cyclopropylmethoxy)-N-(1-(3,4-dichlorophenyl)-2-(dimethylamino)ethyl)benzenesulfonamide;
(R)—N-(1-(3,4-dichlorophenyl)-2-(dimethylamino)ethyl)-4-isopropoxybenzenesulfonamide;
(R)-4-(cyclohexyloxy)-N-(1-(3,4-dichlorophenyl)-2-(dimethylamino)ethyl)benzenesulfonamide;
(R)—N-(1-(3,4-dichlorophenyl)-2-(dimethylamino)ethyl)-4-(pyridin-2-yloxy)benzenesulfonamide;
(R)-4-butyl-N-(1-(3,4-dichlorophenyl)-2-(dimethylamino)ethyl)benzenesulfonamide;
(R)—N-(1-(3,4-dichlorophenyl)-2-(dimethylamino)ethyl)-4-(3,6-dihydro-2H-pyran-4-yl)benzenesulfonamide;
(R)—N-(2-(dimethylamino)-1-(4-(trifluoromethyl)phenyl)ethyl)-4-(trifluoromethoxy)benzenesulfonamide;
(R)—N-(1-(3,4-dichlorophenyl)-2-(dimethylamino)ethyl)-4-(3-fluorophenoxy)benzenesulfonamide;
(R)—N-(1-(3,4-dichlorophenyl)-2-(dimethylamino)ethyl)-4-(4-fluorophenoxy)benzenesulfonamide;
(R)-methyl 5-(N-(1-(3,4-dichlorophenyl)-2-(dimethylamino)ethyl)sulfamoyl)-2-(trifluoromethoxy)benzoate;
(R)—N-(1-(3,4-dichlorophenyl)-2-(4-methylpiperazin-1-yl)ethyl)-4-(4-(trifluoromethyl)phenoxy)benzenesulfonamide;
(R)—N-(1-(4-chlorophenyl)-2-(4-methylpiperazin-1-yl)ethyl)-4-phenoxybenzenesulfonamide;
(R)—N-(1-(4-chlorophenyl)-2-(4-methylpiperazin-1-yl)ethyl)-4-(4-(trifluoromethyl)phenoxy)benzenesulfonamide;
(R)—N-(1-(3,4-dichlorophenyl)-2-(4-methylpiperazin-1-yl)ethyl)-4-phenoxybenzenesulfonamide;
(R)—N-(2-(dimethylamino)-1-(3-(trifluoromethyl)phenyl)ethyl)-4-(4-(trifluoromethyl)phenoxy)benzenesulfonamide;
(R)—N-(1-(3,4-dichlorophenyl)-2-(dimethylamino)ethyl)-6-(trifluoromethoxy)pyridine-3-sulfonamide;
(R)—N-(1-(3,4-dichlorophenyl)-2-(dimethylamino)ethyl)-3-nitro-4-(trifluoromethoxy)benzenesulfonamide;
(R)—N-(1-(3,4-dichlorophenyl)-2-(dimethylamino)ethyl)-4-(pyridin-2-yl)benzenesulfonamide;
(R)—N-(1-(3,4-dichlorophenyl)-2-(dimethylamino)ethyl)-4-nitro-3-(trifluoromethoxy)benzenesulfonamide;
(R)—N-(2-amino-1-(3,4-dichlorophenyl)ethyl)-4-(trifluoromethoxy)benzenesulfonamide hydrochloride;
(R)-tert-butyl (2-(3,4-dichlorophenyl)-2-((4-(trifluoromethoxy)phenyl)sulfonamido)ethyl)carbamate;
(R)—N-((2,2-dimethyl-1-phenylcyclopropyl)methyl)-4-(trifluoromethoxy)benzenesulfonamide;
(R)—N-(1-(3,4-dichlorophenyl)-2-(dimethylamino)ethyl)-2-nitro-3-(trifluoromethoxy)benzenesulfonamide;
4-(trifluoromethoxy)-N-((1R,3R)-3,4,4-trimethylcyclohexyl)benzenesulfonamide;
4-(trifluoromethoxy)-N-((1R,3S)-3,4,4-trimethylcyclohexyl)benzenesulfonamide;
(R)—N-(1-(3,4-dichlorophenyl)-2-(dimethylamino)ethyl)-4-(2-(trifluoromethyl)phenoxy)benzenesulfonamide;
(R)—N-(1-(3,4-dichlorophenyl)-2-(dimethylamino)ethyl)-4-(tetrahydro-2H-pyran-2-yl)benzenesulfonamide;
(R)-3-cyano-N-(1-(3,4-dichlorophenyl)-2-(dimethylamino)ethyl)-4-(trifluoromethoxy)benzenesulfonamide;
(R)—N-(1-(3,4-dichlorophenyl)-2-(4-propoxypiperidin-1-yl)ethyl)-4-(trifluoromethoxy)benzenesulfonamide;
(R)—N-(1-(3,4-dichlorophenyl)-2-(methylamino)ethyl)-4-(trifluoromethoxy)benzenesulfonamide;
(R)—N-(1-(3,4-dichlorophenyl)-3-(dimethylamino)propan-2-yl)-4-(trifluoromethoxy)benzenesulfonamide;
(R)-4-cyclobutoxy-N-(1-(3,4-dichlorophenyl)-2-(dimethylamino)ethyl)benzenesulfonamide;
(R)-3-(3,4-dichlorophenyl)-N,N-dimethyl-2-((4-(trifluoromethoxy)phenyl)sulfonamido)propenamide;
(R)—N-(1-(3,4-dichlorophenyl)-2-(dimethylamino)ethyl)-N-methyl-4-(trifluoromethoxy)benzenesulfonamide;
(R)-4-(cyclopentylamino)-N-(1-(3,4-dichlorophenyl)-2-(dimethylamino)ethyl)benzenesulfonamide;
(R)-4-(cyclobutylamino)-N-(1-(3,4-dichlorophenyl)-2-(dimethylamino)ethyl)benzenesulfonamide;
(R)—N-((3-(3,4-dichlorophenyl)pyrrolidin-3-yl)methyl)-4-(trifluoromethoxy)benzenesulfonamide;
(R)-2-(3,4-dichlorophenyl)-N,N-dimethyl-2-((4-(trifluoromethoxy)phenyl)sulfonamido)acetamide;
(R)—N-(1-(3,4-dichlorophenyl)-2-((4-methoxybenzyl)amino)ethyl)-4-(trifluoromethoxy)benzenesulfonamide;
(R)—N-(1-(4-chlorophenyl)-2-(piperazin-1-yl)ethyl)-4-(trifluoromethoxy)benzenesulfonamide;
(R)—N-(1-(4-chloro-3-fluorophenyl)-2-(piperazin-1-yl)ethyl)-4-(trifluoromethoxy)benzenesulfonamide;
(R)—N-(1-(4-chlorophenyl)-2-(piperazin-1-yl)ethyl)-6-(trifluoromethoxy)pyridine-3-sulfonamide;
N—((R)-2-((1R,4R)-2,5-diazabicyclo[2.2.1]heptan-2-yl)-1-(3,4-dichlorophenyl)ethyl)-4-(trifluoromethoxy)benzenesulfonamide;
N—((R)-2-((1S,4S)-2,5-diazabicyclo[2.2.1]heptan-2-yl)-1-(3,4-dichlorophenyl)ethyl)-4-(trifluoromethoxy)benzenesulfonamide;
(R)—N-((2-phenylpyrrolidin-2-yl)methyl)-4-(trifluoromethoxy)benzenesulfonamide;
(R)—N-(1-(4-fluorophenyl)-2-(piperazin-1-yl)ethyl)-4-(trifluoromethoxy)benzenesulfonamide;
(R)—N-(2-(4-fluorophenyl)-2-(piperazin-1-yl)ethyl)-4-(trifluoromethoxy)benzenesulfonamide;
(R)—N-(1-(3,4-dichlorophenyl)-2-(dimethylamino)ethyl)-3-nitro-4-(trifluoromethyl)benzenesulfonamide;
(R)—N-(1-(3,4-dichlorophenyl)-2-(dimethylamino)ethyl)-4-propoxybenzenesulfonamide;
(R)-4-(cyclohexylamino)-N-(1-(3,4-dichlorophenyl)-2-(dimethylamino)ethyl)benzenesulfonamide; and
(R)—N-(1-(3,4-dichlorophenyl)-2-(4-methylpiperazin-1-yl)ethyl)-4-(4-fluorophenoxy)benzenesulfonamide;
or a salt, solvate, isotopologue, or tautomer thereof.

Embodiment 29 provides the compound of Embodiment 1, which is selected from the group consisting of
(S)—N-(1-(3,4-dichlorophenyl)-2-(dimethylamino)ethyl)-3-nitro-4-(trifluoromethoxy)benzenesulfonamide;
(S)—N-(1-hydroxy-5-methylhexan-3-yl)-4-(trifluoromethoxy)benzenesulfonamide;
(S)—N-(1-(3,4-dichlorophenyl)-2-(dimethylamino)ethyl)-4-pentylbenzenesulfonamide;
(S)—N-(cyclohexyl(3,5-dichlorophenyl)methyl)-4-(trifluoromethoxy)benzenesulfonamide;
(S)—N-(1-(3,4-dichlorophenyl)-2-(dimethylamino)ethyl)-4-(4-(trifluoromethyl)phenoxy)benzenesulfonamide;
(S)—N-(1-(3,4-dichlorophenyl)-2-(dimethylamino)ethyl)-4-phenoxybenzenesulfonamide;
(S)—N-(1-(3,4-dichlorophenyl)-2-(dimethylamino)ethyl)-[1,1′-biphenyl]-4-sulfonamide;
(S)—N-(1-(4-chlorophenyl)-2-(dimethylamino)ethyl)-4-phenoxybenzenesulfonamide;
(S)—N-(2-(dimethylamino)-1-(3-(trifluoromethyl)phenyl)ethyl)-4-phenoxybenzenesulfonamide;
(S)-3-chloro-N-(1-(3,4-dichlorophenyl)-2-(dimethylamino)ethyl)-4-(trifluoromethoxy)benzenesulfonamide;
(S)-4-(cyclopentyloxy)-N-(1-(3,4-dichlorophenyl)-2-(dimethylamino)ethyl)benzenesulfonamide;
(S)—N-(1-(3,4-dichlorophenyl)-2-(dimethylamino)ethyl)-4-propylbenzenesulfonamide;
(S)—N-(1-(3,4-dichlorophenyl)-2-(4-methylpiperazin-1-yl)ethyl)-4-(trifluoromethoxy)benzenesulfonamide;
(S)—N-(1-(3,4-dichlorophenyl)-2-(dimethylamino)ethyl)-[1,1′-biphenyl]-3-sulfonamide;
(S)-3-chloro-N-(1-(4-chlorophenyl)-2-(dimethylamino)ethyl)-4-(trifluoromethoxy)benzenesulfonamide;
(S)-3-chloro-N-(2-(dimethylamino)-1-(3-(trifluoromethyl)phenyl)ethyl)-4-(trifluoromethoxy)benzenesulfonamide;
(S)—N-(1-(3-bromo-5-fluorophenyl)-2-cyclopropylethyl)-4-(trifluoromethoxy)benzenesulfonamide;
(S)—N-((1-(2,4-difluorophenyl)-2,2-dimethylcyclopropyl)methyl)-4-(trifluoromethoxy)benzenesulfonamide;
(S)—N-(1-(3,4-dichlorophenyl)-2-(dimethylamino)ethyl)-4-(trifluoromethoxy)benzenesulfonamide;
(S)—N-(2-(dimethylamino)-1-(3-(trifluoromethyl)phenyl)ethyl)-4-(4-(trifluoromethyl)phenoxy)benzenesulfonamide;
(S)—N-(2-(isopropyl(methyl)amino)benzyl)-4-(trifluoromethoxy)benzenesulfonamide;
(S)-4-(trifluoromethoxy)-N-(1-(4-(trifluoromethyl)phenyl)pent-4-en-1-yl)benzenesulfonamide;
N—((S)-((1S,2S)-2-methylcyclopropyl)(phenyl)methyl)-4-(trifluoromethoxy)benzenesulfonamide;
N—((S)-((1S,2R)-2-methylcyclopropyl)(phenyl)methyl)-4-(trifluoromethoxy)benzenesulfonamide;
(S)—N-(1-(3,4-dichlorophenyl)-2-(dimethylamino)ethyl)-3-(trifluoromethoxy)benzenesulfonamide;
(S)-2-amino-N-(1-(3,4-dichlorophenyl)-2-(dimethylamino)ethyl)-4-(trifluoromethoxy)benzenesulfonamide;
(S)—N-(1-(3,4-dichlorophenyl)-2-(dimethylamino)ethyl)naphthalene-1-sulfonamide;
(S)-2-chloro-N-(1-(3,4-dichlorophenyl)-2-(dimethylamino)ethyl)-4-(trifluoromethoxy)benzenesulfonamide;
(S)—N-(1-(3,4-dichlorophenyl)-2-(dimethylamino)ethyl)naphthalene-2-sulfonamide;
(S)-3,4-dichloro-N-(1-(3,4-dichlorophenyl)-2-(dimethylamino)ethyl)benzenesulfonamide;
N-(((2S,3S)-2-(4-methoxyphenyl)-1-methylpyrrolidin-3-yl)methyl)-4-(trifluoromethoxy)benzenesulfonamide;
N-(((2S,3R)-2-(4-methoxyphenyl)-1-methylpyrrolidin-3-yl)methyl)-4-(trifluoromethoxy)benzenesulfonamide;
(S)—N-(1-hydroxy-3-(m-tolyl)propan-2-yl)-4-(trifluoromethoxy)benzenesulfonamide;
(S)—N-(1-(3,4-dichlorophenyl)-2-(dimethylamino)ethyl)-2-phenylethane-1-sulfonamide;
(S)—N-(1-(3,4-dichlorophenyl)-2-(pyrrolidin-1-yl)ethyl)-4-(trifluoromethoxy)benzenesulfonamide;
(S)—N-(1-(3,4-dichlorophenyl)-2-(dimethylamino)ethyl)-N′-methyl-4-(trifluoromethoxy)benzenesulfonimidamide;
(S)—N-(1-(3,4-dichlorophenyl)-2-(dimethylamino)ethyl)-2,2-difluorobenzo[d][1,3]dioxole-5-sulfonamide;
(S)-ethyl 3-((4-(trifluoromethoxy)phenyl)sulfonamido)-3-(3-(trifluoromethyl)phenyl)propanoate;
(S)—N-(1-(1-(4-bromophenyl)cyclopropyl)ethyl)-4-(trifluoromethoxy)benzenesulfonamide;
(S)-4-methyl-N-(thiazol-2-yl)-2-((4-(trifluoromethoxy)phenyl)sulfonamido)pentanamide;
(S)—N-(1-(3,4-dichlorophenyl)-2-(dimethylamino)ethyl)-4-isopropylbenzenesulfonamide;
(S)—N-(1-(3,4-dichlorophenyl)-2-(dimethylamino)ethyl)-4-(trifluoromethyl)benzenesulfonamide;
(S)—N-(4,4,4-trifluoro-1-(2,4,5-trifluorophenyl)butan-2-yl)-4-(trifluoromethoxy)benzenesulfonamide;
(S)—N-((6-benzyl-6-azaspiro[3.4]octan-8-yl)methyl)-4-(trifluoromethoxy)benzenesulfonamide;
(S)-4-chloro-N-(1-(3,4-dichlorophenyl)-2-(dimethylamino)ethyl)benzenesulfonamide;
(S)—N-(1-(3-cyanophenyl)-3-methylbutyl)-4-(trifluoromethoxy)benzenesulfonamide;
(S)—N-(cyclohexyl(3-(trifluoromethyl)phenyl)methyl)-4-(trifluoromethoxy)benzenesulfonamide;
(S,E)-N-(1-(3,4-dichlorophenyl)-2-(dimethylamino)ethyl)-2-phenylethene-1-sulfonamide;
(S)—N-(2-(2-hydroxypropoxy)benzyl)-4-(trifluoromethoxy)benzenesulfonamide;
(S)-3-amino-N-(1-(3,4-dichlorophenyl)-2-(dimethylamino)ethyl)-4-(trifluoromethoxy)benzenesulfonamide;
N-((1S)-(4-fluorophenyl)(tetrahydrofuran-2-yl)methyl)-4-(trifluoromethoxy)benzenesulfonamide;
(S)—N-(1-(4-chlorophenyl)-2-(piperidin-1-yl)ethyl)-4-(trifluoromethoxy)benzenesulfonamide;
N—((S)-1-((S)-1-methylpiperidin-2-yl)propan-2-yl)-4-(trifluoromethoxy)benzenesulfonamide;
N—((S)-1-((R)-1-methylpiperidin-2-yl)propan-2-yl)-4-(trifluoromethoxy)benzenesulfonamide;
(S)—N-(1-(3,4-dichlorophenyl)-2-(dimethylamino)ethyl)-4-ethylbenzenesulfonamide;
(S)—N-(1-(2,5-difluorophenyl)propyl)-4-(trifluoromethoxy)benzenesulfonamide;
methyl (S)-3-(3,5-dichlorophenyl)-3-((4-(trifluoromethoxy)phenyl)sulfonamido)propanoate;
(S)—N-((3-chlorophenyl)(cyclopentyl)methyl)-4-(trifluoromethoxy)benzenesulfonamide;
(S)—N-(1-(benzo[d][1,3]dioxol-5-yl)butyl)-4-(trifluoromethoxy)benzenesulfonamide;
methyl (S)-3-(3-fluoro-4-hydroxyphenyl)-2-((4-(trifluoromethoxy)phenyl)sulfonamido)propanoate;
methyl (S)-3-(3-bromo-4-methoxyphenyl)-3-((4-(trifluoromethoxy)phenyl)sulfonamido)propanoate;
tert-butyl (S)-2-((4-(trifluoromethoxy)phenyl)sulfonamido)hexanoate;
(S)—N-(cyclopropyl(4-fluoro-3-(trifluoromethyl)phenyl)methyl)-4-(trifluoromethoxy)benzenesulfonamide;
(S)—N-(1-(3,4-dichlorophenyl)-2-hydroxyethyl)-4-(trifluoromethoxy)benzenesulfonamide;
(S)—N-(1-(3,4-dichlorophenyl)-2-(dimethylamino)ethyl)-3-methylbenzenesulfonamide;
methyl (S)-2-(2-bromophenyl)-2-((4-(trifluoromethoxy)phenyl)sulfonamido)acetate;
tert-butyl (S)-4,4-difluoro-2-(((4-(trifluoromethoxy)phenyl)sulfonamido)methyl)pyrrolidine-1-carboxylate;
(S)—N-(1-(3-chlorophenyl)-2-(dimethylamino)ethyl)-4-(trifluoromethoxy)benzenesulfonamide;
(S)—N-(2-morpholino-2-(4-(trifluoromethyl)phenyl)ethyl)-4-(trifluoromethoxy)benzenesulfonamide;
(S)—N-(2-(dimethylamino)-1-(p-tolyl)ethyl)-4-(trifluoromethoxy)benzenesulfonamide;
(S)—N-(1-(3,4-dichlorophenyl)-2-(dimethylamino)ethyl)-4-(difluoromethoxy)benzenesulfonamide;
(S)—N-(2-(furan-2-yl)-2-(4-methylpiperidin-1-yl)ethyl)-4-(trifluoromethoxy)benzenesulfonamide;
ethyl (S)-3-(2-chlorophenyl)-2-((4-(trifluoromethoxy)phenyl)sulfonamido)propanoate;
(S)—N-(2-(tert-butoxy)benzyl)-4-(trifluoromethoxy)benzenesulfonamide;
(S)-3-chloro-N-(1-(3,4-dichlorophenyl)-2-(dimethylamino)ethyl)benzenesulfonamide;
(S)—N-(2-(dimethylamino)-1-(3-(trifluoromethyl)phenyl)ethyl)-4-(trifluoromethoxy)benzenesulfonamide;
(S,S)—N-(1-(3,4-dichlorophenyl)-2-(dimethylamino)ethyl)-4-(trifluoromethoxy)benzenesulfonimidamide;
(S,R)—N-(1-(3,4-dichlorophenyl)-2-(dimethylamino)ethyl)-4-(trifluoromethoxy)benzenesulfonimidamide;
(S)—N-(1-(5-fluoro-2-(4-hydroxypiperidin-1-yl)phenyl)ethyl)-4-(trifluoromethoxy)benzenesulfonamide;
(S)—N-(2-(dimethylamino)-1-(m-tolyl)ethyl)-4-(trifluoromethoxy)benzenesulfonamide;
ethyl (S)-3-(3-bromophenyl)-3-((4-(trifluoromethoxy)phenyl)sulfonamido)propanoate;
(S)—N-(1-(3,4-dichlorophenyl)-2-(dimethylamino)ethyl)-2-methylbenzenesulfonamide;
(S)-4-chloro-N-(1-(3,4-dichlorophenyl)-2-(dimethylamino)ethyl)-3-fluorobenzenesulfonamide;
ethyl (S)-3-(3-chlorophenyl)-2-((4-(trifluoromethoxy)phenyl)sulfonamido)propanoate;
(S)—N-(1-(2-bromo-4-fluorophenyl)-2-cyclopropylethyl)-4-(trifluoromethoxy)benzenesulfonamide;
ethyl (S)-2-(3-bromo-4-methoxyphenyl)-2-((4-(trifluoromethoxy)phenyl)sulfonamido)acetate;
methyl (S)-3-(2-hydroxyphenyl)-2-((4-(trifluoromethoxy)phenyl)sulfonamido)propanoate;
(S)—N-(1-(3,4-dichlorophenyl)-3-methoxypropyl)-4-(trifluoromethoxy)benzenesulfonamide;
(S)—N-(1-(3,4-dichlorophenyl)-2-(dimethylamino)ethyl)-4-ethoxybenzenesulfonamide;
(S)—N-(1-(4-chlorophenyl)-2-(pyrrolidin-1-yl)ethyl)-4-(trifluoromethoxy)benzenesulfonamide;
(S)—N-(1-(3,4-dichlorophenyl)-2-(dimethylamino)ethyl)-4-methylbenzenesulfonamide;
(S)—N′-(1-(3,4-dichlorophenyl)-2-(dimethylamino)ethyl)-N-methyl-4-(trifluoromethoxy)benzenesulfonimidamide;
(S)—N-(1-(3,4-dichlorophenyl)-2-(dimethylamino)ethyl)-3-(trifluoromethyl)benzenesulfonamide;
(S)—N-cyclohexyl-4-(N-(1-(3,4-dichlorophenyl)-2-(dimethylamino)ethyl)sulfamoyl)benzamide;
(S)—N-(1-(3,4-dichlorophenyl)-2-morpholinoethyl)-4-(trifluoromethoxy)benzenesulfonamide;
(S)—N-cycloheptyl-N-methyl-2-((4-(trifluoromethoxy)phenyl)sulfonamido)pentanamide;
(S)—N-(4,4,4-trifluoro-3-phenylbutyl)-4-(trifluoromethoxy)benzenesulfonamide;
(S)—N-(1-(5-ethylfuran-2-yl)-2,2-dimethylpropyl)-4-(trifluoromethoxy)benzenesulfonamide;
(S)—N-(2-(4-ethylphenyl)-2-(pyrrolidin-1-yl)ethyl)-4-(trifluoromethoxy)benzenesulfonamide;
(S)—N-(1-(4-chlorophenyl)-2-(dimethylamino)ethyl)-4-(trifluoromethoxy)benzenesulfonamide;
methyl (S)-3-(3,4-dichlorophenyl)-2-((4-(trifluoromethoxy)phenyl)sulfonamido)propanoate;
(S)—N-(1-(3,4-dichlorophenyl)-2-(dimethylamino)ethyl)cyclohexanesulfonamide;
ethyl (S)-3-(2,5-dichlorothiophen-3-yl)-2-((4-(trifluoromethoxy)phenyl)sulfonamido)propanoate;
tert-butyl ((4-(trifluoromethoxy)phenyl)sulfonyl)-L-isoleucinate;
tert-butyl ((4-(trifluoromethoxy)phenyl)sulfonyl)-L-alloisoleucinate
(S)-4-chloro-N-(1-(3,4-dichlorophenyl)-2-(dimethylamino)ethyl)-2-fluorobenzenesulfonamide;
(S)—N-(1-(3-bromophenyl)-4,4,4-trifluorobutan-2-yl)-4-(trifluoromethoxy)benzenesulfonamide;
(S)—N-(2-(dimethylamino)-1-(o-tolyl)ethyl)-4-(trifluoromethoxy)benzenesulfonamide;
methyl (S)-3-cyclohexyl-3-((4-(trifluoromethoxy)phenyl)sulfonamido)propanoate;
methyl (S)-4-phenyl-3-((4-(trifluoromethoxy)phenyl)sulfonamido)butanoate;
(S)—N-(1-(3,4-dichlorophenyl)-2-(dimethylamino)ethyl)-4-fluorobenzenesulfonamide;
(S)-3-(4-isopropylphenyl)-3-((4-(trifluoromethoxy)phenyl)sulfonamido)propanoic acid;
methyl (S)-2-phenyl-2-((4-(trifluoromethoxy)phenyl)sulfonamido)butanoate;
(S)—N-(1-(3,4-dichlorophenyl)-2-(dimethylamino)ethyl)-3-fluorobenzenesulfonamide;
ethyl (S)-4-methyl-2-((4-(trifluoromethoxy)phenyl)sulfonamido)hexanoate;
(S)—N-(3-(1-cyanopropoxy)benzyl)-4-(trifluoromethoxy)benzenesulfonamide;
ethyl (S)-3-(4-methoxyphenyl)-2-((4-(trifluoromethoxy)phenyl)sulfonamido)propanoate;
ethyl (S)-3-(4-isopropoxy-3-methoxyphenyl)-3-((4-(trifluoromethoxy)phenyl)sulfonamido)propanoate;
(S)—N-((4-chlorophenyl)(cyclopentyl)methyl)-4-(trifluoromethoxy)benzenesulfonamide;
ethyl (S)-3-(4-ethoxyphenyl)-3-((4-(trifluoromethoxy)phenyl)sulfonamido)propanoate;
(S)—N-(1-(3,4-dichlorophenyl)-2-(dimethylamino)ethyl)-3,5-difluorobenzenesulfonamide;
(S)—N-(1-(2-methoxy-5-methylphenyl)-3-methylbutan-2-yl)-4-(trifluoromethoxy)benzenesulfonamide;
N-(2-(((1S,2S)-2-methylcyclohexyl)oxy)ethyl)-4-(trifluoromethoxy)benzenesulfonamide;
N-(2-(((1S,2R)-2-methylcyclohexyl)oxy)ethyl)-4-(trifluoromethoxy)benzenesulfonamide;
N-(((1S,2S)-2-phenylcyclohexyl)methyl)-4-(trifluoromethoxy)benzenesulfonamide;
N-(((1S,2R)-2-phenylcyclohexyl)methyl)-4-(trifluoromethoxy)benzenesulfonamide;
N-(((1S,3S)-1-(dimethylamino)-3-methylcyclohexyl)methyl)-4-(trifluoromethoxy)benzenesulfonamide;
N-(((1S,3R)-1-(dimethylamino)-3-methylcyclohexyl)methyl)-4-(trifluoromethoxy)benzenesulfonamide;
N-((1S,2S)-2-(methylsulfonyl)-1-(p-tolyl)propyl)-4-(trifluoromethoxy)benzenesulfonamide;
N-((1S,2R)-2-(methylsulfonyl)-1-(p-tolyl)propyl)-4-(trifluoromethoxy)benzenesulfonamide;
N-((1S,2S)-1-(2-chlorophenyl)-1-methoxypropan-2-yl)-4-(trifluoromethoxy)benzenesulfonamide;
N-((1S,2R)-1-(2-chlorophenyl)-1-methoxypropan-2-yl)-4-(trifluoromethoxy)benzenesulfonamide;
ethyl (S)-3-((S)-tetrahydrofuran-3-yl)-2-((4-(trifluoromethoxy)phenyl)sulfonamido)propanoate;
ethyl (S)-3-((R)-tetrahydrofuran-3-yl)-2-((4-(trifluoromethoxy)phenyl)sulfonamido)propanoate;
ethyl (S)-3-((S)-cyclohex-3-en-1-yl)-2-((4-(trifluoromethoxy)phenyl)sulfonamido)propanoate;
ethyl (S)-3-((R)-cyclohex-3-en-1-yl)-2-((4-(trifluoromethoxy)phenyl)sulfonamido)propanoate;
N—((S)-(4-fluorophenyl)((S)-tetrahydrofuran-2-yl)methyl)-4-(trifluoromethoxy)benzenesulfonamide;
N—((S)-(4-fluorophenyl)((R)-tetrahydrofuran-2-yl)methyl)-4-(trifluoromethoxy)benzenesulfonamide;
(S)-4-cyclopropoxy-N-(1-(3,4-dichlorophenyl)-2-(dimethylamino)ethyl)benzenesulfonamide;
(S)—N-(1-(3-chloro-4-fluorophenyl)-2-(dimethylamino)ethyl)-4-(trifluoromethoxy)benzenesulfonamide;
(S)—N-(1-(4-chloro-3-fluorophenyl)-2-(dimethylamino)ethyl)-4-(trifluoromethoxy)benzenesulfonamide;
(S)-4-(N-(1-(3,4-dichlorophenyl)-2-(dimethylamino)ethyl)sulfamoyl)-N-phenylbenzamide;
(S)—N-(1-(3,4-dichlorophenyl)-2-(dimethylamino)ethyl)-4-(phenylamino)benzenesulfonamide;
(S)—N-(1-(4-chlorophenyl)-2-(dimethylamino)ethyl)-4-(4-(trifluoromethyl)phenoxy)benzenesulfonamide;
(S)—N-(2-(dimethylamino)-1-(3-(trifluoromethyl)phenyl)ethyl)-3-nitro-4-(trifluoromethoxy)benzenesulfonamide;
(S)—N-(1-(4-chlorophenyl)-2-(dimethylamino)ethyl)-3-nitro-4-(trifluoromethoxy)benzenesulfonamide;
(S)-4-(cyclopropylmethoxy)-N-(1-(3,4-dichlorophenyl)-2-(dimethylamino)ethyl)benzenesulfonamide;
(S)—N-(1-(3,4-dichlorophenyl)-2-(dimethylamino)ethyl)-4-isopropoxybenzenesulfonamide;
(S)-4-(cyclohexyloxy)-N-(1-(3,4-dichlorophenyl)-2-(dimethylamino)ethyl)benzenesulfonamide;
(S)—N-(1-(3,4-dichlorophenyl)-2-(dimethylamino)ethyl)-4-(pyridin-2-yloxy)benzenesulfonamide;
(S)-4-butyl-N-(1-(3,4-dichlorophenyl)-2-(dimethylamino)ethyl)benzenesulfonamide;
(S)—N-(1-(3,4-dichlorophenyl)-2-(dimethylamino)ethyl)-4-(3,6-dihydro-2H-pyran-4-yl)benzenesulfonamide;
(S)—N-(2-(dimethylamino)-1-(4-(trifluoromethyl)phenyl)ethyl)-4-(trifluoromethoxy)benzenesulfonamide;
(S)—N-(1-(3,4-dichlorophenyl)-2-(dimethylamino)ethyl)-4-(3-fluorophenoxy)benzenesulfonamide;
(S)—N-(1-(3,4-dichlorophenyl)-2-(dimethylamino)ethyl)-4-(4-fluorophenoxy)benzenesulfonamide;
(S)-methyl 5-(N-(1-(3,4-dichlorophenyl)-2-(dimethylamino)ethyl)sulfamoyl)-2-(trifluoromethoxy)benzoate;
(S)—N-(1-(3,4-dichlorophenyl)-2-(4-methylpiperazin-1-yl)ethyl)-4-(4-(trifluoromethyl)phenoxy)benzenesulfonamide;
(S)—N-(1-(4-chlorophenyl)-2-(4-methylpiperazin-1-yl)ethyl)-4-phenoxybenzenesulfonamide;
(S)—N-(1-(4-chlorophenyl)-2-(4-methylpiperazin-1-yl)ethyl)-4-(4-(trifluoromethyl)phenoxy)benzenesulfonamide;
(S)—N-(1-(3,4-dichlorophenyl)-2-(4-methylpiperazin-1-yl)ethyl)-4-phenoxybenzenesulfonamide;
(S)—N-(2-(dimethylamino)-1-(3-(trifluoromethyl)phenyl)ethyl)-4-(4-(trifluoromethyl)phenoxy)benzenesulfonamide;
(S)—N-(1-(3,4-dichlorophenyl)-2-(dimethylamino)ethyl)-6-(trifluoromethoxy)pyridine-3-sulfonamide;
(S)—N-(1-(3,4-dichlorophenyl)-2-(dimethylamino)ethyl)-3-nitro-4-(trifluoromethoxy)benzenesulfonamide;
(S)—N-(1-(3,4-dichlorophenyl)-2-(dimethylamino)ethyl)-4-(pyridin-2-yl)benzenesulfonamide;
(S)—N-(1-(3,4-dichlorophenyl)-2-(dimethylamino)ethyl)-4-nitro-3-(trifluoromethoxy)benzenesulfonamide;
(S)—N-(2-amino-1-(3,4-dichlorophenyl)ethyl)-4-(trifluoromethoxy)benzenesulfonamide hydrochloride;
(S)-tert-butyl (2-(3,4-dichlorophenyl)-2-((4-(trifluoromethoxy)phenyl)sulfonamido)ethyl)carbamate;
(S)—N-((2,2-dimethyl-1-phenylcyclopropyl)methyl)-4-(trifluoromethoxy)benzenesulfonamide;
(S)—N-(1-(3,4-dichlorophenyl)-2-(dimethylamino)ethyl)-2-nitro-3-(trifluoromethoxy)benzenesulfonamide;
4-(trifluoromethoxy)-N-((1S,3S)-3,4,4-trimethylcyclohexyl)benzenesulfonamide;
4-(trifluoromethoxy)-N-((1S,3R)-3,4,4-trimethylcyclohexyl)benzenesulfonamide;
(S)—N-(1-(3,4-dichlorophenyl)-2-(dimethylamino)ethyl)-4-(2-(trifluoromethyl)phenoxy)benzenesulfonamide;
(S)—N-(1-(3,4-dichlorophenyl)-2-(dimethylamino)ethyl)-4-(tetrahydro-2H-pyran-2-yl)benzenesulfonamide;
(S)-3-cyano-N-(1-(3,4-dichlorophenyl)-2-(dimethylamino)ethyl)-4-(trifluoromethoxy)benzenesulfonamide;
(S)—N-(1-(3,4-dichlorophenyl)-2-(4-propoxypiperidin-1-yl)ethyl)-4-(trifluoromethoxy)benzenesulfonamide;
(S)—N-(1-(3,4-dichlorophenyl)-2-(methylamino)ethyl)-4-(trifluoromethoxy)benzenesulfonamide;
(S)—N-(1-(3,4-dichlorophenyl)-3-(dimethylamino)propan-2-yl)-4-(trifluoromethoxy)benzenesulfonamide;
(S)-4-cyclobutoxy-N-(1-(3,4-dichlorophenyl)-2-(dimethylamino)ethyl)benzenesulfonamide;
(S)-3-(3,4-dichlorophenyl)-N,N-dimethyl-2-((4-(trifluoromethoxy)phenyl)sulfonamido)propenamide;
(S)—N-(1-(3,4-dichlorophenyl)-2-(dimethylamino)ethyl)-N-methyl-4-(trifluoromethoxy)benzenesulfonamide;
(S)-4-(cyclopentylamino)-N-(1-(3,4-dichlorophenyl)-2-(dimethylamino)ethyl)benzenesulfonamide;
(S)-4-(cyclobutylamino)-N-(1-(3,4-dichlorophenyl)-2-(dimethylamino)ethyl)benzenesulfonamide;
(S)—N-((3-(3,4-dichlorophenyl)pyrrolidin-3-yl)methyl)-4-(trifluoromethoxy)benzenesulfonamide;
(S)-2-(3,4-dichlorophenyl)-N,N-dimethyl-2-((4-(trifluoromethoxy)phenyl)sulfonamido)acetamide;
(S)—N-(1-(3,4-dichlorophenyl)-2-((4-methoxybenzyl)amino)ethyl)-4-(trifluoromethoxy)benzenesulfonamide;
(S)—N-(1-(4-chlorophenyl)-2-(piperazin-1-yl)ethyl)-4-(trifluoromethoxy)benzenesulfonamide;
(S)—N-(1-(4-chloro-3-fluorophenyl)-2-(piperazin-1-yl)ethyl)-4-(trifluoromethoxy)benzenesulfonamide;
(S)—N-(1-(4-chlorophenyl)-2-(piperazin-1-yl)ethyl)-6-(trifluoromethoxy)pyridine-3-sulfonamide;
N—((S)-2-((1R,4R)-2,5-diazabicyclo[2.2.1]heptan-2-yl)-1-(3,4-dichlorophenyl)ethyl)-4-(trifluoromethoxy)benzenesulfonamide;
N—((S)-2-((1S,4S)-2,5-diazabicyclo[2.2.1]heptan-2-yl)-1-(3,4-dichlorophenyl)ethyl)-4-(trifluoromethoxy)benzenesulfonamide;
(S)—N-((2-phenylpyrrolidin-2-yl)methyl)-4-(trifluoromethoxy)benzenesulfonamide;
(S)—N-(1-(4-fluorophenyl)-2-(piperazin-1-yl)ethyl)-4-(trifluoromethoxy)benzenesulfonamide;
(S)—N-(2-(4-fluorophenyl)-2-(piperazin-1-yl)ethyl)-4-(trifluoromethoxy)benzenesulfonamide;
(S)—N-(1-(3,4-dichlorophenyl)-2-(dimethylamino)ethyl)-3-nitro-4-(trifluoromethyl)benzenesulfonamide;
(S)—N-(1-(3,4-dichlorophenyl)-2-(dimethylamino)ethyl)-4-propoxybenzenesulfonamide;
(S)-4-(cyclohexylamino)-N-(1-(3,4-dichlorophenyl)-2-(dimethylamino)ethyl)benzenesulfonamide; and
(S)—N-(1-(3,4-dichlorophenyl)-2-(4-methylpiperazin-1-yl)ethyl)-4-(4-fluorophenoxy)benzenesulfonamide;
or a salt, solvate, isotopologue, or tautomer thereof.

Embodiment 30 provides a compound selected from the group consisting of
N-(3,3,3-trifluoro-1-phenylpropyl)-4-(trifluoromethoxy)benzenesulfonamide;
(R)—N-(3,3,3-trifluoro-1-phenylpropyl)-4-(trifluoromethoxy)benzenesulfonamide;
(S)—N-(3,3,3-trifluoro-1-phenylpropyl)-4-(trifluoromethoxy)benzenesulfonamide;
N-(3-hydroxy-1-phenylpropyl)-4-(trifluoromethoxy)benzenesulfonamide;
(R)—N-(3-hydroxy-1-phenylpropyl)-4-(trifluoromethoxy)benzenesulfonamide;
(S)—N-(3-hydroxy-1-phenylpropyl)-4-(trifluoromethoxy)benzenesulfonamide;
N-(2-cyclopropyl-1-phenylethyl)-4-(trifluoromethoxy)benzenesulfonamide;
(R)—N-(2-cyclopropyl-1-phenylethyl)-4-(trifluoromethoxy)benzenesulfonamide;
(S)—N-(2-cyclopropyl-1-phenylethyl)-4-(trifluoromethoxy)benzenesulfonamide;
methyl 2-methyl-3-phenyl-3-((4-(trifluoromethoxy)phenyl)sulfonamido)propanoate;
methyl (2R,3R)-2-methyl-3-phenyl-3-((4-(trifluoromethoxy)phenyl)sulfonamido)propanoate;
methyl (2R,3S)-2-methyl-3-phenyl-3-((4-(trifluoromethoxy)phenyl)sulfonamido)propanoate;
methyl (2S,3S)-2-methyl-3-phenyl-3-((4-(trifluoromethoxy)phenyl)sulfonamido)propanoate;
methyl (2S,3R)-2-methyl-3-phenyl-3-((4-(trifluoromethoxy)phenyl)sulfonamido)propanoate;
ethyl 3-(p-tolyl)-3-((4-(trifluoromethoxy)phenyl)sulfonamido)propanoate;
ethyl (R)-3-(p-tolyl)-3-((4-(trifluoromethoxy)phenyl)sulfonamido)propanoate;
ethyl (S)-3-(p-tolyl)-3-((4-(trifluoromethoxy)phenyl)sulfonamido)propanoate;
N-(4,4,4-trifluoro-3-hydroxy-3-(trifluoromethyl)butyl)-4-(trifluoromethoxy)benzenesulfonamide;
N-((1-(2-methoxyethyl)cyclohexyl)methyl)-4-(trifluoromethoxy)benzenesulfonamide;
N-(2-(diisobutylamino)ethyl)-4-(trifluoromethoxy)benzenesulfonamide;
N-(3-(benzyl(butyl)amino)propyl)-4-(trifluoromethoxy)benzenesulfonamide;
N-(1-(2,6-dichlorophenyl)propan-2-yl)-4-(trifluoromethoxy)benzenesulfonamide;
(R)—N-(1-(2,6-dichlorophenyl)propan-2-yl)-4-(trifluoromethoxy)benzenesulfonamide;
(S)—N-(1-(2,6-dichlorophenyl)propan-2-yl)-4-(trifluoromethoxy)benzenesulfonamide;
tert-butyl 2,2-dimethyl-6-(((4-(trifluoromethoxy)phenyl)sulfonamido)methyl)morpholine-4-carboxylate;
(R)-tert-butyl 2,2-dimethyl-6-(((4-(trifluoromethoxy)phenyl)sulfonamido)methyl)morpholine-4-carboxylate;
(S)-tert-butyl 2,2-dimethyl-6-(((4-(trifluoromethoxy)phenyl)sulfonamido)methyl)morpholine-4-carboxylate;
N-(3-((furan-2-ylmethyl)((tetrahydrofuran-2-yl)methyl)amino)propyl)-4-(trifluoromethoxy)benzenesulfonamide;
(R)—N-(3-((furan-2-ylmethyl)((tetrahydrofuran-2-yl)methyl)amino)propyl)-4-(trifluoromethoxy)benzenesulfonamide;
(S)—N-(3-((furan-2-ylmethyl)((tetrahydrofuran-2-yl)methyl)amino)propyl)-4-(trifluoromethoxy)benzenesulfonamide;
N-((1-(2-(benzyloxy)ethyl)cyclopropyl)methyl)-4-(trifluoromethoxy)benzenesulfonamide;
N-(3-(2-(benzyloxy)ethoxy)propyl)-4-(trifluoromethoxy)benzenesulfonamide;
N-(1-(1-methylpiperidin-2-yl)propan-2-yl)-4-(trifluoromethoxy)benzenesulfonamide;
N-(2-((2-methylcyclohexyl)oxy)ethyl)-4-(trifluoromethoxy)benzenesulfonamide;
N-((1-((dimethylamino)methyl)cyclopropyl)methyl)-4-(trifluoromethoxy)benzenesulfonamide;
N-((1-(dimethylamino)-3-methylcyclohexyl)methyl)-4-(trifluoromethoxy)benzenesulfonamide;
N-(2-(2,2-dioxidobenzo[c][1,2,5]thiadiazol-1(3H)-yl)ethyl)-4-(trifluoromethoxy)benzenesulfonamide;
N-((3-methylcyclohexyl)(o-tolyl)methyl)-4-(trifluoromethoxy)benzenesulfonamide;
(R)—N-((3-methylcyclohexyl)(o-tolyl)methyl)-4-(trifluoromethoxy)benzenesulfonamide;
(S)—N-((3-methylcyclohexyl)(o-tolyl)methyl)-4-(trifluoromethoxy)benzenesulfonamide;
N-((2-methylcyclopropyl)(phenyl)methyl)-4-(trifluoromethoxy)benzenesulfonamide;
(R)—N-((2-methylcyclopropyl)(phenyl)methyl)-4-(trifluoromethoxy)benzenesulfonamide;
(S)—N-((2-methylcyclopropyl)(phenyl)methyl)-4-(trifluoromethoxy)benzenesulfonamide;
N-(2-(methylsulfonyl)-1-(p-tolyl)propyl)-4-(trifluoromethoxy)benzenesulfonamide;
tert-butyl (1-phenyl-2-((4-(trifluoromethoxy)phenyl)sulfonamido)propyl)carbamate;
tert-butyl ((1R,2R)-1-phenyl-2-((4-(trifluoromethoxy)phenyl)sulfonamido)propyl)carbamate;
tert-butyl ((1R,2S)-1-phenyl-2-((4-(trifluoromethoxy)phenyl)sulfonamido)propyl)carbamate;
tert-butyl ((1S,2S)-1-phenyl-2-((4-(trifluoromethoxy)phenyl)sulfonamido)propyl)carbamate;
tert-butyl ((1S,2R)-1-phenyl-2-((4-(trifluoromethoxy)phenyl)sulfonamido)propyl)carbamate;
tert-butyl (2-phenyl-2-((4-(trifluoromethoxy)phenyl)sulfonamido)ethyl)carbamate;
tert-butyl (R)-(2-phenyl-2-((4-(trifluoromethoxy)phenyl)sulfonamido)ethyl)carbamate;
tert-butyl (S)-(2-phenyl-2-((4-(trifluoromethoxy)phenyl)sulfonamido)ethyl)carbamate;
N-(2-amino-1-phenylethyl)-4-(trifluoromethoxy)benzenesulfonamide;
(R)—N-(2-amino-1-phenylethyl)-4-(trifluoromethoxy)benzenesulfonamide;
(S)—N-(2-amino-1-phenylethyl)-4-(trifluoromethoxy)benzenesulfonamide;
N-(1-phenyl-2-(4-propoxypiperidin-1-yl)ethyl)-4-(trifluoromethoxy)benzenesulfonamide;
(R)—N-(1-phenyl-2-(4-propoxypiperidin-1-yl)ethyl)-4-(trifluoromethoxy)benzenesulfonamide;
(S)—N-(1-phenyl-2-(4-propoxypiperidin-1-yl)ethyl)-4-(trifluoromethoxy)benzenesulfonamide;
1-methyl-N-(2-phenyl-2-((4-(trifluoromethoxy)phenyl)sulfonamido)ethyl)-1H-pyrrole-3-sulfonamide;
(R)-1-methyl-N-(2-phenyl-2-((4-(trifluoromethoxy)phenyl)sulfonamido)ethyl)-1H-pyrrole-3-sulfonamide;
(S)-1-methyl-N-(2-phenyl-2-((4-(trifluoromethoxy)phenyl)sulfonamido)ethyl)-1H-pyrrole-3-sulfonamide;
N-(1-(dimethylamino)propan-2-yl)-3-nitro-4-(trifluoromethoxy)benzenesulfonamide;
(R)—N-(1-(dimethylamino)propan-2-yl)-3-nitro-4-(trifluoromethoxy)benzenesulfonamide; and
(S)—N-(1-(dimethylamino)propan-2-yl)-3-nitro-4-(trifluoromethoxy)benzenesulfonamide;
or a salt, solvate, isotopologue, or tautomer thereof.

Embodiment 31 provides a pharmaceutical composition comprising at least one compound of any one of Embodiments 1-30 and at least one pharmaceutically acceptable carrier.

Embodiment 32 provides a method of treating, preventing, and/or ameliorating a PP2A-related disease in a subject, the method comprising administering to a subject in need thereof a therapeutically effective amount of the compound of any one of Embodiments 1-30 or the pharmaceutical composition of Embodiment 31.

Embodiment 33 provides the method of Embodiment 32, wherein the PP2A-related disease is selected from the group consisting of cancer, diabetes, autoimmune disease, solid organ transplant rejection, graft vs host disease, chronic obstructive pulmonary disease (COPD), non-alcoholic fatty liver disease, abdominal aortic aneurysm, chronic liver disease, heart failure, neurodegenerative disease, and cardiac hypertrophy.

Embodiment 34 provides the method of Embodiment 32 or 33, wherein the subject is a mammal.

Embodiment 35 provides the method of Embodiment 34, wherein the mammal is a human.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A compound of formula (I), or a salt, solvate, enantiomer, diastereoisomer, isotopologue, or tautomer thereof: 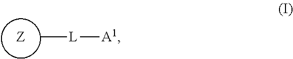
wherein:
A¹ is selected from the group consisting of 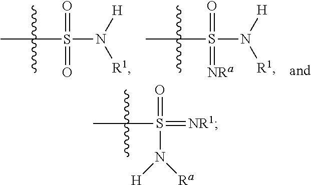
L is a linker comprising a bond, C₁-C₆ alkylene, or C₂-C₆ alkenylene;
ring Z is optionally substituted phenyl, optionally substituted naphthyl, optionally substituted pyridyl, or optionally substituted cyclohexyl,
wherein each optional substituent is at least one substituent selected from the group consisting of C₁-C₆ alkyl, C₁-C₆ haloalkyl, C₁-C₆ aminoalkyl, C₁-C₆ hydroxyalkyl, C₂-C₆ alkenyl, C₂-C₆ alkynyl, C₁-C₆ alkoxy, C₁-C₆ haloalkoxy, C₆-C₁₀ aryl, C₄-C₁₀ heteroaryl, C₆-C₁₀ aryloxy, C₄-C₁₀ heteroaryloxy F, Cl, Br, I, OH, CN, N(R^{a})(R^{b}), NO₂, C(═O)OR^{a}, and C(═O)N(R^{a})(R^{b}),
wherein each optional C₆-C₁₀ aryl, C₄-C₁₀ heteroaryl, C₄-C₁₀ heteroaryloxy, or C₆-C₁₀ aryloxy substituent is independently optionally substituted with at least one substituent selected from the group consisting of C₁-C₃ alkyl, C₁-C₃ haloalkyl, C₁-C₃ alkoxy, C₁-C₃ haloalkoxy, F, Cl, Br, I, OH, CN, N(R^{a})(R^{b}), NO₂, OH, C(═O)OR^{a}, and C(═O)N(R^{a})(R^{b}),
wherein two adjacent substituents of ring Z may optionally combine to provide a 5 to 8-membered fused ring;
R¹ is 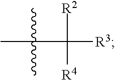
R² is selected from the group consisting of optionally substituted C₁-C₆ alkyl, optionally substituted C₁-C₆ haloalkyl, optionally substituted C₁-C₆ aminoalkyl, optionally substituted C₁-C₆ hydroxyalkyl, optionally substituted C₂-C₆ alkenyl, optionally substituted C₅-C₈ cycloalkenyl, optionally substituted C₂-C₆ alkynyl, optionally substituted C₁-C₆ alkoxy, optionally substituted C₁-C₆ haloalkoxy, optionally substituted C₃-C₁₂ cycloalkyl, optionally substituted C₂-C₁₂ heterocycloalkyl, optionally substituted C₆-C₁₀ aryl, and optionally substituted C₄-C₁₀ heteroaryl,
wherein each optional substituent in R² is independently selected from the group consisting of C₁-C₃ alkyl, C₁-C₃ haloalkyl, C₁-C₃ alkoxy, C₃-C₅ cycloalkoxy, C₃-C₁₂ heterocycloalkyl, C₁-C₃ haloalkoxy, F, Cl, Br, OR^{b}, CN, NO₂, C(═O)OR^{a}, C(═O)N(R^{a})₂, S(═O)₂R^{b}, NR^{a}S(═O)₂R^{b}, C₄-C₁₀ heteroaryl, benzyl, and phenyl, wherein the benzyl and/or phenyl are each independently optionally substituted with at least one substituent selected from the group consisting of C₁-C₆ alkyl, C₁-C₆ alkoxy, C₁-C₃ haloalkyl, C₁-C₃ haloalkoxy, and halogen;
R³ is selected from the group consisting of H, C(═O)R^{a}, C(═O)OR^{a}, C(═O)N(R^{a})₂, NR^{a}C(═)OR, optionally substituted C₁-C₆ alkyl, optionally substituted C₁-C₆ alkoxy, optionally substituted C₁-C₆ haloalkyl, optionally substituted C₁-C₆ aminoalkyl, optionally substituted C₁-C₆ hydroxyalkyl, optionally substituted C₃-C₁₂ cycloalkyl, and optionally substituted C₂-C₁₂ heterocycloalkyl,
wherein each optional substituent in R³ is selected from the group consisting of C₁-C₃ alkyl, C₂-C₁₂ heterocycloalkyl, C₁-C₃ haloalkyl, C₁-C₃ alkoxy, C₁-C₃ haloalkoxy, benzyl, C₆-C₁₀ aryl, C₄-C₁₀ heteroaryl, F, Cl, Br, OH, CN, NO₂, C(═O)OR^{a}, C(═O)N(R^{a})(R^{b}), NR^{a}C(═O)OR^{a}, S(═O)₂R^{b}, and NR^{a}S(═O)₂R^{b},
R⁴ is H or C₁-C₆ alkyl;
each occurrence of R^{a} is independently selected from the group consisting of H, C₁-C₆ alkyl, C₃-C₆ cycloalkyl, benzyl, and C₆-C₁₀ aryl;
each occurrence of R^{b} is independently selected from the group consisting of H, optionally substituted C₁-C₆ alkyl, optionally substituted C₂-C₁₂ heterocycloalkyl, optionally substituted benzyl, optionally substituted C₂-C₅ heteroaryl, optionally substituted phenyl, and optionally substituted naphthyl,
wherein each optional substituent in R^{b} is independently selected from the group consisting of C₁-C₃ alkyl, C₁-C₃ haloalkyl, C₁-C₃ alkoxy, C₁-C₃ haloalkoxy, F, Cl, Br, I, OH, CN, NO₂, C(═O)OR^{a}, and C(═O)N(R^{a})₂;
wherein no more than one of R² and R³ is CH₂OH;
wherein if R³ is H and R⁴ is H, R³ is CH₃ and R⁴ is H, or R³ is H and R⁴ is CH₃, then one of the following applies:
(a) R² comprises at least one substituent selected from the group consisting of C₆-C₁₀ aryl and C₄-C₁₀ heteroaryl, wherein the aryl and/or heteroaryl are each independently substituted with at least one substituent selected from the group consisting of OR^{b}, N(R^{a})(R^{a}), C₁-C₆ alkyl, C₁-C₃ haloalkyl, C₁-C₃ haloalkoxy, C₂-C₁₂ heterocycloalkyl, F, Cl, Br, and S(═O)₂R^{b}; or
(b) R² comprises at least one substituent selected from the group consisting of C₃-C₆ cycloalkyl and C₂-C₁₂ heterocycloalkyl, and at least one substituent selected from the group consisting of C₆-C₁₀ aryl and C₄-C₁₀ heteroaryl;
wherein if L is a bond, R³ is H, R⁴ is H, and A¹ is 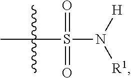
then the optionally substituted C₂-C₁₂ heterocycloalkyl in R² comprises no more than one phenyl substituted with at least one halogen;
wherein if Z is optionally substituted phenyl, L is a bond, A¹ is 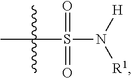
and R² is optionally substituted C₆-C₁₀ aryl, then one of the following applies:
(a) the phenyl in Z is substituted with at least one substituent selected from the group consisting of C₁-C₆ haloalkyl, C₁-C₆ aminoalkyl, C₁-C₆ hydroxyalkyl, C₂-C₆ alkenyl, C₂-C₆ alkynyl, C₁-C₆ alkoxy, C₁-C₆ haloalkoxy, C₆-C₁₀ aryl, C₃-C₆ heterocycloalkyl, C₄-C₁₀ heteroaryl, C₆-C₁₀ aryloxy, C₄-C₁₀ heteroaryloxy F, Cl, Br, I, OH, CN, N(R^{a})(R^{b}), NO₂, C(═O)OR^{a}, and C(═O)N(R^{a})(R^{b}), and
the C₆-C₁₀ aryl in R² is substituted with at least one substituent selected from the group consisting of C₁-C₃ haloalkyl, C₁-C₃ alkoxy, C₃-C₅ cycloalkoxy, C₃-C₁₂ heterocycloalkyl, C₁-C₃ haloalkoxy, F, Cl, Br, OR^{b}, CN, NO₂, C(═O)OR^{a}, C(═O)N(R^{a})₂, S(═O)₂R^{b}, NR^{a}S(═O)₂R^{b}, C₄-C₁₀ heteroaryl, and benzyl;
(b) the phenyl in Z is substituted with at least one substituent, and the C₆-C₁₀ aryl in R² is substituted with at least two substituents; or
(c) the phenyl in Z is substituted with at least one trifluoromethoxy substituent, the C₆-C₁₀ aryl in R² is substituted with at least one substituent, and R³ is optionally substituted C₁-C₆ aminoalkyl;
wherein if Z is 4-trifluoromethoxyphenyl, L is a bond, and A¹ is 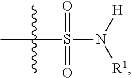
then each of the following apply:
(a) if R³ and R⁴ are each independently H, then R² is not selected from the group consisting of phenyl, 2-difluoromethoxyphenyl, 2-methylphenyl, 2-cyanophenyl, 2-methoxyphenyl, 2-trifluoromethoxyphenyl, 2-chlorophenyl, 2-fluorophenyl, 2-trifluoromethylphenyl, 3-methylphenyl, 3-methoxyphenyl, 3-bromophenyl, 3-chlorophenyl, 3-trifluoromethoxyphenyl, 3-trifluoromethylphenyl, 4-isobutyramido-phenyl, 4-tert-butylphenyl, 4-methoxyphenyl, 4-chlorophenyl, 4-bromophenyl, 4-carboxyphenyl, 4-trifluoromethylphenyl, 4-methylphenyl, 4-fluorophenyl, 2-fluoro-3-trifluoromethylphenyl, 2,3-dimethylphenyl, 2,3-dichlorophenyl, 2-methyl-4-chlorophenyl, 2-chloro-4-fluorophenyl, 2,4-dimethoxyphenyl, 2-methoxy-4-(2-methoxy-5-fluorophenyl)phenyl, 2-methoxy-4-chlorophenyl, 2-fluoro-4-trifluoromethylphenyl, 2,6-difluorophenyl, 2-chloro-6-methylphenyl, 2,6-dimethoxyphenyl, 3,4-ethylenedioxyphenyl, 3-fluoro-4-trifluoromethylphenyl, 3,4-difluorophenyl, 3-fluoro-4-methylphenyl, 3-methoxy-4-ethoxyphenyl, 3,4-dimethoxyphenyl, 3-chloro-4-methylphenyl, 3-methyl-4-fluorophenyl, 3-chloro-4-fluorophenyl, 3,5-difluorophenyl, 3,5-dichlorophenyl, 3,5-dimethoxyphenyl, 3,5-dimethylphenyl, 3,6-dichlorophenyl, 3,6-difluorophenyl, 3,6-dimethoxyphenyl, 3-chloro-6-methylphenyl, 2,3,4-trifluorophenyl, 3,4,5-trifluorophenyl, 2,6-difluoro-3-chlorophenyl, 1-((5-ethanolyl)thiophen-2-yl)cyclopentyl, and 1-(thiophen-2-yl)cyclopentyl;
(b) if R³ is H and R⁴ is methyl or R³ is methyl and R⁴ is H, then R² is not selected from the group consisting of phenyl, naphthyl, 3-methoxyphenyl, 2,4-difluorophenyl, 3,4-methylenedioxyphenyl, 3,4-(1,2-ethylenedioxy)phenyl, 3-fluoro-4-methoxyphenyl, 3,6-dimethoxyphenyl, 2-methylphenyl, 2-methoxyphenyl, 4-methylphenyl, 4-bromophenyl, 4-fluorophenyl, 4-methoxyphenyl, 4-ethoxyphenyl, 4-(methanesulfonyl)phenyl, 4-(3-imidazolyl)phenyl, and 4-(2-(1,2,4-triazol)-yl)phenyl;
(c) if R³ is H and R⁴ is ethyl or R³ is selected from the group consisting of ethyl and cyclopropyl and R⁴ is H, then R² is not selected from the group consisting of phenyl and 4-methoxyphenyl;
(d) if R³ is selected from the group consisting of hydroxymethyl, cyano, 1-(1,2,4-triazol)-yl, and 1-(1,2,5-triazol)-yl, and R⁴ is H, then R² is not phenyl;
(e) if R³ is CH₂C(═O)OH and R⁴ is H, then R² is not selected from the group consisting of phenyl, 2-chlorophenyl, 4-methylphenyl, 4-fluorophenyl, 4-chlorophenyl, 4-tert-butylphenyl, 4-methoxyphenyl, and 3,4-dimethoxyphenyl;
(f) if R³ is CH₂C(═O)OCH₃ and R⁴ is H, then R² is not selected from the group consisting of cyclohexyl, phenyl, 4-chlorophenyl, 4-tert-butylphenyl, 4-methylphenyl, 4-bromophenyl, 4-fluorophenyl, and 3-bromophenyl;
(g) if R³ is CH₂C(═O)OCH₂CH₃ and R⁴ is H, then R² is not selected from the group consisting of phenyl, 2-chlorophenyl, 4-fluorophenyl, and 4-bromophenyl;
(h) if R³ is CH₂(C═O)OCH₂Ph and R⁴ is H, then R² is not 2-chlorophenyl;
(i) if R³ is CH₂(C═O)OCH(CH₃)₂ and R⁴ is H, then R² is not 2-nitrophenyl; and
(j) if R³ is selected from the group consisting of C(═O)NH₂, C(═O)OCH₃, and C(═O)OCH₂CH₃ and R⁴ is H, then R² is not phenyl; and
wherein if Z is 4-difluoromethoxyphenyl, L is a bond, and A¹ is 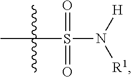
then each of the following apply:
(a) if R³ is CH₂(C═O)OH and R⁴ is H, then R² is not 2-chlorophenyl;
(b) if R³ is CH₂(C═O)OCH₃ and R⁴ is H, then R² is not selected from the group consisting of cyclohexyl, 4-chlorophenyl, and 4-bromophenyl;
(c) if R³ is CH₂(C═O)OCH₂CH₃ and R⁴ is H, then R² is not selected from the group consisting of phenyl, 2-chlorophenyl, and 4-bromophenyl; and
(d) if R³ is CH₂(C═O)OCH₂Ph and R⁴ is H, then R² is not 2-chlorophenyl.

2. The compound of claim 1, wherein L is selected from the group consisting of —CH₂—, —CH₂CH₂—, and —CH═CH—.

3. The compound of claim 1, wherein A¹ is selected from the group consisting of

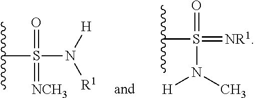

4. The compound of claim 1, wherein R¹ is selected from the group consisting of 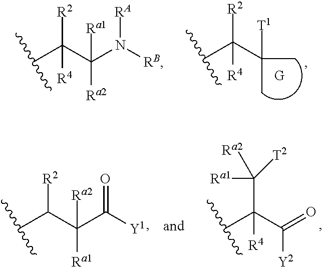
wherein:
R², if present, is selected from the group consisting of optionally substituted C₆-C₁₀ aryl and optionally substituted C₄-C₁₀ heteroaryl,
wherein each optional substituent in R² is independently selected from the group consisting of C₁-C₃ alkyl, C₁-C₃ haloalkyl, C₁-C₃ alkoxy, C₃-C₅ cycloalkoxy, C₁-C₃ haloalkoxy, F, Cl, Br, OH, CN, NO₂, C(═O)OR^{a}, C(═O)N(R^{a})₂, S(═O)₂R^{b}, NR^{a}S(═O)₂R^{b}, C₄-C₁₀ heteroaryl, benzyl, and phenyl, wherein the benzyl and/or phenyl are each independently optionally substituted with at least one substituent selected from the group consisting of C₁-C₆ alkyl, C₁-C₆ alkoxy, C₁-C₃ haloalkyl, C₁-C₃ haloalkoxy, and halogen;
R³, if present, is selected from the group consisting of H and C₁-C₃ alkyl;
R⁴, if present, is selected from the group consisting of H and C₁-C₃ alkyl;
R^{A} and R^{B}, if present, are each independently selected from the group consisting of H, optionally substituted C₁-C₆ alkyl, optionally substituted benzyl, and C(═O)OR^{a},
wherein each optional substituent in each of R^{A} and R^{B} is independently selected from the group consisting of C₁-C₆ alkyl and C₁-C₆ alkoxy, and
wherein R^{A} and R^{B} may combine with the N to which they are bound to form a C₂-C₈ heterocycloalkyl;
R^{a1} and R^{a2}, if present, are each independently selected from the group consisting of H and C₁-C₃ alkyl;
T¹ and T², if present, are each independently phenyl optionally substituted with at least one selected from the group consisting of halogen, C₁-C₃ alkyl, C₁-C₃ haloalkyl, and C₁-C₃ haloalkoxy;
Ring G, if present, is selected from the group consisting of C₃-C₆ cycloalkyl and C₂-C₆ heterocycloalkyl, wherein ring G is optionally substituted with C₁-C₃ alkyl; and
Y¹ and Y², if present, are each independently selected from the group consisting of OR^{a} and N(R^{a})(R^{a}).

5. The compound of claim 4, wherein R² is selected from the group consisting of:

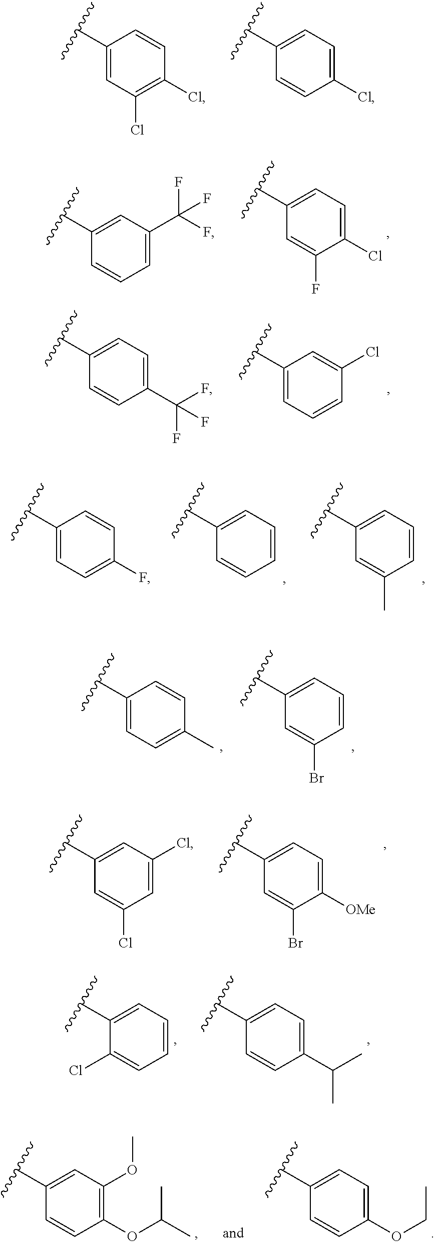

6. The compound of claim 4, wherein R³ is selected from the group consisting of H and CH₃.

7. The compound of claim 4, wherein R⁴ is selected from the group consisting of H and CH₃.

8. The compound of claim 4, wherein at least one of R^{a1} and R^{a2} is H.

9. The compound of claim 4, wherein T¹ is selected from the group consisting of

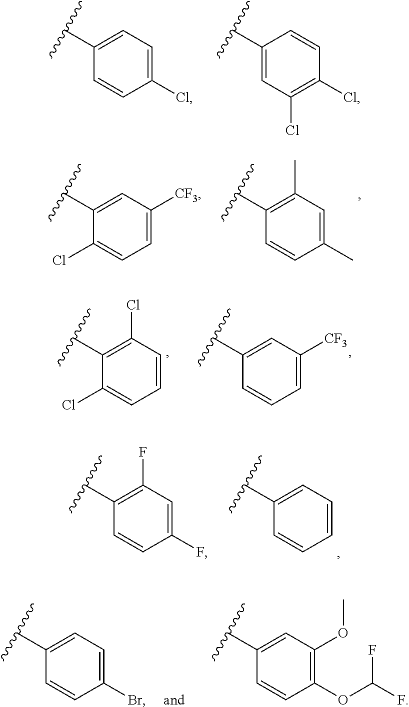

10. The compound of claim 4, wherein T² is selected from the group consisting of

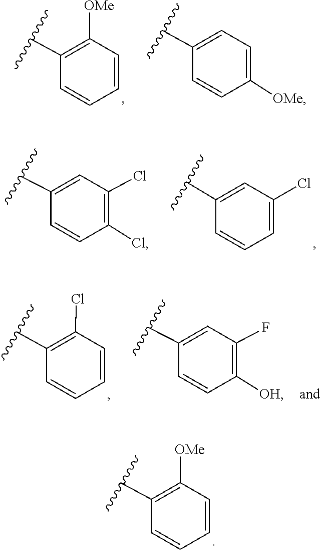

11. The compound of claim 4, wherein Y¹ is selected from the group consisting of OH, OCH₃, and OCH₂CH₃.

12. The compound of claim 4, wherein Y² is selected from the group consisting of OCH₃ and OCH₂CH₃.

13. The compound of claim 4, wherein ring G is selected from the group consisting of

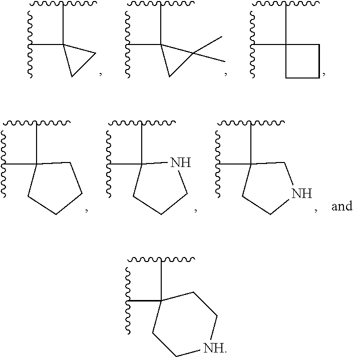

14. The compound of claim 4, wherein one of the following applies:
(a) R^{A} and R^{B} are each independently selected from the group consisting of H, CH₃, C(═O)O(C(CH₃)₃), 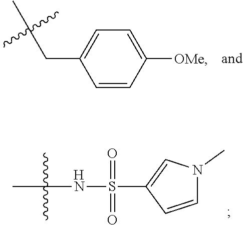
or
(b) R^{A} and R^{B} combine with the N atom to which they are bound to form one selected from the group consisting of

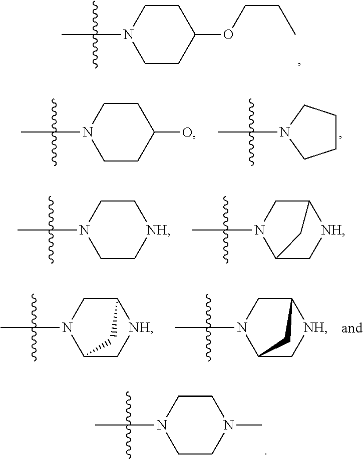

15. The compound of claim 1, wherein the compound of formula (I) is a compound of formula (II): 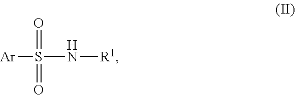
wherein:
Ar is phenyl, naphthyl, or pyridyl, any one of which is optionally substituted with at least one substituent selected from the group consisting of C₁-C₆ alkyl, C₁-C₆ haloalkyl, C₁-C₆ aminoalkyl, C₁-C₆ hydroxyalkyl, C₂-C₆ alkenyl, C₂-C₆ alkynyl, C₁-C₆ alkoxy, C₁-C₆ haloalkoxy, C₆-C₁₀ aryl, C₆-C₁₀ aryloxy, F, Cl, Br, I, OH, NH₂, CN, NO₂, C(═O)OR^{a}, and C(═O)N(R^{a})₂,
wherein each C₆-C₁₀ aryl, C₄-C₁₀ heteroaryl, or C₆-C₁₀ aryloxy substituent is independently optionally substituted with at least one substituent selected from the group consisting of C₁-C₃ alkyl, C₁-C₃ haloalkyl, C₁-C₃ alkoxy, C₁-C₃ haloalkoxy, F, Cl, Br, I, OH, CN, NO₂, C(═O)OR^{a}, and C(═O)N(R^{a})(R^{a}), and
wherein two adjacent substituents of ring Z may optionally combine to provide a 4 to 8-membered fused ring.

16. The compound of claim 15, wherein Ar is selected from the group consisting of: 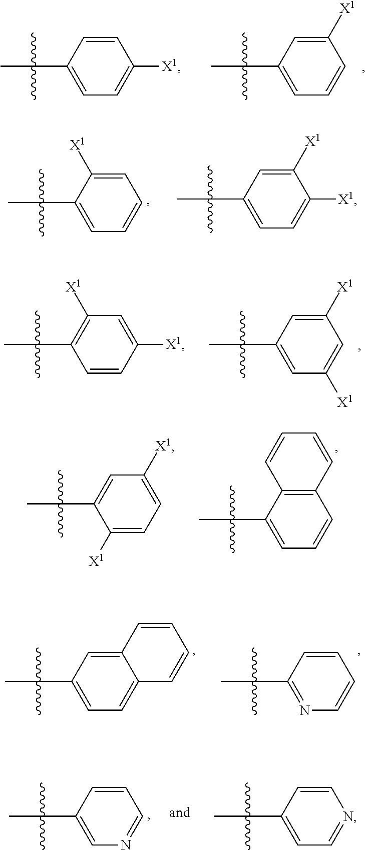
wherein each occurrence of X¹ is independently selected from the group consisting of methyl, ethyl, i-propyl, n-propyl, n-butyl, n-pentyl, Cl, F, OCH₂CH₃, OCH₂CH₂CH₃, OCH(CH₃)₂, CF₃, OCF₃, OCHF₂, CN, NO₂, C(═O)OR^{a}, C(═O)NHR^{a}, NH₂,

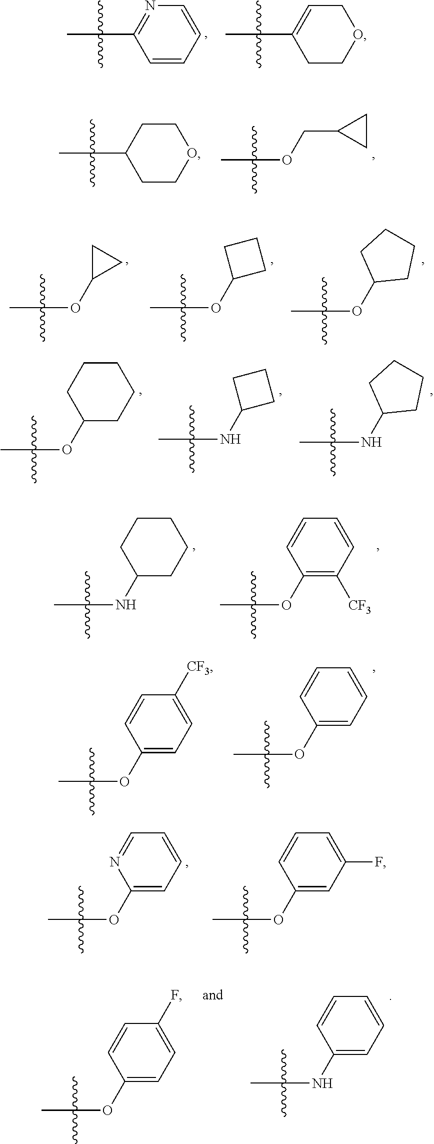

17. The compound of claim 15, wherein Ar is selected from the group consisting of:

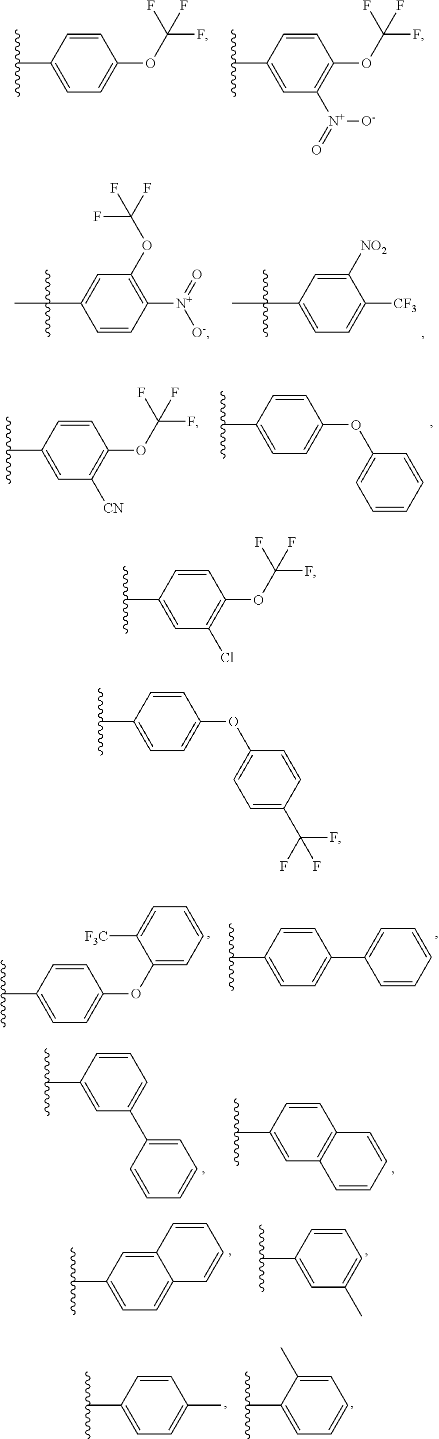

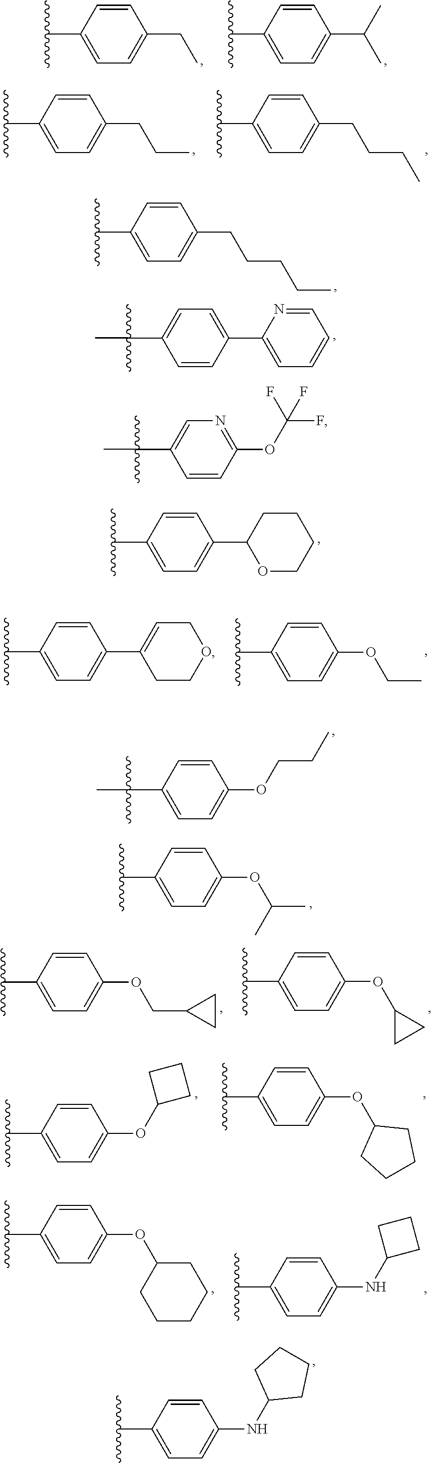

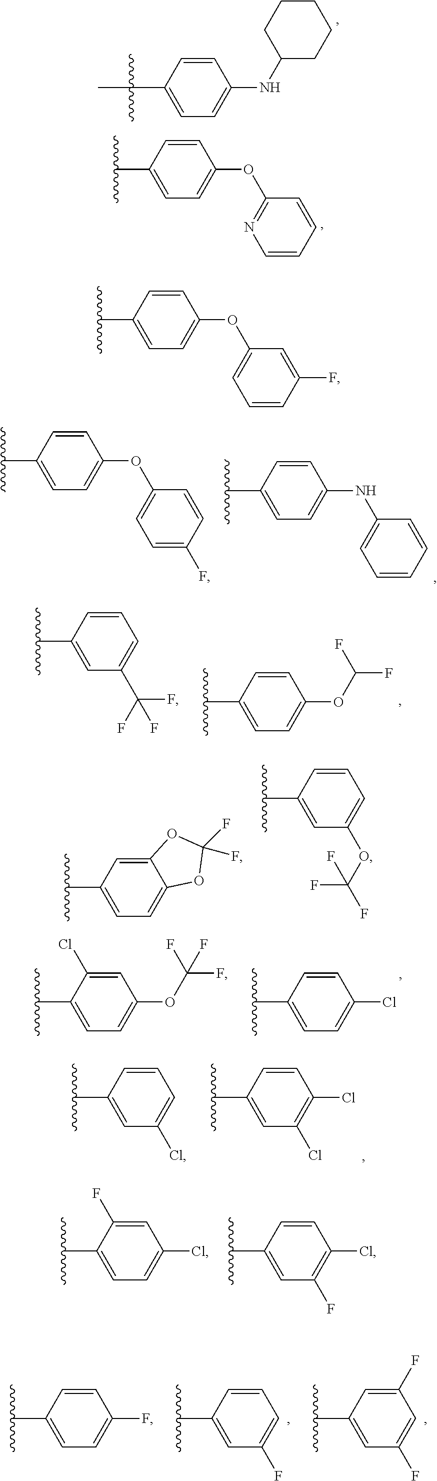

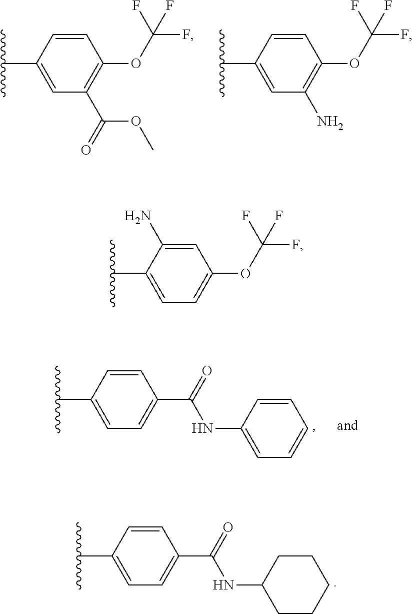

18. The compound of claim 15, wherein R² is selected from the group consisting of:

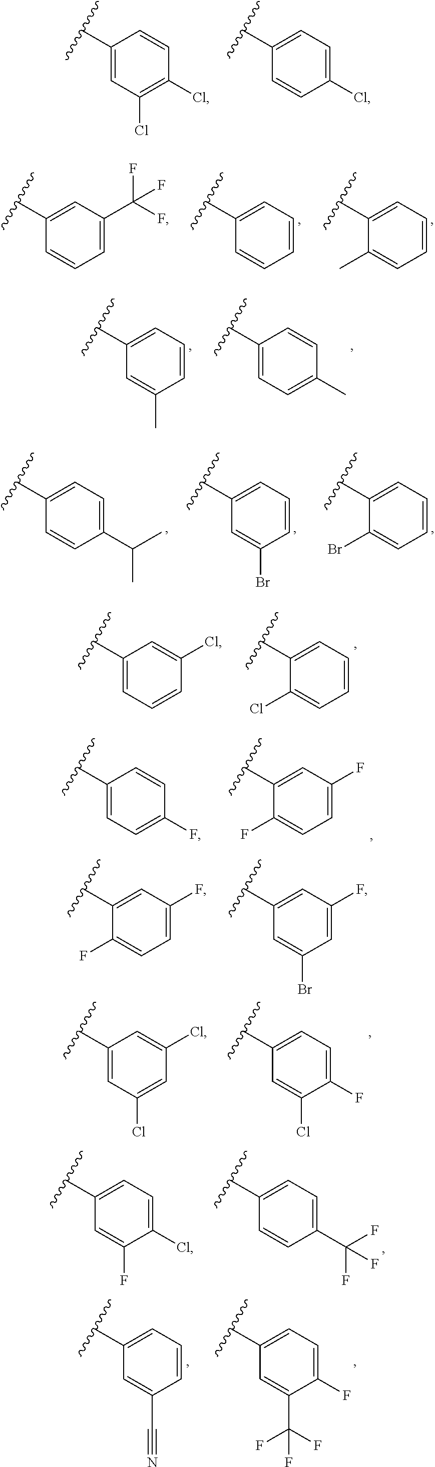

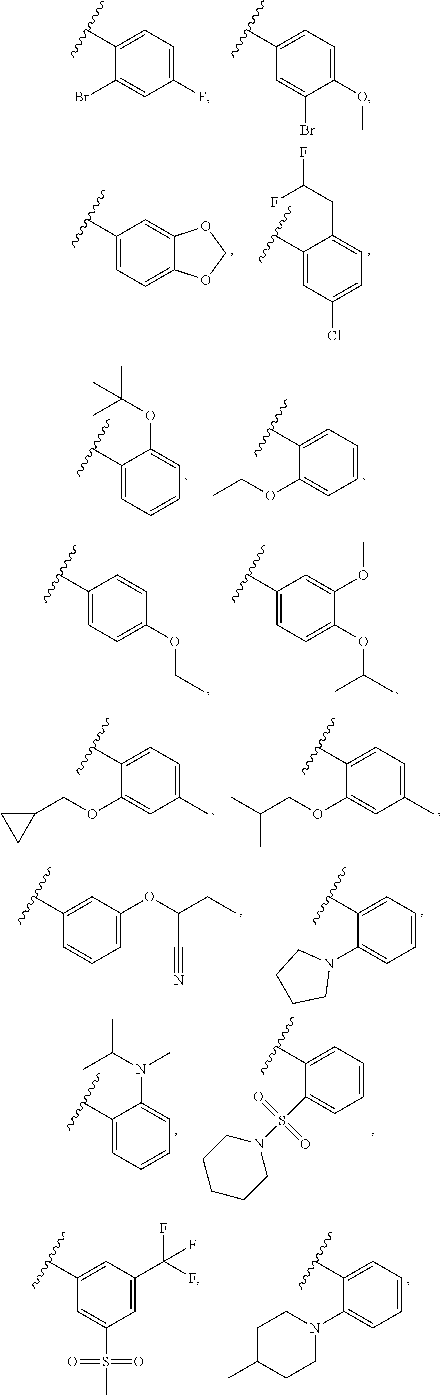

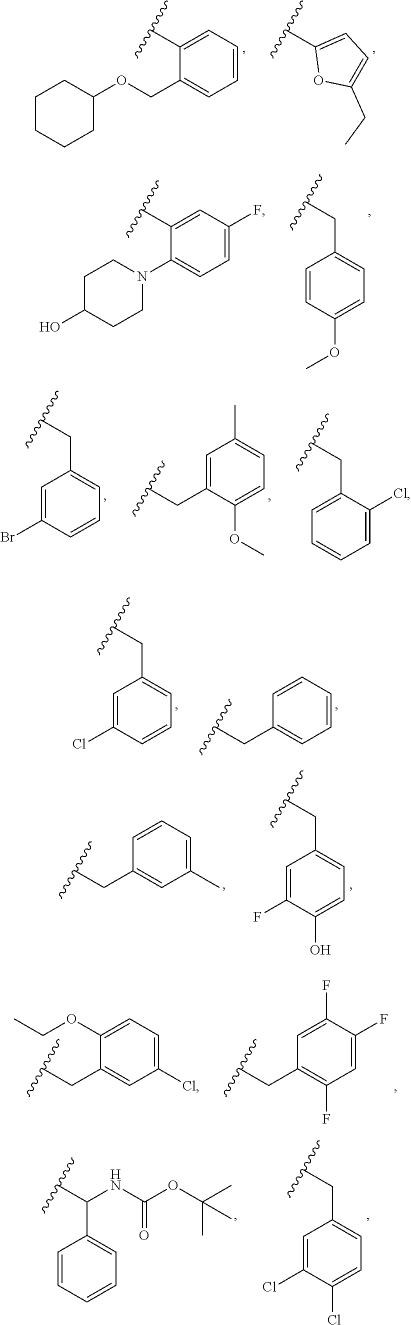

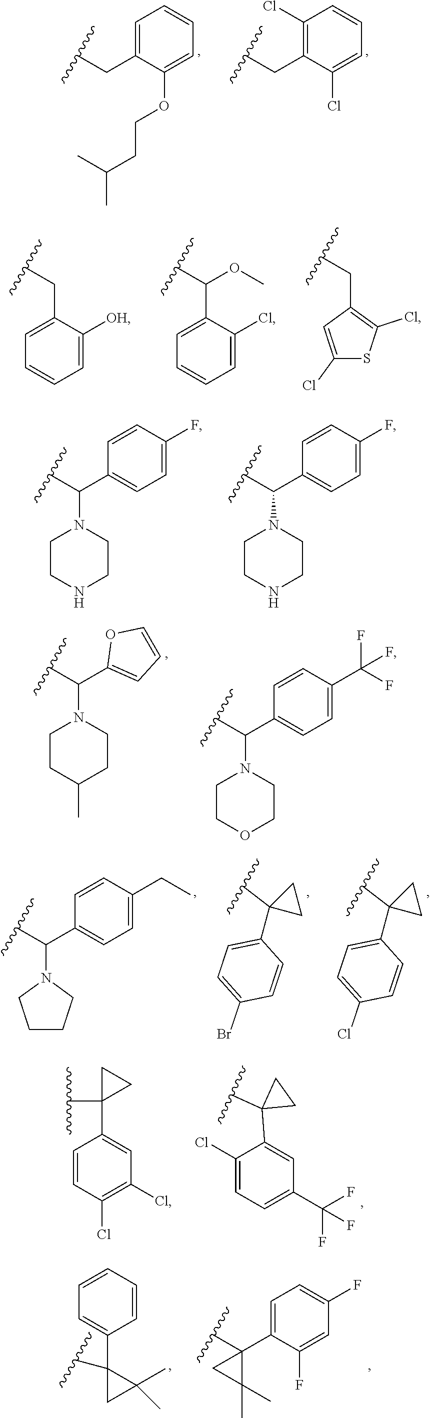

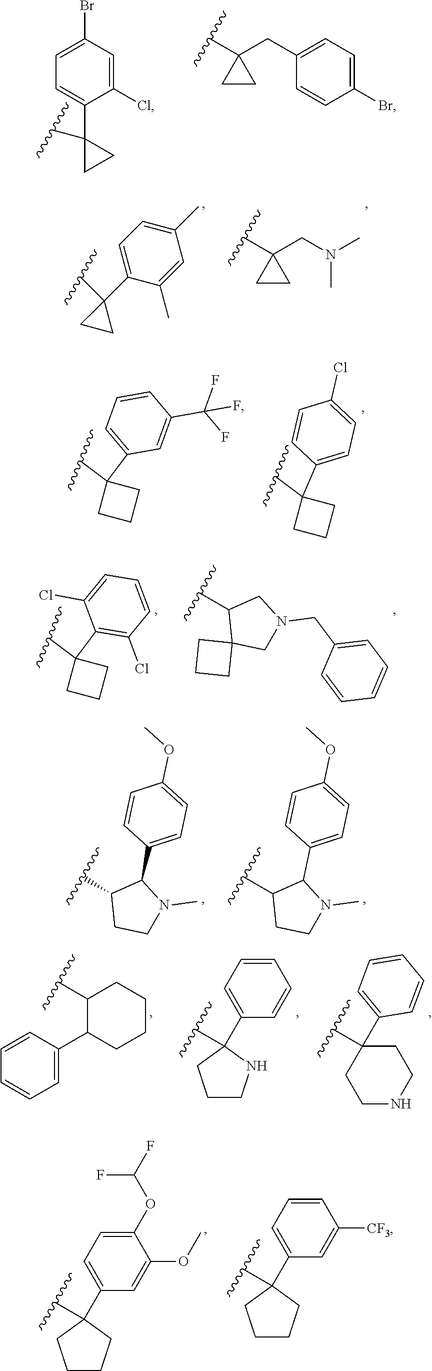

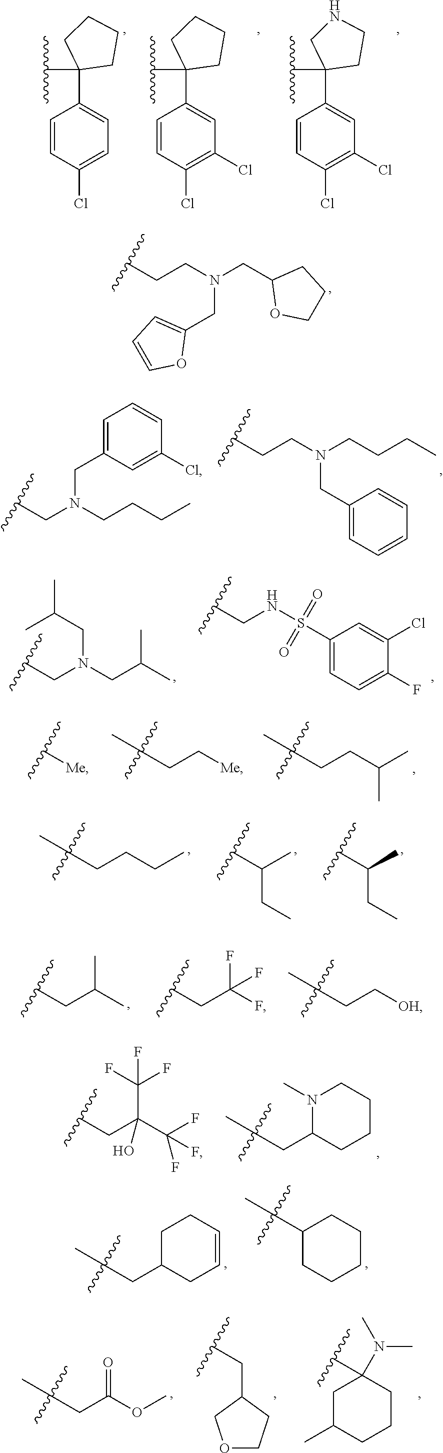

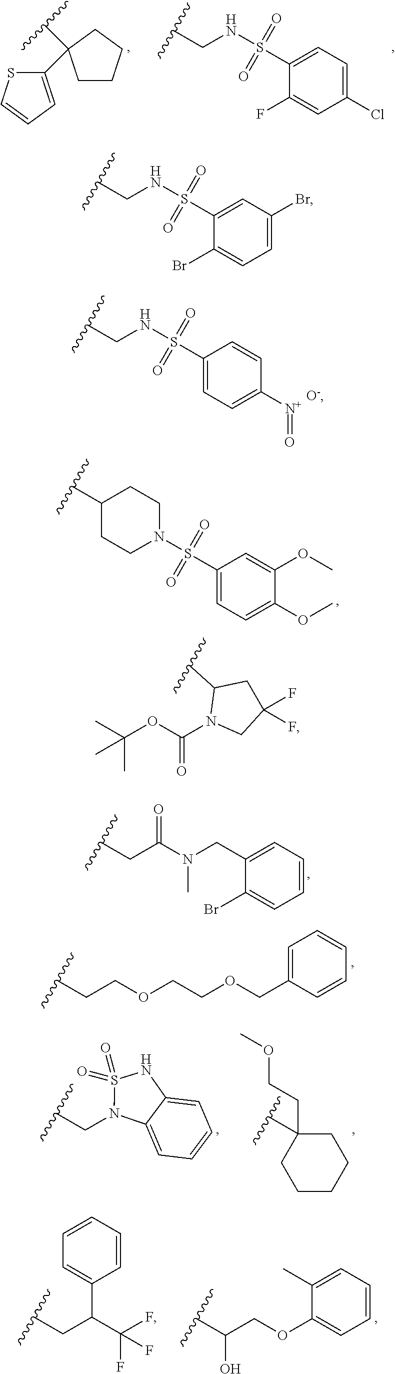

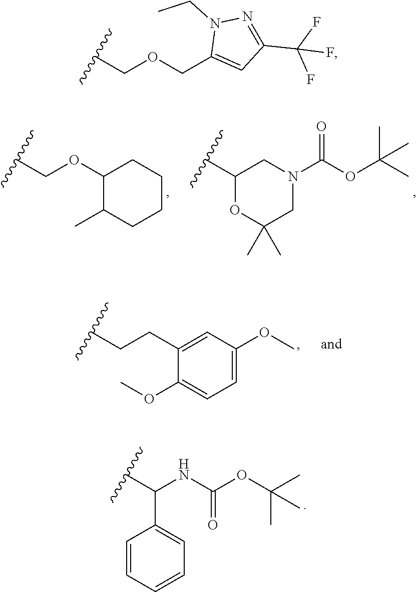

19. The compound of claim 15, wherein R³ is selected from the group consisting of:
methyl, ethyl, n-propyl, i-propyl, t-butyl,

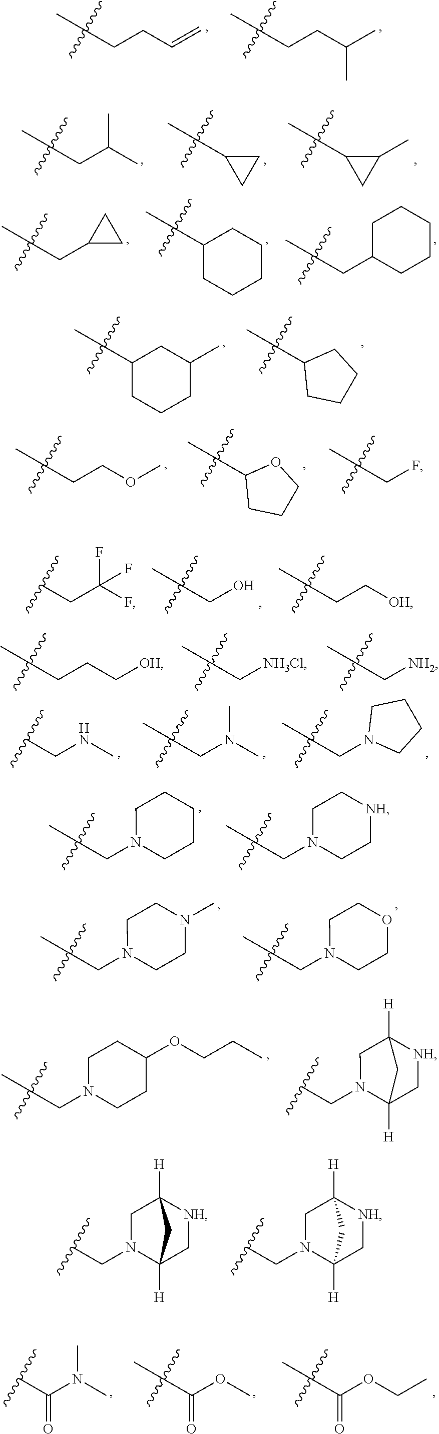

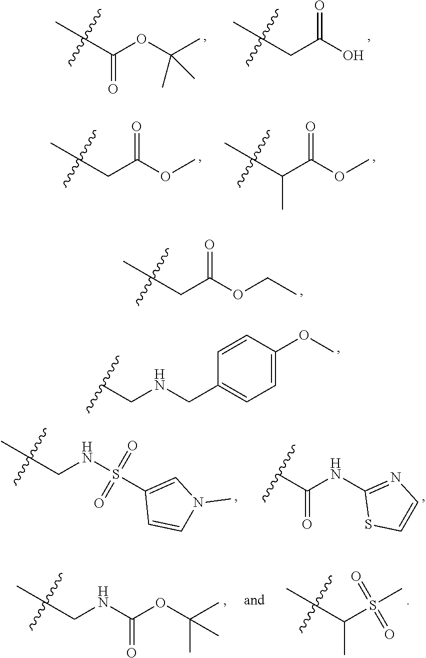

20. The compound of claim 15, wherein R⁴ is selected from the group consisting of methyl, ethyl, n-propyl, and

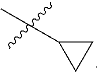 [SMILES: *C1CC1]

21. The compound of claim 15, wherein the compound of formula (II) is a compound of formula (IIa):

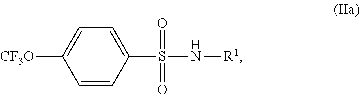

22. The compound of claim 21, wherein R¹ is selected from the group consisting of:

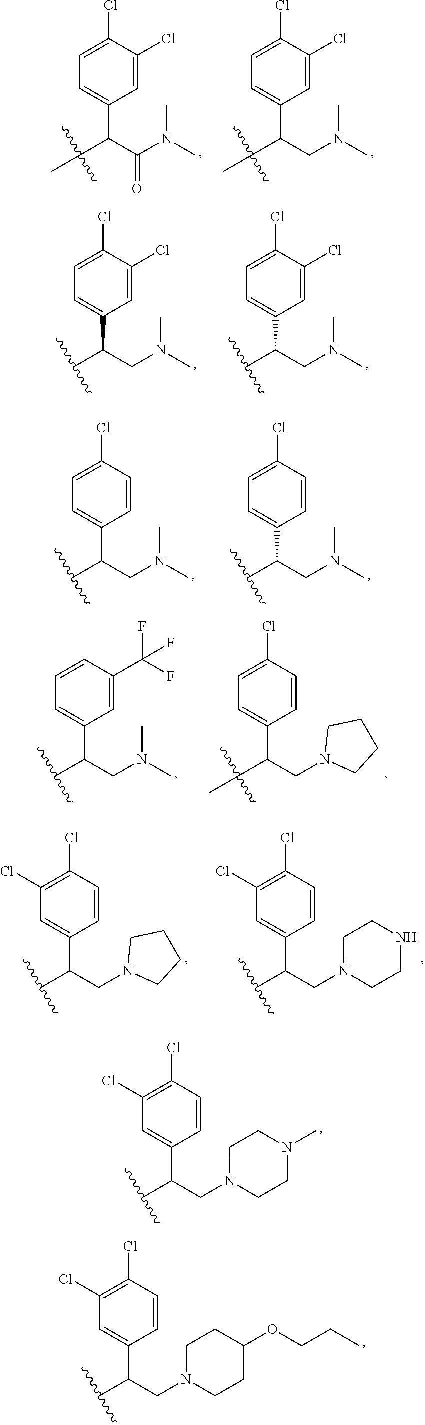

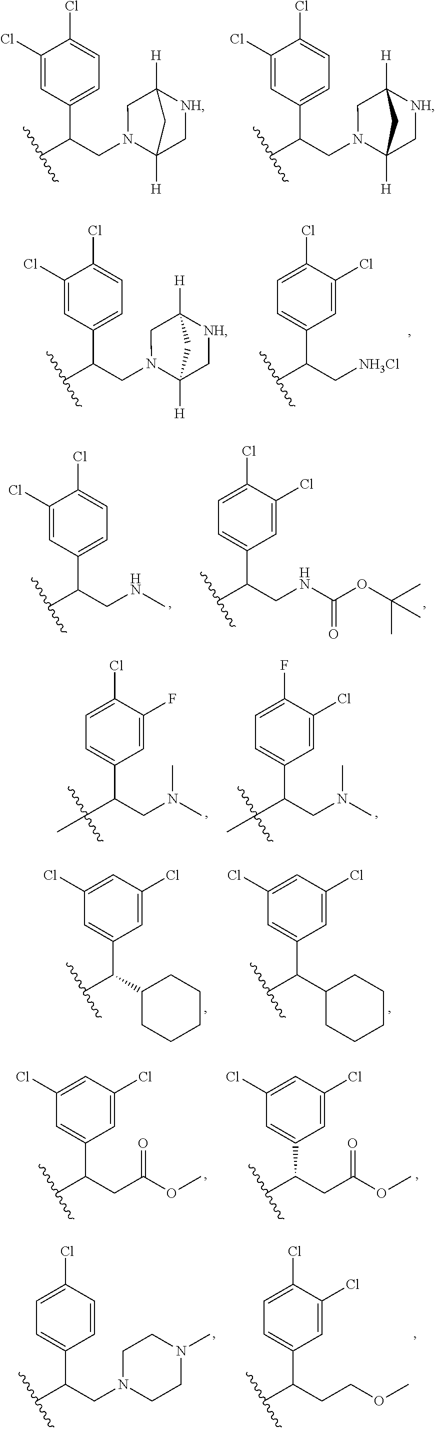

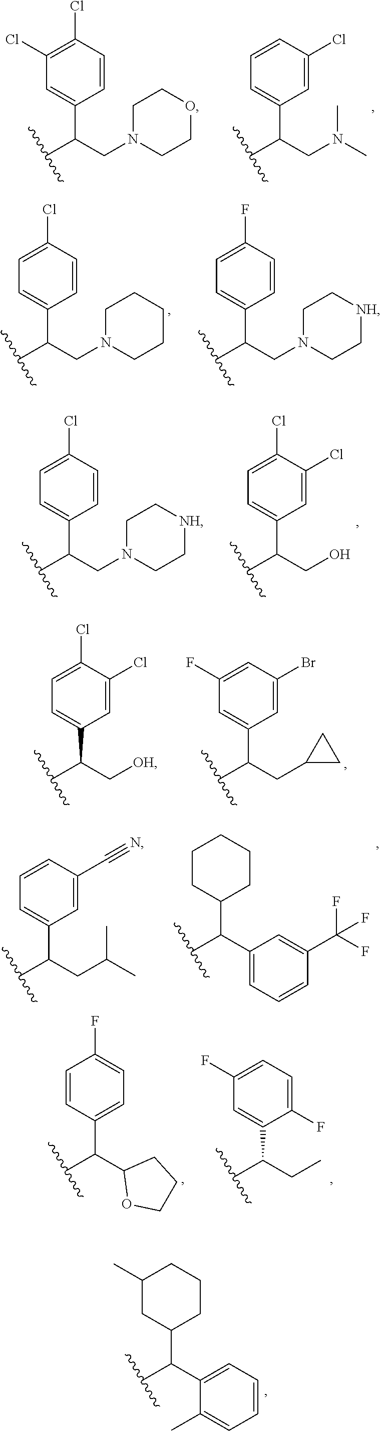

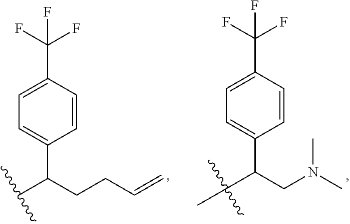 [SMILES: *C(CCC=C)c1ccc(C(F)(F)F)cc1 ; *C(CN(C)C)c1ccc(C(F)(F)F)cc1]

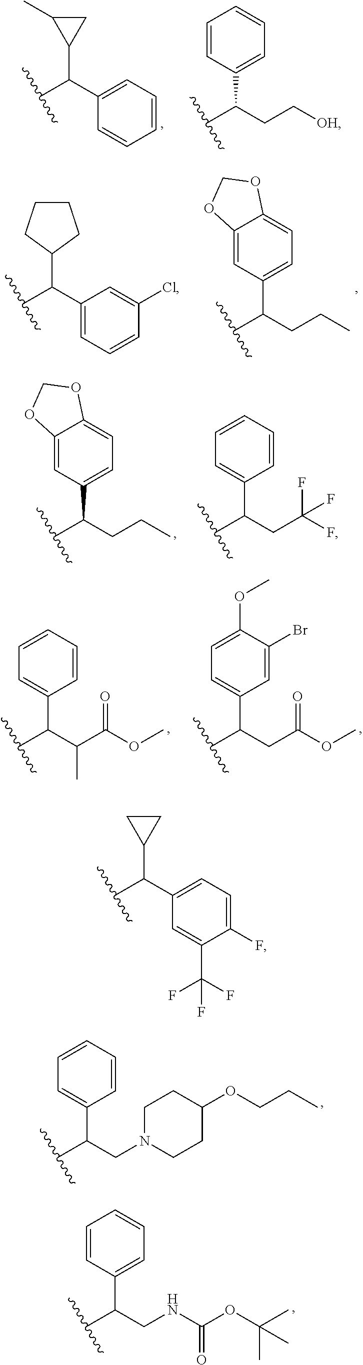

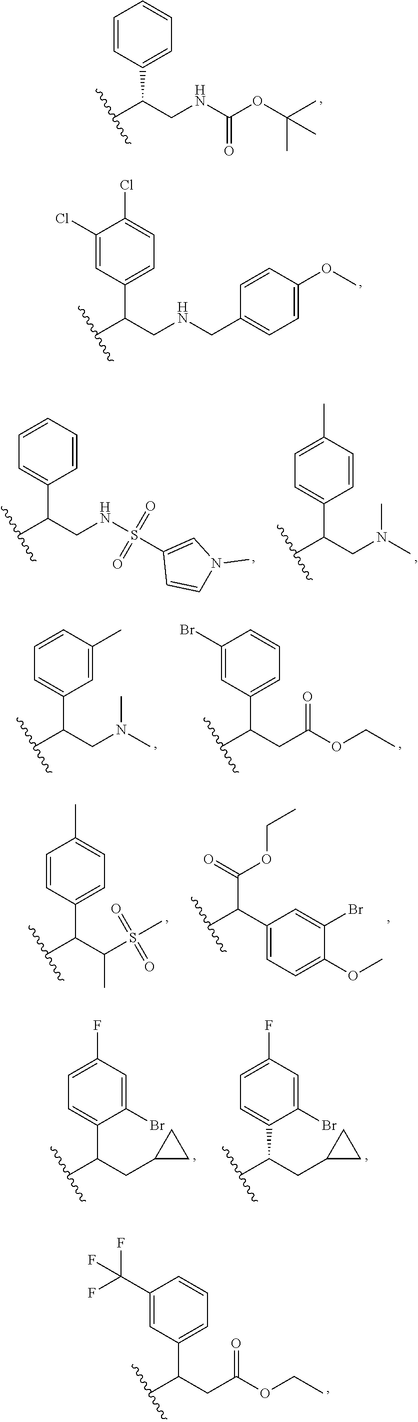

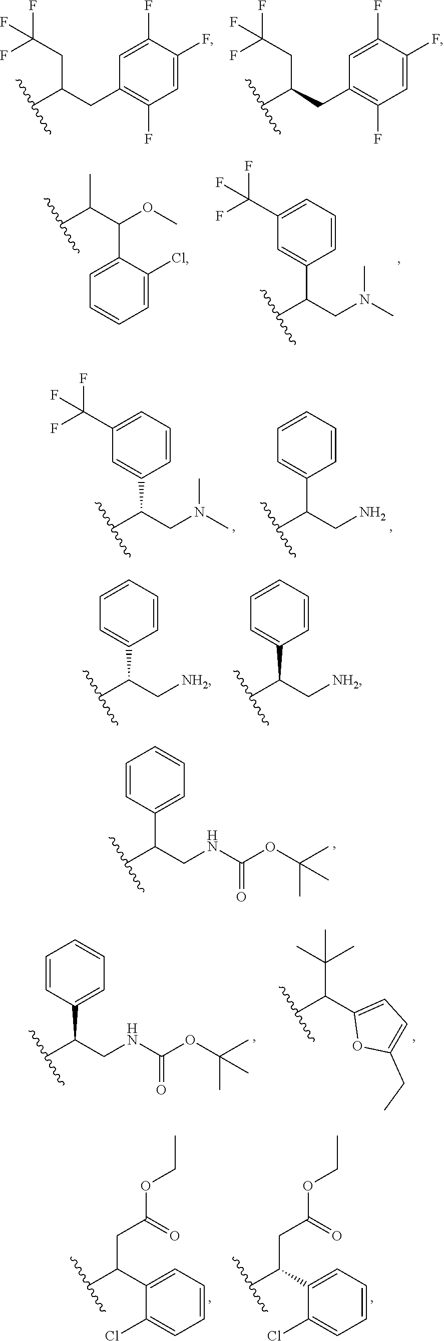

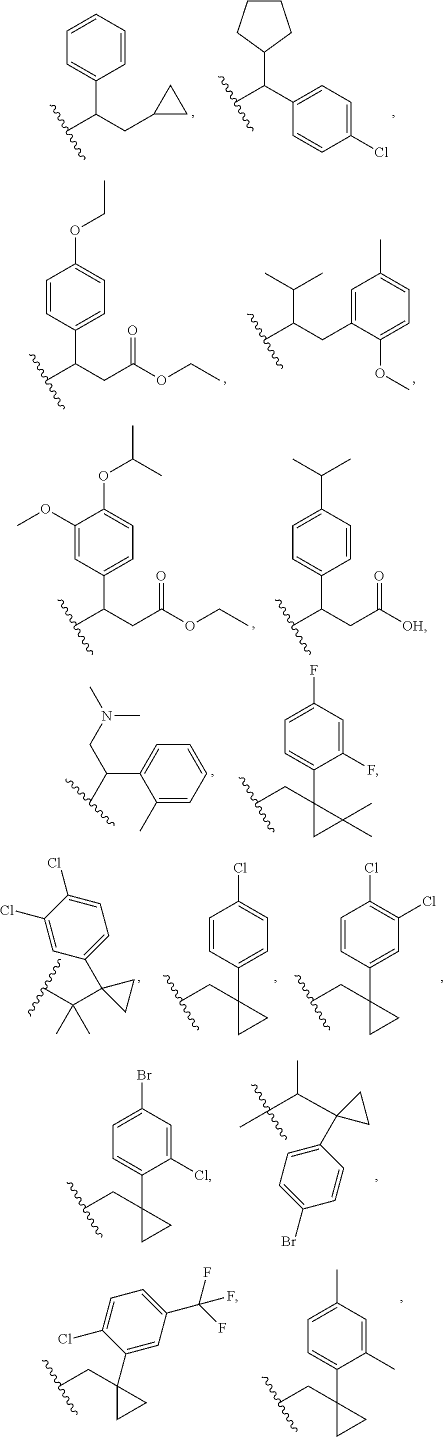

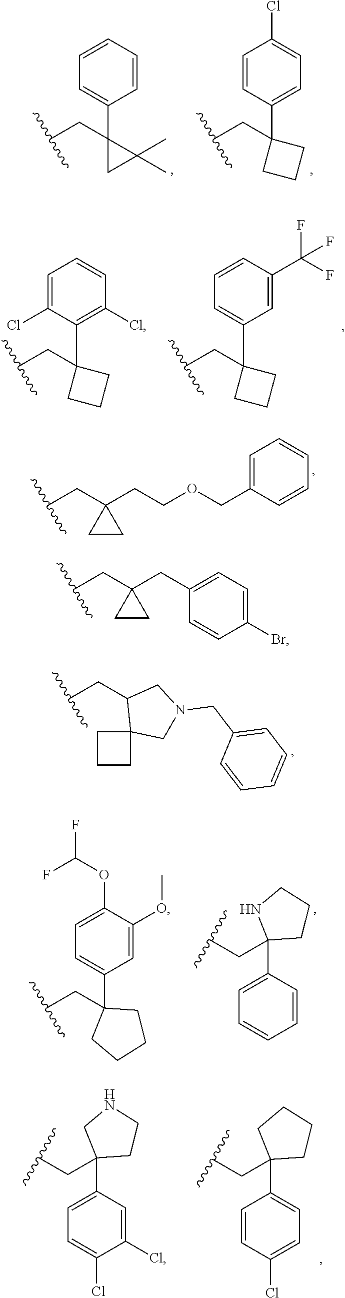

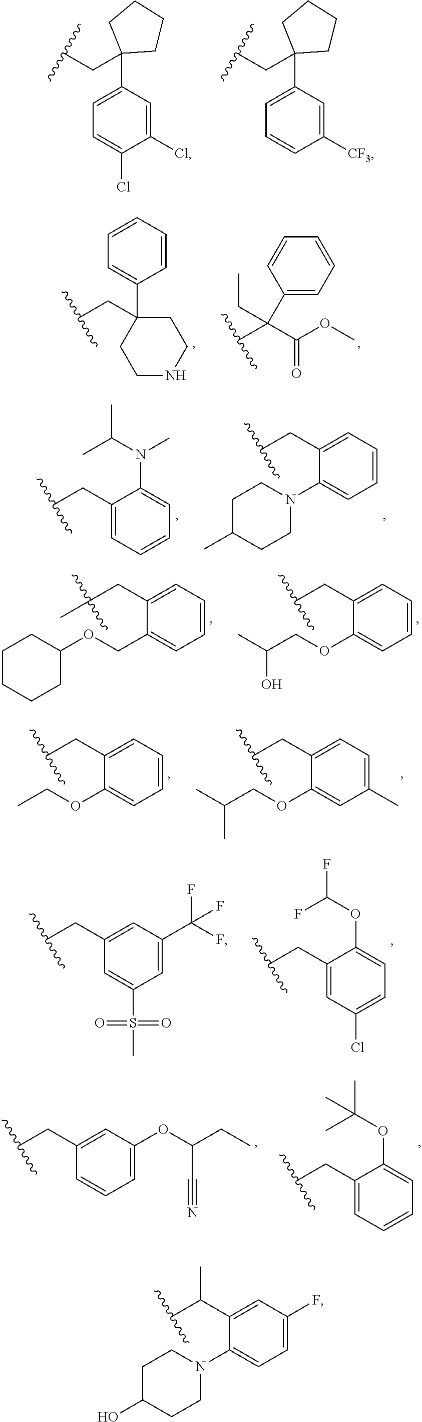

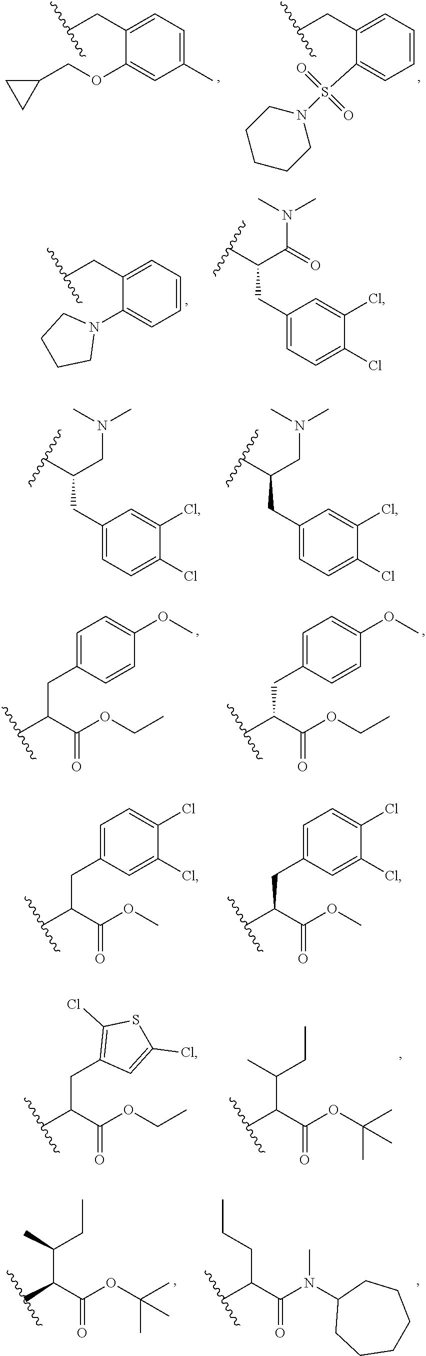

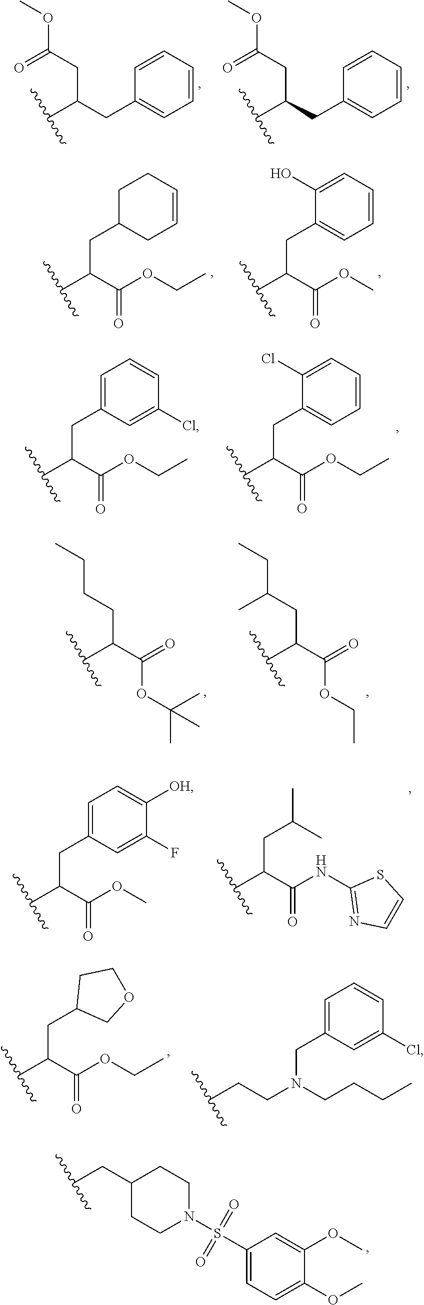

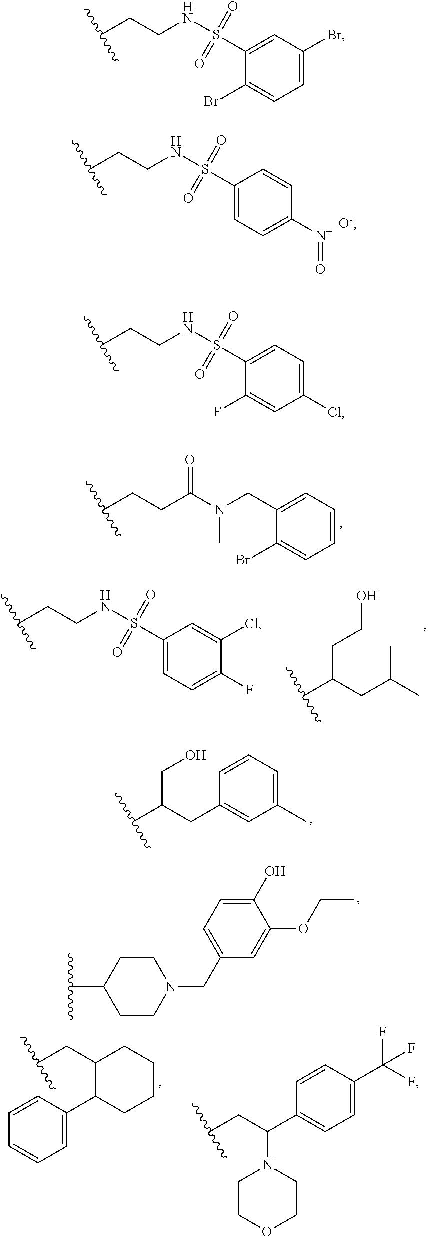

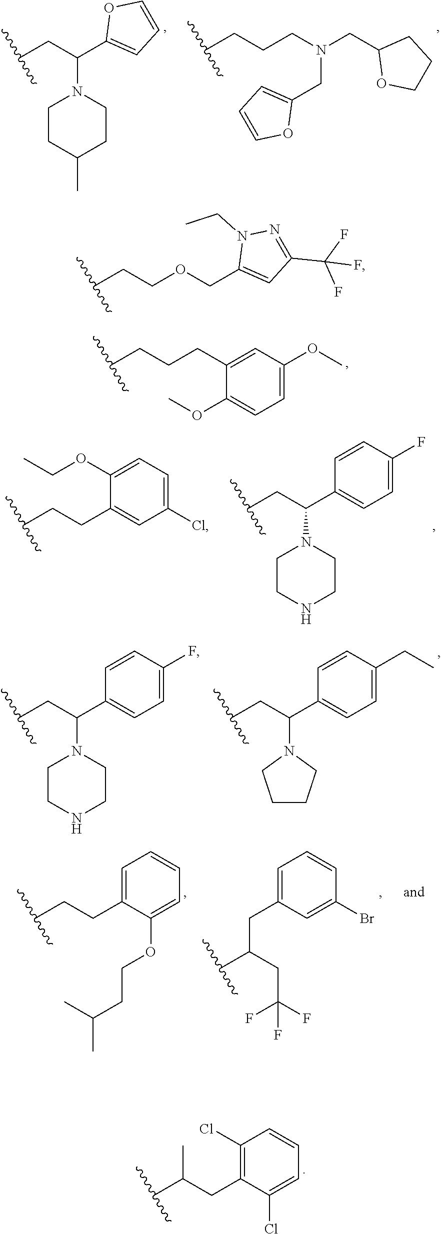

23. The compound of claim 15, wherein the compound of formula (II) is a compound of formula (IIb): 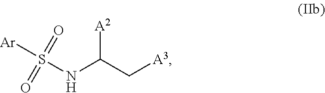
wherein:
A² is selected from the group consisting of 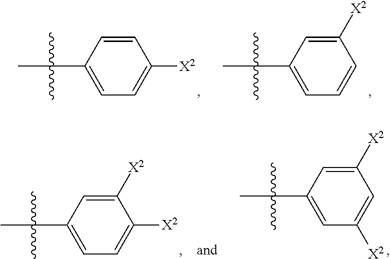
wherein each occurrence of X² is independently selected from the group consisting of H, F, Cl, Br, Me, and CF₃; and
A³ is selected from the group consisting of NMe₂, NH₂, NHMe, NHC(═O)Ot-Bu,

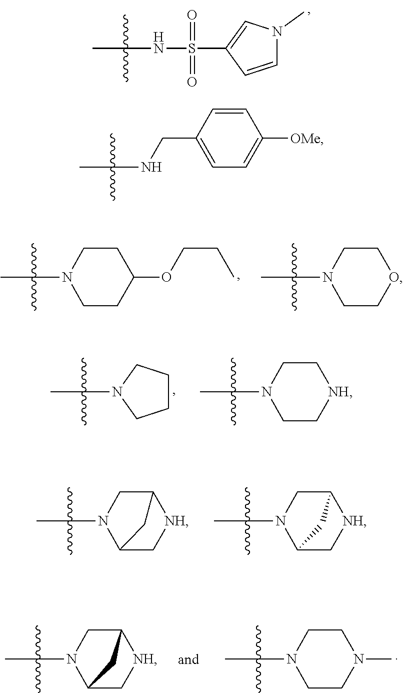

24. The compound of claim 23, wherein A² is

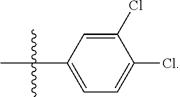 [SMILES: *c1ccc(Cl)c(Cl)c1]

25. The compound of claim 23, wherein A³ is NMe₂.

26. The compound of claim 23, wherein Ar is selected from the group consisting of:

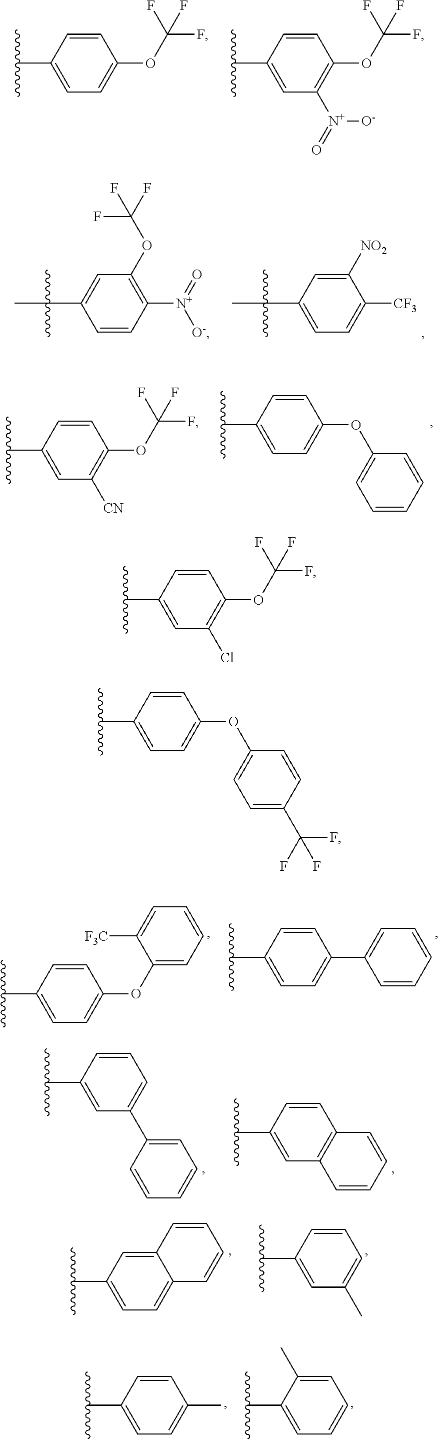

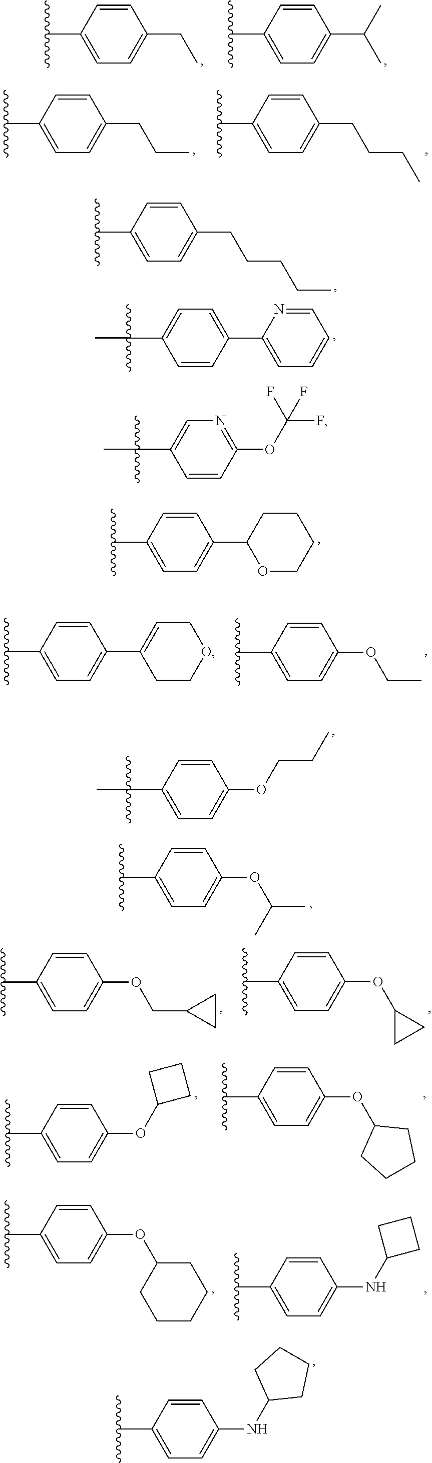

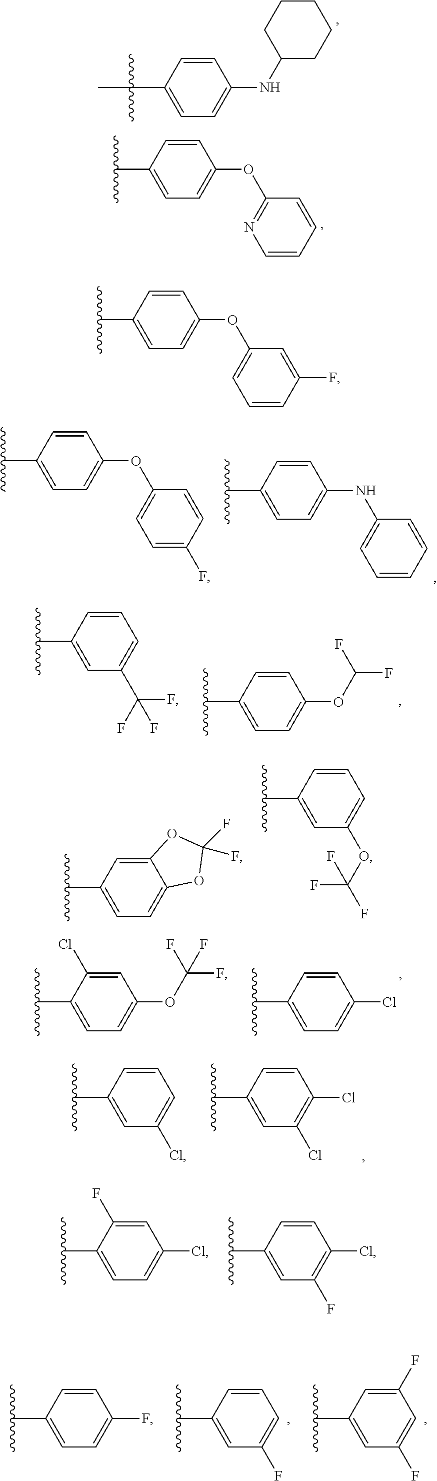

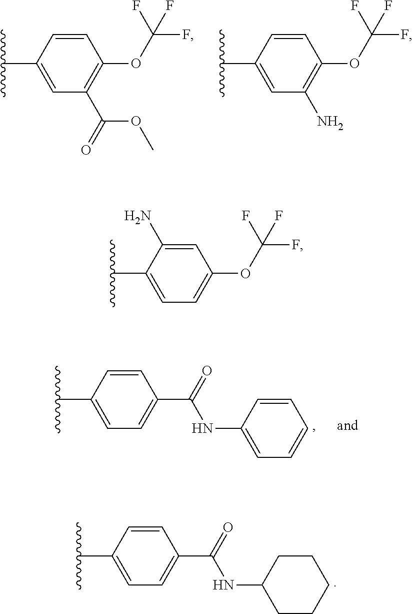

27. The compound of claim 1, which is selected from the group consisting of:
N-(1-(3,4-dichlorophenyl)-2-(dimethylamino)ethyl)-3-nitro-4-(trifluoromethoxy)benzenesulfonamide;
N-(1-hydroxy-5-methylhexan-3-yl)-4-(trifluoromethoxy)benzenesulfonamide;
N-(1-(3,4-dichlorophenyl)-2-(dimethylamino)ethyl)-4-pentylbenzenesulfonamide;
N-(cyclohexyl(3,5-dichlorophenyl)methyl)-4-(trifluoromethoxy)benzenesulfonamide;
N-(1-(3,4-dichlorophenyl)-2-(dimethylamino)ethyl)-4-(4-(trifluoromethyl)phenoxy)benzenesulfonamide;
N-(1-(3,4-dichlorophenyl)-2-(dimethylamino)ethyl)-4-phenoxybenzenesulfonamide;
N-(1-(3,4-dichlorophenyl)-2-(dimethylamino)ethyl)-[1,1′-biphenyl]-4-sulfonamide;
N-(1-(4-chlorophenyl)-2-(dimethylamino)ethyl)-4-phenoxybenzenesulfonamide;
N-(2-(dimethylamino)-1-(3-(trifluoromethyl)phenyl)ethyl)-4-phenoxybenzenesulfonamide;
3-chloro-N-(1-(3,4-dichlorophenyl)-2-(dimethylamino)ethyl)-4-(trifluoromethoxy)benzenesulfonamide;
4-(cyclopentyloxy)-N-(1-(3,4-dichlorophenyl)-2-(dimethylamino)ethyl)benzenesulfonamide;
N-(1-(3,4-dichlorophenyl)-2-(dimethylamino)ethyl)-4-propylbenzenesulfonamide;
N-(1-(3,4-dichlorophenyl)-2-(4-methylpiperazin-1-yl)ethyl)-4-(trifluoromethoxy)benzenesulfonamide;
N-(1-(3,4-dichlorophenyl)-2-(dimethylamino)ethyl)-[1,1′-biphenyl]-3-sulfonamide;
3-chloro-N-(1-(4-chlorophenyl)-2-(dimethylamino)ethyl)-4-(trifluoromethoxy)benzenesulfonamide;
3-chloro-N-(2-(dimethylamino)-1-(3-(trifluoromethyl)phenyl)ethyl)-4-(trifluoromethoxy)benzenesulfonamide;
N-(1-(3-bromo-5-fluorophenyl)-2-cyclopropylethyl)-4-(trifluoromethoxy)benzenesulfonamide;
N-((1-(2,4-difluorophenyl)-2,2-dimethylcyclopropyl)methyl)-4-(trifluoromethoxy)benzenesulfonamide;
N-(1-(3,4-dichlorophenyl)-2-(dimethylamino)ethyl)-4-(trifluoromethoxy)benzenesulfonamide;
N-(2-(dimethylamino)-1-(3-(trifluoromethyl)phenyl)ethyl)-4-(4-(trifluoromethyl)phenoxy)benzenesulfonamide;
N-(2-(isopropyl(methyl)amino)benzyl)-4-(trifluoromethoxy)benzenesulfonamide;
4-(trifluoromethoxy)-N-(1-(4-(trifluoromethyl)phenyl)pent-4-en-1-yl)benzenesulfonamide;
N-(1-(3,4-dichlorophenyl)-2-(dimethylamino)ethyl)-3-(trifluoromethoxy)benzenesulfonamide;
2-amino-N-(1-(3,4-dichlorophenyl)-2-(dimethylamino)ethyl)-4-(trifluoromethoxy)benzenesulfonamide;
N-(1-(3,4-dichlorophenyl)-2-(dimethylamino)ethyl)naphthalene-1-sulfonamide;
N-(2-((cyclohexyloxy)methyl)benzyl)-4-(trifluoromethoxy)benzenesulfonamide;
N-((1-(4-bromo-2-chlorophenyl)cyclopropyl)methyl)-4-(trifluoromethoxy)benzenesulfonamide;
N-(2-(4-methylpiperidin-1-yl)benzyl)-4-(trifluoromethoxy)benzenesulfonamide;
N-(1-(4-chlorophenyl)-2-(4-methylpiperazin-1-yl)ethyl)-4-(trifluoromethoxy)benzenesulfonamide;
2-chloro-N-(1-(3,4-dichlorophenyl)-2-(dimethylamino)ethyl)-4-(trifluoromethoxy)benzenesulfonamide;
N-(1-(3,4-dichlorophenyl)-2-(dimethylamino)ethyl)naphthalene-2-sulfonamide;
3,4-dichloro-N-(1-(3,4-dichlorophenyl)-2-(dimethylamino)ethyl)benzenesulfonamide;
N-((2-(4-methoxyphenyl)-1-methylpyrrolidin-3-yl)methyl)-4-(trifluoromethoxy)benzenesulfonamide;
N-(1-hydroxy-3-(m-tolyl)propan-2-yl)-4-(trifluoromethoxy)benzenesulfonamide;
N-(1-(3,4-dichlorophenyl)-2-(dimethylamino)ethyl)-2-phenylethane-1-sulfonamide;
N-(1-(3,4-dichlorophenyl)-2-(pyrrolidin-1-yl)ethyl)-4-(trifluoromethoxy)benzenesulfonamide;
2,5-dibromo-N-(2-((4-(trifluoromethoxy)phenyl)sulfonamido)ethyl)benzenesulfonamide;
N-(1-(3,4-dichlorophenyl)-2-(dimethylamino)ethyl)-N′-methyl-4-(trifluoromethoxy)benzenesulfonimidamide;
N-(1-(3,4-dichlorophenyl)-2-(dimethylamino)ethyl)-2,2-difluorobenzo[d][1,3]dioxole-5-sulfonamide;
ethyl 3-((4-(trifluoromethoxy)phenyl)sulfonamido)-3-(3-(trifluoromethyl)phenyl)propanoate;
N-(1-(1-(4-bromophenyl)cyclopropyl)ethyl)-4-(trifluoromethoxy)benzenesulfonamide;
N-((1-(2-chloro-5-(trifluoromethyl)phenyl)cyclopropyl)methyl)-4-(trifluoromethoxy)benzenesulfonamide;
4-methyl-N-(thiazol-2-yl)-2-((4-(trifluoromethoxy)phenyl)sulfonamido)pentanamide;
N-(1-(3,4-dichlorophenyl)-2-(dimethylamino)ethyl)-4-isopropylbenzenesulfonamide;
N-(1-(3,4-dichlorophenyl)-2-(dimethylamino)ethyl)-4-(trifluoromethyl)benzenesulfonamide;
N-(4,4,4-trifluoro-1-(2,4,5-trifluorophenyl)butan-2-yl)-4-(trifluoromethoxy)benzenesulfonamide;
N-((6-benzyl-6-azaspiro[3.4]octan-8-yl)methyl)-4-(trifluoromethoxy)benzenesulfonamide;
N-(2-((1-ethyl-3-(trifluoromethyl)-1H-pyrazol-5-yl)methoxy)ethyl)-4-(trifluoromethoxy)benzenesulfonamide;
4-chloro-N-(1-(3,4-dichlorophenyl)-2-(dimethylamino)ethyl)benzenesulfonamide;
N-(1-(3-cyanophenyl)-3-methylbutyl)-4-(trifluoromethoxy)benzenesulfonamide;
N-(cyclohexyl(3-(trifluoromethyl)phenyl)methyl)-4-(trifluoromethoxy)benzenesulfonamide;
N-((1-(2,6-dichlorophenyl)cyclobutyl)methyl)-4-(trifluoromethoxy)benzenesulfonamide;
N-(2-(cyclopropylmethoxy)-4-methylbenzyl)-4-(trifluoromethoxy)benzenesulfonamide;
(E)-N-(1-(3,4-dichlorophenyl)-2-(dimethylamino)ethyl)-2-phenylethene-1-sulfonamide;
N-(2-(2-hydroxypropoxy)benzyl)-4-(trifluoromethoxy)benzenesulfonamide;
3-chloro-4-fluoro-N-(2-((4-(trifluoromethoxy)phenyl)sulfonamido)ethyl)benzenesulfonamide;
3-amino-N-(1-(3,4-dichlorophenyl)-2-(dimethylamino)ethyl)-4-(trifluoromethoxy)benzenesulfonamide;
N-(2-(butyl(3-chlorobenzyl)amino)ethyl)-4-(trifluoromethoxy)benzenesulfonamide;
N-((4-fluorophenyl)(tetrahydrofuran-2-yl)methyl)-4-(trifluoromethoxy)benzenesulfonamide;
N-(1-(4-chlorophenyl)-2-(piperidin-1-yl)ethyl)-4-(trifluoromethoxy)benzenesulfonamide;
N-(1-(3,4-dichlorophenyl)-2-(dimethylamino)ethyl)-4-ethylbenzenesulfonamide;
N-(1-(2,5-difluorophenyl)propyl)-4-(trifluoromethoxy)benzenesulfonamide;
methyl 3-(3,5-dichlorophenyl)-3-((4-(trifluoromethoxy)phenyl)sulfonamido)propanoate;
N-((3-chlorophenyl)(cyclopentyl)methyl)-4-(trifluoromethoxy)benzenesulfonamide;
4-(trifluoromethoxy)-N-((1-(3-(trifluoromethyl)phenyl)cyclobutyl)methyl)benzenesulfonamide;
N-(1-(benzo[d][1,3]dioxol-5-yl)butyl)-4-(trifluoromethoxy)benzenesulfonamide;
methyl 3-(3-fluoro-4-hydroxyphenyl)-2-((4-(trifluoromethoxy)phenyl)sulfonamido)propanoate;
N-(5-chloro-2-ethoxyphenethyl)-4-(trifluoromethoxy)benzenesulfonamide;
N-((2-phenylcyclohexyl)methyl)-4-(trifluoromethoxy)benzenesulfonamide;
methyl 3-(3-bromo-4-methoxyphenyl)-3-((4-(trifluoromethoxy)phenyl)sulfonamido)propanoate;
tert-butyl 2-((4-(trifluoromethoxy)phenyl)sulfonamido)hexanoate;
N-((1-(4-bromobenzyl)cyclopropyl)methyl)-4-(trifluoromethoxy)benzenesulfonamide;
N-(cyclopropyl(4-fluoro-3-(trifluoromethyl)phenyl)methyl)-4-(trifluoromethoxy)benzenesulfonamide;
N-(1-(3,4-dichlorophenyl)-2-hydroxyethyl)-4-(trifluoromethoxy)benzenesulfonamide;
N-(3-(2,5-dimethoxyphenyl)propyl)-4-(trifluoromethoxy)benzenesulfonamide;
N-(1-(3,4-dichlorophenyl)-2-(dimethylamino)ethyl)-3-methylbenzenesulfonamide;
4-chloro-2-fluoro-N-(2-((4-(trifluoromethoxy)phenyl)sulfonamido)ethyl)benzenesulfonamide;
methyl 2-(2-bromophenyl)-2-((4-(trifluoromethoxy)phenyl)sulfonamido)acetate;
tert-butyl 4,4-difluoro-2-(((4-(trifluoromethoxy)phenyl)sulfonamido)methyl)pyrrolidine-1-carboxylate;
N-(2-bromobenzyl)-N-methyl-3-((4-(trifluoromethoxy)phenyl)sulfonamido)propanamide;
N-((1-(2,4-dimethylphenyl)cyclopropyl)methyl)-4-(trifluoromethoxy)benzenesulfonamide;
N-(1-(3-chlorophenyl)-2-(dimethylamino)ethyl)-4-(trifluoromethoxy)benzenesulfonamide;
N-(2-morpholino-2-(4-(trifluoromethyl)phenyl)ethyl)-4-(trifluoromethoxy)benzenesulfonamide;
N-(2-(dimethylamino)-1-(p-tolyl)ethyl)-4-(trifluoromethoxy)benzenesulfonamide;
N-(1-(3,4-dichlorophenyl)-2-(dimethylamino)ethyl)-4-(difluoromethoxy)benzenesulfonamide;
N-(2-(furan-2-yl)-2-(4-methylpiperidin-1-yl)ethyl)-4-(trifluoromethoxy)benzenesulfonamide;
ethyl 3-(2-chlorophenyl)-2-((4-(trifluoromethoxy)phenyl)sulfonamido)propanoate;
N-(2-(tert-butoxy)benzyl)-4-(trifluoromethoxy)benzenesulfonamide;
3-chloro-N-(1-(3,4-dichlorophenyl)-2-(dimethylamino)ethyl)benzenesulfonamide;
N-(2-(dimethylamino)-1-(3-(trifluoromethyl)phenyl)ethyl)-4-(trifluoromethoxy)benzenesulfonamide;
N-(1-(3,4-dichlorophenyl)-2-(dimethylamino)ethyl)-4-(trifluoromethoxy)benzenesulfonimidamide;
N-(1-(5-fluoro-2-(4-hydroxypiperidin-1-yl)phenyl)ethyl)-4-(trifluoromethoxy)benzenesulfonamide;
N-(2-(dimethylamino)-1-(m-tolyl)ethyl)-4-(trifluoromethoxy)benzenesulfonamide;
ethyl 3-(3-bromophenyl)-3-((4-(trifluoromethoxy)phenyl)sulfonamido)propanoate;
N-(1-(3,4-dichlorophenyl)-2-(dimethylamino)ethyl)-2-methylbenzenesulfonamide;
4-chloro-N-(1-(3,4-dichlorophenyl)-2-(dimethylamino)ethyl)-3-fluorobenzenesulfonamide;
ethyl 3-(3-chlorophenyl)-2-((4-(trifluoromethoxy)phenyl)sulfonamido)propanoate;
N-(1-(2-bromo-4-fluorophenyl)-2-cyclopropylethyl)-4-(trifluoromethoxy)benzenesulfonamide;
N-(2-(pyrrolidin-1-yl)benzyl)-4-(trifluoromethoxy)benzenesulfonamide;
ethyl 2-(3-bromo-4-methoxyphenyl)-2-((4-(trifluoromethoxy)phenyl)sulfonamido)acetate;
methyl 3-(2-hydroxyphenyl)-2-((4-(trifluoromethoxy)phenyl)sulfonamido)propanoate;
N-(1-(3,4-dichlorophenyl)-3-methoxypropyl)-4-(trifluoromethoxy)benzenesulfonamide;
N-(1-(2-chlorophenyl)-1-methoxypropan-2-yl)-4-(trifluoromethoxy)benzenesulfonamide;
N-(1-(3,4-dichlorophenyl)-2-(dimethylamino)ethyl)-4-ethoxybenzenesulfonamide;
N-(1-(4-chlorophenyl)-2-(pyrrolidin-1-yl)ethyl)-4-(trifluoromethoxy)benzenesulfonamide;
N-(1-(3,4-dichlorophenyl)-2-(dimethylamino)ethyl)-4-methylbenzenesulfonamide;
N′-(1-(3,4-dichlorophenyl)-2-(dimethylamino)ethyl)-N-methyl-4-(trifluoromethoxy)benzenesulfonimidamide;
N-(1-(3,4-dichlorophenyl)-2-(dimethylamino)ethyl)-3-(trifluoromethyl)benzenesulfonamide;
ethyl 3-(cyclohex-3-en-1-yl)-2-((4-(trifluoromethoxy)phenyl)sulfonamido)propanoate;
N-cyclohexyl-4-(N-(1-(3,4-dichlorophenyl)-2-(dimethylamino)ethyl)sulfamoyl)benzamide;
N-(1-(3,4-dichlorophenyl)-2-morpholinoethyl)-4-(trifluoromethoxy)benzenesulfonamide;
N-cycloheptyl-N-methyl-2-((4-(trifluoromethoxy)phenyl)sulfonamido)pentanamide;
N-(4,4,4-trifluoro-3-phenylbutyl)-4-(trifluoromethoxy)benzenesulfonamide;
N-(1-(5-ethylfuran-2-yl)-2,2-dimethylpropyl)-4-(trifluoromethoxy)benzenesulfonamide;
N-(2-(4-ethylphenyl)-2-(pyrrolidin-1-yl)ethyl)-4-(trifluoromethoxy)benzenesulfonamide;
N-(2-(isopentyloxy)phenethyl)-4-(trifluoromethoxy)benzenesulfonamide;
N-(1-(4-chlorophenyl)-2-(dimethylamino)ethyl)-4-(trifluoromethoxy)benzenesulfonamide;
methyl 3-(3,4-dichlorophenyl)-2-((4-(trifluoromethoxy)phenyl)sulfonamido)propanoate;
N-(1-(3,4-dichlorophenyl)-2-(dimethylamino)ethyl)cyclohexanesulfonamide;
ethyl 3-(2,5-dichlorothiophen-3-yl)-2-((4-(trifluoromethoxy)phenyl)sulfonamido)propanoate;
tert-butyl 3-methyl-2-((4-(trifluoromethoxy)phenyl)sulfonamido)pentanoate;
N-(2-(piperidin-1-ylsulfonyl)benzyl)-4-(trifluoromethoxy)benzenesulfonamide;
4-chloro-N-(1-(3,4-dichlorophenyl)-2-(dimethylamino)ethyl)-2-fluorobenzenesulfonamide;
N-(1-(3-bromophenyl)-4,4,4-trifluorobutan-2-yl)-4-(trifluoromethoxy)benzenesulfonamide;
N-(2-(dimethylamino)-1-(o-tolyl)ethyl)-4-(trifluoromethoxy)benzenesulfonamide;
N-(2-ethoxybenzyl)-4-(trifluoromethoxy)benzenesulfonamide;
N-(2-isobutoxy-4-methylbenzyl)-4-(trifluoromethoxy)benzenesulfonamide;
ethyl 3-(tetrahydrofuran-3-yl)-2-((4-(trifluoromethoxy)phenyl)sulfonamido)propanoate;
methyl 4-phenyl-3-((4-(trifluoromethoxy)phenyl)sulfonamido)butanoate;
N-(1-(3,4-dichlorophenyl)-2-(dimethylamino)ethyl)-4-fluorobenzenesulfonamide;
N-((1-((3,4-dimethoxyphenyl)sulfonyl)piperidin-4-yl)methyl)-4-(trifluoromethoxy)benzenesulfonamide;
N-(3-(methylsulfonyl)-5-(trifluoromethyl)benzyl)-4-(trifluoromethoxy)benzenesulfonamide;
4-nitro-N-(2-((4-(trifluoromethoxy)phenyl)sulfonamido)ethyl)benzenesulfonamide;
3-(4-isopropylphenyl)-3-((4-(trifluoromethoxy)phenyl)sulfonamido)propanoic acid;
methyl 2-phenyl-2-((4-(trifluoromethoxy)phenyl)sulfonamido)butanoate;
N-(1-(3,4-dichlorophenyl)-2-(dimethylamino)ethyl)-3-fluorobenzenesulfonamide;
N-(5-chloro-2-(difluoromethoxy)benzyl)-4-(trifluoromethoxy)benzenesulfonamide;
ethyl 4-methyl-2-((4-(trifluoromethoxy)phenyl)sulfonamido)hexanoate;
N-(3-(1-cyanopropoxy)benzyl)-4-(trifluoromethoxy)benzenesulfonamide;
ethyl 3-(4-methoxyphenyl)-2-((4-(trifluoromethoxy)phenyl)sulfonamido)propanoate;
ethyl 3-(4-isopropoxy-3-methoxyphenyl)-3-((4-(trifluoromethoxy)phenyl)sulfonamido)propanoate;
N-((1-(4-(difluoromethoxy)-3-methoxyphenyl)cyclopentyl)methyl)-4-(trifluoromethoxy)benzenesulfonamide;
N-((4-chlorophenyl)(cyclopentyl)methyl)-4-(trifluoromethoxy)benzenesulfonamide;
ethyl 3-(4-ethoxyphenyl)-3-((4-(trifluoromethoxy)phenyl)sulfonamido)propanoate;
4-cyclopropoxy-N-(1-(3,4-dichlorophenyl)-2-(dimethylamino)ethyl)benzenesulfonamide;
N-(1-(3-chloro-4-fluorophenyl)-2-(dimethylamino)ethyl)-4-(trifluoromethoxy)benzenesulfonamide;
N-(1-(4-chloro-3-fluorophenyl)-2-(dimethylamino)ethyl)-4-(trifluoromethoxy)benzenesulfonamide;
4-(N-(1-(3,4-dichlorophenyl)-2-(dimethylamino)ethyl)sulfamoyl)-N-phenylbenzamide;
N-(1-(3,4-dichlorophenyl)-2-(dimethylamino)ethyl)-4-(phenylamino)benzenesulfonamide;
N-(1-(4-chlorophenyl)-2-(dimethylamino)ethyl)-4-(4-(trifluoromethyl)phenoxy)benzenesulfonamide;
N-(2-(dimethylamino)-1-(3-(trifluoromethyl)phenyl)ethyl)-3-nitro-4-(trifluoromethoxy)benzenesulfonamide;
N-(1-(4-chlorophenyl)-2-(dimethylamino)ethyl)-3-nitro-4-(trifluoromethoxy)benzenesulfonamide;
4-(cyclopropylmethoxy)-N-(1-(3,4-dichlorophenyl)-2-(dimethylamino)ethyl)benzenesulfonamide;
N-(1-(3,4-dichlorophenyl)-2-(dimethylamino)ethyl)-4-isopropoxybenzenesulfonamide;
4-(cyclohexyloxy)-N-(1-(3,4-dichlorophenyl)-2-(dimethylamino)ethyl)benzenesulfonamide;
N-(1-(3,4-dichlorophenyl)-2-(dimethylamino)ethyl)-4-(pyridin-2-yloxy)benzenesulfonamide;
4-butyl-N-(1-(3,4-dichlorophenyl)-2-(dimethylamino)ethyl)benzenesulfonamide;
N-(1-(3,4-dichlorophenyl)-2-(dimethylamino)ethyl)-4-(3,6-dihydro-2H-pyran-4-yl)benzenesulfonamide;
N-(2-(dimethylamino)-1-(4-(trifluoromethyl)phenyl)ethyl)-4-(trifluoromethoxy)benzenesulfonamide;
N-(1-(3,4-dichlorophenyl)-2-(dimethylamino)ethyl)-4-(3-fluorophenoxy)benzenesulfonamide;
N-(1-(3,4-dichlorophenyl)-2-(dimethylamino)ethyl)-4-(4-fluorophenoxy)benzenesulfonamide;
methyl 5-(N-(1-(3,4-dichlorophenyl)-2-(dimethylamino)ethyl)sulfamoyl)-2-(trifluoromethoxy)benzoate;
N-(1-(3,4-dichlorophenyl)-2-(4-methylpiperazin-1-yl)ethyl)-4-(4-(trifluoromethyl)phenoxy)benzenesulfonamide;
N-(1-(4-chlorophenyl)-2-(4-methylpiperazin-1-yl)ethyl)-4-phenoxybenzenesulfonamide;
N-(1-(4-chlorophenyl)-2-(4-methylpiperazin-1-yl)ethyl)-4-(4-(trifluoromethyl)phenoxy)benzenesulfonamide;
N-(1-(3,4-dichlorophenyl)-2-(4-methylpiperazin-1-yl)ethyl)-4-phenoxybenzenesulfonamide;
N-(2-(dimethylamino)-1-(3-(trifluoromethyl)phenyl)ethyl)-4-(4-(trifluoromethyl)phenoxy)benzenesulfonamide;
N-(1-(3,4-dichlorophenyl)-2-(dimethylamino)ethyl)-6-(trifluoromethoxy)pyridine-3-sulfonamide;
N-(1-(3,4-dichlorophenyl)-2-(dimethylamino)ethyl)-3-nitro-4-(trifluoromethoxy)benzenesulfonamide;
N-(1-(3,4-dichlorophenyl)-2-(dimethylamino)ethyl)-4-(pyridin-2-yl)benzenesulfonamide;
N-(1-(3,4-dichlorophenyl)-2-(dimethylamino)ethyl)-4-nitro-3-(trifluoromethoxy)benzenesulfonamide;
N-(2-amino-1-(3,4-dichlorophenyl)ethyl)-4-(trifluoromethoxy)benzenesulfonamide hydrochloride;
tert-butyl (2-(3,4-dichlorophenyl)-2-((4-(trifluoromethoxy)phenyl)sulfonamido)ethyl)carbamate;
N-((1-(4-chlorophenyl)cyclopropyl)methyl)-4-(trifluoromethoxy)benzenesulfonamide;
N-((1-(4-chlorophenyl)cyclobutyl)methyl)-4-(trifluoromethoxy)benzenesulfonamide;
N-((2,2-dimethyl-1-phenylcyclopropyl)methyl)-4-(trifluoromethoxy)benzenesulfonamide;
N-(1-(3,4-dichlorophenyl)-2-(dimethylamino)ethyl)-2-nitro-3-(trifluoromethoxy)benzenesulfonamide;
N-((1-(3,4-dichlorophenyl)cyclopropyl)methyl)-4-(trifluoromethoxy)benzenesulfonamide;
N-((1-(3,4-dichlorophenyl)cyclopropyl)methyl)-4-(4-(trifluoromethyl)phenoxy)benzenesulfonamide;
N-((1-(3,4-dichlorophenyl)cyclopropyl)methyl)-3-nitro-4-(trifluoromethoxy)benzenesulfonamide;
N-(1-(3,4-dichlorophenyl)-2-(piperazin-1-yl)ethyl)-4-(trifluoromethoxy)benzenesulfonamide;
N-(1-(3,4-dichlorophenyl)-2-(dimethylamino)ethyl)-3,5-difluorobenzenesulfonamide;
N-(1-(2-methoxy-5-methylphenyl)-3-methylbutan-2-yl)-4-(trifluoromethoxy)benzenesulfonamide;
N-(1-(3,4-dichlorophenyl)-2-(dimethylamino)ethyl)-4-(2-(trifluoromethyl)phenoxy)benzenesulfonamide;
N-(1-(3,4-dichlorophenyl)-2-(dimethylamino)ethyl)-4-(tetrahydro-2H-pyran-2-yl)benzenesulfonamide;
3-cyano-N-(1-(3,4-dichlorophenyl)-2-(dimethylamino)ethyl)-4-(trifluoromethoxy)benzenesulfonamide;
N-(1-(3,4-dichlorophenyl)-2-(4-propoxypiperidin-1-yl)ethyl)-4-(trifluoromethoxy)benzenesulfonamide;
N-(1-(3,4-dichlorophenyl)-2-(methylamino)ethyl)-4-(trifluoromethoxy)benzenesulfonamide;
N-((1-(3,4-dichlorophenyl)cyclopentyl)methyl)-4-(trifluoromethoxy)benzenesulfonamide;
N-((1-(4-chlorophenyl)cyclopentyl)methyl)-4-(trifluoromethoxy)benzenesulfonamide;
N-(2-(1-(3,4-dichlorophenyl)cyclopropyl)propan-2-yl)-4-(trifluoromethoxy)benzenesulfonamide;
N-(1-(3,4-dichlorophenyl)-3-(dimethylamino)propan-2-yl)-4-(trifluoromethoxy)benzenesulfonamide;
4-cyclobutoxy-N-(1-(3,4-dichlorophenyl)-2-(dimethylamino)ethyl)benzenesulfonamide;
3-(3,4-dichlorophenyl)-N,N-dimethyl-2-((4-(trifluoromethoxy)phenyl)sulfonamido)propenamide;
N-(1-(3,4-dichlorophenyl)-2-(dimethylamino)ethyl)-N-methyl-4-(trifluoromethoxy)benzenesulfonamide;
4-(cyclopentylamino)-N-(1-(3,4-dichlorophenyl)-2-(dimethylamino)ethyl)benzenesulfonamide;
4-(cyclobutylamino)-N-(1-(3,4-dichlorophenyl)-2-(dimethylamino)ethyl)benzenesulfonamide;
N-((3-(3,4-dichlorophenyl)pyrrolidin-3-yl)methyl)-4-(trifluoromethoxy)benzenesulfonamide;
2-(3,4-dichlorophenyl)-N,N-dimethyl-2-((4-(trifluoromethoxy)phenyl)sulfonamido)acetamide;
N-(1-(3,4-dichlorophenyl)-2-((4-methoxybenzyl)amino)ethyl)-4-(trifluoromethoxy)benzenesulfonamide;
N-(1-(4-chlorophenyl)-2-(piperazin-1-yl)ethyl)-4-(trifluoromethoxy)benzenesulfonamide;
N-(1-(4-chloro-3-fluorophenyl)-2-(piperazin-1-yl)ethyl)-4-(trifluoromethoxy)benzenesulfonamide;
N-(1-(4-chlorophenyl)-2-(piperazin-1-yl)ethyl)-6-(trifluoromethoxy)pyridine-3-sulfonamide;
N-((4-phenylpiperidin-4-yl)methyl)-4-(trifluoromethoxy)benzenesulfonamide;
N-(2-(2,5-diazabicyclo[2.2.1]heptan-2-yl)-1-(3,4-dichlorophenyl)ethyl)-4-(trifluoromethoxy)benzenesulfonamide;
4-(trifluoromethoxy)-N-((1-(3-(trifluoromethyl)phenyl)cyclopentyl)methyl)benzenesulfonamide;
N-((2-phenylpyrrolidin-2-yl)methyl)-4-(trifluoromethoxy)benzenesulfonamide;
N-(1-(4-fluorophenyl)-2-(piperazin-1-yl)ethyl)-4-(trifluoromethoxy)benzenesulfonamide;
N-(2-(4-fluorophenyl)-2-(piperazin-1-yl)ethyl)-4-(trifluoromethoxy)benzenesulfonamide;
N-(1-(3,4-dichlorophenyl)-2-(dimethylamino)ethyl)-3-nitro-4-(trifluoromethyl)benzenesulfonamide;
N-(1-(3,4-dichlorophenyl)-2-(dimethylamino)ethyl)-4-propoxybenzenesulfonamide;
4-(cyclohexylamino)-N-(1-(3,4-dichlorophenyl)-2-(dimethylamino)ethyl)benzenesulfonamide; and
N-(1-(3,4-dichlorophenyl)-2-(4-methylpiperazin-1-yl)ethyl)-4-(4-fluorophenoxy)benzenesulfonamide;
or a salt, solvate, isotopologue, or tautomer thereof.

28. The compound of claim 1, which is selected from the group consisting of:
(R)—N-(1-(3,4-dichlorophenyl)-2-(dimethylamino)ethyl)-3-nitro-4-(trifluoromethoxy)benzenesulfonamide;
(R)—N-(1-hydroxy-5-methylhexan-3-yl)-4-(trifluoromethoxy)benzenesulfonamide;
(R)—N-(1-(3,4-dichlorophenyl)-2-(dimethylamino)ethyl)-4-pentylbenzenesulfonamide;
(R)—N-(cyclohexyl(3,5-dichlorophenyl)methyl)-4-(trifluoromethoxy)benzenesulfonamide;
(R)—N-(1-(3,4-dichlorophenyl)-2-(dimethylamino)ethyl)-4-(4-(trifluoromethyl)phenoxy)benzenesulfonamide;
(R)—N-(1-(3,4-dichlorophenyl)-2-(dimethylamino)ethyl)-4-phenoxybenzenesulfonamide;
(R)—N-(1-(3,4-dichlorophenyl)-2-(dimethylamino)ethyl)-[1,1′-biphenyl]-4-sulfonamide;
(R)—N-(1-(4-chlorophenyl)-2-(dimethylamino)ethyl)-4-phenoxybenzenesulfonamide;
(R)—N-(2-(dimethylamino)-1-(3-(trifluoromethyl)phenyl)ethyl)-4-phenoxybenzenesulfonamide;
(R)-3-chloro-N-(1-(3,4-dichlorophenyl)-2-(dimethylamino)ethyl)-4-(trifluoromethoxy)benzenesulfonamide;
(R)-4-(cyclopentyloxy)-N-(1-(3,4-dichlorophenyl)-2-(dimethylamino)ethyl)benzenesulfonamide;
(R)—N-(1-(3,4-dichlorophenyl)-2-(dimethylamino)ethyl)-4-propylbenzenesulfonamide;
(R)—N-(1-(3,4-dichlorophenyl)-2-(4-methylpiperazin-1-yl)ethyl)-4-(trifluoromethoxy)benzenesulfonamide;
(R)—N-(1-(3,4-dichlorophenyl)-2-(dimethylamino)ethyl)-[1,1′-biphenyl]-3-sulfonamide;
(R)-3-chloro-N-(1-(4-chlorophenyl)-2-(dimethylamino)ethyl)-4-(trifluoromethoxy)benzenesulfonamide;
(R)-3-chloro-N-(2-(dimethylamino)-1-(3-(trifluoromethyl)phenyl)ethyl)-4-(trifluoromethoxy)benzenesulfonamide;
(R)—N-(1-(3-bromo-5-fluorophenyl)-2-cyclopropylethyl)-4-(trifluoromethoxy)benzenesulfonamide;
(R)—N-((1-(2,4-difluorophenyl)-2,2-dimethylcyclopropyl)methyl)-4-(trifluoromethoxy)benzenesulfonamide;
(R)—N-(1-(3,4-dichlorophenyl)-2-(dimethylamino)ethyl)-4-(trifluoromethoxy)benzenesulfonamide;
(R)—N-(2-(dimethylamino)-1-(3-(trifluoromethyl)phenyl)ethyl)-4-(4-(trifluoromethyl)phenoxy)benzenesulfonamide;
(R)—N-(2-(isopropyl(methyl)amino)benzyl)-4-(trifluoromethoxy)benzenesulfonamide;
(R)-4-(trifluoromethoxy)-N-(1-(4-(trifluoromethyl)phenyl)pent-4-en-1-yl)benzenesulfonamide;
N—((R)-((1R,2R)-2-methylcyclopropyl)(phenyl)methyl)-4-(trifluoromethoxy)benzenesulfonamide;
N—((R)-((1R,2S)-2-methylcyclopropyl)(phenyl)methyl)-4-(trifluoromethoxy)benzenesulfonamide;
(R)—N-(1-(3,4-dichlorophenyl)-2-(dimethylamino)ethyl)-3-(trifluoromethoxy)benzenesulfonamide;
(R)-2-amino-N-(1-(3,4-dichlorophenyl)-2-(dimethylamino)ethyl)-4-(trifluoromethoxy)benzenesulfonamide;
(R)—N-(1-(3,4-dichlorophenyl)-2-(dimethylamino)ethyl)naphthalene-1-sulfonamide;
(R)-2-chloro-N-(1-(3,4-dichlorophenyl)-2-(dimethylamino)ethyl)-4-(trifluoromethoxy)benzenesulfonamide;
(R)—N-(1-(3,4-dichlorophenyl)-2-(dimethylamino)ethyl)naphthalene-2-sulfonamide;
(R)-3,4-dichloro-N-(1-(3,4-dichlorophenyl)-2-(dimethylamino)ethyl)benzenesulfonamide;
N-(((2R,3R)-2-(4-methoxyphenyl)-1-methylpyrrolidin-3-yl)methyl)-4-(trifluoromethoxy)benzenesulfonamide;
N-(((2R,3 S)-2-(4-methoxyphenyl)-1-methylpyrrolidin-3-yl)methyl)-4-(trifluoromethoxy)benzenesulfonamide;
(R)—N-(1-hydroxy-3-(m-tolyl)propan-2-yl)-4-(trifluoromethoxy)benzenesulfonamide;
(R)—N-(1-(3,4-dichlorophenyl)-2-(dimethylamino)ethyl)-2-phenylethane-1-sulfonamide;
(R)—N-(1-(3,4-dichlorophenyl)-2-(pyrrolidin-1-yl)ethyl)-4-(trifluoromethoxy)benzenesulfonamide;
(R)—N-(1-(3,4-dichlorophenyl)-2-(dimethylamino)ethyl)-N′-methyl-4-(trifluoromethoxy)benzenesulfonimidamide;
(R)—N-(1-(3,4-dichlorophenyl)-2-(dimethylamino)ethyl)-2,2-difluorobenzo[d][1,3]dioxole-5-sulfonamide;
(R)-ethyl 3-((4-(trifluoromethoxy)phenyl)sulfonamido)-3-(3-(trifluoromethyl)phenyl)propanoate;
(R)—N-(1-(1-(4-bromophenyl)cyclopropyl)ethyl)-4-(trifluoromethoxy)benzenesulfonamide;
(R)-4-methyl-N-(thiazol-2-yl)-2-((4-(trifluoromethoxy)phenyl)sulfonamido)pentanamide;
(R)—N-(1-(3,4-dichlorophenyl)-2-(dimethylamino)ethyl)-4-isopropylbenzenesulfonamide;
(R)—N-(1-(3,4-dichlorophenyl)-2-(dimethylamino)ethyl)-4-(trifluoromethyl)benzenesulfonamide;
(R)—N-(4,4,4-trifluoro-1-(2,4,5-trifluorophenyl)butan-2-yl)-4-(trifluoromethoxy)benzenesulfonamide;
(R)—N-((6-benzyl-6-azaspiro[3.4]octan-8-yl)methyl)-4-(trifluoromethoxy)benzenesulfonamide;
(R)-4-chloro-N-(1-(3,4-dichlorophenyl)-2-(dimethylamino)ethyl)benzenesulfonamide;
(R)—N-(1-(3-cyanophenyl)-3-methylbutyl)-4-(trifluoromethoxy)benzenesulfonamide;
(R)—N-(cyclohexyl(3-(trifluoromethyl)phenyl)methyl)-4-(trifluoromethoxy)benzenesulfonamide;
(R,E)-N-(1-(3,4-dichlorophenyl)-2-(dimethylamino)ethyl)-2-phenylethene-1-sulfonamide;
(R)—N-(2-(2-hydroxypropoxy)benzyl)-4-(trifluoromethoxy)benzenesulfonamide;
(R)-3-amino-N-(1-(3,4-dichlorophenyl)-2-(dimethylamino)ethyl)-4-(trifluoromethoxy)benzenesulfonamide;
N-((1 S)-(4-fluorophenyl)(tetrahydrofuran-2-yl)methyl)-4-(trifluoromethoxy)benzenesulfonamide;
(R)—N-(1-(4-chlorophenyl)-2-(piperidin-1-yl)ethyl)-4-(trifluoromethoxy)benzenesulfonamide;
N—((R)-1-((R)-1-methylpiperidin-2-yl)propan-2-yl)-4-(trifluoromethoxy)benzenesulfonamide;
N—((R)-1-((S)-1-methylpiperidin-2-yl)propan-2-yl)-4-(trifluoromethoxy)benzenesulfonamide;
(R)—N-(1-(3,4-dichlorophenyl)-2-(dimethylamino)ethyl)-4-ethylbenzenesulfonamide;
(R)—N-(1-(2,5-difluorophenyl)propyl)-4-(trifluoromethoxy)benzenesulfonamide;
methyl (R)-3-(3,5-dichlorophenyl)-3-((4-(trifluoromethoxy)phenyl)sulfonamido)propanoate;
(R)—N-((3-chlorophenyl)(cyclopentyl)methyl)-4-(trifluoromethoxy)benzenesulfonamide;
(R)—N-(1-(benzo[d][1,3]dioxol-5-yl)butyl)-4-(trifluoromethoxy)benzenesulfonamide;
methyl (R)-3-(3-fluoro-4-hydroxyphenyl)-2-((4-(trifluoromethoxy)phenyl)sulfonamido)propanoate;
methyl (R)-3-(3-bromo-4-methoxyphenyl)-3-((4-(trifluoromethoxy)phenyl)sulfonamido)propanoate;
tert-butyl (R)-2-((4-(trifluoromethoxy)phenyl)sulfonamido)hexanoate;
(R)—N-(cyclopropyl(4-fluoro-3-(trifluoromethyl)phenyl)methyl)-4-(trifluoromethoxy)benzenesulfonamide;
(R)—N-(1-(3,4-dichlorophenyl)-2-hydroxyethyl)-4-(trifluoromethoxy)benzenesulfonamide;
(R)—N-(1-(3,4-dichlorophenyl)-2-(dimethylamino)ethyl)-3-methylbenzenesulfonamide;
methyl (R)-2-(2-bromophenyl)-2-((4-(trifluoromethoxy)phenyl)sulfonamido)acetate;
tert-butyl (R)-4,4-difluoro-2-(((4-(trifluoromethoxy)phenyl)sulfonamido)methyl)pyrrolidine-1-carboxylate;
(R)—N-(1-(3-chlorophenyl)-2-(dimethylamino)ethyl)-4-(trifluoromethoxy)benzenesulfonamide;
(R)—N-(2-morpholino-2-(4-(trifluoromethyl)phenyl)ethyl)-4-(trifluoromethoxy)benzenesulfonamide;
(R)—N-(2-(dimethylamino)-1-(p-tolyl)ethyl)-4-(trifluoromethoxy)benzenesulfonamide;
(R)—N-(1-(3,4-dichlorophenyl)-2-(dimethylamino)ethyl)-4-(difluoromethoxy)benzenesulfonamide;
(R)—N-(2-(furan-2-yl)-2-(4-methylpiperidin-1-yl)ethyl)-4-(trifluoromethoxy)benzenesulfonamide;
ethyl (R)-3-(2-chlorophenyl)-2-((4-(trifluoromethoxy)phenyl)sulfonamido)propanoate;
(R)—N-(2-(tert-butoxy)benzyl)-4-(trifluoromethoxy)benzenesulfonamide;
(R)-3-chloro-N-(1-(3,4-dichlorophenyl)-2-(dimethylamino)ethyl)benzenesulfonamide;
(R)—N-(2-(dimethylamino)-1-(3-(trifluoromethyl)phenyl)ethyl)-4-(trifluoromethoxy)benzenesulfonamide;
(R,R)—N-(1-(3,4-dichlorophenyl)-2-(dimethylamino)ethyl)-4-(trifluoromethoxy)benzenesulfonimidamide;
(R,S)—N-(1-(3,4-dichlorophenyl)-2-(dimethylamino)ethyl)-4-(trifluoromethoxy)benzenesulfonimidamide;
(R)—N-(1-(5-fluoro-2-(4-hydroxypiperidin-1-yl)phenyl)ethyl)-4-(trifluoromethoxy)benzenesulfonamide;
(R)—N-(2-(dimethylamino)-1-(m-tolyl)ethyl)-4-(trifluoromethoxy)benzenesulfonamide;
ethyl (R)-3-(3-bromophenyl)-3-((4-(trifluoromethoxy)phenyl)sulfonamido)propanoate;
(R)—N-(1-(3,4-dichlorophenyl)-2-(dimethylamino)ethyl)-2-methylbenzenesulfonamide;
(R)-4-chloro-N-(1-(3,4-dichlorophenyl)-2-(dimethylamino)ethyl)-3-fluorobenzenesulfonamide;
ethyl (R)-3-(3-chlorophenyl)-2-((4-(trifluoromethoxy)phenyl)sulfonamido)propanoate;
(R)—N-(1-(2-bromo-4-fluorophenyl)-2-cyclopropylethyl)-4-(trifluoromethoxy)benzenesulfonamide;
ethyl (R)-2-(3-bromo-4-methoxyphenyl)-2-((4-(trifluoromethoxy)phenyl)sulfonamido)acetate;
methyl (R)-3-(2-hydroxyphenyl)-2-((4-(trifluoromethoxy)phenyl)sulfonamido)propanoate;
(R)—N-(1-(3,4-dichlorophenyl)-3-methoxypropyl)-4-(trifluoromethoxy)benzenesulfonamide;
(R)—N-(1-(3,4-dichlorophenyl)-2-(dimethylamino)ethyl)-4-ethoxybenzenesulfonamide;
(R)—N-(1-(4-chlorophenyl)-2-(pyrrolidin-1-yl)ethyl)-4-(trifluoromethoxy)benzenesulfonamide;
(R)—N-(1-(3,4-dichlorophenyl)-2-(dimethylamino)ethyl)-4-methylbenzenesulfonamide;
(R)—N′-(1-(3,4-dichlorophenyl)-2-(dimethylamino)ethyl)-N-methyl-4-(trifluoromethoxy)benzenesulfonimidamide;
(R)—N-(1-(3,4-dichlorophenyl)-2-(dimethylamino)ethyl)-3-(trifluoromethyl)benzenesulfonamide;
(R)—N-cyclohexyl-4-(N-(1-(3,4-dichlorophenyl)-2-(dimethylamino)ethyl)sulfamoyl)benzamide;
(R)—N-(1-(3,4-dichlorophenyl)-2-morpholinoethyl)-4-(trifluoromethoxy)benzenesulfonamide;
(R)—N-cycloheptyl-N-methyl-2-((4-(trifluoromethoxy)phenyl)sulfonamido)pentanamide;
(R)—N-(4,4,4-trifluoro-3-phenylbutyl)-4-(trifluoromethoxy)benzenesulfonamide;
(R)—N-(1-(5-ethylfuran-2-yl)-2,2-dimethylpropyl)-4-(trifluoromethoxy)benzenesulfonamide;
(R)—N-(2-(4-ethylphenyl)-2-(pyrrolidin-1-yl)ethyl)-4-(trifluoromethoxy)benzenesulfonamide;
(R)—N-(1-(4-chlorophenyl)-2-(dimethylamino)ethyl)-4-(trifluoromethoxy)benzenesulfonamide;
methyl (R)-3-(3,4-dichlorophenyl)-2-((4-(trifluoromethoxy)phenyl)sulfonamido)propanoate;
(R)—N-(1-(3,4-dichlorophenyl)-2-(dimethylamino)ethyl)cyclohexanesulfonamide;
ethyl (R)-3-(2,5-dichlorothiophen-3-yl)-2-((4-(trifluoromethoxy)phenyl)sulfonamido)propanoate;
tert-butyl ((4-(trifluoromethoxy)phenyl)sulfonyl)-D-isoleucinate;
tert-butyl ((4-(trifluoromethoxy)phenyl)sulfonyl)-D-alloisoleucinate (R)-4-chloro-N-(1-(3,4-dichlorophenyl)-2-(dimethylamino)ethyl)-2-fluorobenzenesulfonamide;
(R)—N-(1-(3-bromophenyl)-4,4,4-trifluorobutan-2-yl)-4-(trifluoromethoxy)benzenesulfonamide;
(R)—N-(2-(dimethylamino)-1-(o-tolyl)ethyl)-4-(trifluoromethoxy)benzenesulfonamide;
methyl (R)-3-cyclohexyl-3-((4-(trifluoromethoxy)phenyl)sulfonamido)propanoate;
methyl (R)-4-phenyl-3-((4-(trifluoromethoxy)phenyl)sulfonamido)butanoate;
(R)—N-(1-(3,4-dichlorophenyl)-2-(dimethylamino)ethyl)-4-fluorobenzenesulfonamide;
(R)-3-(4-isopropylphenyl)-3-((4-(trifluoromethoxy)phenyl)sulfonamido)propanoic acid;
methyl (R)-2-phenyl-2-((4-(trifluoromethoxy)phenyl)sulfonamido)butanoate;
(R)—N-(1-(3,4-dichlorophenyl)-2-(dimethylamino)ethyl)-3-fluorobenzenesulfonamide;
ethyl (R)-4-methyl-2-((4-(trifluoromethoxy)phenyl)sulfonamido)hexanoate;
(R)—N-(3-(1-cyanopropoxy)benzyl)-4-(trifluoromethoxy)benzenesulfonamide;
ethyl (R)-3-(4-methoxyphenyl)-2-((4-(trifluoromethoxy)phenyl)sulfonamido)propanoate;
ethyl (R)-3-(4-isopropoxy-3-methoxyphenyl)-3-((4-(trifluoromethoxy)phenyl)sulfonamido)propanoate;
(R)—N-((4-chlorophenyl)(cyclopentyl)methyl)-4-(trifluoromethoxy)benzenesulfonamide;
ethyl (R)-3-(4-ethoxyphenyl)-3-((4-(trifluoromethoxy)phenyl)sulfonamido)propanoate;
(R)—N-(1-(3,4-dichlorophenyl)-2-(dimethylamino)ethyl)-3,5-difluorobenzenesulfonamide;
(R)—N-(1-(2-methoxy-5-methylphenyl)-3-methylbutan-2-yl)-4-(trifluoromethoxy)benzenesulfonamide;
N-(2-(((1R,2R)-2-methylcyclohexyl)oxy)ethyl)-4-(trifluoromethoxy)benzenesulfonamide;
N-(2-(((1R,2S)-2-methylcyclohexyl)oxy)ethyl)-4-(trifluoromethoxy)benzenesulfonamide;
N-(((1R,2R)-2-phenylcyclohexyl)methyl)-4-(trifluoromethoxy)benzenesulfonamide;
N-(((1R,2S)-2-phenylcyclohexyl)methyl)-4-(trifluoromethoxy)benzenesulfonamide;
N-(((1R,3R)-1-(dimethylamino)-3-methylcyclohexyl)methyl)-4-(trifluoromethoxy)benzenesulfonamide;
N-(((1R,3 S)-1-(dimethylamino)-3-methylcyclohexyl)methyl)-4-(trifluoromethoxy)benzenesulfonamide;
N-((1R,2R)-2-(methylsulfonyl)-1-(p-tolyl)propyl)-4-(trifluoromethoxy)benzenesulfonamide;
N-((1R,2S)-2-(methylsulfonyl)-1-(p-tolyl)propyl)-4-(trifluoromethoxy)benzenesulfonamide;
N-((1R,2R)-1-(2-chlorophenyl)-1-methoxypropan-2-yl)-4-(trifluoromethoxy)benzenesulfonamide;
N-((1R,2S)-1-(2-chlorophenyl)-1-methoxypropan-2-yl)-4-(trifluoromethoxy)benzenesulfonamide;
ethyl (R)-3-((R)-tetrahydrofuran-3-yl)-2-((4-(trifluoromethoxy)phenyl)sulfonamido)propanoate;
ethyl (R)-3-((S)-tetrahydrofuran-3-yl)-2-((4-(trifluoromethoxy)phenyl)sulfonamido)propanoate;
ethyl (R)-3-((R)-cyclohex-3-en-1-yl)-2-((4-(trifluoromethoxy)phenyl)sulfonamido)propanoate;
ethyl (R)-3-((S)-cyclohex-3-en-1-yl)-2-((4-(trifluoromethoxy)phenyl)sulfonamido)propanoate;
N—((R)-(4-fluorophenyl)((R)-tetrahydrofuran-2-yl)methyl)-4-(trifluoromethoxy)benzenesulfonamide;
N—((R)-(4-fluorophenyl)((S)-tetrahydrofuran-2-yl)methyl)-4-(trifluoromethoxy)benzenesulfonamide;
(R)-4-cyclopropoxy-N-(1-(3,4-dichlorophenyl)-2-(dimethylamino)ethyl)benzenesulfonamide;
(R)—N-(1-(3-chloro-4-fluorophenyl)-2-(dimethylamino)ethyl)-4-(trifluoromethoxy)benzenesulfonamide;
(R)—N-(1-(4-chloro-3-fluorophenyl)-2-(dimethylamino)ethyl)-4-(trifluoromethoxy)benzenesulfonamide;
(R)-4-(N-(1-(3,4-dichlorophenyl)-2-(dimethylamino)ethyl)sulfamoyl)-N-phenylbenzamide;
(R)—N-(1-(3,4-dichlorophenyl)-2-(dimethylamino)ethyl)-4-(phenylamino)benzenesulfonamide;
(R)—N-(1-(4-chlorophenyl)-2-(dimethylamino)ethyl)-4-(4-(trifluoromethyl)phenoxy)benzenesulfonamide;
(R)—N-(2-(dimethylamino)-1-(3-(trifluoromethyl)phenyl)ethyl)-3-nitro-4-(trifluoromethoxy)benzenesulfonamide;
(R)—N-(1-(4-chlorophenyl)-2-(dimethylamino)ethyl)-3-nitro-4-(trifluoromethoxy)benzenesulfonamide;
(R)-4-(cyclopropylmethoxy)-N-(1-(3,4-dichlorophenyl)-2-(dimethylamino)ethyl)benzenesulfonamide;
(R)—N-(1-(3,4-dichlorophenyl)-2-(dimethylamino)ethyl)-4-isopropoxybenzenesulfonamide;
(R)-4-(cyclohexyloxy)-N-(1-(3,4-dichlorophenyl)-2-(dimethylamino)ethyl)benzenesulfonamide;
(R)—N-(1-(3,4-dichlorophenyl)-2-(dimethylamino)ethyl)-4-(pyridin-2-yloxy)benzenesulfonamide;
(R)-4-butyl-N-(1-(3,4-dichlorophenyl)-2-(dimethylamino)ethyl)benzenesulfonamide;
(R)—N-(1-(3,4-dichlorophenyl)-2-(dimethylamino)ethyl)-4-(3,6-dihydro-2H-pyran-4-yl)benzenesulfonamide;
(R)—N-(2-(dimethylamino)-1-(4-(trifluoromethyl)phenyl)ethyl)-4-(trifluoromethoxy)benzenesulfonamide;
(R)—N-(1-(3,4-dichlorophenyl)-2-(dimethylamino)ethyl)-4-(3-fluorophenoxy)benzenesulfonamide;
(R)—N-(1-(3,4-dichlorophenyl)-2-(dimethylamino)ethyl)-4-(4-fluorophenoxy)benzenesulfonamide;
(R)-methyl 5-(N-(1-(3,4-dichlorophenyl)-2-(dimethylamino)ethyl)sulfamoyl)-2-(trifluoromethoxy)benzoate;
(R)—N-(1-(3,4-dichlorophenyl)-2-(4-methylpiperazin-1-yl)ethyl)-4-(4-(trifluoromethyl)phenoxy)benzenesulfonamide;
(R)—N-(1-(4-chlorophenyl)-2-(4-methylpiperazin-1-yl)ethyl)-4-phenoxybenzenesulfonamide;
(R)—N-(1-(4-chlorophenyl)-2-(4-methylpiperazin-1-yl)ethyl)-4-(4-(trifluoromethyl)phenoxy)benzenesulfonamide;
(R)—N-(1-(3,4-dichlorophenyl)-2-(4-methylpiperazin-1-yl)ethyl)-4-phenoxybenzenesulfonamide;
(R)—N-(2-(dimethylamino)-1-(3-(trifluoromethyl)phenyl)ethyl)-4-(4-(trifluoromethyl)phenoxy)benzenesulfonamide;
(R)—N-(1-(3,4-dichlorophenyl)-2-(dimethylamino)ethyl)-6-(trifluoromethoxy)pyridine-3-sulfonamide;
(R)—N-(1-(3,4-dichlorophenyl)-2-(dimethylamino)ethyl)-3-nitro-4-(trifluoromethoxy)benzenesulfonamide;
(R)—N-(1-(3,4-dichlorophenyl)-2-(dimethylamino)ethyl)-4-(pyridin-2-yl)benzenesulfonamide;
(R)—N-(1-(3,4-dichlorophenyl)-2-(dimethylamino)ethyl)-4-nitro-3-(trifluoromethoxy)benzenesulfonamide;
(R)—N-(2-amino-1-(3,4-dichlorophenyl)ethyl)-4-(trifluoromethoxy)benzenesulfonamide hydrochloride;
(R)-tert-butyl (2-(3,4-dichlorophenyl)-2-((4-(trifluoromethoxy)phenyl)sulfonamido)ethyl)carbamate;
(R)—N-((2,2-dimethyl-1-phenylcyclopropyl)methyl)-4-(trifluoromethoxy)benzenesulfonamide;
(R)—N-(1-(3,4-dichlorophenyl)-2-(dimethylamino)ethyl)-2-nitro-3-(trifluoromethoxy)benzenesulfonamide;
4-(trifluoromethoxy)-N-((1R,3R)-3,4,4-trimethylcyclohexyl)benzenesulfonamide;
4-(trifluoromethoxy)-N-((1R,3 S)-3,4,4-trimethylcyclohexyl)benzenesulfonamide;
(R)—N-(1-(3,4-dichlorophenyl)-2-(dimethylamino)ethyl)-4-(2-(trifluoromethyl)phenoxy)benzenesulfonamide;
(R)—N-(1-(3,4-dichlorophenyl)-2-(dimethylamino)ethyl)-4-(tetrahydro-2H-pyran-2-yl)benzenesulfonamide;
(R)-3-cyano-N-(1-(3,4-dichlorophenyl)-2-(dimethylamino)ethyl)-4-(trifluoromethoxy)benzenesulfonamide;
(R)—N-(1-(3,4-dichlorophenyl)-2-(4-propoxypiperidin-1-yl)ethyl)-4-(trifluoromethoxy)benzenesulfonamide;
(R)—N-(1-(3,4-dichlorophenyl)-2-(methylamino)ethyl)-4-(trifluoromethoxy)benzenesulfonamide;
(R)—N-(1-(3,4-dichlorophenyl)-3-(dimethylamino)propan-2-yl)-4-(trifluoromethoxy)benzenesulfonamide;
(R)-4-cyclobutoxy-N-(1-(3,4-dichlorophenyl)-2-(dimethylamino)ethyl)benzenesulfonamide;
(R)-3-(3,4-dichlorophenyl)-N,N-dimethyl-2-((4-(trifluoromethoxy)phenyl)sulfonamido)propenamide;
(R)—N-(1-(3,4-dichlorophenyl)-2-(dimethylamino)ethyl)-N-methyl-4-(trifluoromethoxy)benzenesulfonamide;
(R)-4-(cyclopentylamino)-N-(1-(3,4-dichlorophenyl)-2-(dimethylamino)ethyl)benzenesulfonamide;
(R)-4-(cyclobutylamino)-N-(1-(3,4-dichlorophenyl)-2-(dimethylamino)ethyl)benzenesulfonamide;
(R)—N-((3-(3,4-dichlorophenyl)pyrrolidin-3-yl)methyl)-4-(trifluoromethoxy)benzenesulfonamide;
(R)-2-(3,4-dichlorophenyl)-N,N-dimethyl-2-((4-(trifluoromethoxy)phenyl)sulfonamido)acetamide;
(R)—N-(1-(3,4-dichlorophenyl)-2-((4-methoxybenzyl)amino)ethyl)-4-(trifluoromethoxy)benzenesulfonamide;
(R)—N-(1-(4-chlorophenyl)-2-(piperazin-1-yl)ethyl)-4-(trifluoromethoxy)benzenesulfonamide;
(R)—N-(1-(4-chloro-3-fluorophenyl)-2-(piperazin-1-yl)ethyl)-4-(trifluoromethoxy)benzenesulfonamide;
(R)—N-(1-(4-chlorophenyl)-2-(piperazin-1-yl)ethyl)-6-(trifluoromethoxy)pyridine-3-sulfonamide;
N—((R)-2-((1R,4R)-2,5-diazabicyclo[2.2.1]heptan-2-yl)-1-(3,4-dichlorophenyl)ethyl)-4-(trifluoromethoxy)benzenesulfonamide;
N—((R)-2-((1S,4S)-2,5-diazabicyclo[2.2.1]heptan-2-yl)-1-(3,4-dichlorophenyl)ethyl)-4-(trifluoromethoxy)benzenesulfonamide;
(R)—N-((2-phenylpyrrolidin-2-yl)methyl)-4-(trifluoromethoxy)benzenesulfonamide;
(R)—N-(1-(4-fluorophenyl)-2-(piperazin-1-yl)ethyl)-4-(trifluoromethoxy)benzenesulfonamide;
(R)—N-(2-(4-fluorophenyl)-2-(piperazin-1-yl)ethyl)-4-(trifluoromethoxy)benzenesulfonamide;
(R)—N-(1-(3,4-dichlorophenyl)-2-(dimethylamino)ethyl)-3-nitro-4-(trifluoromethyl)benzenesulfonamide;
(R)—N-(1-(3,4-dichlorophenyl)-2-(dimethylamino)ethyl)-4-propoxybenzenesulfonamide;
(R)-4-(cyclohexylamino)-N-(1-(3,4-dichlorophenyl)-2-(dimethylamino)ethyl)benzenesulfonamide; and
(R)—N-(1-(3,4-dichlorophenyl)-2-(4-methylpiperazin-1-yl)ethyl)-4-(4-fluorophenoxy)benzenesulfonamide;
(S)—N-(1-(3,4-dichlorophenyl)-2-(dimethylamino)ethyl)-3-nitro-4-(trifluoromethoxy)benzenesulfonamide;
(S)—N-(1-hydroxy-5-methylhexan-3-yl)-4-(trifluoromethoxy)benzenesulfonamide;
(S)—N-(1-(3,4-dichlorophenyl)-2-(dimethylamino)ethyl)-4-pentylbenzenesulfonamide;
(S)—N-(cyclohexyl(3,5-dichlorophenyl)methyl)-4-(trifluoromethoxy)benzenesulfonamide;
(S)—N-(1-(3,4-dichlorophenyl)-2-(dimethylamino)ethyl)-4-(4-(trifluoromethyl)phenoxy)benzenesulfonamide;
(S)—N-(1-(3,4-dichlorophenyl)-2-(dimethylamino)ethyl)-4-phenoxybenzenesulfonamide;
(S)—N-(1-(3,4-dichlorophenyl)-2-(dimethylamino)ethyl)-[1,1′-biphenyl]-4-sulfonamide;
(S)—N-(1-(4-chlorophenyl)-2-(dimethylamino)ethyl)-4-phenoxybenzenesulfonamide;
(S)—N-(2-(dimethylamino)-1-(3-(trifluoromethyl)phenyl)ethyl)-4-phenoxybenzenesulfonamide;
(S)-3-chloro-N-(1-(3,4-dichlorophenyl)-2-(dimethylamino)ethyl)-4-(trifluoromethoxy)benzenesulfonamide;
(S)-4-(cyclopentyloxy)-N-(1-(3,4-dichlorophenyl)-2-(dimethylamino)ethyl)benzenesulfonamide;
(S)—N-(1-(3,4-dichlorophenyl)-2-(dimethylamino)ethyl)-4-propylbenzenesulfonamide;
(S)—N-(1-(3,4-dichlorophenyl)-2-(4-methylpiperazin-1-yl)ethyl)-4-(trifluoromethoxy)benzenesulfonamide;
(S)—N-(1-(3,4-dichlorophenyl)-2-(dimethylamino)ethyl)-[1,1′-biphenyl]-3-sulfonamide;
(S)-3-chloro-N-(1-(4-chlorophenyl)-2-(dimethylamino)ethyl)-4-(trifluoromethoxy)benzenesulfonamide;
(S)-3-chloro-N-(2-(dimethylamino)-1-(3-(trifluoromethyl)phenyl)ethyl)-4-(trifluoromethoxy)benzenesulfonamide;
(S)—N-(1-(3-bromo-5-fluorophenyl)-2-cyclopropylethyl)-4-(trifluoromethoxy)benzenesulfonamide;
(S)—N-((1-(2,4-difluorophenyl)-2,2-dimethylcyclopropyl)methyl)-4-(trifluoromethoxy)benzenesulfonamide;
(S)—N-(1-(3,4-dichlorophenyl)-2-(dimethylamino)ethyl)-4-(trifluoromethoxy)benzenesulfonamide;
(S)—N-(2-(dimethylamino)-1-(3-(trifluoromethyl)phenyl)ethyl)-4-(4-(trifluoromethyl)phenoxy)benzenesulfonamide;
(S)—N-(2-(isopropyl(methyl)amino)benzyl)-4-(trifluoromethoxy)benzenesulfonamide;
(S)-4-(trifluoromethoxy)-N-(1-(4-(trifluoromethyl)phenyl)pent-4-en-1-yl)benzenesulfonamide;
N—((S)-((1S,2S)-2-methylcyclopropyl)(phenyl)methyl)-4-(trifluoromethoxy)benzenesulfonamide;
N—((S)-((1S,2R)-2-methylcyclopropyl)(phenyl)methyl)-4-(trifluoromethoxy)benzenesulfonamide;
(S)—N-(1-(3,4-dichlorophenyl)-2-(dimethylamino)ethyl)-3-(trifluoromethoxy)benzenesulfonamide;
(S)-2-amino-N-(1-(3,4-dichlorophenyl)-2-(dimethylamino)ethyl)-4-(trifluoromethoxy)benzenesulfonamide;
(S)—N-(1-(3,4-dichlorophenyl)-2-(dimethylamino)ethyl)naphthalene-1-sulfonamide;
(S)-2-chloro-N-(1-(3,4-dichlorophenyl)-2-(dimethylamino)ethyl)-4-(trifluoromethoxy)benzenesulfonamide;
(S)—N-(1-(3,4-dichlorophenyl)-2-(dimethylamino)ethyl)naphthalene-2-sulfonamide;
(S)-3,4-dichloro-N-(1-(3,4-dichlorophenyl)-2-(dimethylamino)ethyl)benzenesulfonamide;
N-(((2S,3S)-2-(4-methoxyphenyl)-1-methylpyrrolidin-3-yl)methyl)-4-(trifluoromethoxy)benzenesulfonamide;
N-(((2S,3R)-2-(4-methoxyphenyl)-1-methylpyrrolidin-3-yl)methyl)-4-(trifluoromethoxy)benzenesulfonamide;
(S)—N-(1-hydroxy-3-(m-tolyl)propan-2-yl)-4-(trifluoromethoxy)benzenesulfonamide;
(S)—N-(1-(3,4-dichlorophenyl)-2-(dimethylamino)ethyl)-2-phenylethane-1-sulfonamide;
(S)—N-(1-(3,4-dichlorophenyl)-2-(pyrrolidin-1-yl)ethyl)-4-(trifluoromethoxy)benzenesulfonamide;
(S)—N-(1-(3,4-dichlorophenyl)-2-(dimethylamino)ethyl)-N′-methyl-4-(trifluoromethoxy)benzenesulfonimidamide;
(S)—N-(1-(3,4-dichlorophenyl)-2-(dimethylamino)ethyl)-2,2-difluorobenzo[d][1,3]dioxole-5-sulfonamide;
(S)-ethyl 3-((4-(trifluoromethoxy)phenyl)sulfonamido)-3-(3-(trifluoromethyl)phenyl)propanoate;
(S)—N-(1-(1-(4-bromophenyl)cyclopropyl)ethyl)-4-(trifluoromethoxy)benzenesulfonamide;
(S)-4-methyl-N-(thiazol-2-yl)-2-((4-(trifluoromethoxy)phenyl)sulfonamido)pentanamide;
(S)—N-(1-(3,4-dichlorophenyl)-2-(dimethylamino)ethyl)-4-isopropylbenzenesulfonamide;
(S)—N-(1-(3,4-dichlorophenyl)-2-(dimethylaminolethyl)-4-(trifluoromethyl)benzenesulfonamide;
(S)—N-(4,4,4-trifluoro-1-(2,4,5-trifluorophenyl)butan-2-yl)-4-(trifluoromethoxy)benzenesulfonamide;
(S)—N-((6-benzyl-6-azaspiro[3.4]octan-8-yl)methyl)-4-(trifluoromethoxy)benzenesulfonamide;
(S)-4-chloro-N-(1-(3,4-dichlorophenyl)-2-(dimethylamino)ethyl)benzenesulfonamide;
(S)—N-(1-(3-cyanophenyl)-3-methylbutyl)-4-(trifluoromethoxy)benzenesulfonamide;
(S)—N-(cyclohexyl(3-(trifluoromethyl)phenyl)methyl)-4-(trifluoromethoxy)benzenesulfonamide;
(S,E)-N-(1-(3,4-dichlorophenyl)-2-(dimethylamino)ethyl)-2-phenylethene-1-sulfonamide;
(S)—N-(2-(2-hydroxypropoxy)benzyl)-4-(trifluoromethoxy)benzenesulfonamide;
(S)-3-amino-N-(1-(3,4-dichlorophenyl)-2-(dimethylamino)ethyl)-4-(trifluoromethoxy)benzenesulfonamide;
N-((1 S)-(4-fluorophenyl)(tetrahydrofuran-2-yl)methyl)-4-(trifluoromethoxy)benzenesulfonamide;
(S)—N-(1-(4-chlorophenyl)-2-(piperidin-1-yl)ethyl)-4-(trifluoromethoxy)benzenesulfonamide;
N—((S)-1-((S)-1-methylpiperidin-2-yl)propan-2-yl)-4-(trifluoromethoxy)benzenesulfonamide;
N—((S)-1-((R)-1-methylpiperidin-2-yl)propan-2-yl)-4-(trifluoromethoxy)benzenesulfonamide;
(S)—N-(1-(3,4-dichlorophenyl)-2-(dimethylamino)ethyl)-4-ethylbenzenesulfonamide;
(S)—N-(1-(2,5-difluorophenyl)propyl)-4-(trifluoromethoxy)benzenesulfonamide;
methyl (S)-3-(3,5-dichlorophenyl)-3-((4-(trifluoromethoxy)phenyl)sulfonamido)propanoate;
(S)—N-((3-chlorophenyl)(cyclopentyl)methyl)-4-(trifluoromethoxy)benzenesulfonamide;
(S)—N-(1-(benzo[d][1,3]dioxol-5-yl)butyl)-4-(trifluoromethoxy)benzenesulfonamide;
methyl (S)-3-(3-fluoro-4-hydroxyphenyl)-2-((4-(trifluoromethoxy)phenyl)sulfonamido)propanoate;
methyl (S)-3-(3-bromo-4-methoxyphenyl)-3-((4-(trifluoromethoxy)phenyl)sulfonamido)propanoate;
tert-butyl (S)-2-((4-(trifluoromethoxy)phenyl)sulfonamido)hexanoate;
(S)—N-(cyclopropyl(4-fluoro-3-(trifluoromethyl)phenyl)methyl)-4-(trifluoromethoxy)benzenesulfonamide;
(S)—N-(1-(3,4-dichlorophenyl)-2-hydroxyethyl)-4-(trifluoromethoxy)benzenesulfonamide;
(S)—N-(1-(3,4-dichlorophenyl)-2-(dimethylamino)ethyl)-3-methylbenzenesulfonamide;
methyl (S)-2-(2-bromophenyl)-2-((4-(trifluoromethoxy)phenyl)sulfonamido)acetate;
tert-butyl (S)-4,4-difluoro-2-(((4-(trifluoromethoxy)phenyl)sulfonamido)methyl)pyrrolidine-1-carboxylate;
(S)—N-(1-(3-chlorophenyl)-2-(dimethylamino)ethyl)-4-(trifluoromethoxy)benzenesulfonamide;
(S)—N-(2-morpholino-2-(4-(trifluoromethyl)phenyl)ethyl)-4-(trifluoromethoxy)benzenesulfonamide;
(S)—N-(2-(dimethylamino)-1-(p-tolyl)ethyl)-4-(trifluoromethoxy)benzenesulfonamide;
(S)—N-(1-(3,4-dichlorophenyl)-2-(dimethylamino)ethyl)-4-(difluoromethoxy)benzenesulfonamide;
(S)—N-(2-(furan-2-yl)-2-(4-methylpiperidin-1-yl)ethyl)-4-(trifluoromethoxy)benzenesulfonamide;
ethyl (S)-3-(2-chlorophenyl)-2-((4-(trifluoromethoxy)phenyl)sulfonamido)propanoate;
(S)—N-(2-(tert-butoxy)benzyl)-4-(trifluoromethoxy)benzenesulfonamide;
(S)-3-chloro-N-(1-(3,4-dichlorophenyl)-2-(dimethylamino)ethyl)benzenesulfonamide;
(S)—N-(2-(dimethylamino)-1-(3-(trifluoromethyl)phenyl)ethyl)-4-(trifluoromethoxy)benzenesulfonamide;
(S,S)—N-(1-(3,4-dichlorophenyl)-2-(dimethylamino)ethyl)-4-(trifluoromethoxy)benzenesulfonimidamide;
(S,R)—N-(1-(3,4-dichlorophenyl)-2-(dimethylamino)ethyl)-4-(trifluoromethoxy)benzenesulfonimidamide;
(S)—N-(1-(5-fluoro-2-(4-hydroxypiperidin-1-yl)phenyl)ethyl)-4-(trifluoromethoxy)benzenesulfonamide;
(S)—N-(2-(dimethylamino)-1-(m-tolyl)ethyl)-4-(trifluoromethoxy)benzenesulfonamide;
ethyl (S)-3-(3-bromophenyl)-3-((4-(trifluoromethoxy)phenyl)sulfonamido)propanoate;
(S)—N-(1-(3,4-dichlorophenyl)-2-(dimethylaminolethyl)-2-methylbenzenesulfonamide;
(S)-4-chloro-N-(1-(3,4-dichlorophenyl)-2-(dimethylamino)ethyl)-3-fluorobenzenesulfonamide;
ethyl (S)-3-(3-chlorophenyl)-2-((4-(trifluoromethoxy)phenyl)sulfonamido)propanoate;
(S)—N-(1-(2-bromo-4-fluorophenyl)-2-cyclopropylethyl)-4-(trifluoromethoxy)benzenesulfonamide;
ethyl (S)-2-(3-bromo-4-methoxyphenyl)-2-((4-(trifluoromethoxy)phenyl)sulfonamido)acetate;
methyl (S)-3-(2-hydroxyphenyl)-2-((4-(trifluoromethoxy)phenyl)sulfonamido)propanoate;
(S)—N-(1-(3,4-dichlorophenyl)-3-methoxypropyl)-4-(trifluoromethoxy)benzenesulfonamide;
(S)—N-(1-(3,4-dichlorophenyl)-2-(dimethylamino)ethyl)-4-ethoxybenzenesulfonamide;
(S)—N-(1-(4-chlorophenyl)-2-(pyrrolidin-1-yl)ethyl)-4-(trifluoromethoxy)benzenesulfonamide;
(S)—N-(1-(3,4-dichlorophenyl)-2-(dimethylaminolethyl)-4-methylbenzenesulfonamide;
(S)—N′-(1-(3,4-dichlorophenyl)-2-(dimethylamino)ethyl)-N-methyl-4-(trifluoromethoxy)benzenesulfonimidamide;
(S)—N-(1-(3,4-dichlorophenyl)-2-(dimethylaminolethyl)-3-(trifluoromethyl)benzenesulfonamide;
(S)—N-cyclohexyl-4-(N-(1-(3,4-dichlorophenyl)-2-(dimethylamino)ethyl)sulfamoyl)benzamide;
(S)—N-(1-(3,4-dichlorophenyl)-2-morpholinoethyl)-4-(trifluoromethoxy)benzenesulfonamide;
(S)—N-cycloheptyl-N-methyl-2-((4-(trifluoromethoxy)phenyl)sulfonamido)pentanamide;
(S)—N-(4,4,4-trifluoro-3-phenylbutyl)-4-(trifluoromethoxy)benzenesulfonamide;
(S)—N-(1-(5-ethylfuran-2-yl)-2,2-dimethylpropyl)-4-(trifluoromethoxy)benzenesulfonamide;
(S)—N-(2-(4-ethylphenyl)-2-(pyrrolidin-1-yl)ethyl)-4-(trifluoromethoxy)benzenesulfonamide;
(S)—N-(1-(4-chlorophenyl)-2-(dimethylamino)ethyl)-4-(trifluoromethoxy)benzenesulfonamide;
methyl (S)-3-(3,4-dichlorophenyl)-2-((4-(trifluoromethoxy)phenyl)sulfonamido)propanoate;
(S)—N-(1-(3,4-dichlorophenyl)-2-(dimethylamino)ethyl)cyclohexanesulfonamide;
ethyl (S)-3-(2,5-dichlorothiophen-3-yl)-2-((4-(trifluoromethoxy)phenyl)sulfonamido)propanoate tert-butyl ((4-(trifluoromethoxy)phenyl)sulfonyl)-L-isoleucinate;
tert-butyl ((4-(trifluoromethoxy)phenyl)sulfonyl)-L-alloisoleucinate (S)-4-chloro-N-(1-(3,4-dichlorophenyl)-2-(dimethylamino)ethyl)-2-fluorobenzenesulfonamide;
(S)—N-(1-(3-bromophenyl)-4,4,4-trifluorobutan-2-yl)-4-(trifluoromethoxy)benzenesulfonamide;
(S)—N-(2-(dimethylamino)-1-(o-tolyl)ethyl)-4-(trifluoromethoxy)benzenesulfonamide;
methyl (S)-3-cyclohexyl-3-((4-(trifluoromethoxy)phenyl)sulfonamido)propanoate;
methyl (S)-4-phenyl-3-((4-(trifluoromethoxy)phenyl)sulfonamido)butanoate;
(S)—N-(1-(3,4-dichlorophenyl)-2-(dimethylamino)ethyl)-4-fluorobenzenesulfonamide;
(S)-3-(4-isopropylphenyl)-3-((4-(trifluoromethoxy)phenyl)sulfonamido)propanoic acid;
methyl (S)-2-phenyl-2-((4-(trifluoromethoxy)phenyl)sulfonamido)butanoate;
(S)—N-(1-(3,4-dichlorophenyl)-2-(dimethylamino)ethyl)-3-fluorobenzenesulfonamide;
ethyl (S)-4-methyl-2-((4-(trifluoromethoxy)phenyl)sulfonamido)hexanoate;
(S)—N-(3-(1-cyanopropoxy)benzyl)-4-(trifluoromethoxy)benzenesulfonamide;
ethyl (S)-3-(4-methoxyphenyl)-2-((4-(trifluoromethoxy)phenyl)sulfonamido)propanoate;
ethyl (S)-3-(4-isopropoxy-3-methoxyphenyl)-3-((4-(trifluoromethoxy)phenyl)sulfonamido)propanoate;
(S)—N-((4-chlorophenyl)(cyclopentyl)methyl)-4-(trifluoromethoxy)benzenesulfonamide;
ethyl (S)-3-(4-ethoxyphenyl)-3-((4-(trifluoromethoxy)phenyl)sulfonamido)propanoate;
(S)—N-(1-(3,4-dichlorophenyl)-2-(dimethylamino)ethyl)-3,5-difluorobenzenesulfonamide;
(S)—N-(1-(2-methoxy-5-methylphenyl)-3-methylbutan-2-yl)-4-(trifluoromethoxy)benzenesulfonamide;
N-(2-(((1S,2S)-2-methylcyclohexyl)oxy)ethyl)-4-(trifluoromethoxy)benzenesulfonamide;
N-(2-(((1S,2R)-2-methylcyclohexyl)oxy)ethyl)-4-(trifluoromethoxy)benzenesulfonamide;
N-(((1S,2S)-2-phenylcyclohexyl)methyl)-4-(trifluoromethoxy)benzenesulfonamide;
N-(((1S,2R)-2-phenylcyclohexyl)methyl)-4-(trifluoromethoxy)benzenesulfonamide;
N-(((1S,3S)-1-(dimethylamino)-3-methylcyclohexyl)methyl)-4-(trifluoromethoxy)benzenesulfonamide;
N-(((1S,3R)-1-(dimethylamino)-3-methylcyclohexyl)methyl)-4-(trifluoromethoxy)benzenesulfonamide;
N-((1S,2S)-2-(methylsulfonyl)-1-(p-tolyl)propyl)-4-(trifluoromethoxy)benzenesulfonamide;
N-((1S,2R)-2-(methylsulfonyl)-1-(p-tolyl)propyl)-4-(trifluoromethoxy)benzenesulfonamide;
N-((1S,2S)-1-(2-chlorophenyl)-1-methoxypropan-2-yl)-4-(trifluoromethoxy)benzenesulfonamide;
N-((1S,2R)-1-(2-chlorophenyl)-1-methoxypropan-2-yl)-4-(trifluoromethoxy)benzenesulfonamide;
ethyl (S)-3-((S)-tetrahydrofuran-3-yl)-2-((4-(trifluoromethoxy)phenyl)sulfonamido)propanoate;
ethyl (S)-3-((R)-tetrahydrofuran-3-yl)-2-((4-(trifluoromethoxy)phenyl)sulfonamido)propanoate;
ethyl (S)-3-((S)-cyclohex-3-en-1-yl)-2-((4-(trifluoromethoxy)phenyl)sulfonamido)propanoate;
ethyl (S)-3-((R)-cyclohex-3-en-1-yl)-2-((4-(trifluoromethoxy)phenyl)sulfonamido)propanoate;
N—((S)-(4-fluorophenyl)((S)-tetrahydrofuran-2-yl)methyl)-4-(trifluoromethoxy)benzenesulfonamide;
N—((S)-(4-fluorophenyl)((R)-tetrahydrofuran-2-yl)methyl)-4-(trifluoromethoxy)benzenesulfonamide;
(S)-4-cyclopropoxy-N-(1-(3,4-dichlorophenyl)-2-(dimethylamino)ethyl)benzenesulfonamide;
(S)—N-(1-(3-chloro-4-fluorophenyl)-2-(dimethylamino)ethyl)-4-(trifluoromethoxy)benzenesulfonamide;
(S)—N-(1-(4-chloro-3-fluorophenyl)-2-(dimethylamino)ethyl)-4-(trifluoromethoxy)benzenesulfonamide;
(S)-4-(N-(1-(3,4-dichlorophenyl)-2-(dimethylamino)ethyl)sulfamoyl)-N-phenylbenzamide;
(S)—N-(1-(3,4-dichlorophenyl)-2-(dimethylaminolethyl)-4-(phenylamino)benzenesulfonamide;
(S)—N-(1-(4-chlorophenyl)-2-(dimethylamino)ethyl)-4-(4-(trifluoromethyl)phenoxy)benzenesulfonamide;
(S)—N-(2-(dimethylamino)-1-(3-(trifluoromethyl)phenyl)ethyl)-3-nitro-4-(trifluoromethoxy)benzenesulfonamide;
(S)—N-(1-(4-chlorophenyl)-2-(dimethylamino)ethyl)-3-nitro-4-(trifluoromethoxy)benzenesulfonamide;
(S)-4-(cyclopropylmethoxy)-N-(1-(3,4-dichlorophenyl)-2-(dimethylamino)ethyl)benzenesulfonamide;
(S)—N-(1-(3,4-dichlorophenyl)-2-(dimethylamino)ethyl)-4-isopropoxybenzenesulfonamide;
(S)-4-(cyclohexyloxy)-N-(1-(3,4-dichlorophenyl)-2-(dimethylamino)ethyl)benzenesulfonamide;
(S)—N-(1-(3,4-dichlorophenyl)-2-(dimethylamino)ethyl)-4-(pyridin-2-yloxy)benzenesulfonamide;
(S)-4-butyl-N-(1-(3,4-dichlorophenyl)-2-(dimethylamino)ethyl)benzenesulfonamide;
(S)—N-(1-(3,4-dichlorophenyl)-2-(dimethylamino)ethyl)-4-(3,6-dihydro-2H-pyran-4-yl)benzenesulfonamide;
(S)—N-(2-(dimethylamino)-1-(4-(trifluoromethyl)phenyl)ethyl)-4-(trifluoromethoxy)benzenesulfonamide;
(S)—N-(1-(3,4-dichlorophenyl)-2-(dimethylamino)ethyl)-4-(3-fluorophenoxy)benzenesulfonamide;
(S)—N-(1-(3,4-dichlorophenyl)-2-(dimethylamino)ethyl)-4-(4-fluorophenoxy)benzenesulfonamide;
(S)-methyl 5-(N-(1-(3,4-dichlorophenyl)-2-(dimethylamino)ethyl)sulfamoyl)-2-(trifluoromethoxy)benzoate;
(S)—N-(1-(3,4-dichlorophenyl)-2-(4-methylpiperazin-1-yl)ethyl)-4-(4-(trifluoromethyl)phenoxy)benzenesulfonamide;
(S)—N-(1-(4-chlorophenyl)-2-(4-methylpiperazin-1-yl)ethyl)-4-phenoxybenzenesulfonamide;
(S)—N-(1-(4-chlorophenyl)-2-(4-methylpiperazin-1-yl)ethyl)-4-(4-(trifluoromethyl)phenoxy)benzenesulfonamide;
(S)—N-(1-(3,4-dichlorophenyl)-2-(4-methylpiperazin-1-yl)ethyl)-4-phenoxybenzenesulfonamide;
(S)—N-(2-(dimethylamino)-1-(3-(trifluoromethyl)phenyl)ethyl)-4-(4-(trifluoromethyl)phenoxy)benzenesulfonamide;
(S)—N-(1-(3,4-dichlorophenyl)-2-(dimethylamino)ethyl)-6-(trifluoromethoxy)pyridine-3-sulfonamide;
(S)—N-(1-(3,4-dichlorophenyl)-2-(dimethylamino)ethyl)-3-nitro-4-(trifluoromethoxy)benzenesulfonamide;
(S)—N-(1-(3,4-dichlorophenyl)-2-(dimethylamino)ethyl)-4-(pyridin-2-yl)benzenesulfonamide;
(S)—N-(1-(3,4-dichlorophenyl)-2-(dimethylamino)ethyl)-4-nitro-3-(trifluoromethoxy)benzenesulfonamide;
(S)—N-(2-amino-1-(3,4-dichlorophenyl)ethyl)-4-(trifluoromethoxy)benzenesulfonamide hydrochloride;
(S)-tert-butyl (2-(3,4-dichlorophenyl)-2-((4-(trifluoromethoxy)phenyl)sulfonamido)ethyl)carbamate;
(S)—N-((2,2-dimethyl-1-phenylcyclopropyl)methyl)-4-(trifluoromethoxy)benzenesulfonamide;
(S)—N-(1-(3,4-dichlorophenyl)-2-(dimethylamino)ethyl)-2-nitro-3-(trifluoromethoxy)benzenesulfonamide;
4-(trifluoromethoxy)-N-((1S,3S)-3,4,4-trimethylcyclohexyl)benzenesulfonamide;
4-(trifluoromethoxy)-N-((1S,3R)-3,4,4-trimethylcyclohexyl)benzenesulfonamide;
(S)—N-(1-(3,4-dichlorophenyl)-2-(dimethylamino)ethyl)-4-(2-(trifluoromethyl)phenoxy)benzenesulfonamide;
(S)—N-(1-(3,4-dichlorophenyl)-2-(dimethylamino)ethyl)-4-(tetrahydro-2H-pyran-2-yl)benzenesulfonamide;
(S)-3-cyano-N-(1-(3,4-dichlorophenyl)-2-(dimethylamino)ethyl)-4-(trifluoromethoxy)benzenesulfonamide;
(S)—N-(1-(3,4-dichlorophenyl)-2-(4-propoxypiperidin-1-yl)ethyl)-4-(trifluoromethoxy)benzenesulfonamide;
(S)—N-(1-(3,4-dichlorophenyl)-2-(methylamino)ethyl)-4-(trifluoromethoxy)benzenesulfonamide;
(S)—N-(1-(3,4-dichlorophenyl)-3-(dimethylamino)propan-2-yl)-4-(trifluoromethoxy)benzenesulfonamide;
(S)-4-cyclobutoxy-N-(1-(3,4-dichlorophenyl)-2-(dimethylamino)ethyl)benzenesulfonamide;
(S)-3-(3,4-dichlorophenyl)-N,N-dimethyl-2-((4-(trifluoromethoxy)phenyl)sulfonamido)propenamide;
(S)—N-(1-(3,4-dichlorophenyl)-2-(dimethylamino)ethyl)-N-methyl-4-(trifluoromethoxy)benzenesulfonamide;
(S)-4-(cyclopentylamino)-N-(1-(3,4-dichlorophenyl)-2-(dimethylamino)ethyl)benzenesulfonamide;
(S)-4-(cyclobutylamino)-N-(1-(3,4-dichlorophenyl)-2-(dimethylamino)ethyl)benzenesulfonamide;
(S)—N-((3-(3,4-dichlorophenyl)pyrrolidin-3-yl)methyl)-4-(trifluoromethoxy)benzenesulfonamide;
(S)-2-(3,4-dichlorophenyl)-N,N-dimethyl-2-((4-(trifluoromethoxy)phenyl)sulfonamido)acetamide;
(S)—N-(1-(3,4-dichlorophenyl)-2-((4-methoxybenzyl)amino)ethyl)-4-(trifluoromethoxy)benzenesulfonamide;
(S)—N-(1-(4-chlorophenyl)-2-(piperazin-1-yl)ethyl)-4-(trifluoromethoxy)benzenesulfonamide;
(S)—N-(1-(4-chloro-3-fluorophenyl)-2-(piperazin-1-yl)ethyl)-4-(trifluoromethoxy)benzenesulfonamide;
(S)—N-(1-(4-chlorophenyl)-2-(piperazin-1-yl)ethyl)-6-(trifluoromethoxy)pyridine-3-sulfonamide;
N—((S)-2-((1R,4R)-2,5-diazabicyclo[2.2.1]heptan-2-yl)-1-(3,4-dichlorophenyl)ethyl)-4-(trifluoromethoxy)benzenesulfonamide;
N—((S)-2-((1S,4S)-2,5-diazabicyclo[2.2.1]heptan-2-yl)-1-(3,4-dichlorophenyl)ethyl)-4-(trifluoromethoxy)benzenesulfonamide;
(S)—N-((2-phenylpyrrolidin-2-yl)methyl)-4-(trifluoromethoxy)benzenesulfonamide;
(S)—N-(1-(4-fluorophenyl)-2-(piperazin-1-yl)ethyl)-4-(trifluoromethoxy)benzenesulfonamide;
(S)—N-(2-(4-fluorophenyl)-2-(piperazin-1-yl)ethyl)-4-(trifluoromethoxy)benzenesulfonamide;
(S)—N-(1-(3,4-dichlorophenyl)-2-(dimethylamino)ethyl)-3-nitro-4-(trifluoromethyl)benzenesulfonamide;
(S)—N-(1-(3,4-dichlorophenyl)-2-(dimethylamino)ethyl)-4-propoxybenzenesulfonamide;
(S)-4-(cyclohexylamino)-N-(1-(3,4-dichlorophenyl)-2-(dimethylamino)ethyl)benzenesulfonamide; and
(S)—N-(1-(3,4-dichlorophenyl)-2-(4-methylpiperazin-1-yl)ethyl)-4-(4-fluorophenoxy)benzenesulfonamide;
or a salt, solvate, isotopologue, or tautomer thereof.

29. (canceled)

30. A compound selected from the group consisting of:
N-(3,3,3-trifluoro-1-phenylpropyl)-4-(trifluoromethoxy)benzenesulfonamide;
(R)—N-(3,3,3-trifluoro-1-phenylpropyl)-4-(trifluoromethoxy)benzenesulfonamide;
(S)—N-(3,3,3-trifluoro-1-phenylpropyl)-4-(trifluoromethoxy)benzenesulfonamide;
N-(3-hydroxy-1-phenylpropyl)-4-(trifluoromethoxy)benzenesulfonamide;
(R)—N-(3-hydroxy-1-phenylpropyl)-4-(trifluoromethoxy)benzenesulfonamide;
(S)—N-(3-hydroxy-1-phenylpropyl)-4-(trifluoromethoxy)benzenesulfonamide;
N-(2-cyclopropyl-1-phenylethyl)-4-(trifluoromethoxy)benzenesulfonamide;
(R)—N-(2-cyclopropyl-1-phenylethyl)-4-(trifluoromethoxy)benzenesulfonamide;
(S)—N-(2-cyclopropyl-1-phenylethyl)-4-(trifluoromethoxy)benzenesulfonamide;
methyl 2-methyl-3-phenyl-3-((4-(trifluoromethoxy)phenyl)sulfonamido)propanoate;
methyl (2R,3R)-2-methyl-3-phenyl-3-((4-(trifluoromethoxy)phenyl)sulfonamido)propanoate;
methyl (2R,3S)-2-methyl-3-phenyl-3-((4-(trifluoromethoxy)phenyl)sulfonamido)propanoate;
methyl (2S,3S)-2-methyl-3-phenyl-3-((4-(trifluoromethoxy)phenyl)sulfonamido)propanoate;
methyl (2S,3R)-2-methyl-3-phenyl-3-((4-(trifluoromethoxy)phenyl)sulfonamido)propanoate;
ethyl 3-(p-tolyl)-3-((4-(trifluoromethoxy)phenyl)sulfonamido)propanoate;
ethyl (R)-3-(p-tolyl)-3-((4-(trifluoromethoxy)phenyl)sulfonamido)propanoate;
ethyl (S)-3-(p-tolyl)-3-((4-(trifluoromethoxy)phenyl)sulfonamido)propanoate;
N-(4,4,4-trifluoro-3-hydroxy-3-(trifluoromethyl)butyl)-4-(trifluoromethoxy)benzenesulfonamide;
N-((1-(2-methoxyethyl)cyclohexyl)methyl)-4-(trifluoromethoxy)benzenesulfonamide;
N-(2-(diisobutylamino)ethyl)-4-(trifluoromethoxy)benzenesulfonamide;
N-(3-(benzyl(butyl)amino)propyl)-4-(trifluoromethoxy)benzenesulfonamide;
N-(1-(2,6-dichlorophenyl)propan-2-yl)-4-(trifluoromethoxy)benzenesulfonamide;
(R)—N-(1-(2,6-dichlorophenyl)propan-2-yl)-4-(trifluoromethoxy)benzenesulfonamide;
(S)—N-(1-(2,6-dichlorophenyl)propan-2-yl)-4-(trifluoromethoxy)benzenesulfonamide;
tert-butyl 2,2-dimethyl-6-(((4-(trifluoromethoxy)phenyl)sulfonamido)methyl)morpholine-4-carboxylate;
(R)-tert-butyl 2,2-dimethyl-6-(((4-(trifluoromethoxy)phenyl)sulfonamido)methyl)morpholine-4-carboxylate;
(S)-tert-butyl 2,2-dimethyl-6-(((4-(trifluoromethoxy)phenyl)sulfonamido)methyl)morpholine-4-carboxylate;
N-(3-((furan-2-ylmethyl)((tetrahydrofuran-2-yl)methyl)amino)propyl)-4-(trifluoromethoxy)benzenesulfonamide;
(R)—N-(3-((furan-2-ylmethyl)((tetrahydrofuran-2-yl)methyl)amino)propyl)-4-(trifluoromethoxy)benzenesulfonamide;
(S)—N-(3-((furan-2-ylmethyl)((tetrahydrofuran-2-yl)methyl)amino)propyl)-4-(trifluoromethoxy)benzenesulfonamide;
N-((1-(2-(benzyloxy)ethyl)cyclopropyl)methyl)-4-(trifluoromethoxy)benzenesulfonamide;
N-(3-(2-(benzyloxy)ethoxy)propyl)-4-(trifluoromethoxy)benzenesulfonamide;
N-(1-(1-methylpiperidin-2-yl)propan-2-yl)-4-(trifluoromethoxy)benzenesulfonamide;
N-(2-((2-methylcyclohexyl)oxy)ethyl)-4-(trifluoromethoxy)benzenesulfonamide;
N-((1-((dimethylamino)methyl)cyclopropyl)methyl)-4-(trifluoromethoxy)benzenesulfonamide;
N-((1-(dimethylamino)-3-methylcyclohexyl)methyl)-4-(trifluoromethoxy)benzenesulfonamide;
N-(2-(2,2-dioxidobenzo[c][1,2,5]thiadiazol-1(3H)-yl)ethyl)-4-(trifluoromethoxy)benzenesulfonamide;
N-((3-methylcyclohexyl)(o-tolyl)methyl)-4-(trifluoromethoxy)benzenesulfonamide;
(R)—N-((3-methylcyclohexyl)(o-tolyl)methyl)-4-(trifluoromethoxy)benzenesulfonamide;
(S)—N-((3-methylcyclohexyl)(o-tolyl)methyl)-4-(trifluoromethoxy)benzenesulfonamide;
N-((2-methylcyclopropyl)(phenyl)methyl)-4-(trifluoromethoxy)benzenesulfonamide;
(R)—N-((2-methylcyclopropyl)(phenyl)methyl)-4-(trifluoromethoxy)benzenesulfonamide;
(S)—N-((2-methylcyclopropyl)(phenyl)methyl)-4-(trifluoromethoxy)benzenesulfonamide;
N-(2-(methylsulfonyl)-1-(p-tolyl)propyl)-4-(trifluoromethoxy)benzenesulfonamide;
tert-butyl (1-phenyl-2-((4-(trifluoromethoxy)phenyl)sulfonamido)propyl)carbamate;
tert-butyl ((1R,2R)-1-phenyl-2-((4-(trifluoromethoxy)phenyl)sulfonamido)propyl)carbamate;
tert-butyl ((1R,2S)-1-phenyl-2-((4-(trifluoromethoxy)phenyl)sulfonamido)propyl)carbamate;
tert-butyl ((1S,2S)-1-phenyl-2-((4-(trifluoromethoxy)phenyl)sulfonamido)propyl)carbamate;
tert-butyl ((1S,2R)-1-phenyl-2-((4-(trifluoromethoxy)phenyl)sulfonamido)propyl)carbamate;
tert-butyl (2-phenyl-2-((4-(trifluoromethoxy)phenyl)sulfonamido)ethyl)carbamate;
tert-butyl (R)-(2-phenyl-2-((4-(trifluoromethoxy)phenyl)sulfonamido)ethyl)carbamate;
tert-butyl (S)-(2-phenyl-2-((4-(trifluoromethoxy)phenyl)sulfonamido)ethyl)carbamate;
N-(2-amino-1-phenylethyl)-4-(trifluoromethoxy)benzenesulfonamide;
(R)—N-(2-amino-1-phenylethyl)-4-(trifluoromethoxy)benzenesulfonamide;
(S)—N-(2-amino-1-phenylethyl)-4-(trifluoromethoxy)benzenesulfonamide;
N-(1-phenyl-2-(4-propoxypiperidin-1-yl)ethyl)-4-(trifluoromethoxy)benzenesulfonamide;
(R)—N-(1-phenyl-2-(4-propoxypiperidin-1-yl)ethyl)-4-(trifluoromethoxy)benzenesulfonamide;
(S)—N-(1-phenyl-2-(4-propoxypiperidin-1-yl)ethyl)-4-(trifluoromethoxy)benzenesulfonamide;
1-methyl-N-(2-phenyl-2-((4-(trifluoromethoxy)phenyl)sulfonamido)ethyl)-1H-pyrrole-3-sulfonamide;
(R)-1-methyl-N-(2-phenyl-2-((4-(trifluoromethoxy)phenyl)sulfonamido)ethyl)-1H-pyrrole-3-sulfonamide;
(S)-1-methyl-N-(2-phenyl-2-((4-(trifluoromethoxy)phenyl)sulfonamido)ethyl)-1H-pyrrole-3-sulfonamide;
N-(1-(dimethylamino)propan-2-yl)-3-nitro-4-(trifluoromethoxy)benzenesulfonamide;
(R)—N-(1-(dimethylamino)propan-2-yl)-3-nitro-4-(trifluoromethoxy)benzenesulfonamide; and
(S)—N-(1-(dimethylamino)propan-2-yl)-3-nitro-4-(trifluoromethoxy)benzenesulfonamide;
or a salt, solvate, isotopologue, or tautomer thereof.

31. A pharmaceutical composition comprising at least one compound of claim 1 and at least one pharmaceutically acceptable carrier.

32. A method of treating, preventing, or ameliorating a PP2A-related disease in a subject, the method comprising administering to a subject in need thereof a therapeutically effective amount of the compound of claim 1 or a pharmaceutical composition thereof further comprising at least one pharmaceutically acceptable carrier.

33. The method of claim 32, wherein the PP2A-related disease is selected from the group consisting of cancer, diabetes, autoimmune disease, solid organ transplant rejection, graft vs host disease, chronic obstructive pulmonary disease (COPD), non-alcoholic fatty liver disease, abdominal aortic aneurysm, chronic liver disease, heart failure, neurodegenerative disease, and cardiac hypertrophy.

34. The method of claim 32, wherein the subject is a mammal, optionally wherein the mammal is a human.

35. (canceled)
